(19) United States
(12) Patent Application Publication
(10) Pub. No.: US 2022/0380319 A1
(43) Pub. Date: Dec. 1, 2022

(54) SUBSTITUTED BENZIMIDAZOLE CARBOXAMIDES AND THEIR USE IN THE TREATMENT OF MEDICAL DISORDERS

(71) Applicant: Bial - R&D Investments, S.A., Coronado (S.Romão e S.Mamede) (PT)

(72) Inventors: Renato T. Skerlj, West Newton, MA (US); Elyse Marie Josee Bourque, L'etang-Du-Nord (CA); Soumya Ray, Quincy, MA (US); Peter T. Lansbury, Brookline, MA (US)

(21) Appl. No.: 17/760,864

(22) PCT Filed: Sep. 17, 2020
(86) PCT No.: PCT/US2020/051251

§ 371 Date: Mar. 16, 2022

Related U.S. Application Data
(60) Provisional application No. 62/901,375, filed on Sep. 17, 2019.

(51) Int. Cl. C07D 235/04; C07D 235/26; C07D 401/10; C07D 403/10; C07D 471/04; C07D 401/12; C07D 417/10; C07D 405/12; C07D 487/04; C07D 471/08; C07D 491/08; C07D 491/107; C07D 403/12; C07D 417/12; C07D 498/04

(52) U.S. Cl. CPC C07D 235/04; C07D 235/26; C07D 401/10; C07D 403/10; C07D 471/04; C07D 401/12; C07D 417/10; C07D 405/12; C07D 487/04; C07D 471/08; C07D 491/08; C07D 491/107; C07D 403/12; C07D 417/12; C07D 498/04

(57) ABSTRACT

The invention provides substituted benzimidazole carboxamides and related compounds, compositions containing such compounds, medical kits, and methods for using such compounds and compositions to treat a medical disorder, e.g., cancer, lysosomal storage disorder, neurodegenerative disorder, inflammatory disorder, in a patient.

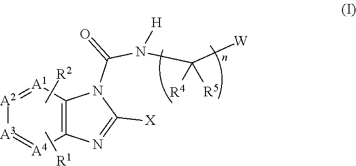

### CROSS-REFERENCE TO RELATED APPLICATIONS

This application claims the benefit of U.S. Provisional Patent Application No. 62/901,375 filed on Sep. 17, 2019, the entire contents of which are incorporated by reference herein.

### FIELD OF THE INVENTION

The invention provides substituted benzimidazole carboxamides and related compounds, compositions containing such compounds, medical kits, and methods for using such compounds and compositions to treat medical disorders in a patient.

### BACKGROUND

Sphingolipids, in addition to serving roles in cell membrane structure and dynamics, also serve important signaling functions, for example, in the control of cell growth, cell differentiation, and cell death, and so are important for cell homeostasis and development. Zeidan et al. (2010) CURR. MOL. MED. 10, 454, Proksch et al. (2011) J. LIPIDS Article ID 971618. Ceramide, a key member of this lipid class, has attracted attention in view of its impact on the replication and differentiation of neoplastic cells. Furuya et al. (2011) CANCER METASTASIS REV. 30, 567. For example, lower levels of ceramide have been discovered in several types of human tumors relative to normal tissue, where the level of ceramide appears to correlate inversely with the degree of malignant progression. Realini et al. (2013) J. MOL. BIOL. 56, 3518.

Acid ceramidase (AC, also known as N-acylsphingosine amidohydrolase-1, or ASAH-1) is a cysteine amidase that catalyzes the hydrolysis of ceramide into sphingosine and fatty acid. Acid ceramidase is believed to be involved in the regulation of ceramide levels in cells and modulates the ability of this lipid messenger to influence the survival, growth and death of certain tumor cells. Doan et al. ONCOTARGET 8(68), 112662-74, 2017. Furthermore, acid ceramidase enzymes are abnormally expressed in various types of human cancer (e.g., prostate, head and neck, and colon) and serum AC levels are elevated in patients with melanoma relative to control subjects. Realini et al. (2015) J. BIOL. CHEM. 291 (5), 2422-34.

In addition, acid ceramidase enzymes have been implicated in a number of other disorders, including, inflammation (for example, rheumatoid arthritis and psoriasis), pain, inflammatory pain, and various pulmonary disorders. See, International Application Publication No. WO2015/173169. Furthermore, acid ceramidase enzymes have been identified as a target for the treatment of certain lysosomal storage disorders (for example, Gaucher's, Fabry's, Krabbe, Tay Sachs), and neurodegenerative disorders (for example, Alzheimers, Parkinson's, Huntington's, and amytrophic lateral sclerosis). See, International Application Publication Nos. WO2016/210116 and WO2016/210120.

Despite the efforts to develop acid ceramidase inhibitors for use in the treatment of various disorders there is still a need for new acid ceramidase inhibitors.

### SUMMARY

The invention provides substituted benzimidazole carboxamides and related compounds, compositions containing such compounds, medical kits, and methods for using such compounds and compositions to treat medical disorders, for example, cancer (such as melanoma), a lysosomal storage disorder (such as Krabbe disease, Fabry disease, Tay-Sachs disease, Pompe disease, Hunter's syndrome, Niemann Pick disease Types A and B, Gaucher disease), a neurodegenerative disease (such as Alzheimer's disease, Parkinson's disease, Huntington's disease, amyotrophic lateral sclerosis, and Lewy body disease), an inflammatory disorder, and pain. Various aspects and embodiments of the invention are described in further detail below.

In one aspect, provided herein is a compound embraced by formula (I):

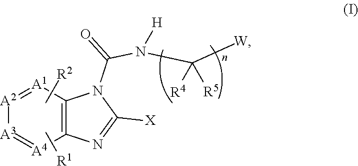

or a pharmaceutically acceptable salt thereof, wherein: R¹ is selected from the group consisting of hydrogen, phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl); R² is selected from the group consisting of hydrogen, halogen, C₁₋₆alkyl, C₁₋₆haloalkyl, —OR^{c}, and cyano; R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene; n is integer selected from 0 to 6; X is selected from the group consisting of hydrogen, deuterium, —OR^{b}, —S(C₁₋₆alkyl), C₁₋₆ alkyl, and phenyl; R^{a} is independently, for each occurrence, hydrogen or C₁₋₆alkyl; R^{a} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂; R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl; and A¹, A², A³, and A⁴ are CH, or one or two of A¹, A², A³, and A⁴ are N and the others are CH; and wherein any aforementioned phenyl, C₃₋₇cycloalkyl, 3-10 membered heterocyclyl, —(C₂₋₆alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted; wherein, the compound is not a compound of

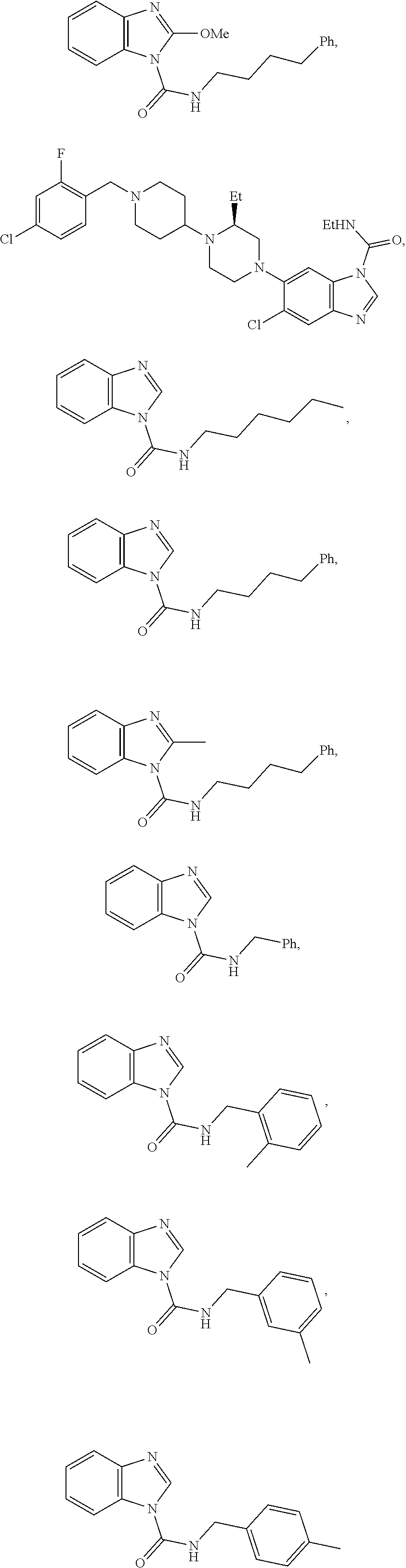

or a pharmaceutically acceptable salt thereof.

In another aspect, provided herein is a compound of formula (I-a):

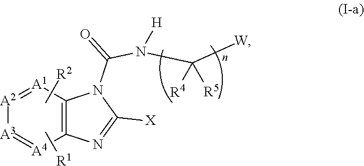

or a pharmaceutically acceptable salt thereof, wherein the variables are as defined herein.

In another aspect, provided herein is a compound of formula (I-b):

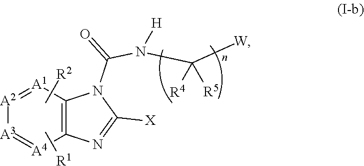

or a pharmaceutically acceptable salt thereof, wherein the variables are as defined herein.

In another aspect, provided herein is a compound of formula (I-c):

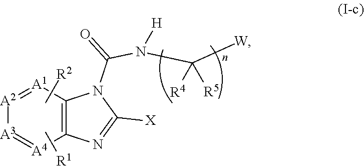

or a pharmaceutically acceptable salt thereof, wherein the variables are as defined herein.

In some embodiments, the compound is a compound of formula (I-d):

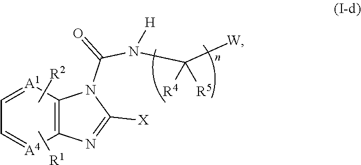

or a pharmaceutically acceptable salt thereof, wherein the variables are as defined herein.

In some embodiments, the compound is a compound of formula (I-e):

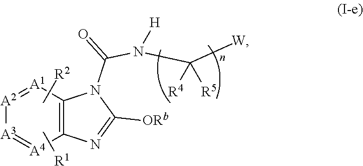

or a pharmaceutically acceptable salt thereof, wherein the variables are as defined herein.

In some embodiments, the compound is a compound of formula (I-f):

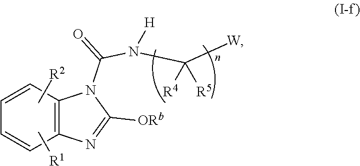

or a pharmaceutically acceptable salt thereof, wherein the variables are as defined herein.

In certain embodiments, the compound is a compound of formula (I-g):

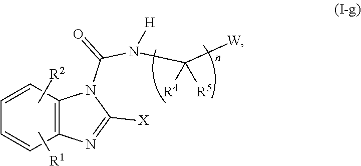

or a pharmaceutically acceptable salt thereof, wherein the variables are as defined herein.

In some embodiments, the compound is a compound of formula (I-h):

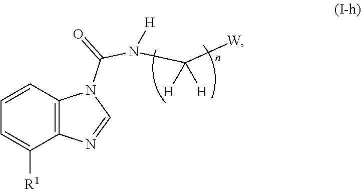

or a pharmaceutically acceptable salt thereof, wherein the variables are as defined herein.

In another aspect, provided herein is a compound of formula (II):

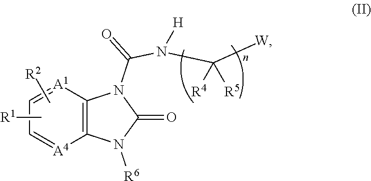

or a pharmaceutically acceptable salt thereof, wherein: A¹ and A⁴ are independently selected from CH and N; R¹ is selected from the group consisting of hydrogen, phenyl, C₃₋₇cycloalkyl, 3-7 membered heterocyclyl, 5-6 membered heteroaryl, C₁₋₆alkylene-N(R^{a})₂, (3-7 membered heterocyclene)-(3-7 membered heterocyclyl), (5-6 membered heteroarylene)-(3-7 membered heterocyclyl) and —C(O)-(3-7 membered heterocyclyl); R² is selected from the group consisting of hydrogen, halogen, C₁₋₆alkyl, C₁₋₆haloalkyl, —OR^{c}, and cyano; R⁴ and R⁵ are independently, for each occurrence, selected from the group consisting of hydrogen, C₁₋₆alkyl, C₁₋₆haloalkyl, and halogen; R⁶ is hydrogen or C₁₋₂ alkyl; n is integer between 0 to 6; R is independently hydrogen or C₁₋₆alkyl; R^{b} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; and W is selected from the group consisting of methyl, halogen, phenyl, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, and —(C₂₋₆alkynylene)-C₃₋₇cycloalkyl wherein any aforementioned phenyl, 3-7 membered heterocyclyl, or 5-6 membered heteroaryl is optionally substituted, wherein, the compound is not a compound of

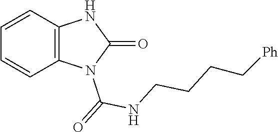 [SMILES: O=C(NCCCCc1ccccc1)n1c(=O)[nH]c2ccccc21]

or a pharmaceutically acceptable salt thereof.

In another aspect, provided herein is a compound of formula (II-a):

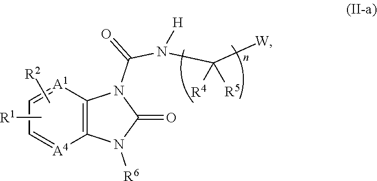

or a pharmaceutically acceptable salt thereof, wherein:

A¹ and A⁴ are independently selected from CH and N; R¹ is selected from the group consisting of hydrogen, phenyl, C₃₋₇cycloalkyl, 3-7 membered heterocyclyl, 5-6 membered heteroaryl, C₁₋₆alkylene-N(R^{a})₂, (3-7 membered heterocyclene)-(3-7 membered heterocyclyl), (5-6 membered heteroarylene)-(3-7 membered heterocyclyl) and —C(O)-(3-7 membered heterocyclyl); R² is selected from the group consisting of hydrogen, halogen, C₁₋₆alkyl, C₁₋₆haloalkyl, —OR^{c}, and cyano; R⁴ and R⁵ are independently, for each occurrence, selected from the group consisting of hydrogen, C₁₋₆alkyl, C₁₋₆haloalkyl, and halogen; R⁶ is hydrogen or C₁₋₂ alkyl; R^{a} is independently hydrogen or C₁₋₆alkyl; R^{b} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; and W is selected from the group consisting of methyl, halogen, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, and —(C₂₋₆alkynylene)-C₃₋₇cycloalkyl, and n is integer selected from 0, 1, 2, 3, 4, 5, or 6, or W is phenyl, and n is integer selected from 0, 1, 2, 3, 5, or 6, wherein any aforementioned phenyl, 3-7 membered heterocyclyl, or 5-6 membered heteroaryl is optionally substituted.

In another aspect, provided herein is a pharmaceutical composition comprising a compound disclosed herein (e.g., a compound of Formula (I) or (II) (e.g., a compound of Formula (I-a), (I-b), (I-c), (I-d), (I-e), (I-f), (I-g), (I-h), or (II-a)) and a pharmaceutically acceptable carrier.

In another aspect, the invention provides a method of treating a subject with cancer and in need thereof, the method comprising administering to the subject a therapeutically effective amount of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition disclosed herein.

In another aspect, the invention provides a method of treating a subject with a lysosomal storage disorder and in need thereof, the method comprising administering to the subject a therapeutically effective amount of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition disclosed herein.

In another aspect, the invention provides a method of treating a subject with a neurodegenerative disorder and in need thereof, the method comprising administering to the subject a therapeutically effective amount of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition disclosed herein.

In another aspect, the invention provides a method of treating a subject with an inflammatory disorder and in need thereof, the method comprising administering to the subject a therapeutically effective amount of a compound (e.g., a compound of Formula (I) or (II) (e.g., a compound of Formula (I-a), (I-b), (I-c), (I-d), (I-e), (I-f), (I-g), (I-h), or (I-a)) or a pharmaceutical composition disclosed herein.

In another aspect, the invention provides a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for use in a method of treating a subject with cancer and in need thereof, the method comprising administering to the subject a therapeutically effective amount of the compound or the pharmaceutical composition.

In another aspect, the invention provides a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for use in a method of treating a subject with a lysosomal storage disorder and in need thereof, the method comprising administering to the subject a therapeutically effective amount of the compound or the pharmaceutical composition.

In another aspect, the invention provides a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for use in a method of treating a subject with a neurodegenerative disorder and in need thereof, the method comprising administering to the subject a therapeutically effective amount of the compound or the pharmaceutical composition.

In another aspect, the invention provides a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for use in a method of treating a subject with an inflammatory disorder and in need thereof, the method comprising administering to the subject a therapeutically effective amount of the compound or the pharmaceutical composition.

In another aspect, the invention provides use of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for the manufacture of a medicament for treating a subject with cancer and in need thereof, the method comprising administering to the subject a therapeutically effective amount of the compound or the pharmaceutical composition.

In another aspect, the invention provides use of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for the manufacture of a medicament for treating a subject with a lysosomal storage disorder and in need thereof, the method comprising administering to the subject a therapeutically effective amount of the compound or the pharmaceutical composition.

In another aspect, the invention provides use of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for the manufacture of a medicament for treating a subject with a neurodegenerative disorder and in need thereof, the method comprising administering to the subject a therapeutically effective amount of the compound or the pharmaceutical composition.

In another aspect, the invention provides use of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for the manufacture of a medicament for treating a subject with an inflammatory disorder and in need thereof, the method comprising administering to the subject a therapeutically effective amount of the compound or the pharmaceutical composition.

### DETAILED DESCRIPTION

The invention provides substituted benzimidazole carboxamides and related compounds, compositions containing such compounds, medical kits, and methods for using such compounds and compositions to treat medical disorders in a patient. The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, and the like.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂alkynyl, C₂-C₁₀alkynyl, and C₂-C₆alkynyl, respectively. Exemplary alkynyl groups include ethynyl, prop-1-yn-1-yl, and but-1-yn-1-yl.

The term “cycloalkyl” refers to a monovalent saturated cyclic, bicyclic, bridged cyclic (e.g., adamantyl), or spirocyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkene” refers to a diradical of an cycloalkyl group. An exemplary cycloalkylene group is

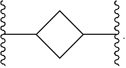

The term “cycloalkenyl” as used herein refers to a monovalent unsaturated cyclic, bicyclic, or bridged cyclic (e.g., adamantyl) hydrocarbon group of 3-12, 3-8, 4-8, or 4-6 carbons containing one carbon-carbon double bond, referred to herein, e.g., as “C₄₋₈cycloalkenyl,” derived from a cycloalkane. Exemplary cycloalkenyl groups include, but are not limited to, cyclohexenes, cyclopentenes, and cyclobutenes. Unless specified otherwise, cycloalkenyl groups are optionally substituted at one or more ring positions with, for example, alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkenyl group is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “aralkyl” refers to an alkyl group substituted with an aryl group.

The term “bicyclic carbocyclyl that is partially unsaturated” refers to a bicyclic carbocyclic group containing at least one double bond between ring atoms and at least one ring in the bicyclic carbocyclic group is not aromatic. Representative examples of a bicyclic carbocyclyl that is partially unsaturated include, for example:

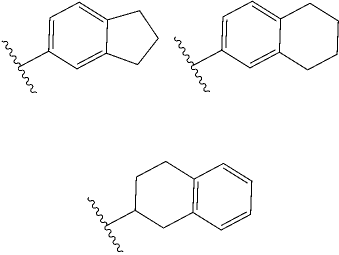

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems (e.g., fused, spiro, bridged bicyclic). A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “bicyclic heterocyclyl” refers to a fused, spiro, or bridged heterocyclyl group that contains two rings. Representative examples of a bicyclic heterocyclyl include, for example:

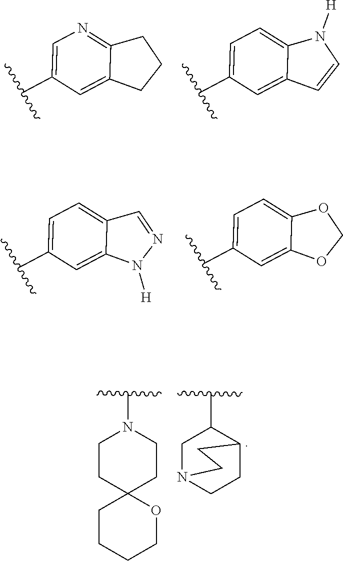

In certain embodiments, the bicyclic heterocyclyl is an carbocyclic ring fused to partially unsaturated heterocyclic ring, that together form a bicyclic ring structure having 8-10 ring atoms (e.g., where there are 1, 2, 3, or 4 heteroatoms selected from the group consisting of nitrogen, oxygen, and sulfur).

The term “heterocyclylene” refers to a diradical of a heterocyclyl group. An exemplary heterocyclylene group is

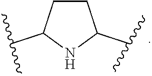

The heterocyclylene may contain, for example, 3-6 ring atom (i.e., a 3-6 membered heterocyclylene). In certain embodiments, the heterocyclylene is a 3-6 membered heterocyclylene containing 1, 2, or 3 three heteroatoms selected from the group consisting of oxygen, nitrogen, and sulfur.

The term “bicyclic heterocyclylene” refers to a diradical of a bicyclic heterocyclyl group.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

Any aryl (e.g., phenyl), cycloalkyl (e.g., C₃₋₇cycloalkyl), heterocyclyl (e.g., 3-7 membered heterocyclyl), heteroaryl (e.g., 5-6 membered heteroaryl) may be optionally substituted unless otherwise states. In some embodiments, Any aryl (e.g., phenyl), cycloalkyl (e.g., C₃₋₇cycloalkyl), heterocyclyl (e.g., 3-7 membered heterocyclyl), heteroaryl (e.g., 5-6 membered heteroaryl) may be optionally substituted with 1-4 substituents independently for each occurrence selected from the group consisting of halogen, C₁₋₆alkyl, C₁₋₆haloalkyl, C₁₋₆alkoxy, cyano, N(R^{aa})₂, —CH₂N(R^{aa})₂, and hydroxyl, wherein R^{aa} is independently for each occurrence hydrogen or C₁₋₆alkyl.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently alkoxy, aryloxy, alkyl, alkenyl, alkynyl, amide, amino, aryl, arylalkyl, carboxy, cyano, cycloalkyl, ester, ether, formyl, halogen, haloalkyl, heteroaryl, heterocyclyl, hydroxyl, ketone, nitro, sulfide, sulfonyl, or sulfonamide. Exemplary carbamates include arylcarbamates and heteroaryl carbamates, e.g., wherein at least one of R_{g}, Rₕ and Rᵢ are independently aryl or heteroaryl, such as phenyl and pyridinyl.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —RₐC(O)N(R_{b})—, —RₐC(O)N(R_{b})R_{c}—, —C(O)NR_{b}R_{c}, or —C(O)NH₂, wherein Rₐ, R_{b} and R_{c} are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro. The amide can be attached to another group through the carbon, the nitrogen, R^{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring.

The term “amidino” as used herein refers to a radical of the form —C(═NR)NR′R″ where R, R′, and R″ are each independently alkyl, alkenyl, alkynyl, amide, aryl, arylalkyl, cyano, cycloalkyl, haloalkyl, heteroaryl, heterocyclyl, hydroxyl, ketone, or nitro.

The term “alkanoyl” as used herein refers to a radical —O—CO-alkyl.

The term “oxo” is art-recognized and refers to a “═O” substituent. For example, a cyclopentane substituted with an oxo group is cyclopentanone.

The term “sulfonamide” or “sulfonamido” as used herein refers to a radical having the structure —N(Rᵣ)—S(O)₂—Rₛ— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, an optional substituent may be selected from the group consisting of: C₁₋₆alkyl, cyano, halogen, —O—C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂, wherein R^{a} is hydrogen or C₁₋₆alkyl. In some embodiments, an optional substituent may be selected from the group consisting of: C₁₋₆alkyl, halogen, —O—C₁₋₆alkyl, and —CH₂N(R^{a})₂, wherein R^{a} is hydrogen or C₁₋₆alkyl.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(+)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Abbreviations as used herein include diisopropylethylamine (DIPEA); dimethylformamide (DMF); methylene chloride (DCM); tetrahydrofuran (THF); trifluoroacetic acid (TFA); triethylamine (TEA); dimethylsulfoxide (DMSO); diisopropylethylamine (DIEA); ethyl acetate (EtOAc or EA); pentane (PE); 2,2′-bis(diphenylphosphino)-1,1′-binaphthyl (BINAP); p-methoxybenzyl (PMB); 2-(trimethylsilyl)ethoxymethyl (SEM); N-Bromosuccinimide (NBS); flash column chromatography (FCC); supercritical fluid chromatography (SFC); acetonitrile (ACN); acetic acid (AcOH); ammonium acetate (NH₄OAc); ethylene bridged hybrid (BEH); broadband inverse (BBI); cyclohexane (Cy); dichloroethane (DCE); dimethylamine (NHMe₂); dimethylcyclohexanedicarboxylate (DMCD); ethanol (EtOH); ethylene acetate (EA); high-performance liquid chromatography (HPLC); in situ chemical oxidation (ISCO); methanol (MeOH); methylmagnesium bromide (MeMgBr); mass spectrometry, electrospray (MS (ES)); methyl tert-butyl ether (MTBE); methyl iodide (MeI); nuclear magnetic resonance spectroscopy (NMR); [1,1′-Bis(diphenylphosphino)ferrocene]dichloropalladium(II), complex with dichloromethane (PdCl₂(dppf)-DCM); photodiode array (PDA); potassium acetate (KOAc); p-toluenesulfonic acid (p-TsOH); room temperature (RT); sodium acetate (NaOAc); sodium triacetoxyborohydride (NaBH(AcO)₃); solid phase extraction (SPE); thin layer chromatography (TLC); triethylamine (Et₃N); 2-(trimethylsilyl)ethoxymethyl chloride (SEMCl); and ultra performance liquid chromatography/mass spectrometry (UPLC/MS).

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₄, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Substituted Benzimidazole Carboxamides and Related Compounds

It has been discovered that the active site (binding site) of human acid ceramidase (ASAH-1), as determined by x-ray crystallography, contains a plurality of hydration sites, each of which is occupied by a single molecule of water and whose position and energetics (which incorporates the enthalpy, entropy, and free energy values associated with each water molecule) have been calculated. Each of these water molecules has a stability rating (namely, a numerical calculation which incorporates the enthalpy, entropy, and free energy values associated with each water molecule), which provides a measurable value associated with the relative stability of water molecules that occupy hydration sites in the binding pocket of the acid ceramidase enzyme. Water molecules occupying hydration sites in the binding pocket of acid ceramidase having a stability rating of >2.5 kcal/mol are referred to as unstable waters. It is contemplated that the displacement or disruption of an unstable water molecule (i.e., a water molecule having a stability rating of greater than 2.5 kcal/mol), or replacement of a stable water molecule (i.e., a water molecule having a stability rating of less than 1 kcal/mol), by an inhibitor results in tighter binding of that inhibitor. Accordingly, it is contemplated that an inhibitor designed to displace one or more unstable water molecules (namely, a water molecule having a stability rating of greater than 2.5 kcal/mol) may bind more tightly to the binding pocket and, therefore, will be a more potent inhibitor as compared to an inhibitor that does not displace unstable water molecules. Certain of the compounds described herein were designed to displace one or more unstable water molecules in the binding pocket.

### Compounds

One aspect of the invention provides substituted benzimidazole carboxamides and related compounds. The substituted benzimidazole carboxamides and related compounds are contemplated to be useful in the methods, compositions, and kits described herein. In certain embodiments, the substituted benzimidazole carboxamides or related compound is a compound embraced by formula (I):

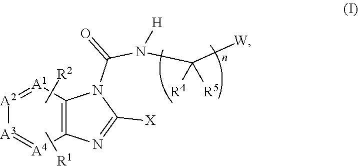

or a pharmaceutically acceptable salt thereof, wherein:

R¹ is selected from the group consisting of hydrogen, phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl);

R² is selected from the group consisting of hydrogen, halogen, C₁₋₆alkyl, C₁₋₆haloalkyl, —OR^{c}, and cyano;

R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene;

n is integer selected from 0 to 6;

X is selected from the group consisting of hydrogen, deuterium, —OR^{b}, —S(C₁₋₆alkyl), C₁₋₆alkyl, and phenyl;

R^{a} is independently, for each occurrence, hydrogen or C₁₋₆alkyl;

R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂;

R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;

W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₄alkynylene)-phenyl, and —(C₂₋₆alkynylene)-C₃₋₇cycloalkyl; and

A¹, A², A³, and A⁴ are CH, or one or two of A¹, A², A³, and A⁴ are N and the others are CH; and

wherein any aforementioned phenyl, C₃₋₇cycloalkyl, 3-10 membered heterocyclyl, —(C₂₋₆alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted; wherein, the compound is not the following:

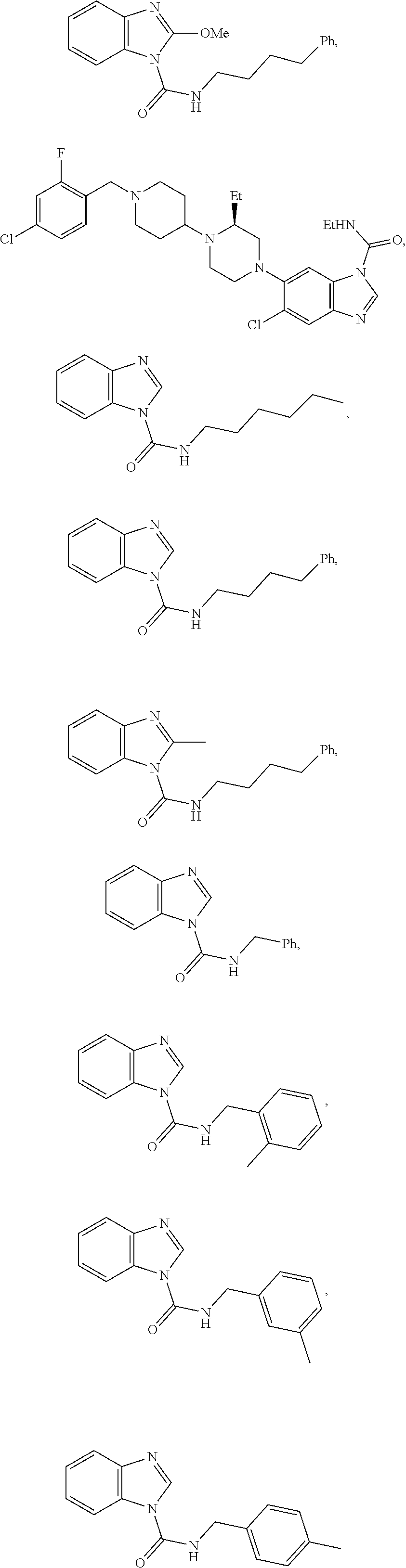

or a pharmaceutically acceptable salt thereof.

In certain embodiments, R¹ is selected from the group consisting of hydrogen, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl).

In certain embodiments, R¹ is selected from the group consisting of hydrogen, 3-10 membered heterocyclyl, 5-6 membered heteroaryl, C₁₋₆alkylene-(3-7 membered heterocyclyl), (3-7 membered heterocyclene)-(5-10 membered heteroaryl), (3-7 membered heterocyclene)-(C₃₋₇cycloalkyl), and (3-7 membered heterocyclene)-(3-7 membered heterocyclyl), wherein the 3-10 membered heterocyclyl, (3-7 membered heterocyclene)-(C₃₋₇cycloalkyl), and (3-7 membered heterocyclene)-(3-7 membered heterocyclyl) are optionally substituted with 1-3 substituents independently, for each occurrence, selected from the group consisting of halogen, cyano, oxo, C₁₋₆ alkyl, —C₁₋₆alkylene-CN, C₁₋₆haloalkyl, —O—C₁₋₆alkyl, —N(R^{a})₂, —C(O)—C₁₋₆alkyl, and —C₁₋₆alkylene-(C₃₋₇cycloalkyl).

In some embodiments, R¹ is selected from the group consisting of hydrogen,

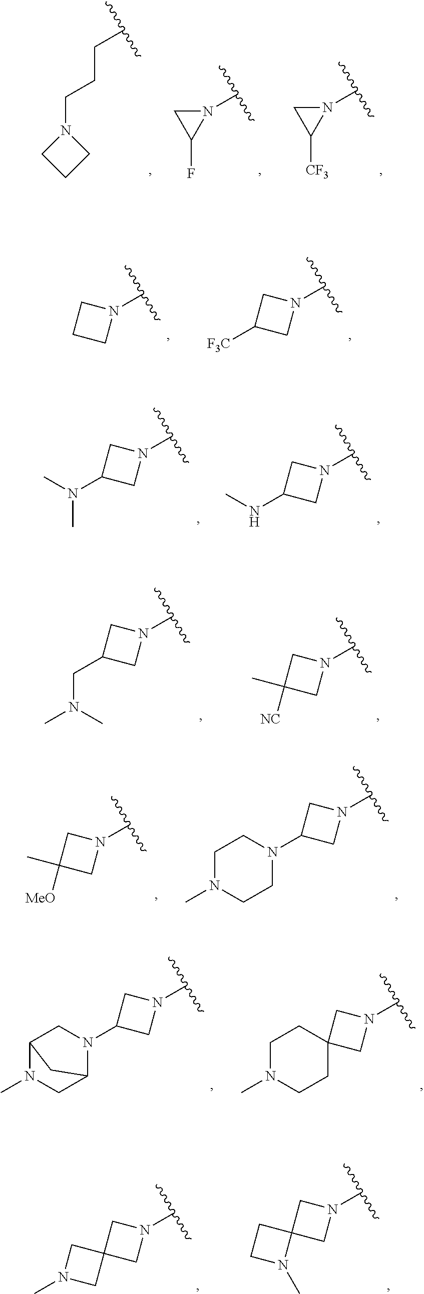

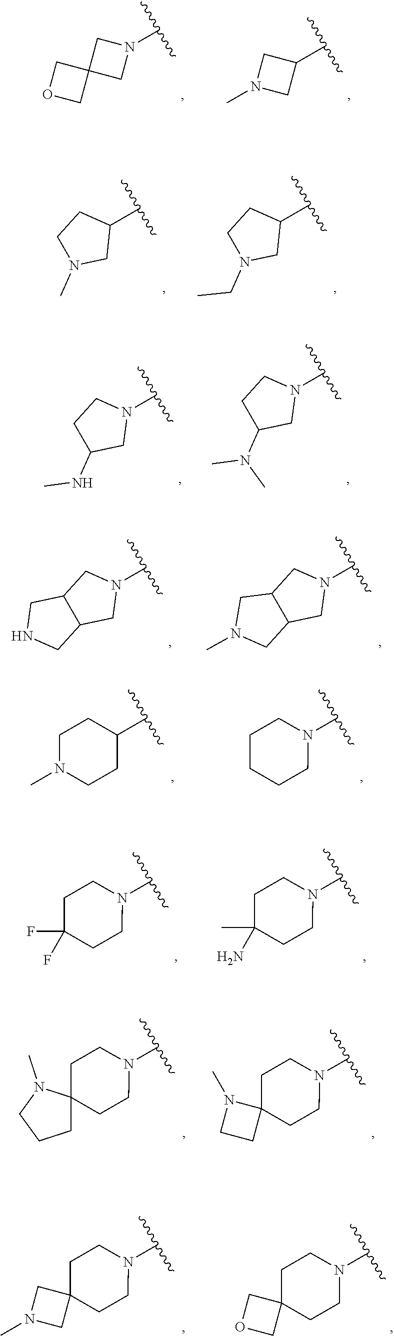

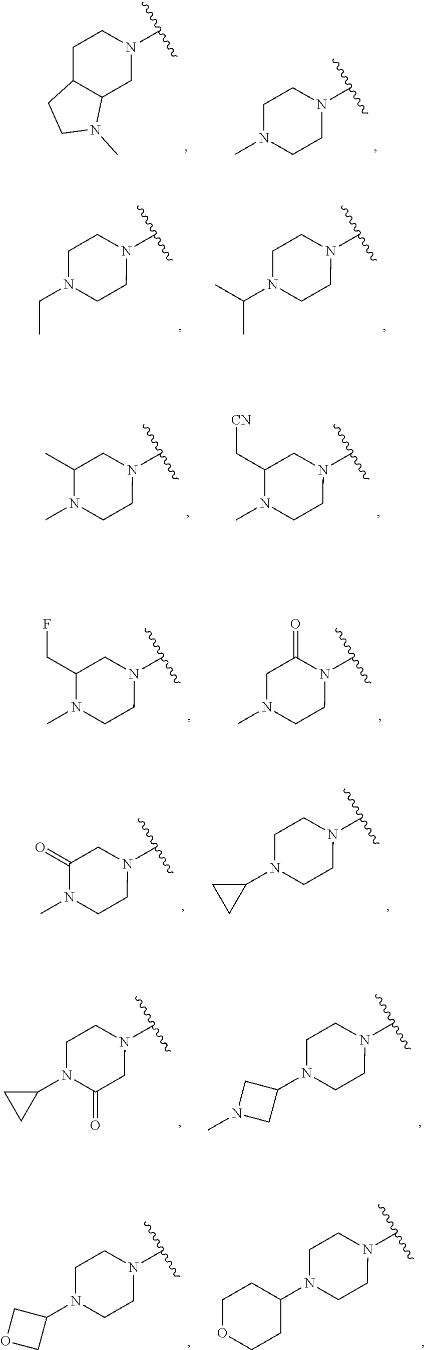

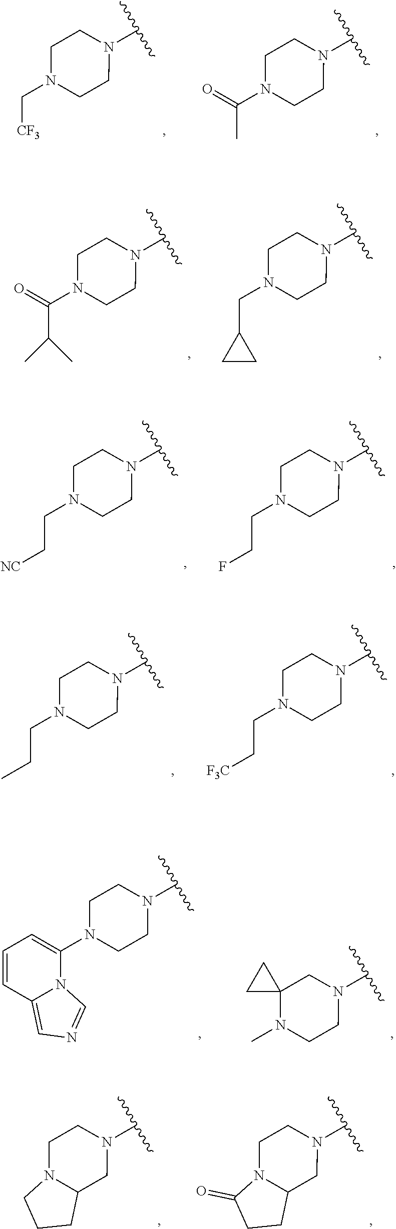

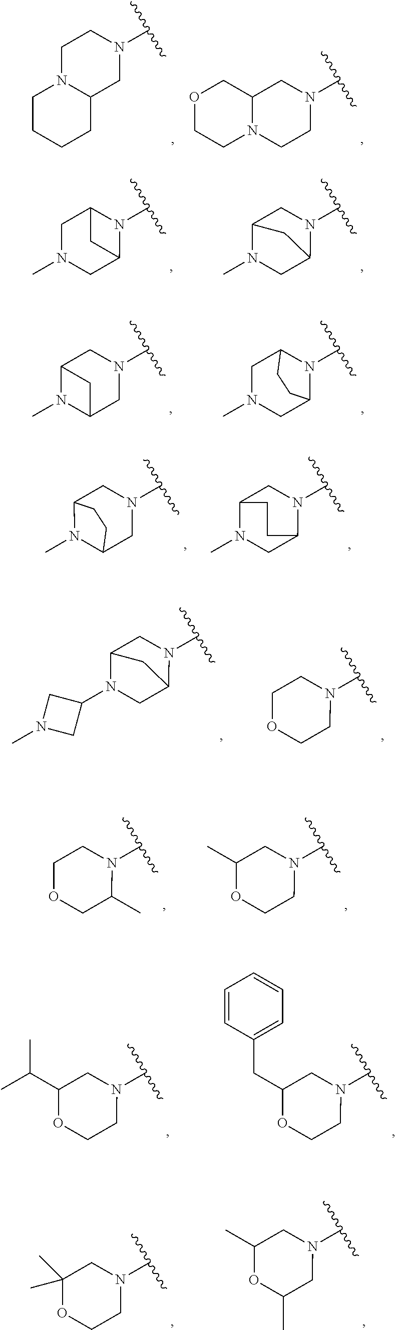

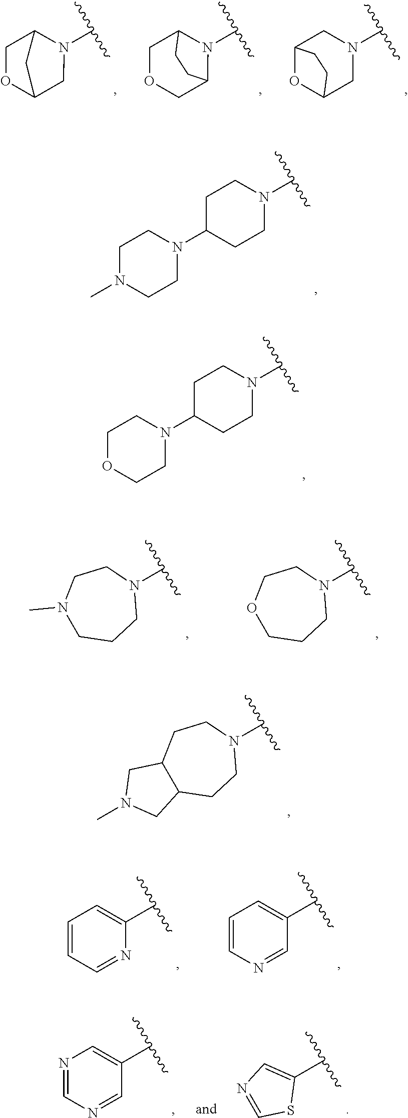

In some embodiments, R is

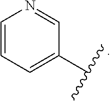 [SMILES: *c1cccnc1]

In some embodiments, R¹ is

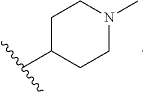 [SMILES: *C1CCN(C)CC1]

In some embodiments, R¹ is hydrogen.

In certain embodiments, R² is selected from the group consisting of hydrogen, halogen, C₁₋₄alkyl, C₁₋₄haloalkyl, —O—C₁₋₄alkyl, —O—C₁₋₄haloalkyl, O—C₃₋₇cycloalkyl, —O-(3-7 membered heterocyclyl), and cyano, wherein any aforementioned 3-7 membered heterocyclyl is optionally substituted.

In some embodiments, R² is selected from the group consisting of hydrogen, chlorine, fluorine, —CF₃, methoxy, methyl, cyano, —O—CF₃, —OCH₂CH₂N(CH₃)₂,

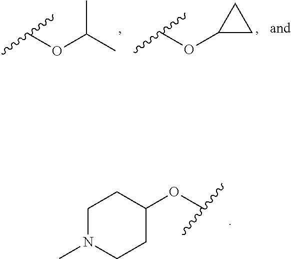

In some embodiments, R² is selected from the group consisting of hydrogen, chlorine, fluorine, —CF₃, methoxy, methyl, cyano, —O—CF₃,

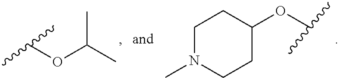

In some embodiments, R² is selected from the group consisting of hydrogen, chlorine, fluorine, —CF₃, methoxy, and methyl.

In some embodiments, R² is hydrogen.

In some embodiments, R² is methoxy.

In another aspect, provided herein is a compound of formula (I-a):

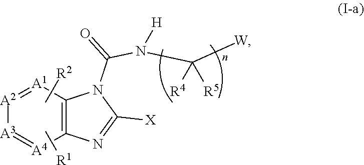

or a pharmaceutically acceptable salt thereof, wherein:

R¹ is selected from the group consisting of phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl);

R² is selected from the group consisting of hydrogen, halogen, C₁₋₆alkyl, C₁₋₆haloalkyl, —OR^{c}, and cyano;

R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene;

n is integer selected from 0 to 6;

X is selected from the group consisting of hydrogen, deuterium, —OR^{b}, —S(C₁₋₆alkyl), C₁₋₆ alkyl, and phenyl;

R^{a} is independently, for each occurrence, hydrogen or C₁₋₆alkyl;

R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂;

R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;

W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇ cycloalkyl; and

A¹, A², A³, and A⁴ are CH, or one or two of A¹, A², A³, and A⁴ are N and the others are CH;

wherein any aforementioned phenyl, C₃₋₇cycloalkyl, 3-10 membered heterocyclyl, —(C₂₋₆alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted;

wherein the compound is not a compound of

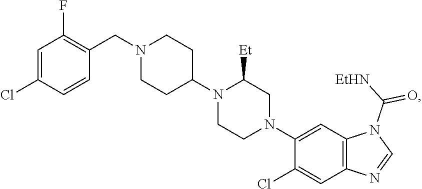

or a pharmaceutically acceptable salt thereof.

In certain embodiments, R¹ is selected from the group consisting of 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl).

In certain embodiments, R¹ is selected from the group consisting of 3-10 membered heterocyclyl, 5-6 membered heteroaryl, C₁₋₆alkylene-(3-7 membered heterocyclyl), (3-7 membered heterocyclene)-(5-10 membered heteroaryl), (3-7 membered heterocyclene)-(C₃₋₇cycloalkyl), and (3-7 membered heterocyclene)-(3-7 membered heterocyclyl), wherein the 3-10 membered heterocyclyl, (3-7 membered heterocyclene)-(C₃₋₇cycloalkyl), and (3-7 membered heterocyclene)-(3-7 membered heterocyclyl) are optionally substituted with 1-3 substituents independently, for each occurrence, selected from the group consisting of halogen, cyano, oxo, C₁₋₆ alkyl, —C₁₋₆alkylene-CN, C₁₋₆haloalkyl, —O—C₁₋₆alkyl, —N(R^{a})₂, —C(O)—C₁₋₆alkyl, and —C₁₋₆alkylene-(C₃₋₇cycloalkyl).

In some embodiments, R¹ is selected from the group consisting of

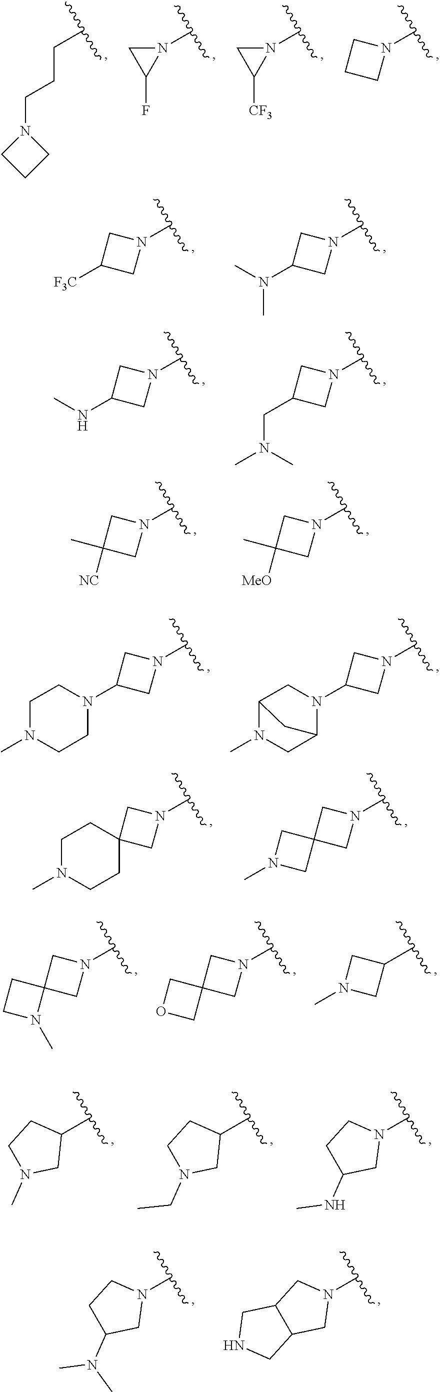

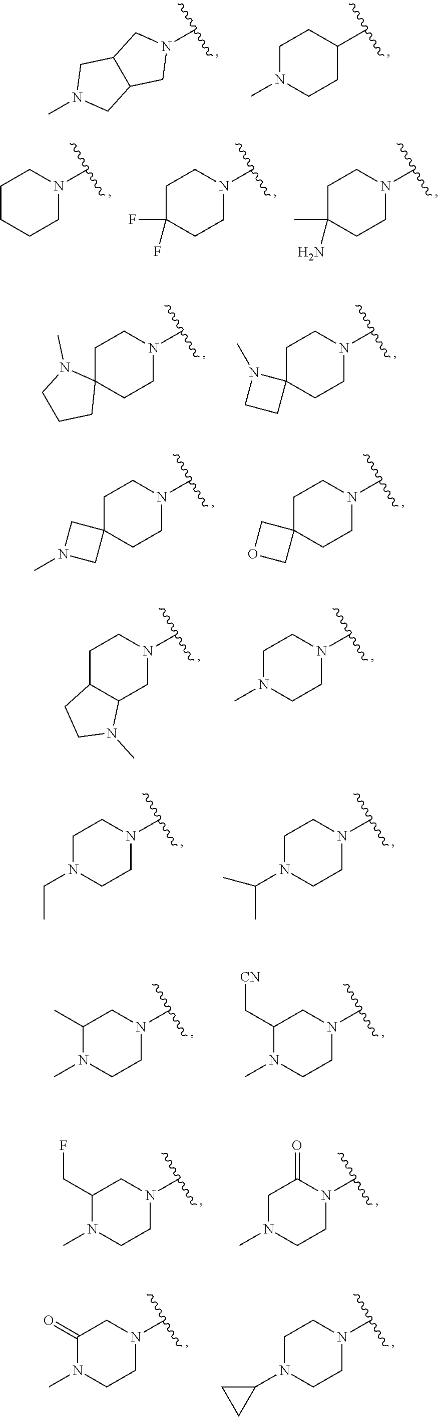

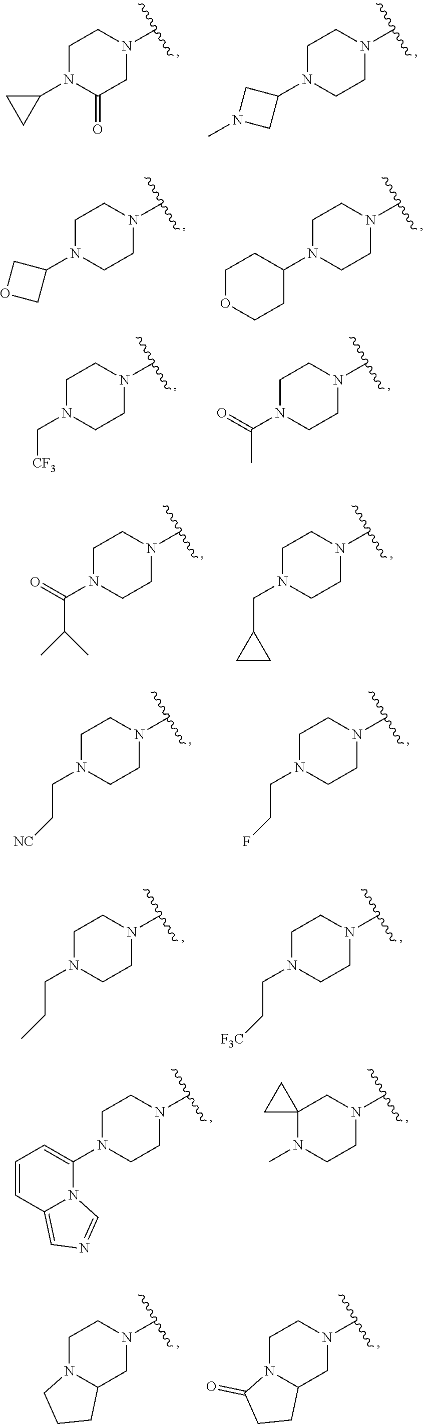

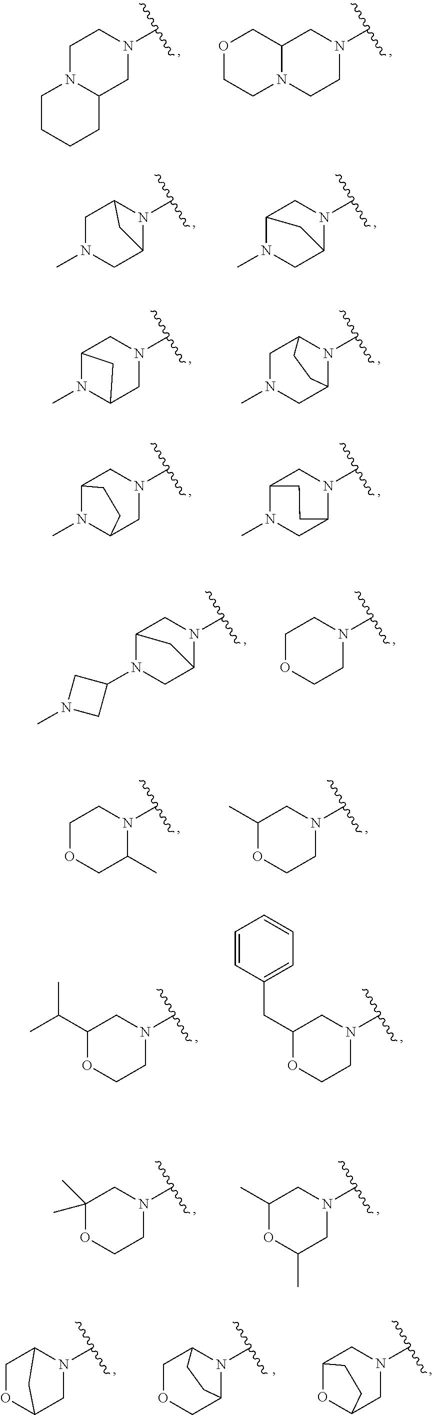

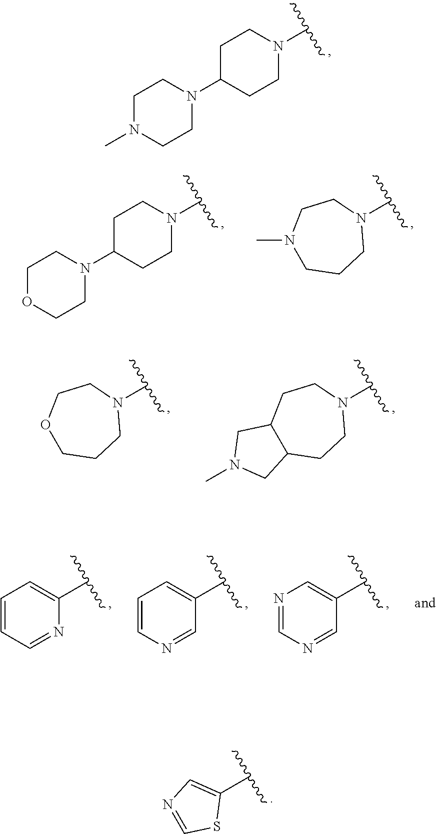

In some embodiments, R¹ is

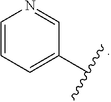 [SMILES: *c1cccnc1]

In some embodiments, R¹ is

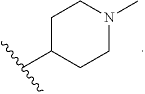 [SMILES: *C1CCN(C)CC1]

In certain embodiments, R² is selected from the group consisting of hydrogen, halogen, C₁₋₄alkyl, C₁₋₄haloalkyl, —O—C₁₋₄alkyl, and cyano.

In some embodiments, R² is selected from the group consisting of hydrogen, chlorine, fluorine, —CF₃, methoxy, methyl, and cyano.

In some embodiments, R² is selected from the group consisting of hydrogen, chlorine, fluorine, —CF₃, methoxy, and methyl.

In some embodiments, R² is hydrogen.

In some embodiments, R² is methoxy.

In another aspect, provided herein is a compound of formula (I-b):

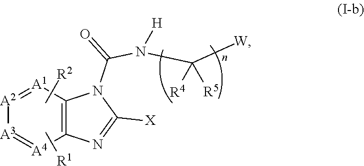

R¹ (I-b), or a pharmaceutically acceptable salt thereof, wherein:

R² is selected from the group consisting of halogen, C₁₋₆alkyl, C₁₋₆haloalkyl, —OR^{c}, and cyano; and

R¹ is selected from the group consisting of hydrogen, phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl);

R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene;

n is integer selected from 0 to 6;

X is selected from the group consisting of hydrogen, deuterium, —OR^{b}, —S(C₁₋₆alkyl), C₁₋₆alkyl, and phenyl;

R^{a} is independently, for each occurrence, hydrogen or C₁₋₆alkyl;

R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂;

R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;

W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl; and

A¹, A², A³, and A⁴ are CH, or one or two of A¹, A², A³, and A⁴ are N and the others are CH;

wherein any aforementioned phenyl, C₃₋₇cycloalkyl, 3-10 membered heterocyclyl, —(C₂₋₆alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted;

wherein the compound is not a compound of

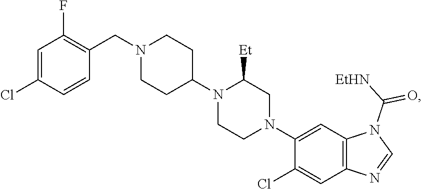

or a pharmaceutically acceptable salt thereof.

In certain embodiments, R¹ is selected from the group consisting of hydrogen, 3-10 membered heterocyclyl, 5-6 membered heteroaryl, and (3-7 membered heterocyclene)-(3-7 membered heterocyclyl).

In certain embodiments, R¹ is selected from the group consisting of hydrogen, 3-10 membered heterocyclyl, 5-6 membered heteroaryl, and (3-7 membered heterocyclene)-(3-7 membered heterocyclyl), wherein the 3-10 membered heterocyclyl and (3-7 membered heterocyclene)-(3-7 membered heterocyclyl) are optionally substituted with C₁₋₆alkyl.

In some embodiments, R¹ is selected from the group consisting of hydrogen,

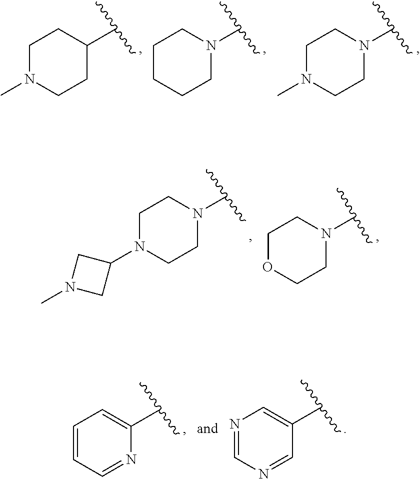

In some embodiments, R¹ is

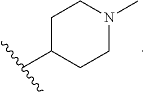 [SMILES: *C1CCN(C)CC1]

In some embodiments, R¹ is hydrogen.

In certain embodiments, R² is selected from the group consisting of halogen, C₁₋₄alkyl, C₁₋₄haloalkyl, —O—C₁₋₄alkyl, —O—C₁₋₄haloalkyl, —O-(3-7 membered heterocyclyl), and cyano, wherein any aforementioned 3-7 membered heterocyclyl is optionally substituted.

In some embodiments, R² is selected from the group consisting of chlorine, fluorine, —CF₃, methoxy, methyl, cyano, —O—CF₃,

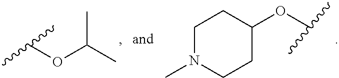

In some embodiments, R² is selected from the group consisting of chlorine, fluorine, —CF₃, methoxy, and methyl.

In some embodiments, R² is methoxy.

In each of the foregoing compounds of formula (I), (I-a) and (I-b), n is 0, 1, 2, 3, 4, 5, or 6. In each of the foregoing compounds of formula (I), (I-a) and (I-b), n is 1, 2, 3, or 4. In each of the foregoing compounds of formula (I), (I-a) and (I-b), n is 0. In each of the foregoing compounds of formula (I), (I-a) and (I-b), n is 1. In each of the foregoing compounds of formula (I), (I-a) and (I-b), n is 2. In each of the foregoing compounds of formula (I), (I-a) and (I-b), n is 3. In each of the foregoing compounds of formula (I), (I-a) and (I-b), n is 4. In each of the foregoing compounds of formula (I), (I-a) and (I-b), n is 5. In each of the foregoing compounds of formula (I), (I-a) and (I-b), n is 6.

In each of the foregoing compounds of formula (I), (I-a) and (I-b), W is selected from the group consisting of methyl, halogen, phenyl, 3-10 membered heterocyclyl, C₃₋₇cycloalkyl, —O—C₁₋₄alkyl, —O—C₁₋₄haloalkyl, —O-phenyl, —O—(C₁₋₄alkylene)-phenyl, 5-6 membered heteroaryl, C₂₋₆alkynylene, —(C₂₋₆alkynylene)-phenyl, and —(C₂₋₆alkynylene)-C₃₋₇cycloalkyl, wherein the phenyl, —O-phenyl, 5-6 membered heteroaryl, C₃₋₇cycloalkyl, C₂₋₆alkynylene, —(C₂₋₆alkynylene)-phenyl, —(C₂₋₆alkynylene)-C₃₋₇cycloalkyl, and 3-10 membered heterocyclyl, wherein the phenyl, C₃₋₇ cycloalkyl, 5-6 membered heteroaryl, C₂₋₆alkynylene, —(C₂₋₆alkynylene)-phenyl, and —(C₂₋₆alkynylene)-C₃₋₇cycloalkyl are optionally substituted with 1-3 substituents independently, for each occurrence, selected from the group consisting of fluorine, C₁₋₆alkyl, C₁₋₆haloalkyl, —O—C₁₋₆alkyl, —C₁₋₆alkylene-phenyl, and C₂₋₆alkynylene.

In each of the foregoing compounds of formula (I), (I-a) and (I-b), W is selected from the group consisting of methyl, fluorine, methoxy, —O—CF₃, phenyl, —O-phenyl,

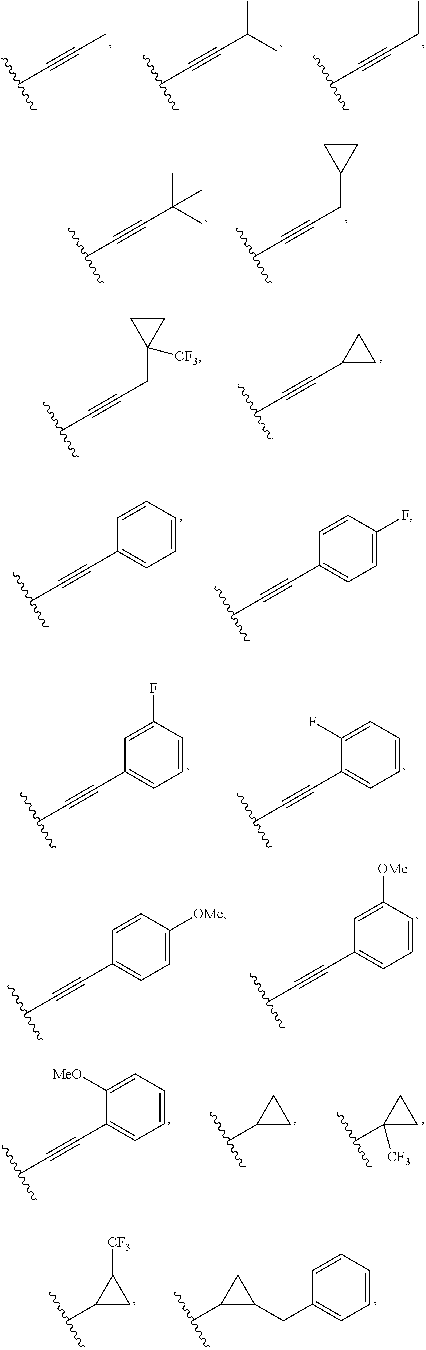

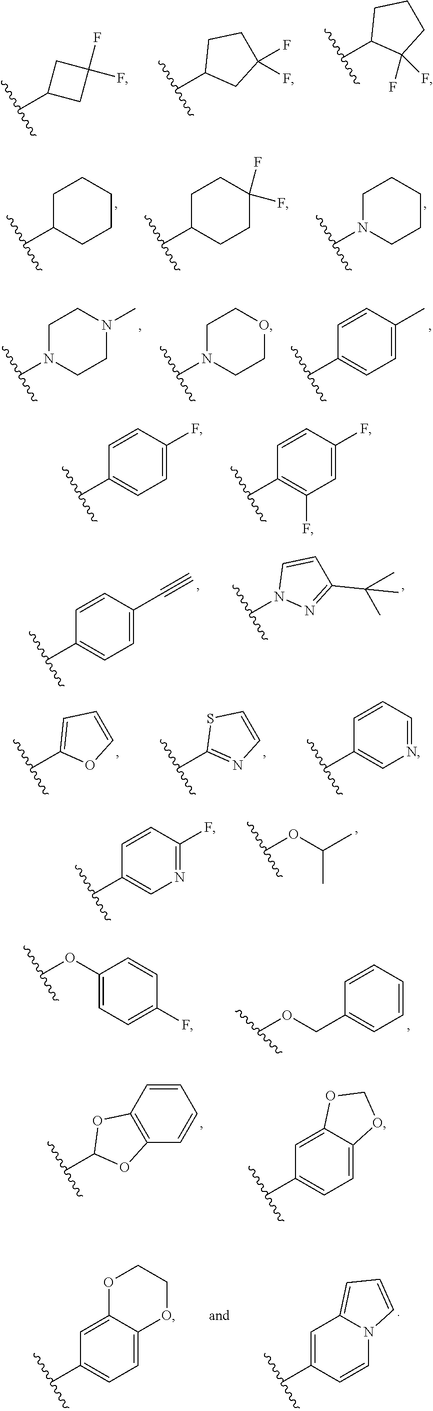

In each of the foregoing compounds of formula (I), (I-a) and (I-b), W is selected from the group consisting of methyl, fluorine, —O—CF₃, phenyl, —O-phenyl,

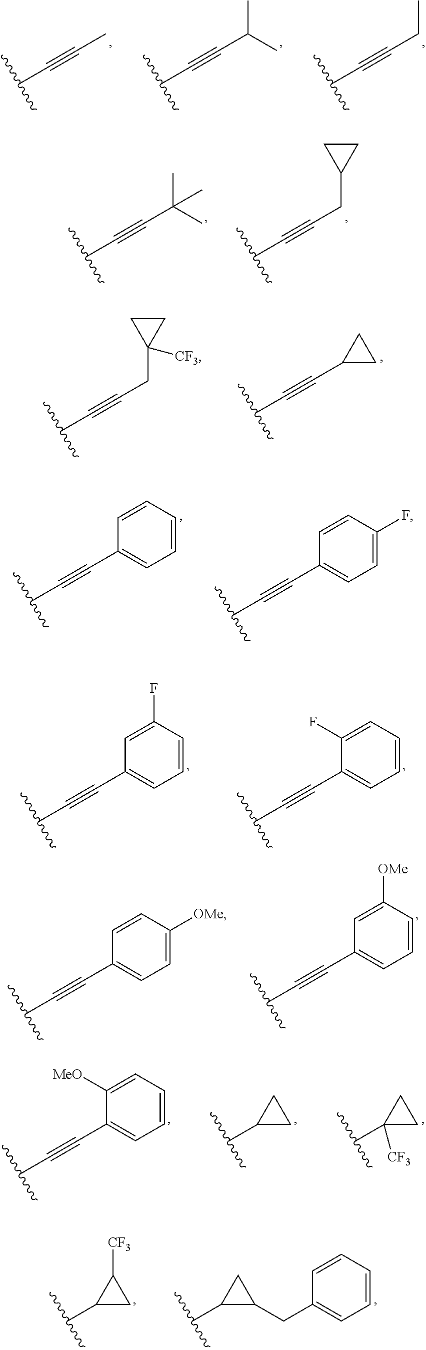

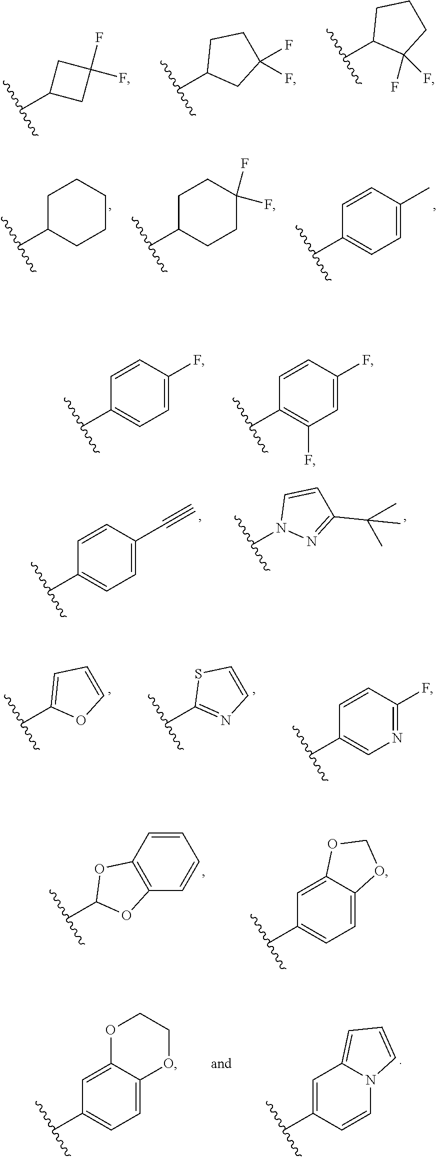

In each of the foregoing compounds of formula (I), (I-a) and (I-b), W is selected from the group consisting of methyl, phenyl, —O-phenyl,

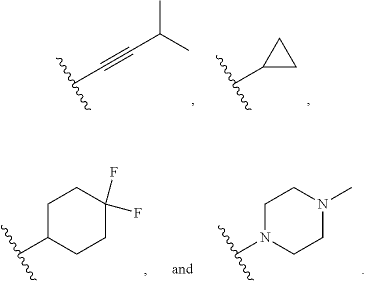

In each of the foregoing compounds of formula (I), (I-a) and (I-b), W is selected from the group consisting of methyl, phenyl, and —O-phenyl.

In each of the foregoing compounds of formula (I), (I-a) and (I-b), W is selected from methyl and phenyl.

In each of the foregoing compounds of formula (I), (I-a) and (I-b), W is phenyl. In some embodiments, W is methyl.

In another aspect, provided herein is a compound of formula (I-c):

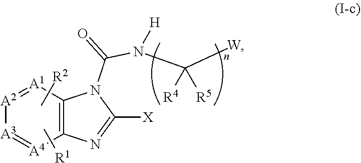

or a pharmaceutically acceptable salt thereof,

wherein:

W is phenyl; and n is integer selected from 0, 2, 3, 5, or 6, or

W is methyl; and n is integer selected from 0, 1, 2, 3, 4, or 6, or

W is selected from the group consisting of halogen, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl; and n is integer selected from 0, 1, 2, 3, 4, 5, or 6, and

R¹ is selected from the group consisting of hydrogen, phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl);

R² is selected from the group consisting of hydrogen, halogen, C₁₋₆alkyl, C₁₋₆haloalkyl, —OR^{c}, and cyano;

R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene;

X is selected from the group consisting of hydrogen, deuterium, —OR, —S(C₁₋₆alkyl), C₁₋₆alkyl, and phenyl;

R^{a} is independently, for each occurrence, hydrogen or C₁₋₆alkyl;

R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, and C₁₋₆alkylene-OR^{a}, C₁₋₆alkylene-N(R^{a})₂;

R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;

A¹, A², A³, and A⁴ are CH, or one or two of A¹, A², A³, and A⁴ are N and the others are CH; and

wherein any aforementioned phenyl, C₃₋₇cycloalkyl, 3-10 membered heterocyclyl, -(C₂₋₆ alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted.

In certain embodiments, R¹ is selected from the group consisting of hydrogen, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl).

In certain embodiments, R¹ is selected from the group consisting of hydrogen, 3-10 membered heterocyclyl, 5-6 membered heteroaryl, C₁₋₆alkylene-(3-7 membered heterocyclyl), (3-7 membered heterocyclene)-(5-10 membered heteroaryl), (3-7 membered heterocyclene)-(C₃₋₇cycloalkyl), and (3-7 membered heterocyclene)-(3-7 membered heterocyclyl), wherein the 3-10 membered heterocyclyl, (3-7 membered heterocyclene)-(C₃₋₇cycloalkyl), and (3-7 membered heterocyclene)-(3-7 membered heterocyclyl) are optionally substituted with 1-3 substituents independently, for each occurrence, selected from the group consisting of halogen, cyano, oxo, C t. 6alkyl, —C₁₋₆alkylene-CN, C₁₋₆haloalkyl, —O—C₁₋₆alkyl, —N(R′)₂, —C(O)—C₁₋₆alkyl, and —C₁₋₆alkylene-(C₃₋₇cycloalkyl).

In certain embodiments, R¹ is selected from the group consisting of hydrogen,

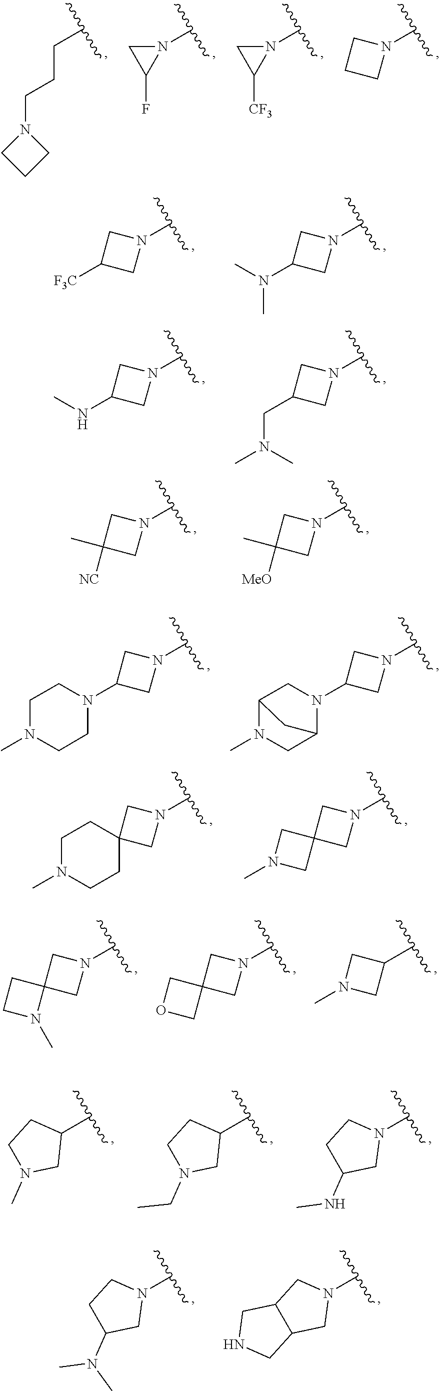

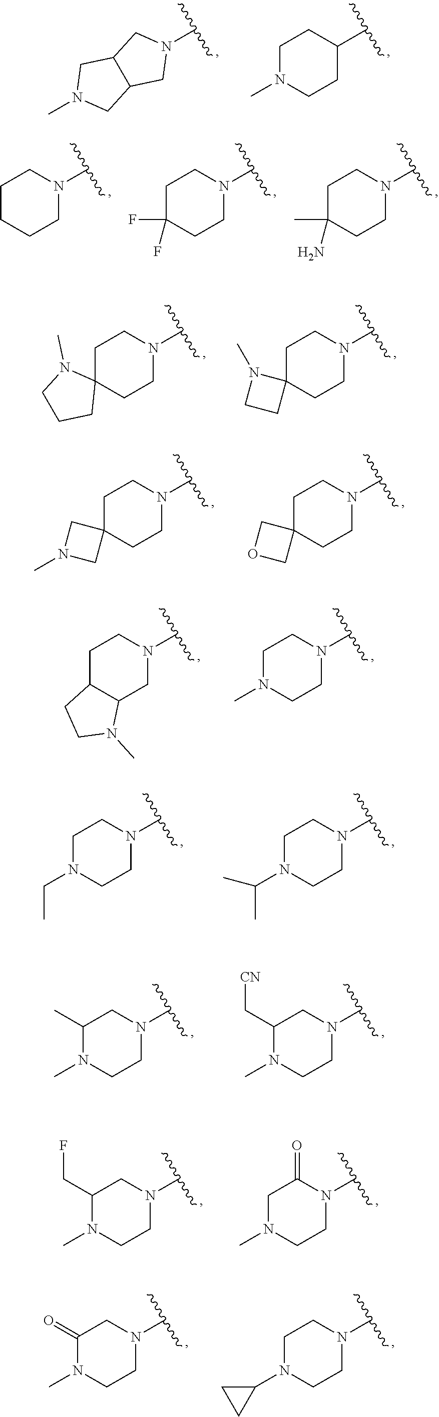

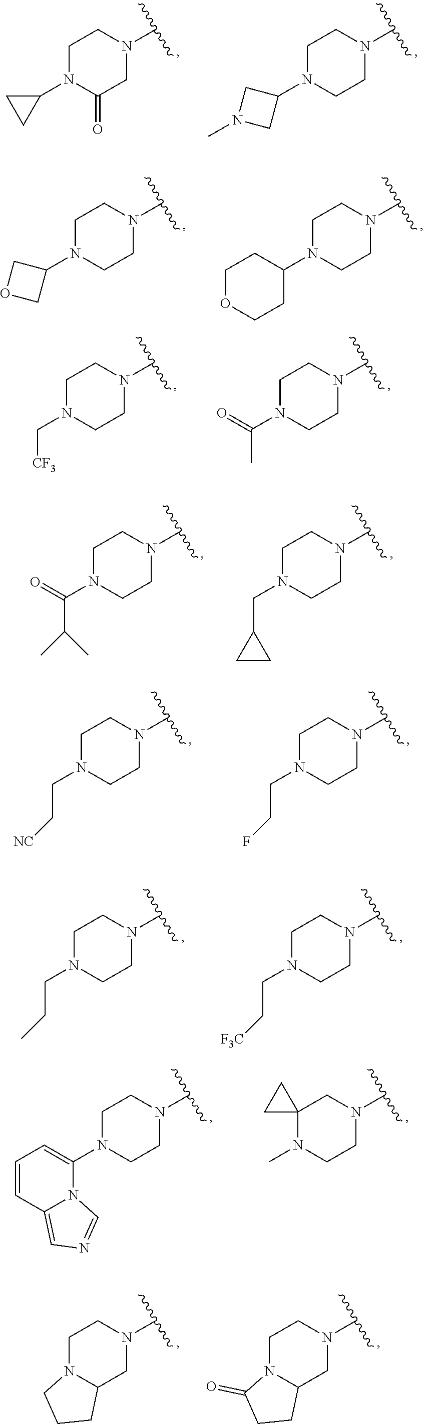

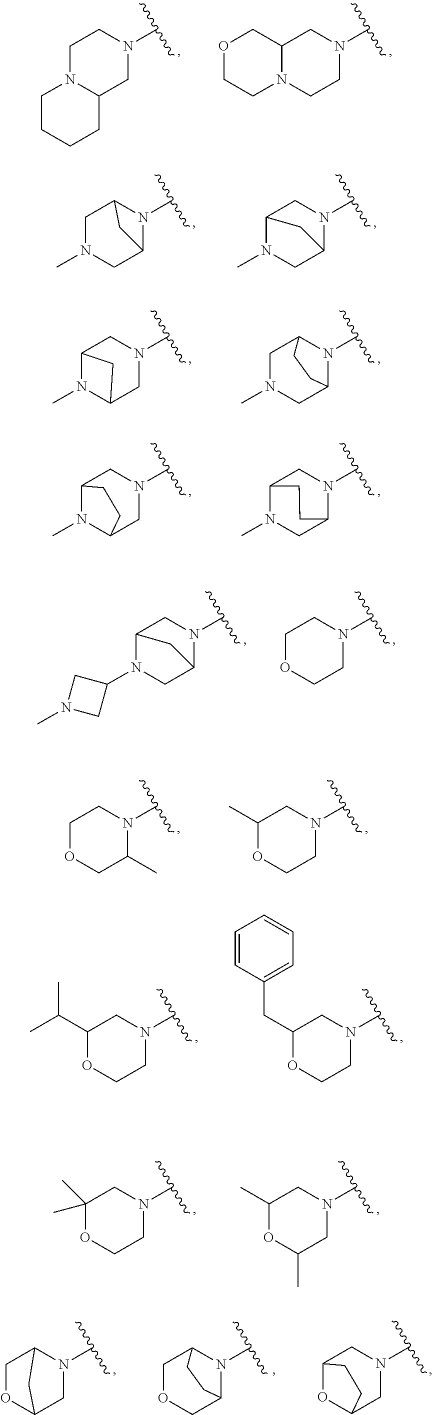

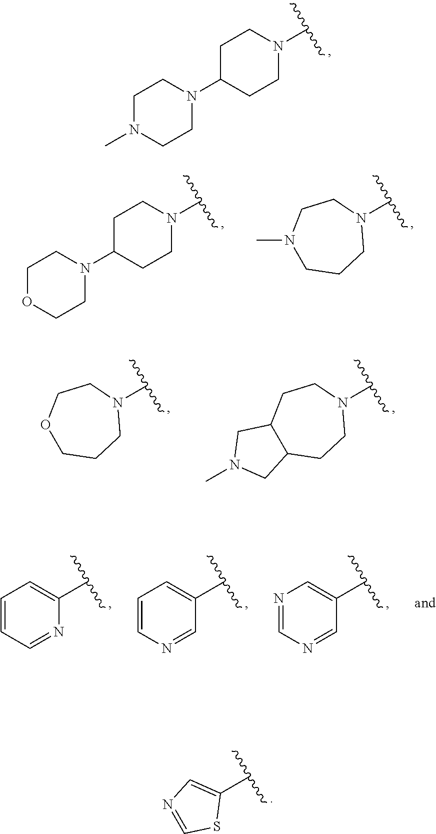

In some embodiments, R¹ is

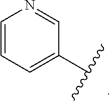 [SMILES: *c1cccnc1]

In some embodiments, R¹ is

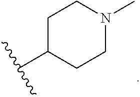 [SMILES: *C1CCN(C)CC1]

In some embodiments, R¹ is hydrogen.

In certain embodiments, R² is selected from the group consisting of hydrogen, halogen, C₁₋₄alkyl, C₁₋₄haloalkyl, —O—C₁₋₄alkyl, —O—C₁₋₄haloalkyl, —O—C₃₋₇cycloalkyl, —O-(3-7 membered heterocyclyl), and cyano, wherein any aforementioned 3-7 membered heterocyclyl is optionally substituted.

In some embodiments, R² is selected from the group consisting of hydrogen, chlorine, fluorine, —CF₃, methoxy, methyl, cyano, —O—CF₃,

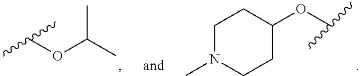

In some embodiments, R² is selected from the group consisting of hydrogen, chlorine, fluorine, —CF₃, methoxy, and methyl.

In some embodiments, R² is hydrogen. In some embodiments, R² is methoxy.

In certain embodiments, n is 0, 1, 2, 3, or 5 and W is selected from the group consisting of methyl, halogen, phenyl, 3-10 membered heterocyclyl, C₃₋₇cycloalkyl, —O—C₁₋₄alkyl, —O—C₁₋₄ haloalkyl, —O-phenyl, —O—(C₁₋₄alkylene)-phenyl, 5-6 membered heteroaryl, C₂₋₆alkynylene, —(C₂₋₆alkynylene)-phenyl, and —(C₂₋₆alkynylene)-C₃₋₇cycloalkyl, wherein the phenyl, —O-phenyl, 5-6 membered heteroaryl, C₃₋₇cycloalkyl, C₂₋₆alkynylene, —(C₂₋₆alkynylene)-phenyl, —(C₂₋₆alkynylene)-C₃₋₇cycloalkyl, and 3-10 membered heterocyclyl, wherein the phenyl, C₃₋₇cycloalkyl, 5-6 membered heteroaryl, C₂₋₆alkynylene, —(C₂₋₆alkynylene)-phenyl, and —(C₂₋₆alkynylene)-C₃₋₇cycloalkyl are optionally substituted with 1-3 substituents independently, for each occurrence, selected from the group consisting of fluorine, C₁₋₆alkyl, C₁₋₆haloalkyl, —O—C₁₋₆alkyl, —C₁₋₆alkylene-phenyl, and C₂₋₆alkynylene.

In some embodiments, n is 0, 1, 2, 3, or 5 and W is selected from the group consisting of methyl, fluorine, methoxy, —O—CF₃, phenyl, —O-phenyl,

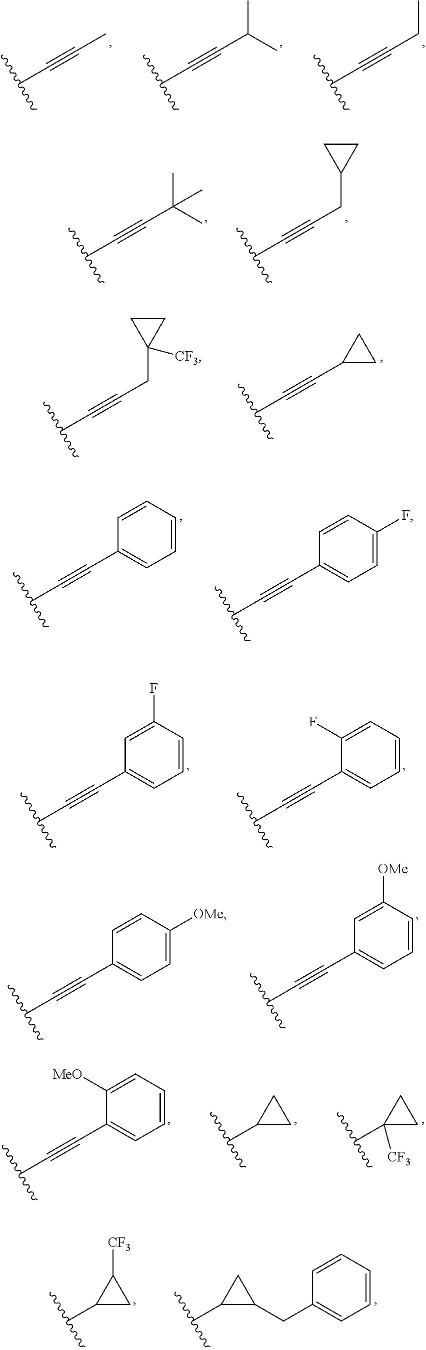

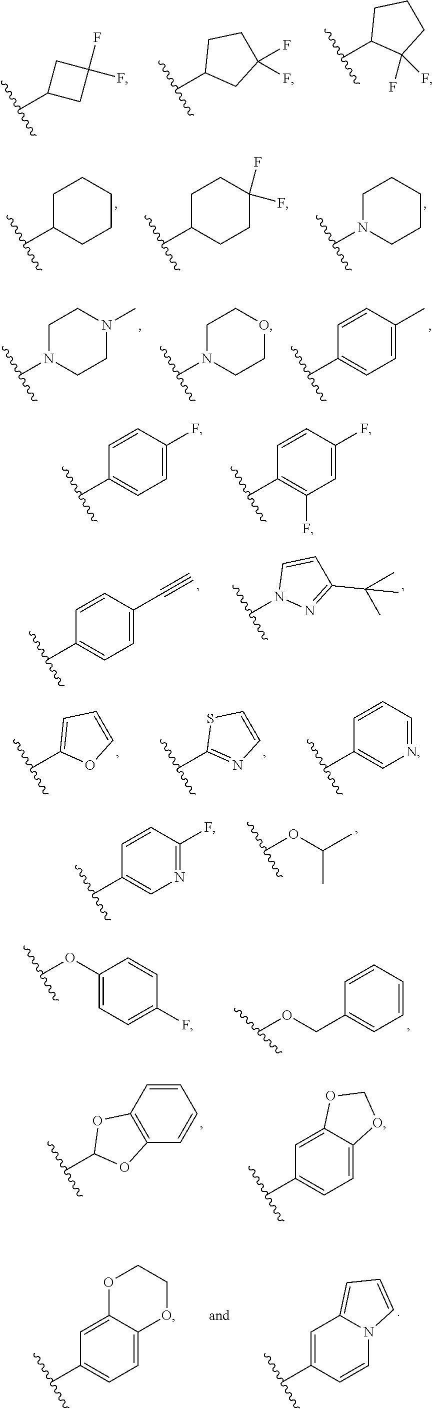

In some embodiments, n is 0, 1, 2, 3, or 5 and W is selected from the group consisting of methyl, phenyl, and —O-phenyl.

In some embodiments, n is 0, 1, 2, 3, or 5 and W is selected from methyl and phenyl.

In some embodiments, n is 0, 1, 2, 3, or 5 and W is phenyl. In some embodiments, n is 0, 1, 2, 3, or 5 and W is methyl.

In certain embodiments, n is 4 and W is selected from the group consisting of methyl, halogen, 3-10 membered heterocyclyl, C₃₋₇cycloalkyl, —O—C₁₋₄alkyl, —O—C₁₋₄haloalkyl, —O-phenyl, —O—(C₁₋₄alkylene)-phenyl, 5-6 membered heteroaryl, C₂₋₆alkynylene, —(C₂₋₆alkynylene)-phenyl, and —(C₂₋₆alkynylene)-C₃₋₇cycloalkyl, wherein the phenyl, —O-phenyl, 5-6 membered heteroaryl, C₃₋₇cycloalkyl, C₂₋₆alkynylene, —(C₂₋₆alkynylene)-phenyl, —(C₂₋₆alkynylene)-C₃₋₇cycloalkyl, and 3-membered heterocyclyl, wherein the phenyl, C₃₋₇cycloalkyl, 5-6 membered heteroaryl, C₂₋₆alkynylene, —(C₂₋₆alkynylene)-phenyl, and —(C₂₋₆alkynylene)-C₃₋₇cycloalkyl are optionally substituted with 1-3 substituents independently, for each occurrence, selected from the group consisting of fluorine, C₁₋₆alkyl, C₁₋₆haloalkyl, —O—C₁₋₆alkyl, —C₁₋₆alkylene-phenyl, and C₂₋₆alkynylene.

In some embodiments, n is 4 and W is selected from the group consisting of methyl,

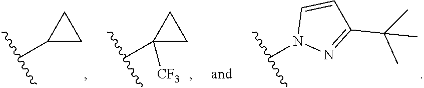

In some embodiments, n is 4 and W is methyl or —O-phenyl. In some embodiments, n is 4 and W is methyl.

In each of the foregoing compounds of formula (I), (I-a), (I-b), and (I-c), X is selected from the group consisting of hydrogen, deuterium, methyl, OR^{b}, and —SCH₃.

In each of the foregoing compounds of formula (I), (I-a), (I-b), and (I-c), X is selected from the group consisting of hydrogen, deuterium, methyl, methoxy,

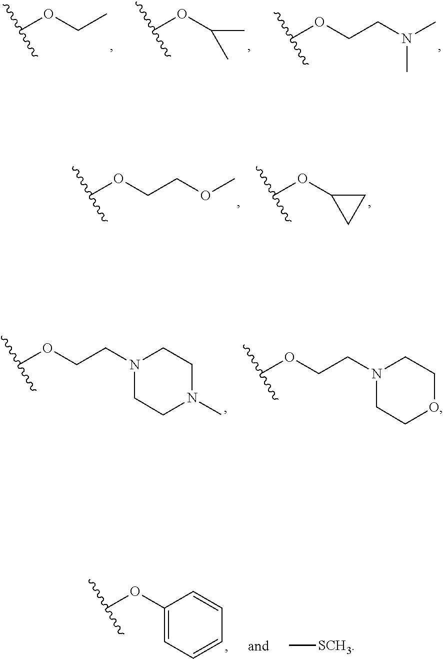

In each of the foregoing compounds of formula (I), (I-a), (I-b), and (I-c), X is

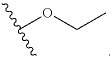 [SMILES: *OCC]

In each of the foregoing compounds of formula (I), (I-a), (I-b), and (I-c), R⁴ and R⁵ are independently, for each occurrence, selected from the group consisting of hydrogen, methyl, fluorine, and CF₃; or R⁴ and R⁵ can be taken together to form cyclopropyl.

In each of the foregoing compounds of formula (I), (I-a), (I-b), and (I-c), R⁴ and R⁵ are independently selected from hydrogen and methyl.

In each of the foregoing compounds of formula (I), (I-a), (I-b), and (I-c), R⁴ and R⁵ are hydrogen.

In each of the foregoing compounds of formula (I), (I-a), (I-b), and (I-c), A¹, A², A³, and A⁴ are CH. In some embodiments, A¹, A², and A³ are CH and A⁴ is N. In some embodiments, A¹, A², and A⁴ are CH and A³ is N. In some embodiments, A¹, A³, and A⁴ are CH and A² is N. In some embodiments, A², A³, and A⁴ are CH and A¹ is N.

In some embodiments, the compound is a compound of formula (I-d):

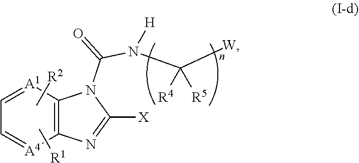

or a pharmaceutically acceptable salt thereof,
wherein R¹, R², R⁴, R⁵, A¹, A¹, X, n, and W are as defined herein, for example, as shown above in connection with formula (I).

In some embodiments, the compound is a compound of formula (I-e):

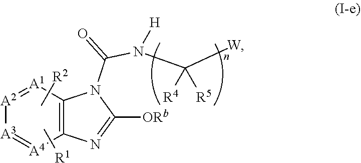

or a pharmaceutically acceptable salt thereof, wherein the variables are as defined herein, for example, as shown above in connection with formula (I).

In some embodiments, the compound is a compound of formula (I-f):

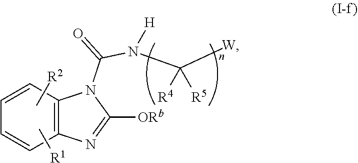

or a pharmaceutically acceptable salt thereof, wherein the variables are as defined herein, for example, as shown above in connection with formula (I).

In certain embodiments, the compound is a compound of formula (I-g):

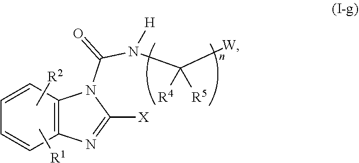

or a pharmaceutically acceptable salt thereof, wherein:

R¹ is selected from the group consisting of phenyl, C₃₋₇cycloalkyl, 3-10 membered heterocyclyl, 5-6 membered heteroaryl, (3-7 membered heterocyclene)-(5-10 membered heteroaryl), (3-7 membered heterocyclene)-(C₃₋₇ cycloalkyl), (3-7 membered heterocyclene)-(3-7 membered heterocyclyl), and (5-6 membered heteroarylene)-(3-7 membered heterocyclyl), wherein the phenyl, C₃₋₇cycloalkyl, 3-10 membered heterocyclyl, 5-6 membered heteroaryl, or (3-7 membered heterocyclene)-(3-7 membered heterocyclyl) is optionally substituted with one or more substituents independently, for each occurrence, selected from the group consisting of halogen, cyano, oxo, C₁₋₆alkyl, —C₁₋₄alkylene-CN, C₁₋₆haloalkyl, —O—C₁₋₆alkyl, —N(R^{a})₂, —C(O)—C₁₋₆alkyl, —C₁₋₆alkylene-unsubstituted phenyl, —C₁₋₆alkylene-N(R^{a})₂, and —C₁₋₆alkylene-(C₃₋₇cycloalkyl);

R² is selected from the group consisting of hydrogen, halogen, C₁₋₆alkyl, C₁₋₆haloalkyl, —OR^{c}, and cyano;

R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene, wherein the C₃₋₇cycloalkylene is optionally substituted;

n is integer selected from 0 to 6;

X is selected from the group consisting of hydrogen, deuterium, —OR^{b}, —S(C₁₋₆alkyl), C₁₋₆alkyl, and phenyl, wherein the phenyl is optionally substituted;

R^{a} is independently, for each occurrence, hydrogen or C₁₋₆alkyl;

R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂, wherein the phenyl, C₃₋₇cycloalkyl, 3-7 membered heterocyclyl, or 5-6 membered heteroaryl are optionally substituted;

R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂, wherein the phenyl, C₃₋₇cycloalkyl, 3-7 membered heterocyclyl, or 5-6 membered heteroaryl are optionally substituted; and

W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl, wherein the phenyl, C₃₋₇cycloalkyl, 3-10 membered heterocyclyl, —(C₂₋₆alkynylene)-phenyl, —(C₂₋₆alkynylene)-C₃₋₇cycloalkyl, or 5-6 membered heteroaryl is optionally substituted.

In some embodiments, the compound is a compound of formula (I-h):

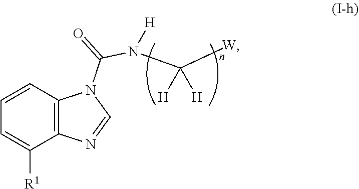

or a pharmaceutically acceptable salt thereof, wherein:

R¹ is 3-10 membered monocyclic or bicyclic heterocyclyl or (3-7 membered heterocyclene)-(3-7 membered heterocyclyl), wherein the 3-10 membered monocyclic or bicyclic heterocyclyl or (3-7 membered heterocyclene)-(3-7 membered heterocyclyl) is optionally substituted with C₁₋₆alkyl;

n is 2 or 3; and

W is phenyl, —O-phenyl, or —(C₂₋₆alkynylene)-phenyl.

In each of the foregoing compounds of formula (I), (I-a), (I-b), and (I-c), X is R¹ is selected from the group consisting of

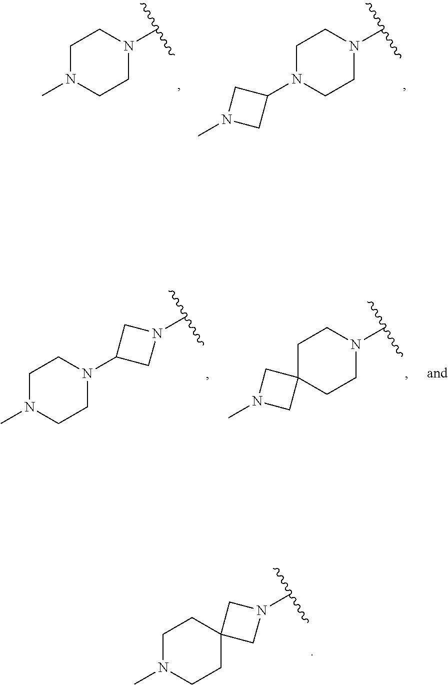

In some embodiments, n is 2. In other embodiments, n is 3.

In certain embodiments, W is selected from the group consisting of phenyl, —O-phenyl, and

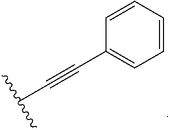 [SMILES: *C#Cc1ccccc1]

In another aspect, provided herein is a compound of formula (II):

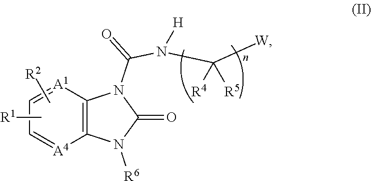

or a pharmaceutically acceptable salt thereof, wherein:

A¹ and A⁴ are independently selected from CH and N;

R¹ is selected from the group consisting of hydrogen, phenyl, C₃₋₇cycloalkyl, 3-7 membered heterocyclyl, 5-6 membered heteroaryl, C₁₋₆alkylene-N(R^{a})₂, (3-7 membered heterocyclene)-(3-7 membered heterocyclyl), (5-6 membered heteroarylene)-(3-7 membered heterocyclyl) and —C(O)-(3-7 membered heterocyclyl);

R² is selected from the group consisting of hydrogen, halogen, C₁₋₆alkyl, C₁₋₆haloalkyl, —OR^{c}, and cyano;

R⁴ and R⁵ are independently, for each occurrence, selected from the group consisting of hydrogen, C₁₋₆alkyl, C₁₋₆haloalkyl, and halogen;

R⁶ is hydrogen or C₁₋₂ alkyl;

n is integer between 0 to 6;

R^{a} is independently hydrogen or C₁₋₆alkyl;

R^{b} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;

R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(Rₐ)₂; and

W is selected from the group consisting of methyl, halogen, phenyl, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, and —(C₂₋₆alkynylene)-C₃₋₇cycloalkyl

wherein any aforementioned phenyl, 3-7 membered heterocyclyl, or 5-6 membered heteroaryl is optionally substituted,
wherein, the compound is not a compound of

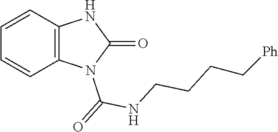 [SMILES: O=C(NCCCCc1ccccc1)n1c(=O)[nH]c2ccccc21]

or a pharmaceutically acceptable salt thereof.

In another aspect, provided herein is a compound of formula (II-a):

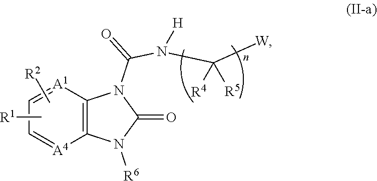

or a pharmaceutically acceptable salt thereof, wherein:

A¹ and A⁴ are independently selected from CH and N;

R¹ is selected from the group consisting of hydrogen, phenyl, C₃₋₇cycloalkyl, 3-7 membered heterocyclyl, 5-6 membered heteroaryl, C₁₋₆alkylene-N(R^{a})₂, (3-7 membered heterocyclene)-(3-7 membered heterocyclyl), (5-6 membered heteroarylene)-(3-7 membered heterocyclyl) and —C(O)-(3-7 membered heterocyclyl);

R² is selected from the group consisting of hydrogen, halogen, C₁₋₆alkyl, C₁₋₆haloalkyl, —OR^{c}, and cyano;

R⁴ and R⁵ are independently, for each occurrence, selected from the group consisting of hydrogen, C₁₋₆alkyl, C₁₋₆haloalkyl, and halogen;

R⁶ is hydrogen or C₁₋₂ alkyl;
R^{a} is independently hydrogen or C₁₋₆alkyl;

R^{b} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;

R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; and

W is selected from the group consisting of methyl, halogen, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, and —(C₂₋₆alkynylene)-C₃₋₇cycloalkyl, and n is integer selected from 0, 1, 2, 3, 4, 5, or 6, or

W is phenyl, and n is integer selected from 0, 1, 2, 3, 5, or 6, wherein any aforementioned phenyl, 3-7 membered heterocyclyl, or 5-6 membered heteroaryl is optionally substituted.

In some embodiments, A¹ and A⁴ are CH.

In some embodiments, A¹ is N and A⁴ is CH.

In some embodiments, A¹ is CH and A⁴ is N.

In some embodiments, R¹ is 5-6 membered heteroaryl or hydrogen.

In some embodiments, R¹ is selected from the group consisting of hydrogen,

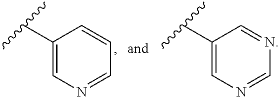 [SMILES: *c1cccnc1 ; *c1cncnc1]

In some embodiments, R¹ is hydrogen.

In some embodiments, R² is hydrogen.

In some embodiments, R⁴ and R⁵ are independently selected, at each occurrence, from hydrogen and methyl.

In some embodiments, R⁴ and R⁵ are hydrogen.

In some embodiments, R⁶ is selected from the group consisting of hydrogen, methyl, and ethyl.

In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, W is selected from the group consisting of methyl, phenyl, and 5-6 membered heteroaryl, wherein the 5-6 membered heteroaryl is optionally substituted.

In some embodiments, W is selected from the group consisting of methyl, phenyl, and

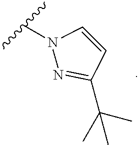 [SMILES: *n1ccc(C(C)(C)C)n1]

In certain embodiments, the compound is a compound described in the Examples, or a pharmaceutically acceptable salt thereof.

In certain other embodiments, the compound is one of the compounds listed in Table 1 or a pharmaceutically acceptable salt thereof.

### Methods of Preparing Compounds

Methods for preparing compounds described herein are illustrated in the following synthetic schemes. These schemes are given for the purpose of illustrating the invention and should not be regarded in any manner as limiting the scope or the spirit of the invention. Starting materials shown in the schemes can be obtained from commercial sources or can be prepared based on procedures described in the literature.

The synthetic route illustrated in Scheme 1 depicts an exemplary procedure for preparing substituted benzimidazole carboxamides. In the first step, compound A (wherein the variables are as described herein) is treated with 4-nitrophenyl chloroformate and triethylamine in DCM and the resulting intermediate is treated with amine B (wherein the variables are as described herein) to afford compound C (a compound of formula (I)).

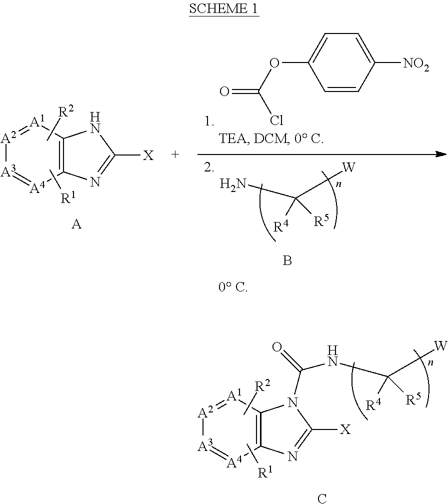

The synthetic route illustrated in Scheme 2 depicts another exemplary procedure for preparing substituted benzimidazole carboxamides. In the first step, compound A (wherein the variables (substituents such as R¹, R², etc.) are as described herein) is treated with 2,4-dinitrophenyl chloroformate and triethylamine in DCM and the resulting intermediate is treated with amine B (wherein the variables are as described herein) to afford compound C (a compound of formula (I)).

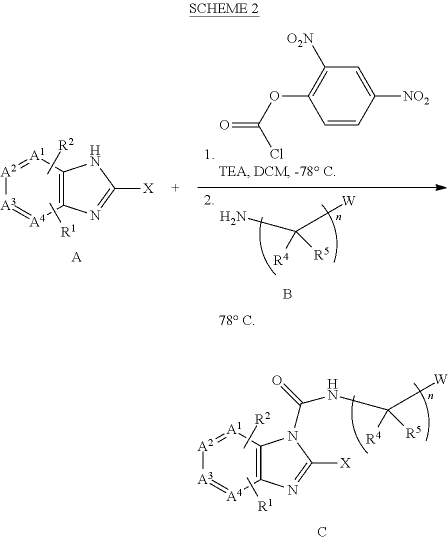

The synthetic route illustrated in Scheme 3 depicts another exemplary procedure for preparing substituted benzimidazole carboxamides. Compound A (wherein the variables are as described herein) is treated with isocyanate B (wherein the variables are as described herein) in the presence of triethylamine in DCM followed by reflux to afford compound C (a compound of formula (I)).

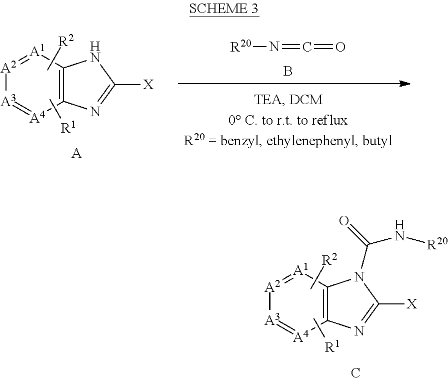

The synthetic route illustrated in Scheme 4 depicts an exemplary procedure for preparing compounds of formula (II). In the first step, compound A (wherein the variables are as described herein) is treated with 4-nitrophenyl chloroformate and triethylamine in DCM and the resulting intermediate is treated with amine B (wherein the variables are as described herein) to afford compound C (a compound of formula (II)).

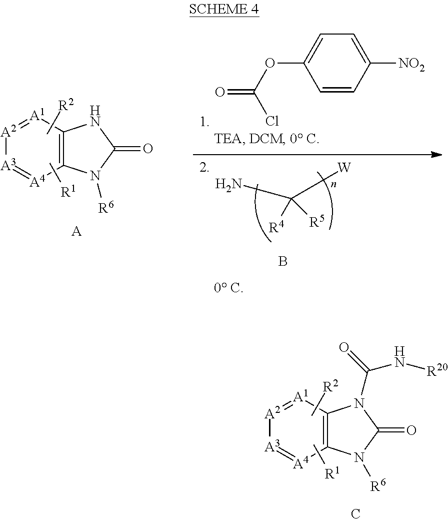

The reaction procedures in Schemes 1 to 3 are contemplated to be amenable to preparing a wide variety of substituted benzimidazole carboxamide compounds having different substituents. Furthermore, if a functional group that is part of the substituents would not be amenable to a reaction condition described in Schemes 1-3, it is contemplated that the functional group can first be protected using standard protecting group chemistry and strategies, and then the protecting group is removed after completing the desired synthetic transformation. See, for example, Greene, T. W.; Wuts, P. G. M. Protective Groups in Organic Synthesis, 2^{nd} ed.; Wiley: New York, 1991, for further description of protecting chemistry and strategies.

### III. Pharmaceutical Compositions

The invention provides pharmaceutical compositions comprising a compound described herein (e.g., a compound of Formula (I) or (II) (e.g., a compound of Formula (I-a), (I-b), (I-c), (I-d), (I-e), (I-f), (I-g), (I-h), or (II-a)) or related compound described herein. In certain embodiments, the pharmaceutical compositions preferably comprise a therapeutically-effective amount of one or more of a compound described herein, e.g., a compound of Formula (I) or (II) (e.g., a compound of Formula (I-a), (I-b), (I-c), (I-d), (I-e), (I-f), (I-g), (I-h), or (II-a)), formulated together with one or more pharmaceutically acceptable carriers. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention, e.g., a compound of Formula (I) or (II) (e.g., a compound of Formula (I-a), (I-b), (I-c), (I-d), (I-e), (I-f), (I-g), (I-h), or (II-a)).

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate: (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

### IV. Methods of Use

Sphingolipids are a family of membrane lipids derived from the aliphatic amino alcohol sphingosine and its related sphingoid bases. They are present in eukaryote membranes, where they exert important structural roles in the regulation of fluidity and subdomain structure of the lipid bilayer. In addition to serving roles in cell membrane structure and dynamics, sphingolipids also serve important signaling functions, for example, in the control of cell growth, cell differentiation, and cell death, and can be important for cell homeostasis and development. Zeidan et al. (2010) supra, Proksch et al. (2011) supra. Ceramide, a key member of this lipid class, has attracted attention in view of its impact on the replication and differentiation of neoplastic cells. Furuya et al. (2011).supra. For example, lower levels of ceramide have been discovered in several types of human tumors relative to normal tissue, where the level of ceramide appears to correlate inversely with the degree of malignant progression. Realini et al. (2013) supra.

Acid ceramidase is a cysteine amidase that catalyzes the hydrolysis of ceramide into sphingosine and fatty acid and is believed to be involved in the regulation of ceramide levels in cells and modulates the ability of this lipid messenger to influence the survival, growth and death of certain tumor cells. Id. Furthermore, acid ceramidase enzymes are abnormally expressed in various types of human cancer (e.g., prostate, head and neck, and colon) and serum AC levels are elevated in patients with melanoma relative to control subjects. Id

In addition, acid ceramidase enzymes have been implicated in a number of other disorders, including, inflammation (for example, rheumatoid arthritis and psoriasis), pain, inflammatory pain, and various pulmonary disorders. See, International Application Publication No. WO2015/173169. Furthermore, acid ceramidase enzymes have been identified as a target for the treatment of certain lysosomal storage disorders (for example, Gaucher's, Fabry's, Krabbe, Tay Sachs), and neurodegenerative disorders (for example, Alzheimer's, Parkinson's, Huntington's, and amyotrophic lateral sclerosis). See, International Application Publication Nos. WO2016/210116 and WO2016/210120.

It is contemplated that the compounds, compositions, and methods disclosed herein can be used to treat various disorders associated or correlated with elevated levels of acid ceramidase activity. The invention provides administering to a subject in need thereof an effective amount of a compound or composition disclosed herein, either alone or in a combination with another therapeutic agent to treat the disorder.

In certain embodiments, the compound or composition used in one or more of the methods described herein is one of the generic or specific compounds described in Section II, such as a compound of Formula (I), a compound embraced by one of the further embodiments describing definitions for certain variables of Formula (I), a compound of Formula (I-a), (I-b), (I-c), (I-d), (I-e), (I-f), (I-g), or (I-h), or a compound embraced by one of the further embodiments describing definitions for certain variables of Formula (I-a), (I-b), (I-c), (I-d), (I-e), (I-f), (I-g), or (I-h). In certain other embodiments, the compound is a compound of Formula (II) or a compound embraced by one of the further embodiments describing definitions for certain variables of Formula (II).

In certain embodiments, a method or composition described herein, is administered in combination with one or more additional therapies, e.g., surgery, radiation therapy, or administration of another therapeutic preparation. In certain embodiments, the additional therapy may include an additional therapeutic agent. The invention embraces combination therapy, which includes the administration of a compound described herein, e.g., a compound of Formula (I) or (ID) (e.g., a compound of Formula (I-a), (I-b), (I-c), (I-d), (I-e), (I-f), (I-g), (I-h), or (II-a)) or composition described herein and a second treatment and/or agent as part of a specific treatment regimen intended to provide the beneficial effect from the co-action of the foregoing. The beneficial effect of the combination may include pharmacokinetic or pharmacodynamic co-action resulting from the foregoing combination of agents and/or treatments.

The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

I. Cancer, Inflammation and Other Disorders

The compositions and methods disclosed herein can be used to treat various disorders associated or otherwise correlated with elevated levels of acid ceramidase activity. Exemplary disorders include cancer, inflammation, pain and inflammatory pain, or a pulmonary disease.

In certain embodiments, the compositions and methods disclosed herein can be used to treat cancer or inhibit cancer growth in a subject in need thereof. The invention provides a method of treating a cancer in a subject. The method comprises administering to the subject an effective amount of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition disclosed herein, either alone or in a combination with another therapeutic agent to treat the cancer in the subject.

Exemplary cancers include, but are not limited to, pre-malignant conditions, for example hyperplasia, metaplasia or dysplasia, cancer metastasis, benign tumors, angiogenesis, hyperproliferative disorders and benign dysproliferative disorders. The treatment may be prophylactic or therapeutic. The subject to be treated may be human or a non-human animal (e.g., a non-human primate or a non-human mammal).

In certain embodiments, a compound disclosed herein, e.g., a compound of Formula (I) or (II) (e.g., a compound of Formula (I-a), (I-b), (I-c), (I-d), (I-e), (I-f), (I-g), (I-h), or (II-a)), or a pharmaceutical composition containing such a compound, can be used to treat a disorder involving primary and/or metastatic neoplastic disease.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma)

In certain embodiments, the present invention provides a compound disclosed herein, e.g., a compound of Formula (I) or (II) (e.g., a compound of Formula (I-a), (I-b), (I-c), (I-d), (I-e), (I-f), (I-g), (I-h), or (II-a)), or a pharmaceutical composition disclosed herein for the use in the treatment and/or prevention of brain cancer, breast cancer, colon cancer, head and neck cancer, liver cancer, lung cancer (e.g., alveolar cancer), pancreatic cancer, prostate cancer, skin cancer (e.g., melanoma).

It is contemplated that the compounds disclosed can be used in combination with other treatments and/or therapeutic agents. The invention embraces combination therapy, which includes the administration of a compound described herein, e.g., a compound of Formula (I) or (II) (e.g., a compound of Formula (I-a), (I-b), (I-c), (I-d), (I-e), (I-f), (I-g), (I-h), or (II-a)), or related compound described herein and a second treatment and/or agent as part of a specific treatment regimen intended to provide the beneficial effect from the co-action of these therapeutic agents. The beneficial effect of the combination may include pharmacokinetic or pharmacodynamic co-action resulting from the combination of therapeutic agents.

In certain embodiments, a compound or pharmaceutical composition described herein, is administered in combination with one or more additional cancer therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a combination of therapeutics of different classes.

In certain embodiments, a method or pharmaceutical composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody-based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

In certain embodiments, a compound or pharmaceutical composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 BL. Exemplary CTLA-4 antibodies include ipilimumab or tremelimumab.

Exemplary cytotoxic agents that can be administered in combination with a compound or pharmaceutical composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a compound or pharmaceutical composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

In certain embodiments, a compound disclosed herein, e.g., a compound of Formula (I) or (II) (e.g., a compound of Formula (I-a), (I-b), (I-c), (I-d), (I-e), (I-f), (I-g), (I-h), or (II-a)), or a pharmaceutical composition containing such a compound, can be used to treat an inflammatory condition, such as rheumatoid arthritis and ulcerative cholitis. The invention provides a method of treating an inflammatory condition. The method comprises administering to the subject an effective amount of a compound (e.g., a compound of Formula (I), or (II) (e.g., a compound of Formula (I-a), (I-b), (I-c), (I-d), (I-e), (I-f), (I-g), (I-h), or (II-a)) or a pharmaceutical composition disclosed herein, either alone or in a combination with another therapeutic agent to treat the inflammatory condition in the subject.

As used herein, an inflammatory condition is a disease or condition characterized, in whole or in part, by inflammation or an inflammatory response in the patient. Typically, one or more of the symptoms of the inflammatory disease or condition is caused or exacerbated by an inappropriate, misregulated, or overactive inflammatory response. Inflammatory diseases or conditions may be chronic or acute. In certain embodiments, the inflammatory disease or condition is an autoimmune disorder.

Inflammatory conditions treatable using a compound or pharmaceutical composition disclosed herein may be characterized, for example, based on the primary tissue affected, the mechanism of action underlying the condition, or the portion of the immune system that is misregulated or overactive. Examples of inflammatory conditions, as well categories of diseases and conditions are provided herein. In certain embodiments, examples of inflammatory conditions that may be treated include inflammation of the lungs, joints, connective tissue, eyes, nose, bowel, kidney, liver, skin, central nervous system, vascular system, heart, or adipose tissue. In certain embodiments, inflammatory conditions which may be treated include inflammation due to the infiltration of leukocytes or other immune effector cells into affected tissue. In certain embodiments, inflammatory conditions which may be treated include inflammation mediated by IgE antibodies. Other relevant examples of inflammatory conditions which may be treated by the present disclosure include inflammation caused by infectious agents, including but not limited to viruses, bacteria, fungi, and parasites. In certain embodiments, the inflammatory condition that is treated is an allergic reaction. In certain embodiments, the inflammatory condition is an autoimmune disease.

Inflammatory lung conditions include asthma, adult respiratory distress syndrome, bronchitis, pulmonary inflammation, pulmonary fibrosis, and cystic fibrosis (which may additionally or alternatively involve the gastro-intestinal tract or other tissue(s)). Inflammatory joint conditions include rheumatoid arthritis, rheumatoid spondylitis, juvenile rheumatoid arthritis, osteoarthritis, gouty arthritis and other arthritic conditions. Inflammatory eye conditions include uveitis (including iritis), conjunctivitis, scleritis, and keratoconjunctivitis sicca. Inflammatory bowel conditions include Crohn's disease, ulcerative colitis, inflammatory bowel disease, and distal proctitis. Inflammatory skin conditions include conditions associated with cell proliferation, such as psoriasis, eczema, and dermatitis (e.g., eczematous dermatitides, topic and seborrheic dermatitis, allergic or irritant contact dermatitis, eczema craquelee, photoallergic dermatitis, phototoxicdermatitis, phytophotodermatitis, radiation dermatitis, and stasis dermatitis). Inflammatory conditions of the endocrine system include, but are not limited to, autoimmune thyroiditis (Hashimoto's disease), Type I diabetes, inflammation in liver and adipose tissue associated with Type II diabetes, and acute and chronic inflammation of the adrenal cortex. Inflammatory conditions of the cardiovascular system include, but are not limited to, coronary infarct damage, peripheral vascular disease, myocarditis, vasculitis, revascularization of stenosis, atherosclerosis, and vascular disease associated with Type II diabetes. Inflammatory conditions of the kidney include, but are not limited to, glomerulonephritis, interstitial nephritis, lupus nephritis, nephritis secondary to Wegener's disease, acute renal failure secondary to acute nephritis, Goodpasture's syndrome, post-obstructive syndrome and tubular ischemia. Inflammatory conditions of the liver include, but are not limited to, hepatitis (arising from viral infection, autoimmune responses, drug treatments, toxins, environmental agents, or as a secondary consequence of a primary disorder), obesity, biliary atresia, primary biliary cirrhosis and primary sclerosing cholangitis. In certain embodiments, the inflammatory condition is an autoimmune disease, for example, rheumatoid arthritis, lupus, alopecia, autoimmune pancreatitis, Celiac disease, Behcet's disease, Cushing syndrome, and Grave's disease. In certain embodiments, the inflammatory condition is a rheumatoid disorder, for example, rheumatoid arthritis, juvenile arthritis, bursitis, spondylitis, gout, scleroderma, Still's disease, and vasculitis.

In certain embodiments, the present invention provides a compound disclosed herein, e.g., a compound of Formula (I) or (II) (e.g., a compound of Formula (I-a), (I-b), (I-c), (I-d), (I-e), (I-f), (I-g), or (I-h), or (II-a)), or a pharmaceutical composition containing a compound disclosed herein for use in the treatment of a pain syndrome, disorder, disease or condition characterized by nociceptive pain, neuropathic pain, inflammatory pain, non-inflammatory pain, pain associated with acute conditions such as post-operative or post-traumatic stress disorders, pain associated with chronic conditions such as diabetes. The invention provides a method of treating pain. The method comprises administering to the subject an effective amount of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition disclosed herein, either alone or in a combination with another therapeutic agent to treat the pain in the subject.

A compound or composition described herein can be useful for the treatment (including prevention and/or alleviation) of chronic and/or acute pain, in particular non-inflammatory musculoskeletal pain such as back pain, fibromyalgia and myofascial pain, more particularly for reduction of the associated muscular hyperalgesia or muscular allodynia. Non-limiting examples of types of pain that can be treated by a compound or composition disclosed includes chronic conditions such as musculoskeletal pain, including fibromyalgia, myofascial pain, back pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, CPS (chronic pain syndrome), central pain, neuropathic pain such as trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiency or alcoholism; and acute pain such as pain after injuries, postoperative pain, pain during acute gout or pain during operations, such as jaw surgery.

In certain embodiments, the present invention provides a compound disclosed herein, e.g., a compound of Formula (I) or (II) (e.g., a compound of Formula (I-a), (I-b), (I-c), (I-d), (I-e), (I-f), (I-g), (I-h), or (II-a)), or a pharmaceutical composition disclosed herein for use in the treatment of a pulmonary disease, such as asthma, chronic obstructive pulmonary disease (COPD), adult respiratory disease, acute respiratory distress syndrome, chronic bronchitis, and emphysema. The invention provides a method of treating a pulmonary disease. The method comprises administering to the subject an effective amount of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition disclosed herein, either alone or in a combination with another therapeutic agent to treat the pulmonary disease in the subject.

II. Lysosomal Storage Disorders

Lysosomal storage disorders (LSDs) are a group of more than 50 clinically-recognized, rare inherited metabolic disorders that result from defects in lysosomal function (Walkley, J. (2009) INHERIT. METAB. DIS., 32(2): 181-9). LSDs are caused by dysfunction of the cell's lysosomes, which are heterogeneous subcellular organelles containing specific hydrolases that allow targeted processing or degradation of proteins, nucleic acids, carbohydrates, and lipids (HARRISON'S PRINCIPLES OF INTERNAL MEDICINE, 16^{th} Edition, vol. II, Chapter 20, pp. 2315-2319). The lysosome encloses an acidic environment and contains enzymes that catalyze the hydrolysis of biological macromolecules.

Individually, LSDs occur with incidences of less than 1:100,000, however, as a group the incidence is as high as 1 in 1,500 to 7,000 live births (Staretz-Chacham, et al. (2009) PEDIATRICS, 123(4): 1191-207). LSDs typically are caused by inborn genetic errors. Affected individuals generally appear normal at birth, however the diseases are progressive. The development of clinical disease may not occur until years or decades later, but is typically fatal.

It is believed that sphingosine-containing analogs (for example, glucosylsphingosine, galactosphingosine, lactosylsphingosine, GB3-sphingosine, and GM2-sphingosine) may accumulate in cells of subjects with certain lysosomal storage disorders or LSDs (for example, Gauchers disease, Krabbe disease, multiple sclerosis, Fabry's disease, and Tay Sachs disease, respectively) and that the accumulation of these sphingosine-containing analogs may contribute to the disease phenotype. See, e.g., International Application Publication No. WO2016/210116. Given that such sphingosine-containing analogs are often produced by acid ceramidase enzymes in the lysosomal compartments of cells in subjects with LSDs, the accumulation of the sphingosine-containing analogs to detrimental levels can be prevented or reduced by the use of an effective amount of one or more of the acid ceramidase inhibitors described herein.

In certain embodiments, a compound (e.g., a compound of Formula (I) or (II) (e.g., a compound of Formula (I-a), (I-b), (I-c), (I-d), (I-e), (I-f), (I-g), (I-h), or (II-a)) or pharmaceutical composition containing a compound disclosed herein can be used to treat a LSD in a subject in need thereof. The invention provides a method of treating an LSD in a subject. The method comprises administering to the subject an effective amount of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition disclosed herein, either alone or in a combination with another therapeutic agent to treat the LSD in the subject.

Exemplary LSDs include, for example, Krabbe disease, Fabry disease, Tay-Sachs disease, Sandhoff Variant A, or B, Pompe disease, Hunter's syndrome, Niemann Pick disease Types A and B, and Gaucher's disease.

It is contemplated that the compounds disclosed can be used in combination with other treatments and/or therapeutic agents. The invention embraces combination therapy, which includes the administration of a compound described herein, e.g., a compound of Formula (I) or (II)(e.g., a compound of Formula (I-a), (I-b), (I-c), (I-d), (I-e), (I-f), (I-g), (I-h), or (II-a)), or related compound described herein and a second treatment and/or agent as part of a specific treatment regimen intended to provide the beneficial effect from the co-action of these therapeutic agents. Exemplary second agents for use in treating Gaucher disease include, for example, imiglucerase (CEREZYME®), taliglucerase alfa (ELELYSO®), velaglucerase alfa (VPRIV®), eliglustat (CERDELGA®), and miglustat (ZAVESCA®) or a glucocerebrosidase activator such as one or more of the compounds described in International Application Publication No. WO2012/078855. Exemplary second agents for use in treating Fabry disease include, for example, alpha-galactosidase A (FABRAZYME®). Additional acid ceramidase inhibitors for use in combination therapies include, for example, those described in International Patent Application Publications WO 2015/173168 and WO 2015/173169, each of which are hereby incorporated by reference.

II. Neurodegenerative Disorders

Neurodegenerative disorders often are associated with reduction in the mass and/or volume of the brain, which may be due to the atrophy and/or death of brain cells, which are far more profound than those in a healthy subject that are attributable to aging. Neurodegenerative disorders can evolve gradually, after a long period of normal brain function, due to progressive degeneration (e.g., nerve cell dysfunction and death) of specific brain regions. Alternatively, neurodegenerative disorders can have a quick onset, such as those associated with trauma or toxins. The actual onset of brain degeneration may precede clinical expression by many years.

Examples of neurodegenerative disorders include, for example, Alzheimer's disease, Parkinson's disease, Huntington's disease, amyotrophic lateral sclerosis (ALS; also known as Lou Gehrig's disease or motor neuron disease), multiple sclerosis, and diffuse Lewy body disease. Once clinical expression occurs, the neurodegenerative disorder may be associated with impairment of motor function, for example, as observed in subjects with Parkinson's disease, Huntington's disease multiple sclerosis, or ALS. Alternatively or in addition, neurodegenerative disorders may be associated with cognitive impairment and/or the loss of cognitive function, for example, as observed in subjects with Alzheimer's disease.

Alzheimer's disease is a central nervous system (CNS) disorder that results in memory loss, unusual behavior, personality changes, and a decline in thinking abilities. These losses are related to the death of specific types of brain cells and the breakdown of connections and their supporting network (e.g., glial cells) between them. The earliest symptoms include loss of recent memory, faulty judgment, and changes in personality. Parkinson's disease is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles. Huntington's disease is another neurodegenerative disease that causes uncontrolled movements, loss of intellectual faculties, and emotional disturbance.

It has been observed that subjects with certain mutant alleles in genes encoding β-glucocerebrosidase activity (the GBA gene; Aharon-Peretz (2004) NEW. ENG. J. MED. 351: 1972-1977; Gan-Or et al. (2008) NEUROLOGY 70:2277-2283; Gan-Or et al. (2015) NEUROLOGY 3:880-887) and sphinomyelinase activity (the SMPD1 gene, Gan-Or et al. (2013) NEUROLOGY 80:1606-1610) have been associated with, and identified as a risk factor for, Parkinson's Disease. As a result defects with, or deficiencies in the activities of these enzymes, as in the case of Gaucher's disease and Niemann Pick types A and B, can cause an accumulation of glucosylceramide and sphingomyelin, which can then be converted to glucosylsphingosine or lyso-sphingomyelin, respectively, via acid ceramidase activity. The accumulation of glucosylsphingosine or lyso-sphingomyelin may thus be implicated in the development of Parkinson's disease. It is contemplated that the administration of an acid ceramidase inhibitor, which slows down, stops or reverses the accumulation of glucosylsphingosine and/or lyso-sphingomyelin can be used to treat Parkinson's Disease. For example, an acid ceramidase inhibitor can be used to improve motor and/or memory impairments symptomatic of Parkinson's disease.

Similarly, it has been observed that lactosylceramide (LacCer) is upregulated in the central nervous system of mice during chronic experimental autoimmune encephalomyelitis (EAE), a model of multiple sclerosis (Lior et al. (2014) NATURE MEDICINE 20:1147-1156). It is contemplated that the increase in LacCer may also result in an increase in lactosylsphingosine (LacSph) via conversion by an acid ceramidase (a lactosylceramide to lactosylsphingosine converting enzyme). Given the accumulation of lactosylsphingosine to a toxic or otherwise detrimental level or concentration in the lysosomal compartments of cells in subjects with multiple sclerosis, it is contemplated that the administration of an acid ceramidase inhibitor can reduce the accumulation of lactosylsphingosine thereby treating multiple sclerosis, which includes ameliorating a symptom associated with multiple sclerosis.

It has been observed that the level and activity of acid ceramidase can be elevated in subjects with Alzheimer's disease (Huang et al. (2004) EUROPEAN J. NEUROSCI. 20:3489-3497). Given that the accumulation of sphingosine or sphingosine analogs to a toxic or otherwise detrimental level or concentration in the lysosomal compartments of cells in subjects with Alzheimer's disease, it is contemplated that the administration of an acid ceramidase inhibitor can reduce the accumulation of the sphingosine or sphingosine analogs thereby treating Alzheimer's disease, which includes ameliorating a symptom associated with Alzheimer's disease.

Furthermore, given that a number of the foregoing neurodegenerative disorders, for example, Alzheimer's disease, are associated with a level of cognitive impairment and/or some decrease or loss of cognitive function, it is contemplated that the administration of an effective of an acid ceramidase inhibitor to a subject in need thereof may be reduce, stabilize, or reverse cognitive impairment and/or the loss of cognitive function. Cognitive function generally refers to the mental processes by which one becomes aware of, perceives, or comprehends ideas. Cognitive function involves all aspects of perception, thinking, learning, reasoning, memory, awareness, and capacity for judgment. Cognitive impairment generally refers to conditions or symptoms involving problems with thought processes. This may manifest itself in one or more symptoms indicating a decrease in cognitive function, such as impairment or decrease of higher reasoning skills, forgetfulness, impairments to memory, learning disabilities, concentration difficulties, decreased intelligence, and other reductions in mental functions.

Cognitive function and cognitive impairment may be readily evaluated using tests well known in the art. Performance in these tests can be compared over time to determine whether a treated subject is improving or whether further decline has stopped or slowed, relative to the previous rate of decline of that patient or compared to an average rate of decline. Tests of cognitive function, including memory and learning for evaluating human patients are well known in the art and regularly used to evaluate and monitor subjects having or suspected of having cognitive disorders such as Alzheimer's disease including the clock-drawing test (Agrell & Dehlin (1998) AGE & AGING 27:399-403). Even in healthy individuals, these and other standard tests of cognitive function can be readily used to evaluate beneficial affects over time.

In certain embodiments, a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition containing a compound disclosed herein can be used to treat a neurodegenerative disorder in a subject in need thereof. The invention provides a method of treating a neurodegenerative disorder in a subject. The method comprises administering to the subject an effective amount of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition disclosed herein, either alone or in a combination with another therapeutic agent to treat the neurodegenerative disorder in the subject.

Exemplary neurodegenerative disorders include, for example, Alzheimer's disease, Parkinson's disease, Huntington's disease, amyotrophic lateral sclerosis, Lewy body disease, dementia (e.g., frontotemporal dementia), multisystem atrophy, multiple sclerosis, epilepsy, bipolar disorder, schizophrenia, anxiety disorders (e.g., a panic disorder, social anxiety disorder or generalized anxiety disorder) or progressive supranuclear palsy.

It is contemplated that the compounds disclosed can be used in combination with other treatments and/or therapeutic agents. The invention embraces combination therapy, which includes the administration of a compound described herein, e.g., a compound of Formula (I) or (I)(e.g., a compound of Formula (I-a), (I-b), (I-c), (I-d), (I-e), (I-f), (I-g), (I-h), or (II-a)), or related compound described herein and a second treatment and/or agent as part of a specific treatment regimen intended to provide the beneficial effect from the co-action of these therapeutic agents.

During the treatment of Parkinson's disease, the acid ceramidase inhibitor can be administered in combination with carbidopa and/or levadopa, a dopamine agonist, a monoamine oxidase B inhibitor, a catchetol O-methyltransferase inhibitor, an anticholingeric, or amantadine. During the treatment of Alzheimer's disease, the acid ceramidase inhibitor can be administered in combination with a cholinesterase inhibitor and/or memantine. During the treatment of Huntington's disease, the acid ceramidase inhibitor can be administered in combination with tetrabenazine; an antipsychotic drug such as haloperidol, chlorpromazine, quetiapine, risperidone, and olanzapine; a chorea-suppressing medication such as amantadine, levetiracetam, and clonazempam; an antidepressant such as citalopram, fluoxetine, and sertraline; and a mood-stabilizing drug such as valproate, carbamazepine, and lamotrigine.

During the treatment of amyotrophic lateral sclerosis, the acid ceramidase inhibitor can be administered in combination with riluzole; an agent for ameliorating muscle cramps and spasms such as cyclobenzaprine HCL, metaxalone, and robaxin; an agent for ameliorating spasticity such as tizanidine HCl, baclofen, and dantrolene; an agent for ameliorating fatigue such as caffeine, caffeine citrate, or caffeine benzoate injection; an agent for ameliorating excessive salivation such as glycopyrrolate, propantheline, amitriptyline, nortriplyline HCL and scopolamine; an agent for ameliorating excessive phlegm such as guaifenesin, albuterol inhalation, and acetylcysteine; an agent for ameliorating pain such as an opioid; an anticonvulsant or antiepileptic; a serotonin reuptake inhibitor; an antidepressant; an agent for ameliorating sleep disorders such as a benzodiazepine, a non-benzodiazepine hypnotic, a melatonin receptor stimulator, an anti-narcoleptic, and an orexin receptor antagonist; and an agent pseudobulbar affect such as dextromethorphan/quinidine.

During the treatment of multiple sclerosis, the acid ceramidase inhibitor can be administered in combination with a corticosteroid, P interferon, glatiramer acetate, dimethyl fumarate, fingolimod, teriflunomide, natalizumab, mitoxantrone, baclofen, and tizanidine. During the treatment of diffuse Lewy body disease, the acid ceramidase inhibitor can be administered in combination with a cholinesterase inhibitor, a Parkinson's disease medication such as carbidopa and/or levodopa, and an anti-psychotic medication such as quetiapine and olanzapine.

During the treatment of multisystem atrophy, the acid ceramidase inhibitor can be administered in combination with a medication to raise blood pressure such as fludrocortisone, psyridostigmine, midodrine, and droxidopa; and a Parkinson's disease medication such as carbidopa and/or levodopa. During the treatment of frontotemporal dementia, the acid ceramidase inhibitor can be administered in combination with an antidepressant, a selective serotonin reuptake inhibitor, and an antipsychotic. During the treatment of progressive upranuclear palsy, the acid ceramidase inhibitor can be administered in combination with a Parkinson's disease medication such as carbidopa and/or levodopa. It is understood that other combinations would be known be those skilled in the art.

### V. Kits for Use in Medical Applications

Another aspect of the invention provides a kit for treating a disorder. The kit comprises: i) instructions for treating a medical disorder, such as, cancer (such as melanoma), a lysosomal storage disorder (such as Krabbe disease, Fabry disease, Tay-Sachs disease, Pompe disease, Hunter's syndrome, Niemann Pick disease Types A and B, Gaucher disease), a neurodegenerative disease (such as Alzheimer's disease, Parkinson's disease, Huntington's disease, and amyotrophic lateral sclerosis), an inflammatory disorder, and pain; and ii) a compound described herein or related organic compound described herein, such as a compound of Formula (I) or (II) (e.g., a compound of Formula (I-a), (I-b), (I-c), (I-d), (I-e), (I-f), (I-g), (I-h), or (II-a)), or a composition described herein. The kit may comprise one or more unit dosage forms containing an amount of a compound described herein or related organic compound described herein, such as a compound of Formula (I) or (II) (e.g., a compound of Formula (I-a), (I-b), (I-c), (I-d), (I-e), (I-f), (I-g), (I-h), or (II-a)), that is effective for treating said medical disorder, for example, cancer (such as melanoma), lysosomal storage disorder (such as Krabbe disease, Fabry disease, Tay-Sachs disease, Pompe disease, Hunter's syndrome, Niemann Pick disease Types A and B, Gaucher disease), neurodegenerative disease (such as Alzheimer's disease, Parkinson's disease, Huntington's disease, and amyotrophic lateral sclerosis), inflammatory disorder, and pain.

The description above describes multiple aspects and embodiments of the invention, including substituted benzimidazole carboxamides and related organic compounds, compositions comprising a substituted benzimidazole carboxamides or related organic compounds, methods of using the substituted benzimidazole carboxamides or related organic compounds, and kits. The patent application specifically contemplates all combinations and permutations of the aspects and embodiments. For example, the invention contemplates treating a medical disorder such as Gaucher disease, Parkinson's disease, Lewy body disease, dementia, or multiple system atrophy in a human patient by administering a therapeutically effective amount of a compound described herein, e.g., a compound of Formula (I) or (II) (e.g., a compound of Formula (I-a), (I-b), (I-c), (I-d), (I-e), (I-f), (I-g), (I-h), or (II-a)), or a composition comprising such a compound. Further, for example, the invention contemplates a kit for treating a medical disorder such as cancer (such as melanoma), lysosomal storage disorder (such as Krabbe disease, Fabry disease, Tay-Sachs disease, Pompe disease, Hunter's syndrome, Niemann Pick disease Types A and B, Gaucher disease), neurodegenerative disease (such as Alzheimer's disease, Parkinson's disease, Huntington's disease, and amyotrophic lateral sclerosis), inflammatory disorder, and pain and ii) a compound described herein or related organic compound described herein, such as a compound of Formula (I) or (II) (e.g., a compound of Formula (I-a), (I-b), (I-c), (I-d), (I-e), (I-f), (I-g), (I-h), or (II-a)), or a composition comprising such a compound.

In another aspect, the invention provides a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for use in a method of treating a subject with cancer and in need thereof, the method comprising administering to the subject a therapeutically effective amount of the compound or the pharmaceutical composition.

In another aspect, the invention provides a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for use in a method of treating a subject with a lysosomal storage disorder and in need thereof, the method comprising administering to the subject a therapeutically effective amount of the compound or the pharmaceutical composition.

In another aspect, the invention provides a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for use in a method of treating a subject with a neurodegenerative disorder and in need thereof, the method comprising administering to the subject a therapeutically effective amount of the compound or the pharmaceutical composition.

In another aspect, the invention provides a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for use in a method of treating a subject with an inflammatory disorder and in need thereof, the method comprising administering to the subject a therapeutically effective amount of the compound or the pharmaceutical composition.

In another aspect, the invention provides use of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for the manufacture of a medicament for treating a subject with cancer and in need thereof, the method comprising administering to the subject a therapeutically effective amount of the compound or the pharmaceutical composition.

In another aspect, the invention provides use of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for the manufacture of a medicament for treating a subject with a lysosomal storage disorder and in need thereof, the method comprising administering to the subject a therapeutically effective amount of the compound or the pharmaceutical composition.

In another aspect, the invention provides use of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for the manufacture of a medicament for treating a subject with a neurodegenerative disorder and in need thereof, the method comprising administering to the subject a therapeutically effective amount of the compound or the pharmaceutical composition.

In another aspect, the invention provides use of a compound (e.g., a compound of Formula (I) or (II) (e.g., a compound of Formula (I-a), (I-b), (I-c), (I-d), (I-e), (I-f), (I-g), (I-h), or (II-a)) or a pharmaceutical composition as disclosed herein for the manufacture of a medicament for treating a subject with an inflammatory disorder and in need thereof, the method comprising administering to the subject a therapeutically effective amount of the compound or the pharmaceutical composition.

### EXAMPLES

The invention now being generally described, will be more readily understood by reference to the following examples, which are included merely for purposes of illustration of certain aspects and embodiments of the present invention, and are not intended to limit the invention. In certain instances, the amount of compound produced by the procedure is stated along with the yield, which may be presented in the format of the procedure produced the title compound (10 mg; 90%) which means that 10 mg of the title compound was obtained and that corresponds to a yield of 90%.

### Example A—Preparation of Benzimidazole Carboxamide Compounds

Benzimidazole carboxamide compounds were prepared based on general procedures described in Part I below. Specific benzimidazole carboxamide compounds prepared according to the general procedures are provided in Part II below.

### Part I—General Procedures

### General Procedure A for the Preparation of Coupled Aryl and Heteroaryl Groups Using Suzuki Catalyzed Coupling Conditions Between an Organoboronic Acid or Ester and an Heteroaryl Halide

A suspension of heteroaryl halide (1.0 eq), organoboronic acid or organoboronic ester (1.2 eq), K₃PO₄ (3.0 eq) or Na₂CO₃ (3.0 eq), and Pd(dppf)Cl₂.DCM (5 mol %) or Pd₂(dba)₃ (10 mol %) in DME or 1,4-dioxane (40 mL/mmol) was stirred at 70-100° C. for 2-24 h under N₂. Then, the reaction mixture was quenched with water (30 mL/mmol) and the resulting mixture was extracted with EtOAc (30 mL/mmol×3). The combined organic layers were washed with water (30 mL/mmol) and brine (30 mL/mmol), dried over anhydrous Na₂SO₄, and filtered. The filtrate was concentrated in vacuo, and the resulting residue was purified by silica gel column chromatography to afford the coupled ring system.

### General Procedure B for the Preparation of Benzimidazole Carboxamides

To a solution of a substituted benzimidazole (1.0 eq) and Et₃N (2.0-5.0 eq) in DCM or CH₃CN (5-20 mL/mmol) was added isocyanate (e.g., (2-isocyanatoethyl)benzene) (1.2-4.0 eq) at 0° C. or at RT. The resulting mixture was stirred at RT or at reflux for 2 h to overnight. The reaction mixture was poured into water and extracted with DCM. The combined organic layers were washed with brine, dried over Na₂SO₄, and filtered. The filtrate was concentrated and purified by silica gel column chromatography or Prep-HPLC to give a benzimidazole carboxamide, which was further triturated with organic solvents if needed to increase the purity.

General procedure C for the preparation of benzimidazole carboxamides

To a solution of a substituted benzimidazole (1.0 eq) and Et₃N (2.0-5.0 eq) in DCM (5-20 mL/mmol) was added a solution of 4-nitrophenyl chloroformate (1.2-2.0 eq) in DCM (2-10 mL/mmol) at 0° C. The mixture was stirred for 10-30 min at 0° C. and then the amine (1.5-5.0 eq) was added. The resulting mixture was stirred for 30-60 min at 0° C. The reaction mixture was poured into water and extracted with DCM. The combined organic layers were washed with brine, dried over Na₂SO₄, filtered. The filtrate was concentrated and purified by silica gel column chromatography to give a benzimidazole carboxamide, which was further triturated with organic solvents if needed to increase the purity.

### General Procedure D for the Preparation of Benzimidazole Carboxamides

To a solution of a substituted benzimidazole (1.0 eq) and Et₃N (2.0 eq) in DCM (3-10 mL/mmol) was added a solution of 2,4-dinitrophenyl carbonochloridate (1.5 eq) in DCM (3-10 mL/mmol) at −78° C. The mixture was stirred at −78° C. for 30 min and then the amine (2.0 eq) was added. The resulting mixture was stirred at −78° C. for another 30 min. The reaction mixture was purified directly by silica gel column chromatography without any further work-up for instability to give a benzimidazole carboxamide.

### General Procedure E for the Preparation of Amino Substituted Nitroaniline

To a suspension of a substituted nitroaniline (1.0 eq)) and K₂CO₃ (2.0 eq) in CH₃CN (1.0 M) was added an amine (1.5 eq) at RT and the resulting mixture was stirred at 70-80° C. for 2-15 h. The reaction mixture was concentrated to give a residue which was purified by silica gel column chromatography (MeOH:DCM; 1:6 to 1:5) affording the amino substituted nitroaniline.

### General Procedure F for the Preparation of Aromatic Diamines

To a solution of the amino substituted nitroaniline (1.0 eq) in MeOH (0.3-0.5M) was added Pd/C (25 wt. %) at RT and the mixture was stirred at RT under an atmospheric pressure of H₂ for 4 h. The mixture was filtered and concentrated to give a residue which was used directly or purified by silica gel column chromatography to afford the aromatic diamine.

### General Procedure G for the Preparation of Substituted Benzimidazoles

Method A: To a solution of aromatic diamine (1.0 eq) in AcOH (0.6 M) was added tetramethoxymethane (2.5 eq) at RT and the resulting mixture was stirred at RT for 1-3 h or at 60-100° C. for 1-5 h. The reaction mixture was concentrated and partitioned between saturated aq. NaHCO₃ solution and EtOAc. The organic layer was dried over Na₂SO₄, filtered and concentrated to give a residue that was either used directly or purified by silica gel column chromatography (MeOH:DCM; 1:5 to 1:20) to afford the substituted benzimidazole.

Method B: To a solution of aromatic diamine (1.0 eq) in formic acid (0.6 M) was heated at 120° C. for 1 h then cooled to RT. The pH value was adjusted to 9 with saturated aq. NaHCO₃ solution and the mixture was extracted with DCM (×3). The combined organic layers were dried over anhydrous Na₂SO₄, filtered, and concentrated to give the substituted benzimidazole which was used directly in the next step.

### General Procedure H for the Preparation of Heteroaryl Cycloalkyl Amines Using Buchwald Catalyzed Coupling Conditions Between a Cycloalkyl Amine and an Heteroaryl Halide

A mixture of heteroaryl halide (1.0 eq), cycloalkyl amine (3.0 eq), Pd₂(dba)₃ (5 mol %), BINAP or XPhos (10 mol %), t-BuOK (2.0 eq) and toluene (0.2-0.5 M) was stirred at 90-100° C. for 12-16 h. The solid was filtered off and the solution was concentrated to give a residue, which was purified by silica gel column chromatography (DCM:MeOH=10:1) to give the heteroaryl cycloalkyl amine.

### Part II—Preparation of Specific Benzimidazole Compounds

Exemplary procedures for preparing specific benzimidazole carboxamides are provided below. Some examples describe the multistep synthesis of benzimidazole carboxamides where the various steps, including the synthesis of intermediates, are discussed in detail.

### Example 1—N-Phenethyl-1H-benzo[d]imidazole-1-carboxamide

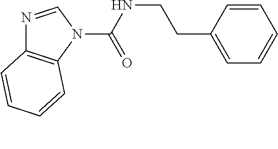

Following general procedure B, 1H-benzo[d]imidazole (0.20 g, 1.69 mmol) and (2-isocyanatoethyl)benzene (0.37 g, 2.54 mmol) afforded the title compound (35.0 mg, 8%) as a white solid. ¹H NMR (400 MHz, DMSO-d₆) δ 8.68-8.66 (m, 2H), 8.02 (d, J=7.2 Hz, 1H), 7.70 (d, J=7.2 Hz, 1H), 7.36-7.22 (m, 7H), 3.57-3.52 (m, 2H), 2.91 (t, J=7.6 Hz, 2H). LC-MS m/z: 266.2 [M+H]⁺. HPLC Purity (254 nm): 100%; t_{R}=1.89 min.

### Example 2—2-Methyl-N-phenethyl-1H-benzo[d]imidazole-1-carboxamide

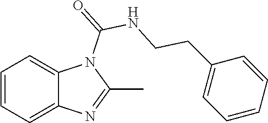 [SMILES: Cc1nc2ccccc2n1C(=O)NCCc1ccccc1]

Following procedure B, 2-methyl-1H-benzo[d]imidazole (200 mg, 1.52 mmol) and (2-isocyanatoethyl)benzene (445 mg, 3.03 mmol) afforded the title compound (23.1 mg, 5.5%) as a white solid. ¹H NMR (500 MHz, CDCl₃) δ 7.65 (d, J=7.9 Hz, 1H), 7.41-7.34 (m, 2H), 7.30 (dd, J=8.7, 7.1 Hz, 3H), 7.24 (d, J=7.2 Hz, 1H), 7.14 (t, J=7.4 Hz, 1H), 7.06 (d, J=8.1 Hz, 1H), 5.73 (s, 1H), 3.83 (dt, J=12.6, 6.6 Hz, 2H), 3.05 (t, J=6.8 Hz, 2H), 2.71 (s, 3H). LC-MS m/z: 280.1 [M+H]⁺. HPLC Purity (254 nm): >99%; t_{R}=7.08 min.

### Example 3—2-Methoxy-N-phenethyl-1H-benzo[d]imidazole-1-carboxamide

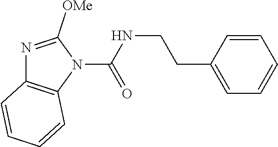 [SMILES: COc1nc2ccccc2n1C(=O)NCCc1ccccc1]

Following general procedure G (method A), benzene-1,2-diamine (2.0 g, 18.5 mmol) afforded 2-methoxy-1H-benzo[d]imidazole (2.1 g, 77%) as a white solid. LC-MS m/z: 149.0 [M+H]⁺. HPLC Purity (214 nm): 100%; t_{R}=1.57 min.

Following general procedure B, 2-methoxy-1H-benzo[d]imidazole (200 mg, 1.35 mmol) and (2-isocyanatoethyl)benzene (238 mg, 1.62 mmol) afforded the title compound (157.9 mg, 40%) as a white solid. ¹H NMR (400 MHz, CDCl₃) δ 8.18-8.15 (m, 1H), 7.50-7.47 (m, 1H), 7.37 (t, J=7.2 Hz, 2H), 7.31-7.27 (m, 3H), 7.26-7.21 (m, 2H), 6.96 (s, 1H), 4.14 (s, 3H), 3.74 (q, J=5.6 Hz, 2H), 2.96 (t, J=6.6 Hz, 2H). LC-MS m/z: 296.3 [M+H]⁺. HPLC Purity (214 nm): 100%; t_{R}=6.55 min.

### Example 4—5-(1-Methylpiperidin-4-yl)-N-phenethyl-1H-bezo[d]imidazole-1-carboxamide

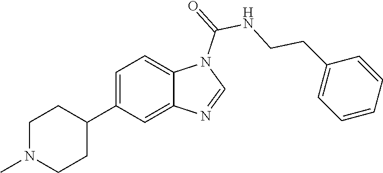 [SMILES: CN1CCC(c2ccc3c(c2)ncn3C(=O)NCCc2ccccc2)CC1]

Using general procedure A, 5-bromo-1H-benzo[d]imidazole (400 mg, 2.0 mmol) and 1-methyl-4-(4,4,5,5-tetramethyl-1,3,2-dioxaborolan-2-yl)-1,2,3,6-tetrahydropyridine (579 mg, 2.6 mmol), afforded crude 5-(1-methyl-1,2,3,6-tetrahydropyridin-4-yl)-1H-benzo[d]imidazole (430 mg) as a yellow solid. LC-MS m/z: 214.0 [M+H]⁺. Purity (214 nm): 91%; t_{R}=1.48 min.

A solution of 5-(1-methyl-1,2,3,6-tetrahydropyridin-4-yl)-1H-benzo[d]imidazole (426 mg, 2 mmol), Pd(OH)₂ (50 mg, 0.2 mmol) in MeOH (10 mL) was stirred at 50° C. for 5 h and filtered. The filtrate was concentrated to afford 5-(1-methylpiperidin-4-yl)-1H-benzo[d]imidazole (410 mg, 95%) as yellow solid. LC-MS m/z: 216.0 [M+H]⁺. HPLC Purity (214 nm): 99%; t_{R}=1.44 min.

Following general procedure B, 5-(1-methylpiperidin-4-yl)-1H-benzo[d]imidazole (215 mg, 1.0 mmol) and (2-isocyanatoethyl)benzene (162 mg, 1.1 mmol) afforded the title compound (54.9 mg, 15%) as a white solid. ¹H NMR (400 MHz, CDCl₃) δ 8.33 (d, J=13.4 Hz, 1H), 7.65 (d, J=1.4 Hz, 1H), 7.43-7.26 (m, 6H), 7.20 (dd, J=8.5, 1.6 Hz, 1H), 5.75 (s, 1H), 3.81 (dd, J=12.5, 6.7 Hz, 2H), 3.03 (dd, J=15.5, 8.9 Hz, 4H), 2.69-2.57 (m, 1H), 2.47-2.29 (m, 3H), 2.13 (d, J=11.3 Hz, 2H), 1.90 (dd, J=14.6, 8.8 Hz, 4H). LC-MS m/z: 363.1 [M+H]⁺. HPLC Purity (214 nm): 96.02%; t_{R}=6.25 min.

### Example 5—N-(3-Phenylpropyl)-1H-benzo[d]imidazole-1-carboxamide

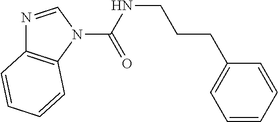 [SMILES: O=C(NCCCc1ccccc1)n1cnc2ccccc21]

Following general procedure D, 1H-benzo[d]imidazole (0.30 g, 2.54 mmol) and 3-phenylpropyl amine (0.38 g, 2.80 mmol) gave the title compound (101.5 mg, 14%) as a white solid. ¹H NMR (400 MHz, CDCl₃) δ 8.16 (s, 1H), 7.81-7.72 (m, 2H), 7.40-7.20 (m, 7H), 5.65 (br s, 1H), 3.58 (dd, J=12.8, 6.8 Hz, 2H), 2.79 (t, J=7.6 Hz, 2H), 2.11-2.04 (m, 2H). LC-MS m/z: 280.2 [M+H]⁺. HPLC Purity (254 nm): 100%; t_{R}=1.96 min.

### Example 6-N-(4-Phenylbutyl)-1H-benzo[d]imidazole-1-carboxamide

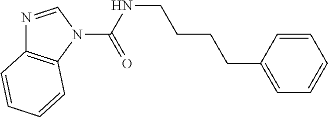 [SMILES: O=C(NCCCCc1ccccc1)n1cnc2ccccc21]

Following general procedure D, 1H-benzo[d]imidazole (0.50 g, 4.24 mmol) and 4-phenylbutylamine (0.69 g, 4.66 mmol) gave the title compound (57.8 mg, 5%) as a white solid. ¹H NMR (400 MHz, CDCl₃) δ 8.38 (s, 1H), 7.84-7.75 (m, 2H), 7.42-7.39 (m, 2H), 7.38-7.18 (m, 5H), 5.68 (br s, 1H), 3.55 (dd, J=12.8, 6.8 Hz, 2H), 2.70 (t, J/=7.2 Hz, 2H), 1.75 (m, 4H). LC-MS m/z: 294.2 [M+H]⁺. HPLC Purity (254 nm): 100%; t_{R}=2.02 min.

### Examples 7a and 7b—6-Chloro-N-phenethyl-5-(pyridin-2-yl)-1H-benzo[d]imidazole-1-carboxamide and 5-chloro-N-phenethyl-6-(pyridin-2-yl)-1H-benzo[d]imidazole-1-carboxamide

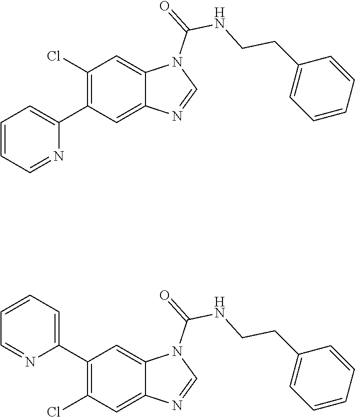 [SMILES: O=C(NCCc1ccccc1)n1cnc2cc(-c3ccccn3)c(Cl)cc21 ; O=C(NCCc1ccccc1)n1cnc2cc(Cl)c(-c3ccccn3)cc21]

A mixture of 4-bromo-5-chloro-2-nitroaniline (2.0 g, 7.79 mmol) and SnCl₂.2H₂O (8.96 g, 39.8 mmol) in 20 mL of EtOH was heated at reflux for 3 h. The reaction mixture was concentrated in vacuo, dissolved in water and solid NaHCO₃ was added to adjust the pH to 8. The mixture was filtered, and the filter cake was triturated with DCM/MeOH (50 mL). The filtrate was concentrated to give 4-bromo-5-chlorobenzene-1,2-diamine (1.5 g, 85%) as a white solid. LC-MS m/z: 222.9 [M+H]⁺. HPLC Purity (214 nm): 98%; t_{R}=1.75 min.

Following general procedure G (method B), 4-bromo-5-chlorobenzene-1,2-diamine (1.5 g, 6.78 mmol) afforded 6-bromo-5-chloro-1H-benzo[d]imidazole (1.4 g, 90%) as a white solid. LC-MS m/z: 232.9 [M+H]⁺. HPLC Purity (214 nm): 95%; t_{R}=1.61 min.

A solution of 6-bromo-5-chloro-1H-benzo[d]imidazole (1.0 g, 4.32 mmol), 2-(tributylstannyl)pyridine (2.39 g, 1.5 mmol), Pd(PPh₃)₄ (499 mg, 0.43 mmol) and CuBr (62 mg, 0.43 mmol) in dioxane/DMF (10 mL) was stirred at 100° C. for 4 h under microwave conditions. The reaction was concentrated and purified by prep-chromatography (MeCN/10 mM NH₄HCO₃) to afford 5-chloro-6-(pyridin-2-yl)-1H-benzo[d]imidazole (330 mg, 33%) as a white solid. LC-MS m/z: 229.9 [M+H]⁺. HPLC Purity (254 nm): 96%; t_{R}=1.67 min.

Following general procedure B, 5-chloro-6-(pyridin-2-yl)-1H-benzo[d]imidazole (150 mg, 0.66 mmol) and (2-isocyanatoethyl)benzene (144 mg, 0.98 mmol) afforded 6-chloro-N-phenethyl-5-(pyridin-2-yl)-1H-benzo[d]imidazole-1-carboxamide (38.4 mg, 15%) and 5-chloro-N-phenethyl-6-(pyridin-2-yl)-1H-benzo[d]imidazole-1-carboxamide (79.4 mg, 32%) as white solids.

6-chloro-N-phenethyl-5-(pyridin-2-yl)-1H-benzo[d]imidazole-1-carboxamide: ¹H NMR (400 MHz, CDCl₃) δ 8.44 (d, J=4.4 Hz, 1H), 8.14 (s, 1H), 7.83-7.78 (m, 2H), 7.75 (s, 1H), 7.54 (d, J=7.6 Hz, 1H), 7.41-7.37 (m, 2H), 7.33-7.28 (m, 4H), 7.00-6.99 (m, 1H), 3.77 (q, J=6.4 Hz, 2H), 3.02 (t, J=7.2 Hz, 2H). LC-MS m/z: 376.9 [M+H]⁺. HPLC Purity (214 nm): 97%, t_{R}=7.47 min.

5-chloro-N-phenethyl-6-(pyridin-2-yl)-1H-benzo[d]imidazole-1-carboxamide: ¹H NMR (400 MHz, CDCl₃) δ 8.62 (d, J=4.4 Hz, 1H), 8.33 (s, 1H), 7.87 (s, 1H), 7.83 (td, J=8.0 Hz, 2.0 Hz, 1H), 7.69-7.66 (m, 2H), 7.38-7.35 (m, 1H), 7.24-7.17 (m, 4H), 7.12-7.08 (m, 1H), 6.68-6.66 (m, 1H), 3.77 (q, J=6.4 Hz, 2H), 3.00 (t, J=6.8 Hz, 2H). LC-MS m/z: 377.0 [M+H]⁺. HPLC Purity (214 nm): 98%; t_{R}=7.53 min.

### Examples 8a and 8b—N-Phenethyl-5-(pyrimidin-5-yl)-1H-benzo[d]imidazole-1-carboxamide and N-phenethyl-6-(pyrimidin-5-yl)-1H-benzo[d]imidazole-1-carboxamide

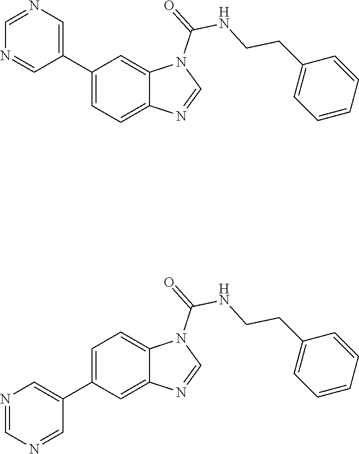 [SMILES: O=C(NCCc1ccccc1)n1cnc2ccc(-c3cncnc3)cc21 ; O=C(NCCc1ccccc1)n1cnc2cc(-c3cncnc3)ccc21]

Using general procedure A, 5-bromo-1H-benzo[d]imidazole (500 mg, 2.55 mmol) and 5-(4,4,5,5-tetramethyl-1,3,2-dioxaborolan-2-yl)pyrimidine (578 mg, 2.81 mmol) afforded 5-(pyrimidin-5-yl)-1H-benzo[d]imidazole (130 mg, 26.0%) as a brown solid. LC-MS m/z: 197.0 [M+H]⁺. HPLC Purity (254 nm): >99%; t_{R}=1.43 min.

Following general procedure B, 5-(pyrimidin-5-yl)-1H-benzo[d]imidazole (130 mg, 0.66 mmol) and (2-isocyanatoethyl)benzene (195 mg, 1.33 mmol) gave N-phenethyl-5-(pyrimidin-5-yl)-1H-benzo[d]imidazole-1-carboxamide (7.2 mg 3.2%) and N-phenethyl-6-(pyrimidin-5-yl)-1H-benzo[d]imidazole-1-carboxamide (21.6 mg 9.5%) as white solids.

N-phenethyl-5-(pyrimidin-5-yl)-1H-benzo[d]imidazole-1-carboxamide: ¹H NMR (400 MHz, CDCl₃) δ 9.22 (s, 1H), 8.99 (s, 2H), 8.36 (s, 1H), 8.00 (d, J=1.3 Hz, 1H), 7.73 (d, J=8.5 Hz, 1H), 7.53 (dd, J=8.5, 1.6 Hz, 1H), 7.39 (t, J=7.2 Hz, 2H), 7.31 (dd, J=10.5, 9.2 Hz, 3H), 5.79 (s, 1H), 3.84 (dd, J=12.6, 6.5 Hz, 2H), 3.06 (t, J=6.7 Hz, 2H). LC-MS m/z: 344.2 [M+H]⁺. HPLC Purity (254 nm): >99%; t_{R}=7.98 min.

N-phenethyl-6-(pyrimidin-5-yl)-1H-benzo[d]imidazole-1-carboxamide: ¹H NMR (500 MHz, CDCl₃) δ 9.25 (s, 1H), 8.92 (s, 2H), 8.32 (s, 1H), 7.93 (d, J=8.4 Hz, 1H), 7.86 (d, J=1.2 Hz, 1H), 7.54 (dd, J=8.4, 1.6 Hz, 1H), 7.36-7.30 (m, 2H), 7.28 (s, 1H), 7.25-7.21 (m, 1H), 5.77 (s, 1H), 3.85 (dd, J=12.6, 6.3 Hz, 2H), 3.04 (t, J=6.6 Hz, 2H). LC-MS m/z: 344.2 [M+H]⁺. HPLC Purity (254 nm): >99%; t_{R}=7.89 min.

### Example 9—N-Phenethyl-5-(pyrimidin-2-yl)-1H-benzo[d]imidazole-1-carboxamide

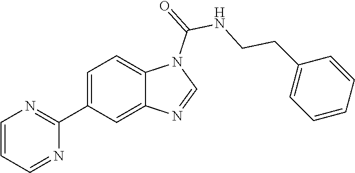 [SMILES: O=C(NCCc1ccccc1)n1cnc2cc(-c3ncccn3)ccc21]

Following general procedure B, 5-(pyrimidin-2-yl)-1H-benzo[d]imidazole (120 mg, 0.61 mmol) and (2-isocyanatoethyl)benzene (180 mg, 1.22 mmol) afforded the title compound (15.0 mg, 7.1%) as a white solid. ¹H NMR (400 MHz, CDCl₃) δ 8.91 (s, 1H), 8.83 (d, J=4.8 Hz, 2H), 8.46 (dd, J=8.7, 1.4 Hz, 1H), 8.39 (s, 1H), 7.56 (d, J=8.6 Hz, 1H), 7.39 (t, J=7.2 Hz, 2H), 7.31 (dd, J=12.6, 7.0 Hz, 3H), 7.21 (t, J=4.8 Hz, 1H), 5.80 (s, 1H), 3.84 (dd, J=12.6, 6.5 Hz, 2H), 3.05 (t, J=6.7 Hz, 2H). LC-MS m/z: 344.3 [M+H]⁺. HPLC Purity (214 nm): >99%; t_{R}=1.20 min.

### Examples 10a and 10b—N-Phenethyl-5-(pyridin-3-yl)-1H-benzo[d]imidazole-1-carboxamide and N-phenethyl-6-(pyridin-3-yl)-1H-benzo[d]imidazole-1-carboxamide

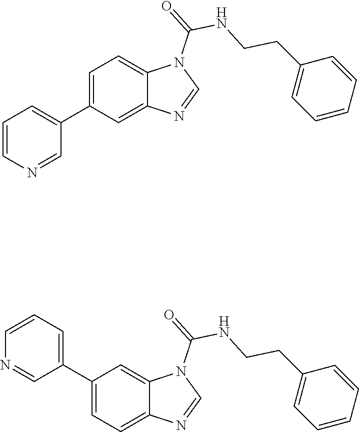 [SMILES: O=C(NCCc1ccccc1)n1cnc2cc(-c3cccnc3)ccc21 ; O=C(NCCc1ccccc1)n1cnc2ccc(-c3cccnc3)cc21]

Following general procedure A, 5-bromo-1H-benzo[d]imidazole (196 mg, 1.0 mmol) and pyridin-3-ylboronic acid (246 mg, 2.0 mmol) afforded 5-(pyridin-3-yl)-1H-benzo[d]imidazole (200 mg, 93%) as a yellow solid. LC-MS m/z: 196.0 [M+H]⁺. HPLC Purity (214 nm): 93%; t_{R}=1.55 min.

Following general procedure B, 5-(pyridin-3-yl)-1H-benzo[d]imidazole (196 mg, 1.0 mmol) and (2-isocyanatoethyl)benzene (220 mg, 1.5 mmol) gave N-phenethyl-5-(pyridin-3-yl)-1H-benzo[d]imidazole-1-carboxamide (89 mg, 22%) and N-phenethyl-6-(pyridin-3-yl)-1H-benzo[d]imidazole-1-carboxamide (110 mg, 32%) as white solids.

N-phenethyl-5-(pyridin-3-yl)-1H-benzo[d]imidazole-1-carboxamide: ¹H NMR (400 MHz, CDCl₃) δ 8.88 (d, J=1.7 Hz, 1H), 8.60 (dd, J=4.8, 1.6 Hz, 1H), 8.35 (d, J=10.9 Hz, 1H), 7.99 (d, J=1.3 Hz, 1H), 7.91 (ddd, J=15.3, 8.5, 6.5 Hz, 1H), 7.63 (d, J=8.3 Hz, 1H), 7.53 (dd, J=8.5, 1.7 Hz, 1H), 7.46-7.34 (m, 3H), 7.35-7.27 (m, 3H), 5.81 (s, 1H), 3.84 (dd, J=12.5, 6.7 Hz, 2H), 3.05 (t, J=6.7 Hz, 2H). LC-MS m/z: 343.0 [M+H]⁺. HPLC Purity (214 nm): 98%; t_{R}=6.33 min.

N-phenethyl-6-(pyridin-3-yl)-1H-benzo[d]imidazole-1-carboxamide: ¹H NMR (400 MHz, CDCl₃) δ 8.79 (d, J=2.3 Hz, 1H), 8.70-8.56 (m, 1H), 8.34 (d, J=9.6 Hz, 1H), 8.05-7.75 (m, 3H), 7.69-7.47 (m, 1H), 7.39 (dd, J=7.9, 4.8 Hz, 1H), 7.29 (dd, J=14.4, 7.3 Hz, 3H), 7.26-7.15 (m, 2H), 6.02 (s, 1H), 3.83 (dd, J=12.6, 6.5 Hz, 2H), 3.04 (t, J=6.7 Hz, 2H). LC-MS m/z: 343.1 [M+H]⁺. HPLC Purity (214 nm): 98%; t_{R}=5.95 min.

### Examples 11a and 11b —5-Chloro-6-(1-methylpiperidin-4-yl)-N-phenethyl-1H-benzo[d]imidazole-1-carboxamide and 6-Chloro-5-(1-methylpiperidin-4-yl)-N-phenethyl-1H-benzo[d]imidazole-1-carboxamide

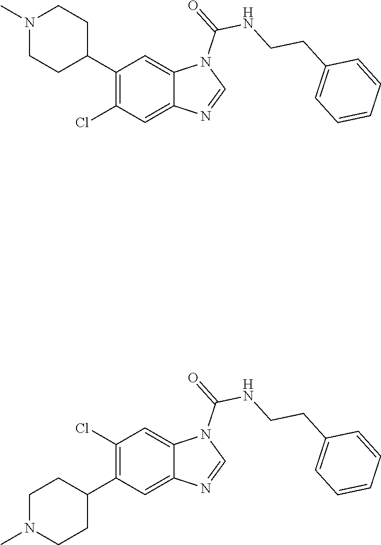 [SMILES: CN1CCC(c2cc3c(cc2Cl)ncn3C(=O)NCCc2ccccc2)CC1 ; CN1CCC(c2cc3ncn(C(=O)NCCc4ccccc4)c3cc2Cl)CC1]

Following general procedure A, 4-bromo-5-chloro-2-nitroaniline (1.5 g, 5.97 mmol) and 1-methyl-4-(4,4,5,5-tetramethyl-1,3,2-dioxaborolan-2-yl)-1,2,3,6-tetrahydropyridine (1.47 g, 6.57 mmol), afforded 5-chloro-4-(1-methyl-1,2,3,6-tetrahydropyridin-4-yl)-2-nitroaniline (1.0 g, 62%) as a brown solid. LC-MS m/z: 267.9 [M+H]⁺. HPLC Purity (214 nm): 93%; t_{R}=1.86 min.

To a solution of 5-chloro-4-(1-methyl-1,2,3,6-tetrahydropyridin-4-yl)-2-nitroaniline (1.0 g, 3.74 mmol) in MeOH (100.0 mL) was added PtO₂ (300 mg). The mixture was stirred at RT under H₂ for 16 h. The mixture was filtered, concentrated and purified by prep-chromatography (MeCN/10 mM NH₄HCO₃) to afford 4-chloro-5-(1-methylpiperidin-4-yl)benzene-1,2-diamine (450 mg, 50%) as a yellow solid. LC-MS m/z: 240.0 [M+H]⁺. HPLC Purity (214 nm): 90%; t_{R}=2.18 min.

Following general procedure G (method B), 4-chloro-5-(1-methylpiperidin-4-yl)benzene-1,2-diamine (450 mg, 1.88 mmol) afforded 5-chloro-6-(1-methylpiperidin-4-yl)-1H-benzo[d]imidazole (350 mg, 75%) as a white solid. LC-MS m/z: 250.1 [M+H]⁺. HPLC Purity (214 nm): 90%; t_{R}=1.67 min.

Following general procedure B, 5-chloro-6-(1-methylpiperidin-4-yl)-1H-benzo[d]imidazole (120 mg, 0.48 mmol) and (2-isocyanatoethyl)benzene (106 mg, 0.72 mmol) afforded 5-chloro-6-(1-methylpiperidin-4-yl)-N-phenethyl-1H-benzo[d]imidazole-1-carboxamide (10.5 mg, 5%) and 6-chloro-5-(1-methylpiperidin-4-yl)-N-phenethyl-1H-benzo[d]imidazole-1-carboxamide (37.6 mg, 19%) as white solids.

5-chloro-6-(1-methylpiperidin-4-yl)-N-phenethyl-1H-benzo[d]imidazole-1-carboxamide: ¹H NMR (400 MHz, CDCl₃) δ 8.54 (s, 1H), 8.06 (s, 1H), 7.78 (s, 1H), 7.33-7.21 (m, 5H), 3.80 (q, J=6.8 Hz, 2H), 3.45 (d, J=11.6 Hz, 2H), 3.37-3.33 (m, 1H), 3.05 (t, J=7.6 Hz, 2H), 2.74-2.69 (m, 5H), 2.54-2.45 (m, 2H), 1.97-1.94 (m, 2H). LC-MS m/z: 397.0 [M+H]⁺. HPLC Purity (254 nm): 96%; t_{R}=5.33 min.

6-chloro-5-(1-methylpiperidin-4-yl)-N-phenethyl-1H-benzo[d]imidazole-1-carboxamide: ¹H NMR (400 MHz, DMSO-d₆) δ 8.78 (t, J=5.2 Hz, 1H), 8.71 (s, 1H), 8.02 (s, 1H), 7.68 (s, 1H), 7.34-7.22 (m, 5H), 3.53 (q, J=6.0 Hz, 2H), 2.97-2.89 (m, 5H), 2.26 (s, 3H), 2.11 (t, J=6.4 Hz, 2H), 1.78-1.70 (m, 4H). LC-MS m/z: 397.0 [M+H]⁺. HPLC Purity (214 nm): 99% N; t_{R}=5.81 min.

### Example 12—4-(1-Methylpiperidin-4-yl)-N-phenethyl-1H-benzo[d]imidazole-1-carboxamide

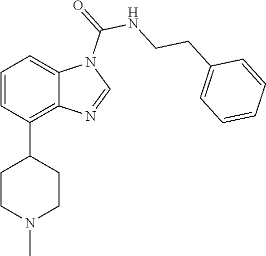 [SMILES: CN1CCC(c2cccc3c2ncn3C(=O)NCCc2ccccc2)CC1]

Following general procedure A, 4-bromo-1H-benzo[d]imidazole (600 mg, 3.1 mmol) and 1-methyl-4-(4,4,5,5-tetramethyl-1,3,2-dioxaborolan-2-yl)-1,2,3,6-tetrahydropyridine (1.02 g, 4.6 mmol) afforded 4-(1-methyl-1,2,3,6-tetrahydropyridin-4-yl)-1H-benzo[d]imidazole (600 mg, 30%) as a yellow solid. LC-MS m/z: 214.0 [M+H]⁺. HPLC Purity (254 nm): >98%; t_{R}=1.59 min.

A mixture of 4-(1-methyl-1,2,3,6-tetrahydropyridin-4-yl)-1H-benzo[d]imidazole (600 mg, 2.8 mmol) and PtO₂ (100 mg) in MeOH (30 mL) was stirred at 30° C. under H₂ for 6 days. The reaction was filtered, washed with MeOH (10 mL), concentrated, and the residue was purified by silica gel column chromatography (DCM:MeOH=4:1) to give 4-(1-methylpiperidin-4-yl)-1H-benzo[d]imidazole (420 mg, 69%) as a yellow oil. LC-MS m/z: 216.1 [M+H]⁺. HPLC Purity (254 nm): >89%; t_{R}=1.42 min.

Following general procedure B, 4-(1-methylpiperidin-4-yl)-1H-benzo[d]imidazole (100 mg, 0.47 mmol) and (2-isocyanatoethyl)benzene (137 mg, 0.93 mmol) afforded the title compound (29.0 mg, 17.1%) as a yellow solid. ¹H NMR (400 MHz, CDCl₃) δ 8.40 (s, 1H), 7.36 (t, J=7.0 Hz, 3H), 7.32-7.23 (m, 4H), 7.20 (d, J=7.4 Hz, 1H), 6.21 (s, 1H), 3.80 (dd, J=12.6, 6.8 Hz, 2H), 3.58-3.37 (m, 1H), 3.31 (d, J=11.5 Hz, 2H), 3.06-2.97 (m, 2H), 2.80-2.71 (m, 1H), 2.50 (m, 4H), 2.18 (dd, J=22.8, 12.2 Hz, 2H), 2.03 (d, J=13.2 Hz, 2H). LC-MS m/z: 363.0 [M+H]⁺. HPLC Purity (214 nm): >99%; t_{R}=1.59 min.

### Examples 13a and 13b—N-Phenethyl-5-(piperidin-1-yl)-1H-benzo[d]imidazole-1-carboxamide and N-Phenethyl-6-(piperidin-1-yl)-1H-benzo[d]imidazole-1-carboxamide

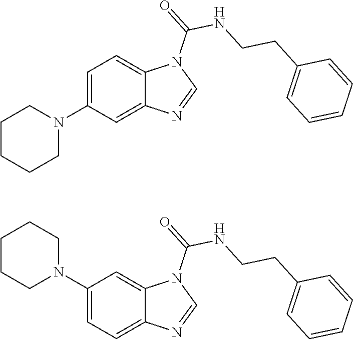 [SMILES: O=C(NCCc1ccccc1)n1cnc2cc(N3CCCCC3)ccc21 ; O=C(NCCc1ccccc1)n1cnc2ccc(N3CCCCC3)cc21]

A mixture of 5-bromo-1H-benzo[d]imidazole (800 mg, 4.06 mmol), NaH (214 mg, 8.93 mmol) and THF (20 mL) was stirred at 0° C. for 1 h followed by the addition of 1-chloromethyl-4-methoxybenzene (950 mg, 6.09 mmol) and the mixture was heated at 60° C. for 16 h. Then the mixture was poured into water (20 mL) and extracted with EA (100 mL×3). The combined organic layers were dried over Na₂SO₄, filtered and concentrated to give a residue which was purified by silica gel column chromatography (DCM:MeOH=15:1) to give 5-bromo-1-(4-methoxybenzyl)-1H-benzo[d]imidazole (990 mg, 77.2%). LC-MS m/z: 316.8 [M+H]⁺. HPLC Purity (214 nm): >97%; t_{R}=1.98 min.

Following general procedure H, 5-bromo-1-(4-methoxybenzyl)-1H-benzo[d]imidazole (990 mg, 3.13 mmol) and piperidine (807 mg, 9.49 mmol) afforded 1-(4-methoxybenzyl)-5-(piperidin-1-yl)-1H-benzo[d]imidazole (200 mg, 19.9%) as a brown solid. LC-MS m/z: 322.0 [M+H]⁺. HPLC Purity (254 nm): >68%; t_{R}=2.29 min.

A mixture of 1-(4-methoxybenzyl)-5-(piperidin-1-yl)-1H-benzo[d]imidazole (200 mg, 0.62 mmol) and TFA (1 mL) was stirred at 130° C. for 2 h under microwave conditions. Then Na₂CO₃ solution (5 mL) was added and the solution was extracted with DCM (100 mL×3). The solution was concentrated and purified by silica gel column chromatography (DCM:MeOH=20:1) to give 5-(piperidin-1-yl)-1H-benzo[d]imidazole (120 mg, 96.0%) as a brown solid. LC-MS m/z: 202.0 [M+H]⁺. HPLC Purity (254 nm): >87%; t_{R}=2.28 min.

Following general procedure B, 5-(piperidin-1-yl)-1H-benzo[d]imidazole (70 mg, 0.35 mmol), and (2-isocyanatoethyl)benzene (102 mg, 0.69 mmol) gave N-phenethyl-5-(piperidin-1-yl)-1H-benzo[d]imidazole-1-carboxamide (13.3 mg, 11.0%) and N-phenethyl-6-(piperidin-1-yl)-1H-benzo[d]imidazole-1-carboxamide (12.1 mg, 10.0%) as white solids.

N-phenethyl-5-(piperidin-1-yl)-1H-benzo[d]imidazole-1-carboxamide: ¹H NMR (500 MHz, CDCl₃) δ 8.29 (s, 1H), 7.40-7.27 (m, 7H), 7.01 (dd, J=9.0, 2.1 Hz, 1H), 5.67 (s, 1H), 3.79 (dd, J=12.7, 6.6 Hz, 2H), 3.17-3.10 (m, 4H), 3.00 (dd, J=13.8, 7.1 Hz, 2H), 1.79-1.72 (m, 4H), 1.59 (d, J=5.6 Hz, 2H). LC-MS m/z: 349.0 [M+H]⁺. HPLC Purity (214 nm): >99%; t_{R}=1.54 min.

N-phenethyl-6-(piperidin-1-yl)-1H-benzo[d]imidazole-1-carboxamide: ¹H NMR (500 MHz, CDCl₃) δ 8.15 (s, 1H), 7.60 (d, J=8.9 Hz, 1H), 7.37-7.27 (m, 4H), 7.25-7.19 (m, 2H), 7.03 (dd, J=8.9, 2.1 Hz, 1H), 5.98 (s, 1H), 3.79 (q, J=6.4 Hz, 2H), 3.12-3.06 (m, 4H), 3.01 (t, J=6.7 Hz, 2H), 1.78-1.67 (m, 4H), 1.62-1.51 (m, 2H). LC-MS m/z: 349.0 [M+H]⁺. HPLC Purity (214 nm): >95%; t_{R}=1.57 min.

### Examples 14a and 14b—5-Morpholino-N-phenethyl-1H-benzo[d]imidazole-1-carboxamide and 6-Morpholino-N-phenethyl-1H-benzo[d]imidazole-1-carboxamide

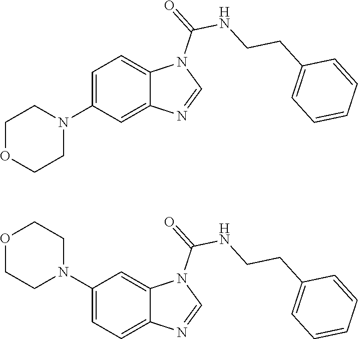 [SMILES: O=C(NCCc1ccccc1)n1cnc2cc(N3CCOCC3)ccc21 ; O=C(NCCc1ccccc1)n1cnc2ccc(N3CCOCC3)cc21]

To a solution of 5-bromo-1H-benzo[d]imidazole (3.9 g, 20 mmol) in THF (25 mL) was added NaH (1.6 g, 40 mmol) at 0° C. and the mixture was stirred at 0° C. for 30 min followed by the addition of PMBCl (4.1 g, 26 mmol). The solution was stirred at 60° C. for 3 h and poured into ice-water (20 mL). The mixture was extracted with EA (50 mL) and the organic layers were concentrated and purified by silica gel column chromatography (PE/EA=1/1) to give 5-bromo-1-(4-methoxybenzyl)-1H-benzo[d]imidazole (4.9 g, 78%) as a yellow solid. LC-MS m/z: 316.9 [M+H]⁺. HPLC Purity (214 nm): 98%; t_{R}=1.97 min.

Following general procedure H, 5-bromo-1-(4-methoxybenzyl)-1H-benzo[d]imidazole (632 mg, 2 mmol) and morpholine (522 mg, 6 mmol) afforded 4-(1-(4-methoxybenzyl)-1H-benzo[d]imidazol-5-yl)morpholine (210 mg, 32%) as a yellow solid. LC-MS m/z: 324.0 [M+H]⁺. HPLC Purity (214 nm): 82%; t_{R}=1.81 min.

A solution of 4-(1-(4-methoxybenzyl)-1H-benzo[d]imidazol-5-yl) morpholine (236 mg, 1.0 mmol) in TFA (5 mL) was stirred at 120° C. for 3 h under microwave conditions and concentrated. The mixture was purified by silica gel column chromatography (DCM/MeOH=10/1) to give 4-(1H-benzo[d]imidazol-5-yl) morpholine (80 mg, crude) as a yellow solid. LC-MS m/z: 204.0 [M+H]⁺. HPLC Purity (214 nm): 89%; t_{R}=1.42 min.

Following general procedure B, 4-(1H-benzo[d]imidazol-5-yl) morpholine (80 mg, 0.4 mmol) and (2-isocyanatoethyl)benzene (88 mg, 0.6 mmol) gave 5-morpholino-N-phenethyl-1H-benzo[d]imidazole-1-carboxamide (18 mg, 12.9%) and 6-morpholino-N-phenethyl-1H-benzo[d]imidazole-1-carboxamide (11 mg, 7.8%) as white solids.

5-morpholino-N-phenethyl-1H-benzo[d]imidazole-1-carboxamide: ¹H NMR (400 MHz, CDCl₃) δ 8.28 (s, 1H), 7.42-7.33 (m, 3H), 7.33-7.26 (m, 4H), 6.98 (dd, J=8.9, 2.3 Hz, 1H), 5.71 (s, 1H), 4.00-3.85 (m, 4H), 3.80 (dd, J=12.6, 6.6 Hz, 2H), 3.25-3.10 (m, 4H), 3.02 (t, J=6.7 Hz, 2H). LC-MS m/z: 351.3 [M+H]⁺. HPLC Purity (214 nm): 92.09%; t_{R}=7.12 min.

6-morpholino-N-phenethyl-1H-benzo[d]imidazole-1-carboxamide: ¹H NMR (400 MHz, CDCl₃) δ 8.11 (s, 1H), 7.66 (d, J=8.9 Hz, 1H), 7.36 (t, J=7.3 Hz, 2H), 7.28 (dd, J=7.8, 6.2 Hz, 3H), 7.20 (d, J=2.1 Hz, 1H), 7.00 (dd, J=8.9, 2.3 Hz, 1H), 5.67 (s, 1H), 3.95-3.84 (m, 4H), 3.78 (dd, J=25.3, 18.9 Hz, 2H), 3.24-3.09 (m, 4H), 3.02 (t, J=6.6 Hz, 2H). LC-MS m/z: 351.3 [M+H]⁺. HPLC Purity (214 nm): 95.87%; t_{R}=7.29 min.

### Examples 15a and 15b —5-(4-Methylpiperazin-1-yl)-N-phenethyl-1H-benzo[d]imidazole-1-carboxamide and 6-(4-Methylpiperazin-1-yl)-N-phenethyl-1H-benzo[d]imidazole-1-carboxamide

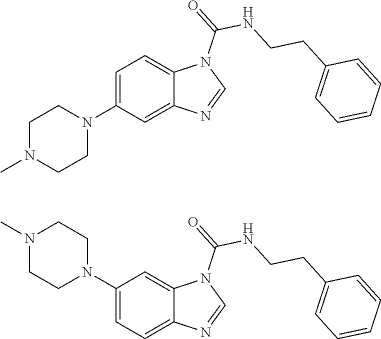 [SMILES: CN1CCN(c2ccc3c(c2)ncn3C(=O)NCCc2ccccc2)CC1 ; CN1CCN(c2ccc3ncn(C(=O)NCCc4ccccc4)c3c2)CC1]

Following general procedure H, 5-bromo-1-(4-methoxybenzyl)-1H-benzo[d]imidazole (632 mg, 2 mmol) and 1-methylpiperazine (600 mg, 6 mmol) afforded 1-(4-methoxybenzyl)-5-(4-methylpiperazin-1-yl)-1H-benzo[d]imidazole (510 mg, 76%) as a yellow solid. LC-MS m/z: 337.2 [M+H]⁺. HPLC Purity (214 nm): 79%; t_{R}=1.40 min.

A solution of 1-(4-methoxybenzyl)-5-(4-methylpiperazin-1-yl)-1H-benzo[d]imidazole (504 mg, 1.5 mmol) in TFA (5 mL) was stirred at 120° C. for 3 h under microwave conditions and concentrated. The mixture was purified by silica gel column chromatography (DCM/MeOH=10/1) to give 5-(4-methylpiperazin-1-yl)-1H-benzo[d]imidazole (440 mg, crude) as a yellow solid. LC-MS m/z: 217.1 [M+H]⁺. HPLC Purity (214 nm): 82%; t_{R}=1.45 min.

Following general procedure B, 5-(4-methylpiperazin-1-yl)-1H-benzo[d]imidazole (430 mg, 2 mmol) and (2-isocyanatoethyl)benzene (441 mg, 3.0 mmol) afforded 5-(4-methylpiperazin-1-yl)-N-phenethyl-1H-benzo[d]imidazole-1-carboxamide (59 mg, 8.1%) and 6-(4-methylpiperazin-1-yl)-N-phenethyl-1H-benzo[d]imidazole-1-carboxamide (69 mg, 9.5%) as white solids.

5-(4-methylpiperazin-1-yl)-N-phenethyl-1H-benzo[d]imidazole-1-carboxamide: ¹H NMR (400 MHz, CDCl₃) δ 8.27 (s, 1H), 7.41-7.26 (m, 7H), 6.99 (dd, J=9.0, 2.3 Hz, 1H), 5.71 (s, 1H), 3.79 (dd, J=12.6, 6.6 Hz, 2H), 3.33-3.18 (m, 4H), 3.01 (t, J=6.7 Hz, 2H), 2.73 (d, J=25.9 Hz, 4H), 2.43 (s, 3H). LC-MS m/z: 364.2 [M+H]⁺. HPLC Purity (214 nm): 96.20%; t_{R}=5.22 min.

6-(4-methylpiperazin-1-yl)-N-phenethyl-1H-benzo[d]imidazole-1-carboxamide: ¹H NMR (400 MHz, CDCl₃) δ 8.10 (s, 1H), 7.63 (d, J=8.9 Hz, 1H), 7.35 (t, J=7.2 Hz, 2H), 7.28 (t, J=4.4 Hz, 2H), 7.26 (s, 1H), 7.19 (d, J=2.2 Hz, 1H), 7.02 (dd, J=8.9, 2.3 Hz, 1H), 5.71 (s, 1H), 3.80 (dd, J=12.7, 6.4 Hz, 2H), 3.31-3.10 (m, 4H), 3.02 (t, J=6.7 Hz, 2H), 2.66-2.53 (m, 4H), 2.38 (s, 3H). LC-MS m/z: 364.2 [M+H]⁺. HPLC Purity (214 nm): 96.9%; t_{R}=5.72 min.

### Examples 16a and 16b—N-iso-Butyl-5-(pyrimidin-5-yl)-1H-benzo[d]imidazole-1-carboxamide and N-iso-Butyl-6-(pyrimidin-5-yl)-1H-benzo[d]imidazole-1-carboxamide

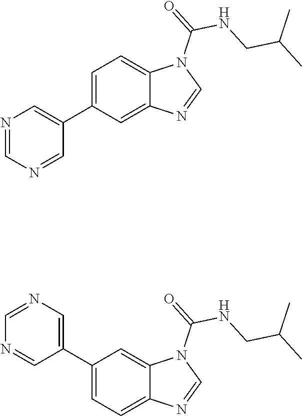 [SMILES: CC(C)CNC(=O)n1cnc2cc(-c3cncnc3)ccc21 ; CC(C)CNC(=O)n1cnc2ccc(-c3cncnc3)cc21]

Following general procedure D, 5-(pyrimidin-5-yl)-1H-benzo[d]imidazole (196 mg, 1.0 mmol) and 2-methylpropyl amine (150 mg, 2 mmol) afforded N-isobutyl-5-(pyrimidin-5-yl)-1H-benzo[d]imidazole-1-carboxamide (22 mg, 7.4%) and N-isobutyl-6-(pyrimidin-5-yl)-1H-benzo[d]imidazole-1-carboxamide (30 mg, 10.1%) as white solids.

N-isobutyl-5-(pyrimidin-5-yl)-1H-benzo[d]imidazole-1-carboxamide: ¹H NMR (400 MHz, CDCl₃) δ 9.23 (s, 1H), 9.02 (s, 2H), 8.47 (s, 1H), 8.18-7.95 (m, 2H), 7.61 (dt, J=40.4, 20.2 Hz, 1H), 5.84 (s, 1H), 3.40 (t, J=6.4 Hz, 2H), 2.02 (dt, J=13.5, 6.7 Hz, 1H), 1.07 (d, J=6.7 Hz, 6H). LC-MS m/z: 296.1 [M+H]⁺. HPLC Purity (214 nm): 100%; t_{R}=7.01 min.

N-isobutyl-6-(pyrimidin-5-yl)-1H-benzo[d]imidazole-1-carboxamide: ¹H NMR (400 MHz, CDCl₃) δ 9.23 (d, J=8.9 Hz, 1H), 9.02 (s, 2H), 8.42 (s, 1H), 8.19 (d, J=19.5 Hz, 1H), 7.96 (d, J=8.4 Hz, 1H), 7.57 (dt, J=21.4, 10.7 Hz, 1H), 5.90 (s, 1H), 3.38 (t, J=6.4 Hz, 2H), 2.15-1.91 (m, 1H), 1.06 (d, J=6.7 Hz, 6H). LC-MS m/z: 296.1 [M+H]⁺. HPLC Purity (214 nm): 97.73%; t_{R}=6.90 min.

### Example 17—N-Benzyl-1H-benzo[d]imidazole-1-carboxamide

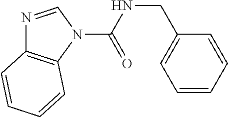 [SMILES: O=C(NCc1ccccc1)n1cnc2ccccc21]

Following general procedure B, 1H-benzo[d]imidazole (200 mg, 1.69 mmol) and (2-isocyanatomethyl)benzene (498 mg, 3.39 mmol) afforded the title compound (18.8 mg 4.4%) as a white solid. NMR (500 MHz, CDCl₃) δ 8.41 (s, 1H), 7.85-7.79 (m, 2H), 7.43-7.32 (m, 7H), 6.15 (s, 1H), 4.71 (d, J=5.6 Hz, 2H). LC-MS m/z: 252.1 [M+H]⁺. HPLC Purity (214 nm): >99%; t_{R}=1.74 min. ¹H

### Example 18—N-(2-Phenoxyethyl)-1H-benzo[d]imidazole-1-carboxamide

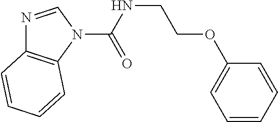 [SMILES: O=C(NCCOc1ccccc1)n1cnc2ccccc21]

Following general procedure C, 1H-benzo[d]imidazole (1.0 g, 8.5 mmol) and 4-nitrophenyl carbonochloridate (2.0 g 12.8 mmol) gave 4-nitrophenyl-1H-benzo[d]imidazole-1-carboxylate (1.0 g, 41%) as a white solid. LC-MS m/z: 284 [M+H]⁺. HPLC Purity (214 nm): 80%; t_{R}=1.98 min.

Following general procedure C, 4-nitrophenyl-1H-benzo[d]imidazole-1-carboxylate (60 mg, 0.42 mmol) and 2-phenoxyethanamine (100 mg, 0.35 mmol) afforded the title compound (9.0 mg, 8%) as a white solid. ¹H NMR (400 MHz, CDCl₃) δ 8.46 (s, 1H), 7.84 (dd, J=14.1, 7.2 Hz, 2H), 7.48-7.16 (m, 4H), 7.06-6.88 (m, 3H), 6.58 (s, 1H), 4.23 (t, J=5.0 Hz, 2H), 3.93 (dd, J=10.3, 5.3 Hz, 2H). LC-MS m/z: 282 [M+H]⁺. HPLC Purity (214 nm): 96%; t_{R}=1.89 min.

### Examples 19a and 19b—N-Phenethyl-1H-imidazo[4,5-b]pyridine-1-carboxamide and N-phenethyl-3H-imidazo[4,5-b]pyridine-3-carboxamide

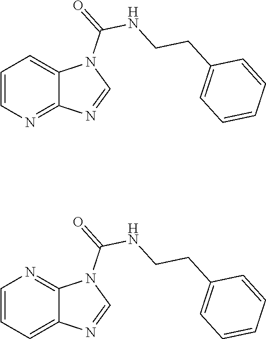 [SMILES: O=C(NCCc1ccccc1)n1cnc2ncccc21 ; O=C(NCCc1ccccc1)n1cnc2cccnc21]

Following general procedure B, 1H-imidazo[4,5-b]pyridine (596 mg, 5 mmol) and (isocyanatoethyl)benzene (883 mg, 1.2 mmol) afforded N-phenethyl-1H-imidazo[4,5-b]pyridine-1-carboxamide (81.9 mg, 1.5%) and N-phenethyl-3H-imidazo[4,5-b]pyridine-3-carboxamide (205 mg, 3.9%) as pale solids.

N-phenethyl-1H-imidazo[4,5-b]pyridine-1-carboxamide: ¹H NMR (400 MHz, CDCl₃) δ 8.77 (s, 1H), 8.44 (dd, J=4.8, 1.6 Hz, 1H), 8.31 (dd, J=8.0, 1.6 Hz, 1H), 8.18 (m, 1H), 7.30-7.19 (m, 6H), 3.84 (q, J=6.4 Hz, 2H), 3.02 (t, J=7.2 Hz, 2H). LC-MS m/z: 267.0 [M+H]⁺. HPLC Purity (214 nm): 98.6%; t_{R}=6.97 min.

N-phenethyl-3H-imidazo[4,5-b]pyridine-3-carboxamide: ¹H NMR (400 MHz, CDCl₃) δ 8.64 (s, 1H), 8.51 (dd, J=4.8, 1.6 Hz, 1H), 8.20 (dd, J=8.0, 1.6 Hz, 1H), 7.32-7.22 (m, 7H), 3.83 (q, J=7.2 Hz, 2H), 3.03 (t, J=7.2 Hz, 2H). LC-MS m/z: 267.3 [M+H]⁺. HPLC Purity (214 nm): 100%; t_{R}=7.32 min

### Example 20—N-Phenethyl-1H-imidazo[4,5-c]pyridine-1-carboxamide

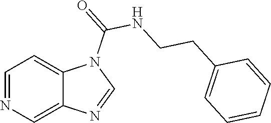 [SMILES: O=C(NCCc1ccccc1)n1cnc2cnccc21]

Following general procedure B, 1H-imidazo[4,5-c]pyridine (735 mg, 5.0 mmol) and (isocyanatoethyl)benzene (720 mg, 5.0 mmol) afforded the title compound (35 mg, 2.1%) as a white solid. ¹H NMR (400 MHz, CDCl₃) δ 8.96 (s, 1H), 8.36 (d, J=6.0 Hz, 2H), 7.62 (d, J=5.2 Hz, 1H), 7.34 (t, J=7.2 Hz, 2H), 7.26 (dd, J=17.7, 7.6 Hz, 3H), 6.88 (t, J=5.2 Hz, 1H), 3.79 (dd, J=12.6, 6.6 Hz, 2H), 3.02 (t, J=6.7 Hz, 2H). LC-MS m/z: 267.2 [M+H]⁺. HPLC Purity (214 nm): >99%; t_{R}=5.00 min.

### Example 21—N-Phenethyl-3H-imidazo[4,5-c]pyridine-3-carboxamide

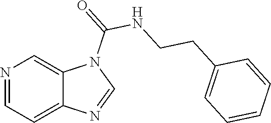 [SMILES: O=C(NCCc1ccccc1)n1cnc2ccncc21]

Following general procedure B, a solution of 1H-imidazo[4,5-c]pyridine (320 mg, 5.0 mmol) and (isocyanatoethyl)benzene (470 mg, 3.2 mmol) afforded the title compound (10 mg, 1.4%) as a white solid. ¹H NMR (400 MHz, CDCl₃) δ 8.99 (s, 1H), 8.55 (d, J=5.3 Hz, 1H), 8.42 (s, 1H), 7.73 (s, 1H), 7.38 (d, J=6.8 Hz, 2H), 7.30 (d, J=15.1 Hz, 3H), 5.78 (s, 1H), 3.84 (d, J=5.9 Hz, 2H), 3.04 (d, J=6.4 Hz, 2H). LC-MS m/z: 267.2[M+H]⁺. HPLC Purity (214 nm): >99%; t_{R}=5.06 min.

### Example 22—N-(2-(Pyridin-3-yl)ethyl)-1H-benzo[d]imidazole-1-carboxamide

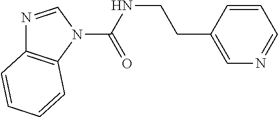 [SMILES: O=C(NCCc1cccnc1)n1cnc2ccccc21]

Following general procedure D, 1H-benzo[d]imidazole (500 mg, 4.28 mmol) and 2-(pyridin-3-yl)ethanamine (672 mg 5.51 mmol) afforded the title compound (64.1 mg 5.7%) as a white solid. ¹H NMR (500 MHz, CDCl₃) δ 8.44-8.40 (m, 2H), 8.37 (s, 1H), 7.85-7.74 (m, 2H), 7.58 (dt, J=7.8, 1.8 Hz, 1H), 7.38-7.33 (m, 2H), 7.24 (dd, J=7.9, 4.7 Hz, 1H), 6.94 (s, 1H), 3.78 (dd, J=12.8, 6.8 Hz, 2H), 3.03 (t, J=6.9 Hz, 2H). LC-MS m/z: 267.1 [M+H]⁺. HPLC Purity (214 nm): >99%, t_{R}=1.30 min.

### Example 23—N-Pentyl-1H-benzo[d]imidazole-1-carboxamide

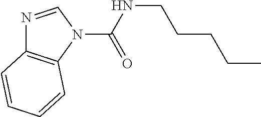 [SMILES: CCCCCNC(=O)n1cnc2ccccc21]

Following general procedure C, 4-nitrophenyl-1H-benzo[d]imidazole-1-carboxylate (150 mg, 0.53 mmol) and pentylamine (50 mg, 0.48 mmol) afforded the title compound (40.0 mg, 32%) as a white solid. ¹H NMR (400 MHz, CDCl₃) δ 8.43 (s, 1H), 7.83 (dd, J=9.6, 8.1 Hz, 2H), 7.46-0.33 (m, 2H), 5.82 (s, 1H), 3.58-3.48 (m, 2H), 1.60 (s, 2H), 1.54-1.37 (m, 4H), 0.94 (ddt, J=20.1, 13.8, 6.9 Hz, 3H). LC-MS m/z: 232 [M+H]⁺. HPLC Purity (214 nm): 99%; t_{R}=8.06 min.

### Example 24—N-Butyl-1H-benzo[d]imidazole-1-carboxamide

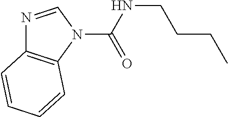 [SMILES: CCCCNC(=O)n1cnc2ccccc21]

Following general procedure D, 1H-benzo[d]imidazole (300 mg, 2.5 mmol) and butylamine (116 mg, 1.59 mmol) and the title compound (163.6 mg, 29.5%) as a colorless oil. ¹H NMR (400 MHz, CDCl₃) δ 8.42 (s, 1H), 7.82 (dd, J=18.9, 7.3 Hz, 2H), 7.39 (dq, J=7.4, 6.1 Hz, 2H), 5.72 (s, 1H), 3.54 (dd, J=12.9, 7.2 Hz, 2H), 1.70 (dt, J=15.0, 7.6 Hz, 2H), 1.47 (dd, J=15.1, 7.4 Hz, 2H), 1.00 (t, J=7.3 Hz, 3H). LC-MS m/z: 217.1 [M+H]⁺. HPLC Purity (214 nm): 96%; t_{R}=7.37 min.

### Examples 25a and 25b—5-Methoxy-N-phenethyl-1H-benzo[d]imidazole-1-carboxamide and 6-methoxy-N-phenethyl-1H-benzo[d]imidazole-1-carboxamide

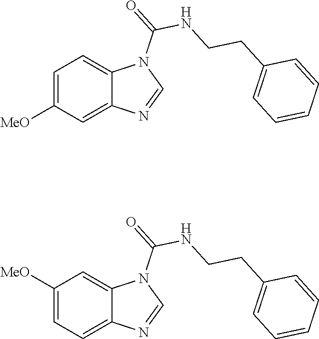 [SMILES: COc1ccc2c(c1)ncn2C(=O)NCCc1ccccc1 ; COc1ccc2ncn(C(=O)NCCc3ccccc3)c2c1]

Following general procedure B, 5-methoxy-1H-benzo[d]imidazole (500 mg, 3.37 mmol) and (2-isocyanatoethyl)benzene (744.9 mg, 5.06 mmol) afforded 5-methoxy-N-phenethyl-1H-benzo[d]imidazole-1-carboxamide (93.6 mg, 9.4%) and 6-methoxy-N-phenethyl-1H-benzo[d]imidazole-1-carboxamide (128.3 mg, 12.9%) as white solids.

5-methoxy-N-phenethyl-1H-benzo[d]imidazole-1-carboxamide: ¹H NMR (400 MHz, CDCl₃) δ 8.27 (s, 1H), 7.39-7.34 (m, 3H), 7.31-7.29 (m, 2H), 7.27-7.24 (m, 2H), 6.94-6.91 (m, 1H), 5.80 (s, 1H), 3.85 (s, 3H), 3.82-3.76 (m, 2H), 3.02 (t, J=6.8 Hz, 2H). LC-MS m/z: 296.2 [M+H]⁺. HPLC Purity (214 nm): 99.19%; t_{R}=8.09 min.

6-methoxy-N-phenethyl-1H-benzo[d]imidazole-1-carboxamide: ¹H NMR (400 MHz, CDCl₃) δ 8.12 (s, 1H), 7.64 (d, J=8.4 Hz, 1H), 7.37-7.35 (m, 2H), 7.34-7.29 (m, 3H), 7.26-7.25 (m, 1H), 6.97-6.94 (m, 1H), 5.87 (s, 3H), 3.82-3.76 (m, 2H), 3.02 (t, J=6.8 Hz, 2H). LC-MS m/z: 296.1 [M+H]⁺. HPLC Purity (214 nm): 1000%; t_{R}=8.13 min.

### Examples 26a and 26b—N-Phenethyl-5-(trifluoromethyl)-1H-benzo[d]imidazole-1-carboxamide and N-phenethyl-6-(trifluoromethyl)-1H-benzo[d]imidazole-1-carboxamide

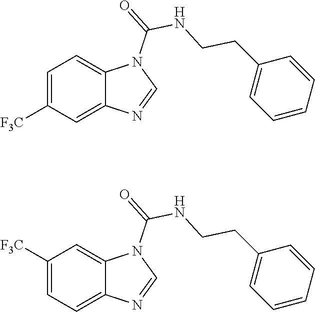 [SMILES: O=C(NCCc1ccccc1)n1cnc2cc(C(F)(F)F)ccc21 ; O=C(NCCc1ccccc1)n1cnc2ccc(C(F)(F)F)cc21]

Following general procedure G (method B), 4-(trifluoromethyl)benzene-1,2-diamine (500 mg, 2.84 mmol) afforded 6-(trifluoromethyl)-1H-benzo[d]imidazole (480 mg, 91%) as a brown solid. LC-MS m/z: 187.0 [M+H]⁺. HPLC purity (214 nm): 96%; t_{R}=1.48 min.

Following general procedure B, 6-(trifluoromethyl)-1H-benzo[d]imidazole (200 mg, 1.09 mmol) and (2-isocyanatoethyl)benzene (237 mg, 1.61 mmol) gave N-phenethyl-5-(trifluoromethyl)-1H-benzo[d]imidazole-1-carboxamide (48.8 mg, 15.1%) and N-phenethyl-6-(trifluoromethyl)-1H-benzo[d]imidazole-1-carboxamide (45.8 mg, 14.2%) as white solids.

N-phenethyl-5-(trifluoromethyl)-1H-benzo[d]imidazole-1-carboxamide: ¹H NMR (400 MHz, CDCl₃) δ 8.34 (s, 1H), 8.07 (s, 1H), 7.71 (d, J=8.4 Hz, 1H), 7.58 (d, J=8.8 Hz, 1H), 7.40-7.36 (m, 2H), 7.33-7.28 (m, 3H), 5.79 (s, 1H), 3.82 (q, J=6.4 Hz, 2H), 3.04 (t, J=6.8 Hz, 2H). LC-MS m/z: 334.1 [M+H]⁺. HPLC Purity (214 nm): 99%; t_{R}=9.43 min.

N-phenethyl-6-(trifluoromethyl)-1H-benzo[d]imidazole-1-carboxamide: ¹H NMR (400 MHz, DMSO-d₆) δ 8.36 (s, 1H), 8.03 (s, 1H), 7.88 (d, J=8.0 Hz, 1H), 7.61 (dd, J=8.4 Hz, 1.2 Hz, 1H), 7.39-7.37 (m, 2H), 7.36-7.28 (m, 3H), 5.76 (s, 1H), 3.82 (q, J=6.8 Hz, 2H), 3.04 (t, J=6.8 Hz, 2H). LC-MS m/z: 334.1 [M+H]⁺. HPLC Purity (214 nm): 98%; t_{R}=9.40 min.

### Example 27—4-Methoxy-N-phenethyl-1H-benzo[d]imidazole-1-carboxamide

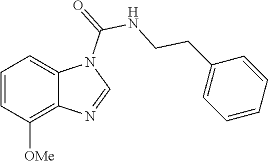 [SMILES: COc1cccc2c1ncn2C(=O)NCCc1ccccc1]

Following general procedure G (method B), 3-methoxybenzene-1,2-diamine (1.0 g, 7.24 mmol) afforded 4-methoxy-1H-benzo[d]imidazole (730 mg, 68%) as a brown solid. LC-MS m/z: 149.0 [M+H]⁺. HPLC Purity (214 nm): 99%; t_{R}=1.54 min.

Following general procedure B, 4-methoxy-1H-benzo[d]imidazole (200 mg, 1.35 mmol) and (2-isocyanatoethyl)benzene (300 mg, 2.03 mmol) afforded the title compound (326 mg, 81.9%) as a gray solid. ¹H NMR (400 MHz, CDCl₃) δ 8.25 (s, 1H), 7.38-7.36 (m, 2H), 7.34-7.25 (m, 3H), 7.23-7.21 (m, 1H), 7.07 (dd, J=4.4 Hz, 0.4 Hz, 1H), 6.77 (d, J=8.0 Hz, 1H), 5.85 (s, 1H), 4.01 (s, 3H), 3.80 (q, J=6.8 Hz, 2H), 3.02 (t, J=6.8 Hz, 2H). LC-MS m/z: 296.0 [M+H]⁺. HPLC Purity (214 nm): 99%; t_{R}=8.17 min.

### Example 28—2-Methoxy-6-morpholino-N-phenethyl-1H-benzo[d]imidazole-1-carboxamide

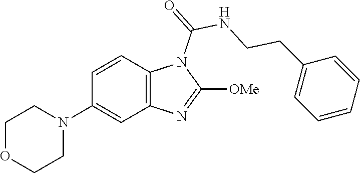 [SMILES: COc1nc2cc(N3CCOCC3)ccc2n1C(=O)NCCc1ccccc1]

A mixture of 4-fluoro-1,2-dinitrobenzene (840 mg, 4.5 mmol), morpholine (471 mg, 5.4 mmol) and K₂CO₃ (1.25 g, 9.0 mmol) in CH₃CN (10 mL) was stirred at 60° C. for 2 h. The reaction was concentrated and purified by silica gel column chromatography (DCM:MeOH=20:1) to give 4-(3,4-dinitrophenyl)morpholine (1.0 g, 88%) as a yellow solid. LC-MS m/z: 254.0 [M+H]⁺. Purity (214 nm): >87%; t_{R}=1.87 min.

Following general procedure F, 4-(3,4-dinitrophenyl)morpholine (1 g, 3.95 mmol) gave 4-morpholinobenzene-1,2-diamine (750 mg, 98%) as a yellow solid. LC-MS m/z: 194.1 [M+H]⁺. Purity (214 nm): >72%; t_{R}=1.29 min.

Following general procedure G (method A), 4-morpholinobenzene-1,2-diamine (750 mg, 3.9 mmol) afforded 4-(2-methoxy-1H-benzo[d]imidazole-6-yl)morpholine (100 mg, 11%) as a yellow oil. LC-MS m/z: 234.1 [M+H]⁺. Purity (214 nm): >77%; t_{R}=1.30 min.

Following general procedure B, 4-(2-methoxy-1H-benzo[d]imidazole-6-yl)morpholine (100 mg, 0.43 mmol) and (2-isocyanatoethyl)benzene (95 mg, 0.64 mmol) afforded the title compound (10.0 mg, 611%) as a white solid. ¹H NMR (400 MHz, CDCl₃) δ 7.82 (d, J=2.3 Hz, 1H), 7.36 (t, J=8.1 Hz, 3H), 7.32-7.20 (m, 3H), 6.98 (s, 1H), 6.90 (dd, J=8.7, 2.4 Hz, 1H), 4.11 (s, 3H), 3.93-3.82 (m, 4H), 3.72 (dd, J=12.5, 6.7 Hz, 2H), 3.23-3.13 (m, 4H), 2.96 (t, J=6.8 Hz, 2H). LC-MS m/z: 381.0 [M+H]⁺. HPLC: Purity (214 nm): >990%; t_{R}=7.30 min.

### Examples 29a and 29b —2-Methoxy-6-morpholino-N-phenethyl-1H-benzo[d]imidazole-1-carboxamide and 2-methoxy-5-morpholino-N-phenethyl-1H-benzo[d]imidazole-1-carboxamide

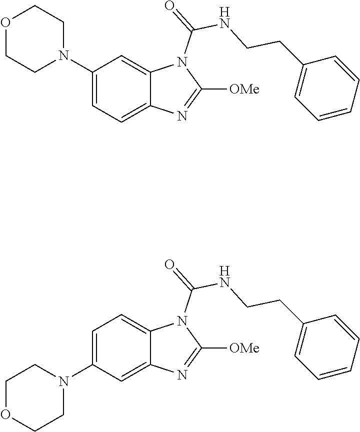 [SMILES: COc1nc2ccc(N3CCOCC3)cc2n1C(=O)NCCc1ccccc1 ; COc1nc2cc(N3CCOCC3)ccc2n1C(=O)NCCc1ccccc1]

Following general procedure B, 4-(2-methoxy-1H-benzo[d]imidazol-6-yl)morpholine (130 mg, 0.56 mmol) and (2-isocyanatobutyl)benzene (330 mg, 2.24 mmol) afforded 2-methoxy-6-morpholino-N-phenethyl-1H-benzo[d]imidazole-1-carboxamide (7.4 mg, 3.5%) and 2-methoxy-5-morpholino-N-phenethyl-1H-benzo[d]imidazole-1-carboxamide (27.7 mg, 13.0%) as yellow solids.

2-methoxy-6-morpholino-N-phenethyl-1H-benzo[d]imidazole-1-carboxamide: ¹H NMR (400 MHz, CDCl₃) δ 7.82 (d, J=2.4 Hz, 1H), 7.38-7.34 (m, 3H), 7.29-7.25 (m, 3H), 6.97 (t, J=4.8 Hz, 1H), 6.90 (dd, J=8.8, 2.8 Hz, 1H), 4.11 (s, 3H), 3.89-3.86 (m, 4H), 3.72 (q, J=5.6 Hz, 2H), 3.18-3.16 (m, 4H), 2.95 (t, J=6.8 Hz, 2H). LC-MS m/z: 381.2 [M+H]⁺. HPLC Purity (214 nm): 100%; t_{R}=9.01 min.

2-methoxy-5-morpholino-N-phenethyl-1H-benzo[d]imidazole-1-carboxamide: ¹H NMR (400 MHz, CDCl₃) δ 8.03 (d, J=8.8 Hz, 1H), 7.37-7.34 (m, 2H), 7.30-7.25 (m, 3H), 7.04 (d, J=2.4 Hz, 1H), 6.88 (dd, J=8.8, 2.4 Hz, 1H), 4.13 (s, 3H), 3.89-3.87 (m, 4H), 3.72 (q, J=5.6 Hz, 2H), 3.15-3.13 (m, 4H), 2.95 (t, J=6.8 Hz, 2H). LC-MS m/z: 381.4 [M+H]⁺. HPLC Purity (214 nm): 97.3%; t_{R}=7.65 min

### Examples 30a and 30b—2-Methoxy-N-phenethyl-5-(pyridin-3-yl)-1H-benzo[d]imidazole-1-carboxamide and 2-methoxy-N-phenethyl-6-(pyridin-3-yl)-1H-benzo[d]imidazole-1-carboxamide

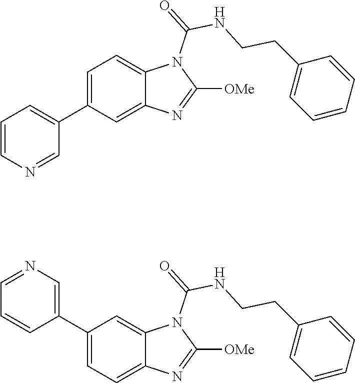 [SMILES: COc1nc2cc(-c3cccnc3)ccc2n1C(=O)NCCc1ccccc1 ; COc1nc2ccc(-c3cccnc3)cc2n1C(=O)NCCc1ccccc1]

Following general procedure G (method A), 4-bromobenzene-1,2-diamine (3.74 g, 20 mmol) afforded 6-bromo-2-methoxy-1H-benzo[d]-imidazole (3.4 g, 82%) as a brown solid. LC-MS m/z: 229.1 [M+H]⁺. HPLC Purity (214 nm): 100%; t_{R}=0.77 min.

Following general procedure A, 6-bromo-2-methoxy-1H-benzo[d]-imidazole (681 mg, 3.0 mmol) and pyridin-3-ylboronic acid (738 mg, 6.0 mmol) afforded 2-methoxy-6-(pyridin-3-yl)-1H-benzo[d]imidazole (348 mg, 51%) as a yellow solid. LC-MS m/z: 226.3 [M+H]⁺. HPLC Purity (214 nm): 92.4%; t_{R}=0.54 min.

Following general procedure B, 2-methoxy-6-(pyridin-3-yl)-1H-benzo[d]imidazole (200 mg, 0.89 mmol) and (2-isocyanatobutyl)benzene (144 mg, 0.98 mmol) afforded 2-methoxy-N-phenethyl-5-(pyridin-3-yl)-1H-benzo[d]imidazole-1-carboxamide (15 mg, 4.5%) and 2-methoxy-N-phenethyl-6-(pyridin-3-yl)-1H-benzo[d]imidazole-1-carboxamide (48.1 mg, 14.5%) as white solids.

2-methoxy-N-phenethyl-5-(pyridin-3-yl)-1H-benzo[d]imidazole-1-carboxamide: ¹H NMR (400 MHz, CDCl₃) δ 8.89 (s, 1H), 8.58 (d, J=4.4 Hz, 1H), 8.26 (d, J=8.0 Hz, 1H), 7.91 (dt, J=8.4, 2.0 Hz, 1H), 7.71 (d, J=2.0 Hz, 1H), 7.46 (dd, J=8.4, 2.0 Hz, 1H), 7.40-7.36 (m, 3H), 7.31-7.26 (m, 3H), 6.97 (t, J=4.8 Hz, 1H), 4.18 (s, 3H), 3.76 (q, J=5.6 Hz, 2H), 2.98 (t, J=6.8 Hz, 2H). LC-MS m/z: 373.3 [M+H]⁺. HPLC Purity (214 nm): 95.18%; t_{R}=6.33 min.

2-methoxy-N-phenethyl-6-(pyridin-3-yl)-1H-benzo[d]imidazole-1-carboxamide: ¹H NMR (400 MHz, CDCl₃) δ 8.90 (dd, J=2.4 Hz, 0.8 Hz, 1H), 8.57 (dd, J=4.8, 2.4 Hz, 1H), 8.44 (d, J=1.6 Hz, 1H), 7.95-7.92 (m, 1H), 7.57 (d, J=8.0 Hz, 1H), 7.49 (dd, J=8.4, 1.6 Hz, 1H), 7.39-7.37 (m, 3H), 7.36-7.26 (m, 3H), 6.93 (t, J=4.8 Hz, 1H), 4.18 (s, 3H), 3.75 (q, J=5.6 Hz, 2H), 2.98 (t, J=6.8 Hz, 2H). LC-MS m/z: 373.3 [M+H]⁺. HPLC Purity (254 nm): 98.3%; t_{R}=9.08 min

### Examples 31a and 31b—2-Methoxy-N-phenethyl-6-(pyrimidin-5-yl)-1H-benzo[d]imidazole-1-carboxamide and 2-methoxy-N-phenethyl-5-(pyrimidin-5-yl)-1H-benzo[d]imidazole-1-carboxamide

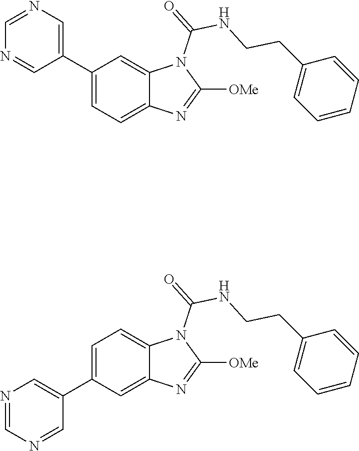 [SMILES: COc1nc2ccc(-c3cncnc3)cc2n1C(=O)NCCc1ccccc1 ; COc1nc2cc(-c3cncnc3)ccc2n1C(=O)NCCc1ccccc1]

Following general procedure A, 6-bromo-2-methoxy-1H-benzo[d]-imidazole (681 mg, 3.0 mmol) and pyridin-3-ylboronic acid (744 mg, 6.0 mmol) afforded 2-methoxy-6-(pyrimidin-5-yl)-1H-benzo[d]imidazole (325 mg, 48%) as a yellow solid. LC-MS m/z: 227.3 [M+H]⁺. HPLC Purity (214 nm): 88.6%; t_{R}=0.61 min.

Following general procedure B, 2-methoxy-6-(pyrimidin-5-yl)-1H-benzo[d]imidazole (279 mg, 1.23 mmol) and (2-isocyanatobutyl)benzene (271 mg, 1.84 mmol) afforded P1 (90 mg), 2-methoxy-N-phenethyl-6-(pyrimidin-5-yl)-1H-benzo[d]imidazole-1-carboxamide (50.9 mg, 11.1%) and 2-methoxy-N-phenethyl-5-(pyrimidin-5-yl)-1H-benzo[d]imidazole-1-carboxamide (13 mg, 2.8%) as white solids.

2-methoxy-N-phenethyl-6-(pyrimidin-5-yl)-1H-benzo[d]imidazole-1-carboxamide: ¹H NMR (500 MHz, CDCl₃) δ 9.19 (s, 1H), 8.99 (s, 2H), 8.30 (d, J=8.5 Hz, 1H), 7.70 (d, J=1.5 Hz, 1H), 7.45 (dd, J=8.5, 1.5 Hz, 1H), 7.39-7.37 (m, 2H), 7.31-7.26 (m, 3H), 6.97 (t, J=5.0 Hz, 1H), 4.18 (s, 3H), 3.76 (q, J=7.0 Hz, 2H), 2.98 (t, J=7.0 Hz, 2H). LC-MS m/z: 374.0 [M+H]⁺. HPLC Purity (214 nm): 94.4%; t_{R}=8.14 min.

2-methoxy-N-phenethyl-5-(pyrimidin-5-yl)-1H-benzo[d]imidazole-1-carboxamide: ¹H NMR (400 MHz, CDCl₃) δ 9.19 (s, 1H), 9.01 (s, 2H), 8.46 (d, J=1.2 Hz, 1H), 7.61 (d, J=4.4 Hz, 1H), 7.47 (dd, J=8.4, 2.0 Hz, 1H), 7.39-7.36 (m, 2H), 7.30-7.28 (m, 3H), 6.98 (t, J=4.8 Hz, 1H), 4.19 (s, 3H), 3.76 (q, J=6.8 Hz, 2H), 2.98 (t, J=6.8 Hz, 2H). LC-MS m/z: 374.2 [M+H]⁺. HPLC Purity (254 nm): 98.8%; t_{R}=8.20 min

### Examples 32a and 32b —3-Methyl-2-oxo-N-phenethyl-6-(pyrimidin-5-yl)-2,3-dihydro-1H-benzo[d]imidazole-1-carboxamide and 3-methyl-2-oxo-N-phenethyl-5-(pyrimidin-5-yl)-2,3-dihydro-1H-benzo[d]imidazole-1-carboxamide

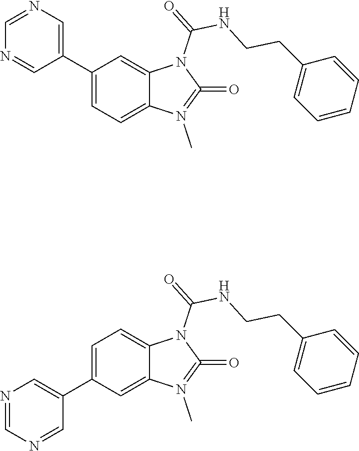

The first product (P1, 90 mg) from the reaction of Examples 31a and 31b was further purified by CHIRAL-HPLC to give 3-methyl-2-oxo-N-phenethyl-6-(pyrimidin-5-yl)-2,3-dihydro-1H-benzo[d]imidazole-1-carboxamide (20.3 mg, 4.4%) and 3-methyl-2-oxo-N-phenethyl-5-(pyrimidin-5-yl)-2,3-dihydro-1H-benzo[d]imidazole-1-carboxamide (27.2 mg, 5.9%) as white solids.

3-methyl-2-oxo-N-phenethyl-6-(pyrimidin-5-yl)-2,3-dihydro-1H-benzo[d]imidazole-1-carboxamide: ¹H NMR (400 MHz, CDCl₃) δ 9.20 (s, 1H), 8.98 (s, 2H), 8.79 (t, J=5.2 Hz, 1H), 8.53 (d, J=1.2 Hz, 1H), 7.44 (dd, J=8.0, 1.6 Hz, 1H), 7.36-7.32 (m, 2H), 7.26-7.23 (m, 3H), 7.14 (d, J=8.4 Hz, 1H), 3.70 (q, J=7.0 Hz, 2H), 3.48 (s, 3H), 2.97 (t, J=7.2 Hz, 2H). LC-MS m/z: 374.3 [M+H]⁺. HPLC Purity (214 nm): 100%; t_{R}=8.44 min.

3-methyl-2-oxo-N-phenethyl-5-(pyrimidin-5-yl)-2,3-dihydro-1H-benzo[d]imidazole-1-carboxamide: ¹H NMR (400 MHz, CDCl₃) δ 9.22 (s, 1H), 8.98 (s, 2H), 8.77 (t, J=5.6 Hz, 1H), 8.36 (d, J=8.4 Hz, 1H), 7.40 (dd, J=8.4, 2.0 Hz, 1H), 7.36-7.32 (m, 2H), 7.29-7.23 (m, 3H), 7.17 (d, J=1.6 Hz, 1H), 3.70 (q, J=7.0 Hz, 2H), 3.49 (s, 3H), 2.97 (t, J=7.2 Hz, 2H). LC-MS m/z: 374.4 [M+H]⁺. HPLC Purity (214 nm): 100%; t_{R}=8.55 min.

### Example 33—2-Methoxy-4-(1-methylpiperidin-4-yl)-N-phenethyl-1H-benzo[d]imidazole-1-carboxamide

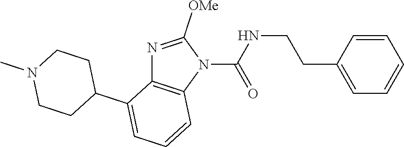 [SMILES: COc1nc2c(C3CCN(C)CC3)cccc2n1C(=O)NCCc1ccccc1]

To a solution of 2-bromo-6-nitroaniline (2.5 g, 11.52 mmol) and ammonium chloride (3.1 g, 57.60 mmol) in EtOH (75 mL) and H₂O (25 mL) was added Zn (3.8 g, 57.60 mmol). The mixture was stirred at 70° C. for 2 h and then filtered and purified by silica gel column chromatography (PE:EA=1:1) to give 3-bromobenzene-1,2-diamine (2.6 g, crude) as a brown solid/oil. LC-MS m/z: 186.9 [M+H]⁺. HPLC Purity (214 nm): 86%; t_{R}=1.73 min.

Following general procedure G (method A), 3-bromobenzene-1,2-diamine (2.6 g, 13.90 mmol) afforded 4-bromo-2-methoxy-1H-benzo[d]-imidazole (2.5 g, 78.9%) as an orange oil. LC-MS m/z: 228.9 [M+H]⁺. HPLC Purity (214 nm): 82%; t_{R}=1.77 min.

Following general procedure A, 4-bromo-2-methoxy-1H-benzo[d]imidazole (2.5 g, 11.01 mmol) and 1-methyl-4-(4,4,5,5-tetra-methyl-1,3,2-dioxaborolan-2-yl)-1,2,3,6-tetrahydropyridine (2.5 g, 11.01 mmol) afforded 2-methoxy-4-(1-methyl-1,2,3,6-tetrahydropyridin-4-yl)-1H-benzo[d]imidazole (2.9 g, 98%) as a brown solid. LC-MS m/z: 244.3 [M+H]⁺. HPLC Purity (214 nm): 90%; t_{R}=0.56 min.

To a solution of 2-methoxy-4-(1-methyl-1, 2, 3, 6-tetrahydropyridin-4-yl)-1H-benzo[d]-imidazole (2.34 g, 9.62 mmol) in MeOH (50 mL) was added PtO₂ (300 mg). The mixture was stirred at RT under H₂ for 4 h. The mixture was filtered and purified by silica gel column chromatography (DCM:MeOH=10:1) to give 2-methoxy-4-(1-methylpiperidin-4-yl)-1H-benzo[d]imidazole (1.8 g, 76%) as a brown solid. LC-MS m/z: 246.0 [M+H]⁺. HPLC Purity (214 nm): 80%; t_{R}=1.52 min.

Following general procedure B, 2-methoxy-4-(1-methylpiperidin-4-yl)-1H-benzo[d]imidazole (500 mg, 2.04 mmol) and (2-isocyanatoethyl)benzene (750 mg, 5.09 mmol) afforded the title compound (279 mg, 34%) as a white solid. ¹H NMR (400 MHz, CDCl₃) δ 8.06 (d, J=8.0 Hz, 1H), 7.36 (t, J=7.2 Hz, 2H), 7.31-7.27 (m, 1H), 7.25 (s, 2H), 7.20-7.06 (m, 2H), 6.98 (t, J=5.3 Hz, 1H), 4.30-4.10 (s, 3H), 3.72 (dd, J=12.5, 6.7 Hz, 2H), 3.66 (m, 2H), 3.27 (s, 1H), 3.05-2.76 (m, 9H), 2.05 (m, 2H). LC-MS m/z: 393.1 [M+H]⁺. HPLC Purity (214 nm): 95.3%; t_{R}=7.01 min.

### Example 34—2-Cyclopropoxy-N-phenethyl-1H-benzo[d]imidazole-1-carboxamide

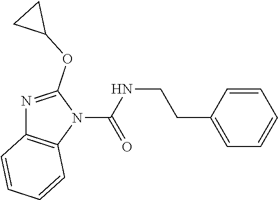 [SMILES: O=C(NCCc1ccccc1)n1c(OC2CC2)nc2ccccc21]

To a solution of 2-chloro-1H-benzo[d]imidazole (3 g, 19.7 mmol) in DMF (20 mL) at 0° C. was added NaH (1.18 g, 29.6 mmol). The mixture was stirred for 30 min followed by the addition of SEMCl (4.91 g, 29.6 mmol) and the mixture was stirred at RT for 15 h. The reaction was treated with water (150 mL), extracted with EA (3×100 mL) and the combined organic layers were concentrated. The residue was purified by silica gel column chromatography (PE:EA=4:1) to give 2-chloro-1-((2-(trimethylsilyl)ethoxy)methyl)-1H-benzo[d]imidazole (2.8 g, 50%) as a white solid. LC-MS m/z: 283.3 [M+H]⁺. HPLC Purity (254 nm): >99%; t_{R}=1.192 min.

A mixture of 2-chloro-1-((2-(trimethylsilyl)ethoxy)methyl)-1H-benzo[d]imidazole (400 mg, 1.42 mmol), cyclopropanol (329 mg, 5.67 mmol) and tBuONa (545 mg, 5.07 mmol) in DMF (10 mL) was stirred at RT for 15 h. The reaction was treated with water (50 mL), extracted with EA (3×30 mL) and the combined organic layers were concentrated. The residue was purified by silica gel column chromatography (PE:EA=3:1) to give 2-cyclopropoxy-1-((2-(trimethylsilyl)ethoxy)methyl)-1H-benzo[d]imidazole (220 mg, 45%) as a yellow solid. LC-MS m/z: 305.1 [M+H]⁺. HPLC Purity (254 nm): >96%; t_{R}=2.12 min.

2-Cyclopropoxy-1-((2-(trimethylsilyl)ethoxy)methyl)-1H-benzo[d]imidazole (220 mg, 0.72 mmol) in nBu₄NF (1M in THF, 5 mL) was stirred at 60° C. for 15 h. The reaction was concentrated and the residue was purified by silica gel column chromatography (DCM) to give 2-cyclopropoxy-1H-benzo[d]imidazole (100 mg, 79%) as a yellow oil. LC-MS m/z: 175.3 [M+H]⁺. HPLC Purity (254 nm): >97%; t_{R}=0.55 min.

Following general procedure B, 2-cyclopropoxy-1H-benzo[d]imidazole (100 mg, 0.47 mmol) and (2-isocyanatoethyl)benzene (169 mg, 1.15 mmol) afforded the title compound (43.5 mg, 23.6%) as a white solid. ¹H NMR (400 MHz, CDCl₃) δ 8.17 (d, J=6.6 Hz, 1H), 7.59-7.49 (m, 1H), 7.41-7.19 (m, 7H), 6.77 (s, 1H), 4.51 (dd, J=6.0, 3.1 Hz, 1H), 3.82-3.67 (m, 2H), 2.94 (t, J=6.4 Hz, 2H), 0.86 (dd, J=8.3, 4.7 Hz, 2H), 0.64 (s, 2H). LC-MS m/z: 322.0 [M+H]⁺. HPLC Purity (214 nm): >99%; t_{R}=2.10 min.

### Example 35—2-iso-Propoxy-N-phenethyl-1H-benzo[d]imidazole-1-carboxamide

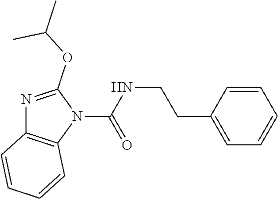 [SMILES: CC(C)Oc1nc2ccccc2n1C(=O)NCCc1ccccc1]

To a solution of propan-2-ol (6 mL) was added NaH (113 mg, 2.84 mmol) and the mixture was stirred at RT for 30 min. 2-chloro-1-((2-(trimethylsilyl)ethoxy)methyl)-1H-benzo[d]imidazole (400 mg, 1.42 mmol) was added and the mixture was stirred at 60° C. for 2 days. The reaction was treated with water (30 mL), extracted with EA (3×30 mL) and the combined organic layers were concentrated. The residue was purified by silica gel column chromatography (PE:EA=5:1) to give 2-isopropoxy-1-((2-(trimethylsilyl) ethoxy)methyl)-1H-benzo[d]imidazole (320 mg, 74%) as a colorless oil. LC-MS m/z: 307.2 [M+H]⁺. HPLC Purity (254 nm): >99%; t_{R}=1.96 min.

A solution of 2-isopropoxy-1-((2-(trimethylsilyl)ethoxy)methyl)-1H-benzo[d]imidazole (270 mg, 0.88 mmol) in nBu₄NF (1M in THF, 5 mL) was stirred at 60° C. for 15 h. The reaction was concentrated and the residue was purified by silica gel column chromatography (PE:EA=1:1) to give 2-isopropoxy-1H-benzo[d]imidazole (50 mg, 27%) as a yellow solid. LC-MS m/z: 177.1 [M+H]⁺. HPLC Purity (254 nm): >94%; t_{R}=1.37 min.

Following general procedure B, 2-isopropoxy-1H-benzo[d]imidazole (50 mg, 0.28 mmol) and (2-isocyanatoethyl)benzene (209 mg, 1.42 mmol) afforded the title compound (39.6 mg, 43.2%) as a white solid. ¹H NMR (400 MHz, CDCl₃) δ 8.21-8.15 (m, 1H), 7.49-7.44 (m, 1H), 7.38-7.31 (m, 2H), 7.29-7.18 (m, 5H), 7.08 (s, 1H), 5.41 (hept, J=6.2 Hz, 1H), 3.78 (dd, J=12.8, 6.0 Hz, 2H), 2.96 (t, J=6.6 Hz, 2H), 1.29 (d, J=6.2 Hz, 6H). LC-MS m/z: 324.0 [M+H]⁺. HPLC Purity (214 nm): >99%; t_{R}=2.21 min.

### Example 36—2-Ethoxy-N-phenethyl-1H-benzo[d]imidazole-1-carboxamide

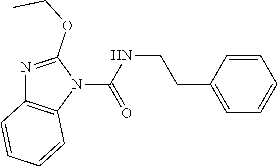 [SMILES: CCOc1nc2ccccc2n1C(=O)NCCc1ccccc1]

Following general procedure G (method A), benzene-1,2-diamine (800 mg, 7.4 mmol) afforded 2-ethoxy-1H-benzo[d]imidazole (800 mg, 66.7%) as a white solid. LC-MS m/z: 163.0 [M+H]⁺. HPLC Purity (214 nm): 42%; t_{R}=1.68 min.

Following general procedure B, 2-ethoxy-1H-benzo[d]imidazole (100 mg, 0.62 mmol) and (2-isocyanatoethyl)benzene (109 mg, 0.74 mmol) afforded the title compound (26 mg, 13.6%) as a white solid. ¹H NMR (400 MHz, CDCl₃) δ 8.19-8.16 (m, 1H), 7.48-7.45 (m, 1H), 7.37-7.33 (m, 2H), 7.29-7.19 (m, 5H), 7.04 (s, 1H), 4.59 (q, J=7.2 Hz, 2H), 3.76 (q, J=5.6 Hz, 2H), 2.96 (t, J=6.8 Hz, 2H), 1.31 (t, J=6.8 Hz, 3H). LC-MS m/z: 310.3 [M+H]⁺. HPLC Purity (214 nm): 100%; t_{R}=9.64 min.

### Example 37—N-Phenethyl-2-phenoxy-1H-benzo[d]imidazole-1-carboxamide

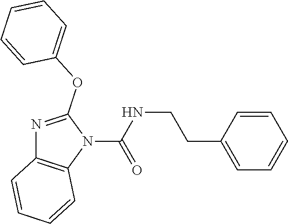 [SMILES: O=C(NCCc1ccccc1)n1c(Oc2ccccc2)nc2ccccc21]

A mixture of 2-chloro-1-((2-(trimethylsilyl)ethoxy)methyl)-1H-benzo[d]imidazole (400 mg, 1.4 mmol) and sodium phenolate (410 mg, 3.5 mmol) in dioxane (10 mL) was stirred at 100° C. for 2 days. The reaction was treated with water (50 mL), extracted with EA (2×50 mL) and the combined organic layers were concentrated. The residue was purified by silica gel column chromatography (PE:EA=5:1) to give 2-phenoxy-1-((2-(trimethylsilyl)ethoxy)methyl)-1H-benzo[d]imidazole (360 mg, 75%) as a yellow oil. LC-MS m/z: 341.4 [M+H]⁺. HPLC Purity (214 nm): >55%; t_{R}=1.25 min.

2-Phenoxy-1-((2-(trimethylsilyl) ethoxy)ethyl)-1H-benzo[d]imidazole (360 mg, 1.1 mmol) in nBu₄NF (1M in THF, 5 mL) was stirred at 60° C. for 15 h. The reaction was concentrated and the residue was purified by silica gel column chromatography (PE:EA=1:1) to give 2-phenoxy-1H-benzo[d]imidazole (170 mg, 77%) as a yellow solid. LC-MS m/z: 211.3 [M+H]⁺. HPLC Purity (214 nm): >50%; t_{R}=0.81 min.

Following general procedure B, 2-phenoxy-1H-benzo[d]imidazole (100 mg, 0.48 mmol) and (2-isocyanatoethyl) benzene (175 mg, 1.2 mmol) afforded the title compound (44.3 mg, 26.1%) as a white solid. ¹H NMR (400 MHz, CDCl₃) δ 8.25-8.18 (m, 1H), 7.51-7.43 (m, 3H), 7.37-7.31 (m, 1H), 7.30-7.12 (m, 9H), 7.05 (s, 1H), 3.79 (dd, J=12.3, 6.7 Hz, 2H), 2.98 (t, J=6.7 Hz, 2H). LC-MS m/z: 358.0 [M+H]⁺. HPLC Purity (214 nm): >96%; t_{R}=2.20.

### Examples 38a and 38b —5-Methoxy-6-morpholino-N-phenethyl-1H-benzo[d]imidazole-1-carboxamide and 6-methoxy-5-morpholino-N-phenethyl-1H-benzo[d]imidazole-1-carboxamide

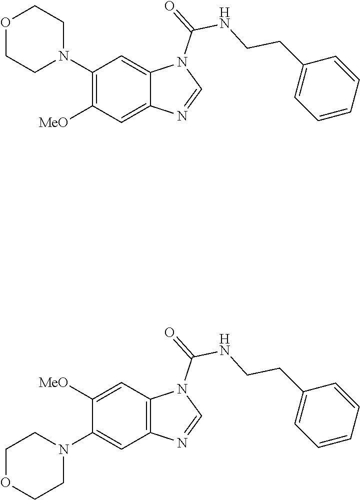 [SMILES: COc1cc2ncn(C(=O)NCCc3ccccc3)c2cc1N1CCOCC1 ; COc1cc2c(cc1N1CCOCC1)ncn2C(=O)NCCc1ccccc1]

A mixture of 6-bromo-5-methoxy-1H-benzo[d]imidazole (292 mg, 1.29 mmol), NaH (75 mg, 3.13 mmol) and THF (5 mL) was stirred at 0° C. for 1 h followed by the addition of 1-chloromethyl-4-methoxybenzene (231 mg, 1.481 mmol) and the reaction mixture was stirred for 16 h at 60° C. The mixture was then poured into water (20 mL) and extracted with EA (100 mL×3) and the combined organic layers were dried over Na₂SO₄, filtered and concentrated to give a residue which was purified by silica gel column chromatography (DCM:MeOH=15:1) to give 6-bromo-5-methoxy-1-(4-methoxybenzyl)-1H-benzo[d]imidazole (415 mg, 93.0%). LC-MS m/z: 347.2 [M+H]⁺. HPLC Purity (254 nm): >90%; t_{R}=0.88 min.

Following general procedure H, 6-bromo-5-methoxy-1-(4-methoxybenzyl)-1H-benzo[d]imidazole (415 mg, 1.20 mmol) and morpholine (850 mg, 9.77 mmol) afforded 4-(6-methoxy-3-(4-methoxybenzyl)-3H-benzo[d]imidazol-5-yl)morpholine (340 mg, 80.5%) as a brown solid. LC-MS m/z: 354.2 [M+H]⁺. HPLC Purity (254 nm): >39%; t_{R}=0.73 min.

A mixture of 4-(6-methoxy-3-(4-methoxybenzyl)-3H-benzo[d]imidazol-5-yl) morpholine (340 mg, 0.96 mmol) and TFA (8 mL) was stirred at 130° C. for 2 h under microwave conditions. Saturated aq. Na₂CO₃ solution (15 mL) was added to neutralize the TFA and the solution was extracted with DCM (100 mL×3). The mixture was concentrated to give a residue which was purified by silica gel column chromatography (DCM:MeOH=5:2) to give 4-(6-methoxy-3H-benzo[d]imidazol-5-yl)morpholine (120 mg, 53.5%) as a brown solid. LC-MS m/z: 234.2 [M+H]⁺. HPLC Purity (254 nm): >35%; t_{R}=0.54 min.

Following general procedure B, 4-(6-methoxy-3H-benzo[d]imidazol-5-yl)morpholine (115 mg, 0.494 mmol) and (2-isocyanatoethyl)benzene (73 mg, 0.495 mmol) afforded 5-methoxy-6-morpholino-N-phenethyl-1H-benzo[d]imidazole-1-carboxamide (41.3 mg, 22.0%) and 6-methoxy-5-morpholino-N-phenethyl-1H-benzo[d]imidazole-1-carboxamide (55.6 mg, 29.6%) as white solids.

5-methoxy-6-morpholino-N-phenethyl-1H-benzo[d]imidazole-1-carboxamide: ¹H NMR (500 MHz, CDCl₃) δ 8.14 (s, 1H), 7.31 (ddd, J=11.2, 8.9, 4.6 Hz, 5H), 7.22 (s, 2H), 5.88 (s, 1H), 3.90 (d, J=7.3 Hz, 7H), 3.80 (dd, J=12.6, 6.4 Hz, 2H), 3.08-2.96 (m, 6H). LC-MS m/z: 381.3 [M+H]⁺. HPLC Purity (214 nm): >95%; t_{R}=1.44 min.

6-methoxy-5-morpholino-N-phenethyl-1H-benzo[d]imidazole-1-carboxamide: ¹H NMR (500 MHz, CDCl₃) δ 8.05 (s, 1H), 7.38-7.27 (m, 5H), 7.26-7.24 (m, 1H), 5.72 (s, 1H), 3.92 (t, J=4.5 Hz, 5H), 3.87 (s, 2H), 3.79 (dd, J=12.6, 6.5 Hz, 2H), 3.11-3.05 (m, 4H), 3.02 (t, J=6.7 Hz, 2H). LC-MS m/z: 381.1 [M+H]⁺. HPLC Purity (214 nm): >99%; t_{R}=1.68 min.

### Example 39—4-Methoxy-6-morpholino-N-phenethyl-1H-benzo[d]imidazole-1-carboxamide

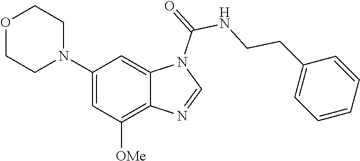 [SMILES: COc1cc(N2CCOCC2)cc2c1ncn2C(=O)NCCc1ccccc1]

To a solution of 2-methoxy-6-nitrobenzenamine (1.0 g, 0.60 mmol) in DCM (2.0 mL) was added NBS (0.11 g, 0.60 mmol). The mixture was stirred overnight at RT and purified by flash column chromatography to give 4-bromo-2-methoxy-6-nitrobenzenamine (1.20 g, 80%) as a brick-red solid. LC-MS m/z: 248.9 [M+H]⁺. HPLC Purity (254 nm): 95%; t_{R}=2.58 min.

To a solution of 4-bromo-2-methoxy-6-nitrobenzenamine (1.00 g, 4.0 mmol) in EtOH (15 mL) and H₂O (5.0 mL) was added NH₄Cl (1.10 g, 20.0 mmol) and Zn (1.30 g, 20.0 mmol) and the mixture was stirred at 70° C. for 2 h. The solution was cooled, extracted with EA/H₂O and concentrated under reduced pressure to give 5-bromo-3-methoxybenzene-1,2-diamine (853 mg, 3.89 mmol, 97%). LC-MS m/z: 219.0 [M+H]⁺. HPLC Purity (254 nm): 78%; t_{R}=1.54 min.

A solution of 5-bromo-3-methoxybenzene-1,2-diamine (5.00 g, 23.05 mmol) in AcOH (80 mL) was stirred at 105° C. for 2 h. The mixture was then partitioned between EtOAc and Na₂CO₃ solution, the organic layers were dried over Na₂SO₄, filtered and concentrated to give crude 6-bromo-4-methoxy-1H-benzo[d]imidazole (3.2 g, 60.8%). LC-MS m/z: 229.1[M+H]⁺. HPLC Purity (214 nm): 82%, t_{R}=0.72 min.

To a solution of 6-bromo-4-methoxy-1H-benzo[d]imidazole (0.80 g, 3.2 mmol) in THF (20 mL) was added NaH (0.15 g, 3.75 mmol) and the resulting reaction mixture was stirred at RT for 30 min. Then 4-methoxybenzylchloride (0.50 g, 3.2 mmol) was added and stirred 50° C. for 2 h. After workup the reaction mixture was purified by FCC (DCM:MeOH=15:1) to give 6-bromo-4-methoxy-1-(4-methoxybenzyl)-1H-benzo[d]imidazole (620 mg, 55.5%) as an off-white solid. LC-MS m/z: 349.1 [M+H]⁺. HPLC Purity (254 nm): 90%; t_{R}=1.15 min.

Following general procedure H, 6-bromo-4-methoxy-1-(4-methoxybenzyl)-1H-benzo[d]imidazole (0.70 g, 2.0 mmol) and morpholine (0.52 g, 6.0 mmol) afforded 4-(7-methoxy-3-(4-methoxybenzyl)-3H-benzo[d]imidazol-5-yl)morpholine (623 mg, 88.2%) as a white solid. LC-MS m/z: 354.2 [M+H]⁺. HPLC Purity (254 nm): 900%; t_{R}=0.74 min.

A solution of 4-(7-methoxy-3-(4-methoxybenzyl)-3H-benzo[d]imidazol-5-yl)morpholine (0.40 g, 1.27 mmol) in CF₃COOH (8 mL) was stirred under microwave at 125° C. for 3 h. The crude product was purified by silica gel column chromatography (DCM:MeOH=10:1) to give 4-(7-methoxy-3H-benzo[d]imidazol-5-yl)morpholine (100 mg, 38%) as a white solid. LC-MS m/z: 234.1 [M+H]⁺. HPLC Purity (254 nm): 95%; t_{R}=0.99 min.

Following general procedure B, 4-(7-methoxy-3H-benzo[d]imidazol-5-yl)morpholine (0.07 g, 0.30 mmol) and (2-isocyanatoethyl)benzene (0.05 g, 0.36 mmol) afforded the title compound (30.5 mg, 26.6%) as a white solid. ¹H NMR (400 MHz, CDCl₃) δ 8.03 (s, 1H), 7.35 (t, J=7.2 Hz, 2H), 7.27 (d, J=1.8 Hz, 2H), 6.77 (d, J=1.8 Hz, 1H), 6.44 (d, J=1.8 Hz, 1H), 5.71 (s, 1H), 4.00 (s, 2H), 3.90-3.84 (m, 3H), 3.80 (dd, J=12.6, 6.4 Hz, 2H), 3.14-3.07 (m, 3H), 3.01 (t, J=6.7 Hz, 2H). LC-MS m/z: 381.2 [M+H]⁺. HPLC Purity (214 nm): 100%; t_{R}=7.13 min.

### Example 40—4-Methoxy-5-morpholino-N-phenethyl-1H-benzo[d]imidazole-1-carboxamide

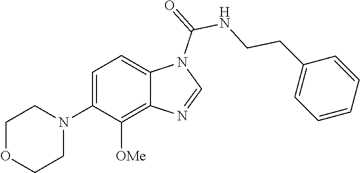 [SMILES: COc1c(N2CCOCC2)ccc2c1ncn2C(=O)NCCc1ccccc1]

To a solution of NaOH (10 g, 54 mmol) in H2O (400 mL) was added Br₂ (43.2 g, 270 mmol). The mixture was stirred at 50° C. for 3 h and slowly added to a solution of 2,4-dinitroaniline (10 g, 54 mmol) and KOH (4.6 g, 81 mmol) in MeOH (200 mL). Then the mixture was stirred at 50° C. for 30 min under N₂. The reaction mixture was cooled and filtered to give 5-bromo-4-methoxybenzo[c][1,2,5]oxadiazole 1-oxide (430 mg, crude) as a yellow solid. LC-MS m/z: 245.1 [M+H]⁺. HPLC Purity (214 nm): 89%; t_{R}=1.03 min.

A solution of crude 5-bromo-4-methoxybenzo[c][1,2,5]oxadiazole 1-oxide (1080 mg, 5 mmol), Pd(OH)₂ (150 mg, 0.8 mmol) in EA (10 mL) was stirred at RT for 15 h and filtered. The filtrate was concentrated to afford 4-bromo-3-methoxybenzene-1,2-diamine (900 mg, 83%) as a yellow solid. LC-MS m/z: 217.3 [M+H]⁺. HPLC Purity (214 nm): 79%; t_{R}=0.69 min.

A mixture of 4-bromo-3-methoxybenzene-1,2-diamine (864 mg, 4.0 mmol) in AcOH (10 mL) was stirred at 120° C. for 2 h under N₂ and concentrated. Sat. Na₂CO₃ solution (3 mL) was added to the residue and the mixture was extracted with DCM (10 mL). The organic layer was concentrated to give 6-bromo-7-methoxy-1H-benzo[d]imidazole (570 mg, crude) as a yellow solid. LC-MS m/z: 227.1 [M+H]⁺. HPLC Purity (214 nm): 71%; t_{R}=0.67 min.

To a solution of 6-bromo-7-methoxy-1H-benzo[d]imidazole (565 mg, 2.5 mmol) in THF (15 mL) was added NaH (200 mg, 5.0 mmol) at 0° C. The mixture was stirred at 0° C. for 30 min and PMBCl (515 mg, 3.3 mmol) was added. The solution was stirred at 60° C. for 3 hours and poured into ice-water (20 mL). The mixture was extracted with EA (50 mL) and the organic layers were concentrated and purified by silica gel column chromatography (PE/EA=1/1) to give 6-bromo-7-methoxy-1-(4-methoxybenzyl)-1H-benzo[d]imidazole (360 mg, crude) as a yellow solid. LC-MS m/z: 347.2 [M+H]⁺. HPLC Purity (214 nm): 70%; t_{R}=1.00 min.

Following general procedure H, 6-bromo-7-methoxy-1-(4-methoxybenzyl)-1H-benzo[d]imidazole (350 mg, 1 mmol) and morpholine (522 mg, 6 mmol) afforded 4-(7-methoxy-1-(4-methoxybenzyl)-1H-benzo[d]imidazol-6-yl)morpholine (510 mg, crude) as a yellow solid. LC-MS m/z: 354.2 [M+H]⁺. HPLC Purity (214 nm): 75%; t_{R}=0.76 min.

A solution of 4-(7-methoxy-1-(4-methoxybenzyl)-1H-benzo[d]imidazol-6-yl) morpholine (350 mg, 1.0 mmol) in TFA (5 mL) was stirred at 120° C. for 3 h under microwave conditions and concentrated. The mixture was purified by silica gel column chromatography (DCM/MeOH=10/1) to give 4-(7-methoxy-1H-benzo[d]imidazol-6-yl)morpholine (110 mg, crude) as a yellow solid. LC-MS m/z: 234.0 [M+H]⁺. HPLC Purity (214 nm): 75%; t_{R}=1.56 min.

Following general procedure B, 4-(7-methoxy-1H-benzo[d]imidazol-6-yl)morpholine (233 mg, 1.0 mmol) and (2-isocyanatoethyl)benzene (147 mg, 1.0 mmol) afforded the title compound (27.4 mg, 7.2%) as a white solid. ¹H NMR (400 MHz, CDCl₃) δ 8.23 (s, 1H), 7.44-7.26 (m, 5H), 7.03 (t, J=11.4 Hz, 1H), 6.94 (d, J=8.7 Hz, 1H), 5.70 (s, 1H), 4.29 (s, 3H), 3.97-3.87 (m, 4H), 3.79 (dd, J=12.6, 6.5 Hz, 2H), 3.15-3.05 (m, 4H), 3.01 (t, J=6.7 Hz, 2H). LC-MS m/z: 381.2 [M+H]⁺. HPLC Purity (214 nm): 95.76%; t_{R}=7.41 min.

### Example 41—N-(2-Morpholinoethyl)-1H-benzo[d]imidazole-1-carboxamide

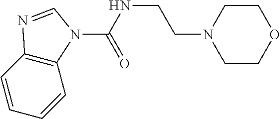 [SMILES: O=C(NCCN1CCOCC1)n1cnc2ccccc21]

Following general procedure C, 4-nitrophenyl 1H-benzo[d]imidazole-1-carboxylate (113 mg, 0.4 mmol) and 2-morpholinoethan-1-amine (47 mg, 0.36 mmol) afforded the title compound (26.7 mg, 24%) as a white solid. ¹H NMR (400 MHz, CDCl₃) δ 8.51 (s, 1H), 7.97-7.75 (m, 2H), 7.41 (dq, J=7.2, 6.0 Hz, 2H), 6.75 (s, 1H), 3.86-3.73 (m, 4H), 3.63 (dd, J=11.2, 5.0 Hz, 2H), 2.80-2.64 (m, 2H), 2.50-2.60 (m, 4H). LC-MS m/z: 275.0 [M+H]⁺. HPLC Purity (214 nm): 100%; t_{R}=6.47 min.

### Example 42—N-(2-(Benzyloxy)ethyl)-1H-benzo[d]imidazole-1-carboxamide

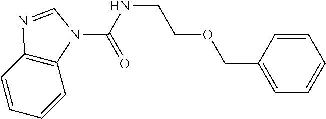 [SMILES: O=C(NCCOCc1ccccc1)n1cnc2ccccc21]

Following general procedure C, 4-nitrophenyl 1H-benzo[d]imidazole-1-carboxylate (142 mg, 0.5 mmol) and 2-(benzyloxy)ethan-1-amine (90 mg, 0.5 mmol) afforded the title compound (28.7 mg, 19.4%) as a clear oil. ¹H NMR (500 MHz, CDCl₃) δ 8.40 (s, 1H), 7.85-7.83 (m, 1H), 7.77 (s, 1H), 7.39-7.30 (m, 7H), 6.19 (bs, 1H), 4.59 (s, 2H), 3.76-3.72 (m, 4H). LC-MS m/z: 296.4 [M+H]⁺. HPLC Purity (214 nm): 95.5%; t_{R}=7.84 min.

### Example 43—N-(3-Phenoxypropyl)-1H-benzo[d]imidazole-1-carboxamide

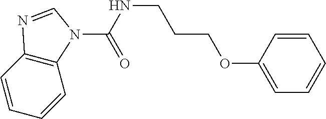 [SMILES: O=C(NCCCOc1ccccc1)n1cnc2ccccc21]

Following general procedure C, 4-nitrophenyl 1H-benzo[d]imidazole-1-carboxylate (113 mg, 0.4 mmol) and 3-phenoxypropan-1-amine (47 mg, 0.36 mmol) afforded the title compound (21.9 mg, 21%) as a white solid. ¹H NMR (400 MHz, CDCl₃) δ 8.45 (s, 1H), 8.04-7.79 (m, 2H), 7.42-7.35 (m, 2H), 7.30 (t, J=8.0 Hz, 2H), 7.00 (t, J=7.4 Hz, 1H), 6.90 (d, J=7.9 Hz, 2H), 6.69 (brs, 1H), 4.23 (t, J=5.4 Hz, 2H), 3.79 (dd, J=11.7, 5.6 Hz, 2H), 2.36-2.13 (m, 2H). LC-MS m/z: 296.1 [M+H]⁺. HPLC Purity (214 nm): 100%; t_{R}=7.94 min.

### Example 44—4-(1-Methylpiperidin-4-yl)-N-(3-phenylpropyl)-1H-benzo[d]imidazole-1-carboxamide

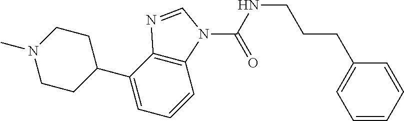 [SMILES: CN1CCC(c2cccc3c2ncn3C(=O)NCCCc2ccccc2)CC1]

Following general procedure D, 4-(1-methylpiperidin-4-yl)-1H-benzo[d]imidazole (100 mg, 0.47 mmol) and 3-phenylpropan-1-amine (57 mg, 0.42 mmol) afforded the title compound (40 mg 22%) as a white solid. ¹H NMR (400 MHz, CDCl₃) δ 8.57 (s, 1H), 7.65 (dd, J=20.4, 8.5 Hz, 1H), 7.38-7.13 (m, 7H), 6.20 (s, 1H), 3.56 (dt, J=13.4, 6.7 Hz, 5H), 3.29-2.66 (m, 7H), 2.36 (dd, J=23.4, 12.8 Hz, 2H), 2.20-1.88 (m, 4H). LC-MS m/z: 377 [M+H]⁺. HPLC Purity (214 nm): 99%; t_{R}=6.90 min.

### Example 45—4-(1-Methylpiperidin-4-yl)-N-(2-phenoxyethyl)-1H-benzo[d]imidazole-1-carboxamide

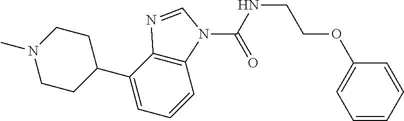 [SMILES: CN1CCC(c2cccc3c2ncn3C(=O)NCCOc2ccccc2)CC1]

Following general procedure D, 4-(1-methylpiperidin-4-yl)-1H-benzo[d]imidazole (100 mg, 0.47 mmol) and 2-phenoxyethanamine (57 mg, 0.42 mmol) afforded the title compound (60 mg, 34%) as a white solid. ¹H NMR (400 MHz, CDCl₃) δ 8.44 (s, 1H), 7.61 (d, J=8.0 Hz, 1H), 7.38-7.13 (m, 4H), 7.01-6.89 (m, 3H), 6.30 (s, 1H), 4.24 (t, J=5.0 Hz, 2H), 3.95 (dd, J=10.3, 5.3 Hz, 2H), 3.49 (s, 1H), 3.02 (d, J=11.7 Hz, 2H), 2.36 (s, 3H), 2.10-2.30 (m, 2H), 1.96-1.98 (m, 4H). LC-MS m/z: 379 [M+H]⁺. HPLC Purity (214 nm): 99%; t_{R}=1.30 min.

### Examples 46a and 46b—N-(2-Phenoxyethyl)-5-(trifluoromethyl)-1H-benzo[d]imidazole-1-carboxamide and N-(2-phenoxyethyl)-6-(trifluoromethyl)-1H-benzo[d]imidazole-1-carboxamide

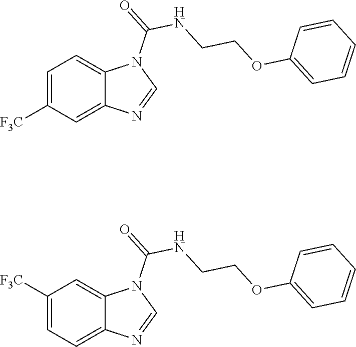 [SMILES: O=C(NCCOc1ccccc1)n1cnc2cc(C(F)(F)F)ccc21 ; O=C(NCCOc1ccccc1)n1cnc2ccc(C(F)(F)F)cc21]

Following general procedure C, 6-(trifluoromethyl)-1H-benzo[d]imidazole (120 mg, 0.65 mmol) and 2-phenoxyethan-1-amine (176 mg, 1.29 mmol) afforded N-(2-phenoxyethyl)-5-(trifluoromethyl)-1H-benzo[d]imidazole-1-carboxamide (52.0 mg, 27.7%) and N-(2-phenoxyethyl)-6-(trifluoromethyl)-1H-benzo[d]imidazole-1-carboxamide (40.5 mg, 21.6%) as white solids.

N-(2-phenoxyethyl)-5-(trifluoromethyl)-1H-benzo[d]imidazole-1-carboxamide: ¹H NMR (400 MHz, CDCl₃) δ 8.48 (s, 1H), 8.11 (s, 1H), 8.01 (d, J=8.8 Hz, 1H), 7.66 (d, J=8.4 Hz, 1H), 7.35-7.31 (m, 2H), 7.02 (t, J=7.2 Hz, 1H), 6.94 (d, J=8.0 Hz, 2H), 6.28 (s, 1H), 4.25 (t, J=4.8 Hz, 2H), 3.96 (q, J=5.2 Hz, 2H). LC-MS m/z: 350.0 [M+H]⁺. HPLC Purity (214 nm): 98%; t_{R}=9.24 min.

N-(2-phenoxyethyl)-6-(trifluoromethyl)-1H-benzo[d]imidazole-1-carboxamide: ¹H NMR (400 MHz, DMSO-d₆) δ 8.49 (s, 1H), 8.25 (s, 1H), 7.91 (d, J=8.4 Hz, 1H), 7.64 (dd, J=8.4, 0.8 Hz, 1H), 7.34-7.30 (m, 2H), 7.01 (t, J=7.6 Hz, 1H), 6.94 (d, J=8.0 Hz, 2H), 6.30 (s, 1H), 4.25 (t, J=4.8 Hz, 2H), 3.96 (q, J=5.2 Hz, 2H). LC-MS m/z: 350.0 [M+H]⁺. HPLC Purity (214 nm): 95%; t_{R}=9.26 min.

### Example 47—N-(2-(4-Fluorophenoxy)ethyl)-1H-benzo[d]imidazole-1-carboxamide

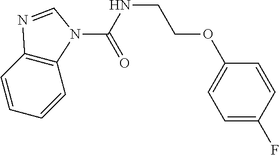 [SMILES: O=C(NCCOc1ccc(F)cc1)n1cnc2ccccc21]

Following general procedure C, 4-nitrophenyl 1H-benzo[d]imidazole-1-carboxylate (300 mg, 1.06 mmol) and 2-(4-fluorophenoxy)ethanamine (94 mg, 0.6 mmol) afforded the title compound (12.0 mg, 4%) as a white solid. ¹H NMR (400 MHz, CDCl₃) δ 8.46 (s, 1H), 7.90-7.77 (m, 2H), 7.26 (s, 2H), 6.93-6.81 (m, 4H), 6.36 (s, 1H), 4.20 (t, J=5.0 Hz, 2H), 3.94 (dd, J=10.3, 5.3 Hz, 2H). LC-MS m/z: 300 [M+H]⁺. HPLC Purity (214 nm): 96%; t_{R}=8.19 min.

### Example 48—N-(2-iso-Propoxyethyl)-1H-benzo[d]imidazole-1-carboxamide

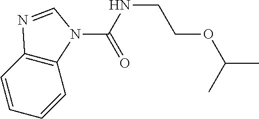 [SMILES: CC(C)OCCNC(=O)n1cnc2ccccc21]

Following general procedure C, 4-nitrophenyl 1H-benzo[d]imidazole-1-carboxylate (300 mg, 1.06 mmol) and 2-isopropoxyethanamine (80 mg, 0.7 mmol) afforded the title compound (26.0 mg, 10%) as a white solid. ¹H NMR (400 MHz, CDCl₃) δ 8.47 (s, 1H), 7.68 (s, 2H), 7.48-7.24 (m, 2H), 6.29 (s, 1H), 3.84-3.48 (m, 5H), 1.20 (dd, J=19.4, 6.0 Hz, 6H). LC-MS m/z: 248 [M+H]⁺. HPLC Purity (214 nm): 96%; t_{R}=6.90 min.

### Examples 49a and 49b—N-Butyl-5-(trifluoromethyl)-1H-benzo[d]imidazole-1-carboxamide and N-butyl-6-(trifluoromethyl)-1H-benzo[d]imidazole-1-carboxamide

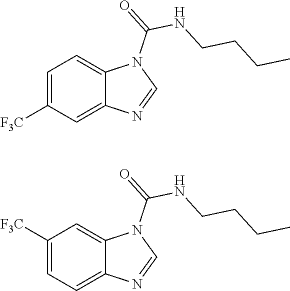 [SMILES: CCCCNC(=O)n1cnc2cc(C(F)(F)F)ccc21 ; CCCCNC(=O)n1cnc2ccc(C(F)(F)F)cc21]

Following general procedure C, 6-(trifluoromethyl)-1H-benzo[d]imidazole (140 mg, 0.75 mmol) and butylamine (99 mg, 1.35 mmol) afforded N-butyl-5-(trifluoromethyl)-1H-benzo[d]imidazole-1-carboxamide (43.1 mg, 20.0%) and N-butyl-6-(trifluoromethyl)-1H-benzo[d]imidazole-1-carboxamide (58.5 mg, 27.3%) as white solids.

N-butyl-5-(trifluoromethyl)-1H-benzo[d]imidazole-1-carboxamide: ¹H NMR (500 MHz, CDCl₃) δ 8.44 (s, 1H), 8.11 (s, 1H), 8.01 (d, J=8.5 Hz, 1H), 7.67 (d, J=7.2 Hz, 1.0 Hz, 1H), 5.68 (s, 1H), 3.57-3.53 (m, 2H), 1.74-1.68 (m, 2H), 1.51-1.44 (m, 2H), 1.01 (t, J=8.0 Hz, 3H). LC-MS m/z: 286.1 [M+H]⁺. HPLC Purity (214 nm): 99%; t_{R}=9.17 min.

N-butyl-6-(trifluoromethyl)-1H-benzo[d]imidazole-1-carboxamide: ¹H NMR (500 MHz, CDCl₃) δ 8.45 (s, 1H), 8.25 (s, 1H), 7.90 (d, J=8.5 Hz, 1H), 7.63 (dd, J=8.0 Hz, 1.0 Hz, 1H), 5.77 (s, 1H), 3.57-3.53 (m, 2H), 1.74-1.68 (m, 2H), 1.49-1.45 (m, 2H), 1.01 (t, J=6.5 Hz, 3H). LC-MS m/z: 286.1 [M+H]⁺. HPLC Purity (214 nm): 98%; t_{R}=9.19 min.

### Example 50—7-Methoxy-N-phenethyl-3H-imidazo[4,5-b]pyridine-3-carboxamide

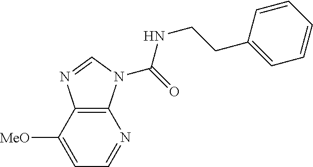 [SMILES: COc1ccnc2c1ncn2C(=O)NCCc1ccccc1]

To a solution of 4-chloro-3-nitropyridin-2-amine (500 mg, 2.89 mmol) in MeOH (8 mL) and DMF (4 mL) was added NaOMe (312 mg, 5.78 mmol). The mixture was stirred at RT for 3 h and then DCM (10 mL) was added to the reaction mixture, washed with water (20 mL×2) and extracted with DCM (20 mL×2). The combined organic layers were washed with brine (10 mL×1), dried over Na₂SO₄, filtered and concentrated to give 4-methoxy-3-nitropyridin-2-amine (480 mg, 98%) as a yellow solid. LC-MS m/z: 170 [M+H]⁺. HPLC Purity (214 nm): 93%; t_{R}=0.46 min.

To a mixed solution of 4-methoxy-3-nitropyridin-2-amine (480 mg, 2.84 mmol) in EA (8 mL) and MeOH (1 mL) was added 10% Pd/C (48 mg). The mixture was stirred at RT under H₂ overnight. The reaction mixture was filtered and concentrated to afford 4-methoxypyridine-2,3-diamine (400 mg, 99%) as a brown solid. LC-MS m/z: 140 [M+H]⁺. HPLC Purity (254 nm): 100%; t_{R}=0.37 min.

A solution of 4-methoxypyridine-2,3-diamine (200 mg, 1.44 mmol) in AcOH (10 mL) was stirred at 120° C. for 3 h. The reaction mixture was concentrated, and the residue was purified by silica gel column chromatography (DCM:MeOH=5:1) to afford 7-methoxy-3H-imidazo[4,5-b]pyridine (200 mg, 93%) as a yellow solid. LC-MS m/z: 150 [M+H]⁺. HPLC Purity (214 nm): 61%; t_{R}=1.25 min.

Following general procedure B, 7-methoxy-3H-imidazo[4,5-b]pyridine (200 mg, 1.34 mmol) and (2-isocyanatoethyl)benzene (218 mg, 1.47 mmol) afforded the title compound (20.7 mg, 5.2%) as a white solid. ¹H NMR (400 MHz, CDCl₃) δ 9.41 (s, 1H), 8.66 (s, 1H), 8.17 (d, J=6.0 Hz, 1H), 7.25-7.35 (m, 5H), 6.77 (d, J=5.6 Hz, 1H), 4.17 (s, 3H), 3.77 (q, J=6.0 Hz, 2H), 3.01 (t, J=7.2 Hz, 2H). LC-MS m/z: 297 [M+H]⁺. HPLC Purity (214 nm): 100%; t_{R}=8.59 min.

### Examples 51a and 51b—N-Butyl-5-methoxy-1H-benzo[d]imidazole-1-carboxamide and N-butyl-6-methoxy-1H-benzo[d]imidazole-1-carboxamide

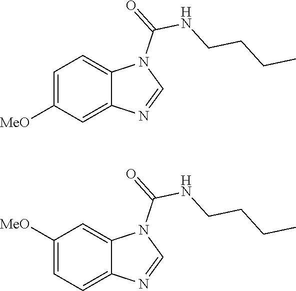 [SMILES: CCCCNC(=O)n1cnc2cc(OC)ccc21 ; CCCCNC(=O)n1cnc2ccc(OC)cc21]

Following general procedure C, 6-methoxy-1H-benzo[d]imidazole (700 mg, 4.72 mmol) and butylamine (518 mg, 7.09 mmol) afforded N-butyl-5-methoxy-1H-benzo[d]imidazole-1-carboxamide (168.3 mg, 14.4%) and N-butyl-6-methoxy-1H-benzo[d]imidazole-1-carboxamide (264.7 mg, 22.7%) as white solids.

N-butyl-6-methoxy-1H-benzo[d]imidazole-1-carboxamide: ¹H NMR (400 MHz, CDCl₃) δ 8.37 (s, 1H), 7.68 (d, J=8.8 Hz, 1H), 7.29 (d, J=2.4 Hz, 1H), 7.02 (dd, J=8.8, 2.4 Hz, 1H), 5.77 (brs, 1H), 3.87 (s, 3H), 3.54-3.49 (q, J=6.8 Hz, 2H), 1.72-1.67 (m, 2H), 1.51-1.43 (m, 2H), 0.99 (t, J=7.2 Hz, 3H). LC-MS m/z: 248.2 [M+H]⁺. HPLC Purity (214 nm): 100%; t_{R}=7.69 min.

N-butyl-5-methoxy-1H-benzo[d]imidazole-1-carboxamide: ¹H NMR (400 MHz, CDCl₃) δ 8.24 (s, 1H), 7.68 (d, J=8.8 Hz, 1H), 7.43 (d, J=2.4 Hz, 1H), 6.98 (dd, J=8.8, 2.4 Hz, 1H), 5.81 (brs, 1H), 3.87 (s, 3H), 3.54-3.49 (q, J=6.8 Hz, 2H), 1.71-1.65 (m, 2H), 1.49-1.44 (m, 2H), 1.01-0.98 (t, J=7.2 Hz, 3H). LC-MS m/z: 248.2 [M+H]⁺. HPLC Purity (214 nm): 98.18%; t_{R}=7.76 min.

### Example 52—N-Butyl-4-methoxy-1H-benzo[d]imidazole-1-carboxamide

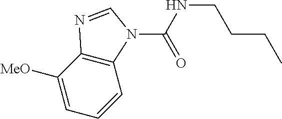 [SMILES: CCCCNC(=O)n1cnc2c(OC)cccc21]

Following general procedure C, 4-methoxy-1H-benzo[d]imidazole (80 mg, 0.54 mmol) and butylamine (99 mg, 1.35 mmol) afforded the title compound (71.5 mg, 53.0%) as a white solid. ¹H NMR (400 MHz, CDCl₃) δ 8.33 (s, 1H), 7.38 (dd, J=8.5 Hz, 1.0 Hz, 1H), 7.32 (t, J=8.5 Hz, 1H), 6.80 (d, J=8.0 Hz, 1H), 5.90 (s, 1H), 4.03 (s, 3H), 3.51 (dt, J=6.8 Hz, 2H), 1.68 (p, J=6.0 Hz, 2H), 1.47 (dt, J=6.0 Hz, 2H), 0.99 (t, J=7.5 Hz, 3H). LC-MS m/z: 248.1 [M+H]⁺. HPLC Purity (214 nm): 99%; t_{R}=7.77 min.

### Example 53—N-Phenethyl-6-(trifluoromethyl)-1H-imidazo[4,5-b]pyridine-1-carboxamide

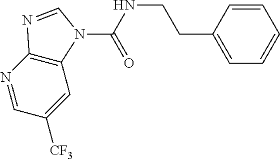 [SMILES: O=C(NCCc1ccccc1)n1cnc2ncc(C(F)(F)F)cc21]

A mixture of 5-(trifluoromethyl)pyridine-2,3-diamine (300 mg, 1.694 mmol) in AcOH (5 mL) was stirred at 120° C. overnight. The reaction mixture was cooled and basified with NaHCO₃(aq.) and then extracted with EA (20 mL×3). The combined organic layers were washed with brine (40 mL), dried over Na₂SO₄, filtered and concentrated to give 6-(trifluoromethyl)-3H-imidazo[4,5-b]pyridine (320 mg, 98%) as a gray solid/oil. LC-MS m/z: 187.9 [M+H]⁺. HPLC Purity (214 nm): 96.03%; t_{R}=1.61 min.

Following general procedure B, 6-(trifluoromethyl)-3H-imidazo[4,5-b]pyridine (200 mg, 1.069 mmol) and (2-isocyanatoethyl)benzene (314 mg, 2.138 mmol) afforded the title compound (109 mg, 30%) as a white solid. ¹H NMR (400 MHz, CDCl₃) δ 8.98 (s, 1H), 8.91 (s, 1H), 8.58 (d, J=1.1 Hz, 1H), 8.38 (d, J=1.7 Hz, 1H), 7.39-7.27 (m, 5H), 3.84 (dt, J=6.8 Hz, 2H), 3.03 (t, J=7.1 Hz, 2H). LC-MS m/z: 335.1 [M+H]⁺. HPLC Purity (214 nm): 96.01%; t_{R}=9.52 min.

### Example 54—4-(1-Methylpiperidin-4-yl)-N-phenethyl-6-(trifluoromethyl)-1H-benzo[d]imidazole-1-carboxamide

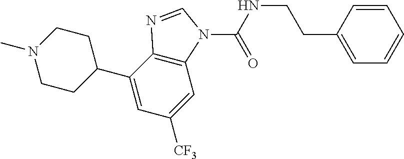 [SMILES: CN1CCC(c2cc(C(F)(F)F)cc3c2ncn3C(=O)NCCc2ccccc2)CC1]

A solution of 3-bromo-5-(trifluoromethyl)benzene-1,2-diamine (500 mg, 1.96 mmol) in AcOH (10 mL) was stirred at 120° C. for 3 h. The reaction was concentrated and sat. NaHCO₃ (30 mL) was added, extracted with EA (20 mL×3), washed with brine (50 mL×1), dried over Na₂SO₄ and concentrated to afford 4-bromo-6-(trifluoromethyl)-1H-benzo[d]imidazole (500 mg, 96%) as a brown solid. LC-MS m/z: 265 [M+H]⁺. HPLC Purity (214 nm): 97%; t_{R}=0.88 min.

Following general procedure A, 4-bromo-6-(trifluoromethyl)-1H-benzo[d]imidazole (500 mg, 1.89 mmol) and 1-methyl-4-(4,4,5,5-tetramethyl-1,3,2-dioxaborolan-2-yl)-1,2,3,6-tetrahydropyridine (507 mg, 2.27 mmol) afforded 4-(1-methyl-1,2,3,6-tetrahydropyridin-4-yl)-6-(trifluoromethyl)-1H-benzo[d]imidazole (330 mg, 62%) as a brown solid. LC-MS m/z: 282 [M+H]⁺. HPLC Purity (214 nm): 94%; t_{R}=0.65 min.

To a solution of 4-(1-methyl-1,2,3,6-tetrahydropyridin-4-yl)-6-(trifluoromethyl)-1H-benzo[d]imidazole (330 mg, 1.17 mmol) in MeOH (8 mL) was added Pd(OH)₂ (33 mg) and HCl/dioxane (1 drop). The mixture was stirred at 45° C. under H₂ overnight, filtered and concentrated to afford 4-(1-methylpiperidin-4-yl)-6-(trifluoromethyl)-1H-benzo[d]imidazole (300 mg, 91%) as a grey solid. LC-MS m/z: 284[M+H]⁺. HPLC purity (214 nm): 72%; t_{R}=1.71 min.

Following general procedure B, 4-(1-methylpiperidin-4-yl)-6-(trifluoromethyl)-1H-benzo[d]imidazole (200 mg, 0.71 mmol) and (2-isocyanatoethyl)benzene (114 mg, 0.78 mmol) afforded the title compound (28.1 mg, 9.2%) as a white solid. ¹H NMR (400 MHz, DMSO-d₆) δ 8.92-8.88 (m, 2H), 8.22 (s, 1H), 7.45 (s, 1H), 7.34-7.28 (m, 4H), 7.21-7.25 (m, 1H), 3.59-3.54 (m, 2H), 3.32-3.25 (m, 1H), 3.01-2.98 (m, 2H), 2.94-2.90 (m, 2H), 2.31 (s, 3H), 2.23-2.17 (m, 2H), 2.04-1.95 (m, 2H), 1.87-1.85 (m, 2H). LC-MS m/z: 431 [M+H]⁺. HPLC Purity (254 nm): 95.6%; t_{R}=7.33 min.

### Example 55—4-(1-Methylpiperidin-4-yl)-N-phenethyl-5-(trifluoromethyl)-1H-benzo[d]imidazole-1-carboxamide

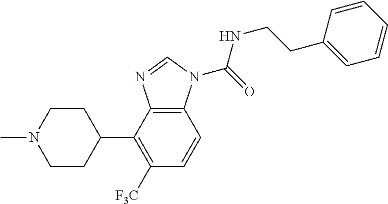 [SMILES: CN1CCC(c2c(C(F)(F)F)ccc3c2ncn3C(=O)NCCc2ccccc2)CC1]

To a solution of 2-chloro-5-(trifluoromethyl)aniline (30.00 g, 153.84 mmol) in CHCl₃ (300.00 mL) was added Br₂ (24.59 g, 153.84 mmol) at 50° C. during 30 min and the mixture was then cooled to RT and quenched with saturated Na₂CO₃ solution (300 mL). The organic phase was separated, dried over anhydrous Na₂SO₄, filtered and concentrated. The residue was purified by SGC (PE) to give 2-bromo-6-chloro-3-(trifluoromethyl)aniline (10.00 g, 23.6%) as a yellow oil. LC-MS m/z: no mass data. [M+H]⁺. HPLC Purity (214 nm): >85%; t_{R}=2.16 min.

Following general procedure A, 2-bromo-6-chloro-3-(trifluoromethyl)aniline (10.00 g, 36.43 mmol) and 1-methyl-4-(4,4,5,5-tetramethyl-1,3,2-dioxaborolan-2-yl)-1,2,3,6-tetrahydropyridine (8.94 g, 40.08 mmol) afforded 6-chloro-2-(1-methyl-1,2,3,6-tetrahydropyridin-4-yl)-3-(trifluoromethyl)aniline (6.0 g, 57.6%) as a yellow solid. LC-MS m/z: 291 [M+H]⁺. HPLC Purity (214 nm): >60%; t_{R}=1.45 min.

Following general procedure H, 6-chloro-2-(1-methyl-1,2,3,6-tetrahydropyridin-4-yl)-3-(trifluoromethyl)aniline (5.00 g, 17.20 mmol) and (4-methoxyphenyl)methanamine (3.54 g, 25.80 mmol) afforded 6-chloro-2-methyl-1,2,3,6-tetrahydropyridin-4-yl)-3-(trifluoromethyl)aniline (1.0 g, 15%) as a white solid. LC-MS m/z: 392.2 [M+H]⁺. HPLC Purity (214 nm): >60%; t_{R}=2.19 min.

Following general procedure (method B), 6-chloro-2-(1-methyl-1,2,3,6-tetrahydropyridin-4-yl)-3-(trifluoromethyl)aniline (1.5 g, 3.83 mmol) afforded 7-(1-methyl-1,2,3,6-tetrahydropyridin-4-yl)-6-(trifluoromethyl)-1H-benzo[d]imidazole (0.5 g, 50.0%) as a white solid. LC-MS m/z: 282.1 [M+H]⁺. HPLC Purity (214 nm): >80%; t_{R}=1.72 min.

A suspension of 7-(1-methyl-1,2,3,6-tetrahydropyridin-4-yl)-6-(trifluoromethyl)-11H-benzo[d]imidazole (0.50 g, 1.28 mmol) and Pd(OH)₂ (0.50 g) in MeOH (20.0 mL) was stirred at RT under H₂ atmosphere for 60 h. Then Pd/C was filtered off and the filtrate was concentrated. The residue was purified by SGC (DCM/MeOH=5/1) to give 7-(1-methylpiperidin-4-yl)-6-(trifluoromethyl)-1H-benzo[d]imidazole (0.3 g, 60% No) as a white solid. LC-MS m/z: 284.1 [M+H]⁺. HPLC Purity (214 nm): 100%; t_{R}=2.03 min.

Following general procedure B, 7-(1-methylpiperidin-4-yl)-6-(trifluoromethyl)-1H-benzo[d]imidazole (0.10 g, 0.35 mmol) and (2-isocyanatoethyl)benzene (0.06 g, 0.42 mmol) afforded the title compound (24.1 mg, 15.8%) as a white solid. ¹H NMR (400 MHz, CDCl₃) δ 8.47 (s, 1H), 7.80 (d, J=4.0 Hz, 1H), 7.60 (d, J=8.4 Hz, 1H), 7.37-7.33 (m, 3H), 7.29-7.28 (m, 2H), 3.78-3.74 (m, 2H), 3.02 (t, J=6.8 Hz, 3H), 2.74-2.69 (m, 3H), 1.91-1.68 (m, 4H). LC-MS m/z: 431.1 [M+H]⁺. HPLC Purity (214 nm): >97%; t_{R}=6.03 min.

### Example 56—N-Butyl-4-(1-methylpiperidin-4-yl)-5-(trifluoromethyl)-1H-benzo[d]imidazole-1-carboxamide

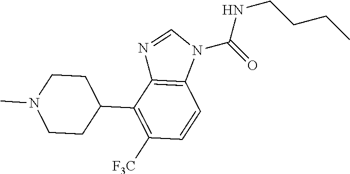 [SMILES: CCCCNC(=O)n1cnc2c(C3CCN(C)CC3)c(C(F)(F)F)ccc21]

Following general procedure B, 7-(1-methylpiperidin-4-yl)-6-(trifluoromethyl)-1H-benzo[d]imidazole (0.10 g, 0.35 mmol) and 1-isocyanatobutane (0.06 g, 0.42 mmol) afforded the title compound (65.7 mg, 48.6%) as a white solid. ¹H NMR (400 MHz, CDCl₃) δ 8.04 (d, J=8.0 Hz, 1H), 7.66 (d, J=8.8 Hz, 1H), 3.52-3.47 (m, 2H), 3.40-3.37 (m, 2H), 3.32-3.29 (m, 2H), 2.83-2.73 (m, 4H), 1.82-1.79 (m, 2H), 1.75-1.69 (m, 3H), 1.45 (dd, J=15.2 Hz, 7.2 Hz, 2H), 0.98 (t, J=7.2 Hz, 3H). LC-MS m/z: 383.1 [M+H]⁺. HPLC Purity (214 nm): >95%; t_{R}=5.80 min.

### Example 57—N-Butyl-4-(1-methylpiperidin-4-yl)-6-(trifluoromethyl)-1H-benzo[d]imidazole-1-carboxamide

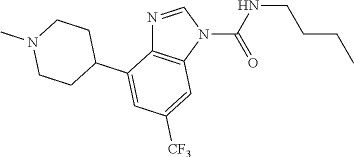 [SMILES: CCCCNC(=O)n1cnc2c(C3CCN(C)CC3)cc(C(F)(F)F)cc21]

Following general procedure B, 4-(1-methylpiperidin-4-yl)-6-(trifluoromethyl)-1H-benzo[d]imidazole (120 mg, 0.42 mmol) and 1-isocyanatobutane (63 mg, 0.64 mmol) afforded the title compound (38.7 mg, 24%) as a white solid. ¹H NMR (400 MHz, CDCl₃) J=8.78 (s, 1H), 8.64 (s, 1H), 8.37 (s, 1H), 7.34 (s, 1H), 3.49-3.42 (m, 5H), 2.78-2.74 (m, 2H), 2.70 (s, 3H), 2.27-2.24 (m, 2H), 2.09-2.06 (m, 2H), 1.68 (p, J=7.6 Hz, 2H), 1.45 (dq, J=14.5, 7.2 Hz, 2H), 0.97 (t, J=7.2 Hz, 3H). LC-MS m/z: 383 [M+H]⁺. HPLC Purity (254 nm): 93.1%; t_{R}=7.22 min.

### Example 58—N-Butyl-6-methoxy-4-(1-methylpiperidin-4-yl)-1H-benzo[d]imidazole-1-carboxamide

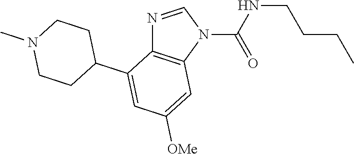 [SMILES: CCCCNC(=O)n1cnc2c(C3CCN(C)CC3)cc(OC)cc21]

A mixture of 2-bromo-4-methoxy-6-nitroaniline (984 mg, 4.0 mmol), Zn (1.04 g, 16 mmol) and NH₄Cl (1.0 g, 16 mmol) in EtOH/H₂O (20 mL, 2/1) was stirred at 80° C. for 2 h under N₂. The reaction mixture was cooled, filtered and concentrated in vacuo to give 3-bromo-5-methoxybenzene-1,2-diamine (900 mg, crude) as a yellow solid. LC-MS m/z: 217.2 [M+H]⁺. Purity (214 nm): 82%; t_{R}=0.74 min.

A mixture of 3-bromo-5-methoxybenzene-1,2-diamine (864 mg, 4.0 mmol) in AcOH (10 mL) was stirred at 120° C. for 2 h under N₂ and concentrated. Saturated Na₂CO₃ solution (3 mL) was added to the residue. The mixture was extracted with DCM (10 mL) and the organic layer was concentrated to give 4-bromo-6-methoxy-1H-benzo[d]imidazole (510 mg, crude) as a yellow solid. LC-MS m/z: 227.1 [M+H]⁺. HPLC purity (214 nm): 59%; t_{R}=0.66 min.

Following general procedure A, 4-bromo-6-methoxy-1H-benzo[d]imidazole (452 mg, 2.0 mmol) and 1-methyl-4-(4,4,5,5-tetramethyl-1,3,2-dioxaborolan-2-yl)-1,2,3,6-tetrahydropyridine (580 mg, 2.6 mmol) afforded 6-methoxy-4-(1-methyl-1,2,3,6-tetrahydropyridin-4-yl)-1H-benzo[d]imidazole (489 mg, crude) as a yellow solid. LC-MS m/z: 244.0 [M+H]⁺. Purity (214 nm): 91%; t_{R}=1.59 min.

A solution of 6-methoxy-4-(1-methyl-1,2,3,6-tetrahydropyridin-4-yl)-1H-benzo[d]imidazole (243 mg, 1.0 mmol), Pd(OH)₂ (50 mg, 0.2 mmol) in MeOH (10 mL) was stirred at 50° C. for 2 h and filtered. The filtrate was concentrated to afford 6-methoxy-4-(1-methylpiperidin-4-yl)-1H-benzo[d]imidazole (245 mg, 99%) as a yellow solid. LC-MS m/z: 246.0[M+H]⁺. Purity (214 nm): 79%; t_{R}=1.48 min.

Following general procedure B, 6-methoxy-4-(1-methylpiperidin-4-yl)-1H-benzo[d]imidazole (245 mg, 1 mmol) and 1-isocyanatobutane (99 mg, 1.0 mmol) afforded title compound (64.7 mg, 18.8%) as a white solid. ¹H NMR (400 MHz, CDCl₃) δ 8.62 (s, 1H), 7.40 (s, 2H), 6.77 (s, 1H), 3.86 (s, 3H), 3.50-3.32 (m, 5H), 2.83-2.65 (m, 2H), 2.69 (s, 3H), 2.29-2.13 (m, 2H), 2.10-2.05 (m, 2H), 1.81-1.56 (m, 2H), 1.45 (dq, J=14.5, 7.1 Hz, 2H), 0.98 (t, J=7.3 Hz, 3H). LC-MS m/z: 345.3 [M+H]⁺. HPLC Purity (214 nm): 95.56%; t_{R}=6.61 min.

### Example 59—N-(3-Phenylpropyl)-1H-imidazo[4,5-b]pyridine-1-carboxamide

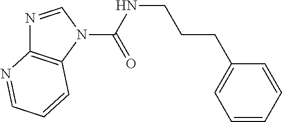 [SMILES: O=C(NCCCc1ccccc1)n1cnc2ncccc21]

Following general procedure C, 1H-imidazo[4,5-b]pyridine (400 mg, 3.36 mmol) and 3-phenylpropylamine (544 mg, 4.03 mmol) afforded title compound (351 mg, 37%) as a white solid. ¹H NMR (400 MHz, CDCl₃) δ 8.96 (s, 1H), 8.70 (t, J=5.6 Hz, 1H), 8.50 (dd, J=4.4, 1.2 Hz, 1H), 8.39 (dd, J=6.8, 1.2 Hz, 1H), 7.40 (dd, J=8.4, 4.8 Hz, 1H), 7.31-7.22 (m, 4H), 7.20-7.15 (m, 1H), 3.34 (q, J=6.4 Hz, 2H), 2.70 (t, J=7.6 Hz, 2H), 1.91 (t, J=7.6 Hz, 2H). LC-MS m/z: 281.1 [M+H]⁺. HPLC Purity (214 nm): 99%; t_{R}=7.59 min.

### Example 61—2-Methoxy-N-(4-phenylbutyl)-1H-imidazo[4,5-b]pyridine-1-carboxamide

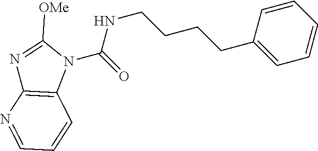 [SMILES: COc1nc2ncccc2n1C(=O)NCCCCc1ccccc1]

Following general procedure C, 2-methoxy-3H-imidazo[4,5-b]pyridine (280 mg, 1.88 mmol) and 4-phenylbutan-1-amine (504 mg, 3.38 mmol) afforded the title compound (32.0 mg, 5.3%) as a white solid. ¹H NMR (400 MHz, CDCl₃) δ 9.56 (s, 1H), 8.13 (dd, J=5.2 Hz, 1.2 Hz, 1H), 7.80 (dd, J=8.0 Hz, 1.2 Hz, 1H), 7.29-7.16 (m, 6H), 4.29 (s, 3H), 3.50 (q, J=6.4 Hz, 2H), 2.69 (t, J=6.8 Hz, 2H), 1.80-1.72 (m, 4H). LC-MS m/z: 325.0 [M+H]⁺. HPLC Purity (214 nm): 99%; t_{R}=9.47 min.

### Example 62—N-(2-Phenoxyethyl)-1H-imidazo[4,5-b]pyridine-1-carboxamide

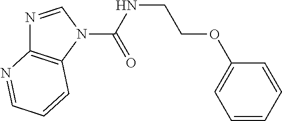 [SMILES: O=C(NCCOc1ccccc1)n1cnc2ncccc21]

Following general procedure C, 1H-imidazo[4,5-b]pyridine (200 mg, 1.68 mmol) and 2-phenoxyethanamine (276 mg, 2.02 mmol) afforded the title compound (150 mg, 37%) as a white solid. ¹H NMR (400 MHz, CDCl₃) δ 9.00 (s, 1H), 8.99 (t, J=5.2 Hz, 1H), 8.51 (dd, J=4.8, 1.2 Hz, 1H), 8.40 (dd, J=8.0, 1.2 Hz, 1H), 7.41 (dd, J=8.4, 4.8 Hz, 1H), 7.30 (t, J=8.0 Hz, 2H), 6.98 (d, J=8.8 Hz, 2H), 6.94 (d, J=7.2 Hz, 1H), 4.20 (t, J=5.6 Hz, 2H), 3.71 (q, J=5.6 Hz, 2H). LC-MS m/z: 283.1 [M+H]⁺. HPLC Purity (214 nm): 98%; t_{R}=7.13 min.

### Example 63—5-Fluoro-N-phenethyl-1H-benzo[d]imidazole-1-carboxamide

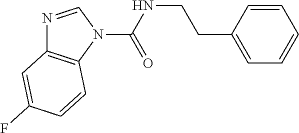 [SMILES: O=C(NCCc1ccccc1)n1cnc2cc(F)ccc21]

Following general procedure B, 5-fluoro-1H-benzo[d]imidazole (300 mg, 2.2 mmol) and (2-isocyanatoethyl)benzene (487 mg, 3.3 mmol) afforded the title compound (45.4 mg, 7.4%) as an orange solid. ¹H NMR (400 MHz, CDCl₃) δ 8.28 (s, 1H), 7.52 (dd, J=9.0, 4.6 Hz, 1H), 7.47 (dd, J=8.8, 2.5 Hz, 1H), 7.38 (t, J=7.2 Hz, 2H), 7.34-7.27 (m, 3H), 7.07 (td, J=9.0, 2.5 Hz, 1H), 5.66 (s, 1H), 3.80 (dd, J=12.5, 6.7 Hz, 2H), 3.02 (t, J=6.7 Hz, 2H). LC-MS m/z: 283.3 [M+H]⁺. HPLC Purity (214 nm): 100%; t_{R}=8.95 min.

### Example 64—4-Fluoro-N-phenethyl-1H-benzo[d]imidazole-1-carboxamide

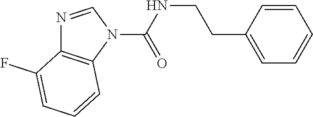 [SMILES: O=C(NCCc1ccccc1)n1cnc2c(F)cccc21]

Following general procedure B, 4-fluoro-1H-benzo[d]imidazole (140 mg, 1.0 mmol) and (2-isocyanatoethyl)benzene (140 mg, 1.2 mmol) afforded the title compound (66.3 mg, 22.7%) as a white solid. ¹H NMR (400 MHz, CDCl₃) δ 8.23 (s, 1H), 7.46-7.38 (m, 1H), 7.36-7.29 (m, 2H), 7.28 (d, J=4.6 Hz, 1H), 7.26-7.21 (m, 3H), 7.04 (dd, J=9.6, 8.2 Hz, 1H), 6.31 (s, 1H), 3.80 (dd, J=12.6, 6.8 Hz, 2H), 3.01 (t, J=6.8 Hz, 2H). LC-MS m/z: 284.0 [M+H]⁺. HPLC Purity (214 nm): 100%; t_{R}=2.03 min.

### Example 65—5-Fluoro-N-phenethyl-1H-benzo[d]imidazole-1-carboxamide

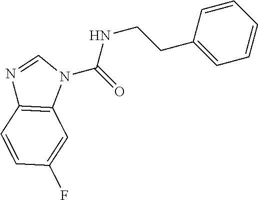 [SMILES: O=C(NCCc1ccccc1)n1cnc2ccc(F)cc21]

Following general procedure B, 5-fluoro-1H-benzo[d]imidazole (300 mg, 2.2 mmol) and (2-isocyanatoethyl) benzene (487 mg, 3.3 mmol) afforded the title compound (44.7 mg, 7.4%) as a white solid. ¹H NMR (400 MHz, CDCl₃) δ 8.21 (s, 1H), 7.70 (dd, J=8.9, 4.9 Hz, 1H), 7.40-7.33 (m, 3H), 7.29 (dd, J=15.2, 6.6 Hz, 3H), 7.08 (ddd, J=8.9, 7.9, 3.3 Hz, 1H), 5.74 (s, 1H), 3.79 (dt, =12.7, 6.5 Hz, 2H), 3.02 (t, J=6.7 Hz, 2H). LC-MS m/z: 283.3 [M+H]⁺. HPLC Purity (214 nm): 98.74%; t_{R}=8.42 min.

### Examples 66a and 66b—N-Phenethyl-5-(trifluoromethyl)-3H-imidazo[4,5-b]pyridine-3-carboxamide and N-phenethyl-5-(trifluoromethyl)-1H-imidazo[4,5-b]pyridine-1-carboxamide

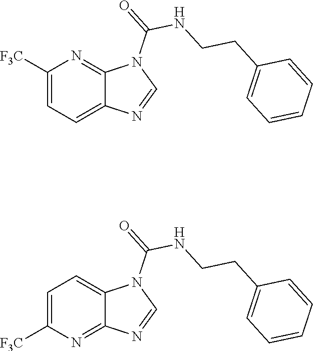 [SMILES: O=C(NCCc1ccccc1)n1cnc2ccc(C(F)(F)F)nc21 ; O=C(NCCc1ccccc1)n1cnc2nc(C(F)(F)F)ccc21]

A mixture of 6-(trifluoromethyl)pyridin-2-amine (2.0 g, 12.0 mmol and KNO₃ (1.3 g, 13.6 mmol) in H₂SO₄ (20 mL) was stirred at RT for 2 h. Ice was added and the resulting solid was collected by filtration. The solid was dissolved in H₂SO₄ (5 mL) and the mixture was stirred at 50° C. for 2 h. Additional ice was added and the solution filtered to give 3-nitro-6-(trifluoromethyl) pyridin-2-amine (660 mg, 23%) as a yellow solid. LC-MS m/z: 208 [M+H]⁺. HPLC Purity (214 nm): 70%; t_{R}=0.94 min.

To a solution of 3-nitro-6-(trifluoromethyl)pyridin-2-amine (600 mg, 3.0 mmol) in MeOH (50 mL) was added Pd/C (100 mg). The mixture was stirred at RT under H2 pressure for 2 h and then the mixture was filtered and concentrated to afford 6-(trifluoromethyl) pyridine-2,3-diamine (500 mg, 94%) as a yellow oil. LC-MS m/z: 178 [M+H]⁺. HPLC Purity (214 nm): 60%; t_{R}=1.66 min.

The solution of 6-(trifluoromethyl)pyridine-2,3-diamine (500 mg, 2.82 mmol) in AcOH (10 mL) was stirred at 120° C. for 120 min. Then the mixture was cooled and concentrated. The residue was purified by silica gel column chromatography (PE/EA=30:1) to give 5-(trifluoromethyl)-3H-imidazo[4,5-b]pyridine (100 mg, 80%) as a yellow solid. LC-MS m/z: 188 [M+H]⁺. HPLC Purity (214 nm): 80%; t_{R}=1.51 min.

Following general procedure D, 5-(trifluoromethyl)-3H-imidazo[4,5-b]pyridine (100 mg, 0.53 mmol) and 2-phenylethanamine (57 mg, 0.42 mmol) afforded N-phenethyl-5-(trifluoromethyl)-3H-imidazo[4,5-b]pyridine-3-carboxamide (60 mg 30%) and N-phenethyl-5-(trifluoromethyl)-1H-imidazo[4,5-b]pyridine-1-carboxamide (20 mg 10%) as white solids.

N-phenethyl-5-(trifluoromethyl)-3H-imidazo[4,5-b]pyridine-3-carboxamide: ¹H NMR (400 MHz, CDCl₃) δ 8.94 (s, 1H), 8.71 (s, 1H), 8.29 (d, J=8.3 Hz, 1H), 7.73 (t, J=10.8 Hz, 1H), 7.40-7.04 (m, 5H), 3.84 (dd, J=12.5, 6.9 Hz, 2H), 2.78 (t, J=7.1 Hz, 2H). LC-MS m/z: 335 [M+H]⁺. HPLC Purity (214 nm): 96%; t_{R}=9.44 min.

N-phenethyl-5-(trifluoromethyl)-1H-imidazo[4,5-b]pyridine-1-carboxamide: ¹H NMR (400 MHz, CDCl₃) δ 8.62 (s, 1H), 8.39 (d, J=8.4 Hz, 1H), 7.71 (d, J=8.4 Hz, 1H), 7.29-7.19 (m, 4H), 7.19-6.93 (m, 2H), 3.85 (dd, J=12.7, 6.7 Hz, 2H), 3.02 (t, J=6.8 Hz, 2H). LC-MS m/z: 335 [M+H]⁺. HPLC Purity (214 nm): 96%; t_{R}=8.70 min.

### Example 67—N-Butyl-5-fluoro-1H-benzo[d]imidazole-1-carboxamide

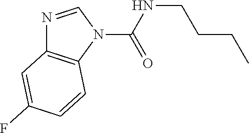 [SMILES: CCCCNC(=O)n1cnc2cc(F)ccc21]

Following general procedure B, 5-fluoro-1H-benzo[d]imidazole (200 mg, 1.5 mmol) and 1-isocyanatobutane (219 mg, 2.2 mmol) afforded the title compound (15 mg, 4.3%) as a white solid. ¹H NMR (400 MHz, CDCl₃) δ 8.38 (s, 1H), 7.82 (dd, J=9.0, 4.6 Hz, 1H), 7.53-7.46 (m, 1H), 7.16 (td, J=9.0, 2.5 Hz, 1H), 5.71 (s, 1H), 3.58-3.46 (m, 2H), 1.77-1.63 (m, 2H), 1.46 (dq, J=14.6, 7.3 Hz, 2H), 1.00 (t, J=7.3 Hz, 3H). LC-MS m/z: 235.3 [M+H]⁺. HPLC Purity (214 nm): 88%; t_{R}=8.59 min.

### Example 68—N-Butyl-4-fluoro-1H-benzo[d]imidazole-1-carboxamide

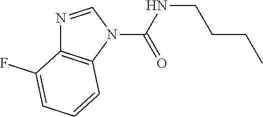 [SMILES: CCCCNC(=O)n1cnc2c(F)cccc21]

Following general procedure B, 4-fluoro-1H-benzo[d]imidazole (136 mg, 1.0 mmol) and 1-isocyanatobutane (99 mg, 1.0 mmol) afforded the title compound (50 mg, 21.3%) as a white solid. ¹H NMR (400 MHz, CDCl₃) δ 8.39 (s, 1H), 7.65 (d, J=8.3 Hz, 1H), 7.34 (td, J=8.1, 4.7 Hz, 1H), 7.08 (dd, J=10.0, 8.2 Hz, 1H), 5.97 (s, 1H), 3.53 (dd, J=13.0, 7.1 Hz, 2H), 1.69 (dt, J=14.9, 7.4 Hz, 2H), 1.52-1.34 (m, 2H), 1.00 (t, J=7.3 Hz, 3H). LC-MS m/z: 236.2 [M+H]⁺. HPLC Purity (214 nm): 100%; t_{R}=7.43 min.

### Example 69—N-Butyl-6-fluoro-1H-benzo[d]imidazole-1-carboxamide

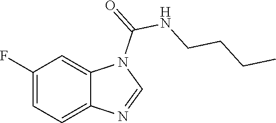 [SMILES: CCCCNC(=O)n1cnc2ccc(F)cc21]

Following general procedure B, 5-fluoro-1H-benzo[d]imidazole (200 mg, 1.5 mmol) and 1-isocyanatobutane (219 mg, 2.2 mmol) afforded the title compound (7.4 mg, 2.3%) as a colorless oil. ¹H NMR (400 MHz, CDCl₃) δ 8.35 (s, 1H), 7.73 (dd, J=8.9, 4.9 Hz, 1H), 7.64 (dd, J=8.9, 2.4 Hz, 1H), 7.11 (td, J=9.1, 2.5 Hz, 1H), 5.86 (s, 1H), 3.52 (dd, J=13.0, 7.1 Hz, 2H), 1.69 (dt, J=14.9, 7.5 Hz, 2H), 1.46 (dq, J=14.6, 7.3 Hz, 2H), 0.99 (t, J=7.3 Hz, 3H). LC-MS m/z: 235.3 [M+H]⁺. HPLC Purity (214 nm): 95.80%; t_{R}=8.19 min.

### Examples 70a and 70b—N-Butyl-6-(trifluoromethoxy)-1H-benzo[d]imidazole-1-carboxamide and N-butyl-5-(trifluoromethoxy)-1H-benzo[d]imidazole-1-carboxamide

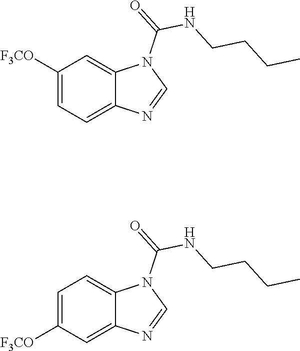 [SMILES: CCCCNC(=O)n1cnc2ccc(OC(F)(F)F)cc21 ; CCCCNC(=O)n1cnc2cc(OC(F)(F)F)ccc21]

Following general procedure F, 2-nitro-5-(trifluoromethoxy)aniline (1.0 g, 4.5 mmol) afforded 4-(trifluoromethoxy)benzene-1,2-diamine (750 mg, 75%). LC-MS m/z: 193.2 [M+H]⁺. Purity (214 nm): 100%; t_{R}=0.75 min.

Following general procedure G (method B), 4-(trifluoromethoxy)benzene-1,2-diamine (750 mg, 3.9 mmol) afforded 5-(trifluoromethoxy)-1H-benzo[d]imidazole (800 mg, 99%). LC-MS m/z: 203.2 [M+H]⁺. HPLC purity (214 nm): 100%; t_{R}=0.69 min.

Following general procedure C, 5-(trifluoromethoxy)-1H-benzo[d]imidazole (400 mg, 2.0 mmol) and 1-butylamine (140 mg, 2.0 mmol) afforded N-butyl-6-(trifluoromethoxy)-1H-benzo[d]imidazole-1-carboxamide (30 mg, 5.0%) as a pink solid and N-butyl-5-(trifluoromethoxy)-1H-benzo[d]imidazole-1-carboxamide (30 mg, 5.0%) as a white solid.

N-butyl-6-(trifluoromethoxy)-1H-benzo[d]imidazole-1-carboxamide: ¹H NMR (400 MHz, CDCl₃) δ 8.37 (s, 1H), 7.86 (d, J=1.1 Hz, 1H), 7.81 (d, J=8.8 Hz, 1H), 7.25 (d, J=1.3 Hz, 1H), 5.73 (s, 1H), 3.53 (td, J=7.2, 5.8 Hz, 2H), 1.74-1.67 (m, 2H), 1.47 (dq, J=14.6, 7.3 Hz, 2H), 1.00 (t, J=7.4 Hz, 3H). LC-MS m/z: 302.3 [M+H]⁺. HPLC Purity (214 nm): >99%; t_{R}=9.76 min.

N-butyl-5-(trifluoromethoxy)-1H-benzo[d]imidazole-1-carboxamide: ¹H NMR (400 MHz, CDCl₃) δ 8.41 (s, 1H), 7.91 (d, J=8.9 Hz, 1H), 7.69 (s, 1H), 7.30 (dd, J=8.9, 1.5 Hz, 1H), 5.74 (s, 1H), 3.53 (td, J=7.2, 5.8 Hz, 2H), 1.78-1.64 (m, 2H), 1.47 (dq, J=14.6, 7.3 Hz, 2H), 1.00 (t, J=7.4 Hz, 3H). LC-MS m/z: 302.3[M+H]⁺. HPLC Purity (214 nm): >99%; t_{R}=9.66 min.

### Example 71—4-Methoxy-N-(2-phenoxyethyl)-1H-benzo[d]imidazole-1-carboxamide

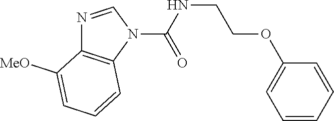 [SMILES: COc1cccc2c1ncn2C(=O)NCCOc1ccccc1]

Following general procedure C, 4-methoxy-1H-benzo[d]imidazole (180 mg, 1.21 mmol) and 2-phenoxyethanamine (333.3 mg, 2.42 mmol) afforded the title compound (127.4 mg, 33.6%) as a white solid. ¹H NMR (400 MHz, CDCl₃) δ 8.36 (s, 1H), 7.40-7.30 (m, 4H), 7.03-6.94 (m, 3H), 6.81 (d, J=8.0 Hz, 1H), 6.30 (s, 1H), 4.23 (t, J=5.6 Hz, 2H), 4.05 (s, 3H), 3.96-3.92 (m, 2H). LC-MS m/z: 312.1 [M+H]⁺. HPLC Purity (214 nm): 96.39%; t_{R}=8.12 min.

### Example 72—N-(2-Phenoxyethyl)-4-(trifluoromethyl)-1H-benzo[d]imidazole-1-carboxamide

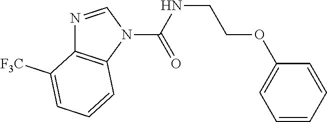 [SMILES: O=C(NCCOc1ccccc1)n1cnc2c(C(F)(F)F)cccc21]

Following general procedure F, 2-nitro-3-(trifluoromethyl)aniline (600 mg, 2.9 mmol) afforded 3-(trifluoromethyl)benzene-1,2-diamine (430 mg, 84%) as a yellow solid. LC-MS m/z: 177.1 [M+H]⁺. HPLC Purity (214 nm): 89.75%; t_{R}=1.70 min.

Following general procedure G (method A), 3-(trifluoromethyl) benzene-1,2-diamine (430 mg, 2.4 mmol) afforded 4-(trifluoromethyl)-1H-benzo[d]imidazole (360 mg, 80%). LC-MS m/z: 187.3 [M+H]⁺. Purity (214 nm): 93%; t_{R}=0.72 min.

Following general procedure C, 4-(trifluoromethyl)-1H-benzo[d]imidazole (360 mg, 1.9 mmol) and 2-phenoxyethanamine (123 mg, 0.9 mmol) afforded the title compound (51 mg, 7.7%) as a white solid. ¹H NMR (400 MHz, CDCl₃) δ 8.48 (s, 1H), 8.15 (d, J=8.3 Hz, 1H), 7.65 (d, J=7.6 Hz, 1H), 7.47 (t, J=8.0 Hz, 1H), 7.34-7.28 (m, 2H), 7.00 (t, J=7.4 Hz, 1H), 6.95-6.87 (m, 2H), 6.46 (s, 1H), 4.27-4.20 (m, 2H), 3.94 (dd, J=10.2, 5.4 Hz, 2H). LC-MS m/z: 350.1[M+H]⁺. HPLC Purity (214 nm): 100%; t_{R}=9.16 min.

### Example 73—4-Fluoro-N-(2-phenoxyethyl)-1H-benzo[d]imidazole-1-carboxamide

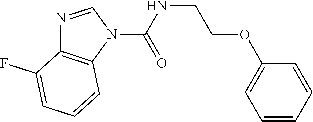 [SMILES: O=C(NCCOc1ccccc1)n1cnc2c(F)cccc21]

Following general procedure C, 4-fluoro-1H-benzo[d]imidazole (136 mg, 1.0 mmol) and 2-phenoxyethanamine (137 mg, 1.0 mmol) afforded the title compound (40 mg, 13.3%) as a yellow solid. ¹H NMR (400 MHz, CDCl₃) δ 8.41 (s, 1H), 7.66 (d, J=8.3 Hz, 1H), 7.38-7.28 (m, 3H), 7.10-7.03 (m, 1H), 7.00 (t, J=7.4 Hz, 1H), 6.92 (d, J=8.0 Hz, 2H), 6.59 (s, 1H), 4.24 (t, J=5.0 Hz, 2H), 3.94 (dd, J=10.3, 5.3 Hz, 2H). LC-MS m/z: 300 [M+H]⁺. HPLC Purity (214 nm): 95.23%; t_{R}=1.98 min.

### Example 74—4-Cyano-N-(2-phenoxyethyl)-1H-benzo[d]imidazole-1-carboxamide

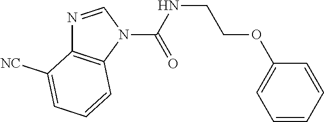 [SMILES: N#Cc1cccc2c1ncn2C(=O)NCCOc1ccccc1]

Following general procedure C, 1H-benzo[d]imidazole-4-carbonitrile (100 mg, 0.70 mmol) and 2-phenoxyethanamine (115 mg, 0.84 mmol) afforded the title compound (38 mg, 17%) as a white solid. ¹H NMR (400 MHz, CDCl₃) δ 8.48 (s, 1H), 8.21 (d, J=8.4 Hz, 1H), 7.70 (d, J=7.2 Hz, 1H), 7.48 (t, J=8.0 Hz, 1H), 7.32 (t, J=8.4 Hz, 2H), 7.01 (t, J=7.2 Hz, 1H), 6.93 (d, J=8.0 Hz, 2H), 6.29 (t, J=5.6 Hz, 1H), 4.25 (t, J=4.8 Hz, 2H), 3.95 (q, J=4.8 Hz, 2H). LC-MS m/z: 307.1 [M+H]⁺. HPLC Purity (214 nm): 98%; t_{R}=8.22 min.

### Example 75—N-isoButyl-4-methoxy-5-(pyrimidin-5-yl)-1H-benzo[d]imidazole-1-carboxamide

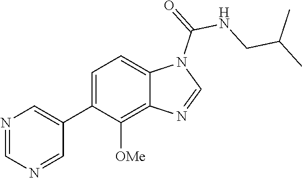 [SMILES: COc1c(-c2cncnc2)ccc2c1ncn2C(=O)NCC(C)C]

Following general procedure A, 6-bromo-7-methoxy-1H-benzo[d]imidazole (552 mg, 2.0 mmol) and pyrimidin-5-ylboronic acid (369 mg, 3.0 mmol) afforded 4-methoxy-5-(pyrimidin-5-yl)-1H-benzo[d]imidazole (230 mg, crude) as a yellow solid. LC-MS m/z: 227.2 [M+H]⁺. HPLC purity (214 nm): 82%; t_{R}=0.58 min.

Following general procedure D, 4-methoxy-5-(pyrimidin-5-yl)-1H-benzo[d]imidazole (226 mg, 1.0 mmol) and 2-methylpropan-1-amine (73 mg, 1 mmol) afforded the title compound (26.2 mg, 8.1%) as a white solid. ¹H NMR (400 MHz, CDCl₃) δ 9.18 (s, 1H), 8.94 (s, 2H), 8.38 (s, 1H), 7.55 (t, J=11.7 Hz, 1H), 7.35 (d, J=8.4 Hz, 1H), 5.85 (s, 1H), 4.36 (s, 3H), 3.57-3.21 (m, 2H), 2.02 (dp, J=13.4, 6.7 Hz, 1H), 1.06 (d, J=6.7 Hz, 6H). LC-MS m/z: 326.0 [M+H]⁺. HPLC Purity (214 nm): 100%; t_{R}=7.85 min.

### Example 76—N-iso-Butyl-5-(pyrimidin-5-yl)-4-(trifluoromethyl)-1H-benzo[d]imidazole-1-carboxamide

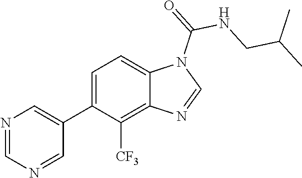 [SMILES: CC(C)CNC(=O)n1cnc2c(C(F)(F)F)c(-c3cncnc3)ccc21]

N-(3-(trifluoromethyl)phenyl)acetamide (5.0 g, 24.6 mmol) was added in small portions to cold fuming nitric acid (20 mL) in a dry round bottom flask at −10° C. The reaction mixture was stirred for another 10 min, then quenched with water (100 mL) and extracted with EA (100 mL×2). The combined organic layers were dried over Na₂SO₄, concentrated and purified by silica gel column chromatography (20% EA/hexane) to afford N-(2-nitro-3-(trifluoromethyl)phenyl)acetamide as a yellow solid (1.2 g, 19.6%). LC-MS m/z: 248.9 [M+H]⁺. HPLC Purity (214 nm): 92%; t_{R}=1.79 min.

To a solution of N-(2-nitro-3-(trifluoromethyl)phenyl)acetamide (1.2 g, 4.8 mmol) in EtOH (12 mL) was added 20% aq. NaOH (6.0 mL) and the mixture was heated to reflux for 2 h. The reaction mixture was then cooled to RT and concentrated, diluted with water (50 mL) and extracted with EA (50 mL×2). The combined organic layers were dried over Na₂SO₄ and concentrated to dryness to give 2-nitro-3-(trifluoromethyl)aniline as a yellow solid (0.95 g, 98%). LC-MS m/z: 207.0 [M+H]⁺. HPLC Purity (214 nm): 92%; t_{R}=1.93 min.

A solution of 2-nitro-3-(trifluoromethyl)aniline (650 mg, 3.16 mmol) and NBS (561 mg, 3.16 mmol) in MeCN (10 mL) was stirred at RT for 16 h and the reaction mixture was then poured into water (30 mL) and extracted with EA (50 mL×2). The combined organic layers were washed with brine (50 mL×1), dried over Na₂SO₄, filtered and concentrated to give a residue which was purified by silica gel column chromatography (PE:EA=3:1) to give 4-bromo-2-nitro-3-(trifluoromethyl)aniline (700 mg, 78%) as a yellow solid. LC-MS m/z: 284.1 [M+H]⁺. HPLC Purity (214 nm): 85%; t_{R}=2.03 min.

Following general procedure A, 4-bromo-2-nitro-3-(trifluoromethyl)aniline (800 mg, 2.82 mmol) and pyrimidin-5-ylboronic acid (420 g, 3.38 mmol) afforded 2-nitro-4-(pyrimidin-5-yl)-3-(trifluoromethyl)aniline (580 mg, 72%) as a brown oil. LC-MS m/z: 285.0 [M+H]⁺. HPLC Purity (214 nm): 99%; t_{R}=1.78 min.

Following general procedure F, 2-nitro-4-(pyrimidin-5-yl)-3-(trifluoromethyl)aniline (600 mg, 2.11 mmol) afforded 4-(pyrimidin-5-yl)-3-(trifluoromethyl)benzene-1,2-diamine (536 mg, 99%) as a brown solid. LC-MS m/z: 255.0 [M+H]⁺. HPLC Purity (214 nm): 99%; t_{R}=1.58 min.

Following general procedure G (method B), 4-(pyrimidin-5-yl)-3-(trifluoromethyl)benzene-1,2-diamine (540 mg, 2.13 mmol) afforded 5-(pyrimidin-5-yl)-4-(trifluoromethyl)-1H-benzo[d]imidazole (300 mg, 53.3%) as a yellow solid. LC-MS m/z: 265.0 [M+H]⁺. HPLC Purity (214 nm): 99%; t_{R}=1.46 min.

Following general procedure C, 5-(pyrimidin-5-yl)-4-(trifluoromethyl)-1H-benzo[d]imidazole (140 mg, 0.53 mmol) and 2-methylpropan-1-amine (46 mg, 0.64 mmol) afforded the title compound (103.3 mg, 53.6%) as a yellow solid. ¹H NMR (400 MHz, CDCl₃) δ 9.27 (s, 1H), 8.76 (s, 2H), 8.51 (s, 1H), 8.29 (d, J=8.4 Hz, 1H), 7.32 (d, J=8.4 Hz, 1H), 5.95 (brs, 1H), 3.39 (t, J=6.4 Hz, 2H), 2.06-1.99 (m, 1H), 1.05 (d, J=6.8 Hz, 6H). LC-MS m/z: 364.2 [M+H]⁺. HPLC Purity (214 nm): 95%; t_{R}=8.05 min.

### Examples 77a and 77b—N-iso-Butyl-5-(pyrimidin-5-yl)-6-(trifluoromethyl)-1H-benzo[d]imidazole-1-carboxamide and N-iso-butyl-6-(pyrimidin-5-yl)-5-(trifluoromethyl)-1H-benzo[d]imidazole-1-carboxamide

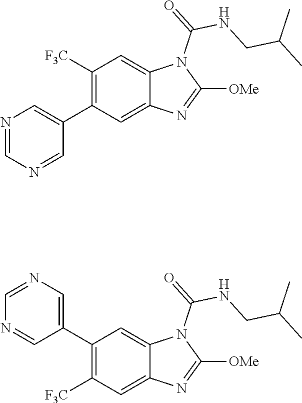 [SMILES: COc1nc2cc(-c3cncnc3)c(C(F)(F)F)cc2n1C(=O)NCC(C)C ; COc1nc2cc(C(F)(F)F)c(-c3cncnc3)cc2n1C(=O)NCC(C)C]

Following general procedure A, 5-chloro-2-nitro-4-(trifluoromethyl)aniline (1.0 g, 4.16 mmol) and pyrimidin-5-ylboronic acid (1.3 g, 10.39 mmol) afforded 2-nitro-5-(pyrimidin-5-yl)-4-(trifluoro-methyl)aniline (305 mg, 26%) as a light brown solid. LC-MS m/z: 284.9 [M+H]⁺. HPLC Purity (214 nm): 86.9%; t_{R}=1.88 min.

Following general procedure F, 2-nitro-5-(pyrimidin-5-yl)-4-(trifluoromethyl)aniline (320 mg, 1.13 mmol) afforded 4-(pyrimidin-5-yl)-5-(trifluoromethyl)benzene-1,2-diamine (240 mg, 83.9%) as a brown semisolid. LC-MS m/z: 255.0 [M+H]⁺. HPLC Purity (214 nm): 94.5%; t_{R}=1.67 min.

Following general procedure G (method A), 4-(pyrimidin-5-yl)-5-(trifluoromethyl) benzene-1,2-diamine (240 mg, 0.94 mmol) afforded 5-(pyrimidin-5-yl)-6-(trifluoromethyl)-1H-benzo[d]imidazole (290 mg, 95%) as a light yellow semisolid. LC-MS m/z: 265.0 [M+H]⁺. HPLC Purity (214 nm): 97.4%; t_{R}=1.69 min.

Following general procedure C, 5-(pyrimidin-5-yl)-6-(trifluoromethyl)-1H-benzo[d]imidazole (290 mg, 1.1 mmol) and 2-methylpropan-1-amine (80 mg, 1.1 mmol) afforded N-iso-butyl-5-(pyrimidin-5-yl)-6-(trifluoromethyl)-1H-benzo[d]imidazole-1-carboxamide (20.7 mg, 3.2%) as a white solid and N-iso-butyl-6-(pyrimidin-5-yl)-5-(trifluoromethyl)-1H-benzo[d]imidazole-1-carboxamide (51.1 mg, 7.9%) as a white solid.

N-iso-butyl-5-(pyrimidin-5-yl)-6-(trifluoromethyl)-1H-benzo[d]imidazole-1-carboxamide: ¹H NMR (400 MHz, CDCl₃) δ 9.28 (s, 1H), 8.77 (s, 2H), 8.50 (s, 1H), 8.51 (s, 1H), 7.77 (s, 1H), 5.90 (s, 1H), 3.42 (dd, J=6.4, 6.0 Hz, 2H), 2.09-2.00 (m, 1H), 1.09 (d, J=6.7 Hz, 6H). LC-MS m/z: 364.1 [M+H]⁺. HPLC Purity (214 nm): 82%; t_{R}=8.33 min.

N-iso-butyl-6-(pyrimidin-5-yl)-5-(trifluoromethyl)-1H-benzo[d]imidazole-1-carboxamide: ¹H NMR (400 MHz, CDCl₃) δ 9.28 (s, 1H), 8.77 (s, 2H), 8.52 (s, 1H), 8.30 (s, 1H), 8.03 (s, 1H), 6.07 (brs, 1H), 3.37 (dd, J=6.8, 6.0 Hz, 2H), 2.03-1.98 (m, 1H), 1.03 (d, J=6.7 Hz, 6H). LC-MS m/z: 364.0 [M+H]⁺. HPLC Purity (214 nm): 96%; t_{R}=8.11 min.

### Example 78—N-(2-(4-Methylpiperazin-1-yl)ethyl)-5-(trifluoromethyl)-1H-benzo[d]imidazole-1-carboxamide

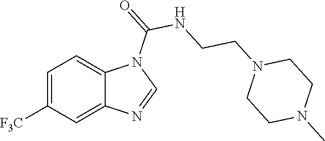 [SMILES: CN1CCN(CCNC(=O)n2cnc3cc(C(F)(F)F)ccc32)CC1]

Following general procedure C, 5-(trifluoromethyl)-1H-benzo[d]imidazole (200 mg, 1.07 mmol) and 2-(4-methylpiperazin-1-yl)ethanamine (307.8 mg, 2.15 mmol) afforded the title compound (265 mg, 22.7%) as a white solid. ¹H NMR (400 MHz, CDCl₃) δ 8.54 (s, 1H), 8.13 (s, 1H), 8.04 (d, J=8.8 Hz, 1H), 7.66 (dd, J=8.4, 0.8 Hz, 1H), 6.82 (brs, 1H), 3.64-3.60 (m, 2H), 2.71 (t, J=6.0 Hz, 2H), 2.62-2.47 (m, 8H), 2.33 (s, 3H). LC-MS m/z: 356.2 [M+H]⁺. HPLC Purity (214 nm): 98.86%; t_{R}=5.31 min.

### Examples 79a and 79b—N-iso-Butyl-6-methoxy-5-(pyrimidin-5-yl)-1H-benzo[d]imidazole-1-carboxamide and N-iso-butyl-5-methoxy-6-(pyrimidin-5-yl)-1H-benzo[d]imidazole-1-carboxamide

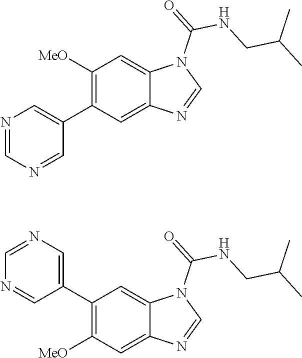 [SMILES: COc1cc2c(cc1-c1cncnc1)ncn2C(=O)NCC(C)C ; COc1cc2ncn(C(=O)NCC(C)C)c2cc1-c1cncnc1]

Following general procedure A, 5-bromo-6-methoxy-1H-benzo[d]imidazole (366 mg, 1.61 mmol) and pyrimidin-5-ylboronic acid (409 mg, 3.30 mmol) afforded 6-methoxy-5-(pyrimidin-5-yl)-1H-benzo[d]imidazole (230 mg, 63%) as a brown solid. LC-MS m/z: 227.3 [M+H]⁺. Purity (254 nm): >95%; t_{R}=0.49 min.

Following general procedure D, 6-methoxy-5-(pyrimidin-5-yl)-1H-benzo[d]imidazole (200 mg, 0.885 mmol) and 2-methylpropan-1-amine (74 mg 1.014 mmol) afforded N-iso-butyl-6-methoxy-5-(pyrimidin-5-yl)-1H-benzo[d]imidazole-1-carboxamide (10.1 mg, 3.5%) and N-iso-butyl-5-methoxy-6-(pyrimidin-5-yl)-1H-benzo[d]imidazole-1-carboxamide (10.3 mg, 3.6%) as white solids.

N-iso-butyl-6-methoxy-5-(pyrimidin-5-yl)-1H-benzo[d]imidazole-1-carboxamide: ¹H NMR (500 MHz, CDCl₃) δ 9.19 (s, 1H), 8.94 (s, 2H), 8.36 (s, 1H), 7.88 (s, 1H), 7.41 (s, 1H), 5.78 (s, 1H), 3.90-3.88 (m, 3H), 3.38-3.32 (m, 2H), 1.99 (dt, J=13.5, 6.7 Hz, 1H), 1.03 (d, J=6.7 Hz, 6H). LC-MS m/z: 326.0 [M+H]⁺. HPLC Purity (214 nm): >97%; t_{R}=1.77 min.

N-iso-butyl-5-methoxy-6-(pyrimidin-5-yl)-1H-benzo[d]imidazole-1-carboxamide: ¹H NMR (500 MHz, CDCl₃) δ 9.19 (s, 1H), 8.94 (s, 2H), 8.36 (s, 1H), 8.36 (s, 1H), 7.88 (s, 1H), 7.41 (s, 1H), 5.78 (s, 1H), 3.90 (s, 3H), 3.38-3.32 (m, 2H), 1.99 (dt, J=13.5, 6.7 Hz, 1H), 1.03 (d, J=6.7 Hz, 6H). LC-MS m/z: 326.0 [M+H]⁺. HPLC Purity (214 nm): >89%; t_{R}=1.82 min.

### Example 80—N-(4,4,4-Trifluorobutyl)-1H-benzo[d]imidazole-1-carboxamide

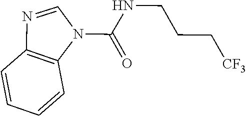 [SMILES: O=C(NCCCC(F)(F)F)n1cnc2ccccc21]

Following general procedure C, 1H-benzo[d]imidazole (150 mg, 1.27 mmol) and 4,4,4-trifluorobutan-1-amine (177 mg, 1.40 mmol) afforded the title compound (130.6 mg, 37.8%) as a white solid. ¹H NMR (400 MHz, CDCl₃) δ 8.34 (s, 1H), 7.88 (d, J=7.6 Hz, 1H), 7.76 (dd, J=8.0, 0.8 Hz, 1H), 7.43-7.35 (m, 2H), 6.77 (s, 1H), 3.59 (q, J=6.8 Hz, 2H), 2.29-2.17 (m, 2H), 2.08-1.95 (m, 2H). LC-MS m/z: 272.0 [M+H]⁺. HPLC Purity (214 nm): 98%; t_{R}=7.86 min.

### Examples 81a and 81b—N-iso-Butyl-2-methoxy-6-(pyrimidin-5-yl)-1H-benzo[d]imidazole-1-carboxamide and N-iso-butyl-2-methoxy-5-(pyrimidin-5-yl)-1H-benzo[d]imidazole-1-carboxamide

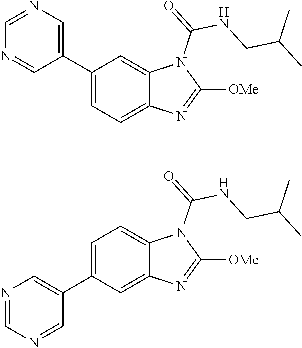 [SMILES: COc1nc2ccc(-c3cncnc3)cc2n1C(=O)NCC(C)C ; COc1nc2cc(-c3cncnc3)ccc2n1C(=O)NCC(C)C]

Following general procedure C, 2-methoxy-6-(pyrimidin-5-yl)-1H-benzo[d]imidazole (200 mg, 0.88 mmol) and 2-methylpropan-1-amine (77 mg, 1.06 mmol) afforded N-iso-butyl-2-methoxy-6-(pyrimidin-5-yl)-1H-benzo[d]imidazole-1-carboxamide (30 mg, 11%) and N-iso-butyl-2-methoxy-5-(pyrimidin-5-yl)-1H-benzo[d]imidazole-1-carboxamide (28 mg, 10%) as white solids.

N-iso-butyl-2-methoxy-6-(pyrimidin-5-yl)-1H-benzo[d]imidazole-1-carboxamide: ¹H NMR (400 MHz, CDCl₃) δ 9.19 (s, 1H), 9.00 (s, 2H), 8.31 (d, J=8.4 Hz, 1H), 7.73 (s, 1H), 7.45 (d, J=8.8 Hz, 1H), 7.03 (t, J=5.2 Hz, 1H), 4.38 (s, 3H), 3.31 (t, J=6.4 Hz, 2H), 1.99-1.92 (m, 1H), 1.02 (d, J=6.8 Hz, 6H). LC-MS m/z: 326.1 [M+H]⁺. HPLC Purity (214 nm): 96%; t_{R}=7.67 min.

N-iso-butyl-2-methoxy-5-(pyrimidin-5-yl)-1H-benzo[d]imidazole-1-carboxamide: ¹H NMR (400 MHz, CDCl₃) δ 9.18 (s, 1H), 9.00 (s, 2H), 8.47 (s, 1H), 7.63 (d, J=6.4 Hz, 1H), 7.41 (d, J=8.4 Hz, 1H), 7.05 (t, J=5.2 Hz, 1H), 4.39 (s, 3H), 3.31 (t, J=6.4 Hz, 2H), 1.99-1.92 (m, 1H), 1.02 (d, J=6.4 Hz, 6H). LC-MS m/z: 326.1 [M+H]⁺. HPLC Purity (214 nm): 95%; t_{R}=7.78 min.

### Examples 82a and 82b—N-Butyl-2-methoxy-6-(pyrimidin-5-yl)-1H-benzo[d]imidazole-1-carboxamide and N-Butyl-2-methoxy-5-(pyrimidin-5-yl)-1H-benzo[d]imidazole-1-carboxamide

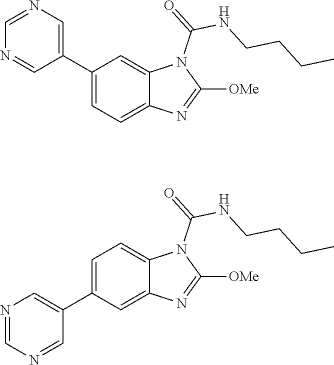 [SMILES: CCCCNC(=O)n1c(OC)nc2ccc(-c3cncnc3)cc21 ; CCCCNC(=O)n1c(OC)nc2cc(-c3cncnc3)ccc21]

Following general procedure B, 2-methoxy-6-(pyrimidin-5-yl)-1H-benzo[d]imidazole (150 mg, 0.66 mmol) and (2-isocyanatoethyl)benzene (326 mg, 3.30 mmol) afforded N-butyl-2-methoxy-6-(pyrimidin-5-yl)-1H-benzo[d]imidazole-1-carboxamide (43.9 mg, 20.3%) and N-butyl-2-methoxy-5-(pyrimidin-5-yl)-1H-benzo[d]imidazole-1-carboxamide (25 mg, 11.7%) both as white solids.

N-butyl-2-methoxy-6-(pyrimidin-5-yl)-1H-benzo[d]imidazole-1-carboxamide: ¹H NMR (400 MHz, CDCl₃) δ 9.19 (s, 1H), 8.99 (s, 2H), 8.31 (d, J=8.4 Hz, 1H), 7.72 (d, J=1.6 Hz, 1H), 7.45 (dd, J=8.8, 1.6 Hz, 1H), 6.97 (t, J=4.4 Hz, 1H), 4.37 (s, 3H), 3.47 (q, J=6.8 Hz, 2H), 1.70-1.62 (m, 2H), 1.50-1.40 (m, 2H), 1.00 (t, J=7.6 Hz, 3H). LC-MS m/z: 326.1 [M+H]⁺. HPLC Purity (214 nm): 99%; t_{R}=8.00 min.

N-butyl-2-methoxy-5-(pyrimidin-5-yl)-1H-benzo[d]imidazole-1-carboxamide: ¹H NMR (400 MHz, CDCl₃) δ 9.18 (s, 1H), 9.00 (s, 2H), 8.47 (d, J=1.6 Hz, 1H), 8.63 (d, J=8.0 Hz, 1H), 7.47 (dd, J=8.0, 1.6 Hz, 1H), 6.99 (t, J=6.0 Hz, 1H), 4.38 (s, 3H), 3.47 (q, J=7.2 Hz, 2H), 1.70-1.62 (m, 2H), 1.49-1.40 (m, 2H), 1.00 (t, J=7.6 Hz, 3H). LC-MS m/z: 326.1 [M+H]⁺. HPLC Purity (214 nm): 97%; t_{R}=7.87 min.

### Example 83—N-iso-Butyl-2-methoxy-6-(pyridin-3-yl)-3H-imidazo[4,5-b]pyridine-3-carboxamide

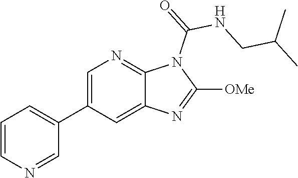 [SMILES: COc1nc2cc(-c3cccnc3)cnc2n1C(=O)NCC(C)C]

Following general procedure G (method A), 5-bromopyridine-2,3-diamine (7.22 g, 38.4 mmol) afforded 6-bromo-2-methoxy-1H-imidazo[4,5-b]pyridine (2.5 g, 28.6%) as a yellow solid. LC-MS m/z: 228.1 [M+H]⁺. HPLC Purity (254 nm): 61%; t_{R}=1.20 min.

Following general procedure A, 6-bromo-2-methoxy-1H-imidazo[4,5-b]pyridine (2.5 g, 11.0 mmol) and pyridine-3-ylboronic acid (1.6 g, 13.2 mmol) afforded 2-methoxy-6-(pyridin-3-yl)-1H-imidazo[4,5-b]pyridine (0.8 g, 32%) as an off-white solid. LC-MS m/z: 227.1 [M+H]⁺. HPLC Purity (254 nm): 77%; t_{R}=1.20 min.

Following general procedure B, 2-methoxy-6-(pyridin-3-yl)-1H-imidazo[4,5-b]pyridine (40 mg, 0.18 mmol) and 1-isocyanato-2-methylpropane (87.64 mg, 0.88 mmol) afforded N-isobutyl-2-methoxy-6-(pyridin-3-yl)-3H-imidazo[4,5-b]pyridine-3-carboxamide (4.1 mg, 7.1%) as a white solid. ¹H NMR (400 MHz, CDCl₃) δ 9.47 (brs, 1H), 8.88 (d, J=1.6 Hz, 1H), 8.67 (dd, J=4.8 Hz, 1.6 Hz, 1H), 8.38 (d, J=2 Hz, 1H), 8.00 (d, J=2 Hz, 1H), 7.90 (dt, J=8.4 Hz, 2 Hz, 1H), 7.45-7.42 (m, 1H), 4.33 (s, 3H), 3.35 (dd, J=7.0, 6.0 Hz, 2H), 2.04-1.97 (m, 1H), 1.05 (d, J=6.8 Hz, 6H). LC-MS m/z: 326.0 [M+H]⁺. HPLC Purity (214 nm): 97.73%; t_{R}=7.24 min.

### Example 84—N-Butyl-3-methyl-2-oxo-5-(pyrimidin-5-yl)-2,3-dihydro-1H-imidazo[4,5-b]pyridine-1-carboxamide

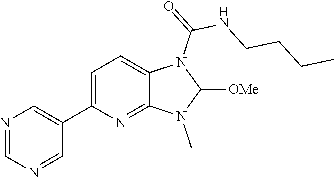 [SMILES: CCCCNC(=O)N1c2ccc(-c3cncnc3)nc2N(C)C1OC]

Following general procedure A, 6-bromo-3-nitropyridin-2-amine (1.5 g, 6.91 mmol) and pyrimidin-5-ylboronic acid (1.02 g, 8.29 mmol) afforded 3-nitro-6-(pyrimidin-5-yl)pyridin-2-amine (1.3 g, 86.6%) as a yellow solid. LC-MS m/z: 216.95. [M+H]⁺. HPLC Purity (254 nm): 97.78%; t_{R}=1.59 min.

Following general procedure F, 6-bromo-3-nitropyridin-2-amine (1.3 g, 5.9 mmol) afforded 6-(pyrimidin-5-yl)pyridine-2,3-diamine (1.0 g, 89%) as a grey solid. LC-MS m/z: 187.09. [M+H]⁺. HPLC Purity (214 nm): 97.75%; t_{R}=1.32 min.

Following general procedure G (method A), 6-(pyrimidin-5-yl)pyridine-2,3-diamine (300 mg, 0.16 mmol) afforded 2-methoxy-5-(pyrimidin-5-yl)-3H-imidazo[4,5-b]pyridine (100 mg, 41%) as a yellow oil. LC-MS m/z: 227.08. [M+H]⁺. HPLC Purity (214 nm): 81.86%; t_{R}=1.40 min.

Following general procedure B, 2-methoxy-5-(pyrimidin-5-yl)-3H-imidazo[4,5-b]pyridine (200 mg, 0.88 mmol) and 1-isocyanatobutane (130.7 mg, 1.32 mmol) afforded the title compound (20 mg, 6.9%) as a white solid. ¹H NMR (400 MHz, CDCl₃) δ 9.35 (s, 2H), 9.24 (s, 1H), 8.53 (s, 1H), 8.45 (d, J=8.2 Hz, 1H), 7.60 (d, J=8.2 Hz, 1H), 3.59 (s, 3H), 3.45 (dt, J=12.8, 6.8 Hz, 2H), 1.68-1.61 (m, 2H), 1.53-1.36 (m, 2H), 0.98 (t, J=7.3 Hz, 3H). LC-MS m/z: 326.15. [M+H]⁺. HPLC Purity (214 nm): 100%; t_{R}=8.76 min.

### Examples 85a and 85b—N-Butyl-2-methoxy-5-(pyrimidin-5-yl)-3H-imidazo[4,5-b]pyridine-3-carboxamide and N-butyl-2-methoxy-5-(pyrimidin-5-yl)-1H-imidazo[4,5-b]pyridine-1-carboxamide

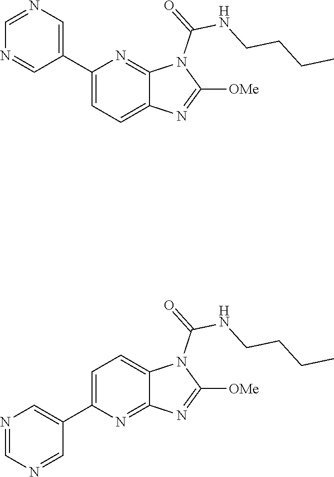 [SMILES: CCCCNC(=O)n1c(OC)nc2ccc(-c3cncnc3)nc21 ; CCCCNC(=O)n1c(OC)nc2nc(-c3cncnc3)ccc21]

Following general procedure B, 2-methoxy-5-(pyrimidin-5-yl)-3H-imidazo[4,5-b]pyridine (160 mg, 0.71 mmol) and 1-isocyanatobutane (100 mg, 1.05 mmol) afforded N-butyl-2-methoxy-5-(pyrimidin-5-yl)-3H-imidazo[4,5-b]pyridine-3-carboxamide (7.0 mg, 3.0%), N-butyl-3-methyl-2-oxo-5-(pyrimidin-5-yl)-2,3-dihydro-1H-imidazo[4,5-b]pyridine-1-carboxamide (2.2 mg, 1.0%) and N-butyl-2-methoxy-5-(pyrimidin-5-yl)-1H-imidazo[4,5-b]pyridine-1-carboxamide (27.3 mg, 11.9%) all as white solids.

N-butyl-2-methoxy-5-(pyrimidin-5-yl)-3H-imidazo[4,5-b]pyridine-3-carboxamide ¹H NMR (400 MHz, CDCl₃) δ 9.27 (s, 1H), 9.26 (d, J=6.9 Hz, 3H), 7.95 (d, J=8.2 Hz, 1H), 7.72 (d, J=8.2 Hz, 1H), 4.33 (s, 3H), 3.53 (dd, J=12.5, 7.0 Hz, 2H), 1.78-1.60 (m, 2H), 1.47 (s, 2H), 0.99 (t, J=7.4 Hz, 3H). LC-MS m/z: 326.15. [M+H]⁺. HPLC Purity (214 nm): 100%; t_{R}=7.46 min.

N-butyl-2-methoxy-5-(pyrimidin-5-yl)-1H-imidazo[4,5-b]pyridine-1-carboxamide ¹H NMR (400 MHz, CDCl₃) δ 9.43 (s, 2H), 9.23 (s, 1H), 8.52 (d, J=8.3 Hz, 1H), 7.63 (t, J=34.2 Hz, 1H), 6.97 (d, J=19.7 Hz, 1H), 4.46 (s, 3H), 3.48 (dd, J=12.9, 7.1 Hz, 3H), 1.69-1.65 (m, 2H), 1.43 (d, J=7.2 Hz, 2H), 1.00 (t, J=7.4 Hz, 3H). LC-MS m/z: 326.15. [M+H]⁺. HPLC Purity (214 nm): 100%; t_{R}=7.58 min.

### Example 86—4-((1-Methylpiperidin-4-yl)oxy)-N-phenethyl-1H-benzo[d]imidazole-1-carboxamide

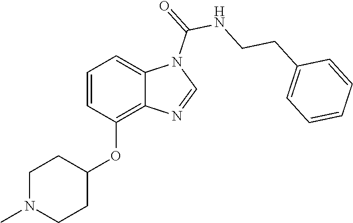 [SMILES: CN1CCC(Oc2cccc3c2ncn3C(=O)NCCc2ccccc2)CC1]

To a solution of 1,3-difluoro-2-nitrobenzene (1 g, 6.3 mmol) in DMF (20 mL) was added NaH (225 mg, 9.5 mmol) and 1-methylpiperidin-4-ol (870 mg, 7.55 mmol). The mixture was stirred at RT overnight and then the resulting mixture was quenched with water (30 mL), extracted with DCM (30 mL×3) and concentrated in vacuo to give a residue which was purified by silica gel column chromatography (DCM/MeOH=10/1) to give 4-(3-fluoro-2-nitrophenoxy)-1-methylpiperidine (1.0 g, 62.9%) as a green oil. LC-MS m/z: 255.2 [M+H]⁺. HPLC Purity (214 nm): 97%; t_{R}=0.40 min

The solution of 4-(3-fluoro-2-nitrophenoxy)-1-methylpiperidine (1 g, 4 mmol) in NH₃ in MeOH (10 mL) was stirred at 100° C. for 15 h. The resulting mixture was concentrated in vacuo to give 3-(1-methylpiperidin-4-yloxy)-2-nitroaniline (600 mg, 60.7%) as a brown oil. LC-MS m/z: 252.2 [M+H]⁺. HPLC Purity (214 nm): 91%; t_{R}=0.22 min.

Following general procedure F, 3-(1-methylpiperidin-4-yloxy)-2-nitroaniline (500 mg, 2 mmol) afforded 3-(1-methylpiperidin-4-yloxy)benzene-1,2-diamine (400 mg, 91.0%) as a brown oil. LC-MS m/z: 222.0 [M+H]⁺. HPLC Purity (254 nm): 97%; t_{R}=1.48 min.

Following general procedure G (method A), 3-(1-methylpiperidin-4-yloxy)benzene-1,2-diamine (400 mg, 1.8 mmol) afforded 4-(1-methylpiperidin-4-yloxy)-1H-benzo[d]imidazole (370 mg, 81.3%) as a yellow oil. LC-MS m/z: 232.2 [M+H]⁺. HPLC Purity (214 nm): 90%; t_{R}=0.78 min.

Following general procedure B, 4-(1-methylpiperidin-4-yloxy)-1H-benzo[d]imidazole (370 mg, 1.6 mmol) and (2-isocyanatoethyl)benzene (353 mg, 2.4 mmol) afforded the title compound (154 mg, 25.5%) as a white solid. ¹H NMR (400 MHz, MeOD) δ 8.36 (s, 1H), 7.54 (d, J=7.7 Hz, 1H), 7.25 (dd, J=13.5, 7.4 Hz, 7H), 6.87 (d, J=7.9 Hz, 1H), 4.72 (s, 1H), 3.64-3.57 (m, 2H), 2.95 (t, J=7.4 Hz, 2H), 2.80 (brs, 2H), 2.39 (brs, 3H), 2.30 (s, 3H), 2.04 (brs, 2H), 1.92 (brs, 2H). LC-MS m/z: 379.3 [M+H]⁺. HPLC Purity (214 nm): 98.08%; t_{R}=6.80 min.

### Example 87—3-Methyl-2-oxo-N-phenethyl-2,3-dihydro-1H-imidazo[4,5-b]pyridine-1-carboxamide

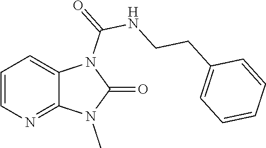 [SMILES: Cn1c(=O)n(C(=O)NCCc2ccccc2)c2cccnc21]

Following general procedure C, 3-methyl-1H-imidazo[4,5-b]pyridin-2(3H)-one (170 mg, 1.14 mmol) and 2-phenylethanamine (276.52 mg, 2.28 mmol) afforded the title compound (44.4 mg, 13.0%) as a white solid. ¹H NMR (400 MHz, CDCl₃) δ 8.62 (s, 1H), 8.32 (d, J=8.0 Hz, 1H), 8.16 (d, J=4.8 Hz, 1H), 7.35-7.22 (m, 5H), 7.12-7.09 (m, 1H), 3.71-3.66 (m, 2H), 3.50 (s, 3H), 2.96 (t, J=7.2 Hz, 2H). LC-MS m/z: 297.1 [M+H]⁺. HPLC Purity (214 nm): 98.45%; t_{R}=9.06 min.

### Example 88—1-Methyl-2-oxo-N-phenethyl-1,2-dihydro-3H-imidazo[4,5-b]pyridine-3-carboxamide

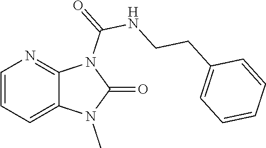 [SMILES: Cn1c(=O)n(C(=O)NCCc2ccccc2)c2ncccc21]

Following general procedure C, 3-methyl-1H-imidazo[4,5-b]pyridin-2(3H)-one (170 mg, 1.14 mmol) and 2-phenylethanamine (276.52 mg, 2.28 mmol) afforded the title compound (7.0 mg, 2.1%) as a white solid. ¹H NMR (400 MHz, CDCl₃) δ 8.80 (s, 1H), 8.17 (dd, J=5.2, 1.6 Hz, 1H), 7.34-7.22 (m, 6H), 7.17-7.13 (m, 1H), 3.73-3.68 (m, 2H), 3.42 (s, 3H), 2.97 (t, J=7.2 Hz, 2H). LC-MS m/z: 297.1 [M+H]⁺. HPLC Purity (214 nm): 100%; t_{R}=7.81 min.

### Example 89—2-(2-(Dimethylamino)ethoxy)-N-phenethyl-1H-benzo[d]imidazole-1-carboxamide

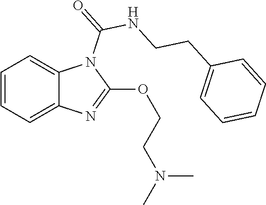 [SMILES: CN(C)CCOc1nc2ccccc2n1C(=O)NCCc1ccccc1]

A mixture of 2-chloro-1-((2-(trimethylsilyl)ethoxy)methyl)-1H-benzo[d]imidazole (500 mg, 1.77 mmol), 2-(dimethylamino)ethanol (205 mg, 2.3 mmol) and tBuONa (340 mg, 3.55 mmol) in DMF (5 mL) was stirred at 60° C. for 2 h. The reaction was treated with water (30 mL), extracted with EA (3×30 mL) and the combined organic layers were concentrated. The residue was purified by silica gel column chromatography (DCM:MeOH=20:1) to give N,N-dimethyl-2-(1-((2-(trimethylsilyl)ethoxy)methyl)-1H-benzo[d]imidazol-2-yloxy)ethanamine (500 mg, 84%) as a yellow solid. LC-MS m/z: 336.0 [M+H]⁺. HPLC Purity (254 nm): >99%; t_{R}=1.68 min.

A solution of N,N-dimethyl-2-(1-((2-(trimethylsilyl)ethoxy)methyl)-1H-benzo[d]imidazol-2-yloxy)ethanamine (500 mg, 1.49 mmol) in nBu₄NF (1M in THF, 10 mL) was stirred at 60° C. for 15 h. The reaction was concentrated and purified by silica gel column chromatography (DCM:MeOH=10:1) to give 2-(1H-benzo[d]imidazol-2-yloxy)-N,N-dimethylethanamine (230 mg, 75%) as a yellow oil. LC-MS m/z: 206.1 [M+H]⁺. HPLC Purity (254 nm): >99%; t_{R}=1.45 min.

Following general procedure B, 2-(1H-benzo[d]imidazol-2-yloxy)-N,N-dimethylethanamine (100 mg, 0.49 mmol) and (2-isocyanatoethyl)benzene (143 mg, 0.98 mmol) afforded the title compound (17 mg, 9.9%) as a white solid. ¹H NMR (400 MHz, CDCl₃) δ 8.19-8.15 (m, 1H), 7.76 (s, 1H), 7.51-7.48 (m, 1H), 7.37-7.32 (m, 2H), 7.25-7.20 (m, 5H), 4.67-4.58 (m, 2H), 3.67 (dt, J=12.8, 7.0 Hz, 2H), 2.95 (t, J=7.0 Hz, 2H), 2.59 (t, J=5.5 Hz, 2H), 2.19 (s, 6H). LC-MS m/z: 353.0 [M+H]⁺. HPLC Purity (214 nm): >99%; t_{R}=1.79 min.

### Example 90—4-iso-Propoxy-N-phenethyl-1H-benzo[d]imidazole-1-carboxamide

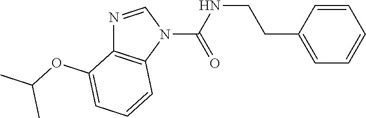 [SMILES: CC(C)Oc1cccc2c1ncn2C(=O)NCCc1ccccc1]

A mixture of 2-amino-3-nitrophenol (154 mg, 1.0 mmol), 2-iodopropane (170 mg, 1.0 mmol) and K₂CO₃ (276 mg, 2.0 mmol) in DMF (2 mL) was stirred at 60° C. for 15 h. The reaction was quenched with water (30 mL), extracted with EA (2×30 mL) and the combined organic layers were concentrated to give 2-isopropoxy-6-nitroaniline (180 mg, 92%) as a yellow oil. LC-MS m/z: 197.4 [M+H]⁺. HPLC purity (254 nm): >97%; t_{R}=1.07 min.

Following general procedure F, 2-isopropoxy-6-nitroaniline (180 mg, 0.92 mmol) afforded 3-isopropoxybenzene-1,2-diamine (140 mg, 92%) as a yellow oil. LC-MS m/z: 167.4 [M+H]⁺. HPLC purity (254 nm): >90%; t_{R}=0.63 min.

Following general procedure G (method A), 3-isopropoxybenzene-1,2-diamine (140 mg, 0.84 mmol) afforded 4-isopropoxy-1H-benzo[d]imidazole (100 mg, 68%) as a yellow solid. LC-MS m/z: 177.3 [M+H]⁺. HPLC purity (254 nm): >99%; t_{R}=0.66 min.

Following general procedure B, 4-isopropoxy-1H-benzo[d]imidazole (100 mg, 0.57 mmol) and (2-isocyanatoethyl)benzene (125 mg, 0.85 mmol) afforded the title compound (88.6 mg, 48.2%) as a white solid. ¹H NMR (400 MHz, CDCl₃) δ 8.25 (s, 1H), 7.36 (t, J=7.2 Hz, 2H), 7.31-7.26 (m, 3H), 7.20 (t, J=8.2 Hz, 1H), 7.02 (t, J=7.8 Hz, 1H), 6.78 (d, J=8.1 Hz, 1H), 5.85 (s, 1H), 4.90 (dt, J=12.2, 6.1 Hz, 1H), 3.80 (dd, J=12.6, 6.7 Hz, 2H), 3.02 (t, J=6.8 Hz, 2H), 1.43 (d, J=6.1 Hz, 6H). LC-MS m/z: 324.1 [M+H]⁺. HPLC Purity (214 nm): >96%; t_{R}=2.02 min.

### Examples 91a and 91b—2-Ethoxy-N-(3-phenylpropyl)-1H-imidazo[4,5-b]pyridine-1-carboxamide and 2-ethoxy-N-(3-phenylpropyl)-3H-imidazo[4,5-b]pyridine-3-carboxamide

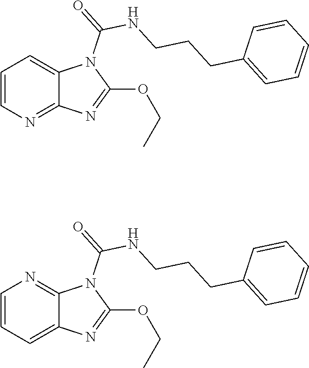 [SMILES: CCOc1nc2ncccc2n1C(=O)NCCCc1ccccc1 ; CCOc1nc2cccnc2n1C(=O)NCCCc1ccccc1]

A mixture of pyridine-2,3-diamine (1 g, 9.16 mmol) in C(OEt)₄ (5.5 mL) was stirred at 130° C. for 2 h. The mixture was cooled and diluted with EA (10 mL), filtered and concentrated to give 2-ethoxy-1H-imidazo[4,5-b]pyridine (530 mg, 35%) as a gray solid. LC-MS m/z: 164.0 [M+H]⁺. HPLC Purity (214 nm): 100%; t_{R}=1.47 min.

Following general procedure C, 2-ethoxy-1H-imidazo[4,5-b]pyridine (300 mg, 1.84 mmol) and 3-phenylpropan-1-amine (249 mg, 5.52 mmol) afforded 2-ethoxy-N-(3-phenylpropyl)-1H-imidazo[4,5-b]pyridine-1-carboxamide (83.2 mg, 13.9%/o) and 2-ethoxy-N-(3-phenylpropyl)-3H-imidazo[4,5-b]pyridine-3-carboxamide (144 mg, 24.1%) as white solids.

2-ethoxy-N-(3-phenylpropyl)-1H-imidazo[4,5-b]pyridine-1-carboxamide: ¹H NMR (400 MHz, CDCl₃) δ 9.60 (s, 1H), 8.13 (d, J=4.4 Hz, 1H), 7.77 (d, J=7.6 Hz, 1H), 7.32-7.15 (m, 6H), 4.72 (q, J=7.1 Hz, 2H), 3.54-3.48 (m, 2H), 2.77 (t, J=7.6 Hz, 2H), 2.04 (p, J=7.2 Hz, 2H), 1.58 (t, J=7.2 Hz, 3H). LC-MS m/z: 325.1 [M+H]⁺. HPLC Purity (214 nm): 100%; t_{R}=9.29 min.

2-ethoxy-N-(3-phenylpropyl)-3H-imidazo[4,5-b]pyridine-3-carboxamide: ¹H NMR (400 MHz, CDCl₃) δ 8.38 (dt, J=7.8, 1.6 Hz, 2H), 7.34-7.28 (m, 2H), 7.24-7.20 (m, 3H), 7.10 (dd, J=4.8, 4.0 Hz, 1H) 7.03 (brs, 1H), 4.84 (q, J=7.1 Hz, 2H), 3.53-3.46 (m, 2H), 2.74 (t, J=7.6 Hz, 2H), 2.00 (p, J=7.2 Hz, 2H), 1.59 (t, J=6.8 Hz, 3H). LC-MS m/z: 325.1 [M+H]⁺. HPLC Purity (214 nm): 100%; t_{R}=7.42 min.

### Example 92—2-Ethoxy-4-fluoro-N-phenethyl-1H-benzo[d]imidazole-1-carboxamide

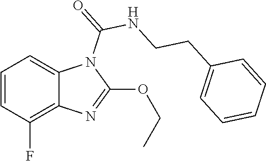 [SMILES: CCOc1nc2c(F)cccc2n1C(=O)NCCc1ccccc1]

Following general procedure G (method A), 3-fluorobenzene-1,2-diamine (500 mg, 3.0 mmol) afforded 2-ethoxy-4-fluoro-1H-benzo[d]imidazole (200 mg, 37%) as a yellow solid. LC-MS m/z: 181 [M+H]⁺. HPLC Purity (214 nm): 76%; t_{R}=1.73 min.

Following general procedure C, 2-ethoxy-4-fluoro-1H-benzo[d]imidazole (145 mg, 0.8 mmol) and 3-phenylpropan-1-amine (57 mg, 0.42 mmol) afforded the title compound (22.5 mg, 8.6%) as a white solid. ¹H NMR (400 MHz, CDCl₃) δ 7.81 (d, J=1.3 Hz, 1H), 7.21-7.11 (m, 5H), 7.01-6.98 (m, 1H), 6.88-6.79 (m, 2H), 4.52 (q, J=6.8 Hz, 2H), 3.49 (dt, J=7.5, 5.8 Hz, 2H), 2.84 (t, J=6.4 Hz, 2H), 1.19 (t, J=7.2 Hz, 3H). LC-MS m/z: 328 [M+H]⁺. HPLC Purity (214 nm): 99%; t_{R}=2.15 min.

### Example 93—4-Chloro-2-ethoxy-N-phenethyl-1H-benzo[d]imidazole-1-carboxamide

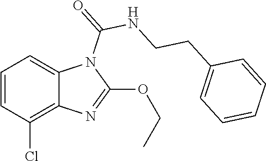 [SMILES: CCOc1nc2c(Cl)cccc2n1C(=O)NCCc1ccccc1]

A solution of 3-chlorobenzene-1,2-diamine (2.1 g, 14.7 mmol), C(OEt)₄ (4.23 g, 22.0 mmol) and TEA (2.22 g, 22.05 mmol) in HOAc (10 mL) was stirred at 80° C. overnight. The reaction mixture was poured into water (30 mL), extracted with DCM (50 mL×2), dried over NaSO₄, concentrated in vacuo and purified by silica gel column chromatography (MeOH:DCM=1:10) to afford 4-chloro-2-ethoxy-1H-benzo[d]imidazole (2.5 g, 86.5%) as a yellow oil. LC-MS m/z: 196.0 [M+H]⁺. HPLC Purity (254 nm): 97.78%; t_{R}=2.10 min.

Following general procedure B, 4-chloro-2-ethoxy-1H-benzo[d]imidazole (0.1 g, 0.51 mmol) and (2-isocyanatoethyl)benzene (0.12 g, 0.76 mmol) afforded the title compound (43.8 mg, 12.5%) as a white solid. ¹H NMR (400 MHz, CDCl₃) δ 8.10 (dd, J=8.1, 0.9 Hz, 1H), 7.35 (t, J=7.2 Hz, 2H), 7.31-7.27 (m, 2H), 7.25 (s, 2H), 7.14 (t, J=8.1 Hz, 1H), 7.07-7.02 (m, 1H), 4.67 (q, J=7.1 Hz, 2H), 3.77 (dt, J=6.4, 6.0 Hz, 2H), 2.96 (t, J=6.8 Hz, 2H), 1.30 (t, J=7.1 Hz, 3H). LC-MS m/z: 343.1 [M+H]⁺. HPLC Purity (254 nm): 100%; t_{R}=10.44 min.

### Example 94—N-Butyl-2-ethoxy-4-methoxy-1H-benzo[d]imidazole-1-carboxamide

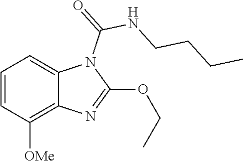 [SMILES: CCCCNC(=O)n1c(OCC)nc2c(OC)cccc21]

To a solution of 3-methoxybenzene-1,2-diamine (500 mg, 3.62 mmol) in AcOH (5 mL) was added C(OEt)₄ (3 mL) and the mixture was stirred at RT for 4 h. The mixture was purified by silica gel column chromatography (PE:EA=2:1) to give 2-ethoxy-4-methoxy-1H-benzo[d]imidazole (570 mg, 81.9%) as a light brown solid. LC-MS m/z: 193.3 [M+H]⁺. HPLC Purity (214 nm): 100%, t_{R}=0.70 min.

Following general procedure B, 2-ethoxy-4-methoxy-1H-benzo[d]imidazole (200 mg, 1.04 mmol) and 1-isocyanatobutane (206 mg, 2.08 mmol) afforded the title compound (213.1 mg, 70.3%) as a white solid. ¹H NMR (400 MHz, CDCl₃) δ 7.80 (d, J=8.2 Hz, 1H), 7.15 (t, J=8.2 Hz, 1H), 7.08 (brs, 1H), 6.76 (d, J=8.2 Hz, 1H), 4.80 (q, J=7.1 Hz, 2H), 3.99 (s, 3H), 3.45 (dt, J=12.5, 6.9 Hz, 2H), 1.68-1.58 (m, 2H), 1.54 (t, J=7.1 Hz, 3H), 1.47 (dq, J=14.4, 7.2, 2H), 0.98 (t, J=7.2 Hz, 3H). LC-MS m/z: 292.1 [M+H]⁺. HPLC Purity (214 nm): 98.72%; t_{R}=9.13 min.

### Example 95—2-Methoxy-N-(2-methoxyethyl)-1H-imidazo[4,5-b]pyridine-1-carboxamide

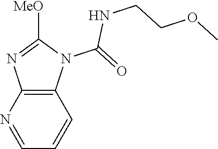 [SMILES: COCCNC(=O)n1c(OC)nc2ncccc21]

Following general procedure C, 2-methoxy-1H-imidazo[4,5-b]pyridine (100 mg, 0.67 mmol) and 2-methoxyethanamine (79.8 mg, 1.01 mmol) afforded the title compound (2.7 mg, 1.6%) as a yellow solid. ¹H NMR (400 MHz, CDCl₃) δ 9.77 (s, 1H), 8.17 (d, J=4.9 Hz, 1H), 7.81 (d, J=7.9 Hz, 1H), 7.25-7.18 (m, 1H), 4.29 (s, 3H), 3.73-3.66 (m, 2H), 3.65-3.60 (m, 2H), 3.43 (s, 3H). LC-MS m/z: 250.11 [M+H]⁺. HPLC Purity (214 nm): 95.20%; t_{R}=6.45 min.

### Example 96—N-Butyl-2-isopropoxy-1H-benzo[d]imidazole-1-carboxamide

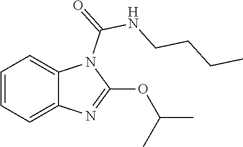 [SMILES: CCCCNC(=O)n1c(OC(C)C)nc2ccccc21]

To a solution of 2-chloro-1H-benzo[d]imidazole (3 g, 19.7 mmol) in DMF (90 mL) was added NaH (708 mg, 29.5 mmol) and the mixture was stirred at 0° C. for 30 min. Then SEMCl (4.9 g, 29.5 mmol) was added and the reaction mixture was stirred at 60° C. overnight. The mixture was quenched with water (100 mL) and extracted with EA (50 mL×3). The combined organic layers was concentrated in vacuo to give a residue which was purified by silica gel column chromatography (PE:EA=8:1) affording 2-chloro-1-((2-(trimethylsilyl)ethoxy)methyl)-1H-benzo[d]imidazole (5 g, 90.1%) as a white solid. LC-MS m/z: 283.3 [M+H]⁺. HPLC Purity (214 nm): 88.47%; t_{R}=1.37 min.

To a flask containing propan-2-ol was added NaH (84 mg, 3.5 mmol) and the resulting reaction mixture was stirred at 0° C. for 30 min. Then 2-chloro-1-((2-(trimethylsilyl)ethoxy)methyl)-1H-benzo[d]imidazole (500 mg, 1.8 mmol) was added and stirred at 60° C. overnight. The resulting mixture was concentrated in vacuo to give a residue which was purified by silica gel column chromatography (PE:EA=9:1) to give 2-isopropoxy-1-((2-(trimethylsilyl)ethoxy)methyl)-1H-benzo[d]imidazole (450 mg, 83%) as a yellow oil. LC-MS m/z: 307.3 [M+H]⁺. HPLC Purity (254 nm): 100%; t_{R}=1.38 min.

A solution of 2-isopropoxy-1-((2-(trimethylsilyl)ethoxy)methyl)-1H-benzo[d]imidazole (450 mg, 1.5 mmol) in nBu₄NF (1M in THF, 10 mL) was stirred at 60° C. for 15 hrs. The resulting mixture was concentrated and purified by silica gel column chromatography (PE/EA=1:1) to give 2-isopropoxy-1H-benzo[d]imidazole (300 mg, 69.2%) as a white solid. LC-MS m/z: 177.3 [M+H]⁺. HPLC Purity (214 nm): 60.79%; t_{R}=0.65 min.

Following general procedure B, 2-isopropoxy-1H-benzo[d]imidazole (300 mg, 1.7 mmol) and 1-isocyanatobutane (253 mg, 2.5 mmol) afforded the title compound (54.4 mg, 11.8%) as a white solid. ¹H NMR (400 MHz, CDCl₃) δ 8.19-8.13 (m, 1H), 7.47 (dd, J=6.7, 2.2 Hz, 1H), 7.27-7.18 (m, 2H), 7.10 (s, 1H), 5.54 (septet, J=6.2 Hz, 1H), 3.48 (dt, J=6.8, 1.2 Hz, 2H), 1.65-1.60 (m, 2H), 1.54 (d, J=6.2 Hz, 6H), 1.45 (dq, J=15.1, 7.4 Hz, 2H), 0.99 (t, J=7.2 Hz, 3H). LC-MS m/z: 276.0 [M+H]⁺. HPLC Purity (214 nm): 100%; t_{R}=10.13 min.

### Example 97—N-Butyl-2-ethoxy-1H-benzo[d]imidazole-1-carboxamide

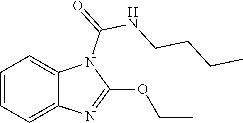 [SMILES: CCCCNC(=O)n1c(OCC)nc2ccccc21]

Following general procedure B, 2-ethoxy-1H-benzo[d]imidazole (0.10 g, 0.62 mmol) and 1-isocyanatobutane (0.07 g, 0.74 mmol) afforded the title compound (84.3 mg, 52.6%) as a white solid. ¹H NMR (400 MHz, DMSO-di) δ 7.86 (brs, 1H), 7.80 (d, J=7.2 Hz, 1H), 7.43 (d, J=7.2 Hz, 1H), 7.19-7.14 (m, 2H), 4.60 (dd, J=14 Hz, 6.7 Hz, 2H), 3.33-3.27 (m, 2H), 1.60-1.52 (m, 2H), 1.44 (t, J=12.5 Hz, 3H), 1.41-1.34 (m, 2H), 0.92 (t, J=6.8 Hz, 3H). LC-MS m/z: 262.2 [M+H]⁺. HPLC Purity (214 nm): 100%; t_{R}=7.31 min.

### Example 98—N-iso-Pentyl-4-methoxy-1H-benzo[d]imidazole-1-carboxamide

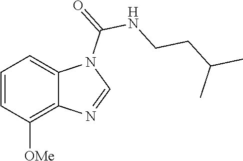 [SMILES: COc1cccc2c1ncn2C(=O)NCCC(C)C]

A solution of 3-methoxybenzene-1,2-diamine (1 g, 7.24 mmol) in AcOH (20 mL) was stirred at 85° C. overnight. The reaction mixture was concentrated under reduced pressure and the residue was purified by silica gel column chromatography (20% MeOH in DCM) to afford 4-methoxy-1H-benzo[d]imidazole (230 mg, 21.4%) as a white oil. LC-MS m/z: 148.06. [M+H]⁺. HPLC Purity (214 nm)=81.86%; t_{R}=1.40 min.

Following general procedure C, 4-methoxy-1H-benzo[d]imidazole (150 mg, 1.01 mmol) and 3-methylbutan-1-amine (105 mg, 1.25 mmol) afforded the title compound (72.4 mg, 27.4%) as a white solid. ¹H NMR (400 MHz, CDCl₃) δ 8.33 (s, 1H), 7.39-7.30 (m, 2H), 6.80 (d, J=7.8 Hz, 1H), 5.87 (s, 1H), 4.03 (s, 3H), 3.54 (dd, J=14.6, 6.0 Hz, 2H), 1.73 (septet, J=6.8 Hz, 1H), 1.63-1.55 (m, 2H), 0.99 (d, J=6.4 Hz, 6H). LC-MS m/z: 261.15. [M+H]⁺. HPLC Purity (254 nm)=100%; t_{R}=8.30 min.

### Example 100—N-Butyl-2,4-dimethoxy-1H-benzo[d]imidazole-1-carboxamide

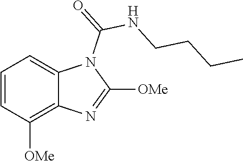 [SMILES: CCCCNC(=O)n1c(OC)nc2c(OC)cccc21]

Following general procedure G (method A), 3-methoxybenzene-1,2-diamine (500 mg, 3.62 mmol) afforded 2,4-dimethoxy-1H-benzo[d]imidazole (504 mg, 100%) as a white solid. LC-MS m/z: 179.7 [M+H]⁺. HPLC Purity (214 nm): 89%; t_{R}=1.61 min.

Following general procedure B, 2,4-dimethoxy-1H-benzo[d]imidazole (200 mg, 1.1 mmol) and 1-isocyanatobutane (149 mg, 1.7 mmol) afforded the title compound (150.0 mg, 48.2%) as a white solid. ¹H NMR (400 MHz, CDCl₃) δ 7.80 (d, J=8.2 Hz, 1H), 7.17 (t, J=8.2 Hz, 1H), 6.98 (s, 1H), 6.77 (d, J=8.2 Hz, 1H), 4.36 (s, 3H), 4.00 (s, 3H), 3.44 (dd, J=13.0, 6.9 Hz, 2H), 1.64 (dt, J=14.9, 7.4 Hz, 2H), 1.43 (dt, J=14.5, 7.3 Hz, 2H), 0.98 (t, J=7.3 Hz, 3H). LC-MS m/z: 278.7 [M+H]⁺. HPLC Purity (214 nm): 97.27%; t_{R}=7.88 min.

### Examples 101a and 101b—N-Butyl-6-(4-morpholinopiperidin-1-yl)-1H-benzo[d]imidazole-1-carboxamide and N-butyl-5-(4-morpholinopiperidin-1-yl)-1H-benzo[d]imidazole-1-carboxamide

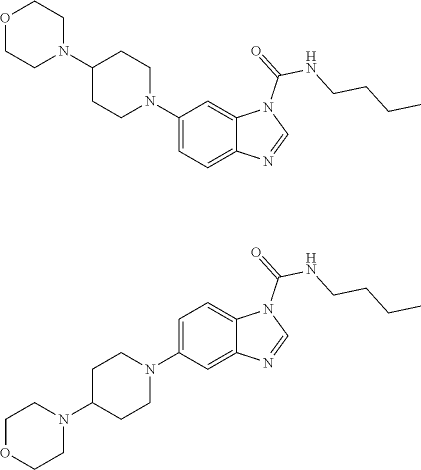 [SMILES: CCCCNC(=O)n1cnc2ccc(N3CCC(N4CCOCC4)CC3)cc21 ; CCCCNC(=O)n1cnc2cc(N3CCC(N4CCOCC4)CC3)ccc21]

A mixture of 4-fluoro-1,2-dinitrobenzene (558 mg, 3.0 mmol), 4-(piperidin-4-yl)morpholine (612 mg, 3.6 mmol) and K₂CO₃ (828 mg, 6.0 mmol) in CH₃CN (20 mL) was stirred at 60° C. for 2 h. The reaction mixture was filtered, concentrated and purified by silica gel column chromatography (DCM:MeOH=10:1) to give 4-(1-(3,4-dinitrophenyl)piperidin-4-yl) morpholine (900 mg, 900%) as a yellow solid. LC-MS m/z: 337.3 [M+H]⁺. HPLC Purity (254 nm): >92%; t_{R}=0.75 min.

Following general procedure F, 4-(1-(3,4-dinitrophenyl)piperidin-4-yl)morpholine (900 mg, 2.68 mmol) afforded 4-(4-morpholinopiperidin-1-yl)benzene-1,2-diamine (700 mg, 95%) as a yellow oil. LC-MS m/z: 277.1 [M+H]⁺. HPLC Purity (214 nm): >65%; t_{R}=1.23 min.

Following general procedure G (method A), 4-(4-morpholinopiperidin-1-yl)benzene-1,2-diamine (700 mg, 2.5 mmol) afforded 4-(1-(1H-benzo[d]imidazol-6-yl)piperidin-4-yl)morpholine (600 mg, 83%) as a yellow solid. LC-MS m/z: 287.2 [M+H]⁺. HPLC Purity (214 nm): >81%; t_{R}=1.26 min.

Following general procedure B, 4-(1-(1H-benzo[d]imidazol-6-yl)piperidin-4-yl)morpholine (300 mg, 1.05 mmol) and 1-isocyanatobutane (156 mg, 1.57 mmol) afforded N-butyl-6-(4-morpholinopiperidin-1-yl)-1H-benzo[d]imidazole-1-carboxamide (19.7 mg, 4.9%) and N-butyl-5-(4-morpholinopiperidin-1-yl)-1H-benzo[d]imidazole-1-carboxamide (31.0 mg, 7.7%) as yellow solids.

N-butyl-6-(4-morpholinopiperidin-1-yl)-1H-benzo[d]imidazole-1-carboxamide: ¹H NMR (400 MHz, CDCl₃) δ 8.20 (s, 1H), 7.63 (d, J=8.9 Hz, 1H), 7.46 (d, J=1.7 Hz, 1H), 7.05 (dd, J=8.9, 1.9 Hz, 1H), 6.00 (brs, 1H), 3.77-3.72 (m, 6H), 3.50 (dd, J=13.0, 6.9 Hz, 2H), 2.79 (t, J=11.6 Hz, 2H), 2.63-2.58 (m, 4H), 2.35 (t, J=11.2 Hz, 1H), 1.97 (d, J=12.2 Hz, 2H), 1-76-1.65 (m, 4H), 1.46 (dq, J=14.5, 7.4 Hz, 2H), 0.99 (t, J=7.3 Hz, 3H). LC-MS m/z: 386.1 [M+H]⁺. HPLC Purity (214 nm): >96%; t_{R}=1.44 min.

N-butyl-5-(4-morpholinopiperidin-1-yl)-1H-benzo[d]imidazole-1-carboxamide: ¹H NMR (400 MHz, CDCl₃) δ 8.35 (s, 1H), 7.63 (d, J=9.0 Hz, 1H), 7.32 (d, J=2.2 Hz, 1H), 7.11 (dd, J=9.0, 2.3 Hz, 1H), 5.72 (s, 1H), 3.77-3.48 (m, 6H), 3.56-3.49 (m, 2H), 2.76 (dt, J=12.2, 10.0 Hz, 2H), 2.65-2.61 (m, 4H), 2.41-2.35 (m, 1H), 1.99 (d, J=12.6 Hz, 2H), 1.80-1.64 (m, 4H), 1.46 (dq, J=14.6, 7.3 Hz, 2H), 0.99 (t, J=7.3 Hz, 3H). LC-MS m/z: 386.3 [M+H]⁺. HPLC Purity (214 nm): >99%; t_{R}=1.58 min.

### Examples 102a and 102b—N-Butyl-6-(4-(4-methylpiperazin-1-yl)piperidin-1-yl)-1H-benzo[d]imidazole-1-carboxamide and N-butyl-5-(4-(4-methylpiperazin-1-yl)piperidin-1-yl)-1H-benzo[d]imidazole-1-carboxamide

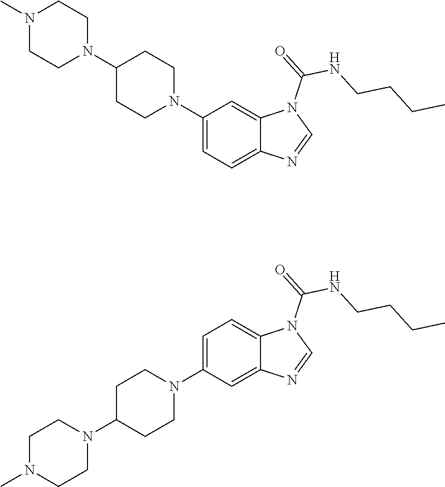

A mixture of 4-fluoro-1,2-dinitrobenzene (500 mg, 2.69 mmol), 1-methyl-4-(piperidin-4-yl)piperazine (939 mg, 3.22 mmol) and K₂CO₃ (1.8 g, 13.4 mmol) in CH₃CN (30 mL) was stirred at 60° C. for 2 h. The reaction mixture was filtered, concentrated and purified by silica gel column chromatography (DCM:MeOH=10:1) to give 1-(1-(3,4-dinitrophenyl)piperidin-4-yl)-4-methylpiperazine (600 mg, 64%) as a yellow solid. LC-MS m/z: 350.4 [M+H]⁺. HPLC Purity (254 nm): >63%; t_{R}=0.76 min.

Following general procedure F, 1-(1-(3,4-dinitrophenyl)piperidin-4-yl)-4-methylpiperazine (600 mg, 1.72 mmol) afforded 4-(4-(4-methylpiperazin-1-yl)piperidin-1-yl) benzene-1,2-diamine (490 mg, 99%) as a yellow solid. LC-MS m/z: 290.2 [M+H]⁺. HPLC Purity (254 nm): >70%; t_{R}=1.38 min.

Following general procedure G (method A), 4-(4-(4-methylpiperazin-1-yl)piperidin-1-yl)benzene-1,2-diamine (490 mg, 1.7 mmol) afforded 6-(4-(4-methylpiperazin-1-yl)piperidin-1-yl)-1H-benzo[d]imidazole (400 mg, 79%) as a yellow solid. LC-MS m/z: 300.2 [M+H]⁺. HPLC Purity (254 nm): >75%; t_{R}=1.43 min.

Following general procedure B, 6-(4-(4-methylpiperazin-1-yl)piperidin-1-yl)-1H-benzo[d]imidazole (400 mg, 1.34 mmol) and 1-isocyanatobutane (199 mg, 2.0 mmol) afforded N-butyl-6-(4-(4-methylpiperazin-1-yl)piperidin-1-yl)-1H-benzo[d]imidazole-1-carboxamide (24.9 mg, 6.2%) and N-butyl-5-(4-(4-methylpiperazin-1-yl)piperidin-1-yl)-1H-benzo[d]imidazole-1-carboxamide (31.6 mg, 7.9%) as yellow solids.

N-butyl-6-(4-(4-methylpiperazin-1-yl)piperidin-1-yl)-1H-benzo[d]imidazole-1-carboxamide: ¹H NMR (400 MHz, CDCl₃) δ 8.17 (s, 1H), 7.64 (d, J=8.9 Hz, 1H), 7.41 (d, J=2.0 Hz, 1H), 7.04 (dd, J=8.8, 2.0 Hz, 1H), 5.68 (s, 1H), 3.76 (d, J=12.2 Hz, 2H), 3.51 (dd, J=13.0, 7.0 Hz, 2H), 2.79 (t, J=11.4 Hz, 2H), 2.69-2.60 (m, 4H), 2.58-2.38 (m, 5H), 2.30 (s, 3H), 2.04-1.91 (m, 2H), 1.74-1.64 (m, 4H), 1.47 (dd, J=15.0, 7.4 Hz, 2H), 1.00 (t, J=7.3 Hz, 3H). LC-MS m/z: 399.2 [M+H]⁺. HPLC Purity (214 nm): >99%; t_{R}=1.79 min.

N-butyl-5-(4-(4-methylpiperazin-1-yl)piperidin-1-yl)-1H-benzo[d]imidazole-1-carboxamide: ¹H NMR (400 MHz, CDCl₃) δ 8.34 (s, 1H), 7.61 (d, J=8.9 Hz, 1H), 7.31 (s, 1H), 7.10 (d, J=8.9 Hz, 1H), 5.69 (s, 1H), 3.72 (d, J=12.4 Hz, 2H), 3.51 (dd, J=13.0, 6.8 Hz, 2H), 2.75 (t, J=11.4 Hz, 2H), 2.65-2.58 (m, 4H), 2.49-2.39 (m, 5H), 2.30 (s, 3H), 1.97 (d, J=11.4 Hz, 2H), 1.81-1.55 (m, 4H), 1.46 (dq, J=14.5, 7.4 Hz, 2H), 0.99 (t, J=7.3 Hz, 3H). LC-MS m/z: 399.2 [M+H]⁺. HPLC Purity (214 nm): >96%; t_{R}=1.81 min.

### Examples 103a and 103b—N-iso-Pentyl-6-(pyrimidin-5-yl)-1H-benzo[d]imidazole-1-carboxamide and N-iso-pentyl-5-(pyrimidin-5-yl)-1H-benzo[d]imidazole-1-carboxamide

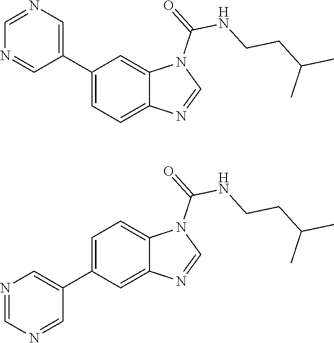 [SMILES: CC(C)CCNC(=O)n1cnc2ccc(-c3cncnc3)cc21 ; CC(C)CCNC(=O)n1cnc2cc(-c3cncnc3)ccc21]

Following general procedure C, 5-(pyrimidin-5-yl)-1H-benzo[d]imidazole (180 mg, 0.92 mmol) and 3-methylbutan-1-amine (80 mg, 0.92 mmol) afforded N-iso-pentyl-6-(pyrimidin-5-yl)-1H-benzo[d]imidazole-1-carboxamide (5.3 mg, 1.9%) and N-iso-pentyl-5-(pyrimidin-5-yl)-1H-benzo[d]imidazole-1-carboxamide (12.5 mg, 4.4%) as white solids.

N-iso-pentyl-6-(pyrimidin-5-yl)-1H-benzo[d]imidazole-1-carboxamide: ¹H NMR (400 MHz, CDCl₃) δ 9.16 (s, 1H), 8.94 (s, 2H), 8.32 (s, 1H), 8.14 (s, 1H), 7.88 (d, J=8.3 Hz, 1H), 7.50 (d, J=8.4 Hz, 1H), 5.75 (s, 1H), 3.52-3.40 (m, 2H), 1.65 (septet, J=6.8 Hz, 1H), 1.57-1.52 (m, 2H), 0.93 (d, J=6.6 Hz, 6H). LC-MS m/z: 310.7 [M+H]⁺. HPLC Purity (214 nm): 97.78%; t_{R}=6.83 min.

N-iso-pentyl-5-(pyrimidin-5-yl)-1H-benzo[d]imidazole-1-carboxamide: ¹H NMR (400 MHz, CDCl₃) δ 9.23 (s, 1H), 9.02 (s, 2H), 8.44 (s, 1H), 8.05-8.01 (m, 2H), 7.63 (dd, J=8.6, 1.5 Hz, 1H), 5.76 (s, 1H), 3.59-3.55 (m, 2H), 1.72 (septet, J=7.2 Hz, 1H), 1.67-1.59 (m, 2H), 1.01 (d, J=6.4 Hz, 6H). LC-MS m/z: 310.7 [M+H]⁺. HPLC Purity (214 nm): 98.76%; t_{R}=6.95 min.

### Example 104—N-iso-Pentyl-1-methyl-2-oxo-1,2-dihydro-3H-imidazo[4,5-b]pyridine-3-carboxamide

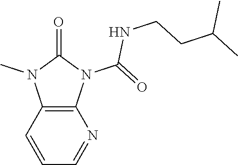 [SMILES: CC(C)CCNC(=O)n1c(=O)n(C)c2cccnc21]

Following general procedure F, N-methyl-2-nitropyridin-3-amine (1.0 g, 6.53 mmol) afforded N-methylpyridine-2,3-diamine (800 mg, 99%) as a brown oil. LC-MS m/z: 124.2, [M+H]⁺. Purity (214 nm): 97%; t_{R}=1.14 min.

To a solution of N-methylpyridine-2,3-diamine (1 g, 8.13 mmol), CDI (3.95 g, 24.3 mmol) in DCM (10 mL) was added Et₃N (1.64 g, 16.3 mmol) and the reaction mixture was stirred at RT for 1 h. Then the reaction mixture was concentrated and the residue was purified by silica gel column chromatography (13% MeOH in DCM) to give 1-methyl-1,3-dihydro-2H-imidazo[4,5-b]pyridin-2-one as an orange solid (780 mg, 65%). LC-MS m/z: 150.1. [M+H]⁺. Purity (214 nm): 68.2%; t_{R}=1.37 min.

Following general procedure C, 1-methyl-1,3-dihydro-2H-imidazo[4,5-b]pyridin-2-one (200 mg, 1.34 mmol) and 3-methylbutan-1-amine (175 mg, 2.01 mmol) afforded the title compound (158 mg, 45.2%) as a white solid. ¹H NMR (400 MHz, CDCl₃) δ 8.67 (s, 1H), 8.23 (dd, J=4.8 Hz, 3.6 Hz, 1H), 7.24 (d, J=1.2 Hz, 1H), 7.17-7.14 (m, 1H), 3.49-3.42 (m, 5H), 1.74-1.68 (m, 1H), 1.57-1.52 (m, 2H), 0.95 (d, J=6.4 Hz, 6H). LC-MS m/z: 263.0 [M+H]⁺. HPLC Purity (214 nm): 93%; t_{R}=7.97 min.

### Example 105—N-iso-Pentyl-3-methyl-2-oxo-2,3-dihydro-1H-imidazo[4,5-b]pyridine-1-carboxamide

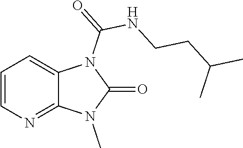 [SMILES: CC(C)CCNC(=O)n1c(=O)n(C)c2ncccc21]

Following general procedure C, 3-methyl-1H-imidazo[4,5-b]pyridin-2(3H)-one (150 mg, 1.0 mmol) and 3-methylbutan-1-amine (175.5 mg, 2.0 mmol) afforded the title compound (2.4 mg, 0.9%) as a white solid. ¹H NMR (400 MHz, CDCl₃) δ 8.54 (s, 1H), 8.31 (d, J=8.0 Hz, 1H), 8.16 (d, J=5.2 Hz, 1H), 7.12-7.09 (m, 1H), 3.52 (s, 3H), 3.49-3.42 (m, 2H), 1.73-1.69 (m, 1H), 1.51-1.47 (m, 2H), 0.96 (d, J=6.4 Hz, 6H). LC-MS m/z: 263.0 [M+H]⁺. HPLC Purity (214 nm): 98.31%; t_{R}=9.42 min.

### Example 106—N-iso-Pentyl-2-methoxy-6-(pyrimidin-5-yl)-1H-benzo[d]imidazole-1-carboxamide

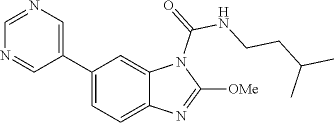 [SMILES: COc1nc2ccc(-c3cncnc3)cc2n1C(=O)NCCC(C)C]

Following general procedure C, 2-methoxy-6-(pyrimidin-5-yl)-1H-benzo[d]imidazole (150 mg, 0.66 mmol) and 3-methylbutan-1-amine (69 mg, 0.79 mmol) afforded the title compound (35.9 mg, 16.1%) as a white solid. ¹H NMR (400 MHz, CDCl₃) δ 9.19 (s, 1H), 8.99 (s, 2H), 8.31 (d, J=8.4 Hz, 1H), 7.72 (s, 1H), 7.45 (d, J=8.4 Hz, 1H), 6.94 (t, J=5.2 Hz, 1H), 4.37 (s, 3H), 3.48 (q, J=7.2 Hz, 2H), 1.75-1.64 (m, 3H), 1.00 (d, J=6.8 Hz, 6H). LC-MS m/z: 340.2 [M+H]⁺. HPLC Purity (214 nm): 99%; t_{R}=8.56 min.

### Example 107—N-iso-Pentyl-1H-benzo[d]imidazole-1-carboxamide

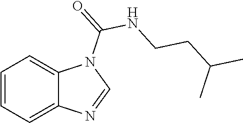 [SMILES: CC(C)CCNC(=O)n1cnc2ccccc21]

Following general procedure C, 1H-benzo[d]imidazole (300 mg, 2.53 mmol) and 3-methylbutan-1-amine (330 mg, 3.79 mmol) afforded the title compound (19.6 mg, 3.3%) as a white solid. ¹H NMR (400 MHz, CDCl₃) δ 8.41 (s, 1H), 7.82 (ddd, J=7.4, 4.4, 1.5 Hz, 2H), 7.46-7.32 (m, 2H), 5.86 (s, 1H), 3.57-3.51 (m, 2H), 1.72 (septet, J=6.8 Hz, 1H), 1.60 (dd, J=14.8, 7.2 Hz, 2H), 0.99 (d, J=6.8 Hz, 6H). LC-MS m/z: 232.1 [M+H]⁺. HPLC Purity (214 nm): 100%; t_{R}=7.81 min.

### Example 108—N-(4-Methylphenethyl)-1H-benzo[d]imidazole-1-carboxamide

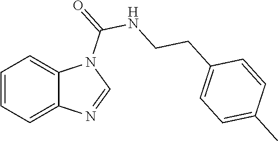 [SMILES: Cc1ccc(CCNC(=O)n2cnc3ccccc32)cc1]

Following general procedure C, 1H-benzo[d]imidazole (0.30 g, 2.54 mmol) and 2-p-tolylethanamine (0.56 g, 2.80 mmol) afforded the title compound (66.2 mg, 9.3%) as a white solid. ¹H NMR (400 MHz, CDCl₃) δ 8.32 (s, 1H), 7.80 (d, J=8.4 Hz, 1H), 7.48 (d, J=8.4 Hz, 1H), 7.36-7.26 (m, 2H), 7.18-7.13 (m, 4H), 5.71 (brs, 1H), 3.78 (dd, J=12.4, 6.4 Hz, 2H), 2.98 (t, J=6.8 Hz, 2H), 2.35 (s, 3H). LC-MS m/z: 280.2 [M+H]⁺. HPLC Purity (254 nm): 100%; t_{R}=8.40 min.

### Examples 109a and 109b—N-(sec-Butyl)-2-methoxy-6-(pyrimidin-5-yl)-1H-benzo[d]imidazole-1-carboxamide and N-(sec-butyl)-2-methoxy-5-(pyrimidin-5-yl)-1H-benzo[d]imidazole-1-carboxamide

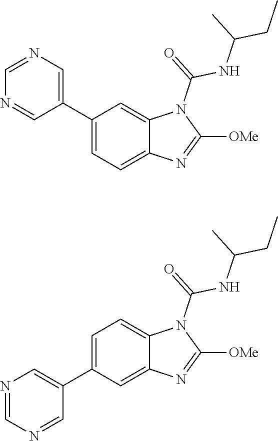 [SMILES: CCC(C)NC(=O)n1c(OC)nc2ccc(-c3cncnc3)cc21 ; CCC(C)NC(=O)n1c(OC)nc2cc(-c3cncnc3)ccc21]

Following general procedure C, 2-methoxy-6-(pyrimidin-5-yl)-1H-benzo[d]imidazole (226 mg, 1.0 mmol) and butan-2-amine (73 mg, 1.0 mmol) afforded N-(sec-butyl)-2-methoxy-6-(pyrimidin-5-yl)-1H-benzo[d]imidazole-1-carboxamide (23.1 mg, 7.1%) and N-(sec-butyl)-2-methoxy-5-(pyrimidin-5-yl)-1H-benzo[d]imidazole-1-carboxamide (16.2 mg, 4.9%) both as pale yellow solids.

N-(sec-butyl)-2-methoxy-6-(pyrimidin-5-yl)-1H-benzo[d]imidazole-1-carboxamide: ¹H NMR (400 MHz, CDCl₃) δ 9.19 (s, 1H), 8.99 (s, 2H), 8.30 (d, J=8.4 Hz, 11H), 7.72 (s, 1H), 7.45 (dd, J=6.8, 1.6 Hz, 1H), 6.76 (d, J=7.2 Hz, 1H), 4.37 (s, 3H), 4.04-4.00 (m, 1H), 1.71-1.60 (m, 2H), 1.30 (d, J=6.4 Hz, 3H), 1.01 (t, J=7.2 Hz, 3H). LC-MS m/z: 325.7 [M+H]⁺. HPLC Purity (214 nm): 96.20%; t_{R}=8.27 min.

N-(sec-butyl)-2-methoxy-5-(pyrimidin-5-yl)-1H-benzo[d]imidazole-1-carboxamide: ¹H NMR (400 MHz, CDCl₃) δ 9.17 (s, 1H), 9.00 (s, 2H), 8.46 (d, J=1.6 Hz, 1H), 7.63 (d, J=8.4 Hz, 1H), 7.47 (dd, J=6.4, 1.6 Hz, 1H), 6.77 (d, J=7.2 Hz, 1H), 4.37 (s, 3H), 4.04-4.00 (m, 1H), 1.66-1.61 (m, 2H), 1.30-1.25 (m, 3H), 1.01 (t, J=7.6 Hz, 3H). LC-MS m/z: 325.7 [M+H]⁺. HPLC Purity (214 nm): 96.20%; t_{R}=8.11 min.

### Example 110—N-iso-Pentyl-3-methyl-2-oxo-6-(pyridin-3-yl)-2,3-dihydro-1H-imidazo[4,5-b]pyridine-1-carboxamide

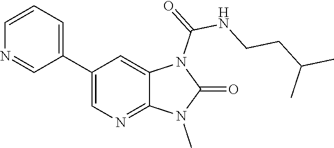 [SMILES: CC(C)CCNC(=O)n1c(=O)n(C)c2ncc(-c3cccnc3)cc21]

Following general procedure G (method B), 5-bromopyridine-2,3-diamine (7.22 g, 38.4 mmol) afforded 6-bromo-2-methoxy-1H-imidazo[4,5-b]pyridine (2.5 g, 28.6%) as a yellow solid. LC-MS m/z: 228.1 [M+H]⁺. Purity (254 nm): 61%; t_{R}=1.20 min.

Following general procedure A, 6-bromo-2-methoxy-1H-imidazo[4,5-b]pyridine (2.5 g, 11.0 mmol) and pyridin-3-ylboronic acid (1.6 g, 13.2 mmol) afforded 2-methoxy-6-(pyridin-3-yl)-1H-imidazo[4,5-b]pyridine (0.8 g, 32%) as an off white solid. LC-MS m/z: 227.1 [M+H]⁺. Purity (254 nm): 77%; t_{R}=1.20 min.

Following general procedure C, 2-methoxy-6-(pyridin-3-yl)-1H-imidazo[4,5-b]pyridine (0.2 g, 0.9 mmol) and 3-methylbutan-1-amine (0.1 g, 1.4 mmol) afforded the title compound as a white solid (16.1 mg, 1.8%). ¹H NMR (400 MHz, CDCl₃) δ 8.86 (d, J=1.9 Hz, 1H), 8.64 (d, J=3.5 Hz, 1H), 8.60 (d, J=2.0 Hz, 1H), 8.53 (s, 1H), 8.38 (d, J=2.0 Hz, 1H), 7.90 (d, J=8.0 Hz, 1H), 7.40 (dd, J=7.9, 4.8 Hz, 1H), 3.56 (s, 2H), 3.47 (dd, J=13.2, 7.2 Hz, 2H), 1.71 (dt, J=20.1, 6.7 Hz, 1H), 1.57-1.51 (m, 2H), 0.97 (d, J=6.6 Hz, 6H). LC-MS m/z: 340.2 [M+H]⁺. HPLC Purity (214 nm): 98.3%; t_{R}=6.85 min.

### Examples 11a and 11b—N-Cyclohexyl-2-methoxy-6-(pyrimidin-5-yl)-1H-benzo[d]imidazole-1-carboxamide and N-cyclohexyl-2-methoxy-5-(pyrimidin-5-yl)-1H-benzo[d]imidazole-1-carboxamide

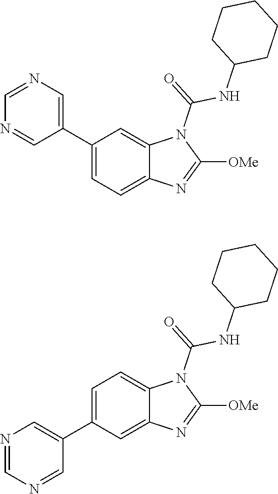 [SMILES: COc1nc2ccc(-c3cncnc3)cc2n1C(=O)NC1CCCCC1 ; COc1nc2cc(-c3cncnc3)ccc2n1C(=O)NC1CCCCC1]

Following general procedure C, 2-methoxy-6-(pyrimidin-5-yl)-1H-benzo[d]imidazole (226 mg, 1.0 mmol) and cyclohexylamine (100 mg, 1.0 mmol) afforded N-cyclohexyl-2-methoxy-6-(pyrimidin-5-yl)-1H-benzo[d]imidazole-1-carboxamide (35 mg, 9.9%) and N-cyclohexyl-2-methoxy-5-(pyrimidin-5-yl)-1H-benzo[d]imidazole-1-carboxamide (45 mg, 13.8%) both as white solids.

N-cyclohexyl-2-methoxy-6-(pyrimidin-5-yl)-1H-benzo[d]imidazole-1-carboxamide: ¹H NMR (400 MHz, CDCl₃) δ 9.19 (s, 1H), 8.99 (s, 2H), 8.30 (d, J=8.4 Hz, 1H), 7.72 (d, J=1.6 Hz, 1H), 7.45 (dd, J=6.8, 1.6 Hz, 1H), 6.87 (d, J=7.2 Hz, 1H), 4.37 (s, 3H), 3.90-3.88 (m, 1H), 2.08-2.05 (m, 2H), 1.80-1.74 (m, 2H), 1.46-1.23 (m, 6H). LC-MS m/z: 352.1 [M+H]⁺. HPLC Purity (214 nm): 91.76%; t_{R}=9.03 min.

N-cyclohexyl-2-methoxy-5-(pyrimidin-5-yl)-1H-benzo[d]imidazole-1-carboxamide: ¹H NMR (400 MHz, CDCl₃) δ 9.17 (s, 1H), 9.00 (s, 2H), 8.47 (d, J=1.2 Hz, 1H), 7.62 (d, J=8.4 Hz, 1H), 7.47 (dd, J=6.4, 1.6 Hz, 1H), 6.88 (d, J=7.6 Hz, 1H), 4.37 (s, 3H), 3.88 (d, J=7.6 Hz, 1H), 2.08-2.05 (m, 2H), 1.99-1.92 (m, 1H), 1.49-1.28 (m, 7H). LC-MS m/z: 352.1 [M+H]⁺. HPLC Purity (214 nm): 98.74%; t_{R}=8.56 min.

### Example 112—6-Chloro-N-iso-pentyl-4-(4-methylpiperazin-1-yl)-1H-benzo[d]imidazole-1-carboxamide

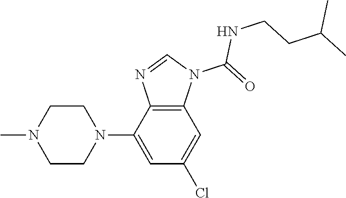 [SMILES: CC(C)CCNC(=O)n1cnc2c(N3CCN(C)CC3)cc(Cl)cc21]

Following general procedure H, 2-bromo-4-chloro-6-nitroaniline (900 mg, 3.59 mmol) and 1-methylpiperazine (430.8 mg, 4.3 mmol) afforded 4-chloro-2-(4-methylpiperazin-1-yl)-6-nitroaniline (280 mg, 96%) as a yellow oil. LC-MS m/z: 271.7 [M+H]⁺. Purity (214 nm): 96%; t_{R}=1.71 min.

Following general procedure F, 4-chloro-2-(4-methylpiperazin-1-yl)-6-nitroaniline (280 mg, 1.04 mmol) afforded 5-chloro-3-(4-methylpiperazin-1-yl)benzene-1,2-diamine (180 mg, 94%) as a yellow oil. LC-MS m/z: 241.6 [M+H]⁺, t_{R}=1.99 min.

Following general procedure G (method B), 5-chloro-3-(4-methylpiperazin-1-yl)benzene-1,2-diamine (180 mg, 0.75 mmol) afforded crude 6-chloro-4-(4-methylpiperazin-1-yl)-1H-benzo[d]imidazole (130 mg, 94%) as a yellow oil. LC-MS m/z: 251.3 [M+H]⁺. Purity (214 nm): 94%; t_{R}=1.48 min.

Following general procedure C, 6-chloro-4-(4-methylpiperazin-1-yl)-1H-benzo[d]imidazole (130 mg, 0.52 mmol) and 3-methylbutan-1-amine (45.2 mg, 0.52 mmol) afforded the title compound (10.2 mg, 5.9%) as a yellow solid. ¹H NMR (400 MHz, CDCl₃) δ 8.43 (s, 1H), 7.51 (d, J=1.6 Hz, 1H), 6.62 (d, J=1.6 Hz, 1H), 6.42 (s, 1H), 3.73 (s, 4H), 3.51 (dd, J=14.9, 5.8 Hz, 2H), 3.07 (s, 4H), 2.64 (d, J=14.2 Hz, 3H), 1.72 (td, J=13.2, 6.6 Hz, 1H), 1.61 (dd, J=14.8, 7.1 Hz, 2H), 0.99 (d, J=6.5 Hz, 6H). LC-MS m/z: 346.3 [M+H]⁺. HPLC Purity (214 nm): 96%: t_{R}=8.66 min.

### Example 113—2-Ethoxy-N-iso-pentyl-3H-imidazo[4,5-b]pyridine-3-carboxamide

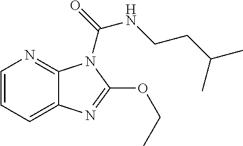 [SMILES: CCOc1nc2cccnc2n1C(=O)NCCC(C)C]

Following general procedure C, 2-ethoxy-1H-imidazo[4,5-b]pyridine (240 mg, 1.47 mmol) and 3-methylbutan-1-amine (128 mg, 1.47 mmol) afforded the title compound (79.3 mg, 19.5%) as a white solid. ¹H NMR (400 MHz, CDCl₃) δ 8.43-8.37 (m, 2H), 7.16 (dd, J=7.8, 5.2 Hz, 1H), 7.00 (s, 1H), 4.85 (q, J=7.1 Hz, 2H), 3.53-3.44 (m, 2H), 1.71 (septet, J=6.4 Hz, 1H), 1.60 (t, J=7.2 Hz, 3H), 1.60-1.51 (m, 2H), 0.99 (d, J=6.4 Hz, 6H). LC-MS m/z: 277.1 [M+H]⁺. HPLC Purity (214 nm): 99%; t_{R}=7.21 min.

### Example 114—2-Ethoxy-N-iso-pentyl-1H-imidazo[4,5-b]pyridine-1-carboxamide

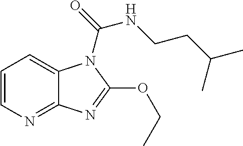 [SMILES: CCOc1nc2ncccc2n1C(=O)NCCC(C)C]

Following general procedure C, 2-ethoxy-1H-imidazo[4,5-b]pyridine (570 mg, 3.49 mmol) and 3-methylbutan-1-amine (304 mg, 3.49 mmol) afforded the title compound (17.8 mg, 1.8%) as a colorless oil. ¹H NMR (400 MHz, CDCl₃) δ 9.53 (s, 1H), 8.13 (dd, J=5.1, 1.4 Hz, 1H), 7.78 (dd, J=7.9, 1.4 Hz, 1H), 7.22 (dd, J=7.9, 5.1 Hz, 1H), 4.71 (q, J=7.1 Hz, 2H), 3.54-3.46 (m, 2H), 1.74 (septet, J=7.2, 1H), 1.64-1.58 (m, 2H), 1.57 (t, J=7.2, 3H), 0.97 (d, J=6.6 Hz, 6H). LC-MS m/z: 277.1 [M+H]⁺. HPLC Purity (214 nm): 100%; t_{R}=9.18 min.

### Example 115—N-iso-Pentyl-2,4-dimethoxy-1H-benzo[d]imidazole-1-carboxamide

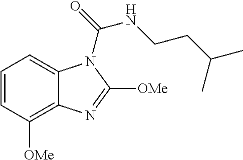 [SMILES: COc1cccc2c1nc(OC)n2C(=O)NCCC(C)C]

A solution of 3-methoxybenzene-1,2-diamine (1.0 g, 7 mmol) in C(OMe)₄ (25 mL) was stirred at RT overnight. The resulting mixture was quenched with water (50 mL), extracted with DCM (50 mL×3) and concentrated in vacuo to give 2,4-dimethoxy-1H-benzo[d]imidazole (450 mg, 35.2%) as a brown solid. LC-MS m/z: 179.0 [M+H]⁺. HPLC Purity (254 nm): 100%; t_{R}=1.33 min.

Following general procedure C, 2,4-dimethoxy-1H-benzo[d]imidazole (150 mg, 1 mmol) and 4-methylpentan-1-amine (85 mg, 1 mmol) afforded the title compound (48.2 mg, 19.6%) as a white solid. ¹H NMR (400 MHz, CDCl₃) δ 7.80 (dd, J=8.0, 0.4 Hz, 1H), 7.16 (t, J=8.2 Hz, 1H), 6.95 (s, 1H), 6.76 (d, J=8.0 Hz, 1H), 4.35 (s, 3H), 4.00 (s, 3H), 3.49-3.42 (m, 2H), 1.69 (septet, J=7.2 Hz, 1H), 1.56-1.49 (m, 2H), 0.97 (d, J=6.6 Hz, 6H). LC-MS m/z: 292.0 [M+H]⁺. HPLC Purity (214 nm): 100%; t_{R}=9.59 min.

### Example 116—N-(2-Cyclopropylethyl)-2-ethoxy-4-methoxy-1H-benzo[d]imidazole-1-carboxamide

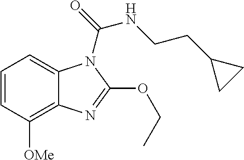 [SMILES: CCOc1nc2c(OC)cccc2n1C(=O)NCCC1CC1]

Following general procedure C, 2-ethoxy-4-methoxy-1H-benzo[d]imidazole (200 mg, 1 mmol) and 2-cyclopropylethanamine (85 mg, 1 mmol) afforded the title compound (23.2 mg, 7.3%) as a white solid. ¹H NMR (400 MHz, CDCl₃) δ 7.80 (dd, J=8.3, 0.7 Hz, 1H), 7.21 (s, 1H), 7.15 (t, J=8.2 Hz, 1H), 6.76 (d, J=8.2 Hz, 1H), 4.81 (q, J=7.1 Hz, 2H), 3.99 (s, 3H), 3.54 (dd, J=12.4, 6.8 Hz, 2H), 1.54 (t, J=7.2 Hz, 5H), 0.80-0.71 (m, 1H), 0.56-0.46 (m, 2H), 0.16-0.12 (m, 2H). LC-MS m/z: 304.2 [M+H]⁺. HPLC Purity (214 nm): 98.52%; t_{R}=9.14 min.

### Example 117—2,4-Dimethoxy-N-(2-methylbutyl)-1H-benzo[d]imidazole-1-carboxamide

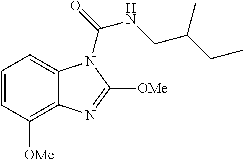 [SMILES: CCC(C)CNC(=O)n1c(OC)nc2c(OC)cccc21]

Following general procedure C, 2,4-dimethoxy-1H-benzo[d]imidazole (150 mg, 0.84 mmol) and 2-methylbutan-1-amine (73 mg, 0.84 mmol) afforded the title compound (68.8 mg, 23.6%) as a white solid. ¹H NMR (400 MHz, CDCl₃) δ 7.80 (d, J=8.2 Hz, 1H), 7.17 (t, J=8.2 Hz, 1H), 7.03 (s, 1H), 6.77 (d, J=8.1 Hz, 1H), 4.37 (s, 3H), 4.00 (s, 3H), 3.46-3.38 (m, 1H), 3.33-3.24 (m, 1H), 1.79-1.71 (m, 1H), 1.54-1.43 (m, 1H), 1.33-1.20 (m, 1H), 1.03-0.98 (m, 6H). LC-MS m/z: 292.0 [M+H]⁺. HPLC Purity (214 nm): 100%; t_{R}=9.59 min.

### Example 118—N-iso-Pentyl-4-morpholino-1H-benzo[d]imidazole-1-carboxamide

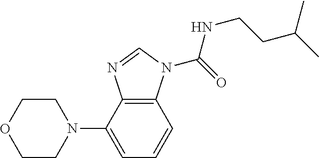 [SMILES: CC(C)CCNC(=O)n1cnc2c(N3CCOCC3)cccc21]

A solution of 4-bromo-1H-benzo[d]imidazole (1.0 g, 5.1 mmol), SEM-Cl (1.0 g, 6.1 mmol) and Et₃N in DCM (10 mL) was stirred at RT overnight. The mixture was concentrated and purified by SGC (DCM/IMEOH=50:1) to give 4-bromo-1-((2-(trimethylsilyl)ethoxy)methyl)-1H-benzo[d]imidazole (800 mg, 48%) as a yellow oil. LC-MS m/z: 328 [M+H]⁺. Purity (214 nm): 70%; t_{R}=2.25 min.

Following general procedure H, 4-bromo-1-((2-(trimethylsilyl)ethoxy)methyl)-1H-benzo[d]imidazole (800 mg, 2.4 mmol) and morpholine (313 mg, 3.6 mmol) afforded 4-morpholino-1-((2-(trimethylsilyl)ethoxy)methyl)-1H-benzo[d]imidazole (700 mg, 87%) as a yellow oil. LC-MS m/z: 334 [M+H]⁺. Purity (214 nm): 78%; t_{R}=2.14 min.

A solution of 4-morpholino-1-((2-(trimethylsilyl)ethoxy)methyl)-1H-benzo[d]imidazole (700 mg, 2.1 mmol) in TBAF (1.0 M in THF, 5 ml) was stirred at 60° C. overnight. The mixture was concentrated and the residue was purified by SGC (DCM/MEOH=50:1) to give 4-morpholino-1H-benzo[d]imidazole (200 mg, 48%) as a yellow oil. LC-MS m/z: 204[M+H]⁺. Purity (214 nm): 96%; t_{R}=1.38 min.

Following general procedure C, 4-morpholino-1H-benzo[d]imidazole (200 mg, 0.98 mmol) and 3-methylbutan-1-amine (86 mg, 0.98 mmol) afforded the title compound (78.6 mg 25.6%) as a white solid. ¹H NMR (400 MHz, CDCl₃) δ 8.49 (d, J=4.2 Hz, 1H), 7.4 (s, 2H), 6.7 (m, 1H), 5.7 (dd, J=17.2, 7.9 Hz, 1H), 3.96 (m, 4H), 3.63 (m, 6H), 1.45-1.23 (m, 3H), 0.95 (dd, J=10.0, 6.6 Hz, 6H). LC-MS m/z: 317 [M+H]⁺. HPLC Purity (214 nm): 97%; t_{R}=7.73 min.

### Example 119—N-iso-Pentyl-4-(4-methylpiperazin-1-yl)-1H-benzo[d]imidazole-1-carboxamide

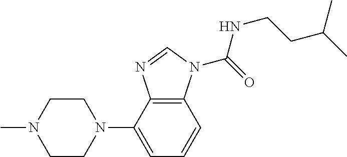 [SMILES: CC(C)CCNC(=O)n1cnc2c(N3CCN(C)CC3)cccc21]

Following general procedure C, 4-(4-methylpiperazin-1-yl)-1H-benzo[d]imidazole (108 mg, 0.5 mmol) and 3-methylbutan-1-amine (54.0 mg, 0.6 mmol) afforded the title compound (27.5 mg, 16.7%) as a white solid. ¹H NMR (400 MHz, CDCl₃) δ 8.56 (s, 1H), 8.53 (s, 1H), 7.59 (d, J=8.2 Hz, 1H), 7.32 (t, J=5.1 Hz, 1H), 7.24 (d, J=8.1 Hz, 1H), 6.70 (d, J=7.9 Hz, 1H), 3.67 (s, 4H), 3.48 (dd, J=14.5, 6.0 Hz, 2H), 3.17 (s, 4H), 2.62 (s, 3H), 1.85-1.64 (m, 1H), 1.58 (dd, J=14.8, 7.1 Hz, 2H), 0.94 (dd, J=19.9, 6.6 Hz, 6H). LC-MS m/z: 330.3 [M+H]⁺. HPLC Purity (214 nm): 100%; t_{R}=4.82 min.

### Example 120—2-(2-(Dimethylamino)ethoxy)-N-iso-pentyl-1H-benzo[d]imidazole-1-carboxamide

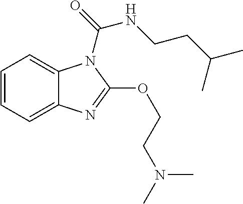 [SMILES: CC(C)CCNC(=O)n1c(OCCN(C)C)nc2ccccc21]

A mixture of 2-chloro-1-((2-(trimethylsilyl)ethoxy)methyl)-1H-benzo[d]imidazole (850 mg, 3.0 mmol), 2-(dimethylamino)ethan-1-ol (347 mg, 3.9 mmol) and t-BuONa (576 mg, 6.0 mmol) in DMF (10 mL) was stirred at 60° C. for 5 h under N₂. The reaction was cooled and concentrated in vacuo to give a residue which was purified by silica gel column chromatography (DCM/MeOH=20/1) to give N,N-dimethyl-2-((1-((2-(trimethylsilyl)ethoxy)methyl)-1H-benzo[d]imidazol-2-yl)oxy)ethan-1-amine (1000 mg, 99%) as a yellow oil. LC-MS m/z: 336.2 [M+H]⁺. HPLC Purity (214 nm): 910%; t_{R}=2.19 min.

To a mixture of N,N-dimethyl-2-((1-((2-(trimethylsilyl)ethoxy)methyl)-1H-benzo[d]imidazol-2-yl)oxy)ethan-1-amine (1000 mg, 3.0 mmol) in THF (5 mL) was added TBAF (15 mL, 1 N in THF). The mixture was stirred at 60° C. for 15 h and concentrated in vacuo to give a residue which was purified by silica gel column chromatography (DCM/MeOH=6/1) to give 2-((1H-benzo[d]imidazol-2-yl)oxy)-N,N-dimethylethane-1-amine (390 mg, crude) as a yellow oil. LC-MS m/z: 206.1 [M+H]⁺. HPLC Purity (214 nm): 89%; t_{R}=1.613 min.

Following general procedure C, 2-((1H-benzo[d]imidazol-2-yl)oxy)-N,N-dimethylethane-1-amine (205 mg, 1.0 mmol) and 3-methylbutan-1-amine (87 mg, 1.0 mmol) afforded the title compound (55.0 mg, 17.3%) as a white solid. ¹H NMR (400 MHz, CDCl₃) δ 8.19-8.15 (m, 1H), 7.63 (brs, 1H), 7.52-7.48 (m, 1H), 7.27-7.23 (m, 2H), 4.73 (t, J=5.2, 2H), 3.49-3.42 (m, 2H), 2.77 (t, J=5.6 Hz, 2H), 2.34 (s, 6H), 1.77-1.70 (m, 1H), 1.56 (q, J=7.2 Hz, 2H), 0.99 (d, J=6.6 Hz, 6H). LC-MS m/z: 319.2 [M+H]⁺. HPLC Purity (214 nm): 100%; t_{R}=9.95 min.

### Example 121—N-(2-Cyclopropylethyl)-4-methoxy-1H-benzo[d]imidazole-1-carboxamide

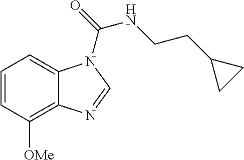 [SMILES: COc1cccc2c1ncn2C(=O)NCCC1CC1]

Following general procedure C, 4-methoxy-1H-benzo[d]imidazole (150 mg, 1.01 mmol) and 2-cyclopropylethan-1-amine (148 mg, 1.22 mmol) afforded the title compound (37.50 mg, 14%) as a white solid. ¹H NMR (400 MHz, CDCl₃) δ 8.35 (s, 1H), 7.41 (d, J=8.0 Hz, 1H), 7.32 (t, J=8.1 Hz, 1H), 6.80 (d, J=7.7 Hz, 1H), 6.11 (brs, 1H), 4.03 (s, 3H), 3.61 (dd, J=12.6, 6.9 Hz, 2H), 1.63-1.57 (m, 2H), 0.79-0.72 (m, 1H), 0.58-0.50 (m, 2H), 0.17-0.13 (m, 2H). LC-MS m/z: 260.7 [M+H]⁺. HPLC Purity (214 nm): 98.36%; t_{R}=7.94 min.

### Example 122—2-Methoxy-N-(4-phenylbutyl)-6-(pyridin-3-yl)-3H-imidazo[4,5-b]pyridine-3-carboxamide

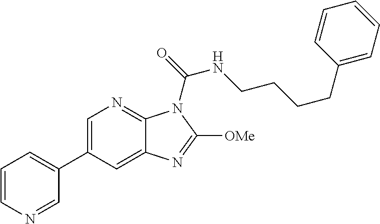 [SMILES: COc1nc2cc(-c3cccnc3)cnc2n1C(=O)NCCCCc1ccccc1]

To a solution of 5-bromopyridine-2,3-diamine (10.0 g, 53.19 mmol) in EtOH (150 mL) was added CS₂ (40.0 g, 532.0 mmol) at RT and the mixture was stirred overnight at 70° C. The reaction mixture was filtered and concentrated in vacuo to give crude 6-bromo-3H-imidazo[4,5-b]pyridine-2-thiol (8.0 g, 79.7%) as a yellow solid. LC-MS m/z: 231.1 [M+H]⁺, t_{R}=1.13 min.

To a mixture of 6-bromo-3H-imidazo[4,5-b]pyridine-2-thiol (8.0 g, 34.78 mmol) and K₂CO₃ (14.4 g, 100.0 mmol) in acetone (100 mL) was added MeI (4.3 g, 31.30 mmol) at RT and the reaction mixture was stirred for 2 h and then concentrated to give a residue which was purified by Prep-HPLC to give 6-bromo-2-(methylthio)-3H-imidazo[4,5-b]pyridine (8.0 g, 96.4%) as a yellow solid. MS m/z: 245.1 [M+H]⁺, t_{R}=1.71 min.

To a solution of 6-bromo-2-(methylthio)-3H-imidazo[4,5-b]pyridine (8.0 g, 32.78 mmol) in DCM (350 mL) was added m-CPMA (22.6 g, 131 mmol) and the reaction mixture was stirred overnight at RT. The resultant solid was filtered, washed with DCM and dried under vacuum to give 6-bromo-2-(methylsulfonyl)-3H-imidazo[4,5-b]pyridine (10.0 g, 98.32%) as a white solid. LC-MS m/z: 279.0 [M+H]⁺, t_{R}=1.86 min.

To a flask contained 6-bromo-2-(methylsulfonyl)-3H-imidazo[4,5-b]pyridine (12.0 g, 43.16 mmol) was added MaOMe in MeOH (150 mL) at RT and the reaction mixture was stirred overnight at 65° C. The mixture cooled, filtered and concentrated in to give crude 6-bromo-2-methoxy-3H-imidazo[4,5-b]pyridine (8.0 g, 81.3%) as a white solid. LC-MS m/z: 231.1 [M+H]⁺, t_{R}=1.13 min.

Following general procedure A, 6-bromo-2-methoxy-3H-imidazo[4,5-b]pyridine (800.0 mg, 3.50 mmol) and pyridin-3-ylboronic acid (516.6 mg, 4.20 mmol) afforded 2-methoxy-6-(pyridin-3-yl)-3H-imidazo[4,5-b]pyridine (150 mg, 19.0%) as a white solid. LC-MS m/z: 227.0 [M+H]⁺. Purity (214 nm): 100.0%; t_{R}=1.28 min.

Following general procedure C, 2-methoxy-6-(pyridin-3-yl)-3H-imidazo[4,5-b]pyridine (100 mg, 0.44 mmol) and 4-phenylbutan-1-amine (100.0 mg, 0.67 mmol) afforded the title compound (18.9 mg, 18.9%) as a white solid. ¹H NMR (400 MHz, CDCl₃) δ 9.43 (s, 1H), 8.87 (d, J=1.8 Hz, 1H), 8.67 (dd, J=4.8, 1.3 Hz, 1H), 8.34 (d, J=1.9 Hz, 1H), 7.99 (d, J=1.9 Hz, 1H), 7.89 (d, J=7.9 Hz, 1H), 7.44 (dd, J=7.8, 5.0 Hz, 1H), 7.29-7.26 (m, 2H), 7.21-7.15 (m, 3H), 4.32 (s, 3H), 3.53 (q, J=6.3 Hz, 2H), 2.70 (t, J=7.0 Hz, 2H), 1.85-1.67 (m, 4H). LC-MS m/z: 402.0 [M+H]⁺. HPLC Purity (214 nm): 100.0%; t_{R}=6.74 min.

### Example 123—N-iso-Butyl-3-methyl-2-oxo-6-(pyridin-3-yl)-2,3-dihydro-1H-benzo[d]imidazole-1-carboxamide

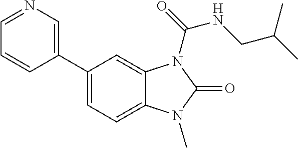 [SMILES: CC(C)CNC(=O)n1c(=O)n(C)c2ccc(-c3cccnc3)cc21]

Following general procedure G (method A), 4-bromobenzene-1,2-diamine (2.0 g, 10.8 mmol) afforded 6-bromo-2-methoxy-1H-benzo[d]imidazole (2.0 g, 82%) as a white solid. ¹H NMR (400 MHz, DMSO) δ 7.48 (d, J=1.6 Hz 1H), 7.25 (d, J=8.4 Hz 1H), 7.17 (dd, J=6.4, 2.0 1H), 4.05 (s, 3H). LC-MS m/z: 227.0 [M+H]⁺. Purity (214 nm): 100%; t_{R}=0.86 min.

Following general procedure A, 6-bromo-2-methoxy-1H-benzo[d]imidazole (2.0 g, 8.8 mmol) and pyridin-5-ylboronic acid (1.64 g, 13.3 mmol) afforded 2-methoxy-6-(pyrimidin-5-yl)-1H-benzo[d]imidazole (1.0 g, 50%) as a white solid. LC-MS m/z: 227.1 [M+H]⁺.

Following general procedure C, 2-methoxy-6-(pyrimidin-5-yl)-1H-benzo[d]-imidazole (226 mg, 1.0 mmol) and 2-methylpropan-1-amine (73 mg, 1 mmol) afforded the title compound (1.1 mg, 0.34%) as a white solid. ¹H NMR (400 MHz, CDCl₃) δ 8.82 (s, 1H), 8.75 (s, 1H), 8.56 (s, 1H), 8.27 (d, J=8.0 Hz, 1H), 7.87 (d, J=8.0 Hz, 1H), 7.39-7.36 (m, 2H), 7.18 (s, 1H), 3.58 (s, 3H), 3.37 (t, J=6.0 Hz, 2H), 2.06 (sep, J=6.4 Hz, 1H), 1.16 (d, J=6.4 Hz, 6H). LC-MS m/z: 325.7 [M+H]⁺. HPLC Purity (214 nm): 100.0%; t_{R}=6.59 min.

### Example 124—2-Oxo-N-phenethyl-1H-imidazo[4,5-b]pyridine-3(2H)-carboxamide

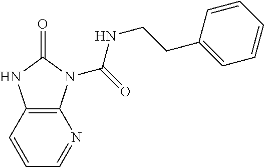 [SMILES: O=C(NCCc1ccccc1)n1c(=O)[nH]c2cccnc21]

A suspension of 5-bromo-1H-imidazo[4,5-b]pyridin-2(3H)-one (400 mg, 4.7 mmol) and Pd/C (100 mg, w/w 10%) in MeOH (100 ml) was stirred for 16 h at RT, then filtered and concentrated to give 1H-imidazo[4,5-b]pyridin-2(3H)-one (300 mg) as a yellow oil which was used directly in the next step. LC-MS m/z: 136.1 [M+H]⁺. Purity (214 nm): 94%; t_{R}=0.89 min

Following general procedure B, 1H-imidazo[4,5-b]pyridin-2(3H)-one (300 mg, 2.2 mmol), and phenethyl isocyanate (650 mg, 4.4 mmol) afforded the title compound (74.8 mg, 11.9%) as a white solid. ¹H NMR (400 MHz, CDCl₃) δ 8.58 (bs, 1H), 8.37 (dd, J=8.0, 1.2 Hz, 1H), 8.17 (dd, J=5.2, 1.2 Hz, 1H), 7.37-7.31 (m, 2H), 7.26-7.21 (m, 4H), 7.14 (dd, J=8.0, 5.3 Hz, 1H), 3.70 (dd, J=13.2, 7.1 Hz, 2H), 2.96 (t, J=7.2 Hz, 2H). LC-MS m/z: 283.3 [M+H]⁺. HPLC Purity (214 nm): >96%; t_{R}=7.81 min.

### Example 125—N-iso-Butyl-2-(methylthio)-5-(pyridin-3-yl)-1H-benzo[d]imidazole-1-carboxamide

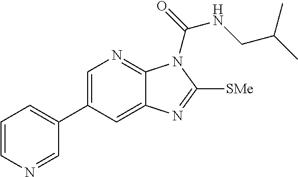 [SMILES: CSc1nc2cc(-c3cccnc3)cnc2n1C(=O)NCC(C)C]

A suspension of 6-bromo-2-(methylthio)-3H-imidazo[4,5-b]pyridine (500 mg, 2.0 mmol) and pyridin-5-ylboronic acid (200 mg, 3.5 mmol) afforded 2-(methylthio)-6-(pyridin-3-yl)-3H-imidazo[4,5-b]pyridine (60 mg, 98%) as an off white solid. LC-MS m/z: 242.1 [M+H]⁺, t_{R}=1.36 min.

Following general procedure B, 2-(methylthio)-6-(pyridin-3-yl)-3H-imidazo[4,5-b]pyridine (60 mg, 0.2 mmol) and 2-methylpropan-1-amine (158 mg, 1.6 mmol) afforded the title compound (1.8 mg, 2.1%) as a yellow solid. ¹H NMR (400 MHz, CDCl₃) δ 9.50 (brs, 1H), 8.82 (d, J=2.0 Hz, 1H), 8.60 (dd, J=4.8 Hz, 1.2 Hz, 1H), 8.36 (d, J=2.0 Hz, 1H), 8.05 (d, J=2.0 Hz, 1H), 7.86-7.83 (m, 1H), 7.37 (dd, J=7.6 Hz, 4.8 Hz, 1H), 3.30 (t, J=6.4 Hz, 2H), 2.65 (s, 3H), 1.98-1.91 (m, 1H), 0.98 (d, J=6.8 Hz, 6H). LC-MS m/z: 342.0 [M+H]⁺. HPLC Purity (214 nm): 100%; t_{R}=6.92 min.

### Examples 126a and 126b—N-iso-Butyl-5-(thiazol-5-yl)-1H-benzo[d]imidazole-1-carboxamide and N-iso-butyl-6-(thiazol-5-yl)-1H-benzo[d]imidazole-1-carboxamide

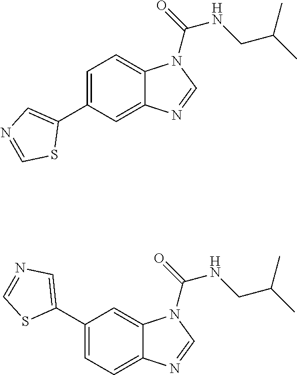 [SMILES: CC(C)CNC(=O)n1cnc2cc(-c3cncs3)ccc21 ; CC(C)CNC(=O)n1cnc2ccc(-c3cncs3)cc21]

Following general procedure A, 5-bromo-1-(4-methoxybenzyl)-1H-benzo[d]imidazole (1.8 g, 3.8 mmol) and thiazol-5-ylboronic acid (1.2 g, 3.8 mmol) afforded 5-(1-(4-methoxybenzyl)-1H-benzo[d]imidazol-5-yl)thiazole (700 mg, 38%) as a black oil. LC-MS m/z: 322.0 [M+H]⁺. Purity (254 nm): 91.1%; t_{R}=1.1 min.

A solution of 5-(1-(4-methoxybenzyl)-1H-benzo[d]imidazol-5-yl)thiazole (700 mg, 2.2 mmol) in TFA (15 mL) was stirred at 135° C. for 12 h under microwave. The mixture was concentrated and purified by silica gel column chromatography (DCM/MeOH=5/1) to give 5-(1H-benzo[d]imidazol-5-yl)thiazole (500 mg) as a yellow solid. LC-MS m/z: 202.01 [M+H]⁺. Purity (254 nm): 98.15%; t_{R}=1.54 min.

Following general procedure C, 5-(1H-benzo[d]imidazol-5-yl)thiazole (230 mg, 1.14 mmol) and 2-methylpropan-1-amine (83 mg, 1.14 mmol) afforded N-iso-butyl-5-(thiazol-5-yl)-1H-benzo[d]imidazole-1-carboxamide (6.4 mg, 1.9%) as a white solid and N-iso-butyl-6-(thiazol-5-yl)-1H-benzo[d]imidazole-1-carboxamide (2.0 mg, 0.6%) as a brown solid.

N-iso-butyl-5-(thiazol-5-yl)-1H-benzo[d]imidazole-1-carboxamide: ¹H NMR (400 MHz, CDCl₃) δ 8.80 (s, 1H), 8.46 (s, 1H), 8.15 (s, 1H), 8.06 (s, 1H), 7.91 (d, J=8.6 Hz, 1H), 7.66 (d, J=8.6 Hz, 1H), 5.81 (bs, 1H), 3.39 (t, J=6.6 Hz, 2H), 2.03 (sep, J=6.8 Hz, 1H), 1.08 (d, J=6.7 Hz, 6H). LC-MS m/z: 301.0 [M+H]⁺. HPLC Purity (214 nm): 95.26%; t_{R}=7.06 min.

N-iso-butyl-6-(thiazol-5-yl)-1H-benzo[d]imidazole-1-carboxamide: ¹H NMR (400 MHz, CDCl₃) δ 8.81 (s, 1H), 8.41 (s, 1H), 8.16 (d, J=9.6 Hz, 2H), 7.87 (d, J=8.5 Hz, 1H), 7.64 (d, J=8.3 Hz, 1H), 5.87 (s, 1H), 3.41 (t, J=6.3 Hz, 2H), 2.03 (sep, J=6.8 Hz, 1H), 1.09 (d, J=6.6 Hz, 6H). LC-MS m/z: 301.0 [M+H]⁺. HPLC Purity (214 nm): 97.77%; t_{R}=7.26 min.

### Example 127—1-Ethyl-2-oxo-N-phenethyl-1H-imidazo[4,5-b]pyridine-3(2H)-carboxamide

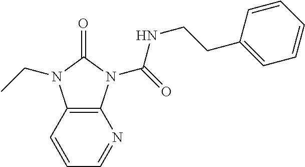 [SMILES: CCn1c(=O)n(C(=O)NCCc2ccccc2)c2ncccc21]

A solution of 3-fluoro-2-nitropyridine (600 mg, 4.23 mmol) and ethanamine (228 mg, 5.07 mmol) in CH₃CN (10 mL) was stirred at 65° C. overnight. The mixture was concentrated and purified by SGC (DCM/MeOH=50:1) to give N-ethyl-2-nitropyridin-3-amine (600 mg, 85.0%) as a yellow oil. LC-MS m/z: 168 [M+H]⁺. Purity (214 nm): 98%; t_{R}=1.70 min.

Following general procedure F, N-ethyl-2-nitropyridin-3-amine (600 mg, 3.6 mmol) afforded N-3-ethylpyridine-2,3-diamine (500 mg, 98%) as a yellow oil. LC-MS m/z: 138 [M+H]⁺. Purity (214 nm): 93%; t_{R}=1.58 min.

A solution of N-3-ethylpyridine-2,3-diamine (500 mg, 3.64 mmol), CDI (775 mg, 4.7 mmol) and Et₃N (805 mg, 7.9 mmol) in THF (50 mL) was stirred at RT for 3 h, then cooled, concentrated and purified by silica gel column chromatography (DCM/MeOH=10:1) to give 1-ethyl-1H-imidazo[4,5-b]pyridin-2(3H)-one (300 mg, 50%) as a brown oil. LC-MS m/z: 164 [M+H]⁺. Purity (214 nm): 100%; t_{R}=1.47 min.

Following general procedure B, 1-ethyl-1H-imidazo[4,5-b]pyridin-2(3H)-one (150 mg, 0.92 mmol) afforded the title compound (53 mg 18%) as a white solid. ¹H NMR (400 MHz, CDCl₃) δ 8.84 (s, 1H), 8.17 (d, J=5.2 Hz, 1H), 7.38-7.20 (m, 6H), 7.14 (t, J=5.6 Hz, 1H), 3.94 (q, J=7.2 Hz, 2H), 3.71 (dd, J=13.2, 6.7 Hz, 2H), 2.98 (t, J=7.1 Hz, 2H), 1.35 (t, J=7.2 Hz, 3H). LC-MS m/z: 311.0 [M+H]⁺. HPLC Purity (214 nm): 99%; t_{R}=7.30 min.

### Example 128—N-Phenethyl-1H-benzo[d]imidazole-2-d-1-carboxamide

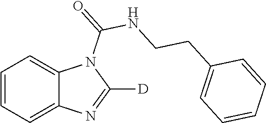 [SMILES: [2H]c1nc2ccccc2n1C(=O)NCCc1ccccc1]

Following general procedure G (method A), benzene-1,2-diamine (500 mg, 4.6 mmol) afforded crude 1H-benzo[d]imidazole-2-d (840 mg) as a yellow oil.

Following general procedure B, 1H-benzo[d]imidazole-2-d (200 mg, 1.7 mmol) and (2-isocyanatoethyl)benzene (296 mg, 2.0 mmol) afforded the title compound (25.0 mg, 5.6%) as a white solid. ¹H NMR (400 MHz, CDCl₃) δ 7.80 (dd, J=6.7, 2.3 Hz, 1H), 7.51 (dd, J=6.6, 2.1 Hz, 1H), 7.41-7.27 (m, 7H), 5.85 (s, 1H), 3.81 (dd, J=12.6, 6.7 Hz, 2H), 3.03 (t, J=6.8 Hz, 2H). LC-MS m/z: 267.0 [M+H]⁺. HPLC Purity (214 nm): 96.97%; t_{R}=8.16 min.

### Example 129—2-Methoxy-N-(3-phenoxypropyl)-1H-imidazo[4,5-h]pyridine-1-carboxamide

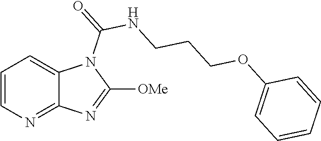 [SMILES: COc1nc2ncccc2n1C(=O)NCCCOc1ccccc1]

Following general procedure C, 2-methoxy-1H-imidazo[4,5-b]pyridine (100 mg, 0.67 mmol) and 3-phenoxypropan-1-amine (152.51 mg, 1.01 mmol) afforded the title compound (3.4 mg, 1.7%) as a yellow solid. ¹H NMR (400 MHz, CDCl₃) δ 9.69 (s, 1H), 8.05 (d, J=5.0 Hz, 1H), 7.81 (d, J=8.1 Hz, 1H), 7.29 (d, J=8.3 Hz, 2H), 7.26-7.22 (m, 1H), 6.94 (dd, J=16.2, 8.1 Hz, 3H), 4.29 (s, 3H), 4.12 (t, J=6.0 Hz, 2H), 3.71 (dd, J=12.6, 6.6 Hz, 2H), 2.19 (p, J=6.4 Hz, 2H). LC-MS m/z: 327.1 [M+H]⁺. HPLC Purity (214 nm): 97.29%; t_{R}=8.72 min.

### Example 130—4-Cyano-N-(4-phenylbutyl)-1H-benzo[d]imidazole-1-carboxamide

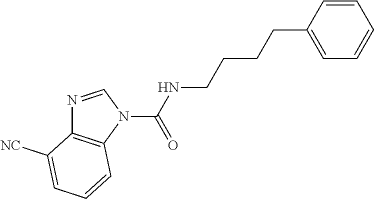 [SMILES: N#Cc1cccc2c1ncn2C(=O)NCCCCc1ccccc1]

Following general procedure C, 1H-benzo[d]imidazole-4-carbonitrile (70.0 mg, 0.5 mmol) and 4-phenylbutan-1-amine (75.0 mg, 0.5 mmol) afforded the title compound (40.2 mg, 25.3%) as a white solid. ¹H NMR (400 MHz, CDCl₃) δ 8.61 (s, 1H), 8.27 (d, J=8.2 Hz, 1H), 7.59 (d, J=7.4 Hz, 1H), 7.40 (t, J=7.8 Hz, 1H), 7.25 (d, J=8.7 Hz, 1H), 7.22 (s, 1H), 7.19-7.14 (m, 3H), 7.04 (s, 1H), 3.51 (bs, 2H), 2.65 (bs, 2H), 1.72 (bs, 4H). LC-MS m/z: 319.3 [M+H]⁺. HPLC Purity (214 nm): 100%; t_{R}=8.41 min.

### Example 131—4-Cyano-N-(3-phenoxypropyl)-1H-benzo[d]imidazole-1-carboxamide

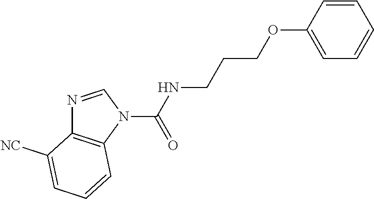 [SMILES: N#Cc1cccc2c1ncn2C(=O)NCCCOc1ccccc1]

Following general procedure C, 1H-benzo[d]imidazole-4-carbonitrile (70.0 mg, 0.5 mmol) and 3-phenoxypropan-1-amine (75.0 mg, 0.5 mmol) afforded the title compound (22.4 mg, 14.0%) as a white solid. ¹H NMR (400 MHz, CDCl₃) δ 8.50 (s, 1H), 8.26 (d, J=8.3 Hz, 1H), 7.69 (d, J=7.6 Hz, 1H), 7.43 (t, J=8.0 Hz, 1H), 7.33-7.27 (m, 2H), 7.00 (t, J=7.4 Hz, 1H), 6.88 (d, J=7.9 Hz, 3H), 4.23 (t, J=5.4 Hz, 2H), 3.79 (dd, J=11.8, 5.6 Hz, 2H), 2.42-2.38 (m, 2H). LC-MS m/z: 321.2 [M+H]⁺. HPLC Purity (214 nm): 97.98%; t_{R}=7.85 min.

### Example 132—N-(4-Cyclopropylbutyl)-6-methoxy-4-(piperidin-1-yl)-1H-benzo[d]imidazole-1-carboxamide

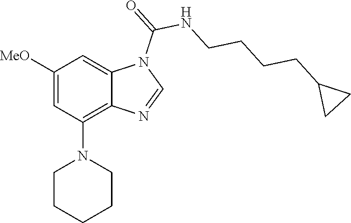 [SMILES: COc1cc(N2CCCCC2)c2ncn(C(=O)NCCCCC3CC3)c2c1]

A mixture of 2-bromo-4-methoxy-6-nitroaniline (984 mg, 4.0 mmol), Zn (1.04 g, 16 mmol) and NH₄Cl (1.0 g, 16 mmol) in EtOH/H₂O (20 mL, 2/1) was stirred at 80° C. for 2 h under N₂. The reaction was cooled, filtered and concentrated in vacuo to give crude 3-bromo-5-methoxybenzene-1,2-diamine (900 mg) as a yellow solid. LC-MS m/z: 217.2 [M+H]⁺. Purity (214 nm): 82%; t_{R}=0.74 min.

Following general procedure G (method B), 3-bromo-5-methoxybenzene-1,2-diamine (864 mg, 4.0 mmol) afforded crude 4-bromo-6-methoxy-1H-benzo[d]imidazole (510 mg) as a yellow solid. LC-MS m/z: 227.1 [M+H]⁺. Purity (214 nm): 59%; t_{R}=0.67 min.

To a solution of 4-bromo-6-methoxy-1H-benzo[d]imidazole (565 mg, 2.5 mmol) in THF (15 mL) was added NaH (200 mg, 5.0 mmol) at 0° C. and the mixture was stirred for 30 min followed by the addition of PMBCl (515 mg, 3.3 mmol). The solution was stirred at 60° C. for 3 h and poured into ice-water (20 mL). The mixture was extracted with EA (50 mL), concentrated and purified by silica gel column chromatography (PE:EA=1:1) to give 4-bromo-6-methoxy-1-(4-methoxybenzyl)-1H-benzo[d]imidazole (700 mg, 97%) as a yellow solid. LC-MS m/z: 347.1 [M+H]⁺. Purity (214 nm): 92.1%; t_{R}=1.76 min.

Following general procedure H, 4-bromo-6-methoxy-1-(4-methoxybenzyl)-1H-benzo[d]imidazole (350 mg, 1 mmol) and morpholine (522 mg, 6 mmol) afforded 6-methoxy-1-(4-methoxybenzyl)-4-(piperidin-1-yl)-1H-benzo[d]imidazole (350 mg, 99%) as a yellow solid. LC-MS m/z: 352.3 [M+H]⁺. Purity (214 nm): 96.1%; t_{R}=1.90 min.

A solution of 6-methoxy-1-(4-methoxybenzyl)-4-(piperidin-1-yl)-1H-benzo[d]-imidazole (350 mg, 1.0 mmol) in TFA (5 mL) was stirred at 135° C. for 6 h under microwave and then concentrated. The residue was purified by silica gel column chromatography (DCM:MeOH=10:1) to give 6-methoxy-4-(piperidin-1-yl)-1H-benzo[d]imidazole (190 mg, 82%) as a yellow solid. LC-MS m/z: 232.2 [M+H]⁺. Purity (214 nm): 92.1%; t_{R}=1.55 min.

Following general procedure C, 6-methoxy-4-(piperidin-1-yl)-1H-benzo[d]imidazole (116 mg, 0.5 mmol) and 4-cyclopropylbutan-1-amine (56.0 mg, 0.5 mmol) afforded the title compound (13.3 mg, 16.6%) as a white solid. ¹H NMR (400 MHz, CDCl₃) δ 8.15 (s, 1H), 6.91 (d, J=2.2 Hz, 1H), 6.40 (s, 1H), 5.71 (s, 1H), 3.85 (s, 3H), 3.68-2.96 (m, 6H), 1.87-1.81 (m, 2H), 1.64 (dd, J=11.3, 5.8 Hz, 2H), 1.58-1.51 (m, 4H), 1.27 (q, J=7.1 Hz, 2H), 0.76-0.65 (m, 1H), 0.42 (ddd, J=8.0, 5.6, 4.2 Hz, 2H), 0.06-0.00 (m, 2H). LC-MS m/z: 371.2 [M+H]⁺. HPLC Purity (214 nm): 97.82%; t_{R}=7.70 min.

### Examples 133a and 133b —2-Methoxy-6-morpholino-N-(4-phenylbutyl)-1H-benzo[d]imidazole-1-carboxamide and 2-methoxy-5-morpholino-N-(4-phenylbutyl)-1H-benzo[d]imidazole-1-carboxamide

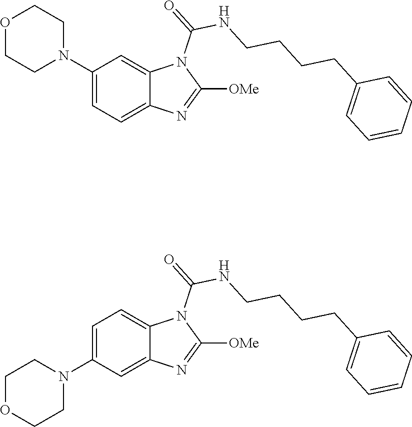 [SMILES: COc1nc2ccc(N3CCOCC3)cc2n1C(=O)NCCCCc1ccccc1 ; COc1nc2cc(N3CCOCC3)ccc2n1C(=O)NCCCCc1ccccc1]

Following general procedure E, 4-fluoro-1,2-dinitrobenzene (2.18 g, 11.7 mmol) and morpholine (1.22 g, 14.0 mmol) afforded crude 4-(3,4-dinitrophenyl)morpholine (2.9 g) as a yellow solid. LC-MS m/z: 254.0 [M+H]⁺. Purity (214 nm): 70.67%; t_{R}=1.88 min.

Following general procedure F, 4-(3,4-dinitrophenyl)morpholine (700 mg, 2.7 mmol) afforded 4-morpholinobenzene-1,2-diamine (650 mg, 99%) as a yellow solid. LC-MS m/z: 194.2 [M+H]⁺. Purity (214 nm): 86.08%; t_{R}=0.75 min.

Following general procedure G (method A), 4-morpholinobenzene-1,2-diamine (650 mg, 3.4 mmol) afforded 4-(2-methoxy-1H-benzo[d]imidazol-6-yl)morpholine (50 mg, 6.4%) as a yellow solid. LC-MS m/z: 234.2 [M+H]⁺. Purity (214 nm): 97.41%; t_{R}=0.51 min.

Following general procedure B, 4-(2-methoxy-1H-benzo[d]imidazol-6-yl)morpholine (50 mg, 0.21 mmol) and 4-phenylbutan-1-amine (31.3 mg, 0.21 mmol) afforded 2-methoxy-6-morpholino-N-(4-phenylbutyl)-1H-benzo[d]imidazole-1-carboxamide (29.5 mg, 8.4%) and 2-methoxy-5-morpholino-N-(4-phenylbutyl)-1H-benzo[d]imidazole-1-carboxamide (69.6 mg, 19.8%) as white solids.

2-methoxy-6-morpholino-N-(4-phenylbutyl)-1H-benzo[d]imidazole-1-carboxamide: ¹H NMR (400 MHz, CDCl₃) δ 8.05 (d, J=8.9 Hz, 1H), 7.33-7.29 (m, 2H), 7.24-7.19 (m, 3H), 7.08 (d, J=2.3 Hz, 1H), 6.91 (dd, J=8.9, 2.4 Hz, 2H), 4.31 (s, 3H), 3.96-3.92 (m, 4H), 3.47 (q, J=6.0 Hz, 2H), 3.21-3.13 (m, 4H), 2.70 (t, J=7.3 Hz, 2H), 1.79-1.73 (m, 4H). LC-MS m/z: 409.0 [M+H]⁺. HPLC Purity (214 nm): 100.0%; t_{R}=7.67 min.

2-methoxy-5-morpholino-N-(4-phenylbutyl)-1H-benzo[d]imidazole-1-carboxamide: ¹H NMR (400 MHz, CDCl₃) δ 7.85 (d, J=2.4 Hz, 1H), 7.41 (d, J=8.7 Hz, 1H), 7.35-7.28 (m, 2H), 7.23-7.19 (m, 3H), 6.99 (s, 1H), 6.93 (dd, J=8.7, 2.4 Hz, 1H), 4.29 (s, 3H), 3.94-3.84 (m, 4H), 3.47 (q, J=6.0 Hz, 2H), 3.23-3.19 (m, 4H), 2.70 (t, J=7.3 Hz, 2H), 1.77-1.67 (m, 4H). LC-MS m/z: 409.0 [M+H]⁺. HPLC Purity (214 nm): 100.0%; t_{R}=7.66 min.

### Examples 134a and 134b —2-Methoxy-6-morpholino-N-(3-phenylpropyl)-1H-benzo[d]imidazole-1-carboxamide and 2-methoxy-5-morpholino-N-(3-phenylpropyl)-1H-benzo[d]imidazole-1-carboxamide

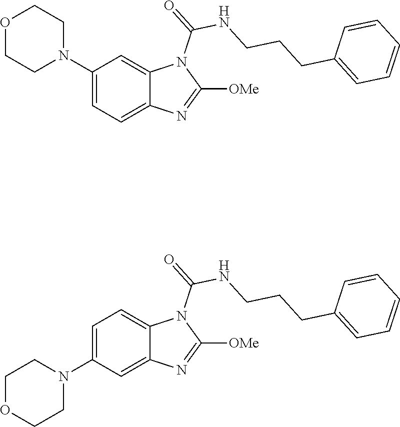 [SMILES: COc1nc2ccc(N3CCOCC3)cc2n1C(=O)NCCCc1ccccc1 ; COc1nc2cc(N3CCOCC3)ccc2n1C(=O)NCCCc1ccccc1]

Following general procedure C, 4-(2-methoxy-1H-benzo[d]imidazol-6-yl)morpholine (100 mg, 0.43 mmol) and 3-phenylpropan-1-amine (58 mg, 0.43 mmol) afforded 2-methoxy-6-morpholino-N-(3-phenylpropyl)-1H-benzo[d]imidazole-1-carboxamide (9.3 mg, 5.5%) and 2-methoxy-5-morpholino-N-(3-phenylpropyl)-1H-benzo[d]imidazole-1-carboxamide (35 mg, 20.7%) both as white solids.

2-methoxy-6-morpholino-N-(3-phenylpropyl)-1H-benzo[d]imidazole-1-carboxamide ¹H NMR (400 MHz, CDCl₃) δ 8.03 (d, J=8.9 Hz, 1H), 7.34-7.28 (m, 2H), 7.24-7.21 (m, 3H), 7.06 (d, J=2.3 Hz, 1H), 6.89 (dd, J=8.9, 2.4 Hz, 2H), 4.29 (s, 3H), 3.91-3.85 (m, 4H), 3.49 (q, J=6.1 Hz, 2H), 3.20-3.17 (m, 4H), 2.73 (t, J=7.6 Hz, 2H), 2.01 (p, J=7.2 Hz, 2H). LC-MS m/z: 395.0 [M+H]⁺. HPLC Purity (214 nm): 98.94%; t_{R}=8.00 min.

2-methoxy-5-morpholino-N-(3-phenylpropyl)-1H-benzo[d]imidazole-1-carboxamide ¹H NMR (400 MHz, CDCl₃) δ 7.85 (d, J=2.8 Hz, 1H), 7.41 (d, J=8.7 Hz, 1H), 7.38-7.31 (m, 2H), 7.24-7.20 (m, 3H), 7.00 (bs, 1H), 6.93 (dd, J=8.7, 2.4 Hz, 1H), 4.30 (s, 3H), 3.95-3.90 (m, 4H), 3.48 (q, J=6.7 Hz, 2H), 3.21-3.18 (m, 4H), 2.76 (t, J=7.6 Hz, 2H), 2.02 (p, J=7.2 Hz, 2H). LC-MS m/z: 395.0 [M+H]⁺. HPLC Purity (214 nm): 100%; t_{R}=7.29 min.

### Examples 135a and 135b—N-(4-(3-(tert-Butyl)-1H-pyrazol-1-yl)butyl)-2-methoxy-3H-imidazo[4,5-b]pyridine-3-carboxamide and N-(4-(3-(tert-butyl)-1H-pyrazol-1-yl)butyl)-1-methyl-2-oxo-1,2-dihydro-3H-imidazo[4,5-b]pyridine-3-carboxamide

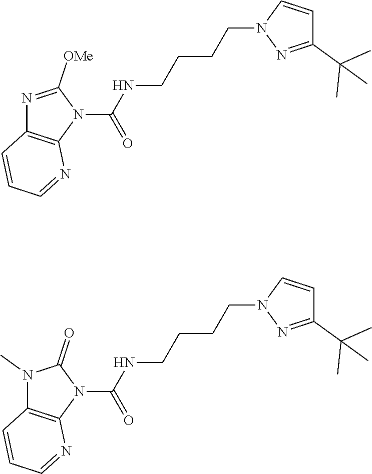

Following general procedure C, 2-methoxy-3H-imidazo[4,5-b]pyridine (100 mg, 0.67 mmol) and 4-(3-(tert-butyl)-1H-pyrazol-1-yl)butan-1-amine (196 mg, 1.00 mmol) afforded N-(4-(3-(tert-butyl)-1H-pyrazol-1-yl)butyl)-2-methoxy-3H-imidazo[4,5-b]pyridine-3-carboxamide (5.0 mg, 2.0%) as a clear oil and N-(4-(3-(tert-butyl)-1H-pyrazol-1-yl)butyl)-1-methyl-2-oxo-1,2-dihydro-3H-imidazo[4,5-b]pyridine-3-carboxamide (2.0 mg, 0.81%) as a yellow solid.

N-(4-(3-(tert-butyl)-1H-pyrazol-1-yl)butyl)-2-methoxy-3H-imidazo[4,5-b]pyridine-3-carboxamide: ¹H NMR (400 MHz, CDCl₃) δ 9.58 (s, 1H), 8.14 (d, J=4.4 Hz, 1H), 7.81 (d, J=8.0 Hz, 1H), 7.30-7.22 (m, 2H), 6.06 (d, J=2.4 Hz, 1H), 4.29 (s, 3H), 4.13 (t, J=7.6 Hz, 2H), 3.48 (q, J=6.8 Hz, 2H), 1.99-1.94 (m, 2H), 1.72-1.65 (m, 2H), 1.29 (s, 9H). LC-MS m/z: 371.0 [M+H]⁺. HPLC Purity (214 nm): 99%; t_{R}=8.57 min.

N-(4-(3-(tert-butyl)-1H-pyrazol-1-yl)butyl)-1-methyl-2-oxo-1,2-dihydro-3H-imidazo[4,5-b]pyridine-3-carboxamide: ¹H NMR (400 MHz, CDCl₃) δ 8.59 (s, 1H), 8.32 (d, J=7.9 Hz, 1H), 8.17 (d, J=5.1 Hz, 1H), 7.26 (s, 1H), 7.11 (dd, J=7.9, 5.3 Hz, 1H), 6.06 (d, J=2.0 Hz, 1H), 4.11 (t, J=7.1 Hz, 2H), 3.51 (s, 3H), 3.43 (q, J=7.1 Hz, 2H), 1.93 (q, J=7.4 Hz, 2H), 1.65-1.55 (m, 2H), 1.25 (s, 9H). LC-MS m/z: 370.2 [M+H]⁺. HPLC Purity (214 nm): 95.5%; t_{R}=8.86 min.

### Example 136—6-Methoxy-4-(1-methylpiperidin-4-yl)-N-(4-phenylbutyl)-1H-benzo[d]imidazole-1-carboxamide

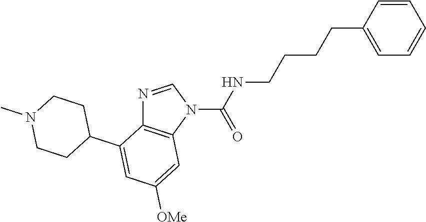 [SMILES: COc1cc(C2CCN(C)CC2)c2ncn(C(=O)NCCCCc3ccccc3)c2c1]

A suspension of 2-bromo-4-methoxy-6-nitrobenzenamine (1.0 g, 4.0 mmol), Zn (1.05 g, 16 mmol) and NH₄Cl (870 mg, 16 mmol) in EtOH/H₂O (10 mL/5 mL) was stirred at 80° C. for 2 h. The mixture was filtered and the residue was concentrated to give crude 3-bromo-5-methoxybenzene-1,2-diamine (1.0 g, 80%) as a yellow oil. LC-MS m/z: 217 [M+H]⁺. Purity (214 nm): 70%; t_{R}=1.73 min.

Following general procedure G (method A), 3-bromo-5-methoxybenzene-1,2-diamine (1.0 g, 4.63 mmol) afforded crude 4-bromo-6-methoxy-1H-benzo[d]imidazole as a yellow oil (700 mg, 86%). LC-MS m/z: 227 [M+H]⁺. Purity (214 nm): 90%; t_{R}=1.40 min.

Following general procedure A, 4-bromo-6-methoxy-1H-benzo[d]imidazole (600 mg, 2.65 mmol) and 1-methyl-4-(4,4,5,5-tetramethyl-1,3,2-dioxaborolan-2-yl)-1,2,3,6-tetrahydro pyridine (691 mg, 3.1 mmol) afforded 6-methoxy-4-(1-methyl-1,2,3,6-tetrahydropyridin-4-yl)-1H-benzo[d]imidazole (400 mg, 62%) as a brown solid. LC-MS m/z: 244 [M+H]⁺. Purity (214 nm): 99%; t_{R}=1.33 min.

A mixture of 6-methoxy-4-(1-methyl-1,2,3,6-tetrahydropyridin-4-yl)-1H-benzo[d]imidazole (400 mg, 1.65 mmol) and PdOH (100 mg) in MeOH (10 mL) was stirred at 50° C. under H₂ overnight. The mixture was filtered, concentrated and purified by silica gel column chromatography (DCM:MeOH=10:1) to give 6-methoxy-4-(1-methylpiperidin-4-yl)-1H-benzo[d]imidazole (400 mg, 98%) as a brown solid. LC-MS m/z: 246 [M+H]⁺. Purity (214 nm): 90%; t_{R}=1.29 min.

Following general procedure C, 6-methoxy-4-(1-methylpiperidin-4-yl)-1H-benzo[d]imidazole (200 mg, 0.8 mmol) and 4-phenylbutan-1-amine (120 mg, 0.8 mmol) afforded the title compound (24.6 mg, 7%) as a white solid. ¹H NMR (400 MHz, CDCl₃) δ 8.61 (s, 1H), 8.43 (bs, 2H), 7.27-7.23 (m, 2H), 7.19-7.17 (m, 2H), 6.75 (d, J=1.7 Hz, 1H), 5.18 (s, 1H), 3.82 (s, 3H), 3.50-3.43 (m, 2H), 3.40-3.29 (m, 3H), 2.78-2.60 (m, 7H), 2.23-2.15 (m, 2H), 2.01-1.98 (m, 2H), 1.75 (bs, 4H). LC-MS m/z: 421.1 [M+H]⁺. Purity (214 nm): 99%; t_{R}=5.56 min.

### Example 137—4-Morpholino-N-(4-phenylbutyl)-1H-benzo[d]imidazole-1-carboxamide

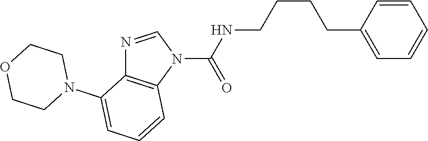 [SMILES: O=C(NCCCCc1ccccc1)n1cnc2c(N3CCOCC3)cccc21]

Following general procedure C, 4-(1H-benzo[d]imidazol-4-yl)morpholine (102 mg, 0.5 mmol) and 4-phenylbutan-1-amine (75.0 mg, 0.5 mmol) afforded the title compound (41.5 mg, 21.9%) as a white solid. ¹H NMR (400 MHz, CDCl₃) δ 8.31 (s, 1H), 7.35-7.27 (m, 4H), 7.20 (t, J=7.5 Hz, 3H), 6.88-6.61 (m, 1H), 5.76 (s, 1H), 4.01-3.95 (m, 4H), 3.55-3.49 (m, 6H), 2.70 (t, J=7.0 Hz, 2H), 1.79-1.68 (m, 4H). LC-MS m/z: 379.2 [M+H]⁺. HPLC Purity (214 nm): 94%; t_{R}=8.77 min.

### Example 138—4-Cyano-2-methoxy-N-(4-phenylbutyl)-1H-benzo[d]imidazole-1-carboxamide

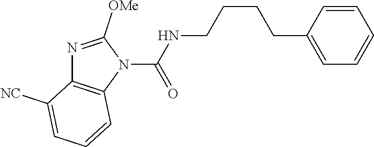 [SMILES: COc1nc2c(C#N)cccc2n1C(=O)NCCCCc1ccccc1]

Following general procedure C, 2-methoxy-1H-benzo[d]imidazole-4-carbonitrile (100 mg, 0.57 mmol) and 4-phenylbutan-1-amine (76.5 mg, 0.51 mmol) afforded the title compound (61.0 mg, 30.7%) as a white solid. ¹H NMR (400 MHz, CDCl₃) δ 8.38 (d, J=7.4 Hz, 1H), 7.54 (d, J=6.9 Hz, 1H), 7.32-7.15 (m, 6H), 6.96-6.88 (m, 1H), 4.39 (s, 3H), 3.47 (q, J=6.6 Hz, 2H), 2.69 (t, J=7.1 Hz, 2H), 1.74-1.71 (m, 4H). LC-MS m/z: 349.1 [M+H]⁺. HPLC Purity (214 nm): 99%; t_{R}=9.21 min.

### Example 139—4-Cyano-N-((4,4-difluorocyclohexyl)methyl)-2-methoxy-1H-benzo[d]imidazole-1-carboxamide

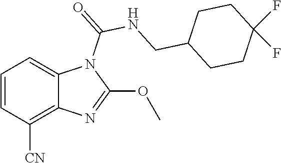 [SMILES: COc1nc2c(C#N)cccc2n1C(=O)NCC1CCC(F)(F)CC1]

Following general procedure F, 3-amino-2-nitrobenzonitrile (1.0 g, 6.1 mmol) afforded crude 2,3-diaminobenzonitrile (1.0 g, 98%). LC-MS m/z: 134 [M+H]⁺. Purity (214 nm): 81%; t_{R}=1.53 min.

Following general procedure G (method A), 2,3-diaminobenzonitrile (1.0 g, 7.5 mmol) afforded 2-methoxy-1H-benzo[d]imidazole-4-carbonitrile (800 mg, 61.6%) as a yellow solid. LC-MS m/z: 174 [M+H]⁺. Purity (254 nm): 95%; t_{R}=1.6 min.

Following general procedure C, 2-methoxy-1H-benzo[d]imidazole-4-carbonitrile (123 mg, 0.71 mmol) and (4,4-difluorocyclohexyl)methanamine (95.5 mg, 0.64 mmol) the title compound (78.1 mg, 31.7%) as a white solid. ¹H NMR (400 MHz, CDCl₃) δ 8.37 (d, J=8.3 Hz, 1H), 7.53 (d, J=7.6 Hz, 1H), 7.28 (t, J=7.6 Hz, 1H), 7.08 (s, 1H), 4.43 (s, 3H), 3.37 (t, J=6.4 Hz, 2H), 2.16-2.08 (m, 2H), 1.92-1.61 (m, 5H), 1.43-1.37 (m, 2H). LC-MS m/z: 349.1 [M+H]⁺. HPLC Purity (214 nm): 98.73%; t_{R}=9.26 min.

### Example 140—4-Cyano-2-methoxy-N-(4-methylpent-2-ynyl)-1H-benzo[d]imidazole-1-carboxamide

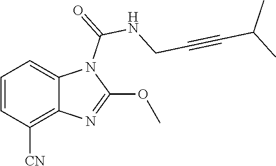 [SMILES: COc1nc2c(C#N)cccc2n1C(=O)NCC#CC(C)C]

Following general procedure C, 2-methoxy-1H-benzo[d]imidazole-4-carbonitrile (100 mg, 0.58 mmol) and 4-methylpent-2-yn-1-amine hydrochloride (67.33 mg, 0.69 mmol) afforded the title compound (75.8 mg, 46.2%) as a white solid. ¹H NMR (400 MHz, CDCl₃) δ 8.39 (d, J=8.2 Hz, 1H), 7.55 (d, J=7.8 Hz, 1H), 7.28 (t, J=8.1 Hz, 1H), 7.10 (s, 1H), 4.44 (s, 3H), 4.22 (dd, J=5.1, 1.9 Hz, 2H), 2.59 (dq, J=13.9, 6.9 Hz, 1H), 1.17 (d, J=7.2 Hz, 6H). LC-MS m/z: 297.0 [M+H]⁺. HPLC Purity (214 nm): 98%; t_{R}=6.31 min.

### Example 141—N-(3-Cyclopropylpropyl)-4-morpholino-1H-benzo[d]imidazole-1-carboxamide

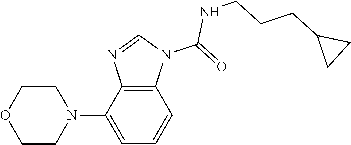 [SMILES: O=C(NCCCC1CC1)n1cnc2c(N3CCOCC3)cccc21]

Following general procedure C. 4-(1H-benzo[d]imidazol-4-yl)-morpholine (102 mg, 0.5 mmol) and 3-cyclopropylpropan-1-amine (66.0 mg, 0.5 mmol) afforded the title compound (29.6 mg, 18.3%) as a white solid. ¹H NMR (400 MHz, CDCl₃) δ 8.34 (s, 1H), 7.33-7.28 (m, 2H), 6.77-6.73 (m, 1H), 5.77 (bs, 1H), 3.99 (t, J=4.8 Hz, 4H), 3.60-3.53 (m, 6H), 1.84 (p, J=7.2 Hz, 2H), 1.37 (q, J=7.6 Hz, 2H), 0.79-0.73 (m, 1H), 0.50-0.47 (m, 2H), 0.10-0.01 (m, 2H). LC-MS m/z: 329.1 [M+H]⁺. HPLC Purity (214 nm): 100%, t_{R}=8.19 min.

### Example 142—4-Morpholino-N-(3-phenylpropyl)-1H-benzo[d]imidazole-1-carboxamide

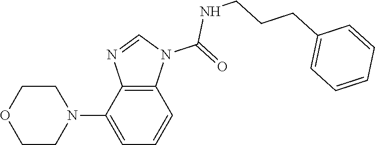 [SMILES: O=C(NCCCc1ccccc1)n1cnc2c(N3CCOCC3)cccc21]

Following general procedure C, 4-(1H-benzo[d]imidazol-4-yl)morpholine (102 mg, 0.5 mmol) and 3-phenylpropan-1-amine (67.5 mg, 0.5 mmol) afforded the title compound (33.3 mg, 18.3%) as a white solid. ¹H NMR (400 MHz, CDCl₃) δ 8.18 (s, 1H), 7.34-7.21 (m, 7H), 6.73 (dd, J=7.2, 1.5 Hz, 1H), 5.66 (s, 1H), 3.99-3.95 (m, 4H), 3.59-3.49 (m, 6H), 2.78 (t, J=7.4 Hz, 2H), 2.06 (p, J=7.5 Hz, 2H). LC-MS m/z: 365.3 [M+H]⁺. HPLC Purity (214 nm): 100%; t_{R}=8.37 min.

### Example 143—4-(4,4-Difluoropiperidin-1-yl)-N-isopentyl-1H-benzo[d]imidazole-1-carboxamide

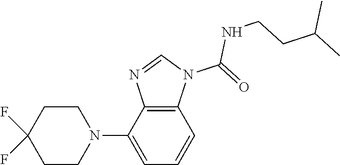 [SMILES: CC(C)CCNC(=O)n1cnc2c(N3CCC(F)(F)CC3)cccc21]

Following general procedure E, 3-fluoro-2-nitroaniline (3.0 g, 19.8 mmol) and 4,4-difluoropiperidine (6.23 g, 39.7 mmol) afforded 3-(4,4-difluoropiperidin-1-yl)-2-nitroaniline (2.5 g, 50.0%) as a brown oil. LC-MS m/z: 258.1 [M+H]⁺. Purity (214 nm): 90%; t_{R}=1.48 min.

Following general procedure F, 3-(4,4-difluoropiperidin-1-yl)-2-nitroaniline (2.5 g, 9.72 mmol) afforded crude 3-(4,4-difluoropiperidin-1-yl)benzene-1,2-diamine (1.8 g, 81.1%) as a brown solid. LC-MS m/z: 228.1 [M+H]⁺. Purity (214 nm): 90%; t_{R}=1.76 min.

Following general procedure G (method B), 3-(4,4-difluoropiperidin-1-yl)benzene-1,2-diamine (400 mg, 1.76 mmol) afforded 4-(4,4-difluoropiperidin-1-yl)-1H-benzo[d]imidazole (400 mg, 96%) as a yellow solid. LC-MS m/z: 238.0 [M+H]⁺. Purity (254 nm): 67%; t_{R}=1.70 min.

Following general procedure C, 4-(4,4-difluoropiperidin-1-yl)-1H-benzo[d]imidazole (200 mg, 0.84 mmol) and 3-methylbutan-1-amine (110 mg, 1.27 mmol) afforded the title compound (84.5 mg, 29%) as a white solid. ¹H NMR (400 MHz, CDCl₃) δ 8.34 (s, 1H), 7.31-7.28 (m, 2H), 6.77 (dd, J=7.0, 1.7 Hz, 1H), 5.71 (s, 1H), 3.67 (t, J=5.6 Hz, 4H), 3.54 (dd, J=14.7, 5.9 Hz, 2H), 2.27-2.16 (m, 4H), 1.72 (dq, J=13.4, 6.8 Hz, 1H), 1.62 (q, J=7.3 Hz, 2H), 0.99 (d, J=6.6 Hz, 6H). LC-MS m/z: 351.2 [M+H]⁺. HPLC Purity (214 nm): 98%; t_{R}=9.79 min.

### Example 144—4-(4,4-Difluoropiperidin-1-yl)-N-isopentyl-2-methoxy-1H-benzo[d]imidazole-1-carboxamide

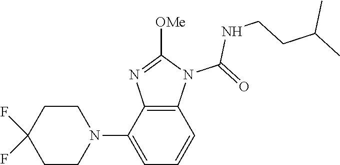 [SMILES: COc1nc2c(N3CCC(F)(F)CC3)cccc2n1C(=O)NCCC(C)C]

Following general procedure C, 4-(4,4-difluoropiperidin-1-yl)-2-methoxy-1H-benzo[d]imidazole (300 mg, 1.12 mmol) and 3-methylbutan-1-amine (131.8 mg, 1.69 mmol) afforded the title compound (54.7 mg, 12.7%) as a yellow solid. ¹H NMR (400 MHz, CDCl₃) δ 7.83 (d, J=8.4 Hz, 1H), 7.14 (t, J=8.0 Hz, 1H), 6.94 (s, 1H), 6.74 (d, J=8.1 Hz, 1H), 4.33 (s, 3H), 3.54 (t, J=5.6 Hz, 4H), 3.48-3.41 (m, 2H), 2.26-2.21 (m, 4H), 1.75-1.61 (m, 1H), 1.50-1.41 (m, 2H), 0.97 (d, J=6.6 Hz, 6H). LC-MS m/z: 381.0 [M+H]⁺. HPLC Purity (214 nm): 100%; t_{R}=10.38 min.

### Example 145—N-iso-Pentyl-2-methoxy-4-(4-methylpiperazin-1-yl)-1H-benzo[d]imidazole-1-carboxamide

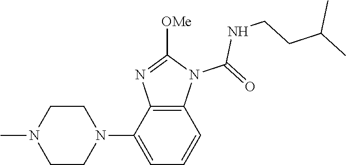 [SMILES: COc1nc2c(N3CCN(C)CC3)cccc2n1C(=O)NCCC(C)C]

Following general procedure E, 3-fluoro-2-nitroaniline (1.0 g, 6.41 mmol) and 1-methylpiperazine (1.6 g, 16.03 mmol) afforded 3-(4-methylpiperazin-1-yl)-2-nitroaniline (1.3 g, 87%) as an orange solid. LC-MS m/z: 237.1 [M+H]⁺. Purity (254 nm): 99%; t_{R}=1.60 min.

Following general procedure E, 3-(4-methylpiperazin-1-yl)-2-nitroaniline (1.3 g, 5.51 mmol) afforded crude 3-(4-methylpiperazin-1-yl)benzene-1,2-diamine (1.0 g, 88%) as a brown solid. LC-MS m/z: 207.2 [M+H]⁺. Purity (254 nm): 96%; t_{R}=1.29 min.

Following general procedure G (method A), 3-(4-methylpiperazin-1-yl)benzene-1,2-diamine (750 mg, 3.64 mmol) afforded 2-methoxy-4-(4-methylpiperazin-1-yl)-1H-benzo[d]imidazole (210 mg, 23%) as a white solid. LC-MS m/z: 247.1 [M+H]⁺. Purity (214 nm): 99%; t_{R}=1.30 min.

Following general procedure C, 2-methoxy-4-(4-methylpiperazin-1-yl)-1H-benzo[d]imidazole (200 mg, 0.81 mmol) and 3-methylbutan-1-amine (106 mg, 1.22 mmol) afforded the title compound (16.8 mg, 6%) as a yellow oil. ¹H NMR (400 MHz, CDCl₃) δ 7.81 (d, J=8.3 Hz, 1H), 7.13 (t, J=8.2 Hz, 1H), 6.93 (s, 1H), 6.73 (d, J=7.9 Hz, 1H), 4.31 (s, 3H), 3.47-3.40 (m, 6H), 2.68 (t, J=4.6 Hz, 4H), 2.38 (s, 3H), 1.69 (dq, J=13.4, 6.7 Hz, 1H), 1.54 (q, J=7.6 Hz, 2H), 0.97 (d, J=6.6 Hz, 6H). LC-MS m/z: 360.3 [M+H]⁺. Purity (214 nm): 99%; t_{R}=5.90 min.

### Example 146—N-iso-Pentyl-2-methoxy-4-morpholino-1H-benzo[d]imidazole-1-carboxamide

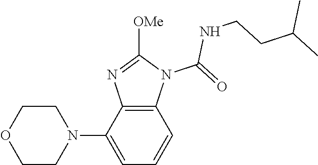 [SMILES: COc1nc2c(N3CCOCC3)cccc2n1C(=O)NCCC(C)C]

Following general procedure C, 4-(2-methoxy-1H-benzo[d]imidazol-4-yl)morpholine (300 mg, 1.28 mmol) and 3-methylbutan-1-amine (168 mg, 1.93 mmol) afforded the title compound 145 mg, 34.6%) as a yellow solid. ¹H NMR (400 MHz, CDCl₃) δ 7.84 (d, J=8.2 Hz, 1H), 7.15 (t, J=8.1 Hz, 1H), 6.95 (s, 1H), 6.72 (d, J=8.1 Hz, 1H), 4.31 (s, 3H), 3.97 (t, J=4.4 Hz, 4H), 3.51-3.39 (m, 6H), 1.69 (dq, J=13.4, 6.7 Hz, 1H), 1.58-1.52 (m, 2H), 0.97 (d, J=6.6 Hz, 6H). LC-MS m/z: 347.10 [M+H]⁺. HPLC Purity (214 nm): 95.29%; t_{R}=9.24 min.

### Example 147—4-(4-Cyclopropylpiperazin-1-yl)-N-iso-pentyl-1H-benzo[d]imidazole-1-carboxamide

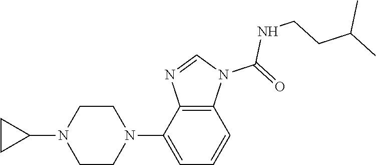 [SMILES: CC(C)CCNC(=O)n1cnc2c(N3CCN(C4CC4)CC3)cccc21]

Following general procedure E, 3-fluoro-2-nitroaniline (1.5 g, 9.6 mmol) and 1-cyclopropyl piperazine (484 g, 3.84 mmol) afforded 3-(4-cyclopropylpiperazin-1-yl)-2-nitroaniline as a white solid (1.2 g). LC-MS m/z: 263.7 [M+H]⁺.

Following general procedure F, 3-(4-cyclopropylpiperazin-1-yl)-2-nitroaniline (1.2 g, 4.6 mmol) afforded crude 3-(4-cyclopropylpiperazin-1-yl)benzene-1,2-diamine as a white solid (1.1 g). LC-MS m/z: 233.7 [M+H]⁺.

Following general procedure G (method B), 3-(4-cyclopropylpiperazin-1-yl)benzene-1,2-diamine (1.1 g, 4.6 mmol) afforded crude 4-(4-cyclopropylpiperazin-1-yl)-1H-benzo[d]imidazole (700 mg) as a white solid. LC-MS m/z: 243.7 [M+H]⁺. Purity (214 nm)

Following general procedure C, 4-(4-cyclopropylpiperazin-1-yl)-1H-benzo[d]imidazole (200 mg, 0.8 mmol) and 3-methylbutan-1-amine (84 mg, 0.96 mmol) afforded the title compound (65.4 mg, 22.3%) as a white solid. ¹H NMR (400 MHz, CDCl₃) δ 8.39 (s, 1H), 7.31 (dd, J=12.0, 7.7 Hz, 2H), 6.74 (d, J=7.4 Hz, 1H), 5.90 (s, 1H), 3.61-3.52 (m, 6H), 3.13-2.98 (m, 4H), 1.92-1.82 (m, 1H), 1.72 (dq, J=13.4, 6.8 Hz, 1H), 1.59 (dd, J=14.8, 7.0 Hz, 2H), 0.99 (d, J=6.6 Hz, 6H), 0.68-0.55 (m, 4H). LC-MS m/z: 356.1 [M+H]⁺. HPLC Purity (214 nm): 96.35%; t_{R}=6.60 min.

### Example 148—N-Cyclohexyl-4-(4-cyclopropylpiperazin-1-yl)-1H-benzo[d]imidazole-1-carboxamide

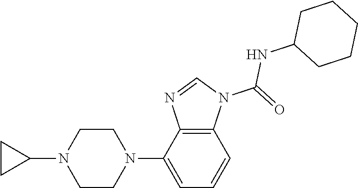 [SMILES: O=C(NC1CCCCC1)n1cnc2c(N3CCN(C4CC4)CC3)cccc21]

Following general procedure C, 4-(4-cyclopropylpiperazin-1-yl)-1H-benzo[d]imidazole (200 mg, 0.8 mmol) and cyclohexanamine (95 mg, 0.96 mmol) afforded the title compound (87.1 mg, 28.1%) as a white solid. ¹H NMR (400 MHz, CDCl₃) δ 8.36 (s, 1H), 7.31-7.27 (m, 2H), 6.75 (d, J=6.9 Hz, 1H), 5.66 (d, J=6.9 Hz, 1H), 3.92 (s, 1H), 3.54 (bs, 5H), 2.99 (t, J=4.8 Hz, 4H), 2.12 (d, J=10.0 Hz, 2H), 1.80 (bs 2H), 1.56-1.23 (m, 6H), 0.65-0.54 (m, 4H). LC-MS m/z: 368.3 [M+H]⁺. Purity (214 nm): 100%; t_{R}=6.61 min.

### Example 149—N-Cyclohexyl-4-morpholino-1H-benzo[d]imidazole-1-carboxamide

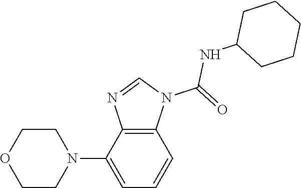 [SMILES: O=C(NC1CCCCC1)n1cnc2c(N3CCOCC3)cccc21]

Following general procedure C, 4-(1H-benzo[d]imidazol-4-yl)-morpholine (100 mg, 0.49 mmol) and cyclohexanamine (48.8 mg, 0.49 mmol) afforded the title compound (24.2 mg, 15.0%) as a white solid. ¹H NMR (400 MHz, CDCl₃) δ 8.36 (s, 1H), 7.32 (d, J=6.8 Hz, 2H), 6.76 (dd, J=6.5, 2.2 Hz, 1H), 5.64 (d, J=7.8 Hz, 1H), 4.02-3.95 (m, 5H), 3.68-3.52 (m, 4H), 2.14 (bs, 2H), 1.85-1.78 (m, 2H), 1.70 (bs, 2H), 1.51-1.23 (m, 4H). LC-MS m/z: 329.0 [M+H]⁺. HPLC Purity (214 nm): 1000%; t_{R}=7.88 min.

### Example 150—4-(4-Cyclopropylpiperazin-1-yl)-N-(3-phenylpropyl)-1H-benzo[d]imidazole-1-carboxamide

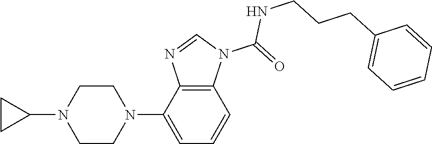 [SMILES: O=C(NCCCc1ccccc1)n1cnc2c(N3CCN(C4CC4)CC3)cccc21]

Following general procedure C, 4-(4-cyclopropylpiperazin-1-yl)-1H-benzo[d]imidazole (200 mg, 0.8 mmol) and 3-phenylpropan-1-amine (130 mg, 0.96 mmol) afforded the title compound (49.5 mg, 14.9%) as a white solid. ¹H NMR (400 MHz, CDCl₃) δ 8.22 (s, 1H), 7.34-7.28 (m, 2H), 7.26-7.20 (m, 5H), 6.73 (d, J=7.7 Hz, 1H), 5.69 (s, 1H), 3.61-3.50 (m, 6H), 2.98-2.92 (m, 4H), 2.78 (t, J=7.4 Hz, 2H), 2.06 (p, J=7.2 Hz, 2H), 1.79 (bs, 1H), 0.60-0.53 (m, 4H). LC-MS m/z: 404.1 [M+H]⁺. Purity (214 nm): 100%; t_{R}=6.85 min.

### Example 151—N-iso-Pentyl-4-(piperidin-1-yl)-1H-benzo[d]imidazole-1-carboxamide

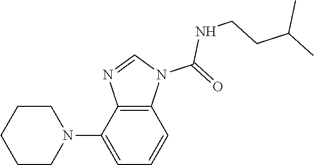 [SMILES: CC(C)CCNC(=O)n1cnc2c(N3CCCCC3)cccc21]

Following general procedure E, 3-fluoro-2-nitroaniline (650 mg, 4.16 mmol) and piperidine (532 mg, 6.24 mmol) afforded 2-nitro-3-(piperidin-1-yl)aniline (660 mg, 66%) as a brown oil. LC-MS m/z: 222.1 [M+H]⁺. Purity (214 nm): 100%; t_{R}=1.31 min.

Following general procedure F, 2-nitro-3-(piperidin-1-yl)aniline (660 mg, 2.98 mmol) afforded crude 3-(piperidin-1-yl)benzene-1,2-diamine (580 mg) as a brown oil. LC-MS m/z: 192.3 [M+H]⁺. Purity (254 nm): 95%; t_{R}=1.02 min.

Following general procedure G (method B), 3-(piperidin-1-yl)benzene-1,2-diamine (580 mg, 3.03 mmol) afforded crude 4-(piperidin-1-yl)-1H-benzo[d]imidazole (520 mg) as a grey solid. LC-MS m/z: 202.1 [M+H]⁺. Purity (214 nm): 95%; t_{R}=1.59 min.

Following general procedure C, 4-(piperidin-1-yl)-1H-benzo[d]imidazole (200 mg, 0.99 mmol) and 3-methylbutan-1-amine (87 mg, 0.99 mmol) afforded the title compound (31.1 mg, 10%) as a white solid. ¹H NMR (400 MHz, CDCl₃) δ 8.34 (s, 1H), 7.31-7.22 (m, 2H), 6.75 (d, J=7.7 Hz, 1H), 5.72 (s, 1H), 3.53 (dt, J=7.5, 5.8 Hz, 2H), 3.49 (t, J=5.6 Hz, 4H), 1.89-1.82 (m, 4H), 1.78-1.73 (m, 1H), 1.69-1.61 (m, 2H), 1.60-1.56 (m, 2H), 0.99 (d, J=6.6 Hz, 6H). LC-MS m/z: 315.1 [M+H]⁺. HPLC Purity (214 nm): 100%; t_{R}=6.86 min.

### Example 152—4-(4-(Cyclopropylmethyl)piperazin-1-yl)-N-iso-pentyl-1H-benzo[d]imidazole-1-carboxamide

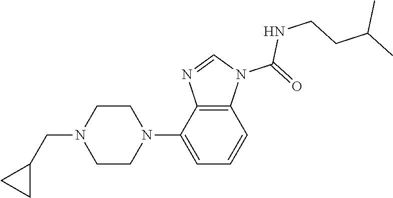 [SMILES: CC(C)CCNC(=O)n1cnc2c(N3CCN(CC4CC4)CC3)cccc21]

Following general procedure E, 3-fluoro-2-nitroaniline (450 mg, 2.88 mmol) and (cyclopropylmethyl)piperazine (606 mg, 4.32 mmol) afforded 3-(4-(cyclopropylmethyl) piperazin-1-yl)-2-nitroaniline (850 mg, 96%) as a brown oil. LC-MS m/z: 277.0 [M+H]⁺. Purity (214 nm): 100%; t_{R}=1.40 min.

Following general procedure F, 3-(4-(cyclopropylmethyl)piperazin-1-yl)-2-nitroaniline (812 mg, 2.938 mmol) afforded crude 3-(4-(cyclopropylmethyl)piperazin-1-yl)benzene-1,2-diamine (620 mg) as a brown oil. LC-MS m/z: 247.2 [M+H]⁺. Purity (214 nm): 94%; t_{R}=1.45 min.

Following general procedure G (method B), 3-(4-(cyclopropylmethyl)piperazin-1-yl)benzene-1,2-diamine (620 mg, 2.52 mmol) afforded crude 4-(4-(cyclopropylmethyl)piperazin-1-yl)-1H-benzo[d]imidazole (620 mg) as a grey solid. LC-MS m/z: 257.1 [M+H]⁺. Purity (214 nm): 97.7%; t_{R}=1.43 min.

Following general procedure C, 4-(4-(cyclopropylmethyl)piperazin-1-yl)-1H-benzo[d]imidazole (200 mg, 0.78 mmol) and 3-methylbutan-1-amine (68 mg, 0.78 mmol) afforded the title compound (72.5 mg, 25.1%) as a yellow solid. ¹H NMR (400 MHz, CDCl₃) δ 8.35 (s, 1H), 7.31-7.27 (m, 2H), 6.80-6.75 (m, 1H), 5.80 (s, 1H), 3.58 (bs, 4H), 3.53 (dd, J=14.8, 6.0 Hz, 2H), 2.85 (t, J=4.4 Hz, 4H), 2.37 (d, J=6.6 Hz, 2H), 1.71 (septep J=7.2 Hz, 1H), 1.59 (q, J=7.1 Hz, 2H), 0.99 (d, J=6.6 Hz, 6H), 0.98-0.91 (m, 1H), 0.59-0.54 (m, 2H), 0.16 (q, J=5.6 Hz, 2H). LC-MS m/z: 370.2 [M+H]⁺. HPLC Purity (214 nm): 96.49%; t_{R}=6.82 min.

### Example 153—N-iso-Pentyl-4-(4-(2,2,2-trifluoroethyl)piperazin-1-yl)-1H-benzo[d]imidazole-1-carboxamide

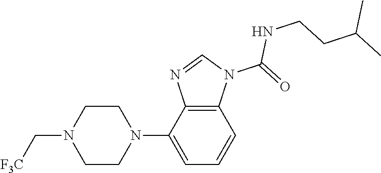 [SMILES: CC(C)CCNC(=O)n1cnc2c(N3CCN(CC(F)(F)F)CC3)cccc21]

Following general procedure E, 3-fluoro-2-nitroaniline (450 mg, 2.88 mmol) and 1-(2,2,2-trifluoroethyl)piperazine hydrochloride (834 mg, 3.46 mmol) afforded 2-nitro-3-(4-(2,2,2-trifluoroethyl)piperazin-1-yl)aniline (880 mg, 90%) as a brown oil. LC-MS m/z: 305.0 [M+H]⁺. Purity (214 nm): 93.47%; t_{R}=1.97 min.

Following general procedure F, 2-nitro-3-(4-(2,2,2-trifluoroethyl)piperazin-1-yl)aniline (880 mg, 2.892 mmol) afforded crude 3-(4-(2,2,2-trifluoroethyl)piperazin-1-yl)benzene-1,2-diamine (840 mg) as a brown semisolid. LC-MS m/z: 275.2 [M+H]⁺. Purity (254 nm): 91.2%; t_{R}=1.75 min.

Following general procedure G (method B), 3-(4-(2,2,2-trifluoroethyl)piperazin-1-yl)benzene-1,2-diamine (840 mg, 3.06 mmol) afforded crude 4-(4-(2,2,2-trifluoroethyl)piperazin-1-yl)-1H-benzo[d]imidazole (810 mg) as a brown solid. LC-MS m/z: 285.1 [M+H]⁺. Purity (214 nm): 97.95%; t_{R}=1.70 min.

Following general procedure C, 4-(4-(2,2,2-trifluoroethyl)piperazin-1-yl)-1H-benzo[d]imidazole (200 mg, 0.703 mmol) and 3-methylbutan-1-amine (61 mg, 0.703 mmol) afforded the title compound (37.4 mg, 13.4%) as a white solid. ¹H NMR (400 MHz, CDCl₃) δ 8.36 (s, 1H), 7.29 (dd, J=5.9, 4.8 Hz, 2H), 6.78-6.72 (m, 1H), 5.75 (s, 1H), 3.62-3.53 (m, 6H), 3.07 (q, J=9.6 Hz, 2H), 3.01-2.94 (m, 4H), 1.79-1.68 (m, 1H), 1.61-1.54 (m, 2H), 0.99 (d, J=6.6 Hz, 6H). LC-MS m/z: 398.1 [M+H]⁺. HPLC Purity (214 nm): 96.27%; t_{R}=8.29 min.

### Example 154—N-iso-Pentyl-4-(4-(oxetan-3-yl)piperazin-1-yl)-1H-benzo[d]imidazole-1-carboxamide

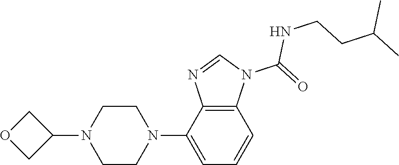 [SMILES: CC(C)CCNC(=O)n1cnc2c(N3CCN(C4COC4)CC3)cccc21]

To a solution 4-(piperazin-1-yl)-1H-benzo[d]imidazole (500 mg, 2.48 mmol) in MeOH (10 mL) was added oxetan-3-one (215 mg, 2.98 mmol) and the mixture was stirred at RT for 1 h. Then NaBH₃CN (460 mg, 7.44 mmol) was added at 0° C. and the solution was stirred at RT for 16 h. Water (10 ml) was added and the mixture was extracted with EA (50 mL). The organic layers were concentrated and purified by silica gel column chromatography (DCM/MeOH=20/1) to give 4-(4-(oxetan-3-yl)piperazin-1-yl)-1H-benzo[d]imidazole (460 mg, 72%) as a red oil. LC-MS m/z: 259.2 [M+H]⁺. HPLC Purity (254 nm): 98.54%; t_{R}=1.42 min.

Following general procedure C, 4-(4-(oxetan-3-yl)piperazin-1-yl)-1H-benzo[d]imidazole (200 mg, 0.78 mmol) and 3-methylbutan-1-amine (68 mg, 0.78 mmol) afforded the title compound (54.3 mg, 18.9%) as a black oil. ¹H NMR (400 MHz, CDCl₃) δ 8.37 (s, 1H), 7.35-7.30 (m, 2H), 6.78 (dd, J=5.7, 3.1 Hz, 1H), 5.80 (s, 1H), 4.85-4.64 (m, 4H), 4.78-4.62 (m, 4H), 3.68-3.57 (m, 5H), 3.55 (q, J=8.8 Hz, 2H), 2.69-2.62 (m, 4H), 1.74 (sep, J=6.7 Hz, 1H), 1.61 (q, J=7.6 Hz, 2H), 1.01 (d, J=6.6 Hz, 6H). LC-MS m/z: 372.1 [M+H]⁺. HPLC Purity (214 nm): 100.0%; t_{R}=6.42 min.

### Example 155—N-iso-Pentyl-4-(4-(tetrahydro-2H-pyran-4-yl)-piperazin-1-yl)-1H-benzo[d]imidazole-1-carboxamide

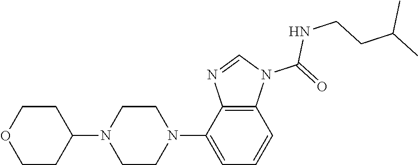 [SMILES: CC(C)CCNC(=O)n1cnc2c(N3CCN(C4CCOCC4)CC3)cccc21]

Following general procedure E, 3-fluoro-2-nitroaniline (720 mg, 4.61 mmol) and 1-(tetrahydro-2H-pyran-4-yl)piperazine (922 mg, 3.84 mmol) afforded 2-nitro-3-(4-(tetrahydro-2H-pyran-4-yl)piperazin-1-yl)aniline (886 mg, 78.4%) as a red solid. LC-MS m/z: 307.1 [M+H]⁺. Purity (214 nm): 93.97%; t_{R}=1.20 min.

Following general procedure F, 2-nitro-3-(4-(tetrahydro-2H-pyran-4-yl)piperazin-1-yl)aniline (886 mg, 2.9 mmol) afforded 3-(4-(tetrahydro-2H-pyran-4-yl)piperazin-1-yl)benzene-1,2-diamine (402 mg, 50.31%) as a solid. LC-MS m/z: 277.2 [M+H]⁺. Purity (214 nm): 95.9%; t_{R}=1.45 min.

Following general procedure G (method B), 3-(4-(tetrahydro-2H-pyran-4-yl)piperazin-1-yl)benzene-1,2-diamine (402 mg, 1.46 mmol) afforded 4-(4-(tetrahydro-2H-pyran-4-yl)piperazin-1-yl)-1H-benzo[d]imidazole (320 mg, 77%) as a brown solid. LC-MS m/z: 287.2 [M+H]⁺. HPLC Purity (214 nm): 76.54%; t_{R}=1.44 min.

Following general procedure C, 4-(4-(tetrahydro-2H-pyran-4-yl)piperazin-1-yl)-1H-benzo[d]imidazole (132 mg, 0.46 mmol) and iso-butylamine (45 mg, 0.46 mmol) afforded the title compound (46.2 mg, 22.1%) as a white solid. ¹H NMR (400 MHz, CDCl₃) δ 8.56 (s, 1H), 7.54 (s, 1H), 7.29 (d, J=4.7 Hz, 1H), 6.94 (s, 1H), 6.74 (d, J=7.7 Hz, 1H), 4.10 (d, J=8.9 Hz, 2H), 3.71 (bs, 4H), 3.57-3.40 (m, 4H), 3.25 (bs, 4H), 3.07 (s, 1H), 1.98 (d, J=11.2 Hz, 2H), 1.93-1.79 (m, 2H), 1.79-1.69 (m, 1H), 1.58 (q, J=7.1 Hz, 2H), 0.97 (d, J=6.5 Hz, 6H). LC-MS m/z: 400.2 [M+H]⁺. HPLC Purity (214 nm): 100%; t_{R}=6.55 min.

### Example 156—4-(4-(Imidazo[1,5-a]pyridin-5-yl)piperazin-1-yl)-N-iso-pentyl-1H-benzo[d]imidazole-1-carboxamide

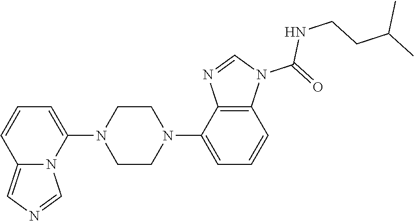 [SMILES: CC(C)CCNC(=O)n1cnc2c(N3CCN(c4cccc5cncn45)CC3)cccc21]

To a solution of 5-bromoimidazo[1,5-a]pyridine (400 mg, 2.04 mmol) and piperazine (900 mg, 10.2 mmol) in NMP (4 mL) was added EtN(i-Pr)₂ (800 mg, 6.12 mmol) and the mixture was stirred at 200° C. for 4 h under microwave condition. The mixture was concentrated and purified by silica gel column chromatography (PE/EA=20/1) to give 5-(piperazin-1-yl)imidazo[1,5-a]pyridine (300 mg, 71.4%) as a yellow oil. LC-MS m/z: 203.1 [M+H]⁺. Purity (214 nm): >99%: t_{R}=1.37 min.

Following general procedure E, 3-fluoro-2-nitroaniline (234 mg, 1.5 mmol) and 5-(piperazin-1-yl)imidazo[1,5-a]pyridine (300 mg, 1.5 mmol) afforded 3-(4-(imidazo[1,5-a]pyridin-5-yl)piperazin-1-yl)-2-nitroaniline (300 mg, 59.8%) as a yellow oil. LC-MS m/z: 339.1 [M+H]⁺. Purity (214 nm): 82.89%; t_{R}=1.65 min.

Following general procedure F, 3-(4-(imidazo[1,5-a]pyridin-5-yl)piperazin-1-yl)-2-nitroaniline (300 mg, 0.88 mmol) afforded crude 3-(4-(imidazo[1,5-a]pyridin-5-yl)piperazin-1-yl)benzene-1,2-diamine (100 mg, 35.4%) as a yellow oil. LC-MS m/z: 309.1 [M+H]⁺. HPLC Purity (214 nm): 55%; t_{R}=1.57 min.

Following general procedure G (method B), 3-(4-(imidazo[1,5-a]pyridin-5-yl)piperazin-1-yl)benzene-1,2-diamine (100 mg, 0.32 mmol) afforded 4-(4-(imidazo[1,5-a]pyridin-5-yl)piperazin-1-yl)-1H-benzo[d]imidazole (100 mg, 97%) as a yellow oil. LC-MS m/z: 319.1 [M+H]⁺. Purity (214 nm): 80%; t_{R}=1.50 min.

Following general procedure C, 4-(4-(imidazo[1,5-a]pyridin-5-yl)-piperazin-1-yl)-1H-benzo[d]imidazole (90 mg, 0.28 mmol) and 3-methylbutan-1-amine (25 mg, 0.28 mmol) afforded the title compound (2.8 mg, 2.3%) as a white solid. ¹H NMR (400 MHz, CDCl₃) δ 8.38 (s, 1H), 8.22 (s, 1H), 7.48 (s, 1H), 7.35 (d, J=6.8 Hz, 2H), 7.25 (s, 1H), 6.85-6.76 (m, 2H), 6.16 (d, J=6.7 Hz, 1H), 5.83 (s, 1H), 3.77 (bs, 4H), 3.58-3.53 (m, 2H), 3.41 (t, J=4.6 Hz, 4H), 1.73-1.64 (m, 1H), 1.64 (bs, 2H), 1.00 (d, J=6.6 Hz, 6H). LC-MS m/z: 432.2 [M+H]⁺. HPLC Purity (214 nm): 97.36%; t_{R}=8.42 min.

### Example 157—N-iso-Pentyl-4-(4-methyl-4,7-diazaspiro[2.5]-octan-7-yl)-1H-benzo[d]imidazole-1-carboxamide

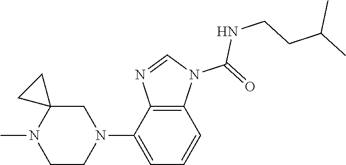 [SMILES: CC(C)CCNC(=O)n1cnc2c(N3CCN(C)C4(CC4)C3)cccc21]

Following general procedure E, 3-fluoro-2-nitroaniline (295 mg, 1.89 mmol) and tert-butyl 4,7-diazaspiro[2.5]octane-4-carboxylate (400 mg, 1.89 mmol) afforded tert-butyl 7-(3-amino-2-nitrophenyl)-4,7-diazaspiro[2.5]octane-4-carboxylate (500 mg, 76.1%) as a red oil. LC-MS m/z: 349.2 [M+H]⁺. Purity (254 nm): 100.0%; t_{R}=2.03 min.

Following general procedure F, tert-butyl 7-(3-amino-2-nitrophenyl)-4,7-diazaspiro[2.5]octane-4-carboxylate (500 mg, 1.44 mmol) afforded tert-butyl 7-(2,3-diaminophenyl)-4,7-diazaspiro[2.5]octane-4-carboxylate (400 mg, 87.5%) as a red oil. LC-MS m/z: 319.2 [M+H]⁺. HPLC Purity (214 nm): 96.65%; t_{R}=1.77 min.

Following general procedure G (method B), tert-butyl 7-(2,3-diaminophenyl)-4,7-diazaspiro[2.5]octane-4-carboxylate (400 mg, 1.26 mmol) afforded 4-(4,7-diazaspiro[2.5]octan-7-yl)-1H-benzo[d]-imidazole (280 mg, 98%) as a red oil. LC-MS m/z: 229.2 [M+H]⁺. Purity (254 nm): 91.75%; t_{R}=1.42 min.

To a solution of 4-(4,7-diazaspiro[2.5]octan-7-yl)-1H-benzo[d]imidazole (280 mg, 1.23 mmol) in MeOH (10 mL) was added HCHO (44 mg, 1.48 mmol) and the mixture was stirred at RT for 1 h. Then NaBH₃CN (232 mg, 3.69 mmol) was added at 0° C. and the solution was stirred at RT for 16 h. The reaction mixture was worked up, concentrated and purified by silica gel column chromatography (DCM/MeOH=20/1) to give 4-(4-methyl-4,7-diazaspiro[2.5]octan-7-yl)-1H-benzo[d]imidazole (180 mg, 60.6%) as a red oil. LC-MS m/z: 243.2 [M+H]⁺. Purity (254 nm): 100%; t_{R}=1.34 min.

Following general procedure C, 4-(4-methyl-4,7-diazaspiro[2.5]octan-7-yl)-1H-benzo[d]imidazole (180 mg, 0.74 mmol) and 3-methylbutan-1-amine (65 mg, 0.74 mmol) afforded the title compound (45.6 mg, 17.3%) as a white solid. ¹H NMR (400 MHz, CDCl₃) δ 8.35 (s, 1H), 7.32 (t, J=8.2 Hz, 1H), 7.30-7.28 (m, 1H), 6.72 (d, J=7.7 Hz, 1H), 6.01 (bs, 1H), 3.65 (bs, 2H), 3.58-3.54 (m, 4H), 3.30 (bs, 2H), 2.58 (s, 3H), 1.74 (septet, J=6.6 Hz, 1H), 1.61 (q, J=7.0 Hz, 2H), 1.05 (s, 2H), 1.01 (q, J=6.5 Hz, 6H), 0.80 (s, 2H). LC-MS m/z: 356.3 [M+H]⁺. HPLC Purity (214 nm): 100%; t_{R}=5.19 min.

### Example 158—4-(Hexahydropyrrolo[1,2-a]pyrazin-2(1H)-yl)-N-iso-pentyl-1H-benzo[d]imidazole-1-carboxamide

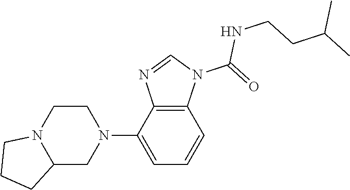 [SMILES: CC(C)CCNC(=O)n1cnc2c(N3CCN4CCCC4C3)cccc21]

Following general procedure E, 3-fluoro-2-nitroaniline (468 mg, 3.0 mmol) and octahydropyrrolo[1,2-a]pyrazine (378 mg, 3 mmol) afforded 3-(hexahydropyrrolo[1,2-a]-pyrazin-2(1H)-yl)-2-nitroaniline (350 mg) as a yellow solid. LC-MS m/z: 263.1 [M+H]⁺. Purity (214 nm): 83.4%; t_{R}=1.62 min.

Following general procedure F, 3-(hexahydropyrrolo[1,2-a]pyrazin-2(1H)-yl)-2-nitroaniline (262 mg, 1.0 mmol) afforded crude 3-(hexahydropyrrolo[1,2-a]pyrazin-2(1H)-yl)benzene-1,2-diamine (232 mg) as a yellow oil. LC-MS m/z: 233.2 [M+H]⁺. Purity (214 nm): 94.4%; t_{R}=1.38 min.

Following general procedure G (method B), 3-(hexahydropyrrolo[1,2-a]pyrazin-2(1H)-yl)benzene-1,2-diamine (232 mg, 1.0 mmol) afforded crude 4-(hexahydropyrrolo[1,2-a]pyrazin-2(1H)-yl)-1H-benzo[d]imidazole (200 mg) as a yellow oil. LC-MS m/z: 243.2 [M+H]⁺. Purity (214 nm): 79.4%; t_{R}=1.41 min.

Following general procedure C, 4-(hexahydropyrrolo[1,2-a]pyrazin-2(1H)-yl)-1H-benzo[d]imidazole (121 mg, 0.5 mmol) and 3-methylbutan-1-amine (51.0 mg, 0.5 mmol) afforded the title compound (86.9 mg, 48.90%) as a white solid. ¹H NMR (400 MHz, CDCl₃) δ 8.54 (d, J=5.0 Hz, 2H), 7.55 (d, J=8.2 Hz, 1H), 7.36 (s, 1H), 7.25 (t, J=8.4 Hz, 1H), 6.68 (d, J=7.6 Hz, 1H), 4.05 (bs, 2H), 3.60-3.28 (m, 5H), 3.25-2.98 (m, 3H), 2.75 (bs, 1H), 2.25-1.84 (m, 4H), 1.80-1.63 (m, 1H), 1.55 (q, J=7.2 Hz, 2H), 0.95 (t, J=6.4 Hz, 6H). LC-MS m/z: 356.2 [M+H]⁺. HPLC Purity (214 nm): 96.77%; t_{R}=6.66 min.

### Example 159—N-iso-Pentyl-4-(5-methyl-2,5-diazabicyclo[2.2.1]heptan-2-yl)-1H-benzo[d]imidazole-1-carboxamide

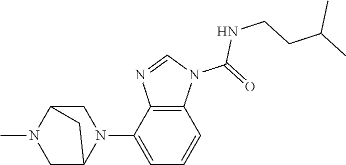 [SMILES: CC(C)CCNC(=O)n1cnc2c(N3CC4CC3CN4C)cccc21]

Following general procedure E, 2-methyl-2,5-diazabicyclo[2.2.1]heptane (600 mg, 5.36 mmol) and 3-fluoro-2-nitroaniline (836 mg, 5.36 mmol) afforded 3-(5-methyl-2,5-diazabicyclo[2.2.1]heptan-2-yl)-2-nitroaniline (700 mg, 52.6%) as a red oil. LC-MS m/z: 249.2 [M+H]⁺. Purity (214 nm): >99%; t_{R}=1.39 min.

Following general procedure F, 3-(5-methyl-2,5-diazabicyclo[2.2.1]heptan-2-yl)-2-nitroaniline (700 mg, 2.82 mmol) afforded 3-(5-methyl-2,5-diazabicyclo[2.2.1]heptan-2-yl)benzene-1,2-diamine (650 mg, >99%) as a white solid. LC-MS m/z: 219.3 [M+H]⁺. Purity (214 nm): 89.33%; t_{R}=0.66 min.

Following general procedure G (method B), 3-(5-methyl-2,5-diazabicyclo[2.2.1]heptan-2-yl)benzene-1,2-diamine (650 mg, 2.98 mmol) afforded 4-(5-methyl-2,5-diazabicyclo[2.2.1]heptan-2-yl)-1H-benzo[d]imidazole (400 mg, 58.9%) as a brown oil. LC-MS m/z: 229.2 [M+H]⁺. Purity (254 nm): 88.04%; t_{R}=1.26 min.

Following general procedure C, 4-(5-methyl-2,5-diazabicyclo[2.2.1]heptan-2-yl)-1H-benzo[d]imidazole (200 mg, 0.88 mmol) and 3-methylbutan-1-amine (76.3 mg, 0.88 mmol) afforded the title compound (89.7 mg, 30%) as a yellow solid. ¹H NMR (500 MHz, CDCl₃) δ 8.41 (s, 1H), 7.33-7.31 (m, 1H), 7.20 (t, J=8.5 Hz, 1H), 7.19-7.13 (m, 1H), 6.38 (d, J=7.6 Hz, 1H), 5.61 (s, 1H), 4.21 (s, 1H), 3.87 (q, J=10.4 Hz, 2H), 3.47 (s, 3H), 3.20 (s, 1H), 2.73 (s, 3H), 2.38-2.22 (m, 2H), 1.79-1.72 (m, 1H), 1.62-1.56 (m, 2H), 0.98 (d, J=6.4 Hz, 6H). LC-MS m/z: 342.0 [M+H]⁺. HPLC Purity (214 nm): >99%; t_{R}=6.07 min.

### Example 160—N-iso-Pentyl-4-(6-methyl-3,6-diazabicyclo[3.1.1]-heptan-3-yl)-1H-benzo[d]imidazole-1-carboxamide

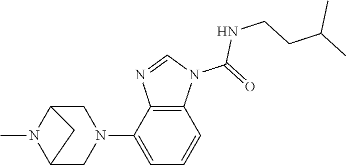 [SMILES: CC(C)CCNC(=O)n1cnc2c(N3CC4CC(C3)N4C)cccc21]

Following general procedure E, 3-fluoro-2-nitroaniline (468 mg, 3.0 mmol) and 6-methyl-3,6-diazabicyclo[3.1.1]heptane (438 mg, 3 mmol) afforded crude 3-(6-methyl-3,6-diazabicyclo-[3.1.1]heptan-3-yl)-2-nitroaniline (250 mg) as a yellow solid. LC-MS m/z: 249.1 [M+H]⁺. Purity (214 nm): 86.4%; t_{R}=1.66 min.

Following general procedure F, 3-(6-methyl-3,6-diazabicyclo[3.1.1]heptan-3-yl)-2-nitroaniline (248 mg, 1.0 mmol) afforded crude 3-(6-methyl-3,6-diazabicyclo[3.1.1]heptan-3-yl)benzene-1,2-diamine (220 mg) as a yellow oil. LC-MS m/z: 219.2 [M+H]⁺. Purity (214 nm): 86.4%; t_{R}=1.33 min.

Following general procedure G (method B), 3-(6-methyl-3,6-diazabicyclo[3.1.1]heptan-3-yl)benzene-1,2-diamine (218 mg, 1.0 mmol) afforded crude 3-(1H-benzo[d]imidazol-4-yl)-6-methyl-3,6-diazabicyclo[3.1.1]-heptane (200 mg) as a yellow oil. LC-MS m/z: 229.2 [M+H]⁺. Purity (214 nm): 76.4%; t_{R}=1.27 min.

Following general procedure C, 3-(1H-benzo[d]imidazol-4-yl)-6-methyl-3,6-diazabicyclo[3.1.1]heptane (114 mg, 0.5 mmol) and 3-methylbutan-1-amine (51.0 mg, 0.5 mmol) afforded the title compound (12.4 mg, 7.3%) as a white solid. ¹H NMR (400 MHz, CDCl₃) δ 8.58 (s, 1H), 7.60 (bs, 1H), 7.52 (d, J=7.8 Hz, 1H), 7.28 (bs 1H), 6.32 (d, J=7.8 Hz, 1H), 4.77 (bs, 2H), 3.75 (d, J=11.4 Hz, 2H), 3.53 (bs, 2H), 3.46 (d, J=8.9 Hz, 2H), 2.91 (bs, 1H), 2.46-2.39 (m, 4H), 1.78-1.67 (m, 1H), 1.60 (q, J=7.1 Hz, 2H), 0.98 (d, J=6.2 Hz, 6H). LC-MS m/z: 342.3 [M+H]⁺. HPLC Purity (214 nm): 98.28%; t_{R}=5.20 min.

### Example 161—N-iso-Pentyl-4-(8-methyl-3,8-diazabicyclo[3.2.1]-octan-3-yl)-1H-benzo[d]imidazole-1-carboxamide

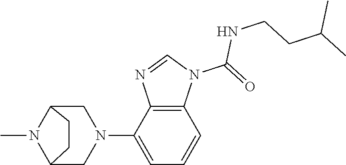 [SMILES: CC(C)CCNC(=O)n1cnc2c(N3CC4CCC(C3)N4C)cccc21]

Following general procedure E, 3-fluoro-2-nitroaniline (552 mg, 3.5 mmol) and 8-methyl-3,8-diazabicyclo[3.2.1]octane (605 mg, 3.5 mmol) afforded 3-(8-methyl-3,8-diazabicyclo[3.2.1]octan-3-yl)-2-nitroaniline (734 mg, 78.9%) as a red solid. LC-MS m/z: 263.2 [M+H]⁺. Purity (214 nm): 31.55%; t_{R}=1.41 min.

Following general procedure F, 3-(8-methyl-3,8-diazabicyclo[3.2.1]octan-3-yl)-2-nitroaniline (734 mg, 2.8 mmol) afforded 3-(8-methyl-3,8-diazabicyclo[3.2.1]octan-3-yl)benzene-1,2-diamine (404 mg, 61.9%) as a red solid. LC-MS m/z: 233.2 [M+H]⁺. Purity (214 nm): 99.81%; t_{R}=0.82 min.

Following general procedure G (method B), 3-(8-methyl-3,8-diazabicyclo[3.2.1]octan-3-yl)benzene-1,2-diamine (404 mg, 1.74 mmol) afforded 4-(8-methyl-3,8-diazabicyclo[3.2.1]octan-3-yl)-1H-benzo[d]imidazole (412 mg, 97.6%) as a red solid. LC-MS m/z: 243.2 [M+H]⁺. Purity (214 nm): 98.51%; t_{R}=1.33 min.

Following general procedure C, 4-(8-methyl-3,8-diazabicyclo[3.2.1]-octan-3-yl)-1H-benzo[d]imidazole (242 mg, 1 mmol) and iso-butylamine (87 mg, 1 mmol) afforded the title compound (122.2 mg, 34.4%) as a white solid. ¹H NMR (400 MHz, CDCl₃) δ 8.34 (s, 1H), 7.24 (d, J=8.0 Hz, 1H), 7.18 (d, J=8.0 Hz, 1H), 6.60 (d, J=7.8 Hz, 1H), 5.81 (t, J=4.8 Hz, 1H), 4.11 (dd, J=11.5, 2.4 Hz, 2H), 3.53 (dd, J=14.7, 5.9 Hz, 2H), 3.34 (bs, 2H), 3.21 (d, J=11.2 Hz, 2H), 2.41 (s, 3H), 2.18-2.03 (m, 4H), 1.74 (sep, J=6.8 Hz, 1H), 1.59 (dd, J=14.8, 7.1 Hz, 2H), 0.98 (d, J=6.6 Hz, 6H). LC-MS m/z: 356.1 [M+H]⁺. HPLC Purity (214 nm): 96.5%; t_{R}=6.72 min.

### Example 162—N-iso-Pentyl-4-(4-methyl-2-oxopiperazin-1-yl)-1H-benzo[d]imidazole-1-carboxamide

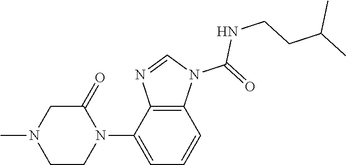 [SMILES: CC(C)CCNC(=O)n1cnc2c(N3CCN(C)CC3=O)cccc21]

To a solution of 4-bromo-1-((2-(trimethylsilyl)ethoxy)methyl)-1H-benzo[d]imidazole (543 mg, 1.67 mmol) in 1,4-dioxane (10 mL) was added K₂CO₃ (461 g, 3.34 mmol), CuI (20 mg, 0.17 mmol) and N,N′-dimethyl-1,2-ethanediamine. The mixture was stirred at 110° C. overnight. The mixture was filtered, concentrated and purified by silica gel column chromatography (DCM:MeOH=10:1) to give tert-butyl-3-oxo-4-(1-((2-(trimethylsilyl)ethoxy)methyl)-1H-benzo[d]imidazol-4-yl)piperazine-1-carboxylate (854 mg, 88.5%) as a red solid. LC-MS m/z: 447.2 [M+H]⁺. Purity (214 nm): 77.99%; t_{R}=1.90 min.

To a solution of tert-butyl 3-oxo-4-(1-((2-(trimethylsilyl)ethoxy)methyl)-1H-benzo[d]imidazol-4-yl)piperazine-1-carboxylate (854 mg, 1.91 mmol) in MeOH (5 mL) was added 1,4-dioxane HCl (10 ml) solution and the mixture was stirred at RT for 2 h and concentrated to afford 1-(1-((2-(trimethylsilyl)ethoxy)-methyl)-1H-benzo[d]imidazol-4-yl)piperazin-2-one (500 mg, 75.4%) as a white solid. LC-MS m/z: 347.1 [M+H]⁺. Purity (214 nm): 90.31%; t_{R}=1.48 min.

To a solution of 1-(1-((2-(trimethylsilyl)ethoxy)methyl)-1H-benzo[d]imidazol-4-yl)piperazin-2-one (500 g, 1.44 mmol) in MeOH (10 mL) was added 37% CH₂O (166 mg, 2.02 mmol). The mixture was stirred at RT for 30 min, NaBH₃CN (272 mg, 4.32 mmol) was added and the resulting mixture was stirred at RT overnight. The mixture was concentrated and purified by silica gel column chromatography (DCM:MeOH=10:1) to give 4-methyl-1-(1-((2-(trimethylsilyl)ethoxy)methyl)-1H-benzo[d]imidazol-4-yl)piperazin-2-one (379 mg, 72.9%) as a white solid. LC-MS m/z: 361.2 [M+H]⁺. Purity (214 nm): 75.52%; t_{R}=1.66 min.

A solution of 4-methyl-1-(1-((2-(trimethylsilyl)ethoxy)methyl)-1H-benzo[d]imidazol-4-yl)piperazin-2-one (379 mg, 1.05 mmol) in TBAF was stirred at 60° C. overnight. The mixture was concentrated and purified by silica gel column chromatography (DCM:MeOH=10:1) to afford 1-(1H-benzo[d]imidazol-4-yl)-4-methylpiperazin-2-one (230 mg, 67.9%) as a white solid. LC-MS m/z: 231.1 [M+H]⁺. Purity (214 nm): 71.45%; t_{R}=1.07 min.

Following general procedure C, 1-(1H-benzo[d]imidazol-4-yl)-4-methylpiperazin-2-one (230 mg, 1 mmol) and iso-butylamine (87 mg, 1 mmol) afforded the title compound (19.9 mg, 5.8%) as a white solid. ¹H NMR (400 MHz, CDCl₃) δ 8.20 (s, 1H), 8.04 (d, J=8.0 Hz, 1H), 7.42-7.35 (m, 2H), 7.21 (d, J=7.6 Hz, 1H), 3.88 (bs, 2H), 3.56 (bs, 2H), 3.23 (dd, J=13.8, 6.3 Hz, 2H), 3.08 (bs, 2H), 2.59 (s, 3H), 1.70-1.61 (m, 1H), 1.45 (q, J=7.6 Hz, 2H), 0.95 (d, J=6.4 Hz, 6H). LC-MS m/z: 326.3 [M+H]⁺. HPLC Purity (214 nm): 100%; t_{R}=6.10 min.

### Example 163—N-iso-Pentyl-4-(4-methyl-3-oxopiperazin-1-yl)-1H-benzo[d]imidazole-1-carboxamide

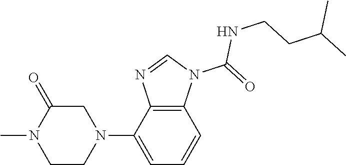 [SMILES: CC(C)CCNC(=O)n1cnc2c(N3CCN(C)C(=O)C3)cccc21]

Following general procedure E, 1-methylpiperazin-2-one (1.0 g, 8.8 mmol) and 3-fluoro-2-nitroaniline (1.4 g, 8.8 mmol) afforded 4-(3-amino-2-nitrophenyl)-1-methylpiperazin-2-one (361 mg, 16.4%) as a red solid. LC-MS m/z: 251.1 [M+H]⁺. Purity (214 nm): 28.05%; t_{R}=1.34 min.

Following general procedure F, 4-(3-amino-2-nitrophenyl)-1-methylpiperazin-2-one (361 mg, 1.4 mmol) afforded 4-(2,3-diaminophenyl)-1-methylpiperazin-2-one (307 mg, 96.5%) as a yellow solid. LC-MS m/z: 233.2 [M+H]⁺. Purity (214 nm): 67.77%; t_{R}=1.20 min.

Following general procedure G (method B), 4-(2,3-diaminophenyl)-1-methylpiperazin-2-one (307 mg, 1.4 mmol) afforded crude 4-(1H-benzo[d]imidazol-4-yl)-1-methylpiperazin-2-one (335 mg, 80.5%) as a red oil. LC-MS m/z: 231.1 [M+H]⁺. Purity (214 nm): 77.59%; t_{R}=1.21 min.

Following general procedure C, 4-(1H-benzo[d]imidazol-4-yl)-1-methylpiperazin-2-one (335 mg, 1 mmol) and iso-butylamine (87 mg, 1 mmol) afforded the title compound (119.2 mg, 23.8%) as a white solid. ¹H NMR (400 MHz, CDCl₃) δ 8.38 (s, 1H), 7.40 (d, J=8.1 Hz, 1H), 7.29 (d, J=8.1 Hz, 1H), 6.64 (d, J=7.9 Hz, 1H), 6.24 (t, J=5.0 Hz, 1H), 4.05 (s, 2H), 3.98 (t, J=5.6 Hz, 2H), 3.62-3.53 (m, 4H), 3.03 (s, 3H), 1.70-1.62 (m, 1H), 1.60 (dd, J=14.8, 7.1 Hz, 2H), 0.99 (d, J=6.6 Hz, 6H). LC-MS m/z: 344.3 [M+H]⁺. HPLC Purity (214 nm): 100%; t_{R}=7.80 min.

### Example 164—N-iso-Pentyl-4-(2-(trifluoromethyl)-aziridin-1-yl)-1H-benzo[d]imidazole-1-carboxamide

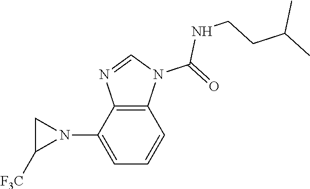 [SMILES: CC(C)CCNC(=O)n1cnc2c(N3CC3C(F)(F)F)cccc21]

Following general procedure G (method B), 3-nitrobenzene-1,2-diamine (1.0 g, 6.53 mmol) afforded 4-nitro-1H-benzo[d]imidazole (900 mg, 90.1%) as a yellow solid. LC-MS m/z: 164.1 [M+H]⁺. Purity (214 nm): 99.0%; t_{R}=1.31 min.

To a solution of 4-nitro-1H-benzo[d]imidazole (500 mg, 3.06 mmol) was added NaH (245 mg, 6.12 mmol) and the mixture was stirred at ° C. for 30 min, then SEMCl (763 mg. 4.6 mmol) was added and the mixture was stirred at RT for 2 h. Water was added slowly, and then extracted with EA (100 mL×2). The combined organic layers were washed with water (20 mL×3) and brine (50 mL), dried over Na₂SO₄, filtered, concentrated and purified by silica gel column chromatography (PE:EA=3:1) to give 4-nitro-1-((2-(trimethylsilyl)ethoxy)methyl)-1H-benzo[d]imidazole (100 mg, 11.0%) as a brown oil. LC-MS m/z: 294.0 [M+H]⁺. Purity (214 nm): 95%; t_{R}=2.01 min.

Following general procedure F, 4-nitro-1-((2-(trimethylsilyl)ethoxy)methyl)-1H-benzo[d]imidazole (100 mg, 0.34 mmol) afforded 1-((2-(trimethylsilyl)ethoxy)methyl)-1H-benzo[d]imidazol-4-amine (70 mg, 78.6%) as a black solid. LC-MS m/z: 264.0 [M+H]⁺. Purity (214 nm): 97%; t_{R}=1.74 min.

A suspension of 1-((2-(trimethylsilyl)ethoxy)methyl)-1H-benzo[d]imidazol-4-amine (70 mg, 0.26 mmol) and Na₂CO₃ (148 mg, 0.32 mmol) in DCM (2.5 mL) was stirred at RT for 1 min, and then (4-fluorophenyl)(3,3,3-trifluoroprop-1-en-2-yl)iodonium, trifluoromethane sulfonate (148 mg, 0.32 mmol) was added. The solution was stirred at RT for 1 h, concentrated and purified by silica gel column chromatography (PE:EA=3:1) to give 4-(2-(trifluoromethyl)-aziridin-1-yl)-1-((2-(trimethylsilyl)ethoxy)methyl)-1H-benzo[d]imidazole (80 mg, 86.8%) as a brown oil. LC-MS m/z: 358.1 [M+H]⁺. Purity (214 nm): 90%; t_{R}=1.49 min.

A stirred solution of 4-(2-(trifluoromethyl)aziridin-1-yl)-1-((2-(trimethylsilyl)ethoxy) methyl)-1H-benzo[d]imidazole (80 mg, 0.22 mmol) in TBAF (4 mL) was stirred at RT for 3 days. The reaction mixture was concentrated and the residue was purified by silica gel column chromatography (100% EA) to give 4-(2-(trifluoromethyl)aziridin-1-yl)-1H-benzo[d]imidazole (18.0 mg, 36.0%) as a white solid. LC-MS m/z: 228.0 [M+H]⁺. Purity (214 nm): 99%; t_{R}=1.45 min.

Following general procedure C, 4-(2-(trifluoromethyl)aziridin-1-yl)-1H-benzo[d]imidazole (13 mg, 0.057 mmol) and 3-methylbutan-1-amine (10 mg, 0.114 mmol) afforded the title compound (13.0 mg, 68.6%) as a white solid. ¹H NMR (400 MHz, CDCl₃) δ 8.36 (s, 1H), 7.47 (dd, J=8.0 Hz, 0.4 Hz, 1H), 7.30 (t, J=8.0 Hz, 1H), 6.97 (d, J=7.2 Hz, 1H), 5.70 (s, 1H), 3.56-3.51 (m, 2H), 3.02-2.98 (m, 1H), 2.87 (d, J=3.2 Hz, 1H), 2.53 (d, J=6.0 Hz, 1H), 1.72 (sep, J=6.8 Hz, 1H), 1.62-1.57 (m, 2H), 0.99 (d, J=6.4 Hz, 6H). LC-MS m/z: 341.0 [M+H]⁺. LC-MS m/z: 341.1 [M+H]⁺. HPLC Purity (214 nm): 99%; t_{R}=9.71 min.

### Example 165—4-(3-Cyano-3-methylazetidin-1-yl)-N-isopentyl-1H-benzo[d]imidazole-1-carboxamide

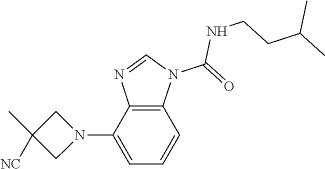 [SMILES: CC(C)CCNC(=O)n1cnc2c(N3CC(C)(C#N)C3)cccc21]

Following general procedure E, 3-fluoro-2-nitroaniline (500 mg, 3.21 mmol) and 3-methylazetidine-3-carbonitrile hydrochloride (719 mg, 5.45 mmol) afforded 1-(3-amino-2-nitrophenyl)-3-methylazetidine-3-carbonitrile (700 mg, 94%) as an orange solid. LC-MS m/z: 233.1 [M+H]⁺. Purity (214 nm): 99%; t_{R}=1.35 min.

Following general procedure F, 1-(3-amino-2-nitrophenyl)-3-methylazetidine-3-carbonitrile (700 mg, 3.02 mmol) afforded 1-(2,3-diaminophenyl)-3-methylazetidine-3-carbonitrile (600 mg, 98%) as an orange solid. LC-MS m/z: 203.1 [M+H]⁺. Purity (214 nm): 95%; t_{R}=1.23 min.

Following general procedure G (method B), 1-(2,3-diaminophenyl)-3-methylazetidine-3-carbonitrile (600 mg, 2.97 mmol) afforded 1-(1H-benzo[d]imidazol-4-yl)-3-methylazetidine-3-carbonitrile (570 mg, 91%) as a purple oil. LC-MS m/z: 213.1 [M+H]⁺. Purity (214 nm): 91%; t_{R}=1.57 min.

Following general procedure C, 1-(1H-benzo[d]imidazol-4-yl)-3-methylazetidine-3-carbonitrile (180 mg, 0.85 mmol) and 3-methylbutan-1-amine (110 mg, 1.27 mmol) afforded the title compound (42.7 mg, 16%) as a yellowish solid. ¹H NMR (400 MHz, CDCl₃) δ 8.26 (s, 1H), 7.24 (t, J=8.0 Hz, 1H), 7.13 (d, J=7.7 Hz, 1H), 6.25 (d, J=7.4 Hz, 1H), 5.83 (s, 1H), 4.54 (d, J=7.8 Hz, 2H), 4.19 (d, J=7.8 Hz, 2H), 3.52 (dt, J=7.5, 5.8 Hz, 2H), 1.81 (s, 3H), 1.71 (sept, J=6.4 Hz, 1H), 1.58 (q, J=7.2 Hz, 2H), 0.98 (d, J=6.6 Hz, 6H). LC-MS m/z: 326.3 [M+H]⁺. HPLC Purity (214 nm): 99%; t_{R}=9.10 min.

### Example 166—4-((1R,4R)-2-Oxa-5-azabicyclo[2.2.1]heptan-5-yl)-N-iso-pentyl-1H-benzo[d]imidazole-1-carboxamide

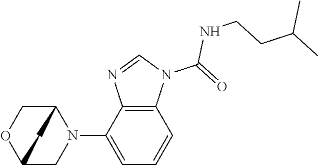

Following general procedure E, 3-fluoro-2-nitroaniline (312 mg, 2.0 mmol) and (1S,4S)-2-oxa-5-azabicyclo[2.2.1]heptane hydrochloride (270 mg, 2.0 mmol) afforded 3-((1S,4S)-2-oxa-5-azabicyclo[2.2.1]-heptan-5-yl)-2-nitroaniline (0.3 g, 63.8%) as a pale yellow solid. LC-MS m/z: 236.1 [M+H]⁺.

Following general procedure F, 3-((1S,4S)-2-oxa-5-azabicyclo[2.2.1]heptan-5-yl)-2-nitroaniline (300 mg, 1.28 mmol) afforded 3-((1S,4S)-2-oxa-5-azabicyclo[2.2.1]heptan-5-yl)benzene-1,2-diamine (300 mg, 100%) as a pale yellow oil. LC-MS m/z: 206.1 [M+H]⁺.

Following general procedure G (method B), 3-((1S,4S)-2-oxa-5-azabicyclo[2.2.1]heptan-5-yl)benzene-1,2-diamine (300 mg, 1.5 mmol) afforded crude (1S,4S)-5-(1H-benzo[d]imidazol-4-yl)-2-oxa-5-azabicyclo[2.2.1]heptane (215 mg, 68.3%) as a pale yellow oil which was used directly in the next step. LC-MS m/z: 216.1 [M+H]⁺.

Following general procedure C, (1S,4S)-5-(1H-benzo[d]imidazol-4-yl)-2-oxa-5-azabicyclo[2.2.1]heptane (215 mg, 1.0 mmol) and 3-methylbutan-1-amine (78 mg, 1 mmol) afforded the title compound (33.7 mg, 10.3%) as a white solid. ¹H NMR (400 MHz, CDCl₃) δ 7.22 (t, J=8.0 Hz, 1H), 6.98 (d, J=8.0 Hz, 1H), 6.38 (d, J=8.0 Hz, 1H), 5.80 (bs, 1H), 5.67 (s, 1H), 4.68 (s, 1H), 3.95 (s, 2H), 3.77 (d, J=9.6 Hz, 1H), 3.53 (q, J=7.6 Hz, 2H), 3.45 (d, J=9.6 Hz, 1H), 2.06 (d, J=2.0 Hz, 1H), 2.05 (d, J=2.0 Hz, 1H), 1.78-1.64 (m, 2H), 1.58 (q, J=6.9 Hz, 2H), 0.99 (d, J=6.6 Hz, 6H). LC-MS m/z: 329.1 [M+H]⁺. HPLC Purity (214 nm): 100%; t_{R}=9.32 min.

### Example 167—4-((2S,6R)-2,6-Dimethylmorpholino)-N-iso-pentyl-1H-benzo[d]imidazole-1-carboxamide

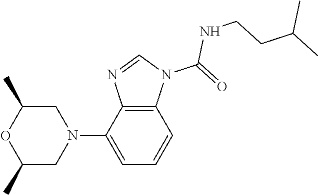 [SMILES: CC(C)CCNC(=O)n1cnc2c(N3C[C@@H](C)O[C@@H](C)C3)cccc21]

Following general procedure H, 4-bromo-1-((2-(trimethylsilyl)ethoxy)methyl)-1H-benzo[d]imidazole (1.5 g, 4.59 mmol) and (2S,6R)-2,6-dimethylmorpholine (791.3 mg, 6.9 mmol) afforded (2S,6R)-2,6-dimethyl-4-(1-((2-(trimethylsilyl)ethoxy)methyl)-1H-benzo[d]imidazol-4-yl)morpholine (1.0 g, 27.9%) as a dark brown oil. LC-MS m/z: 362.2 [M+H]⁺. Purity (254 nm): 46.2%; t_{R}=1.47 min.

To a solution of (2S,6R)-2,6-dimethyl-4-(1-((2-(trimethylsilyl)ethoxy)methyl)-1H-benzo[d]imidazole-4-yl)morpholine (1.0 g, 1.3 mmol) in THF (50 mL) was added n-BuLi (327.8 mg, 5.1 mmol). The solution was stirred at 60° C. for 2 h, then cooled, concentrated and purified by Prep-HPLC to afford (2S,6R)-4-(1H-benzo[d]imidazol-4-yl)-2,6-dimethylmorpholine (110 mg, 36.6%) as a white solid. LC-MS m/z: 232.1 [M+H]⁺. Purity (254 nm): 100.0%; t_{R}=1.10 min.

Following general procedure C, (2S,6R)-4-(1H-benzo[d]imidazol-4-yl)-2,6-dimethylmorpholine (110.0 mg, 0.5 mmol) and 3-methylbutan-1-amine (60.9 mg, 0.7 mmol) afforded the title compound (42.0 mg, 28.2%) as a white solid. ¹H NMR (400 MHz, DMSO) δ 8.55 (s, 1H), 8.50 (t, J=4.8 Hz, 1H), 7.57 (d, J=8.2 Hz, 1H), 7.20 (t, J=8.0 Hz, 1H), 6.66 (d, J=8.0 Hz, 1H), 4.14 (d, J=11.7 Hz, 2H), 3.81 (bs, 2H), 2.50 (bs, 2H), 2.37 (t, J=11.2 Hz, 2H), 1.70-1.62 (m, 1H), 1.49 (q, J=6.9 Hz, 2H), 1.16 (d, J=6.2 Hz, 6H), 0.93 (d, J=6.6 Hz, 6H). LC-MS m/z: 345.2 [M+H]⁺. HPLC Purity (214 nm): 77.87%; t_{R}=8.96 min.

### Example 168—4-((2S,6S)-2,6-Dimethylmorpholino)-N-iso-pentyl-1H-benzo[d]imidazole-1-carboxamide

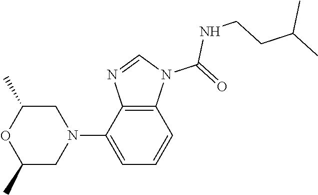 [SMILES: CC(C)CCNC(=O)n1cnc2c(N3C[C@@H](C)O[C@H](C)C3)cccc21]

Following general procedure E, 3-fluoro-2-nitrobenzenamine (1.2 g, 7.7 mmol) and (2S,6S)-2,6-dimethylmorpholine (737.2 mg, 6.4 mmol) afforded 3-((2S,6S)-2,6-dimethylmorpholino)-2-nitrobenzenamine (2.3 g, 93.1%) as a yellow solid. LC-MS m/z: 252.2 [M+H]⁺. Purity (254 nm): 78.0%; t_{R}=1.84 min.

Following general procedure F, 3-((2S,6S)-2,6-dimethylmorpholino)-2-nitrobenzenamine (2.3 g, 7.1 mmol) afforded 3-((2S,6S)-2,6-dimethylmorpholino)benzene-1,2-diamine (1.9 g, 89.6%) as a brown oil. LC-MS m/z: 222.2 [M+H]⁺. Purity (214 nm): 74%; t_{R}=1.42 min.

Following general procedure G (method B), 3-((2S,6S)-2,6-dimethylmorpholino) benzene-1,2-diamine (500 mg, 1.7 mmol) afforded (2S,6S)-4-(1H-benzo[d]imidazol-4-yl)-2,6-dimethylmorpholine (300.0 mg, 76.4%) as a yellow oil. LC-MS m/z: 232.2 [M+H]⁺. Purity (214 nm): 89.1%; t_{R}=1.60 min.

Following general procedure C, (2S,6S)-4-(1H-benzo[d]imidazol-4-yl)-2,6-dimethylmorpholine (200 mg, 0.9 mmol) and 3-methylbutan-1-amine (95.7 mg, 1.1 mmol) afforded the title compound (25.0 mg, 14.9%) as a white solid. ¹H NMR (400 MHz, CDCl₃) δ 8.33 (s, 1H), 7.31-7.24 (m, 2H), 6.69 (dd, J=7.2, 1.6 Hz, 1H), 5.74 (bs, 1H), 4.32-4.25 (m, 2H), 3.61-3.51 (m, 4H), 3.27 (dd, J=11.6, 6.0 Hz, 2H), 1.74-1.64 (m, 1H), 1.59 (q, J=7.1 Hz, 2H), 1.39 (d, J=6.5 Hz, 6H), 0.99 (d, J=6.6 Hz, 6H). LC-MS m/z: 345.2 [M+H]⁺. HPLC Purity (214 nm): 100.0%; t_{R}=8.86 min.

### Example 169—(S)—N-iso-Pentyl-4-(3-methylmorpholino)-1H-benzo[d]imidazole-1-carboxamide

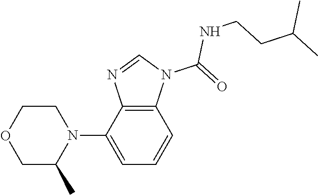 [SMILES: CC(C)CCNC(=O)n1cnc2c(N3CCOC[C@@H]3C)cccc21]

Following general procedure E, 3-fluoro-2-nitroaniline (500 mg, 3.2 mmol) and (S)-3-methylmorpholine (486 mg, 4.8 mmol) afforded (S)-3-(3-methylmorpholino)-2-nitroaniline (410 mg, 27%) as an orange semi-solid. LC-MS m/z: 238.1 [M+H]⁺. Purity (214 nm): 100%; t_{R}=1.35 min.

Following general procedure F, (S)-3-(3-methylmorpholino)-2-nitroaniline (410 mg, 1.73 mmol) afforded crude (S)-3-(3-methylmorpholino)benzene-1,2-diamine (400 mg) as a light brown semi-solid. LC-MS m/z: 208.2 [M+H]⁺. Purity (254 nm): 99%; t_{R}=0.41 min.

Following general procedure G (method B), (S)-3-(3-methylmorpholino)benzene-1,2-diamine (400 mg, 1.93 mmol) afforded (S)-4-(1H-benzo[d]imidazol-4-yl)-3-methylmorpholine (320 mg, crude) as an orange solid. LC-MS m/z: 218.1 [M+H]⁺. Purity (214 nm): 100%; t_{R}=1.34 min.

Following general procedure C, (S)-4-(1H-benzo[d]imidazol-4-yl)-3-methyl morpholine (200 mg, 0.92 mmol) and 3-methylbutan-1-amine (80 mg, 0.92 mmol) afforded the title compound (20.6 mg, 6.8%) as a white solid. ¹H NMR (400 MHz, CDCl₃) δ 8.33 (s, 1H), 7.31-7.27 (m, 2H), 6.78-6.68 (m, 1H), 5.75 (bs, 1H), 4.74 (dt, J=10.1, 3.3 Hz, 1H), 4.12-4.02 (m, 2H), 3.93-3.81 (td, J=10.0, 2.8 Hz, 1H), 3.74 (dd, J=11.4, 2.7 Hz, 1H), 3.60-3.48 (m, 3H), 3.25 (dt, J=12.0, 3.0 Hz, 1H), 1.73 (sept, J=6.8 Hz, 1H), 1.61 (q, J=7.6 Hz, 2H), 1.07 (d, J=6.6 Hz, 3H), 0.99 (d, J=6.6 Hz, 6H). LC-MS m/z: 331.2 [M+H]⁺. HPLC Purity (214 nm): 94.61%; t_{R}=7.32 min.

### Example 170—4-(2,2-Dimethylmorpholino)-N-isopentyl-1H-benzo[d]imidazole-1-carboxamide

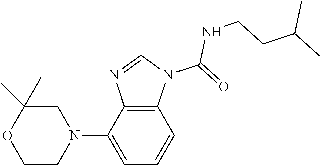 [SMILES: CC(C)CCNC(=O)n1cnc2c(N3CCOC(C)(C)C3)cccc21]

Following general procedure E, 3-fluoro-2-nitroaniline (670 mg, 4.3 mmol) and 2,2-dimethylmorpholine (500 mg, 4.3 mmol) afforded 3-(2,2-dimethylmorpholino)-2-nitroaniline (900 mg, 82.5%) as a red solid. LC-MS m/z: 252.1 [M+H]⁺. Purity (254 nm): 71.92%; t_{R}=1.38 min.

Following general procedure F, 3-(2,2-dimethylmorpholino)-2-nitroaniline (900 mg, 3.6 mmol) afforded crude 3-(2,2-dimethylmorpholino)benzene-1,2-diamine (950 mg, 60%) as a black solid. LC-MS m/z: 222.2 [M+H]⁺. HPLC Purity (214 nm): 58.86%; t_{R}=1.65 min.

Following general procedure G (method B), 3-(2,2-dimethylmorpholino)benzene-1,2-diamine (950 mg, 4.3 mmol) afforded 4-(1H-benzo[d]imidazol-4-yl)-2,2-dimethylmorpholine (710 mg, 71.1%) as a black oil. LC-MS m/z: 232.1 [M+H]⁺. Purity (254 nm): 100.0%; t_{R}=1.81 min.

Following general procedure B, 4-(1H-benzo[d]imidazol-4-yl)-2,2-dimethyl morpholine (200 mg, 0.87 mmol) and 3-methylbutan-1-amine (76 mg, 0.87 mmol) afforded the title compound (38.9 mg, 9.8%) as a white solid. ¹H NMR (400 MHz, DMSO) δ 8.55 (s, 1H), 8.49 (t, J=5.3 Hz, 1H), 7.57 (d, J=8.1 Hz, 1H), 7.20 (t, J=8.1 Hz, 1H), 6.66 (d, J=7.9 Hz, 1H), 3.89-3.74 (m, 2H), 3.30 (bs, 4H), 2.08 (s, 2H), 1.74-1.58 (m, 1H), 1.49 (q, J=7.6 Hz, 2H), 1.29 (s, 6H), 0.93 (d, J=6.6 Hz, 6H). LC-MS m/z: 345.1 [M+H]⁺. HPLC Purity (214 nm): 95.25%; t_{R}=9.15 min.

### Example 171—(S)—N-iso-Pentyl-4-(2-methylmorpholino)-1H-benzo[d]imidazole-1-carboxamide

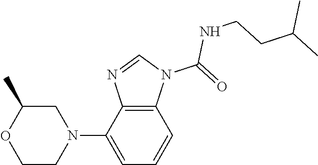 [SMILES: CC(C)CCNC(=O)n1cnc2c(N3CCO[C@@H](C)C3)cccc21]

Following general procedure E, 3-fluoro-2-nitrobenzenamine (500 mg, 3.2 mmol) and (S)-2-methylmorpholine (523 mg, 3.8 mmol) afforded (S)-3-(2-methylmorpholino)-2-nitrobenzenamine as a yellow oil (600 mg, 78%). LC-MS m/z: 238 [M+H]⁺. Purity (254 nm): 89%; t_{R}=1.78 min.

Following general procedure F, (S)-3-(2-methylmorpholino)-2-nitrobenzenamine (600 mg, 2.5 mmol) afforded (S)-3-(2-methylmorpholino)benzene-1,2-diamine (500 mg 98%) as an oil. LC-MS m/z: 208 [M+H]⁺. Purity (214 nm): 81%; t_{R}=1.57 min.

Following general procedure G (method B), (S)-3-(2-methylmorpholino)benzene-1,2-diamine (500 mg, 2.9 mmol) afforded (S)-4-(2-methylmorpholino)-1H-benzo[d]imidazole as a yellow oil (400 mg 50%). LC-MS m/z: 218 [M+H]⁺. Purity (254 nm): 70%; t_{R}=1.52 min.

Following general procedure C, (S)-4-(2-methylmorpholino)-1H-benzo[d]imidazole (400 mg, 1.84 mmol) and 3-methylbutan-1-amine (88.8 mg, 1.84 mmol) afforded the title compound (91 mg 15%) as a white solid. ¹H NMR (400 MHz, CDCl₃) δ 7.30-7.29 (m, 2H), 7.27 (d, J=2.0 Hz, 1H), 6.74 (t, J=7.1 Hz, 1H), 5.74 (bs, 1H), 4.07-3.94 (m, 5H), 3.54 (dd, J=13.7, 6.9 Hz, 2H), 2.97 (t, J=7.1 Hz, 1H), 2.62 (t, J=10.7 Hz, 1H), 1.80-1.72 (m, 1H), 1.61 (bs, 1H), 1.27 (d, J=6.2 Hz, 3H), 1.00 (dd, J=6.5, 2.2 Hz, 6H). LC-MS m/z: 331.1 [M+H]⁺. HPLC Purity (214 nm): 99%; t_{R}=8.53 min.

### Examples 172a and 172b—(S)—N-iso-Pentyl-4-(2-iso-propylmorpholino)-1H-benzo[d]imidazole-1-carboxamide and (R)—N-iso-pentyl-4-(2-iso-propylmorpholino)-1H-benzo[d]imidazole-1-carboxamide

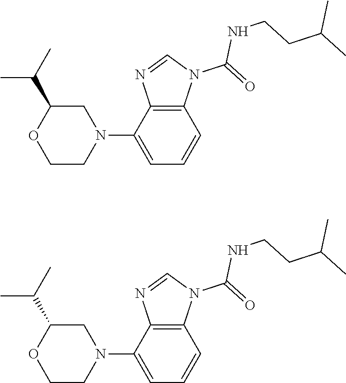

Following general procedure E, 3-fluoro-2-nitroaniline (500 mg, 3.2 mmol) and 2-iso-propylmorpholine (496 mg, 3.8 mmol) afforded 3-(2-iso-propylmorpholino)-2-nitroaniline (800 mg, 94%) as a yellow oil. LC-MS m/z: 266.1 [M+H]⁺. Purity (214 nm): >91%; t_{R}=1.57 min.

Following general procedure F, 3-(2-iso-propylmorpholino)-2-nitroaniline (800 mg, 3.0 mmol) afforded 3-(2-iso-propylmorpholino)benzene-1,2-diamine (700 mg, 99%) as a yellow oil. LC-MS m/z: 236.2 [M+H]⁺. Purity (254 nm): >91%; t_{R}=1.24 min.

Following general procedure G (method B), 3-(2-iso-propylmorpholino)benzene-1,2-diamine (700 mg, 2.98 mmol) afforded 4-(1H-benzo[d]imidazol-4-yl)-2-iso-propylmorpholine (700 mg, 96%) which was purified by chiral-HPLC to afford (S)-4-(1H-benzo[d]imidazol-4-yl)-2-iso-propylmorpholine (270.0 mg, 36.8%) and (R)-4-(1H-benzo[d]imidazol-4-yl)-2-iso-propylmorpholine (290 mg, 39.6%) both as white solids. LC-MS m/z: 246.1 [M+H]⁺. Purity (254 nm): >99%; t_{R}=1.52 min.

Following general procedure C, (S)-4-(1H-benzo[d]imidazol-4-yl)-2-iso-propylmorpholine (100 mg, 0.41 mmol) and 3-methylbutan-1-amine (36 mg, 0.41 mmol) afforded (S)—N-iso-pentyl-4-(2-iso-propylmorpholino)-1H-benzo[d]imidazole-1-carboxamide (26.0 mg, 17.8%) as a colorless oil. ¹H NMR (400 MHz, CDCl₃) δ 8.33 (s, 1H), 7.30 (d, J=6.5 Hz, 2H), 6.74 (t, J=4.4 Hz, 1H), 5.72 (bs, 1H), 4.18-4.03 (m, 3H), 4.02-3.96 (m, 1H), 3.53 (dt, J=16.8, 8.5 Hz, 3H), 2.94 (td, J=11.5, 3.2 Hz, 1H), 2.69 (t, J=11.0 Hz, 1H), 1.88-1.71 (m, 2H), 1.79 (sept, J=6.8 Hz, 1H), 1.75 (sept, J=6.8 Hz, 1H), 1.03 (dd, J=17.7, 6.8 Hz, 6H), 1.01 (d, J=6.8 Hz, 6H). LC-MS m/z: 359.1 [M+H]⁺. HPLC Purity (214 nm): 98.28%; t_{R}=10.19 min.

Following general procedure C, (R)-4-(1H-benzo[d]imidazol-4-yl)-2-iso-propylmorpholine (100 mg, 0.41 mmol) and 3-methylbutan-1-amine (36 mg, 0.41 mmol) afforded (R)—N-iso-pentyl-4-(2-iso-propylmorpholino)-1H-benzo[d]imidazole-1-carboxamide (27.4 mg, 18.7%) as a colorless oil. 1H NMR (400 MHz, CDCl₃) δ 8.33 (s, 1H), 7.31 (d, J=4.0 Hz, 2H), 6.74 (t, J=4.6 Hz, 1H), 5.71 (bs, 1H), 4.16-4.01 (m, 3H), 4.02-3.97 (m, 1H), 3.60-3.49 (m, 3H), 2.94 (td, J=11.6, 3.4 Hz, 1H), 2.69 (t, J=11.0 Hz, 1H), 1.79 (sept, J=6.8 Hz, 1H), 1.75 (sept, J=6.8 Hz, 1H), 1.64-1.48 (m, 2H), 1.01 (dd, J=17.6, 6.6 Hz, 6H), 1.01 (d, J=6.6 Hz, 6H). LC-MS m/z: 359.1 [M+H]⁺. HPLC Purity (214 nm): 95.06%; t_{R}=9.41 min.

### Example 173—N-iso-Pentyl-4-(1,4-oxazepan-4-yl)-1H-benzo[d]imidazole-1-carboxamide

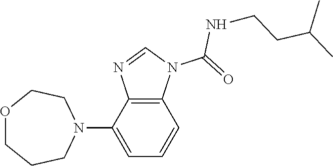 [SMILES: CC(C)CCNC(=O)n1cnc2c(N3CCCOCC3)cccc21]

Following general procedure E, 3-fluoro-2-nitroaniline (312 mg, 2.0 mmol) and 1,4-oxazepane (202 mg, 2.0 mmol) afforded 2-nitro-3-(1,4-oxazepan-4-yl)aniline (0.28 g, 59.1%) as a pale yellow solid. LC-MS m/z: 238.1 [M+H]⁺.

Following general procedure F, 2-nitro-3-(1,4-oxazepan-4-yl)aniline (280 mg, 1.18 mmol) afforded 3-(1,4-oxazepan-4-yl)benzene-1,2-diamine (103 mg, 100%) as a pale yellow oil. LC-MS m/z: 208.1 [M+H]⁺.

Following general procedure G (method B), 3-(1,4-oxazepan-4-yl)benzene-1,2-diamine (103 mg, 0.50 mmol) afforded 4-(1H-benzo[d]imidazol-4-yl)-1,4-oxazepane (86 mg, 80.2%) as a pale yellow oil which was used in the next step. LC-MS m/z: 218.1 [M+H]⁺.

Following general procedure C, 4-(1H-benzo[d]imidazol-4-yl)-1,4-oxazepane (86 mg, 0.40 mmol) and 3-methylbutan-1-amine (78 mg, 1 mmol) afforded the title compound (29.2 mg, 22.3%) as a pale yellow oil. ¹H NMR (400 MHz, CDCl₃) δ 8.26 (s, 1H), 7.21 (t, J=8.0 Hz, 1H), 7.03 (d, J=8.0 Hz, 1H), 6.57 (d, J=8.0 Hz, 1H), 5.79 (bs, 1H), 4.16 (t, J=4.8 Hz, 2H), 3.95-3.89 (m, 4H), 3.76 (t, J=5.6 Hz, 2H), 3.51 (q, J=6.0 Hz, 2H), 2.12 (p, J=6.0 Hz, 2H), 1.76-1.65 (m, 1H), 1.58 (q, J=6.4 Hz, 2H), 0.98 (d, J=6.4 Hz, 6H). LC-MS m/z: 331.1 [M+H]⁺. HPLC Purity (214 nm): 98%; t_{R}=8.14 min.

### Example 174—(S)-4-(2-Benzylmorpholino)-N-iso-pentyl-1H-benzo[d]imidazole-1-carboxamide

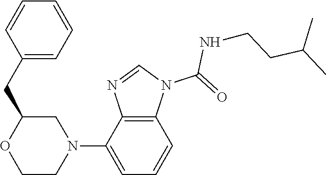 [SMILES: CC(C)CCNC(=O)n1cnc2c(N3CCO[C@@H](Cc4ccccc4)C3)cccc21]

Following general procedure E, 3-fluoro-2-nitrobenzenamine (500 mg, 3.2 mmol) and (S)-2-benzylmorpholine (676 mg, 3.8 mmol) afforded (S)-3-(2-benzylmorpholino)-2-nitrobenzenamine as a yellow oil (600 mg, 60%). LC-MS m/z: 314 [M+H]⁺. Purity (214 nm): 76%; t_{R}=1.91 min.

Following general procedure F, (S)-3-(2-benzylmorpholino)-2-nitrobenzenamine (600 mg, 1.9 mmol) afforded (S)-3-(2-benzylmorpholino)benzene-1,2-diamine (500 mg, 47%) as a oil. LC-MS m/z: 284 [M+H]⁺. Purity (214 nm): 50%; t_{R}=1.88 min.

Following general procedure G (method B), (S)-3-(2-benzylmorpholino)benzene-1,2-diamine (500 mg, 1.77 mmol) afforded (S)-4-(2-benzylmorpholino)-1H-benzo[d]imidazole (400 mg, 50%) as a yellow oil. LC-MS m/z: 294 [M+H]⁺. Purity (254 nm): 67%; t_{R}=1.82 min.

Following general procedure C, (S)-4-(2-benzylmorpholino)-1H-benzo[d]imidazole (400 mg, 1.36 mmol) and 3-methylbutan-1-amine (104.0 mg, 1.2 mmol) afforded the title compound (61 mg 11%) as a white solid. ¹H NMR (400 MHz, CDCl₃) δ 8.31 (s, 1H), 7.36-7.18 (m, 7H), 6.69 (dd, J=6.9, 1.8 Hz, 1H), 5.74 (bs, 1H), 4.17-3.89 (m, 5H), 3.53 (dd, J=14.7, 6.0 Hz, 2H), 3.02-2.80 (m, 3H), 2.71 (t, J=6.4 Hz, 1H), 1.71 (sept, J=6.7 Hz, 1H), 1.58 (q, J=7.6 Hz, 2H), 0.99 (d, J=6.6 Hz, 6H). LC-MS m/z: 407 [M+H]⁺. HPLC Purity (214 nm): >99%; t_{R}=9.62 min.

### Example 175—N-iso-Pentyl-4-(2-oxa-7-azaspiro[3.5]nonan-7-yl)-1H-benzo[d]imidazole-1-carboxamide

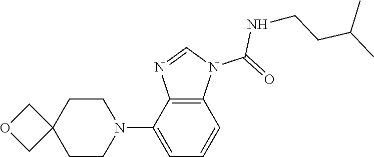 [SMILES: CC(C)CCNC(=O)n1cnc2c(N3CCC4(CC3)COC4)cccc21]

Following general procedure E, 3-fluoro-2-nitroaniline (400 mg, 2.56 mmol) and 2-oxa-7-azaspiro[3.5]-nonane (441 mg, 2.56 mmol) afforded 2-nitro-3-(2-oxa-7-azaspiro[3.5]nonan-7-yl)aniline (400 mg, 59%) as a yellow solid. LC-MS m/z: 264.0 [M+H]⁺. Purity (214 nm): >99%; t_{R}=1.77 min.

Following general procedure F, 2-nitro-3-(2-oxa-7-azaspiro[3.5]nonan-7-yl)aniline (400 mg, 1.5 mmol) afforded 3-(2-oxa-7-azaspiro[3.5]nonan-7-yl)benzene-1,2-diamine (317 mg, 90%) as a yellow solid. LC-MS m/z: 234.2 [M+H]⁺. Purity (254 nm): >99%; t_{R}=0.71 min.

Following general procedure G (method A), 3-(2-oxa-7-azaspiro[3.5]nonan-7-yl)benzene-1,2-diamine (200 mg, 0.86 mmol) afforded 7-(1H-benzo[d]imidazol-4-yl)-2-oxa-7-azaspiro[3.5]nonane (200 mg, 96%) as a yellow solid. LC-MS m/z: 244.2 [M+H]⁺. Purity (254 nm): >88%; t_{R}=0.77 min.

Following general procedure C, 7-(1H-benzo[d]imidazol-4-yl)-2-oxa-7-azaspiro[3.5]nonane (200 mg, 0.82 mmol) and 3-methylbutan-1-amine (72 mg, 0.82 mmol) afforded the title compound (59.9 mg, 23.7%) as a yellow solid. ¹H NMR (400 MHz, CDCl₃) δ 8.35 (s, 1H), 7.33-7.27 (m, 2H), 6.75 (dd, J=4.8, 3.6 Hz, 1H), 5.74 (bs, 1H), 4.51 (s, 4H), 3.63-3.53 (m, 2H), 3.43 (t, J=4.5 Hz, 4H), 2.13 (t, J=5.6 Hz, 4H), 1.79-1.65 (m, 1H), 1.65-1.51 (m, 2H), 0.99 (d, J=6.6 Hz, 6H). LC-MS m/z: 357.0 [M+H]⁺. HPLC Purity (214 nm): 96.37%; t_{R}=7.00 min.

### Example 176—N-iso-Pentyl-4-(2-oxa-7-azaspiro[3.5]nonan-7-yl)-1H-benzo[d]imidazole-1-carboxamide

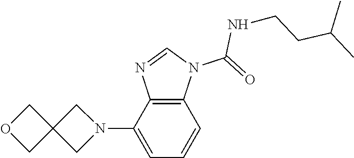 [SMILES: CC(C)CCNC(=O)n1cnc2c(N3CC4(COC4)C3)cccc21]

Following general procedure E, 3-fluoro-2-nitrobenzenamine (500 mg, 3.2 mmol) and 2-oxa-6-azaspiro[3.3]heptane (376 mg, 3.8 mmol) afforded 2-nitro-3-(2-oxa-6-azaspiro[3.3]heptan-6-yl)aniline as a yellow oil (600 mg, 80%). LC-MS m/z: 236[M+H]⁺. Purity (214 nm): 80%; t_{R}=1.43 min.

Following general procedure F, 2-nitro-3-(2-oxa-6-azaspiro[3.3]heptan-6-yl)aniline (600 mg, 2.52 mmol) afforded 3-(2-oxa-6-azaspiro[3.3]heptan-6-yl)benzene-1,2-diamine (500 mg 78%) as an oil. LC-MS m/z: 206 [M+H]⁺. Purity (214 nm): 96%; t_{R}=1.34 min.

Following general procedure G (method A), 3-(2-oxa-6-azaspiro[3.3]heptan-6-yl)benzene-1,2-diamine (100 mg, 0.49 mmol) afforded 6-(1H-benzo[d]imidazol-4-yl)-2-oxa-6-azaspiro[3.3]heptane (80 mg, 76%) as a yellow solid. LC-MS m/z: 216 [M+H]⁺. Purity (214 nm): 58%; t_{R}=1.2 min.

Following general procedure C, 6-(1H-benzo[d]imidazol-4-yl)-2-oxa-6-azaspiro[3.3]heptane (80 mg, 0.37 mmol) and 3-methylbutan-1-amine (29 mg 0.34 mmol) afforded the title compound (56 mg, 46%) as a white solid. ¹H NMR (400 MHz, CDCl₃) δ 8.28 (s, 1H), 7.23 (t, J=4.0 Hz, 1H), 7.05 (d, J=8.8 Hz, 1H), 6.26 (d, J=8.0 Hz, 1H), 5.81 (bs, 1H), 4.87 (s, 4H), 4.39 (s, 4H), 3.51 (dd, J=13.7, 6.6 Hz, 2H), 1.72 (sept, J=6.4 Hz, 1H), 1.58 (dd, J=14.5, 7.2 Hz, 2H), 0.98 (d, J=6.6 Hz, 6H). LC-MS m/z: 329.2 [M+H]⁺. HPLC Purity (214 nm): 99%; t_{R}=8.08 min.

### Example 177—N-iso-Pentyl-4-(3-methoxy-3-methylazetidin-1-yl)-1H-benzo[d]imidazole-1-carboxamide

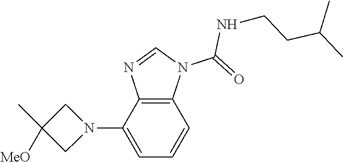 [SMILES: COC1(C)CN(c2cccc3c2ncn3C(=O)NCCC(C)C)C1]

Following general procedure E, 3-fluoro-2-nitrobenzenamine (500 mg, 3.2 mmol) and 3-methoxy-3-methylazetidine (523 mg, 3.8 mmol) afforded 3-(3-methoxy-3-methylazetidin-1-yl)-2-nitrobenzenamine as a yellow oil (600 mg, 80%). LC-MS m/z: 238 [M+H]⁺. Purity (214 nm): 97%; t_{R}=1.78 min.

Following general procedure F, 3-(3-methoxy-3-methylazetidin-1-yl)-2-nitrobenzenamine (600 mg, 2.52 mmol) afforded 3-(3-methoxy-3-methylazetidin-1-yl)benzene-1,2-diamine as an oil (560 mg, 95%). LC-MS m/z: 238 [M+H]⁺. Purity (254 nm): 900%; t_{R}=0.98 min.

Following general procedure G (method B), 3-(3-methoxy-3-methylazetidin-1-yl)benzene-1,2-diamine (560 mg, 2.4 mmol) afforded 4-(3-methoxy-3-methylazetidin-1-yl)-1H-benzo[d]imidazole (400 mg, 47%) as a yellow oil. LC-MS m/z: 218 [M+H]⁺. Purity (254 nm): 95%; t_{R}=1.56 min.

Following general procedure C, 4-(3-methoxy-3-methylazetidin-1-yl)-1H-benzo[d]imidazole (217 mg, 1 mmol) and 3-methylbutan-1-amine (29 mg, 0.34 mmol) afforded the title compound (18 mg 5.4%) as a white solid. ¹H NMR (400 MHz, CDCl₃) δ 8.28 (s, 1H), 7.20 (t, J=8.4 Hz, 1H), 7.05 (d, J=8.4 Hz, 1H), 6.26 (d, J=8.0 Hz, 1H), 5.81 (bs, 1H), 4.16 (d, J=8.4 Hz, 2H), 4.09 (d, J=8.0 Hz, 2H), 3.53-3.48 (m, 2H), 3.30 (s, 3H), 1.72 (sept, J=6.4 Hz, 1H), 1.60 (s, 3H), 1.58 (dd, J=14.5, 7.2 Hz, 2H), 0.98 (d, J=6.6 Hz, 6H). LC-MS m/z: 331 [M+H]⁺. HPLC Purity (214 nm): 95.34%; t_{R}=8.82 min.

### Example 178—6-Cyano-N-(3-cyclopropylpropyl)-4-morpholino-1H-benzo[d]imidazole-1-carboxamide

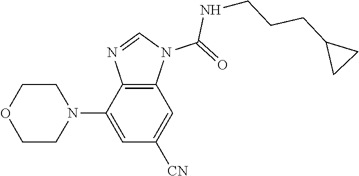 [SMILES: N#Cc1cc(N2CCOCC2)c2ncn(C(=O)NCCCC3CC3)c2c1]

Following general procedure C, 4-morpholino-1H-benzo[d]imidazole-6-carbonitrile (115 mg, 0.50 mmol) and 3-cyclopropylpropan-1-amine (101 mg, 0.75 mmol) afforded the title compound (83.6 mg, 47.5%) as a white solid. ¹H NMR (400 MHz, CDCl₃) δ 8.35 (s, 1H), 7.79 (d, J=1.2 Hz, 1H), 6.90 (s, 1H), 5.74 (bs, 1H), 3.96 (t, J=4.8 Hz, 4H), 3.59-3.54 (m, 6H), 1.83 (p, J=7.6 Hz, 2H), 1.35 (q, J=7.2 Hz, 2H), 0.75-0.71 (m, 1H), 0.51-0.46 (m, 2H), 0.07 (q, J=4.8 Hz, 2H). LC-MS m/z: 354.0 [M+H]⁺. HPLC Purity (214 nm): 99%; t_{R}=8.84 min.

### Example 179—6-Cyano-N-iso-pentyl-4-morpholino-1H-benzo[d]imidazole-1-carboxamide

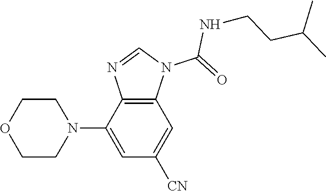 [SMILES: CC(C)CCNC(=O)n1cnc2c(N3CCOCC3)cc(C#N)cc21]

Following general procedure E, 5-bromo-3-fluoro-2-nitroaniline (1.0 g, 4.25 mmol) and morpholine (0.44 g, 5.10 mmol) afforded 5-bromo-3-morpholino-2-nitroaniline (1.0 g, 78.7%) as a red oil. LC-MS m/z: 304.0 [M+H]⁺. Purity (214 nm): 99%; t_{R}=1.68 min.

A suspension of 5-bromo-3-morpholino-2-nitroaniline (1.0 g, 4.14 mmol), Zn (1.07 g, 16.6 mmol) and NH₄Cl (1.09 g, 20 mmol) in EtOH (20 mL) was stirred at 85° C. for 4 h. The mixture was then filtered, concentrated, extracted with DCM (50 mL×2), washed with water (20 mL×2) and brine (20 mL×1), dried over Na₂SO₄, filtered and concentrated to give 5-bromo-3-morpholinobenzene-1,2-diamine (910 mg, 97.8%) as a brown solid. LC-MS m/z: 274.0 [M+H]⁺. Purity (214 nm): 93%; t_{R}=1.56 min.

Following general procedure G (method B), 5-bromo-3-morpholinobenzene-1,2-diamine (500 mg, 1.84 mmol) afforded 4-(6-bromo-1H-benzo[d]imidazol-4-yl)morpholine (460 mg, 88.9%) as a brown oil. LC-MS m/z: 283.9 [M+H]⁺. Purity (214 nm): 90%; t_{R}=1.53 min.

A suspension of 4-(6-bromo-1H-benzo[d]imidazol-4-yl)morpholine (460 mg, 1.63 mmol), ZnCN₂ (861 mg, 7.36 mmol), Pd₂(dba)₃ (298 mg, 0.326 mmol), dppf (903 mg, 0.815 mmol), Zn (32 mg, 0.489 mmol) and Zn(OAc)₂ (89 mg, 0.489 mmol) in anhydrous DMF (5 mL) was stirred at 120° C. for 2 h under N₂. After that, the reaction mixture was concentrated and the residue was purified by silica gel column chromatography (DCM:MeOH=6:1) to give 4-morpholino-1H-benzo[d]imidazole-6-carbonitrile (520 mg, 64.1%) as a brown oil. LC-MS m/z: 229.0 [M+H]⁺. Purity (214 nm): 86%; t_{R}=1.50 min.

Following general procedure C, 4-morpholino-1H-benzo[d]imidazole-6-carbonitrile (100 mg, 0.44 mmol) and 3-methylbutan-1-amine (57 mg, 0.66 mmol) afforded the title compound (28.9 mg, 19.2%) as a white solid. ¹H NMR (400 MHz, CDCl₃) δ 8.35 (s, 1H), 7.80 (s, 1H), 6.90 (s, 1H), 5.70 (t, J=4.4 Hz, 1H), 3.97-3.95 (m, 4H), 3.59-3.52 (m, 6H), 1.73 (sept, J=6.8 Hz, 1H), 1.64-1.58 (m, 2H), 1.00 (d, J=6.4 Hz, 6H). LC-MS m/z: 342.0 [M+H]⁺. HPLC Purity (214 nm): 99%; t_{R}=9.94 min.

### Example 180—6-Cyano-N-iso-pentyl-4-(4-methylpiperazin-1-yl)-1H-benzo[d]imidazole-1-carboxamide

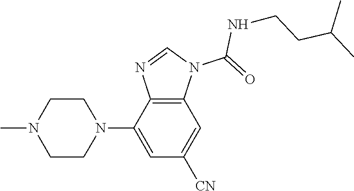 [SMILES: CC(C)CCNC(=O)n1cnc2c(N3CCN(C)CC3)cc(C#N)cc21]

To a solution of 4-bromo-1H-benzo[d]imidazole-6-carbonitrile (1.0 g, 4.50 mmol) in DMF (5 mL) was added NaH (360 mg, 9.01 mmol) at 0° C. under N₂. The mixture was then stirred at 0° C. for 10 min followed by the addition of SEMCl (1.1 g, 6.76 mmol). The resulting mixture was stirred at RT for 1 h, poured into water (100 mL) and extracted with EA (25 mL×2). The combined organic layers were washed with brine (25 mL), dried over Na₂SO₄, filtered and concentrated to give a residue which was purified by silica gel column chromatography (EA:PE=1:2) to give 4-bromo-1-((2-(trimethylsilyl)ethoxy)methyl)-1H-benzo[d]imidazole-6-carbonitrile (1.0 g, 63%) as a pink solid. LC-MS m/z: 351.9 [M+H]⁺. Purity (214 nm): 99%; t_{R}=2.16 min.

Following general procedure H, 4-bromo-1-((2-(trimethylsilyl)ethoxy)methyl)-1H-benzo[d]-imidazole-6-carbonitrile (1 g, 2.92 mmol) and 1-methylpiperazine (437 mg, 4.37 mmol) afforded 4-(4-methylpiperazin-1-yl)-1-((2-(trimethylsilyl)ethoxy)methyl)-1H-benzo[d]imidazole-6-carbonitrile (630 mg, 58%) as an orange solid. LC-MS m/z: 372.0 [M+H]⁺. Purity (214 nm): 68%; t_{R}=1.66 min.

A solution of 4-(4-methylpiperazin-1-yl)-1-((2-(trimethylsilyl)ethoxy)methyl)-1H-benzo[d]imidazole-6-carbonitrile (300 mg, 0.81 mmol) in TFA (3 mL) was stirred at RT for 1 h. The reaction mixture was concentrated and purified by silica gel column chromatography (MeOH:DCM=1:2) to give 4-(4-methylpiperazin-1-yl)-1H-benzo[d]imidazole-6-carbonitrile (100 mg, 51%) as a clear oil. LC-MS m/z: 242.2 [M+H]⁺. Purity (214 nm): 99%; t_{R}=1.30 min.

Following general procedure C, 4-(4-methylpiperazin-1-yl)-1H-benzo[d]imidazole-6-carbonitrile (100 mg, 0.42 mmol) and 3-methylbutan-1-amine (54 mg, 0.62 mmol) afforded the title compound (35.9 mg, 25%) as a yellowish solid. ¹H NMR (400 MHz, CDCl₃) δ 8.35 (s, 1H), 7.75 (d, J=1.2 Hz, 1H), 6.91 (d, J=1.2 Hz, 1H), 5.64 (bs, 1H), 3.63 (bs, 4H), 3.54 (dd, J=7.4, 1.8 Hz, 2H), 2.70 (bs, 4H), 2.41 (s, 3H), 1.76-1.69 (m, 1H), 1.63-1.53 (m, 2H), 1.00 (d, J=6.6 Hz, 6H). LC-MS m/z: 355.2 [M+H]⁺. HPLC Purity (254 nm): 99%; t_{R}=5.74 min.

### Examples 181a and 181b—N-(4-Cyclopropylbutyl)-2-methoxy-6-morpholino-1H-benzo[d]imidazole-1-carboxamide and N-(4-cyclopropylbutyl)-2-methoxy-5-morpholino-1H-benzo[d]imidazole-1-carboxamide

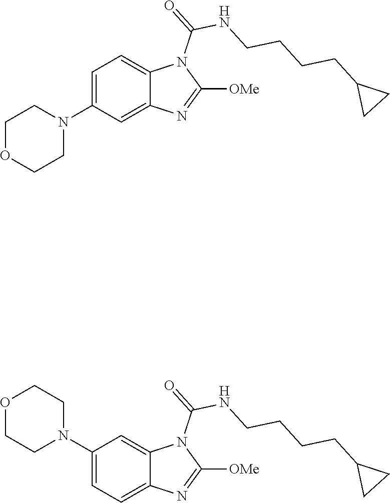 [SMILES: COc1nc2cc(N3CCOCC3)ccc2n1C(=O)NCCCCC1CC1 ; COc1nc2ccc(N3CCOCC3)cc2n1C(=O)NCCCCC1CC1]

Following general procedure C, 4-(2-methoxy-1H-benzo[d]imidazol-6-yl)morpholine (233 mg, 1.0 mmol) and 4-cyclopropylbutan-1-amine (170 mg, 1.5 mmol) afforded N-(4-cyclopropylbutyl)-2-methoxy-6-morpholino-1H-benzo[d]imidazole-1-carboxamide (3.7 mg, 1.0%) and N-(4-cyclopropylbutyl)-2-methoxy-5-morpholino-1H-benzo[d]imidazole-1-carboxamide (27.5 mg, 7.4%) both as white solids.

N-(4-cyclopropylbutyl)-2-methoxy-6-morpholino-1H-benzo[d]imidazole-1-carboxamide ¹H NMR (400 MHz, CDCl₃) δ 8.03 (d, 0.1-8.8 Hz, 1H), 7.06 (d, J=2.4 Hz, 1H), 6.91 (bs, 1H), 6.88 (dd, J=8.8, 2.4 Hz, 2H), 4.30 (s, 3H), 3.88 (t, J=4.8 Hz, 4H), 3.43 (q, J=7.2 Hz, 2H), 3.15 (t, J=4.8 Hz, 4H), 1.69-1.63 (m, 2H), 1.52-1.47 (m, 2H), 1.39-1.33 (m, 2H), 0.69-0.65 (m, 1H), 0.44-0.39 (m, 2H), 0.04-0.00 (m, 2H). LC-MS m/z: 373.1 [M+H]⁺. HPLC Purity (214 nm): 95%; t_{R}=9.47 min.

N-(4-cyclopropylbutyl)-2-methoxy-5-morpholino-1H-benzo[d]imidazole-1-carboxamide ¹H NMR (400 MHz, CDCl₃) δ 7.83 (d, J=2.4 Hz, 1H), 7.38 (d, J=9.6 Hz, 1H), 6.98 (d, J=5.2 Hz, 1H), 6.88 (dd, J=8.8, 2.4 Hz, 1H), 4.30 (s, 3H), 3.88 (t, J=4.8 Hz, 4H), 3.42 (q, J=6.8 Hz, 2H), 3.17 (t, J=4.8 Hz, 4H), 1.69-1.61 (m, 2H), 1.49 (p, J=8.0 Hz, 2H), 1.27 (q, J=7.2 Hz, 2H), 0.69-0.62 (m, 1H), 0.04-0.00 (m, 2H), 0.24-0.36 (m, 2H). LC-MS m/z: 373.1 [M+H]⁺. HPLC Purity (214 nm): 98%; t_{R}=9.41 min.

### Example 182—N-iso-Pentyl-4-(1-methylpiperidin-4-yl)-1H-benzo[d]imidazole-1-carboxamide

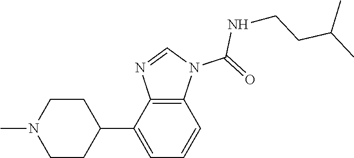 [SMILES: CC(C)CCNC(=O)n1cnc2c(C3CCN(C)CC3)cccc21]

Following general procedure G (method B), 3-bromobenzene-1,2-diamine (2.0 g, 10.7 mmol) afforded crude 4-bromo-1H-benzo[d]imidazole (1.2 g) used directly in the next step. LC-MS m/z: 198.7 [M+H]⁺. Purity (214 nm).

Following general procedure A, 4-bromo-1H-benzo[d]imidazole (1.2 g, 6.1 mmol) and (1-methyl-1,2,3,6-tetrahydropyridin-4-yl)boronic acid (1.3 g, 9.2 mmol) afforded 4-(1-methyl-1,2,3,6-tetrahydropyridin-4-yl)-1H-benzo[d]imidazole (900 mg). LC-MS m/z: 214.7 [M−H]⁻. Purity (214 nm) 90%.

A mixture of 4-(1-methyl-1,2,3,6-tetrahydropyridin-4-yl)-1H-benzo[d]imidazole (900 mg, 4.2 mmol) and Pd/C (10%/o) (300 mg) in 15 mL of MeOH was stirred at RT for 1 h. The reaction mixture was filtered and the filtrated was concentrated to give 4-(1-methylpiperidin-4-yl)-1H-benzo[d]imidazole (550 mg) as a solid. LC-MS m/z: 216.7 [M+H]⁺. Purity (214 nm) 90%.

Following general procedure C, 4-(1-methylpiperidin-4-yl)-1H-benzo[d]imidazole (300 mg, 1.4 mmol) and 3-methylbutan-1-amine (139 mg, 1.7 mmol) afforded the title compound (68.6 mg, 15.00%) as a white solid. ¹H NMR (400 MHz, CDCl₃) δ 8.61 (s, 1H), 7.75 (d, J=8.2 Hz, 1H), 7.34 (t, J=7.9 Hz, 1H), 7.18 (d, J=7.5 Hz, 1H), 6.87 (bs, 1H), 3.54-3.43 (m, 5H), 2.74-2.68 (m, 2H), 2.67 (s, 3H), 2.25 (q, J=11.6 Hz, 2H), 2.06 (d, J=13.5 Hz, 2H), 1.73 (septet, J=6.5 Hz, 1H), 1.60 (q, J=7.6 Hz, 2H), 0.98 (d, J=6.6 Hz, 6H). LC-MS m/z: 329.1 [M+H]⁺. Purity (214 nm): 100%; t_{R}=6.67 min.

### Examples 183a and 183b —2-Methoxy-6-morpholino-N-(3-(1-(trifluoromethyl) cyclopropyl)propyl)-1H benzo[d]imidazole-1-carboxamide and 2-methoxy-5-morpholino-N-(3-(1-(trifluoromethyl)cyclopropyl)propyl)-1H-benzo[d]imidazole-1-carboxamide

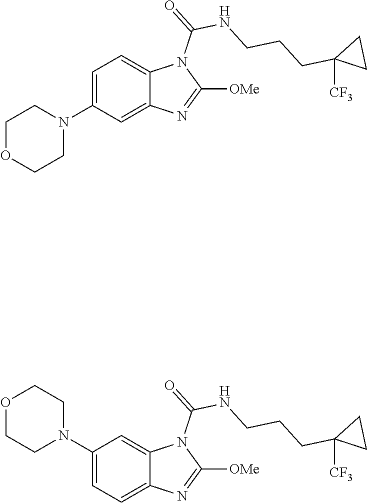 [SMILES: COc1nc2cc(N3CCOCC3)ccc2n1C(=O)NCCCC1(C(F)(F)F)CC1 ; COc1nc2ccc(N3CCOCC3)cc2n1C(=O)NCCCC1(C(F)(F)F)CC1]

Following general procedure C, 4-(2-methoxy-1H-benzo[d]imidazol-6-yl)morpholine (100 mg, 0.43 mmol) and 3-(1-(trifluoromethyl)cyclopropyl)propan-1-amine (144 mg, 0.86 mmol) afforded 2-methoxy-6-morpholino-N-(3-(1-(trifluoromethyl)cyclopropyl)propyl)-1H-benzo[d]imidazole-1-carboxamide (19.1 mg, 10.5%) and 2-methoxy-5-morpholino-N-(3-(1-(trifluoromethyl)cyclopropyl)propyl)-1H-benzo[d]imidazole-1-carboxamide (15 mg, 8.2%) both as white solids.

2-methoxy-6-morpholino-N-(3-(1-(trifluoromethyl)cyclopropyl)propyl)-1H benzo[d]imidazole-1-carboxamide: ¹H NMR (400 MHz, CDCl₃) δ 8.02 (d, J=8.9 Hz, 1H), 7.07 (d, J=2.3 Hz, 1H), 6.92 (bs, 1H), 6.88 (dd, J=8.8, 2.4 Hz, 2H), 4.32 (s, 3H), 3.88 (t, J=4.4 Hz, 4H), 3.42 (dd, J=12.9, 7.0 Hz, 2H), 3.15 (t, J=4.8 Hz, 4H), 1.83-1.78 (m, 2H), 1.67-1.61 (m, 2H), 0.98 (t, J=5.8 Hz, 2H), 0.60 (bs, 2H). LC-MS m/z: 427.7 [M+H]⁺. HPLC Purity (214 nm): 96.15%; t_{R}=7.96 min.

2-methoxy-5-morpholino-N-(3-(1-(trifluoromethyl)cyclopropyl)propyl)-1H-benzo[d]imidazole-1-carboxamide: ¹H NMR (400 MHz, CDCl₃) δ 7.81 (d, J=2.4 Hz, 1H), 7.39 (d, J=8.7 Hz, 1H), 7.01 (bs, 1H), 6.91 (dd, J=8.7, 2.4 Hz, 1H), 4.30 (s, 3H), 3.87 (t, J=4.8 Hz, 4H), 3.42 (dd, J=12.9, 7.0 Hz, 2H), 3.17 (t, J=4.8 Hz, 4H), 1.82-1.78 (m, 2H), 1.68-1.63 (m, 2H), 0.98 (t, J=6.4 Hz, 2H), 0.60 (bs, 2H). LC-MS m/z: 427.7 [M+H]⁺. HPLC Purity (214 nm): >99%; t_{R}=7.42 min.

### Examples 184a and 184b —2-Methoxy-6-morpholino-N-(3-(1-(trifluoromethyl) cyclopropyl)propyl)-1H-benzo[d]imidazole-1-carboxamide and 2-methoxy-5-morpholino-N-(3-(1-(trifluoromethyl)cyclopropyl)propyl)-1H-benzo[d]imidazole-1-carboxamide

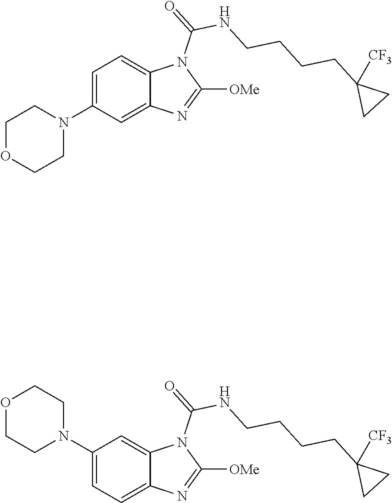 [SMILES: COc1nc2cc(N3CCOCC3)ccc2n1C(=O)NCCCCC1(C(F)(F)F)CC1 ; COc1nc2ccc(N3CCOCC3)cc2n1C(=O)NCCCCC1(C(F)(F)F)CC1]

Following general procedure C, 4-(2-methoxy-1H-benzo[d]imidazol-6-yl)morpholine (100 mg, 0.43 mmol) and 4-(1-(trifluoromethyl)cyclopropyl)butan-1-amine (156 mg, 0.86 mmol) afforded 2-methoxy-6-morpholino-N-(3-(1-(trifluoromethyl)cyclopropyl)propyl)-1H-benzo[d]imidazole-1-carboxamide (7.1 mg, 3.8%) and 2-methoxy-5-morpholino-N-(3-(1-(trifluoromethyl) cyclopropyl)propyl)-1H-benzo[d]imidazole-1-carboxamide (19.4 mg, 10.3%) both as white solids.

2-methoxy-6-morpholino-N-(3-(1-(trifluoromethyl)cyclopropyl)propyl)-1H-benzo[d]imidazole-1-carboxamide: ¹H NMR (400 MHz, CDCl₃) δ 8.02 (d, J=8.9 Hz, 1H), 7.07 (d, J=2.3 Hz, 1H), 6.94 (t, J=5.4 Hz, 1H), 6.89 (dd, J=8.9, 2.4 Hz, 1H), 4.31 (s, 3H), 3.88 (t, J=5.2 Hz, 4H), 3.43 (dd, J=12.6, 6.6 Hz, 2H), 3.15 (t, J=4.8 Hz, 4H), 1.66-1.54 (m, 6H), 0.96 (t, J=5.8 Hz, 2H), 0.57 (bs, 2H). LC-MS m/z: 441.1 [M+H]⁺. HPLC Purity (214 nm): >99%; t_{R}=9.49 min.

2-methoxy-5-morpholino-N-(3-(1-(trifluoromethyl)cyclopropyl)propyl)-1H-benzo[d]imidazole-1-carboxamide: ¹H NMR (400 MHz, CDCl₃) δ 7.82 (d, J=2.4 Hz, 1H), 7.39 (d, J=8.7 Hz, 1H), 7.02 (bs, 1H), 6.91 (dd, J=8.7, 2.4 Hz, 1H), 4.30 (s, 3H), 3.87 (t, J=5.2 Hz, 4H), 3.43 (dd, J=12.7, 6.7 Hz, 2H), 3.19 (t, J=5.2 Hz, 4H), 1.63-1.53 (m, 6H), 0.95 (t, J=6.0 Hz, 2H), 0.57 (bs, 2H). LC-MS m/z: 441.1 [M+H]⁺. HPLC Purity (214 nm): 97.60%; t_{R}=7.97 min.

### Example 185—4-(4-Cyclopropylpiperazin-1-yl)-N-(3,3-dimethylbutyl)-1H-benzo[d]imidazole-1-carboxamide

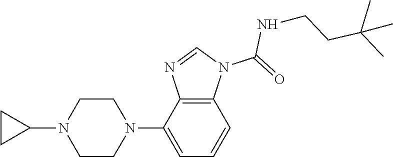 [SMILES: CC(C)(C)CCNC(=O)n1cnc2c(N3CCN(C4CC4)CC3)cccc21]

Following general procedure C, 4-(4-cyclopropylpiperazin-1-yl)-1H-benzo[d]imidazole (240 mg, 1.0 mmol) and 3,3-dimethylbutan-1-amine (121 mg, 1.2 mmol) afforded the title compound (218 mg, 52.5%) as a white solid. ¹H NMR (400 MHz, CDCl₃) δ 8.38 (s, 1H), 7.29-7.26 (m, 2H), 6.73 (d, J=7.7 Hz, 1H), 5.84 (s, 1H), 3.57-3.50 (m, 6H), 3.00 (bs 4H), 1.83 (bs, 1H), 1.67-1.49 (m, 2H), 1.00 (s, 9H), 0.65-0.52 (m, 4H). LC-MS m/z: 370.2 [M+H]⁺. Purity (214 nm): =100%; t_{R}=6.88 min.

### Example 186—N-(4,4-Dimethylpentyl)-4-morpholino-1H-benzo[d]imidazole-1-carboxamide

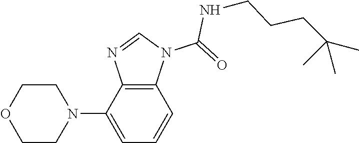 [SMILES: CC(C)(C)CCCNC(=O)n1cnc2c(N3CCOCC3)cccc21]

Following general procedure C, 4-(1H-benzo[d]imidazol-4-yl)-morpholine (200 mg, 0.98 mmol) and 4,4-dimethylpentan-1-amine (149 mg, 0.98 mmol) afforded the title compound 103.7 mg, 30.6%) as a white solid. ¹H NMR (400 MHz, CDCl₃) δ 8.34 (s, 1H), 7.36-7.28 (m, 2H), 6.75 (dd, J=6.4, 2.4 Hz, 1H), 5.78 (bs, 1H), 4.02-3.97 (m, 4H), 3.58-3.54 (m, 4H), 3.50 (q, J=6.0 Hz, 2H), 1.73-1.62 (m, 2H), 1.35-1.25 (m, 2H), 0.92 (s, 9H). LC-MS m/z: 345.2 [M+H]⁺. HPLC Purity (214 nm): 100%; t_{R}=8.99 min.

### Example 187—N-(4-(3-(tert-Butyl)-1H-pyrazol-1-yl)butyl)-2-methoxy-1H-benzo[d]imidazole-1-carboxamide

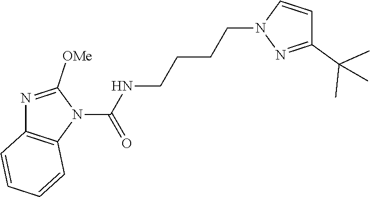 [SMILES: COc1nc2ccccc2n1C(=O)NCCCCn1ccc(C(C)(C)C)n1]

Following general procedure C, 2-methoxy-1H-benzo[d]imidazole (75 mg, 0.51 mmol) and 4-(3-(tert-butyl)-1H-pyrazol-1-yl)butan-1-amine (148 mg, 0.76 mmol) afforded the title compound (39.5 mg, 21%) as a clear oil. ¹H NMR (400 MHz, CDCl₃) δ 8.14 (dd, J=8.8, 2.0 Hz, 1H), 7.50 (dd, J=8.8, 2.0 Hz, 1H), 7.27 (s, 1H), 7.25-7.21 (m, 2H), 7.01 (t, J=5.5 Hz, 1H), 6.07 (d, J=2.1 Hz, 1H), 4.32 (s, 3H), 4.13 (t, J=7.0 Hz, 2H), 3.43 (dd, J=13.0, 6.7 Hz, 2H), 1.94 (p, J=7.2 Hz, 2H), 1.65 (dt, J=14.9, 7.3 Hz, 2H), 1.30 (s, 9H). LC-MS m/z: 392.0 [M+Na]⁺. Purity (214 nm): 99%; t_{R}=8.89 min.

### Examples 188a and 188b—N-(4-(3-(tert-Butyl)-1H-pyrazol-1-yl)butyl)-2-methoxy-5-morpholino-1H-benzo[d]imidazole-1-carboxamide and N-(4-(3-(tert-butyl)-1H-pyrazol-1-yl) butyl)-2-methoxy-6-morpholino-1H-benzo[d]imidazole-1-carboxamide

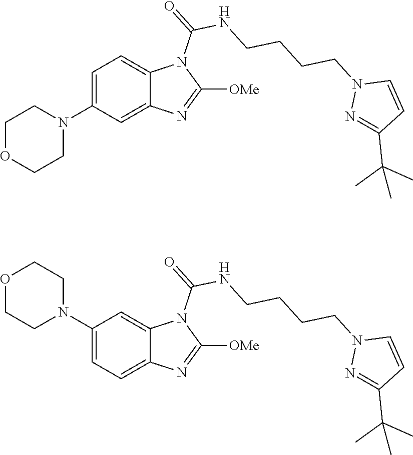 [SMILES: COc1nc2cc(N3CCOCC3)ccc2n1C(=O)NCCCCn1ccc(C(C)(C)C)n1 ; COc1nc2ccc(N3CCOCC3)cc2n1C(=O)NCCCCn1ccc(C(C)(C)C)n1]

Following general procedure C, 4-(2-methoxy-1H-benzo[d]imidazol-5-yl)morpholine (100 mg, 0.43 mmol) and 4-(3-(tert-butyl)-1H-pyrazol-1-yl)butan-1-amine (167 mg, 1.85 mmol) afforded N-(4-(3-(tert-butyl)-1H-pyrazol-1-yl)butyl)-2-methoxy-5-morpholino-1H-benzo[d]imidazole-1-carboxamide (64.4 mg, 27.6%) and N-(4-(3-(tert-butyl)-1H-pyrazol-1-yl)butyl)-2-methoxy-6-morpholino-1H-benzo[d]imidazole-1-carboxamide (44.8 mg, 19.2%) both as clear oils.

N-(4-(3-(tert-butyl)-1H-pyrazol-1-yl)butyl)-2-methoxy-5-morpholino-1H-benzo[d]imidazole-1-carboxamide: ¹H NMR (400 MHz, CDCl₃) δ 8.01 (d, J=9.2 Hz, 1H), 7.26 (s, 1H), 7.06 (d, J=2.4 Hz, 1H), 6.95 (t, J=5.6 Hz, 1H), 6.88 (dd, J=8.8 Hz, 2.4 Hz, 1H), 6.07 (d, J=2.4 Hz, 1H), 4.29 (s, 3H), 4.13 (t, J=7.2 Hz, 2H), 3.89-3.87 (m, 4H), 3.41 (q, J=6.8 Hz, 2H), 3.17-3.14 (m, 4H), 1.95-1.91 (m, 2H), 1.65-1.60 (m, 2H), 1.29 (s, 9H). LC-MS m/z: 477.1 [M+23]⁺. HPLC Purity (214 nm): 99%; t_{R}=7.28 min.

N-(4-(3-(tert-butyl)-1H-pyrazol-1-yl)butyl)-2-methoxy-6-morpholino-1H-benzo[d]imidazole-1-carboxamide: ¹H NMR (400 MHz, CDCl₃) δ 7.80 (d, J=2.4 Hz, 1H), 7.39 (d, J=8.8 Hz, 1H), 7.26 (s, 1H), 7.02 (t, J=5.6 Hz, 1H), 6.90 (dd, J=8.4 Hz, 2.4 Hz, 1H), 6.07 (d, J=2.4 Hz, 1H), 4.28 (s, 3H), 4.12 (t, J=7.6 Hz, 2H), 3.88-3.86 (m, 4H), 3.41 (q, J=6.8 Hz, 2H), 3.18-3.16 (m, 4H), 1.97-1.89 (m, 2H), 1.68-1.60 (m, 2H), 1.30 (s, 9H). LC-MS m/z: 477.1 [M+23]⁺. HPLC Purity (214 nm): 99%; t_{R}=7.27 min.

### Examples 189a and 189b —2-Ethoxy-5-morpholino-N-(3-phenylpropyl)-1H-benzo[d]imidazole-1-carboxamide and 2-ethoxy-6-morpholino-N-(3-phenylpropyl)-1H-benzo[d]imidazole-1-carboxamide

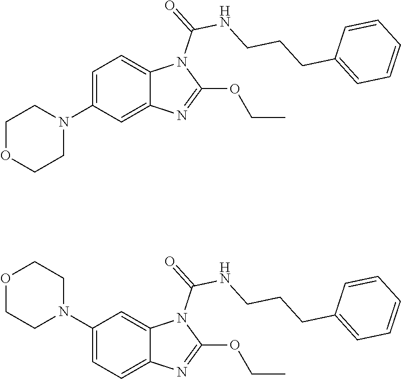 [SMILES: CCOc1nc2cc(N3CCOCC3)ccc2n1C(=O)NCCCc1ccccc1 ; CCOc1nc2ccc(N3CCOCC3)cc2n1C(=O)NCCCc1ccccc1]

Following general procedure E, 5-fluoro-2-nitrobenzenamine (6.0 g, 38.5 mmol) and morpholine (4.8 g, 46.2 mmol) afforded 5-morpholino-2-nitrobenzenamine (6.7 g, 78.0%) as a yellow solid. LC-MS m/z: 224.0 [M+H]⁺. Purity (254 nm): 100.0%; t_{R}=1.60 min.

Following general procedure F, 5-morpholino-2-nitrobenzenamine (6.7 g, 30.0 mmol) afforded 4-morpholinobenzene-1,2-diamine (5.0 g, 86.4%) as a black oil. LC-MS m/z: 194.2 [M+H]⁺. Purity (214 nm): 98.2%; t_{R}=0.80 min.

Following general procedure G (method A), 4-morpholinobenzene-1,2-diamine (5.0, 25.9 mmol) afforded 4-(2-ethoxy-3H-benzo[d]imidazol-5-yl)-morpholine (4.6 g, 71.4%) as a black oil. LC-MS m/z: 248.2 [M+H]⁺. Purity (214 nm): 98.9%; t_{R}=1.44 min.

Following general procedure C, 4-(2-ethoxy-3H-benzo[d]imidazol-5-yl)-morpholine (1.3 g, 5.3 mmol) and 3-phenylpropan-1-amine (864.5 mg, 6.4 mmol) afforded 2-ethoxy-5-morpholino-N-(3-phenylpropyl)-1H-benzo[d]imidazole-1-carboxamide (32.2 mg, 3.3%) and 2-ethoxy-6-morpholino-N-(3-phenylpropyl)-1H-benzo[d]imidazole-1-carboxamide (30.3 mg, 2.9%) both as white solids.

2-ethoxy-5-morpholino-N-(3-phenylpropyl)-1H-benzo[d]imidazole-1-carboxamide ¹H NMR (400 MHz, CDCl₃) δ 8.03 (d, J=8.9 Hz, 1H), 7.31-7.26 (m, 2H), 7.24-7.17 (m, 3H), 7.05 (d, J=2.3 Hz, 1H), 7.01 (bs, 1H), 6.88 (dd, J=8.9, 2.4 Hz, 1H), 4.73 (q, J=7.1 Hz, 2H), 3.98-3.89 (m, 4H), 3.47 (dd, J=12.8, 6.9 Hz, 2H), 3.22-3.15 (m, 4H), 2.73 (t, J=7.6 Hz, 2H), 1.98 (p, J=7.2 Hz, 2H), 1.55 (t, J=7.1 Hz, 3H). LC-MS m/z: 409.3 [M+H]⁺. HPLC Purity (214 nm): 100.0%; t_{R}=8.56 min.

2-ethoxy-6-morpholino-N-(3-phenylpropyl)-1H-benzo[d]imidazole-1-carboxamide ¹H NMR (400 MHz, CDCl₃) δ 7.83 (d, J=2.4 Hz, 1H), 7.38 (d, J=8.6 Hz, 1H), 7.32-7.28 (m, 2H), 7.22-7.18 (m, 3H), 7.09 (bs, 1H), 6.90 (dd, J=8.7, 2.5 Hz, 1H), 4.71 (q, J=7.1 Hz, 2H), 3.95-3.88 (m, 4H), 3.46 (dd, J=12.7, 6.9 Hz, 2H), 3.23-3.17 (m, 4H), 2.74 (t, J=7.5 Hz, 2H), 2.00 (p, J=7.2 Hz, 2H), 1.53 (t, J=7.1 Hz, 3H). LC-MS m/z: 409.3 [M+H]⁺. HPLC Purity (214 nm): 98.24%; t_{R}=7.73 min.

### Examples 190a and 190b—2-(2-Methoxyethoxy)-5-morpholino-N-(3-phenylpropyl)-1H-benzo[d]imidazole-1 carboxamide and 2-(2-methoxyethoxy)-6-morpholino-N-(3-phenyl propyl)-1H-benzo[d]imidazole-1-carboxamide

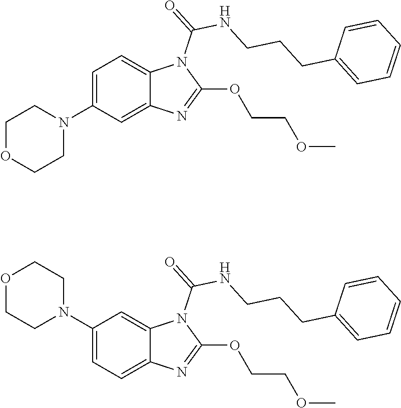 [SMILES: COCCOc1nc2cc(N3CCOCC3)ccc2n1C(=O)NCCCc1ccccc1 ; COCCOc1nc2ccc(N3CCOCC3)cc2n1C(=O)NCCCc1ccccc1]

Following general procedure H, 6-bromo-2-(2-methoxyethoxy)-1H-benzo[d]imidazole (2.00 g, 7.38 mmol) and morpholine (1.28 g, 14.75 mmol) afforded 2-(2-methoxyethoxy)-6-morpholino-1H-benzo[d]-imidazole (0.4 g, 19.9%) as a yellow solid. LC-MS m/z: 278.1 [M+H]⁺. HPLC Purity (254 nm): 96%; t_{R}=1.41 min.

Following general procedure C, 2-(2-methoxyethoxy)-6-morpholino-1H-benzo[d]imidazole (0.20 g, 0.72 mmol) and 3-phenylpropan-1-amine (0.1073 g, 0.79 mmol) afforded 2-(2-methoxyethoxy)-5-morpholino-N-(3-phenylpropyl)-1H-benzo[d]imidazole-1 carboxamide (17.1 mg, 5.4%) and 2-(2-methoxyethoxy)-6-morpholino-N-(3-phenylpropyl)-1H-benzo[d]imidazole-1-carboxamide (100.2 mg, 32.2%) as white solids.

2-(2-methoxyethoxy)-5-morpholino-N-(3-phenylpropyl)-1H-benzo[d]imidazole-1 carboxamide: ¹H NMR (400 MHz, CDCl₃) δ 7.82 (d, J=1.6 Hz, 1H), 7.37 (d, J=8.4 Hz, 1H), 7.31-7.27 (m, 3H), 7.26-7.19 (m, 3H), 6.91 (dd, J=8.8, 1.6 Hz, 1H), 4.76 (t, J=4.4 Hz, 2H), 3.87 (t, J=4.8 Hz, 4H), 3.79 (t, J=4.8 Hz, 2H), 3.46 (dd, J=6.0, 2.8 Hz, 2H), 3.46 (s, 3H), 3.17 (t, J=4.4 Hz, 4H), 2.74 (t, J=8.0 Hz, 2H), 1.97 (p, J=7.2 Hz, 2H). LC-MS m/z: 439.0 [M+H]⁺. HPLC Purity (214 nm): >99%; t_{R}=8.40 min.

2-(2-methoxyethoxy)-6-morpholino-N-(3-phenylpropyl)-1H-benzo[d]imidazole-1-carboxamide: ¹H NMR (400 MHz, CDCl₃) δ 8.03 (d, J=8.8 Hz, 1H), 7.30-7.26 (m, 2H), 7.22-7.17 (m, 4H), 7.04 (d, J=2.4 Hz, 1H), 6.88 (dd, J=8.8, 1.5 Hz, 1H), 4.79-4.77 (m, 2H), 3.87 (t, J=4.8 Hz, 4H), 3.82-3.79 (m, 2H), 3.46 (dd, J=5.2 Hz, 2.8 Hz, 2H), 3.48 (s, 3H), 3.14 (t, J=4.4 Hz, 4H), 2.74 (t, J=8.0 Hz, 2H), 1.97 (p, J=7.2 Hz, 2H). LC-MS m/z: 439.0 [M+H]⁺. HPLC Purity (214 nm): >99%; t_{R}=8.46 min.

### Example 191—2-(2-Methoxyethoxy)-N-(3-phenylpropyl)-1H-benzo[d]imidazole-1-carboxamide

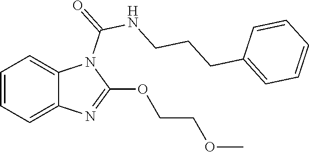 [SMILES: COCCOc1nc2ccccc2n1C(=O)NCCCc1ccccc1]

A mixture of NaH (8.45 g, 211 mmol) and 2-methoxyethanol (38.70 g, 509 mmol) at 0° C. was allowed to warm to RT and stirred for 30 min. Then 6-bromo-2-(methylsulfonyl)-1H-benzo[d]imidazole (7.00 g, 25.4 mmol) was added and the mixture was stirred at 110° C. for 48 hrs. The the mixture was cooled to RT and quenched with 2N HCl (100 mL), extracted with DCM (100 mL×3), dried over anhydrous Na₂SO₄, filtered and concentrated. The residue was purified by SGC (DCM/MeOH=10/1) to give 6-bromo-2-(2-methoxyethoxy)-1H-benzo[d]-imidazole (2.6 g, 37.7%) as a yellow solid. LC-MS m/z: 273.0 [M+H]⁺. HPLC Purity (214 nm): 100%; t_{R}=1.67 min.

Following general procedure F, 6-bromo-2-(2-methoxyethoxy)-1H-benzo[d]imidazole (0.50 g, 1.84 mmol) afforded 2-(2-methoxyethoxy)-1H-benzo[d]imidazole (0.3 g, 85.7%) as a yellow solid. LC-MS m/z: 193.2 [M+H]⁺. HPLC Purity (214 nm): 85%; t_{R}=1.44 min.

Following general procedure C, 2-(2-methoxyethoxy)-1H-benzo[d]imidazole (0.15 g, 0.78 mmol) and 3-phenylpropan-1-amine (0.12 g, 0.86 mmol) afforded the title compound (91.6 mg, 33.3%) as a white solid. ¹H NMR (400 MHz, CDCl₃) δ 8.17 (d, J=8.0 Hz, 1H), 7.49 (d, J=7.2 Hz, 1H), 7.31-7.19 (m, 8H), 4.80 (t, J=5.2 Hz, 2H), 3.81 (t, J=3.6 Hz, 2H), 3.46 (q, J=6.4 Hz, 2H), 3.39 (s, 3H), 2.74 (t, J=7.6 Hz, 2H), 1.97 (p, J=8.0 Hz, 2H). LC-MS m/z: 354.0 [M+H]⁺. HPLC Purity (214 nm): >99%; t_{R}=8.81 min.

### Example 192—4-(4-Methylpiperazin-1-yl)-N-(3-phenylpropyl)-1H-benzo[d]imidazole-1-carboxamide

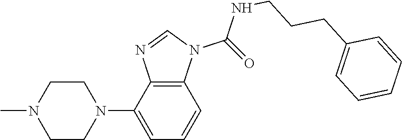 [SMILES: CN1CCN(c2cccc3c2ncn3C(=O)NCCCc2ccccc2)CC1]

Following general procedure E, 3-fluoro-2-nitroaniline (1.0 g, 6.4 mmol) and 1-methylpiperazine (645 mg, 6.4 mmol) afforded 3-(4-methylpiperazin-1-yl)-2-nitroaniline (1.41 g, 93.2%) as a yellow solid. LC-MS m/z: 236.27 [M+H]⁺. Purity (214 nm): 99.5%; t_{R}=1.57 min.

Following general procedure F, 3-(4-methylpiperazin-1-yl)-2-nitroaniline (1.4 g, 5.93 mmol) afforded 3-(4-methylpiperazin-1-yl)benzene-1,2-diamine (1.2 g, 98%) as a yellow solid. LC-MS m/z: 206.29 [M+H]⁺. Purity (214 nm): 94%; t_{R}=1.29 min.

Following general procedure G (method B), 3-(4-methylpiperazin-1-yl)benzene-1,2-diamine (600 mg, 3.66 mmol) afforded crude 4-(4-methylpiperazin-1-yl)-1H-benzo[d]imidazole (587 mg, 93.3%) as a black solid which was used directly in the next step. LC-MS m/z: 216.28 [M+H]⁺. Purity (214 nm): 99.21%; t_{R}=1.17 min.

Following general procedure C, 4-(4-methylpiperazin-1-yl)-1H-benzo[d]imidazole (587 mg, 2.71 mmol) and 3-phenylpropan-1-amine (550 mg, 4.07 mmol) afforded 4-(4-methylpiperazin-1-yl)-N-(3-phenylpropyl)-1H-benzo[d]imidazole-1-carboxamide (137 mg, 12.7%) as a white solid, which was triturated with MeCN (3 mL). The filter cake was collected to give the title compound the title compound (29.5 mg, 3.4%) as a white solid. 1H NMR (400 MHz, CDCl₃) δ 8.20 (s, 1H), 7.34-7.27 (m, 2H), 7.25-7.18 (m, 5H), 6.74 (dd, J=7.6 Hz, 1H), 5.69 (t, 1H), 3.62 (bs, 4H), 3.55 (q, J=6.8 Hz, 2H), 2.78 (t, J=7.4 Hz, 4H), 2.78 (t, J=6.4 Hz, 2H), 2.49 (s, 3H), 2.06 (p, J=6.0 Hz, 2H). LC-MS m/z: 378.1 [M+H]⁺. HPLC Purity (214 nm): 98.38%; t_{R}=5.46 min.

### Example 193—N-iso-Butyl-2-methoxy-5-(1-methylpiperidin-4-yl)-1H-benzo[d]imidazole-1-carboxamide

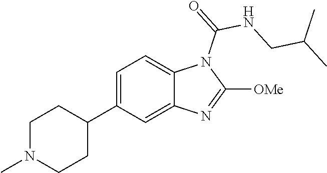 [SMILES: COc1nc2cc(C3CCN(C)CC3)ccc2n1C(=O)NCC(C)C]

Following general procedure A, 6-bromo-2-methoxy-1H-benzo[d]imidazole (678 mg, 3.0 mmol) and 1-methyl-4-(4,4,5,5-tetramethyl-1,3,2-dioxaborolan-2-yl)-1,2,3,6-tetrahydropyridine (869 mg, 3.9 mmol) afforded 2-methoxy-6-(1-methyl-1,2,3,6-tetrahydropyridin-4-yl)-1H-benzo[d]imidazole (590 mg, 81%) as a yellow oil. LC-MS m/z: 244.1 [M+H]⁺. Purity (214 nm): 87%; t_{R}=0.86 min.

To a solution of 2-methoxy-6-(1-methyl-1,2,3,6-tetrahydropyridin-4-yl)-1H-benzo[d]imidazole (243 mg, 1.0 mmol) in MeOH (8 mL) was added Pd/C (100 mg, 10%). The mixture was stirred at RT under H2 for 16 h. The mixture was filtered and concentrated to give 2-methoxy-6-(1-methylpiperidin-4-yl)-1H-benzo[d]imidazole (245 mg, 100%) as a yellow oil. LC-MS m/z: 246.2 [M+H]⁺. Purity (214 nm): 82%; t_{R}=1.19 min.

Following general procedure C, 2-methoxy-6-(1-methylpiperidin-4-yl)-1H-benzo[d]imidazole (98 mg, 0.4 mmol) and iso-butylamine (29.2 mg, 0.4 mmol) afforded the title compound (27.4 mg, 20%) as a white solid. ¹H NMR (400 MHz, CDCl₃) δ 8.05 (s, 1H), 7.48 (d, J=8.0 Hz, 1H), 7.22 (dd, J=8.0, 1.2 Hz, 1H), 7.03 (bs, 1H), 4.33 (s, 3H), 3.57-3.51 (m, 2H), 3.27 (t, J=6.0 Hz, 2H), 2.84-2.81 (m, 6H), 2.53-2.50 (m, 2H), 2.05 (d, J=13.6 Hz, 2H), 1.93 (sept, J=6.8 Hz, 1H), 1.00 (d, J=6.8 Hz, 6H). LC-MS m/z: 345.1 [M+H]⁺. HPLC Purity (214 nm): 95.96%; t_{R}=5.76 min.

### Example 194—2-Methoxy-5-(4-methylpiperazin-1-yl)-N-(3-phenylpropyl)-1H-benzo[d]imidazole-1-carboxamide

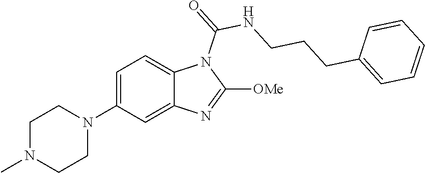 [SMILES: COc1nc2cc(N3CCN(C)CC3)ccc2n1C(=O)NCCCc1ccccc1]

Following general procedure E, 5-fluoro-2-nitroaniline (2.00 g, 12.82 mmol) and 1-methylpiperazine (2.57 g, 25.64 mmol) afforded 5-(4-methylpiperazin-1-yl)-2-nitroaniline (2.0 g, 66.7%) as a yellow solid. LC-MS m/z: 237.2 [M+H]⁺. HPLC Purity (214 nm): 97%; t_{R}=1.56 min.

Following general procedure F, 5-(4-methylpiperazin-1-yl)-2-nitroaniline (2.00 g, 8.47 mmol) afforded 4-(4-methylpiperazin-1-yl)benzene-1,2-diamine (2.0 g) as a yellow solid. LC-MS m/z: 205.2 [M+H]⁺. HPLC Purity (214 nm): 96%; t_{R}=0.59 min.

Following general procedure G (method A), 4-(4-methylpiperazin-1-yl)benzene-1,2-diamine (1.50 g, 7.28 mmol) afforded 2-methoxy-6-(4-methylpiperazin-1-yl)-1H-benzo[d]imidazole (1.5 g, 83.3%) as a yellow solid. LC-MS m/z: 247.2 [M+H]⁺. HPLC Purity (254 nm): 99%; t_{R}=1.57 min.

Following general procedure C, 2-methoxy-6-(4-methylpiperazin-1-yl)-1H-benzo[d]imidazole (0.50 g, 2.03 mmol) and 3-phenylpropan-1-amine (0.30 g, 2.23 mmol) afforded the title compound (60.9 mg, 7.2%) as a white solid. ¹H NMR (400 MHz, CDCl₃) δ 7.84 (d, J=6.8, 2.0 Hz, 1H), 7.39 (d, J=8.4 Hz, 1H), 7.31-7.28 (m, 2H), 7.27-7.20 (m, 3H), 6.96 (bs, 1H), 6.90 (dd, J=8.4, 2.4 Hz, 1H), 4.27 (s, 3H), 3.45 (dd, J=13.2 Hz, 6.8 Hz, 2H), 3.31 (t, J=5.2 Hz, 4H), 2.89 (t, J=4.8 Hz, 4H), 2.73 (t, J=7.6 Hz, 2H), 2.52 (s, 3H), 1.99 (p, J=7.6 Hz, 2H). LC-MS m/z: 408.1 [M+H]⁺. HPLC Purity (214 nm): >96%; t_{R}=6.94 min.

### Examples 195a and 195b —2-Methoxy-5-(1-methylpiperidin-4-yl)-N-(3-phenylpropyl)-1H-benzo[d]imidazole-1-carboxamide and 2-methoxy-6-(1-methylpiperidin-4-yl)-N-(3-phenylpropyl)-1H-benzo[d]imidazole-1-carboxamide

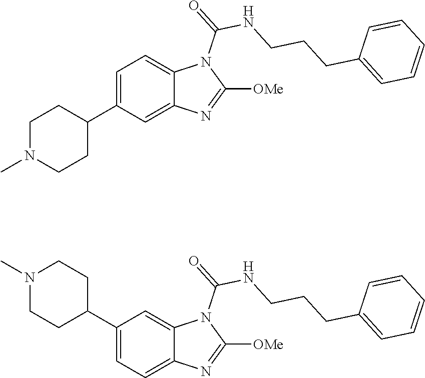 [SMILES: COc1nc2cc(C3CCN(C)CC3)ccc2n1C(=O)NCCCc1ccccc1 ; COc1nc2ccc(C3CCN(C)CC3)cc2n1C(=O)NCCCc1ccccc1]

Following general procedure G (method A), 4-bromobenzene-1,2-diamine (5.0 g, 26.77 mmol) afforded 6-bromo-2-methoxy-1H-benzo[d]imidazole (3.8 g, 62.6%) as a brown-red solid. LC-MS m/z: 227.0 [M+H]⁺. Purity (254 nm): 85%; t_{R}=1.65 min.

Following general procedure A, 6-bromo-2-methoxy-1H-benzo[d]imidazole (2.0 g, 8.81 mmol) and 1-methyl-4-(4,4,5,5-tetramethyl-1,3,2-dioxaborolan-2-yl)-1,2,3,6-tetrahydropyridine (3.93 g, 17.62 mmol) afforded 2-methoxy-6-(1-methyl-1,2,3,6-tetrahydropyridin-4-yl)-1H-benzo[d]imidazole (1.61 g, 75.2%) as a yellow solid. LC-MS m/z: 244.2. [M+H]⁺. Purity (214 nm): 85%; t_{R}=1.41 min.

To a solution of 2-methoxy-6-(1-methyl-1,2,3,6-tetrahydropyridin-4-yl)-1H-benzo[d]-imidazole (1.60 g, 6.58 mmol) in EtOH (20 mL) was added Pd/C (800 mg) and the reaction mixture was stirred at 50° C. under H₂ for 2 h. The reaction was filtered and concentrated to afford 2-methoxy-6-(1-methylpiperidin-4-yl)-1H-benzo[d]imidazole as a white solid (1.26 g, 77.9%). LC-MS m/z: 246.2 [M+H]⁺. Purity (214 nm): 70%; t_{R}=1.32 min.

Following general procedure C, 2-methoxy-6-(1-methylpiperidin-4-yl)-1H-benzo[d]imidazole (300 mg, 1.22 mmol) and 3-phenylpropan-1-amine (248 mg, 1.83 mmol) afforded 2-methoxy-5-(1-methylpiperidin-4-yl)-N-(3-phenylpropyl)-1H-benzo[d]imidazole-1-carboxamide (12.3 mg, 2.5%) and 2-methoxy-6-(1-methylpiperidin-4-yl)-N-(3-phenylpropyl)-1H-benzo[d]imidazole-1-carboxamide (18.1 mg, 3.6%) as white solids.

2-methoxy-5-(1-methylpiperidin-4-yl)-N-(3-phenylpropyl)-1H-benzo[d]imidazole-1-carboxamide ¹H NMR (400 MHz, CDCl₃) δ 8.58 (bs, 1H), 8.06 (s, 1H), 7.45 (d, J=8.2 Hz, 1H), 7.35-7.27 (m, 2H), 7.20 (s, 2H), 7.16 (d, J=8.1 Hz, 1H), 6.97 (bs, 1H), 4.28 (ss, 3H), 3.58-3.39 (m, 4H), 2.80-2.61 (m, 7H), 2.24-2.11 (m, 2H), 2.09-1.92 (m, 4H). LC-MS m/z: 407.1 [M+H]⁺. HPLC Purity (254 nm): 100%; t_{R}=5.72 min.

2-methoxy-6-(1-methylpiperidin-4-yl)-N-(3-phenylpropyl)-1H-benzo[d]imidazole-1-carboxamide ¹H NMR (400 MHz, CDCl₃) δ 8.08 (d, J=8.3 Hz, 1H), 7.38 (s, 1H), 7.29-7.18 (m, 4H), 7.09 (d, J=7.9 Hz, 1H), 6.91 (bs, 1H), 4.29 (s, 3H), 3.46 (bs 4H), 2.73 (t, J=7.4 Hz, 3H), 2.65 (bs, 4H), 2.17 (bs, 2H), 2.04-1.96 (m, 4H). LC-MS m/z: 407.1 [M+H]⁺. HPLC Purity (254 nm): 100%; t_{R}=5.72 min.

### Example 196—2-Methoxy-6-morpholino-N-(3-(thiazol-2-yl)propyl)-1H-benzo[d]imidazole-1-carboxamide

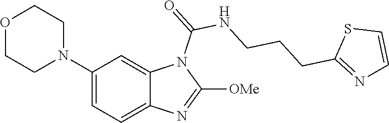 [SMILES: COc1nc2ccc(N3CCOCC3)cc2n1C(=O)NCCCc1nccs1]

Following general procedure C, 4-(2-methoxy-1H-benzo[d]imidazol-6-yl)morpholine (0.25 g, 1.07 mmol) and 3-(thiazol-2-yl)propan-1-amine (0.17 g, 1.18 mmol) afforded the title compound (12.5 mg, 3.6%) as a white solid. ¹H NMR (400 MHz, CDCl₃) δ 8.02 (d, J=9.2 Hz, 1H), 7.68 (d, J=3.2 Hz, 1H), 7.20 (d, J=3.6 Hz, 1H), 7.16 (bs, 1H), 7.06 (d, J=2.4 Hz, 1H), 6.88 (dd, J=8.8, 2.4 Hz, 1H), 4.30 (s, 3H), 3.89 (t, J=4.4 Hz, 4H), 3.53 (q, J=6.0 Hz, 2H), 3.18-3.12 (m, 6H), 2.17 (p, J=7.2 Hz, 2H). LC-MS m/z: 402.5 [M+H]⁺. HPLC Purity (214 nm): >99%; t_{R}=6.44 min.

### Example 197—4-(3,4-Dimethylpiperazin-1-yl)-N-iso-pentyl-1H-benzo[d]imidazole-1-carboxamide

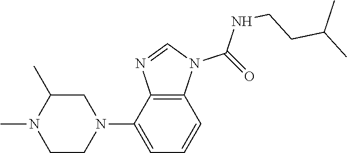 [SMILES: CC(C)CCNC(=O)n1cnc2c(N3CCN(C)C(C)C3)cccc21]

Following general procedure E, 3-fluoro-2-nitroaniline (102.3 mg, 0.66 mmol) and 1,2-dimethylpiperazine (75 mg, 0.66 mmol) afforded crude 3-(3,4-dimethylpiperazin-1-yl)-2-nitroaniline (180 mg, 95%) as an off yellow oil. LC-MS m/z: 251.4 [M+H]⁺. Purity (214 nm): 95%; t_{R}=1.64 min.

Following general procedure F, 3-(3,4-dimethylpiperazin-1-yl)-2-nitroaniline (180 mg, 0.72 mmol) afforded crude 3-(3,4-dimethylpiperazin-1-yl)benzene-1,2-diamine (150 mg, 93%) as a yellow oil. LC-MS m/z: 221.4 [M+H]⁺. Purity (214 nm): 93%; t_{R}=1.28 min.

Following general procedure G (method B), 3-(3,4-dimethylpiperazin-1-yl)benzene-1,2-diamine (150 mg, 0.68 mmol) afforded crude 4-(3,4-dimethylpiperazin-1-yl)-1H-benzo[d]imidazole (180 mg, 90%) as a yellow oil. LC-MS m/z: 231.3 [M+H]⁺. Purity (214 nm): 90%; t_{R}=1.26 min.

Following general procedure C, 4-(3,4-dimethylpiperazin-1-yl)-1H-benzo[d]imidazole (180 mg, 0.78 mmol) and 3-methylbutan-1-amine (67.9 mg, 0.78 mmol) afforded the title compound (88.4 mg, 8.8%) as a yellow oil. ¹H NMR (400 MHz, CDCl₃) δ 8.47 (s, 1H), 8.44 (s, 1H), 7.42 (d, J=8.1 Hz, 1H), 6.72 (d, J=8.0 Hz, 1H), 6.35 (bs, 1H), 4.15-3.99 (m, 2H), 3.52 (dd, J=13.9, 6.6 Hz, 2H), 3.47-3.29 (m, 2H), 3.14-2.94 (m, 3H), 2.65 (s, 3H), 1.72 (dd, J=13.2, 6.6 Hz, 1H), 1.59 (dd, J=14.6, 7.1 Hz, 2H), 1.38 (d, J=6.0 Hz, 3H), 0.99 (d, J=6.5 Hz, 6H). LC-MS m/z: 344.2 [M+H]⁺. HPLC: Purity (214 nm): 100%; t_{R}=7.81 min.

### Example 198—4-(Hexahydro-1H-pyrido[1,2-a]pyrazin-2(6H)-yl)-N-iso-pentyl-1H-benzo[d]imidazole-1-carboxamide

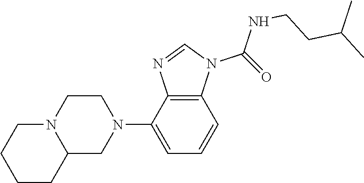 [SMILES: CC(C)CCNC(=O)n1cnc2c(N3CCN4CCCCC4C3)cccc21]

Following general procedure E, 3-fluoro-2-nitrobenzenamine (300.0 mg, 1.9 mmol) and octahydro-1H-pyrido[1,2-a]pyrazine (323.1 mg, 2.3 mmol) afforded 3-(hexahydro-1H-pyrido[1,2-a]-pyrazin-2(6H)-yl)-2-nitrobenzenamine (400.0 mg, 76.3%) as a yellow solid. LC-MS m/z: 277.2 [M+H]⁺. Purity (254 nm): 100.0%; t_{R}=1.77 min.

Following general procedure F, 3-(hexahydro-1H-pyrido[1,2-a]pyrazin-2(6H)-yl)-2-nitrobenzenamine (400.0 mg, 1.4 mmol) afforded crude 3-(hexahydro-1H-pyrido[1,2-a]-pyrazin-2(6H)-yl)benzene-1,2-diamine (300.0 mg, 84.7%) as a black oil which was used directly in the next step. LC-MS m/z: 247.2 [M+H]⁺. Purity (214 nm): 92.6%; t_{R}=1.51 min.

Following general procedure G (method B), 3-(hexahydro-1H-pyrido[1,2-a]pyrazin-2(6H)-yl)benzene-1,2-diamine (300.0 mg, 1.2 mmol) afforded crude 2-(1H-benzo[d]imidazol-4-yl)-octahydro-1H-pyrido[1,2-a]pyrazine (240.0 mg, 78.1%) as a black oil which was used directly in the next step. LC-MS m/z: 257.2 [M+H]⁺. Purity (254 nm): 100.0%; t_{R}=1.49 min.

Following general procedure B, 2-(1H-benzo[d]imidazol-4-yl)-octahydro-1H-pyrido[1,2-a]pyrazine (200 mg, 0.8 mmol) and 3-methylbutan-1-amine (95.7 mg, 1.1 mmol) afforded the title compound (220.4 mg, 46.7%) as a white solid. ¹H NMR (400 MHz, CDCl₃) δ 8.49 (s, 1H), 7.47 (d, J=8.2 Hz, 1H), 7.29-7.22 (m, 1H), 6.71 (d, J=8.0 Hz, 2H), 4.17 (d, J=12.5 Hz, 1H), 4.02 (d, J=12.1 Hz, 1H), 3.51 (dd, J=14.5, 6.1 Hz, 2H), 3.47-3.34 (m, 2H), 3.29 (t, J=11.5 Hz, 1H), 3.04 (t, J=11.4 Hz, 1H), 2.92 (dd, J=13.4, 10.4 Hz, 2H), 2.44 (t, J=11.8 Hz, 1H), 1.95 (dd, J=28.7, 12.9 Hz, 2H), 1.84-1.65 (m, 4H), 1.58 (dt, J=16.2, 8.3 Hz, 2H), 1.50-1.40 (m, 1H), 0.98 (d, J=6.6 Hz, 6H). LC-MS m/z: 370.3 [M+H]⁺. HPLC Purity (214 nm): 98.52%; t_{R}=5.24 min.

### Example 199—(R)-4-(Hexahydropyrazino[2,1-c][1,4]oxazin-8(1H)-yl)-N-isopentyl-1H-benzo[d]imidazole-1-carboxamide

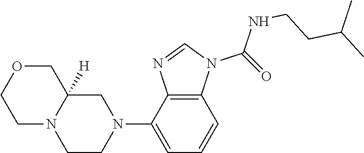 [SMILES: CC(C)CCNC(=O)n1cnc2c(N3CCN4CCOC[C@H]4C3)cccc21]

Following general procedure E, 3-fluoro-2-nitroaniline (250 mg, 1.6 mmol) and (R)-octahydropyrazino-[2,1-c][1,4]oxazine (300 mg, 1.6 mmol) afforded 3-(hexahydropyrazino[2,1-c][1,4]oxazin-8(1H)-yl)-2-nitroaniline (100 mg, 22%). LC-MS m/z: 279.2[M+H]⁺. Purity (214 nm): 80%; t_{R}=1.59 min.

Following general procedure F, 3-(hexahydropyrazino[2,1-c][1,4]oxazin-8(1H)-yl)-2-nitroaniline (100 mg, 3.2 mmol) afforded 3-(hexahydropyrazino[2,1-c][1,4]oxazin-8(1H)-yl)benzene-1,2-diamine (90 mg, 90.0%). LC-MS m/z: 249.2 [M+H]⁺. Purity (214 nm): 90%; t_{R}=0.2 min.

Following general procedure G (method B), 3-(hexahydropyrazino[2,1-c][1,4]oxazin-8(1H)-yl)benzene-1,2-diamine (90 mg, 0.36 mmol) afforded 8-(1H-benzo[d]imidazol-4-yl)octahydropyrazino[2,1-c][1,4]oxazine (40 mg, 42.7%). LC-MS m/z: 259.2 [M+H]⁺. Purity (214 nm): 70%; t_{R}=1.45 min.

Following general procedure C, 8-(1H-benzo[d]imidazol-4-yl)octahydropyrazino[2,1-c][1,4]oxazine (40 mg, 0.15 mmol) and methylbutan-1-amine (13 mg, 0.15 mmol) afforded the title compound (8.0 mg, 14.5%) as a red solid. ¹H NMR (400 MHz, CDCl₃) δ 8.35 (s, 1H), 7.31 (dd, J=8.0, 6.7 Hz, 2H), 6.73 (dd, J=7.0, 1.8 Hz, 1H), 5.73 (bs, 1H), 4.16 (d, J=11.7 Hz, 1H), 3.99 (d, J=11.0 Hz, 1H), 3.92 (d, J=8.1 Hz, 1H), 3.87-3.76 (m, 2H), 3.54 (dd, J=14.8, 5.8 Hz, 2H), 3.42 (t, J=10.7 Hz, 1H), 3.11 (d, J=10.6 Hz, 1H), 2.99 (d, J=10.7 Hz, 1H), 2.83 (d, J=11.4 Hz, 1H), 2.75 (t, J=10.2 Hz, 2H), 2.70-2.60 (m, 1H), 2.54 (dd, J=11.4, 8.4 Hz, 1H), 1.73 (dq, J=13.4, 6.6 Hz, 1H), 1.60 (dd, J=14.7, 7.1 Hz, 2H), 0.99 (d, J=6.6 Hz, 6H). LC-MS m/z: 372.0[M+H]⁺. HPLC Purity (214 nm): >96%; t_{R}=7.50 min.

### Example 200—4-(4-(2-Cyanoethyl)piperazin-1-yl)-N-iso-pentyl-1H-benzo[d]imidazole-1-carboxamide

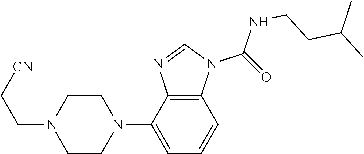 [SMILES: CC(C)CCNC(=O)n1cnc2c(N3CCN(CCC#N)CC3)cccc21]

Following general procedure E, 3-fluoro-2-nitroaniline (1.0 g, 6.8 mmol) and 3-(piperazin-1-yl)propanenitrile (800 g, 5.6 mmol) afforded 3-(4-(2-isocyanoethyl)-piperazin-1-yl)-2-nitroaniline (1.5 g, 97.4%) as a red solid. LC-MS m/z: 276.2 [M+H]⁺. Purity (214 nm): 80.72%; t_{R}=1.51 min.

Following general procedure F, 3-(4-(2-isocyanoethyl)piperazin-1-yl)-2-nitroaniline (750 mg, 2.7 mmol) afforded 3-(4-(2-isocyanoethyl)piperazin-1-yl)-benzene-1,2-diamine (659 mg, 85.8%) as a yellow oil. LC-MS m/z: 246.2 [M+H]⁺. Purity (214 nm): 87.67%; t_{R}=1.31 min.

Following general procedure G (method B), 3-(4-(2-isocyanoethyl)piperazin-1-yl)benzene-1,2-diamine (659 mg, 2.7 mmol) afforded 4-(4-(2-isocyanoethyl)piperazin-1-yl)-1H-benzo[d]imidazole (452 mg, 65.9%) as a red oil. LC-MS m/z: 256.2 [M+H]⁺. Purity (214 nm): 100%; t_{R}=1.31 min.

Following general procedure C, 4-(4-(2-isocyanoethyl)piperazin-1-yl)-1H-benzo[d]imidazole (452 mg, 1.77 mmol) and iso-butylamine (154 mg, 1.77 mmol) afforded the title compound (76.6 mg, 26.6%) as a white solid. ¹H NMR (400 MHz, CDCl₃) δ 8.40 (s, 1H), 7.40 (d, J=8.2 Hz, 1H), 7.35-7.24 (m, 1H), 6.74 (d, J=7.9 Hz, 1H), 6.11 (t, J=5.6 Hz, 1H), 3.61-3.49 (m, 6H), 2.90-2.81 (m, 6H), 2.61 (t, J=7.0 Hz, 2H), 1.71 (dp, J=6.6 Hz, 1H), 1.58 (dd, J=14.7, 7.1 Hz, 2H), 0.97 (d, J=6.6 Hz, 6H). LC-MS m/z: 369.0 [M+H]⁺. HPLC Purity (214 nm): 100%; t_{R}=6.67 min.

### Example 201—4-(3-(Fluoromethyl)-4-methylpiperazin-1-yl)-N-isopentyl-1H-benzo[d]imidazole-1-carboxamide

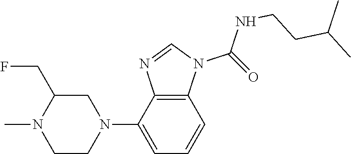 [SMILES: CC(C)CCNC(=O)n1cnc2c(N3CCN(C)C(CF)C3)cccc21]

Following general procedure E, 3-fluoro-2-nitroaniline (374 mg, 2.4 mmol) and 2-(fluoromethyl)piperazine (275 mg, 2.4 mmol) afforded 3-(3-(fluoromethyl)piperazin-1-yl)-2-nitroaniline (196 mg, 33.1%) as a yellow oil. LC-MS m/z: 255.2 [M+H]⁺. Purity (214 nm): 73.12%; t_{R}=0.33 min.

Following general procedure F, 3-(3-(fluoromethyl)piperazin-1-yl)-2-nitroaniline (196 mg, 0.77 mmol) afforded crude 3-(3-(fluoromethyl)piperazin-1-yl)benzene-1,2-diamine (202 mg, 95.3%) as a yellow oil. LC-MS m/z: 225.3 [M+H]⁺. Purity (214 nm): >70%; t_{R}=0.31 min.

Following general procedure G (method B), 3-(3-(fluoromethyl)piperazin-1-yl)benzene-1,2-diamine (202 mg, 0.9 mmol) afforded crude 4-(3-(fluoromethyl)piperazin-1-yl)-1H-benzo[d]imidazole (128 mg, 60.7%) as a yellow oil. LC-MS m/z: 235.31 [M+H]⁺. Purity (254 nm): 100%; t_{R}=1.26 min.

To a solution of 4-(3-(fluoromethyl)piperazin-1-yl)-1H-benzo[d]imidazole (128 mg, 0.55 mmol) in MeOH (10 mL) was added 37% CH₂O (62 mg, 0.76 mmol). The mixture was stirred at RT for 30 min and then NaBH₃CN (104 mg, 1.65 mmol) was added. The resulting mixture was stirred at RT overnight and then concentrated to afford a residue which was purified by silica gel column chromatography (DCM:MeOH=10:1) to give 4-(3-(fluoromethyl)-4-methylpiperazin-1-yl)-1H-benzo[d]imidazole (136 mg, 100%) as a yellow oil. LC-MS m/z: 249.2 [M+H]⁺. Purity (254 nm): 100%; t_{R}=0.97 min.

Following general procedure C, 4-(3-(fluoromethyl)-4-methylpiperazin-1-yl)-1H-benzo[d]imidazole (136 mg, 0.54 mmol) and iso-butylamine (48 mg, 0.54 mmol) afforded the title compound (14.0 mg, 7.2%) as a white solid. ¹H NMR (400 MHz, CDCl₃) δ 8.34 (s, 1H), 7.32-7.27 (m, 2H), 6.79-6.75 (m, 1H), 5.75 (bs, 1H), 4.67 (d, J=3.5 Hz, 1H), 4.55 (d, J=3.9 Hz, 1H), 4.07 (dd, J=28.3, 11.6 Hz, 2H), 3.54 (dd, J=14.2, 6.2 Hz, 2H), 3.09 (dt, J=11.3, 2.8 Hz, 1H), 3.01-2.84 (m, 2H), 2.80-2.65 (m, 2H), 2.47 (s, 3H), 1.84-1.67 (m, 1H), 1.62-1.49 (m, 2H), 0.99 (d, J=6.6 Hz, 6H). LC-MS m/z: 362.1 [M+H]⁺. HPLC Purity (214 nm): 100%; t_{R}=7.07 min. m/z

### Example 202—N-iso-Pentyl-4-(4-propylpiperazin-1-yl)-1H-benzo[d]imidazole-1-carboxamide

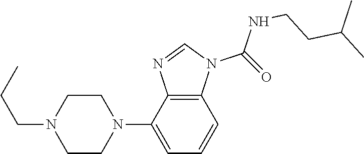 [SMILES: CCCN1CCN(c2cccc3c2ncn3C(=O)NCCC(C)C)CC1]

Following general procedure E, 3-fluoro-2-nitroaniline (5.0 g, 32 mmol) and tert-butyl piperazine-1-carboxylate (5.0 g, 26.7 mmol) afforded tert-butyl 4-(3-amino-2-nitrophenyl)-piperazine-1-carboxylate (7 g, 80.9%) as a yellow oil. LC-MS m/z: 323.1 [M+H]⁺. Purity (214 nm): 86.4%; t_{R}=1.38 min.

Following general procedure F, tert-butyl 4-(3-amino-2-nitrophenyl)piperazine-1-carboxylate (7 g, 22 mmol) afforded tert-butyl 4-(2,3-diaminophenyl)piperazine-1-carboxylate (5.4 g, 85.3%) as a yellow oil. LC-MS m/z: 293.2 [M+H]⁺. Purity (214 nm): 93.8%; t_{R}=1.1 min.

Following general procedure G (method B), tert-butyl 4-(2,3-diaminophenyl) piperazine-1-carboxylate (5.4 g, 18.5 mmol) afforded 4-(piperazin-1-yl)-1H-benzo[d]imidazole (2.3 g, 61.5%) as a red oil. LC-MS m/z: 203.1 [M+H]⁺. Purity (214 nm): 95.03%; t_{R}=0.81 min.

To a solution of 4-(piperazin-1-yl)-1H-benzo[d]imidazole (400 mg, 1.98 mmol) in MeOH (10 mL) was added propanal (161 mg, 2.77 mmol). The mixture was stirred at RT for 30 min and then NaBH₃CN (373 mg, 6 mmol) was added and the resulting mixture was stirred at RT overnight. The mixture was concentrated and purified by silica gel column chromatography (DCM:MeOH=10:1) to give 4-(4-propylpiperazin-1-yl)-1H-benzo[d]imidazole (467 mg, 96.7%) as a yellow oil. LC-MS m/z: 243.2 [M+H]⁺. Purity (214 nm): 77.58%; t_{R}=1.38 min.

Following general procedure C, 4-(4-propylpiperazin-1-yl)-1H-benzo[d]imidazole (467 mg, 1.91 mmol) and iso-butylamine (166 mg, 1.91 mmol) afforded the title compound (57.1 mg, 8.3%) as a white solid. ¹H NMR (400 MHz, CDCl₃) δ 8.35 (s, 1H), 7.36-7.32 (m, 2H), 6.76 (d, J=8.5 Hz, 1H), 5.76 (t, J=5.6 Hz, 1H), 3.70 (bs, 4H), 3.54 (dd, J=14.7, 5.9 Hz, 2H), 3.05 (bs, 4H), 2.88-2.50 (m, 2H), 1.74 (bs, 3H), 1.61 (q, J=6.8 Hz, 2H), 1.05-1.00 (m, 3H), 1.00 (d, J=7.6 Hz, 6H). LC-MS m/z: 358.1 [M+H]⁺. HPLC Purity (214 nm): 100%; t_{R}=6.84 min.

### Example 203—4-(4-Acetylpiperazin-1-yl)-N-isopentyl-1H-benzo[d]imidazole-1-carboxamide

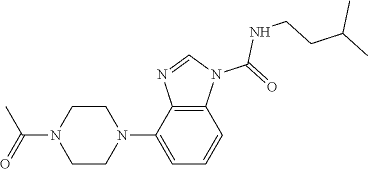 [SMILES: CC(=O)N1CCN(c2cccc3c2ncn3C(=O)NCCC(C)C)CC1]

Following general procedure E, 3-fluoro-2-nitroaniline (468 mg, 3.0 mmol) and 1-(piperazin-1-yl)ethan-1-one (384 mg, 3 mmol) after silica gel column chromatography (DCM:MeOH=20:1) afforded 1-(4-(3-amino-2-nitrophenyl)piperazin-1-yl)ethan-1-one (450 mg) as a yellow solid. LC-MS m/z: 265.2 [M+H]⁺. Purity (214 nm): 93.4%; t_{R}=1.44 min.

Following general procedure E, 1-(4-(3-amino-2-nitrophenyl)piperazin-1-yl)ethan-1-one (264 mg, 1.0 mmol) afforded crude 1-(4-(2,3-diaminophenyl)piperazin-1-yl)ethan-1-one (262 mg) as a yellow oil which was used directly in the next step. LC-MS m/z: 235.2 [M+H]⁺. Purity (214 nm): 90.4%; t_{R}=1.32 min.

Following general procedure G (method B), 1-(4-(2,3-diaminophenyl)piperazin-1-yl)ethan-1-one (234 mg, 1.0 mmol) afforded 1-(4-(1H-benzo[d]imidazol-4-yl)piperazin-1-yl)ethan-1-one (200 mg) as a yellow oil. LC-MS m/z: 245.2 [M+H]⁺. Purity (214 nm): 76.4%; t_{R}=1.30 min.

Following general procedure C, 1-(4-(1H-benzo[d]imidazol-4-yl)-piperazin-1-yl)ethan-1-one (114 mg, 0.5 mmol) and 3-methylbutan-1-amine (51.0 mg, 0.5 mmol) afforded the title compound (122.3 mg, 68.9%) as a white solid. ¹H NMR (400 MHz, CDCl₃) δ 8.37 (s, 1H), 7.39 (d, J=8.1 Hz, 1H), 7.30 (d, J=8.0 Hz, 1H), 6.72 (d, J=7.8 Hz, 1H), 6.08 (bs, 1H), 3.87 (t, J=4.8 Hz, 2H), 3.74 (t, J=4.8 Hz, 2H), 3.60-3.53 (m, 4H), 3.45 (t, J=4.0 Hz, 2H), 2.15 (s, 3H), 1.75 (sept, J=6.8 Hz, 1H), 1.63-1.47 (m, 2H), 0.99 (d, J=6.6 Hz, 6H). LC-MS m/z: 358.1 [M+H]⁺. HPLC Purity (214 nm): 100%; t_{R}=7.85 min.

### Example 204—N-iso-Pentyl-4-(4-(1-methylazetidin-3-yl)-piperazin-1-yl)-1H-benzo[d]imidazole-1-carboxamide

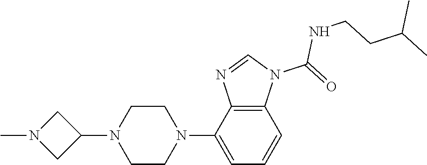 [SMILES: CC(C)CCNC(=O)n1cnc2c(N3CCN(C4CN(C)C4)CC3)cccc21]

To a solution of 4-(piperazin-1-yl)-1H-benzo[d]imidazole (500 mg, 2.5 mmol) in MeOH (15 mL) was added tert-butyl 3-oxoazetidine-1-carboxylate (513 mg, 3.0 mmol) and the mixture was stirred for 2 h. Then NaBH₃CN (465 mg, 7.5 mmol) was added at 0° C. and the solution was stirred at for 16 h. The reaction was quenched with water (10 ml), extracted with EA (50 mL), concentrated and purified by silica gel column chromatography (DCM/MeOH=20/1) to give tert-butyl 3-(4-(1H-benzo[d]imidazol-4-yl)piperazin-1-yl)azetidine-1-carboxylate (250 mg, 28.3%) as a yellow oil. LC-MS m/z: 358.1 [M+H]⁺. Purity (214 nm): 100.0%; t_{R}=0.88 min.

To a solution of tert-butyl 3-(4-(1H-benzo[d]imidazol-4-yl)piperazin-1-yl)azetidine-1-carboxylate (250 mg, 0.7 mmol) in dry THF (3 mL) at 0° C. was added LAH (171 mg, 4.5 mmol) and the mixture was stirred at RT for 2 h and then Na₂SO₄—H₂O was added at 0° C. and stirred at RT for 1 h. The mixture was filtered and concentrated in vacuo and purified by silica gel column chromatography (DCM/MeOH=4/1) to give 4-(4-(1-methylazetidin-3-yl)-piperazin-1-yl)-1H-benzo[d]imidazole (30 mg, 15.8%) as a yellow oil. LC-MS m/z: 272.1 [M+H]⁺. Purity (214 nm): 100.0%, t_{R}=1.16 min.

Following general procedure C, 4-(4-(1-methylazetidin-3-yl)piperazin-1-yl)-1H-benzo[d]imidazole (30 mg, 0.1 mmol) and 3-methylbutan-1-amine (17 mg, 0.2 mmol) afforded the title compound (2.5 mg, 5.9%) as a white solid. ¹H NMR (400 MHz, CDCl₃) δ 8.39 (s, 1H), 7.37 (d, J=8.1 Hz, 1H), 7.32 (d, J=7.9 Hz, 1H), 6.74 (d, J=7.9 Hz, 1H), 6.02 (bs, 1H), 4.16 (t, J=7.8 Hz, 2H), 3.62-3.43 (m, 8H), 3.36 (q, J=7.1 Hz, 1H), 2.74 (s, 3H), 2.66-2.56 (m, 4H), 1.76 (dq, J=13.7, 6.9 Hz, 1H), 1.62 (q, J=7.1 Hz, 2H), 1.01 (d, J=6.6 Hz, 6H). LC-MS m/z: 385.4 [M+H]⁺. HPLC Purity (214 nm): 100%; t_{R}=6.20 min.

### Example 205—N-iso-Pentyl-4-(3-(4-methylpiperazin-1-yl)-azetidine-1-yl)-1H-benzo[d]imidazole-1-carboxamide

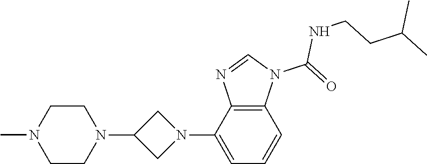 [SMILES: CC(C)CCNC(=O)n1cnc2c(N3CC(N4CCN(C)CC4)C3)cccc21]

Following general procedure E, 3-fluoro-2-nitroaniline (885 mg, 5.7 mmol) and 1-(azetidin-3-yl)-4-methylpiperazine (1.1 g, 7.1 mmol) afforded 3-(3-(4-methylpiperazin-1-yl)azetidin-1-yl)-2-nitroaniline (300 mg, 14.3%) as a red solid. LC-MS m/z: 292.2 [M+H]⁺. Purity (254 nm): 100.0%; t_{R}=0.54 min.

Following general procedure F, 3-(3-(4-methylpiperazin-1-yl)azetidin-1-yl)-2-nitroaniline (300 mg, 1.0 mmol) afforded crude 3-(3-(4-methylpiperazin-1-yl)-azetidin-1-yl)benzene-1,2-diamine (250 mg, 92.9%) as a red oil which was used directly in the next step. LC-MS m/z: 262.2 [M+H]⁺. HPLC Purity (254 nm): 96.94%; t_{R}=1.34 min.

Following general procedure G (method B), 3-(3-(4-methylpiperazin-1-yl)azetidin-1-yl)benzene-1,2-diamine (250 mg, 0.96 mmol) afforded crude 4-(3-(4-methylpiperazin-1-yl)azetidin-1-yl)-1H-benzo[d]-imidazole (240 mg, 92.3%) as a red oil which was used directly in the next step. LC-MS m/z: 272.2 [M+H]⁺. Purity (254 nm): 78.99%; t_{R}=1.41 min.

Following general procedure C, 4-(3-(4-methylpiperazin-1-yl)azetidin-1-yl)-1H-benzo[d]imidazole (240 mg, 0.89 mmol) and 3-methylbutan-1-amine (77 mg, 0.89 mmol) afforded the title compound (165.9 mg, 48.8%) as a white solid. ¹H NMR (400 MHz, CDCl₃) δ 8.31 (s, 1H), 7.2 (t, J=8.0 Hz, 1H), 6.98 (d, J=8.2 Hz, 1H), 6.28 (d, J=7.9 Hz, 1H), 5.73 (bs, 1H), 4.39 (t, J=7.4 Hz, 2H), 4.13-4.06 (m, 2H), 3.59-3.53 (m, 2H), 3.42 (q, J=6.0 Hz, 1H), 2.58 (bs, 8H), 2.39 (s, 3H), 1.74 (sept, J=6.4 Hz, 1H), 1.62 (q, J=6.8 Hz, 2H), 1.01 (d, J=6.6 Hz, 6H). LC-MS m/z: 385.4 [M+H]⁺. HPLC Purity (214 nm): 100%; t_{R}=6.55 min.

### Example 206—4-(4-Cyclopropyl-3-oxopiperazin-1-yl)-N-isopentyl-1H-benzo[d]imidazole-1-carboxamide

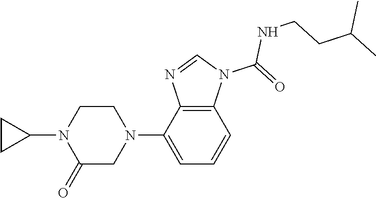 [SMILES: CC(C)CCNC(=O)n1cnc2c(N3CCN(C4CC4)C(=O)C3)cccc21]

Following general procedure E, 3-fluoro-2-nitroaniline (375 mg, 2.4 mmol) and 1-cyclopropylpiperazin-2-one (420 mg, 3.0 mmol) afforded 4-(3-amino-2-nitrophenyl)-1-cyclopropylpiperazin-2-one (320 mg, 38.6%) as a red solid. LC-MS m/z: 277.1 [M+H]⁺. Purity (214 nm): 83.49%; t_{R}=1.06 min.

Following general procedure F, 4-(3-amino-2-nitrophenyl)-1-cyclopropylpiperazin-2-one (320 mg, 1.0 mmol) afforded crude 1-cyclopropyl-4-(2,3-diaminophenyl)piperazin-2-one (250 mg, 87.7%) as a red oil which was used directly in the next step. LC-MS m/z: 247.1 [M+H]⁺. HPLC Purity (214 nm): 73.02%; t_{R}=0.79 min.

Following general procedure G (method B), 1-cyclopropyl-4-(2,3-diaminophenyl) piperazin-2-one (250 mg, 1.0 mmol) afforded crude 4-(1H-benzo[d]imidazol-4-yl)-1-cyclopropyl piperazin-2-one (170 mg, 65.4%) as a red oil. LC-MS m/z: 257.2 [M+H]⁺. Purity (214 nm): 68.87%; t_{R}=0.81 min.

Following general procedure C, 4-(1H-benzo[d]imidazol-4-yl)-1-cyclopropylpiperazin-2-one (170 mg, 0.67 mmol) and 3-methylbutan-1-amine (58 mg, 0.67 mmol) afforded the title compound (33.9 mg, 13.8%) as a white solid. ¹H NMR (400 MHz, CDCl₃) δ 8.37 (s, 1H), 7.38-7.29 (m, 2H), 6.69 (d, J=7.4 Hz, 1H), 5.85 (bs, 1H), 4.08 (s, 2H), 4.01 (t, J=6.2 Hz, 2H), 3.60-3.52 (m, 4H), 2.89-2.81 (m, 1H), 1.80-1.54 (m, 3H), 1.02 (d, J=6.6 Hz, 6H), 0.89 (d, J=5.5 Hz, 2H), 0.79-0.69 (m, 2H). LC-MS m/z: 370.0 [M+H]⁺. HPLC Purity (214 nm): 100%; t_{R}=8.68 min.

### Example 207—N-iso-Pentyl-4-(4-isopropylpiperazin-1-yl)-1H-benzo[d]imidazole-1-carboxamide

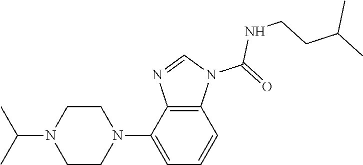 [SMILES: CC(C)CCNC(=O)n1cnc2c(N3CCN(C(C)C)CC3)cccc21]

Following general procedure E, 3-fluoro-2-nitroaniline (500 mg, 3.20 mmol) and 1-iso-propylpiperazine hydrochloride (791 mg, 4.80 mmol) afforded 3-(4-iso-propylpiperazin-1-yl)-2-nitroaniline (830 mg, 98%) as an orange oil. LC-MS m/z: 265.2 [M+H]⁺. Purity (214 nm): 100%; t_{R}=1.64 min.

Following general procedure F, 3-(4-iso-propylpiperazin-1-yl)-2-nitroaniline (830 mg, 3.14 mmol) afforded crude 3-(4-iso-propylpiperazin-1-yl)benzene-1,2-diamine (640 mg) as an orange solid which was used directly in the next step. LC-MS m/z: 235.2 [M+H]⁺. Purity (254 nm): 97.56%; t_{R}=1.29 min.

Following general procedure G (method B), 3-(4-iso-propylpiperazin-1-yl)benzene-1,2-diamine (640 mg, 2.73 mmol) afforded crude 4-(4-iso-propylpiperazin-1-yl)-1H-benzo[d]imidazole (560 mg) as a light brown solid which was used directly in the next step. LC-MS m/z: 245.2 [M+H]⁺. Purity (214 nm): 100%; t_{R}=1.32 min.

Following general procedure C, 4-(4-isopropylpiperazin-1-yl)-1H-benzo[d]imidazole (200 mg, 0.82 mmol) and 3-methylbutan-1-amine (71 mg, 0.82 mmol) afforded the title compound (103.7 mg, 30.6%) as a white solid. ¹H NMR (400 MHz, CDCl₃) δ 8.35 (s, 1H), 7.30-7.27 (m, 2H), 6.75 (dd, J=6.5, 2.3 Hz, 1H), 5.75 (t, J=4.8 Hz, 1H), 3.65-3.52 (m, 6H), 2.89-2.69 (m, 5H), 1.77-1.69 (m, 1H), 1.59 (dd, J=14.8, 7.1 Hz, 2H), 1.13 (d, J=6.5 Hz, 6H), 0.99 (d, J=6.6 Hz, 6H). LC-MS m/z: 358.2 [M+H]⁺. HPLC Purity (214 nm): 100%; t_{R}=6.81 min.

### Example 208—(4-(4-Ethylpiperazin-1-yl)-N-isopentyl-1H-benzo[d]imidazole-1-carboxamide

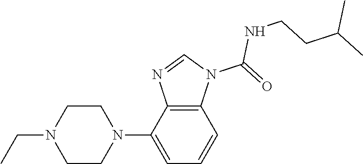 [SMILES: CCN1CCN(c2cccc3c2ncn3C(=O)NCCC(C)C)CC1]

Following general procedure E, 1-ethylpiperazine (1.3 g, 11.3 mmol) and 3-fluoro-2-nitroaniline (2.6 g, 17.0 mmol) afforded 3-(4-ethylpiperazin-1-yl)-2-nitroaniline (1.2 g) as a white solid. LC-MS m/z: 251.7 [M+H]⁺.

Following general procedure F, 3-(4-ethylpiperazin-1-yl)-2-nitroaniline (1.2 g, 4.8 mmol) afforded crude 3-(4-ethylpiperazin-1-yl)benzene-1,2-diamine (600 mg) as a white solid which was used directly in the next step. LC-MS m/z: 221.7 [M+H]⁺.

Following general procedure G (method B), 3-(4-ethylpiperazin-1-yl)benzene-1,2-diamine (600 mg, 2.7 mmol) afforded crude 4-(4-ethylpiperazin-1-yl)-1H-benzo[d]imidazole (500 mg) as a white solid. LC-MS m/z: 231.7 [M+H]⁺.

Following general procedure C, 4-(4-ethylpiperazin-1-yl)-1H-benzo-[d]imidazole (250 mg, 1.1 mmol) and 3-methylbutan-1-amine (113 mg, 1.3 mmol) afforded the title compound (72.8 mg, 19.6%) as a white solid. ¹H NMR (400 MHz, CDCl₃) δ 8.68 (s, 1H), 7.60 (d, J=8.2 Hz, 1H), 7.26-7.22 (m, 2H), 6.68 (d, J=7.9 Hz, 1H), 3.79 (bs, 4H), 3.50 (dd, J=14.7, 5.9 Hz, 2H), 3.30 (bs, 4H), 3.03 (q, J=7.3 Hz, 2H), 1.72 (dq, J=13.1, 6.5 Hz, 1H), 1.61 (dd, J=14.8, 7.1 Hz, 2H), 1.42 (t, J=7.3 Hz, 3H), 0.98 (d, J=6.5 Hz, 6H). LC-MS m/z: 344.1 [M+H]⁺. Purity (214 nm): 100%; t_{R}=6.71 min.

### Example 209—N-iso-Pentyl-4-(6-oxohexahydropyrrolo[1,2-a]pyrazin-2(1H)-yl)-1H-benzo[d]imidazole-1-carboxamide

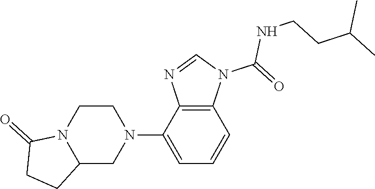 [SMILES: CC(C)CCNC(=O)n1cnc2c(N3CCN4C(=O)CCC4C3)cccc21]

Following general procedure E, 3-fluoro-2-nitroaniline (500 mg, 3.20 mmol) and hexahydropyrrolo[1,2-a]pyrazin-6(2H)-one (539 mg, 3.84 mmol) afforded 2-(3-amino-2-nitrophenyl)hexahydropyrrolo[1,2-a]pyrazin-6(2H)-one (410 mg, 46.3%) as a light brown semisolid. LC-MS m/z: 277.2 [M+H]⁺. Purity (214 nm): 100%; t_{R}=1.40 min.

Following general procedure F, 2-(3-amino-2-nitrophenyl)hexahydropyrrolo[1,2-a]pyrazin-6(2H)-one (410 mg, 1.48 mmol) afforded crude 2-(2,3-diaminophenyl)hexahydro pyrrolo[1,2-a]-pyrazin-6(2H)-one (370 mg) as a gray solid which was used directly in the next step. LC-MS m/z: 247.2 [M+H]⁺. Purity (254 nm): 94.78%; t_{R}=1.27 min.

Following general procedure G (method B), 2-(2,3-diaminophenyl)hexahydropyrrolo[1,2-a]pyrazin-6(2H)-one (370 mg, 1.50 mmol) afforded crude 2-(1H-benzo[d]imidazol-4-yl)-hexahydropyrrolo[1,2-a]pyrazin-6(2H)-one (390 mg) as a light brown solid which was used directly in the next step. LC-MS m/z: 257.2 [M+H]⁺. Purity (214 nm): 100%; t_{R}=0.80 min.

Following general procedure C, 2-(1H-benzo[d]imidazol-4-yl)hexahydropyrrolo[1,2-a]pyrazin-6(2H)-one (200 mg, 0.78 mmol) and 3-methylbutan-1-amine (68 mg, 0.78 mmol) afforded the title compound (115.5 mg, 40.1%) as a white solid. ¹H NMR (400 MHz, CDCl₃) δ 8.38 (s, 1H), 7.38 (d, J=7.5 Hz, 1H), 7.30 (t, J=8.0 Hz, 1H), 6.73 (t, J=10.2 Hz, 1H), 5.94 (bs, 1H), 4.35 (dd, J=11.6, 2.1 Hz, 1H), 4.18-4.05 (m, 2H), 3.97 (dtd, J=10.9, 7.3, 3.8 Hz, 1H), 3.61-3.47 (m, 2H), 3.28-3.13 (m, 1H), 2.78 (td, J=11.9, 3.5 Hz, 1H), 2.49 (ddd, J=16.5, 12.2, 7.8 Hz, 3H), 2.36-2.22 (m, 1H), 1.77-1.65 (m, 2H), 1.60 (dd, J=14.8, 7.2 Hz, 2H), 0.99 (d, J=6.6 Hz, 6H). LC-MS m/z: 370.1 [M+H]⁺. HPLC Purity (214 nm): 96.76%; t_{R}=7.90 min.

### Example 210—6-Cyano-N-iso-pentyl-2-methoxy-4-(4-methylpiperazin-1-yl)-1H-benzo[d]imidazole-1-carboxamide

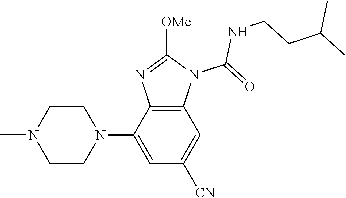 [SMILES: COc1nc2c(N3CCN(C)CC3)cc(C#N)cc2n1C(=O)NCCC(C)C]

A solution of 4-amino-3-nitrobenzonitrile (5.0 g, 30.7 mmol) and NBS (21.8 g, 122.8 mmol) in MeCN (50 mL) was stirred at RT for 16 h. Then the reaction mixture was concentrated and the residue was purified by silica gel column chromatography (PE:EA=4:1) to give 4-amino-3-bromo-5-nitrobenzonitrile (5.0 g, 71.0%) as a yellow solid. LC-MS m/z: 242.0 [M+H]⁺. Purity (214 nm): 98%; t_{R}=1.50 min.

A suspension of 4-amino-3-bromo-5-nitrobenzonitrile (1.0 g, 4.14 mmol), Zn (1.07 g, 16.4 mmol) and NH₄Cl (1.09 g, 20.0 mmol) in EtOH was stirred at 80° C. for 4 h. The reaction mixture was filtered and the filtrate was concentrated. The residue was poured into water (20 mL) and extracted with DCM (50 mL×2). The combined organic layers were washed with brine (50 mL×1), dried over Na₂SO₄, filtered and concentrated to give 3,4-diamino-5-bromobenzonitrile (400 mg, 43.7) as a brown solid. LC-MS m/z: 214.0 [M+H]⁺. Purity (214 nm): 80%; t_{R}=1.48 min.

Following general procedure G, 3,4-diamino-5-bromobenzonitrile (1.1 g, 5.19 mmol) after silica gel column chromatography (EA:PE=1:1) afforded 4-bromo-2-methoxy-1H-benzo[d]imidazole-6-carbonitrile (1.1 g, 85%) as a white solid. LC-MS m/z: 253.9 [M+H]⁺. Purity (254 nm): 97%; t_{R}=1.74 min.

To a solution of 4-bromo-2-methoxy-1H-benzo[d]imidazole-6-carbonitrile (1.0 g, 3.96 mmol) in DMF (10 mL) was added NaH (371 mg, 7.94 mmol) at 0° C. under N₂. The mixture was then stirred at 0° C. for 10 min, SEM-Cl (986 mg, 5.94 mmol) was added and the resulting mixture was stirred at RT for 2 h. The reaction mixture was poured into water (50 mL) and extracted with EA (50 mL×3), washed with brine (30 mL), dried over Na₂SO₄, filtered and concentrated to give a residue which was purified by silica gel column chromatography (EA:PE=1:2) to give 4-bromo-2-methoxy-1-((2-(trimethylsilyl)ethoxy)methyl)-1H-benzo[d]imidazole-6-carbonitrile (1.1 g, 73.3%) as a white solid. LC-MS m/z: 384.0 [M+H]⁺. Purity (214 nm): 99%; t_{R}=2.16 min.

Following general procedure H, 4-bromo-2-methoxy-1-((2-(trimethylsilyl)ethoxy)methyl)-1H-benzo[d]imidazole-6-carbonitrile (525 mg, 1.38 mmol) and 1-methylpiperazine (276 mg, 2.76 mmol) afforded 2-methoxy-4-(4-methylpiperazin-1-yl)-1-((2-(trimethylsilyl)ethoxy)-methyl)-1H-benzo[d]imidazole-6-carbonitrile (270 mg, 48.8%) as a brown oil. LC-MS m/z: 402.0 [M+H]⁺. Purity (214 nm): 85%; t_{R}=1.67 min.

A solution of 2-methoxy-4-(4-methylpiperazin-1-yl)-1-((2-(trimethylsilyl)ethoxy)-methyl)-1H-benzo[d]imidazole-6-carbonitrile (270 mg, 0.67 mmol) in TFA (4 mL) was stirred at RT for 1 h, and the resulting mixture was concentrated and dissolved in methanolic NH₃ (4 mL). The reaction mixture was stirred at RT for 10 min, concentrated and purified by silica gel column chromatography (DCM:MeOH=6:1) and Prep-HPLC (MeCN/NH₄HCO₃) to give 2-methoxy-4-(4-methylpiperazin-1-yl)-1H-benzo[d]imidazole-6-carbonitrile (128 mg, 70.0%) as a white solid. LC-MS m/z: 272.0 [M+H]⁺. Purity (214 nm): 99%; t_{R}=1.41 min.

Following general procedure C, 2-methoxy-4-(4-methylpiperazin-1-yl)-1H-benzo[d]imidazole-6-carbonitrile (125 mg, 0.46 mmol) and 3-methylbutan-1-amine (60 mg, 0.69 mmol) afforded the title compound (16.8 mg, 9.5%) as a white solid. ¹H NMR (400 MHz, CDCl₃) δ 8.19 (s, 1H), 6.92 (s, 1H), 6.89 (t, J=5.2 Hz, 1H), 4.35 (s, 3H), 3.54 (bs, 4H), 3.44 (q, J=6.4 Hz, 2H), 2.78 (bs, 4H), 2.46 (s, 3H), 1.73-1.64 (m, 1H), 1.54 (q, J=7.2 Hz, 2H), 0.98 (d, J=6.4 Hz, 6H). LC-MS m/z: 385.2 [M+H]⁺. HPLC Purity (214 nm): 99%; t_{R}=7.00 min.

### Example 211—N-iso-Pentyl-2-methoxy-7-morpholino-3H-imidazo[4,5-c]pyridine-3-carboxamide

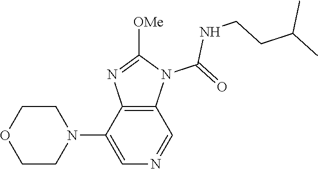 [SMILES: COc1nc2c(N3CCOCC3)cncc2n1C(=O)NCCC(C)C]

To a mixture of 3-nitropyridin-4-amine (4.0 g, 28.8 mmol) in CH₃CN (30 mL) was added NBS (6.2 g, 34.6 mmol) and the mixture was stirred at RT for 12 h under N₂. The reaction was concentrated in vacuo and purified by silica gel column chromatography (DCM:MeOH=30:1) to give 3-bromo-5-nitropyridin-4-amine (2.7 g, 42.9%) as a yellow solid. LC-MS m/z: 218.0 [M+H]⁺. Purity (214 nm): 95.6%; t_{R}=0.85 min.

A solution of 3-bromo-5-nitropyridin-4-amine (2.2 g, 10 mmol) in morpholine (20 mL) was stirred at 140° C. in a sealed tube for 12 h. Then concentrated in vacuo and purified by silica gel column chromatography (DCM:MeOH=30:1) to give 3-morpholino-5-nitropyridin-4-amine (1.9 g, 84.4%) as a yellow solid. LC-MS m/z: 225.0 [M+H]⁺. Purity (214 nm): 89.7%; t_{R}=1.54 min.

Following general procedure F, 3-morpholino-5-nitropyridin-4-amine (1.12 g, 5.0 mmol) afforded crude 5-morpholinopyridine-3,4-diamine (1.9 g) as a yellow oil which was used directly in the next step. LC-MS m/z: 195.1 [M+H]⁺. Purity (214 nm): 76.7%; t_{R}=0.90 min.

Following general procedure G (method A), 5-morpholinopyridine-3,4-diamine (1.87 g, 10 mmol) after silica gel column chromatography (DCM:MeOH=20:1) afforded 4-(2-methoxy-3H-imidazo[4,5-c]pyridin-7-yl)morpholine (95 mg) as a yellow solid. LC-MS m/z: 235.1 [M+H]⁺. Purity (214 nm): 896.7%; t_{R}=1.36 min.

Following general procedure C, 4-(2-methoxy-3H-imidazo[4,5-c]-pyridin-7-yl) morpholine (121 mg, 0.5 mmol) and 3-methylbutan-1-amine (40.0 mg, 0.5 mmol) afforded the title compound (15.2 mg, 8.7%) as a yellow solid. ¹H NMR (400 MHz, CDCl₃) δ 8.98 (d, J=0.4 Hz, 1H), 6.79 (t, J=5.2 Hz, 1H), 6.76 (s, 1H), 4.34 (s, 3H), 3.88 (t, J=4.8 Hz, 4H), 3.49-3.43 (m, 6H), 1.79-1.61 (m, 1H), 1.49 (dt, J=7.6, 7.2 Hz, 2H), 0.95 (d, J=6.4 Hz, 6H). LC-MS m/z: 348.1 [M+H]⁺. HPLC Purity (214 nm): 100%; t_{R}=6.41 min.

### Example 212—6-Fluoro-N-iso-pentyl-2-methoxy-4-morpholino-1H-benzo[d]imidazole-1-carboxamide

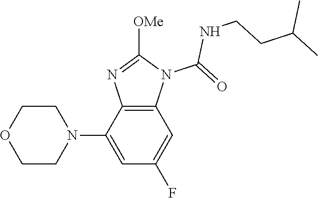 [SMILES: COc1nc2c(N3CCOCC3)cc(F)cc2n1C(=O)NCCC(C)C]

Following general procedure E, 3,5-difluoro-2-nitroaniline (522 mg, 3.0 mmol) and morpholine (313 mg, 3.6 mmol) afforded crude 5-fluoro-3-morpholino-2-nitroaniline (720 mg, 100%) as a yellow solid which was used directly in the next step. LC-MS m/z: 242.1 [M+H]⁺. Purity (214 nm): 94%; t_{R}=1.61 min.

Following general procedure F, 5-fluoro-3-morpholino-2-nitroaniline (720 mg, 2.98 mmol) afforded 5-fluoro-3-morpholinobenzene-1,2-diamine (550 mg, 87%) as a yellow solid which was used directly in the next step. LC-MS m/z: 212.1 [M−H]⁺. Purity (214 nm): 93%; t_{R}=1.43 min.

Following general procedure G (method A), 5-fluoro-3-morpholinobenzene-1,2-diamine (550 mg, 2.6 mmol) afforded 4-(6-fluoro-2-methoxy-1H-benzo[d]imidazol-4-yl)-morpholine (700 mg, 100%) as a yellow solid. LC-MS m/z: 252.1 [M−H]⁻. Purity (254 nm): 57%; t_{R}=1.55 min.

Following general procedure C, 4-(6-fluoro-2-methoxy-1H-benzo[d]-imidazol-4-yl)morpholine (250 mg, 1.0 mmol) and 3-methylbutan-1-amine (105 mg, 1.2 mmol) afforded the title compound (28 mg, 23%) as a white solid. ¹H NMR (400 MHz, CDCl₃) δ 7.59 (dd, J=9.2, 2.0 Hz, 1H), 6.94 (t, J=5.2 Hz, 1H), 6.47 (dd, J=12.0, 2.4 Hz, 1H), 3.97 (t, J=4.4 Hz, 4H), 3.48-3.41 (m, 6H), 1.72-1.67 (m, 1H), 1.62 (s, 3H), 1.56 (q, J=7.2 Hz, 2H), 0.99 (d, J=6.4 Hz, 6H). LC-MS m/z: 365.2 [M+H]⁺. HPLC Purity (214 nm): 98%; t_{R}=11.18 min.

### Example 213a and 213b—2-Methoxy-6-morpholino-N-(3-phenylpropyl)-1H-imidazo[4,5-c]pyridine-1-carboxamide and 2-methoxy-6-morpholino-N-(3-phenylpropyl)-3H-imidazo[4,5-c]pyridine-3-carboxamide

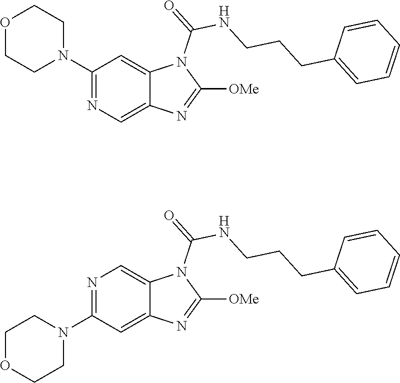 [SMILES: COc1nc2cnc(N3CCOCC3)cc2n1C(=O)NCCCc1ccccc1 ; COc1nc2cc(N3CCOCC3)ncc2n1C(=O)NCCCc1ccccc1]

Following general procedure E, 2-chloro-5-nitropyridin-4-amine (5 g, 28.81 mmol) and morpholine (3.8 g, 43.213 mmol) afforded crude 2-morpholino-5-nitropyridin-4-amine (10 g) as a light brown solid which was used directly in the next step. LC-MS m/z: 225.1 [M+H]⁺. Purity (254 nm): 90.2%; t_{R}=1.59 min.

Following general procedure F, 2-morpholino-5-nitropyridin-4-amine (10 g, 44.60 mmol) afforded crude 6-morpholinopyridine-3,4-diamine (8.5 g) as a brown solid which was used directly in the next step. LC-MS m/z: 195.2 [M+H]⁺. Purity (254 nm): 91.2%; t_{R}=0.85 min.

Following general procedure G (method A), 6-morpholinopyridine-3,4-diamine (4.5 g, 23.1 mmol) afforded 4-(2-methoxy-3H-imidazo[4,5-c]pyridin-6-yl)morpholine (370 mg, 4%) as a light yellow solid. LC-MS m/z: 235.1 [M+H]⁺. Purity (214 nm): 99.37%; t_{R}=1.16 min.

Following general procedure C, 4-(2-methoxy-3H-imidazo[4,5-c]pyridin-6-yl)morpholine (170 mg, 0.726 mmol) and 3-phenylpropan-1-amine (98 mg, 0.726 mmol) afforded 2-methoxy-6-morpholino-N-(3-phenylpropyl)-1H-imidazo[4,5-c]pyridine-1-carboxamide (42.1 mg, 14.7%) and 2-methoxy-6-morpholino-N-(3-phenylpropyl)-3H-imidazo[4,5-c]pyridine-3-carboxamide (35.1 mg, 12.2%) both as white solids.

2-methoxy-6-morpholino-N-(3-phenylpropyl)-1H-imidazo[4,5-c]pyridine-1-carboxamide: ¹H NMR (400 MHz, CDCl₃) δ 8.43 (d, J=0.8 Hz, 1H), 7.45 (s, 1H), 7.33-7.27 (m, 2H), 7.25-7.20 (m, 3H), 6.93 (t, J=5.5 Hz, 1H), 4.29 (s, 3H), 3.86 (t, J=4.8 Hz, 4H), 3.51 (t, J=5.2 Hz, 4H), 3.46 (q, J=6.0 Hz, 6H), 2.73 (t, J=7.5 Hz, 2H), 2.02 (p, J=7.2 Hz, 2H). LC-MS m/z: 396.1 [M+H]⁺. HPLC Purity (214 nm): 99%; t_{R}=6.47 min.

2-methoxy-6-morpholino-N-(3-phenylpropyl)-3H-imidazo[4,5-c]pyridine-3-carboxamide: ¹H NMR (400 MHz, CDCl₃) δ 8.96 (s, 1H), 7.32-7.27 (m, 2H), 7.23-7.16 (m, 3H), 6.78 (t, J=5.4 Hz, 1H), 6.75 (s, 1H), 4.31 (s, 3H), 3.89 (t, J=4.4 Hz, 4H), 3.51-3.44 (m, 6H), 2.73 (t, J=7.5 Hz, 2H), 2.00 (p, J=7.4 Hz, 2H). LC-MS m/z: 396.1 [M+H]⁺. HPLC Purity (214 nm): 99%; t_{R}=6.26 min.

### Example 214—5-Fluoro-2-methoxy-6-morpholino-N-(4-phenylbutyl)-1H-benzo[d]imidazole-1-carboxamide

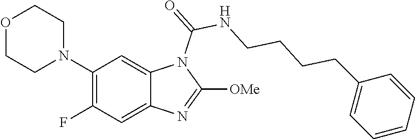 [SMILES: COc1nc2cc(F)c(N3CCOCC3)cc2n1C(=O)NCCCCc1ccccc1]

Following general procedure E, 4,5-difluoro-2-nitroaniline (3.0 g, 17.24 mmol) and morpholine (2.2 g, 25.86 mmol) afforded 5-fluoro-4-morpholino-2-nitroaniline (4 g, 96%) as an orange solid. LC-MS m/z: 242.0 [M+H]⁺. Purity (214 nm): 99%; t_{R}=1.83 min.

Following general procedure F, 5-fluoro-4-morpholino-2-nitroaniline (4 g, 16.60 mmol) afforded 4-fluoro-5-morpholinobenzene-1,2-diamine (1.7 g, 49%) as an orange solid. LC-MS m/z: 212.2 [M+H]⁺. Purity (214 nm): 89%; t_{R}=1.09 min.

Following general procedure G (method A), 4-fluoro-5-morpholinobenzene-1,2-diamine (680 mg, 3.22 mmol) afforded 4-(6-fluoro-2-methoxy-1H-benzo[d]imidazol-5-yl)morpholine (600 mg, 74%) as a brown solid. LC-MS m/z: 252.1 [M+H]⁺. Purity (214 nm): 89%; t_{R}=1.31 min.

Following general procedure C, 4-(6-fluoro-2-methoxy-1H-benzo[d]imidazol-5-yl)morpholine (200 mg, 0.80 mmol) and 4-phenylbutan-1-amine (178 mg, 1.20 mmol) afforded the title compound (89.5 mg, 26%) as a white solid. ¹H NMR (400 MHz, CDCl₃) δ 7.87 (d, J=8.1 Hz, 1H), 7.29 (t, J=7.5 Hz, 2H), 7.22-7.17 (m, 4H), 6.95 (t, J=5.4 Hz, 1H), 4.28 (s, 3H), 3.94-3.84 (m, 4H), 3.45 (q, J=6.6 Hz, 2H), 3.15-3.04 (m, 4H), 2.69 (t, J=7.1 Hz, 2H), 1.78-1.64 (m, 4H). LC-MS m/z: 427.1 [M+H]⁺. Purity (214 nm): 99%; t_{R}=9.46 min.

### Example 215—6-Fluoro-2-methoxy-5-morpholino-N-(4-phenylbutyl)-1H-benzo[d]imidazole-1-carboxamide

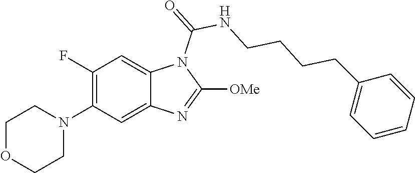 [SMILES: COc1nc2cc(N3CCOCC3)c(F)cc2n1C(=O)NCCCCc1ccccc1]

Following general procedure C, 4-(6-fluoro-2-methoxy-1H-benzo[d]-imidazol-5-yl)morpholine (200 mg, 0.80 mmol) and 4-phenylbutan-1-amine (178 mg, 1.20 mmol) afforded the title compound (49.8 mg, 15%) as a white solid. ¹H NMR (400 MHz, CDCl₃) δ 7.90 (d, J=12.7 Hz, 1H), 7.33-7.27 (m, 2H), 7.21-7.16 (m, 3H), 7.11 (d, J=7.7 Hz, 1H), 6.90 (t, J=5.5 Hz, 1H), 4.28 (s, 3H), 3.95-3.85 (m, 4H), 3.44 (dd, J=12.6, 6.7 Hz, 2H), 3.11-3.02 (m, 4H), 2.68 (t, J=7.2 Hz, 2H), 1.77-1.64 (m, 4H). LC-MS m/z: 427.1 [M+H]⁺. Purity (214 nm): 99%; t_{R}=9.50 min.

### Example 216—N-(3-Phenylpropyl)-4-(2-oxa-7-azaspiro[3.5]nonan-7-yl)-1H-benzo[d]imidazole-1-carboxamide

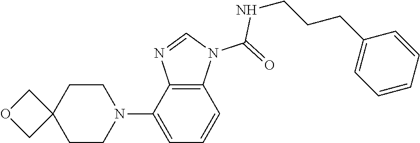 [SMILES: O=C(NCCCc1ccccc1)n1cnc2c(N3CCC4(CC3)COC4)cccc21]

Following general procedure E, 3-fluoro-2-nitroaniline (400 mg, 2.56 mmol) and 2-oxa-7-azaspiro[3.5]nonane (441 mg, 2.56 mmol) afforded 2-nitro-3-(2-oxa-7-azaspiro[3.5]nonan-7-yl)aniline (400 mg, 59%) as a yellow solid. LC-MS m/z: 264.0 [M+H]⁺. Purity (214 nm): >99%; t_{R}=1.77 min.

Following general procedure F, 2-nitro-3-(2-oxa-7-azaspiro[3.5]nonan-7-yl)aniline (400 mg, 1.5 mmol) afforded 3-(2-oxa-7-azaspiro[3.5]nonan-7-yl)benzene-1,2-diamine (317 mg, 90%) as a yellow solid. LC-MS m/z: 234.2 [M+H]⁺. Purity (254 nm): >99%; t_{R}=0.71 min.

Following general procedure G (method A), 3-(2-oxa-7-azaspiro[3.5]nonan-7-yl)benzene-1,2-diamine (180 mg, 0.77 mmol) afforded 7-(1H-benzo[d]imidazol-4-yl)-2-oxa-7-azaspiro[3.5]nonane (180 mg, 96%) as a yellow solid which was used directly in the next step. LC-MS m/z: 244.2 [M+H]⁺. Purity (254 nm): >90%; t_{R}=1.46 min.

Following general procedure C, 7-(1H-benzo[d]imidazol-4-yl)-2-oxa-7-azaspiro[3.5]nonane (90 mg, 0.37 mmol) and 3-phenylpropan-1-amine (50 mg, 0.37 mmol) afforded the title compound (48.3 mg, 32.3%) as a white solid. ¹H NMR (400 MHz, CDCl₃) δ 8.20 (s, 1H), 7.38-7.12 (m, 7H), 6.73 (d, J=6.9 Hz, 1H), 5.71 (bs, 1H), 4.51 (s, 4H), 3.55 (dd, J=13.0, 6.5 Hz, 2H), 3.44-3.31 (m, 414), 2.77 (t, J=7.4 Hz, 2H), 2.16-2.13 (m, 4H) 2.08 (p, J=7.2 Hz, 2H). LC-MS m/z: 405.0 [M+H]⁺. Purity (214 nm): >99%; t_{R}=1.88 min.

### Example 217—N-(4-Phenylbutyl)-4-(2-oxa-7-azaspiro[3.5]nonan-7-yl)-1H-benzo[d]imidazole-1-carboxamide

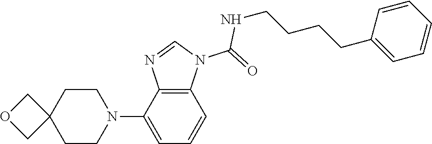 [SMILES: O=C(NCCCCc1ccccc1)n1cnc2c(N3CCC4(CC3)COC4)cccc21]

Following general procedure C, 7-(1H-benzo[d]imidazol-4-yl)-2-oxa-7-azaspiro[3.5]nonane (90 mg, 0.37 mmol) and 4-phenylbutan-1-amine (55 mg, 0.37 mmol) afforded the title compound (37.8 mg, 24.4%) as a white solid. ¹H NMR (400 MHz, CDCl₃) δ 8.32 (s, 1H), 7.35-7.23 (m, 4H), 7.20 (t, J=7.5 Hz, 3H), 6.74 (dd, J=8.8, 4.5 Hz, 1H), 5.77 (s, 1H), 4.51 (s, 4H), 3.52 (q, J=6.2 Hz, 2H), 3.44-3.29 (m, 4H), 2.70 (t, J=6.8 Hz, 2H), 2.22-2.04 (m, 4H), 1.74 (d, J=5.0 Hz, 4H). LC-MS m/z: 419.0 [M+H]⁺. Purity (214 nm): >99%; t_{R}=7.61 min.

### Example 218—4-(4-Acetylpiperazin-1-yl)-N-(3-phenylpropyl)-1H-benzo[d]imidazole-1-carboxamide

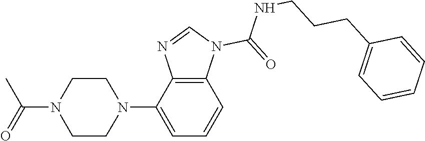 [SMILES: CC(=O)N1CCN(c2cccc3c2ncn3C(=O)NCCCc2ccccc2)CC1]

Following general procedure C, 1-(4-(1H-benzo[d]imidazol-4-yl)-piperazin-1-yl)ethan-1-one (244 mg, 1.0 mmol) and 3-phenylpropan-1-amine (134 mg, 1.0 mmol) afforded the title compound (164.8 mg, 49.6%) as a white solid. ¹H NMR (400 MHz, CDCl₃) δ 8.24 (s, 1H), 7.40-7.27 (m, 4H), 7.26-7.22 (m, 3H), 6.71 (d, J=7.6 Hz, 1H), 6.10 (t, J=5.4 Hz, 1H), 3.82 (t, J=5.2 Hz, 2H), 3.73 (t, J=4.8 Hz, 2H), 3.57 (q, J=6.0 Hz, 2H), 3.55-3.51 (m, 2H), 3.463 (t, J=4.8 Hz, 2H), 2.78 (t, J=7.4 Hz, 2H), 2.16 (s, 3H), 2.08 (p, J=7.6 Hz, 2H). LC-MS m/z: 406.2 [M+H]⁺. HPLC Purity (214 nm): 96.49%; t_{R}=7.92 min.

### Example 219—4-((2S,6R)-2,6-Dimethylmorpholino)-N-(3-phenylpropyl)-1H-benzo[d]imidazole-1-carboxamide

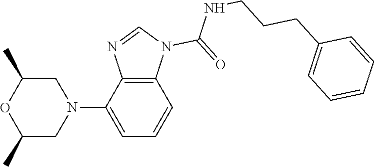 [SMILES: C[C@@H]1CN(c2cccc3c2ncn3C(=O)NCCCc2ccccc2)C[C@H](C)O1]

Following general procedure E, 3-fluoro-2-nitrobenzenamine (400.0 mg, 2.6 mmol) and (2S,6R)-2,6-dimethylmorpholine (249.2 mg, 2.2 mmol) afforded 3-((2S,6R)-2,6-dimethyl morpholino)-2-nitrobenzenamine (300.0 mg, 54.3%) as a yellow solid. LC-MS m/z: 252.2 [M+H]⁺. Purity (254 nm): 100.0%; t_{R}=1.56 min.

Following general procedure F, 3-((2S,6R)-2,6-dimethylmorpholino)-2-nitrobenzenamine (300.0 mg, 1.2 mmol) afforded 3-((2S,6R)-2,6-dimethylmorpholino)benzene-1,2-diamine (200.0 mg, 75.4%) as a brown oil which was used directly in the next step. LC-MS m/z: 222.2 [M+H]⁺. Purity (214 nm): 94.5%; t_{R}=1.64 min.

Following general procedure G (method A), 3-((2S,6R)-2,6-dimethylmorpholino) benzene-1,2-diamine (200.0 mg, 0.9 mmol) afforded (2S,6R)-4-(1H-benzo[d]imidazol-4-yl)-2,6-dimethylmorpholine (240.0 mg, 94.7%) as a yellow oil. LC-MS m/z: 232.2 [M+H]⁺. Purity (214 nm): 82.7%; t_{R}=1.54 min.

Following general procedure C, (2S,6R)-4-(1H-benzo[d]imidazol-4-yl)-2,6-dimethyl morpholine (200 mg, 0.9 mmol) and 3-phenylpropan-1-amine (148.5 mg, 1.1 mmol) afforded the title compound (277.7 mg, 81.8%) as a white solid. ¹H NMR (400 MHz, CDCl₃) δ 8.20 (s, 1H), 7.34-7.28 (m, 2H), 7.25-7.19 (m, 5H), 6.72 (d, J=7.6 Hz, 1H), 5.64 (bs, 1H), 4.08-3.97 (m, 4H), 3.56 (dd, J=13.1, 6.6 Hz, 2H), 2.78 (t, J=7.4 Hz, 2H), 2.54 (t, J=11.0 Hz, 2H), 2.06 (p, J=7.2 Hz, 2H), 1.28 (d, J=6.2 Hz, 6H). LC-MS m/z: 393.0 [M+H]⁺. HPLC Purity (214 nm): 97.97%; t_{R}=8.97 min.

### Example 220—4-((1R,5S)-3-Oxa-8-azabicyclo[3.2.1]octan-8-yl)-N-isopentyl-1H-benzo[d]imidazole-1-carboxamide

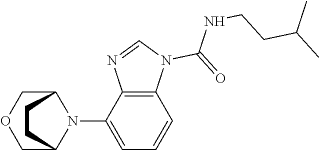

Following general procedure E, (1R,5S)-3-oxa-8-azabicyclo[3.2.1]octane hydrochloride (1.0 g, 6.67 mmol) and 3-fluoro-2-nitroaniline (1.0 g, 6.67 mmol) afforded 3-((11R,5S)-3-oxa-8-azabicyclo[3.2.1]octan-8-yl)-2-nitroaniline (917 mg, 62.5%) as a yellow oil. LC-MS m/z: 250.1 [M+H]⁺. Purity (214 nm): 90.24%; t_{R}=1.71 min.

Following general procedure F, 3-((1R,5S)-3-oxa-8-azabicyclo[3.2.1]octan-8-yl)-2-nitroaniline (917 mg, 3.68 mmol) afforded 3-((1R,5S)-3-oxa-8-azabicyclo[3.2.1]octan-8-yl)benzene-1,2-diamine (1.0 g, 124%) as a yellow solid which was used directly in the next step. LC-MS m/z: 220.1 [M+H]⁺. Purity (214 nm): 83.35%; t_{R}=1.55 min.

Following general procedure G (method B), 3-((1R,5S)-3-oxa-8-azabicyclo[3.2.1]octan-8-yl)benzene-1,2-diamine (1.0 g, 4.57 mmol) afforded (1R,5S)-8-(1H-benzo[d]imidazol-4-yl)-3-oxa-8-azabicyclo[3.2.1]octane (1.1 g, 106%) as a yellow solid which was used directly in the next step. LC-MS m/z: 230.1 [M+H]⁺. Purity (214 nm): 86.81%; t_{R}=1.52 min.

Following general procedure C, (1R,5S)-8-(1H-benzo[d]imidazol-4-yl)-3-oxa-8-azabicyclo[3.2.1]octane (230 mg, 1.0 mmol) and 3-methylbutan-1-amine (87 mg, 1.0 mmol) afforded the title compound (152.7 mg, 44.7%) as a white solid. 1H NMR (400 MHz, CDCl₃) δ 8.30 (s, 1H), 7.23 (d, J=8.1 Hz, 1H), 7.10 (d, J=7.8 Hz, 1H), 6.64 (d, J=7.9 Hz, 1H), 5.72 (bs, 1H), 4.84 (s, 2H), 3.99 (d, J=10.8 Hz, 2H), 3.69 (d, J=10.6 Hz, 2H), 3.58-3.50 (m, 2H), 2.12-2.07 (m, 2H), 1.71 (dt, J=13.2, 6.6 Hz, 1H), 1.61-1.55 (m, 4H), 0.99 (d, J=6.6 Hz, 6H). LC-MS m/z: 343.2 [M+H]⁺. HPLC Purity (214 nm): 95.03%; t_{R}=10.05 min.

### Example 221—4-(8-Oxa-3-azabicyclo[3.2.1]octan-3-yl)-N-isopentyl-1H-benzo[d]imidazole-1-carboxamide

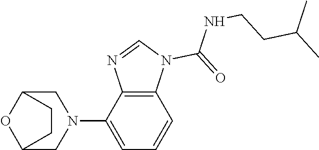 [SMILES: CC(C)CCNC(=O)n1cnc2c(N3CC4CCC(C3)O4)cccc21]

Following general procedure F, 3-(8-oxa-3-azabicyclo[3.2.1]octan-3-yl)-2-nitroaniline (300 mg, 1.20 mmol) afforded 3-(8-oxa-3-azabicyclo[3.2.1]octan-3-yl)benzene-1,2-diamine (300 mg, 100%) as a pale yellow oil. LC-MS m/z: 220.1. [M+H]⁺.

Following general procedure G (method A), 3-(8-oxa-3-azabicyclo[3.2.1]octan-3-yl)benzene-1,2-diamine (300 mg, 1.37 mmol) afforded 3-(1H-benzo[d]imidazol-4-yl)-8-oxa-3-azabicyclo[3.2.1]octane (189 mg, 60.3%) as a pale yellow oil. The crude product was used directly in the next step. LC-MS m/z: 230.1. [M+H]⁺.

Following general procedure C, 3-(1H-benzo[d]imidazol-4-yl)-8-oxa-3-azabicyclo[3.2.1]octane (115 mg, 0.5 mmol) and 3-methylbutan-1-amine (78 mg, 1 mmol) afforded the title compound (34.8 mg, 10.2%) as a white solid. ¹H NMR (400 MHz, CDCl₃) δ 8.31 (s, 1H), 7.28-7.24 (m, 1H), 7.20 (d, J=8.4 Hz, 1H), 6.60-6.62 (d, J=8.0 Hz, 1H), 5.70 (bs, 1H), 4.49 (s, 2H), 4.05 (d, J=11.2 Hz, 2H), 3.53 (q, J=8.0 Hz, 2H), 3.15 (d, J=7.2 Hz, 2H), 2.21-2.18 (m, 2H), 2.03-1.99 (m, 2H), 1.75-1.69 (m, 1H), 1.61-1.57 (m, 1H), 0.98 (d, J=6.4 Hz, 6H). LC-MS m/z: 343.1 [M+H]⁺. HPLC Purity (214 nm): 99.0%; t_{R}=9.23 min.

### Example 222—4-(4-Acetylpiperazin-1-yl)-N-((1-iso-butyl-cyclopropyl)methyl)-1H-benzo[d]imidazole-1-carboxamide

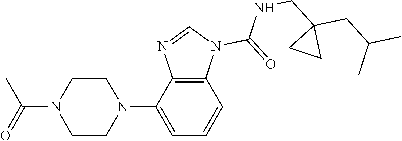 [SMILES: CC(=O)N1CCN(c2cccc3c2ncn3C(=O)NCC2(CC(C)C)CC2)CC1]

Following general procedure B, 1-(4-(1H-benzo[d]imidazol-4-yl)-piperazin-1-yl)ethanone (100 mg, 0.4 mmol) and (1-isobutylcyclopropyl)methanamine (50 mg, 0.4 mmol) afforded the title compound (68.8 mg, 25.6%) as a white solid. ¹H NMR (400 MHz, CDCl₃) δ 8.41 (s, 1H), 7.35 (d, J=4.5 Hz, 2H), 6.77 (t, J=4.4 Hz, 1H), 5.85 (bs, 1H), 3.90 (t, J=4.8 Hz, 2H), 3.76 (t, J=5.1 Hz, 2H), 3.59 (t, J=4.8 Hz, 2H), 3.47 (t, J=5.0 Hz, 2H), 3.43 (d, J=5.4 Hz, 2H), 2.18 (s, 3H), 1.94 (sept, J=6.9 Hz, 1H), 1.32 (d, J=7.4 Hz, 2H), 0.98 (d, J=6.6 Hz, 6H), 0.63-0.43 (m, 4H). LC-MS m/z: 398.0 [M+H]⁺. HPLC Purity (214 nm): 100%; t_{R}=8.68 min.

### Example 223—4-(4-Acetylpiperazin-1-yl)-N-(5-methylhexyl)-1H-benzo[d]imidazole-1-carboxamide

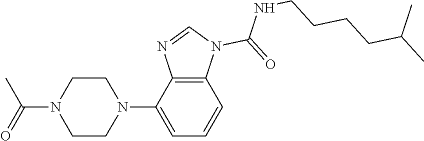 [SMILES: CC(=O)N1CCN(c2cccc3c2ncn3C(=O)NCCCCC(C)C)CC1]

Following general procedure C, 1-(4-(1H-benzo[d]imidazol-4-yl)piperazin-1-yl)ethan-1-one (200 mg, 0.82 mmol) and 5-methylhexan-1-amine (124 mg, 1 mmol) afforded the title compound (39.2 mg, 12.4%) as a white solid. ¹H NMR (400 MHz, CDCl₃) δ 8.37 (s, 1H), 7.39 (d, J=7.8 Hz, 1H), 7.30 (t, J=8.1 Hz, 1H), 6.73 (d, J=7.7 Hz, 1H), 6.02 (t, J=5.4 Hz, 1H), 3.90-3.87 (m, 2H), 3.76-3.62 (m, 2H), 3.58-3.49 (m, 4H), 3.44 (t, J=5.2 Hz, 2H), 2.16 (s, 3H), 1.70 (dd, J=14.9, 7.5 Hz, 2H), 1.56 (sept, J=6.7 Hz, 1H), 1.42 (ddd, J=11.9, 11.1, 7.4 Hz, 2H), 1.25 (dd, J=15.7, 6.9 Hz, 2H), 0.89 (d, J=6.6 Hz, 6H). LC-MS m/z: 386.1 [M+H]⁺. HPLC Purity (214 nm): 95.64%; t_{R}=8.70 min.

### Examples 224a and 224b—N-(3-(2,4-Difluorophenyl)propyl)-2-methoxy-6-morpholino-1H-benzo[d]imidazole-1-carboxamide and N-(3-(2,4-difluorophenyl)propyl)-2-methoxy-5-morpholino-1H-benzo[d]-imidazole-1-carboxamide

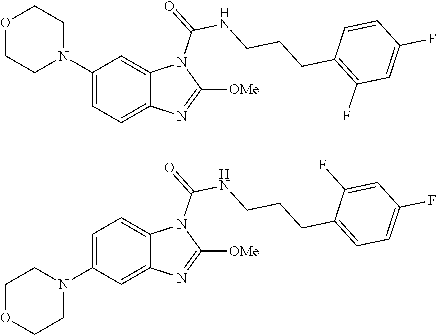 [SMILES: COc1nc2ccc(N3CCOCC3)cc2n1C(=O)NCCCc1ccc(F)cc1F ; COc1nc2cc(N3CCOCC3)ccc2n1C(=O)NCCCc1ccc(F)cc1F]

Following general procedure C, 4-(2-methoxy-1H-benzo[d]imidazol-6-yl)morpholine (200 mg, 0.86 mmol) and 3-(2,4-difluorophenyl)propan-1-amine (176 mg, 1.03 mmol) afforded N-(3-(2,4-difluorophenyl)propyl)-2-methoxy-6-morpholino-1H-benzo[d]imidazole-1-carboxamide (102 mg, 18.4%) and N-(3-(2,4-difluorophenyl)propyl)-2-methoxy-5-morpholino-1H-benzo[d]-imidazole-1-carboxamide (44 mg, 11.9%) both as white solids.

N-(3-(2,4-difluorophenyl)propyl)-2-methoxy-6-morpholino-1H-benzo[d]imidazole-1-carboxamide: ¹H NMR (400 MHz, CDCl₃) δ 7.82 (d, J=2.2 Hz, 1H), 7.40 (d, J=8.7 Hz, 1H), 7.17 (dd, J=15.1, 8.2 Hz, 1H), 7.10 (b, 1H), 6.91 (dd, J=8.7, 2.2 Hz, 1H), 6.85-6.76 (m, 2H), 4.33 (s, 3H), 3.89 (t, J=4.4 Hz, 4H), 3.44 (dd, J=13.0, 6.7 Hz, 2H), 3.19 (t, J=4.8 Hz, 4H), 2.74 (t, J=7.2 Hz, 2H), 1.97 (p, J=7.2 Hz, 2H). LC-MS m/z: 431.0 [M+H]⁺. HPLC Purity (214 nm): 100%; t_{R}=7.50 min.

N-(3-(2,4-difluorophenyl)propyl)-2-methoxy-5-morpholino-1H-benzo[d]-imidazole-1-carboxamide: ¹H NMR (400 MHz, CDCl₃) δ 8.03 (d, J=8.9 Hz, 1H), 7.18 (dd, J=15.0, 8.5 Hz, 1H), 7.07 (d, J=2.3 Hz, 1H), 7.03 (t, J=5.5 Hz, 1H), 6.93-6.86 (m, 1H), 6.86-6.75 (m, 2H), 4.32 (s, 3H), 3.91 (t, J=4.4 Hz, 4H), 3.44 (dd, J=12.8, 6.7 Hz, 2H), 3.16 (t, J=4.8 Hz, 4H), 2.72 (t, J=7.5 Hz, 2H), 2.97 (p, J=7.5 Hz, 2H). LC-MS m/z: 431.0 [M+H]⁺. HPLC Purity (214 nm): 100%; t_{R}=9.75 min.

### Example 225—4-(4-Acetylpiperazin-1-yl)-N-(4,4,4-trifluorobutyl)-1H-benzo[d]imidazole-1-carboxamide

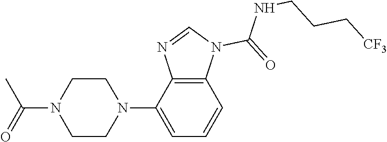 [SMILES: CC(=O)N1CCN(c2cccc3c2ncn3C(=O)NCCCC(F)(F)F)CC1]

Following general procedure B, 1-(4-(1H-benzo[d]imidazol-4-yl)-piperazin-1-yl)ethanone (200 mg, 0.82 mmol) and 4,4,4-trifluorobutan-1-amine (104 mg, 0.82 mmol) afforded the title compound (129.5 mg, 39.8%) as a white solid. ¹H NMR (400 MHz, CDCl₃) δ 8.41 (s, 1H), 7.46 (d, J=7.8 Hz, 1H), 7.32 (t, J=8.1 Hz, 1H), 6.74 (d, J=7.6 Hz, 1H), 6.60 (bs, 1H), 3.83 (t, J=4.4 Hz, 2H), 3.73 (t, J=4.8 Hz, 2H), 3.62 (dd, J=13.2, 6.8 Hz, 2H), 3.52 (t, J=5.2 Hz, 2H), 3.45 (t, J=5.2 Hz, 2H), 2.33-2.20 (m, 2H), 2.17 (s, 3H), 2.04 (p, J=7.5 Hz, 2H). LC-MS m/z: 398.0 [M+H]⁺. HPLC Purity (254 nm): 100%; t_{R}=7.44 min.

### Example 226—4-((2R,6S)-2,6-dimethylmorpholino)-N-(4,4,4-trifluorobutyl)-1H-benzo[d]imidazole-1-carboxamide

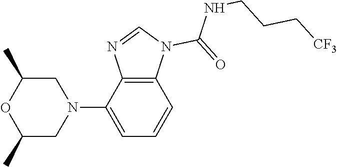 [SMILES: C[C@@H]1CN(c2cccc3c2ncn3C(=O)NCCCC(F)(F)F)C[C@H](C)O1]

Following general procedure E, 3-fluoro-2-nitrobenzenamine (400.0 mg, 2.6 mmol) and (2S,6R)-2,6-dimethylmorpholine (249.2 mg, 2.2 mmol) afforded 3-((2S,6R)-2,6-dimethylmorpholino)-2-nitrobenzenamine (300.0 mg, 54.3%) as a yellow solid. LC-MS m/z: 252.2 [M+H]⁺. Purity (254 nm): 100.0%; t_{R}=1.85 min.

Following general procedure F, 3-((2S,6R)-2,6-dimethylmorpholino)-2-nitrobenzenamine (300.0 mg, 1.2 mmol) afforded 3-((2S,6R)-2,6-dimethylmorpholino)benzene-1,2-diamine (220.0 mg, 82.6%) as a brown oil which was used directly in the next step. LC-MS m/z: 222.2 [M+H]⁺. Purity (214 nm): 100.0%; t_{R}=1.64 min.

Following general procedure G (method A), 3-((2S,6R)-2,6-dimethylmorpholino) benzene-1,2-diamine (200.0 mg, 0.9 mmol) afforded (2S,6R)-4-(1H-benzo[d]imidazol-4-yl)-2,6-dimethylmorpholine (230.0 mg, 99.3%) as a yellow oil. LC-MS m/z: 232.2 [M+H]⁺. Purity (214 nm): 95.2%; t_{R}=1.58 min.

Following general procedure C, (2S,6R)-4-(1H-benzo[d]imidazol-4-yl)-2,6-dimethyl morpholine (230 mg, 0.9 mmol) and 4,4,4-trifluorobutan-1-amine (139.7 mg, 1.1 mmol) afforded the title compound (33.0 mg, 8.8%) as a white solid. ¹H NMR (400 MHz, CDCl₃) δ 8.36 (s, 1H), 7.29 (d, J=4.0 Hz, 2H), 6.74 (t, J=4.5 Hz, 1H), 5.98 (bs, 1H), 4.09-3.97 (m, 4H), 3.60 (dd, J=13.4, 6.9 Hz, 2H), 2.56 (t, J=9.7 Hz, 2H), 2.35-2.16 (m, 2H), 2.04-1.95 (m, 2H), 1.27 (d, J=6.0 Hz, 6H). LC-MS m/z: 385.2 [M+H]⁺. HPLC Purity (214 nm): 91.97%; t_{R}=8.31 min.

### Example 227—N-((1-iso-Butylcyclopropyl)methyl)-4-morpholino-1H-benzo[d]imidazole-1-carboxamide

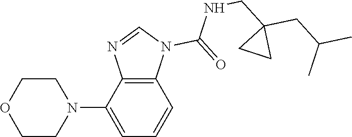 [SMILES: CC(C)CC1(CNC(=O)n2cnc3c(N4CCOCC4)cccc32)CC1]

To a solution of cyclopropanecarbonitrile (2.0 g, 30 mmol) in THF (10 mL) was added LDA (18 mL, 36 mmol) at −80° C. and the reaction mixture was stirred at −80° C. for 1.5 h. Then 1-iodo-2-methylpropane (11 g, 60 mmol) was added and the resulting mixture was stirred at RT overnight. The reaction mixture was quenched by the addition of saturated aq. NH₄Cl. The organic layer was dried over Na₂SO₄, filtered and concentrated in vacuo to give 1-iso-butylcyclopropane-1-carbonitrile (4.4 g, 100%6) as a yellow solid.

To a solution of 1-iso-butylcyclopropane-1-carbonitrile (4.4 g, 30 mmol) in THF (10 mL) was added AlCl₃ (4 g, 30 mmol) and LAH/THF (60 ml, 60 mmol). The reaction mixture was stirred at RT for 2 h. And then, the reaction mixture was quenched with Na₂SO₄.10H₂O. The organic layer was dried over Na₂SO₄, filtered and concentrated in vacuo to give (1-iso-butylcyclopropyl)methanamine (2.3 g, 10%) as a yellow solid. LC-MS m/z: 128.4 [M+H]⁺, t_{R}=0.84 min.

Following general procedure C, 1,4-(1H-benzo[d]imidazol-4-yl)morpholine (100 mg, 0.49 mmol) and (1-iso-butylcyclopropyl)methanamine (80 mg, 0.49 mmol) afforded the title compound (53.3 mg, 30.3%) as a white solid. ¹H NMR (400 MHz, CDCl₃) δ 8.37 (s, 1H), 7.37-7.27 (m, 2H), 6.75 (d, J=7.0 Hz, 1H), 5.77 (bs, 1H), 4.02-3.98 (m, 4H), 3.57-3.40 (m, 4H), 3.42 (d, J=5.4 Hz, 2H), 1.91 (septet, J=6.8 Hz, 1H), 1.29 (d, J=7.4 Hz, 2H), 0.96 (d, J=6.6 Hz, 6H), 0.57-0.53 (m, 2H), 0.50-0.46 (m, 2H). LC-MS m/z: 357.2 [M+H]⁺. HPLC Purity (214 nm): 100%; t_{R}=9.03 min.

### Examples 228a and 228b—N-(3-(Furan-2-yl)propyl)-2-methoxy-6-morpholino-1H-benzo[d]imidazole-1-carboxamide and N-(3-(furan-2-yl)propyl)-2-methoxy-5-morpholino-1H-benzo[d]imidazole-1-carboxamide

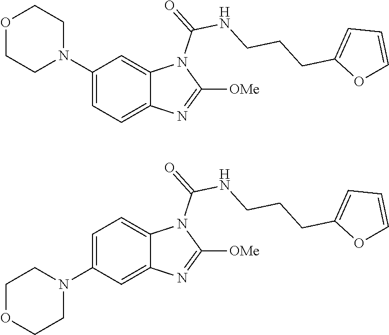 [SMILES: COc1nc2ccc(N3CCOCC3)cc2n1C(=O)NCCCc1ccco1 ; COc1nc2cc(N3CCOCC3)ccc2n1C(=O)NCCCc1ccco1]

Following general procedure B, 4-(2-methoxy-1H-benzo[d]imidazol-6-yl)morpholine (80 mg, 0.3 mmol) and 3-(furan-2-yl)propan-1-amine (50 mg, 0.4 mmol) afforded N-(3-(furan-2-yl)propyl)-2-methoxy-6-morpholino-1H-benzo[d]imidazole-1-carboxamide (29.1 mg 18.9%) and N-(3-(furan-2-yl)propyl)-2-methoxy-5-morpholino-1H-benzo[d]imidazole-1-carboxamide (23.1 mg 15.0%) both as white solids.

N-(3-(furan-2-yl)propyl)-2-methoxy-6-morpholino-1H-benzo[d]imidazole-1-carboxamide: ¹H NMR (400 MHz, CDCl₃) δ 7.82 (d, J=2.4 Hz, 1H), 7.38 (d, J=9.6 Hz, 1H), 7.30 (d, J=1.2 Hz, 1H), 7.05 (t, J=5.5 Hz, 1H), 6.90 (dd, J=8.7, 2.4 Hz, 1H), 6.30 (dd, J=3.6, 2.0 Hz, 1H), 6.02 (d, J=2.8 Hz, 1H), 4.30 (s, 3H), 3.94-3.75 (m, 4H), 3.47 (dd, J=13.0, 6.8 Hz, 2H), 3.24-3.03 (m, 4H), 2.75 (t, J=7.3 Hz, 2H), 2.01 (p, J=7.2 Hz, 2H). LC-MS m/z: 384.7 [M+H]⁺. HPLC Purity (214 nm): 100%; t_{R}=3.93 min.

N-(3-(furan-2-yl)propyl)-2-methoxy-5-morpholino-1H-benzo[d]imidazole-1-carboxamide: ¹H NMR (400 MHz, CDCl₃) δ 8.03 (d, J=8.9 Hz, 1H), 7.31 (s, 1H), 7.06 (d, J=2.1 Hz, 1H), 6.96 (bs, 1H), 6.88 (dd, J=8.9, 2.3 Hz, 1H), 6.31 (t, J=2.0 Hz, 1H), 6.04 (d, J=2.7 Hz, 1H), 4.30 (s, 3H), 3.92-3.87 (m, 4H), 3.47 (dd, J=13.0, 6.7 Hz, 2H), 3.18-3.14 (m, 4H), 2.75 (t, J=7.3 Hz, 2H), 2.00 (p, J=7.1 Hz, 2H). LC-MS m/z: 384.7 [M+H]⁺. HPLC Purity (214 nm): 100%; t_{R}=3.97 min.

### Examples 229a and 229b—N-((1-(Cyclopropylmethyl)cyclopropyl)-methyl)-2-methoxy-6-morpholino-1H-benzo[d]imidazole-1-carboxamide and N-((1-(cyclopropylmethyl) cyclopropyl)methyl)-2-methoxy-5-morpholino-1H-benzo[d]imidazole-1-carboxamide

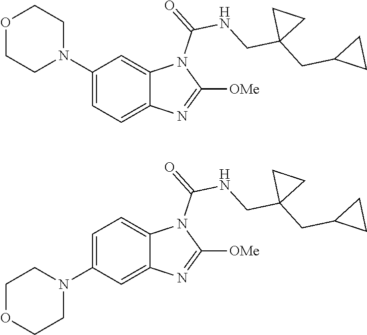 [SMILES: COc1nc2ccc(N3CCOCC3)cc2n1C(=O)NCC1(CC2CC2)CC1 ; COc1nc2cc(N3CCOCC3)ccc2n1C(=O)NCC1(CC2CC2)CC1]

Following general procedure C, 4-(2-methoxy-1H-benzo[d]imidazol-6-yl)morpholine (94 mg, 0.4 mmol) and 3-(1-(trifluoromethyl)cyclopropyl)propan-1-amine (35 mg, 0.4 mmol) afforded N-((1-(cyclopropylmethyl)cyclopropyl)-methyl)-2-methoxy-6-morpholino-1H-benzo[d]imidazole-1-carboxamide (21.4 mg, 14.1%) and N-((1-(cyclopropylmethyl)cyclopropyl) methyl)-2-methoxy-5-morpholino-1H-benzo[d]imidazole-1-carboxamide (18.9 mg, 12.3%) both as white solids.

N-((1-(cyclopropylmethyl)cyclopropyl)-methyl)-2-methoxy-6-morpholino-1H-benzo[d]imidazole-1-carboxamide: ¹H NMR (400 MHz, CDCl₃) δ 8.03 (d, J=8.9 Hz, 1H), 7.07 (d, J=2.4 Hz, 1H), 7.02 (bs, 1H), 6.89 (dd, J=8.9, 2.4 Hz, 1H), 4.31 (s, 3H), 4.04-3.75 (m, 4H), 3.45 (d, J=5.4 Hz, 2H), 3.28-3.01 (m, 4H), 1.31 (d, J=6.8 Hz, 2H), 0.76 (td, J=11.7, 5.7 Hz, 1H), 0.63-0.48 (m, 6H), 0.07 (q, J=5.0 Hz, 2H). LC-MS m/z: 385.2 [M+H]⁺. HPLC Purity (214 nm): 100%; t_{R}=8.17 min.

N-((1-(cyclopropylmethyl)cyclopropyl) methyl)-2-methoxy-5-morpholino-1H-benzo[d]imidazole-1-carboxamide: ¹H NMR (400 MHz, CDCl₃) δ 7.84 (d, J=2.4 Hz, 1H), 7.41 (d, J=8.8 Hz, 1H), 7.12 (bs, 1H), 6.92 (dd, J=8.9, 2.4 Hz, 1H), 4.31 (s, 3H), 3.91-3.87 (m, 4H), 3.45 (d, J=5.4 Hz, 2H), 3.21-3.17 (m, 4H), 1.31 (d, J=6.8 Hz, 2H), 0.76 (td, J=11.7, 5.7 Hz, 1H), 0.60-0.33 (m, 6H), 0.07 (q, J=5.0 Hz, 2H). LC-MS m/z: 385.2 [M+H]⁺. HPLC Purity (214 nm): 100%; t_{R}=8.15 min.

### Examples 230a and 230b—2-Methoxy-6-morpholino-N-(2-(2-(trifluoromethyl)cyclo propyl)ethyl)-1H-benzo[d]imidazole-1-carboxamide and 2-methoxy-5-morpholino-N-(2-(2-(trifluoromethyl)cyclopropyl)-ethyl)-1H-benzo[d]imidazole-1-carboxamide

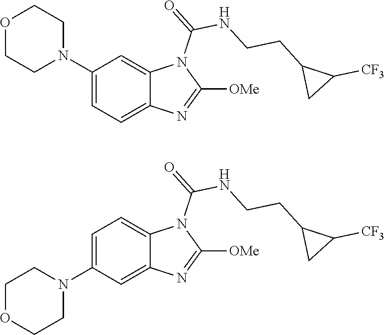 [SMILES: COc1nc2ccc(N3CCOCC3)cc2n1C(=O)NCCC1CC1C(F)(F)F ; COc1nc2cc(N3CCOCC3)ccc2n1C(=O)NCCC1CC1C(F)(F)F]

Following general procedure C, 4-(2-methoxy-1H-benzo[d]imidazol-6-yl)morpholine (160 mg, 0.64 mmol) and 2-(2-(trifluoromethyl)cyclopropyl)ethan-1-amine (300 mg, 2 mmol) afforded 2-methoxy-6-morpholino-N-(2-(2-(trifluoromethyl)cyclopropyl)ethyl)-1H-benzo[d]imidazole-1-carboxamide (11.4 mg, 5.4%) and 2-methoxy-5-morpholino-N-(2-(2-(trifluoromethyl) cyclopropyl)-ethyl)-1H-benzo[d]imidazole-1-carboxamide (9.4 mg, 4.3%) both as white solids.

2-methoxy-6-morpholino-N-(2-(2-(trifluoromethyl)cyclopropyl)ethyl)-1H-benzo[d]imidazole-1-carboxamide: ¹H NMR (400 MHz, CDCl₃) δ 8.02 (d, J=8.9 Hz, 1H), 7.11-7.07 (m, 2H), 6.89 (dd, J=8.9, 2.4 Hz, 1H), 4.33 (s, 3H), 3.94-3.88 (m, 4H), 3.64-3.52 (m, 2H), 3.17 (t, J=4.8 Hz, 4H), 1.76 (dt, J=13.8, 7.0 Hz, 1H), 1.64-1.54 (m, 1H), 1.43-1.37 (m, 1H), 1.28-1.19 (m, 1H), 1.10-1.01 (m, 1H), 0.71 (dt, J=14.3, 6.0 Hz, 1H). LC-MS m/z: 413.1 [M+H]⁺. HPLC Purity (214 nm): =100%; t_{R}=7.21 min.

2-methoxy-5-morpholino-N-(2-(2-(trifluoromethyl)cyclopropyl)-ethyl)-1H-benzo[d]imidazole-1-carboxamide: ¹H NMR (400 MHz, CDCl₃) δ 7.82 (d, J=5.2 Hz, 1H), 7.40 (d, J=8.7 Hz, 1H), 7.19 (t, J=5.2 Hz, 1H), 6.90 (dd, J=8.9, 2.8 Hz, 1H), 4.31 (s, 3H), 3.92-3.88 (m, 4H), 3.53 (dt, J=13.7, 6.7 Hz, 2H), 3.20-3.16 (m, 4H), 1.77 (dt, J=13.6, 6.9 Hz, 1H), 1.65-1.55 (m, 1H), 1.44-1.37 (m, 1H), 1.27-1.18 (m, 1H), 1.10-1.01 (m, 1H), 0.75-0.67 (m, 1H). LC-MS m/z: 413.1 [M+H]⁺. HPLC Purity (214 nm): =100%; t_{R}=7.16 min.

### Examples 231a and 231b—N-((2-benzylcyclopropyl)methyl)-2-methoxy-6-morpholino-1H-benzo[d]imidazole-1-carboxamide and N-((2-benzylcyclopropyl)methyl)-2-methoxy-5-morpholino-1H-benzo[d]imidazole-1-carboxamide

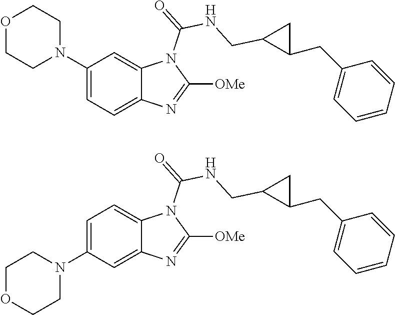 [SMILES: COc1nc2ccc(N3CCOCC3)cc2n1C(=O)NCC1CC1Cc1ccccc1 ; COc1nc2cc(N3CCOCC3)ccc2n1C(=O)NCC1CC1Cc1ccccc1]

Following general procedure C, 4-(2-methoxy-1H-benzo[d]imidazol-6-yl)morpholine (100 mg, 0.43 mmol) and (2-benzylcyclopropyl)methanamine (69.1 mg, 0.43 mmol) afforded N-((2-benzylcyclopropyl)methyl)-2-methoxy-6-morpholino-1H-benzo[d]imidazole-1-carboxamide (35.7 mg, 19.8%) and N-((2-benzylcyclopropyl)methyl)-2-methoxy-5-morpholino-1H-benzo[d]imidazole-1-carboxamide (33.8 mg, 18.7%) both as yellow solids.

N-((2-benzylcyclopropyl)methyl)-2-methoxy-6-morpholino-1H-benzo[d]imidazole-1-carboxamide: 1H NMR (400 MHz, CDCl₃) δ 7.82 (d, J=2.4 Hz, 1H), 7.40 (d, J=8.7 Hz, 1H), 7.22-7.11 (m, 5H), 6.96-6.88 (m, 2H), 4.19 (s, 3H), 3.87 (t, J=4.8 Hz, 4H), 3.42 (dd, J=13.0, 6.9 Hz, 1H), 3.19-3.18 (m, 5H), 2.76 (dd, J=14.6, 5.8 Hz, 1H), 2.42 (dd, J=14.6, 7.5 Hz, 1H), 1.04 (dd, J=13.5, 6.2 Hz, 2H), 0.62-0.51 (m, 2H). LC-MS m/z: 420.7 [M+H]⁺. HPLC Purity (214 nm): 97.14%; t_{R}=8.61 min.

N-((2-benzylcyclopropyl)methyl)-2-methoxy-5-morpholino-1H-benzo[d]imidazole-1-carboxamide: 1H NMR (400 MHz, CDCl₃) δ 8.02 (d, J=8.9 Hz, 1H), 7.22-7.11 (m, 5H), 7.07 (d, J=2.3 Hz, 1H), 6.91-6.84 (m, 2H), 4.19 (s, 3H), 3.92-3.88 (m, 4H), 3.45-3.41 (m, 1H), 3.20-3.14 (m, 4H), 2.76 (dd, J=14.4, 5.6 Hz, 1H), 2.42 (dd, J=14.8, 7.6 Hz, 1H), 1.25 (s, 1H), 1.03 (d, J=6.0 Hz, 2H), 0.61-0.54 (m, 2H). LC-MS m/z: 420.7 [M+H]⁺. HPLC Purity (214 nm): 97.18%; t_{R}=8.12 min.

### Examples 232a and 232b—N-(3-(6-Fluoropyridin-3-yl)propyl)-2-methoxy-6-morpholino-1H-benzo[d]imidazole-1-carboxamide and N-(3-(6-fluoropyridin-3-yl)propyl)-2-methoxy-5-morpholino-1H-benzo[d]imidazole-1-carboxamide

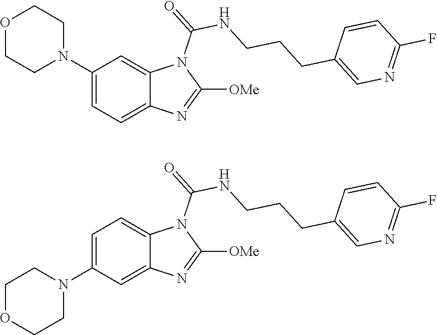 [SMILES: COc1nc2ccc(N3CCOCC3)cc2n1C(=O)NCCCc1ccc(F)nc1 ; COc1nc2cc(N3CCOCC3)ccc2n1C(=O)NCCCc1ccc(F)nc1]

Following general procedure C, 4-(2-methoxy-1H-benzo[d]imidazol-6-yl)morpholine (100 mg, 0.43 mmol) and 3-(6-fluoropyridin-3-yl)propan-1-amine (79 mg, 0.51 mmol) afforded N-(3-(6-fluoropyridin-3-yl)propyl)-2-methoxy-6-morpholino-1H-benzo[d]imidazole-1-carboxamide (11.3 mg, 8%) and N-(3-(6-fluoropyridin-3-yl)propyl)-2-methoxy-5-morpholino-1H-benzo[d]imidazole-1-carboxamide (7.7 mg, 6%) both as white solids.

N-(3-(6-fluoropyridin-3-yl)propyl)-2-methoxy-6-morpholino-1H-benzo[d]imidazole-1-carboxamide: ¹H NMR (400 MHz, CDCl₃) δ 8.09 (d, J=1.6 Hz, 1H), 7.83 (d, J=2.4 Hz, 1H), 7.66 (dt, J=8.8, 2.4 Hz, 1H), 7.42 (d, J=8.8 Hz, 1H), 7.07 (t, J=5.2 Hz, 1H), 6.94-6.88 (m, 1H), 4.31 (s, 3H), 3.89 (t, J=4.8 Hz, 4H), 3.50 (q, J=6.8 Hz, 2H), 3.19 (t, J=4.8 Hz, 4H), 2.74 (t, J=7.6 Hz, 2H), 2.01 (p, J=7.2 Hz, 2H). LC-MS m/z: 414.2 [M+H]⁺. HPLC Purity (214 nm): 100%; t_{R}=6.81 min.

N-(3-(6-fluoropyridin-3-yl)propyl)-2-methoxy-5-morpholino-1H-benzo[d]imidazole-1-carboxamide: ¹H NMR (400 MHz, CDCl₃) δ 8.09 (d, J=2.0 Hz, 1H), 8.05 (d, J=8.8 Hz, 1H), 7.67 (dt, J=8.8, 2.4 Hz, 1H), 7.09 (d, J=2.4 Hz, 1H), 7.00-6.97 (t, J=5.2 Hz, 1H), 6.90 (dt, J=8.0, 2.8 Hz, 2H), 4.33 (s, 3H), 3.91 (t, J=4.8 Hz, 4H), 3.50 (q, J=6.8 Hz, 2H), 3.17 (t, J=4.8 Hz, 4H), 2.74 (t, J=7.6 Hz, 2H), 2.05 (p, J=7.2 Hz, 2H). LC-MS m/z: 414.2 [M+H]⁺. HPLC Purity (214 nm): 98.67%; t_{R}=6.84 min.

### Examples 233a and 233b—2-Methoxy-6-morpholino-N-(3-(trifluoromethoxy)propyl)-1H-benzo[d]imidazole-1-carboxamide and 2-methoxy-5-morpholino-N-(3-(trifluoromethoxy) propyl)-1H-benzo[d]imidazole-1-carboxamide

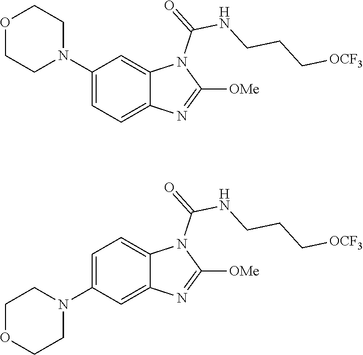 [SMILES: COc1nc2ccc(N3CCOCC3)cc2n1C(=O)NCCCOC(F)(F)F ; COc1nc2cc(N3CCOCC3)ccc2n1C(=O)NCCCOC(F)(F)F]

Following general procedure C, 4-(2-methoxy-1H-benzo[d]imidazol-5-yl)morpholine (100 mg, 0.43 mmol) and 3-(trifluoromethoxy)propan-1-amine (100 mg, 0.56 mmol) afforded 2-methoxy-6-morpholino-N-(3-(trifluoromethoxy)propyl)-1H-benzo[d]imidazole-1-carboxamide (33.5 mg, 19.5%) and 2-methoxy-5-morpholino-N-(3-(trifluoromethoxy)propyl)-1H-benzo[d]imidazole-1-carboxamide (32.2 mg, 18.6%) both as white solids.

2-methoxy-6-morpholino-N-(3-(trifluoromethoxy)propyl)-1H-benzo[d]imidazole-1-carboxamide: ¹H NMR (400 MHz, CDCl₃) δ 7.80 (d, J=2.4 Hz, 1H), 7.39 (d, J=8.4 Hz, 1H), 7.21 (t, J=5.2 Hz, 1H), 6.91 (dd, J=8.8 Hz, 2.4 Hz, 1H), 4.29 (s, 3H), 4.13 (t, J=6.0 Hz, 2H), 3.87 (t, J=4.8 Hz, 4H), 3.59 (q, J=6.4 Hz, 2H), 3.17 (t, J=4.8 Hz, 4H), 2.07 (p, J=6.0 Hz, 2H). LC-MS m/z: 403.0 [M+H]⁺. HPLC Purity (214 nm): 100%; t_{R}=7.57 min.

2-methoxy-5-morpholino-N-(3-(trifluoromethoxy)propyl)-1H-benzo[d]imidazole-1-carboxamide: ¹H NMR (400 MHz, CDCl₃) δ 8.01 (d, J=8.8 Hz, 1H), 7.13 (t, J=5.6 Hz, 1H), 7.07 (s, 1H), 6.88 (d, J=8.8 Hz, 1H), 4.30 (s, 3H), 4.12 (t, J=6.0 Hz, 2H), 3.87 (bs, 4H), 3.59 (q, J=6.0 Hz, 2H), 3.16 (bs, 4H), 2.06 (p, J=6.0 Hz, 2H). LC-MS m/z: 403.0 [M+H]⁺. HPLC Purity (214 nm): 100%; t_{R}=7.55 min.

### Example 234—4-(4-iso-Butyrylpiperazin-1-yl)-N-(3-(1-(trifluoromethyl)cyclopropyl) propyl)-1H-benzo[d]imidazole-1-carboxamide

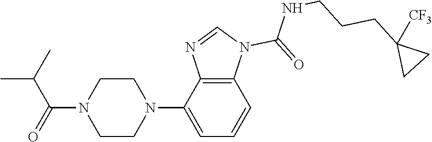 [SMILES: CC(C)C(=O)N1CCN(c2cccc3c2ncn3C(=O)NCCCC2(C(F)(F)F)CC2)CC1]

Following general procedure E, 3-fluoro-2-nitroaniline (1.0 g, 6.41 mmol) and 2-methyl-1-(piperazin-1-yl)propan-1-one hydrochloride (1.5 g, 9.62 mmol) afforded 1-(4-(3-amino-2-nitrophenyl)piperazin-1-yl)-2-methylpropan-1-one (1.3 g, 70%) as an orange solid. LC-MS m/z: 293.0 [M+H]⁺. Purity (214 nm): 99%; t_{R}=1.80 min.

Following general procedure F, 1-(4-(3-amino-2-nitrophenyl)piperazin-1-yl)-2-methylpropan-1-one (1.3 g, 4.45 mmol) afforded 1-(4-(2,3-diaminophenyl)piperazin-1-yl)-2-methylpropan-1-one (1.1 g, 95%) as a brown solid which was used directly in the next step. LC-MS m/z: 263.1 [M+H]⁺. Purity (214 nm): 99%; t_{R}=1.38 min.

Following general procedure G (method A), 1-(4-(2,3-diaminophenyl)piperazin-1-yl)-2-methylpropan-1-one (400 mg, 1.53 mmol) afforded 1-(4-(1H-benzo[d]imidazol-4-yl)piperazin-1-yl)-2-methylpropan-1-one (310 mg, 75%) as a clear oil. LC-MS m/z: 273.2 [M+H]⁺. Purity (214 nm): 87%; t_{R}=1.44 min.

Following general procedure C, 1-(4-(1H-benzo[d]imidazol-4-yl)piperazin-1-yl)-2-methylpropan-1-one (85 mg, 0.31 mmol) and 3-(1-(trifluoromethyl)cyclopropyl)propan-1-amine (105 mg, 0.63 mmol) afforded the title compound (17.6 mg, 12%) as a white solid. ¹H NMR (400 MHz, CDCl₃) δ 8.36 (s, 1H), 7.33 (dd, J=18.4, 7.7 Hz, 2H), 6.73 (d, J=7.8 Hz, 1H), 5.98 (t, J=5.4 Hz, 1H), 3.91-3.84 (m, 2H), 3.81-3.74 (m, 2H), 3.60-3.54 (m, 2H), 3.51 (dd, J=13.2, 7.2 Hz, 2H), 3.47-3.41 (m, 2H), 2.86 (sept, J=6.8 Hz, 1H), 1.94-1.81 (m, 2H), 1.73-1.65 (m, 2H), 1.17 (d, J=6.8 Hz, 6H), 1.00 (t, J=5.9 Hz, 2H), 0.61 (bs, 2H). LC-MS m/z: 466.1 [M+H]⁺. Purity (214 nm): 99%; t_{R}=8.85 min.

### Example 235—4-(4-Acetylpiperazin-1-yl)-N-(3-(1-(trifluoromethyl)cyclopropyl)propyl)-1H-benzo[d]imidazole-1-carboxamide

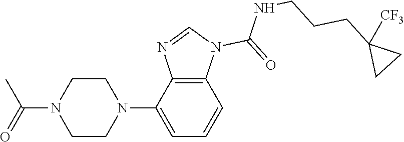 [SMILES: CC(=O)N1CCN(c2cccc3c2ncn3C(=O)NCCCC2(C(F)(F)F)CC2)CC1]

Following general procedure C, 1-(4-(1H-benzo[d]imidazol-4-yl)piperazin-1-yl)ethan-1-one (70 mg, 0.4 mmol) and 3-(1-(trifluoromethyl)-cyclopropyl)propan-1-amine (44 mg, 0.4 mmol) afforded the title compound (17.5 mg, 35.6%) as a white solid. ¹H NMR (400 MHz, CDCl₃) δ 8.37 (s, 1H), 7.39 (d, J=7.9 Hz, 1H), 7.30 (t, J=8.1 Hz, 1H), 6.72 (d, J=7.7 Hz, 1H), 6.19 (bs, 1H), 3.87 (t, J=4.4 Hz, 2H), 3.87 (t, =4.4 Hz, 2H), 3.59-3.50 (m, 4H), 3.46 (t, =4.4 Hz, 2H), 2.15 (s, 3H), 1.88 (dt, J=15.6, 7.6 Hz, 2H), 1.73-1.66 (m, 2H), 1.08-0.89 (m, 2H), 0.61 (bs, 2H). LC-MS m/z: 438.1 [M+H]⁺. HPLC Purity (214 nm): 100%; t_{R}=8.29 min.

### Examples 236a and 236b—5-(4-Acetylpiperazin-1-yl)-2-methoxy-N-(3-phenylpropyl)-1H-benzo[d]imidazole-1-carboxamide and 6-(4-acetylpiperazin-1-yl)-2-methoxy-N-(3-phenyl propyl)-1H-benzo[d]imidazole-1-carboxamide

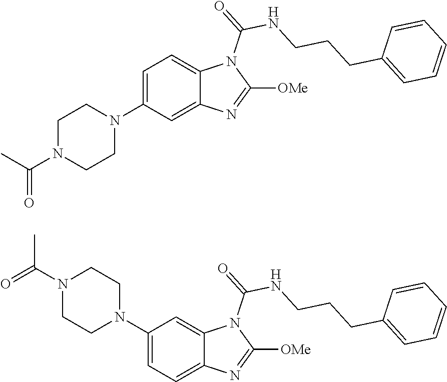

Following general procedure E, 5-fluoro-2-nitrobenzenamine (3.00 g, 19.23 mmol) and 1-(piperazin-1-yl)ethanone (4.93 g, 38.45 mmol) afforded 1-(4-(3-amino-4-nitrophenyl)piperazin-1-yl)ethanone (2.0 g, 40%) as a yellow solid. LC-MS m/z: 265.5 [M+H]⁺. HPLC Purity (214 nm): >⁹⁶%; t_{R}=1.47 min.

Following general procedure F, 1-(4-(3-amino-4-nitrophenyl)piperazin-1-yl)ethanone (2.00 g, 7.57 mmol) afforded 1-(4-(3,4-diaminophenyl)piperazin-1-yl)ethanone (1.2 g, 67.4%) as a yellow solid. LC-MS m/z: 235.2 [M+H]⁺. Purity (214 nm): >90%; t_{R}=0.57 min.

Following general procedure G (method A), 1-(4-(3,4-diaminophenyl)piperazin-1-yl)ethanone (1.20 g, 5.12 mmol) afforded 1-(4-(2-methoxy-3H-benzo[d]imidazol-5-yl)piperazin-1-yl)ethanone (0.5 g, 35.7%) as a yellow solid. LC-MS m/z: 275.2 [M+H]⁺. Purity (214 nm): 79%; t_{R}=1.27 min.

Following general procedure C, 1-(4-(2-methoxy-3H-benzo[d]imidazol-5-yl)piperazin-1-yl)ethanone (0.30 g, 1.09 mmol) and 3-phenylpropan-1-amine (0.16 g, 1.20 mmol) afforded 5-(4-acetylpiperazin-1-yl)-2-methoxy-N-(3-phenylpropyl)-1H-benzo[d]imidazole-1-carboxamide (70.7 mg, 14.8%) and 6-(4-acetylpiperazin-1-yl)-2-methoxy-N-(3-phenylpropyl)-1H-benzo[d]imidazole-1-carboxamide (83.1 mg, 17.4%) both as white solids.

5-(4-acetylpiperazin-1-yl)-2-methoxy-N-(3-phenylpropyl)-1H-benzo[d]imidazole-1-carboxamide: ¹H NMR (400 MHz, CDCl₃) δ 8.04 (d, J=8.8 Hz, 1H), 7.31-7.27 (m, 2H), 7.23-7.18 (m, 3H), 7.06 (d, J=2.4 Hz, 1H), 6.90 (dd, J=9.2, 2.4 Hz, 2H), 4.28 (s, 3H), 3.79 (t, J=4.8 Hz, 2H), 3.64 (t, J=5.2 Hz, 2H), 3.46 (dd, J=13.2 Hz, 7.2 Hz, 2H), 3.17-3.11 (m, 4H), 2.73 (t, J=7.6 Hz, 2H), 2.14 (s, 3H), 1.99 (p, J=6.8 Hz, 2H). LC-MS m/z: 436.2 [M+H]⁺. HPLC Purity (214 nm): 100%; t_{R}=7.90 min.

6-(4-acetylpiperazin-1-yl)-2-methoxy-N-(3-phenylpropyl)-1H-benzo[d]imidazole-1-carboxamide: ¹H NMR (400 MHz, CDCl₃) δ 7.84 (d, J=2.4 Hz, 1H), 7.39 (d, J=8.8 Hz, 1H), 7.31-7.26 (m, 2H), 7.23-7.18 (m, 3H), 6.97 (bs, 1H), 6.90 (dd, J=8.8, 2.4 Hz, 1H), 4.27 (s, 3H), 3.78 (t, J=4.8 Hz, 2H), 3.62 (t, J=5.2 Hz, 2H), 3.46 (dd, J=13.2 Hz, 7.2 Hz, 2H), 3.19-3.13 (m, 4H), 2.73 (t, J=7.6 Hz, 2H), 2.14 (s, 3H), 2.00 (p, J=7.6 Hz, 2H). LC-MS m/z: 436.2 [M+H]⁺. HPLC Purity (214 nm): 96.99%; t_{R}=7.33 min.

### Examples 237a and 237b—5-((1R,5S)-3-Oxa-8-azabicyclo[3.2.1]octan-8-yl)-2-methoxy-N-(3-phenylpropyl)-1H-benzo[d]imidazole-1-carboxamide and 6-((1R,5S)-3-oxa-8-azabicyclo[3.2.1]octan-8-yl)-2-methoxy-N-(3-phenylpropyl)-1H-benzo[d]imidazole-1-carboxamide

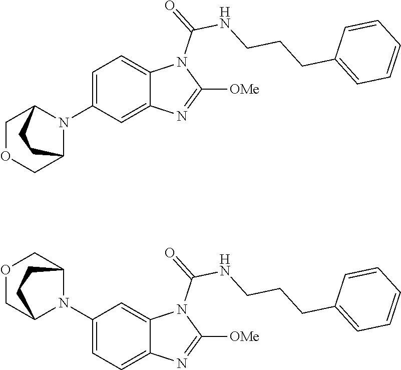

Following general procedure C, (1R,5S)-8-(2-methoxy-1H-benzo[d]imidazol-5-yl)-3-oxa-8-azabicyclo[3.2.1]octane (0.30 g, 1.16 mmol) and 3-phenylpropan-1-amine (0.17 g, 1.27 mmol) afforded 5-((1R,5S)-3-oxa-8-azabicyclo[3.2.1]octan-8-yl)-2-methoxy-N-(3-phenylpropyl)-1H-benzo[d]imidazole-1-carboxamide (61.5 mg, 12.6%) and 6-((1R,5S)-3-oxa-8-azabicyclo[3.2.1]octan-8-yl)-2-methoxy-N-(3-phenylpropyl)-1H-benzo[d]imidazole-1-carboxamide (170.8 mg, 35.1%) both as white solids.

5-((1R,5S)-3-oxa-8-azabicyclo[3.2.1]octan-8-yl)-2-methoxy-N-(3-phenylpropyl)-1H-benzo[d]imidazole-1-carboxamide: ¹H NMR (400 MHz, CDCl₃) δ 7.99 (d, J=8.8 Hz, 1H), 7.31-7.26 (m, 2H), 7.21-7.20 (m, 3H), 6.94 (d, J=2.4 Hz, 1H), 6.93-6.87 (m, 1H), 6.74 (dd, J=9.0, 2.4 Hz, 1H), 4.28 (s, 3H), 4.04 (bs, 2H), 3.96 (d, J=10.8 Hz, 2H), 3.53 (d, J=10.8 Hz, 2H), 3.45 (q, J=6.8 Hz, 2H), 2.73 (t, J=7.6 Hz, 2H), 2.07-1.97 (m, 6H). LC-MS m/z: 421.0 [M+H]⁺. HPLC Purity (214 nm): 100%; t_{R}=8.40 min.

6-((1R,5S)-3-oxa-8-azabicyclo[3.2.1]octan-8-yl)-2-methoxy-N-(3-phenylpropyl)-1H-benzo[d]imidazole-1-carboxamide: ¹H NMR (400 MHz, CDCl₃) δ 7.71 (d, J=2.4 Hz, 1H), 7.35 (d, J=8.8 Hz, 1H), 7.31-7.26 (m, 2H), 7.21-7.20 (m, 3H), 6.98 (m, 1H), 6.76 (dd, J=8.8, 2.4 Hz, 1H), 4.26 (s, 3H), 4.08 (bs, 2H), 3.96 (d, J=10.8 Hz, 2H), 3.54 (d, J=10.8 Hz, 2H), 3.45 (q, J=6.8 Hz, 2H), 2.73 (t, J=7.6 Hz, 2H), 2.06-1.97 (m, 6H). LC-MS m/z: 421.0 [M+H]⁺. HPLC Purity (214 nm): 100%; t_{R}=8.38 min.

### Examples 238a and 238b—2-Methoxy-N-(3-phenylpropyl)-6-(2-oxa-7-azaspiro[3,5]nonan-7-yl)-1H-benzo[d]imidazole-1-carboxamide and 2-methoxy-N-(3-phenylpropyl)-5-(2-oxa-7-azaspiro[3.5]nonan-7-yl)-1H-benzo[d]-imidazole-1-carboxamide

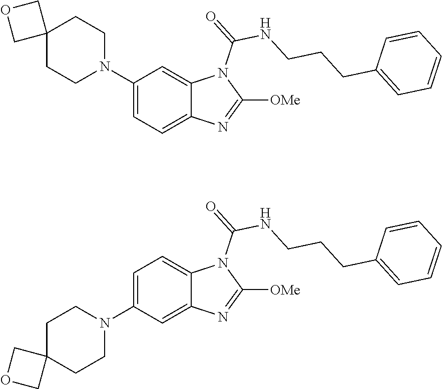 [SMILES: COc1nc2ccc(N3CCC4(CC3)COC4)cc2n1C(=O)NCCCc1ccccc1 ; COc1nc2cc(N3CCC4(CC3)COC4)ccc2n1C(=O)NCCCc1ccccc1]

Following general procedure E, 3-fluoro-2-nitroaniline (1.0 g, 6.4 mmol) and 2-oxa-7-azaspiro[3.5]nonane (980 mg, 7.69 mmol) afforded 2-nitro-5-(2-oxa-7-azaspiro[3.5]nonan-7-yl)aniline (930 mg, 55.1%) as an orange solid. LC-MS m/z: 263.3[M+H]⁺. Purity (214 nm): 85.31%; t_{R}=1.13 min.

Following general procedure F, 2-nitro-5-(2-oxa-7-azaspiro[3.5]nonan-7-yl)aniline (930 mg, 3.42 mmol) afforded 4-(2-oxa-7-azaspiro[3.5]nonan-7-yl)benzene-1,2-diamine (800 mg, 97%) as a black solid. LC-MS m/z: 233.31 [M+H]⁺. Purity (214 nm): 85%; t_{R}=1.37 min.

Following general procedure G (method A), 4-(2-oxa-7-azaspiro[3.5]nonan-7-yl)benzene-1,2-diamine (400 mg, 1.71 mmol) afforded 7-(2-methoxy-1H-benzo[d]imidazol-5-yl)-2-oxa-7-azaspiro[3.5]nonane (250 mg, 53.2%) as a black solid. LC-MS m/z: 273.33 [M+H]⁺. Purity (214 nm): 88.5%; t_{R}=1.48 min.

Following general procedure C, 7-(2-methoxy-1H-benzo[d]imidazol-5-yl)-2-oxa-7-azaspiro[3.5]nonane (200 mg, 0.73 mmol) and 3-phenylpropan-1-amine (150 mg, 1.1 mmol) afforded 2-methoxy-N-(3-phenylpropyl)-6-(2-oxa-7-azaspiro[3.5]nonan-7-yl)-1H-benzo[d]imidazole-1-carboxamide (19.1 mg, 6.0%) as a white solid and 2-methoxy-N-(3-phenylpropyl)-5-(2-oxa-7-azaspiro[3.5]nonan-7-yl)-1H-benzo[d]-imidazole-1-carboxamide (40.3 mg, 12.7%) as a colorless oil.

2-methoxy-N-(3-phenylpropyl)-6-(2-oxa-7-azaspiro[3.5]nonan-7-yl)-1H-benzo[d]imidazole-1-carboxamide: ¹H NMR (400 MHz, CDCl₃) δ 7.83 (d, J=2.3 Hz, 1H), 7.36 (d, J=8.7 Hz, 1H), 7.32-7.27 (m, 2H), 7.21 (d, J=6.0 Hz, 3H), 6.98-6.90 (m, 2H), 4.48 (s, 4H), 4.27 (s, 3H), 3.46 (dd, J=13.2, 6.9 Hz, 2H), 3.10 (t, J=8.0 Hz, 4H), 2.73 (t, J=7.6 Hz, 2H), 2.09-1.99 (m, 6H). LC-MS m/z: 434.53 [M+H]⁺. HPLC Purity (214 nm): 100%; t_{R}=6.45 min.

2-methoxy-N-(3-phenylpropyl)-5-(2-oxa-7-azaspiro[3.5]nonan-7-yl)-1H-benzo[d]imidazole-1-carboxamide: ¹H NMR (400 MHz, CDCl₃) δ 8.00 (d, J=8.9 Hz, 1H), 7.30 (d, J=7.8 Hz, 2H), 7.21 (d, J=6.9 Hz, 3H), 7.07 (d, J=2.3 Hz, 1H), 6.90 (dd, J=8.9, 2.4 Hz, 2H), 4.48 (s, 4H), 4.28 (s, 3H), 3.46 (dd, J=13.1, 6.1 Hz, 2H), 3.09 (t, J=5.6 Hz, 4H), 2.73 (t, J=7.6 Hz, 2H), 2.05 (t, J=5.6 Hz, 4H), 2.01 (p, J=9.2 Hz, 2H). LC-MS m/z: 434.53 [M+H]⁺. HPLC Purity (214 nm): 100%; t_{R}=6.51 min.

### Example 239—4-(Hexahydropyrrolo[1,2-a]pyrazin-2(1H)-yl)-N-(3-phenylpropyl)-1H-benzo[d]imidazole-1-carboxamide

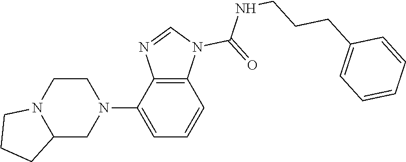 [SMILES: O=C(NCCCc1ccccc1)n1cnc2c(N3CCN4CCCC4C3)cccc21]

Following general procedure E, 3-fluoro-2-nitrobenzenamine (1.00 g, 6.41 mmol) and octahydropyrrolo[1,2-a]pyrazine (0.81 g, 6.41 mmol) afforded 3-(hexahydropyrrolo[1,2-a]pyrazin-2(1H)-yl)-2-nitrobenzenamine (1.0 g, 59.8%) as a yellow solid. LC-MS m/z: 263.2 [M+H]⁺. Purity (214 nm): >99%; t_{R}=1.73 min.

Following general procedure F, 3-(hexahydropyrrolo[1,2-a]pyrazin-2(1H)-yl)-2-nitrobenzenamine (1.00 g, 3.81 mmol) afforded 3-(hexahydropyrrolo[1,2-a]pyrazin-2(1H)-yl)benzene-1,2-diamine (0.6 g, 68.2%) as a yellow solid. LC-MS m/z: 233.2 [M+H]⁺. Purity (214 nm): 78%; t_{R}=1.42 min.

Following general procedure G (method B), 3-(hexahydropyrrolo[1,2-a]pyrazin-2(1H)-yl)benzene-1,2-diamine (0.60 g, 2.58 mmol) afforded 4-(hexahydropyrrolo[1,2-a]pyrazin-2(1H)-yl)-1H-benzo[d]imidazole (0.4 g, 64.5%) as a yellow solid. LC-MS m/z: 243.2 [M+H]⁺. Purity (214 nm): >99%; t_{R}=1.43 min.

Following general procedure C, 4-(hexahydropyrrolo[1,2-a]pyrazin-2(1H)-yl)-1H-benzo[d]imidazole (0.15 g, 0.62 mmol) and 3-phenylpropan-1-amine (0.09 g, 0.68 mmol) afforded the title compound (111 mg, 39.9%) as a white solid. ¹H NMR (400 MHz, CDCl₃) δ 8.55 (s, 1H), 7.44 (d, J=8.4 Hz, 1H), 7.31-7.27 (m, 2H), 7.26-7.18 (m, 4H), 6.78 (bs, 1H), 6.68 (d, J=8.0 Hz, 1H), 4.14 (dd, J=21.2, 10.4 Hz, 2H), 3.56 (q, J=8.2 Hz, 2H), 3.35-3.24 (m, 3H), 3.02 (t, J=7.8 Hz, 1H), 2.28 (bs, 2H), 2.76 (t, J=7.6 Hz, 2H), 2.63 (bs, 1H), 2.10-2.00 (m, 4H), 2.01-1.89 (m, 1H), 1.82-1.73 (m, 1H). LC-MS m/z: 404.2 [M+H]⁺. HPLC Purity (214 nm): 100%; t_{R}=6.39 min.

### Example 240—4-((1R,5S)-8-Methyl-3,8-diazabicyclo[3.2.1]octan-3-yl)-N-(3-phenyl propyl)-1H-benzo[d]imidazole-1-carboxamide

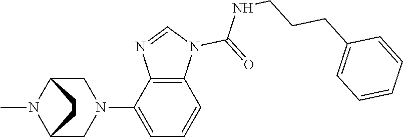

Following general procedure E, (1R,5S)-8-methyl-3,8-diazabicyclo[3.2.1]octane (300 mg, 1.3 mmol) and 3-fluoro-2-nitroaniline (561 mg, 3.6 mmol) afforded 3-((1R,5S)-8-methyl-3,8-diazabicyclo[3.2.1]octan-3-yl)-2-nitroaniline (226 mg, 66.2%). LC-MS m/z: 263.7 [M+H]⁺.

Following general procedure F, 3-((1R,5S)-8-methyl-3,8-diazabicyclo[3.2.1]octan-3-yl)-2-nitroaniline (226 g, 0.86 mmol) afforded 3-((1R,5S)-8-methyl-3,8-diazabicyclo[3.2.1]octan-3-yl)benzene-1,2-diamine (150 mg, 69.8%) which was used directly in the next step. LC-MS m/z: 233.7 [M+H]⁺.

Following general procedure G (method B), 3-((1R,5S)-8-methyl-3,8-diazabicyclo[3.2.1]octan-3-yl)benzene-1,2-diamine (150 mg, 0.6 mmol) afforded 4-((1R,5S)-8-methyl-3,8-diazabicyclo-[3.2.1]octan-3-yl)-1H-benzo[d]imidazole (80 mg, 66.7%) which was used directly in the next step. LC-MS m/z: 243.1 [M+H]⁺.

Following general procedure C, 4-((1R,5S)-8-methyl-3,8-diazabicyclo[3.2.1]octan-3-yl)-1H-benzo[d]imidazole (80 mg, 0.3 mmol) and 3-phenylpropan-1-amine (40 mg, 0.3 mmol) afforded the title compound (17.0 mg, 12.8%) as a white solid. ¹H NMR (400 MHz, CDCl₃) δ 8.66 (s, 1H), 7.48 (bs 1H), 7.38-7.08 (m, 7H), 6.51 (d, J=7.7 Hz, 1H), 4.15 (d, J=11.6 Hz, 2H), 3.90 (bs, 2H), 3.81 (bs, 2H), 3.59-3.52 (m, 2H), 2.83 (s, 3H), 2.77 (t, J=7.6 Hz, 2H), 2.44 (bs, 2H), 2.25 (bs, 2H), 2.10 (p, J=6.8 Hz, 2H). LC-MS m/z: 403.7 [M+H]⁺. Purity (214 nm): 100%; t_{R}=7.10 min.

### Example 241—4-(3-Methyl-3,8-diazabicyclo[3.2.1]octan-8-yl)-N-(3-phenylpropyl)-1H-benzo[d]imidazole-1-carboxamide

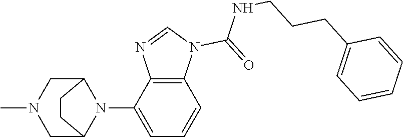 [SMILES: CN1CC2CCC(C1)N2c1cccc2c1ncn2C(=O)NCCCc1ccccc1]

Following general procedure E, 3-fluoro-2-nitroaniline (500.0 mg, 3.2 mmol) and tert-butyl 3,8-diazabicyclo[3.2.1]octane-3-carboxylate (800.0 mg, 3.8 mmol) afforded tert-butyl 8-(3-amino-2-nitrophenyl)-3,8-diazabicyclo[3.2.1]octane-3-carboxylate (420.0 mg, 37.7%) as a yellow oil. LC-MS m/z: 349.2 [M+H]⁺. Purity (254 nm): 98.0%; t_{R}=2.04 min.

Following general procedure F, tert-butyl 8-(3-amino-2-nitrophenyl)-3,8-diazabicyclo[3.2.1]octane-3-carboxylate (420.0 g, 1.1 mmol) afforded tert-butyl 8-(2,3-diaminophenyl)-3,8-diazabicyclo[3.2.1]octane-3-carboxylate (330.0 mg, 94.3%) as a brown oil. LC-MS m/z: 319.2 [M+H]⁺. Purity (254 nm): 94.0%; t_{R}=1.94 min.

A mixture of tert-butyl 8-(2,3-diaminophenyl)-3,8-diazabicyclo[3.2.1]octane-3-carboxylate (280.0 mg, 0.9 mmol) and AcOH (5.0 mL) was stirred at 100° C. for 2 h and then concentrated in vacuo to give a residue which was partitioned between NaHCO₃ aqueous solution (30 mL) and EA (50 mL). The organic layer was dried over Na₂SO₄, filtered, concentrated and purified by silica gel column chromatography (EA:PE=2:3) to give 4-(3,8-diazabicyclo[3.2.1]octan-8-yl)-1H-benzo[d]imidazole (190.0 mg, 92.6%) as a yellow oil. LC-MS m/z: 229.2 [M+H]⁺. Purity (214 nm): 99.1%; t_{R}=1.29 min.

Following general procedure E, 4-(3,8-diazabicyclo[3.2.1]octan-8-yl)-1H-benzo[d]imidazole (200.0 mg, 0.9 mmol) and MeI (100.0 mg, 0.7 mmol) afforded 4-(3-methyl-3,8-diazabicyclo[3.2.1]octan-8-yl)-1H-benzo[d]imidazole (140.0 mg, 64.3%) as a yellow oil. LC-MS m/z: 243.2 [M+H]⁺. Purity (214 nm): 82.8%, t_{R}=1.48 min.

Following general procedure C, 4-(3-methyl-3,8-diazabicyclo[3.2.1]octan-8-yl)-1H-benzo[d]imidazole (140.0 mg, 0.4 mmol) and 3-phenylpropan-1-amine (70.0 mg, 0.5 mmol) afforded the title compound (11.9 mg, 1.59%) as a white solid. ¹H NMR (400 MHz, CDCl₃) δ 8.25 (s, 1H), 7.34-7.28 (m, 2H), 7.26-7.18 (m, 5H), 6.63 (d, J=7.3 Hz, 1H), 6.16 (s, 1H), 5.03 (s, 2H), 3.56 (dd, J=12.8, 6.9 Hz, 2H), 3.12 (d, J=10.9 Hz, 2H), 2.80-2.74 (m, 4H), 2.39 (s, 3H), 2.25-2.19 (m, 4H), 2.05 (p, J=7.5 Hz, 2H). LC-MS m/z: 404.2 [M+H]⁺. HPLC Purity (214 nm): 96.78%; t_{R}=8.67 min.

### Example 242—N-(3-phenylpropyl)-4-(2-(trifluoromethyl)aziridin-1-yl)-1H-benzo[d]imidazole-1-carboxamide

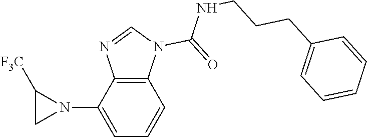 [SMILES: O=C(NCCCc1ccccc1)n1cnc2c(N3CC3C(F)(F)F)cccc21]

Following general procedure C, 4-(2-(trifluoromethyl)aziridin-1-yl)-1H-benzo[d]imidazole (47 mg, 0.21 mmol) and 3-phenylpropan-1-amine (42 mg, 0.31 mmol) afforded the title compound (32.9 mg, 39.5%) as a white solid. ¹H NMR (400 MHz, CDCl₃) δ 8.16 (s, 1H), 7.40 (dd, J=8.0 Hz, 0.8 Hz, 1H), 7.36-7.29 (m, 3H), 7.24-7.20 (m, 3H), 6.96 (dd, J=8.0 Hz, 0.4 Hz, 1H), 5.55 (t, J=4.0 Hz, 1H), 3.56 (q, J=6.8 Hz, 2H), 3.01-2.97 (m, 1H), 2.87 (d, J=3.2 Hz, 1H), 2.79 (t, J=7.2 Hz, 2H), 2.52 (d, J=6.0 Hz, 1H), 2.06 (p, J=7.5 Hz, 2H). LC-MS m/z: 389.0 [M+H]⁺. HPLC Purity (214 nm): 99%; t_{R}=9.32 min.

### Example 243—4-(6-Methyl-3,6-diazabicyclo[3.1.1]heptan-3-yl)-N-(3-phenylpropyl)-1H-benzo[d]imidazole-1-carboxamide

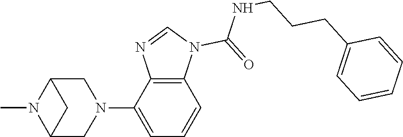 [SMILES: CN1C2CC1CN(c1cccc3c1ncn3C(=O)NCCCc1ccccc1)C2]

Following general procedure C, 3-(1H-benzo[d]imidazol-4-yl)-6-methyl-3,6-diazabicyclo[3.1.1]heptane (105 mg, 0.46 mmol) and 3-phenylpropan-1-amine (62 mg, 0.46 mmol) afforded the title compound (15.0 mg, 8.0%) as a yellow solid. 1H NMR (400 MHz, CDCl₃) δ 8.69 (s, 1H), 8.55 (s, 1H), 8.34 (bs, 1H), 7.64 (d, J=8.1 Hz, 1H), 7.33-7.17 (m, 5H), 6.31 (d, J=7.7 Hz, 1H), 4.77 (bs, 2H), 3.74 (d, J=11.9 Hz, 2H), 3.55-3.42 (m, 4H), 2.97-2.85 (m, 1H), 2.76 (t, J=7.5 Hz, 2H), 2.41 (bs, 4H), 2.06 (p, J=7.2 Hz, 2H). LC-MS m/z: 390.0 [M+H]⁺. HPLC Purity (214 nm): 100%; t_{R}=9.15 min.

### Example 244—4-((1S,4S)-5-Methyl-2,5-diazabicyclo[2.2.1]heptan-2-yl)-N-(3-phenyl propyl)-1H-benzo[d]imidazole-1-carboxamide

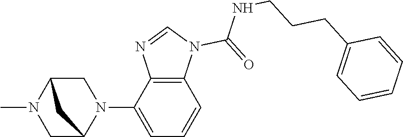 [SMILES: CN1C[C@@H]2C[C@H]1CN2c1cccc2c1ncn2C(=O)NCCCc1ccccc1]

Following general procedure E, 3-fluoro-2-nitroaniline (250 mg, 1.6 mmol) and (1S,4S)-2-methyl-2,5-diazabicyclo[2.2.1]heptane dihydrobromide (436 mg, 1.6 mmol) afforded 3-((1S,4S)-5-methyl-2,5-diazabicyclo[2.2.1]heptan-2-yl)-2-nitroaniline (340 mg, 80%) as a yellow solid. LC-MS m/z: 249.2 [M+H]⁺. Purity (214 nm): 96%; t_{R}=1.43 min.

Following general procedure F, 3-((1S,4S)-5-methyl-2,5-diazabicyclo[2.2.1]heptan-2-yl)-2-nitroaniline (340 mg, 1.37 mmol) afforded 3-((1S,4S)-5-methyl-2,5-diazabicyclo[2.2.1]heptan-2-yl)benzene-1,2-diamine (290 mg, 97%) as a yellow oil. LC-MS m/z: 228.1 [M+H]⁺; t_{R}=0.17 min.

Following general procedure G (method A), 3-((1S,4S)-5-methyl-2,5-diazabicyclo[2.2.1]heptan-2-yl)benzene-1,2-diamine (290 mg, 1.33 mmol) afforded 4-((1S,4S)-5-methyl-2,5-diazabicyclo[2.2.1]heptan-2-yl)-1H-benzo[d]imidazole (30 mg, 10%) as a yellow oil. LC-MS m/z: 229.0 [M+H]⁺. Purity (214 nm): 65%; t_{R}=1.29 min.

Following general procedure C, 4-((1S,4S)-5-methyl-2,5-diazabicyclo[2.2.1]heptan-2-yl)-1H-benzo[d]imidazole (30 mg, 0.13 mmol) and 3-phenylpropan-1-amine (18 mg, 0.13 mmol) afforded the title compound (30.2 mg, 58.9%) as a yellow solid. ¹H NMR (400 MHz, CDCl₃) δ 8.31 (s, 1H), 7.29 (dd, J=13.3, 5.9 Hz, 2H), 7.27-7.22 (m, 5H), 6.75 (bs, 1H), 6.36 (bs, 1H), 5.63 (s, 1H), 4.17 (bs, 1H), 3.84 (dd, J=30.3, 10.4 Hz, 2H), 3.44 (bs 1H), 3.16 (bs, 1H), 2.75 (t, J=7.6 Hz, 2H), 2.72 (s, 3H), 2.39-2.15 (m, 2H), 2.06 (p, J=6.8 Hz, 2H). LC-MS m/z: 390.2 [M+H]⁺. HPLC Purity (214 nm): 100%; t_{R}=8.01 min.

### Example 245—4-(3-Methyl-3,6-diazabicyclo[3.1.1]heptan-6-yl)-N-(3-phenylpropyl)-1H-benzo[d]imidazole-1-carboxamide

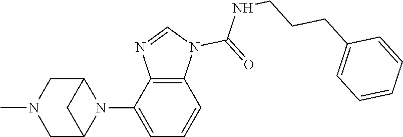 [SMILES: CN1CC2CC(C1)N2c1cccc2c1ncn2C(=O)NCCCc1ccccc1]

Following general procedure E, 3-fluoro-2-nitroaniline (150 mg, 1.34 mmol) and 3-methyl-3,6-diazabicyclo[3.1.1]heptane (209 mg, 1.34 mmol) afforded 3-(3-methyl-3,6-diazabicyclo[3.1.1]-heptan-6-yl)-2-nitroaniline (205 mg, 62%) as a yellow solid. LC-MS m/z: 249.2 [M+H]⁺; t_{R}=1.62 min.

Following general procedure F, 3-(3-methyl-3,6-diazabicyclo[3.1.1]heptan-6-yl)-2-nitroaniline (205 mg, 0.83 mmol) afforded 3-(3-methyl-3,6-diazabicyclo[3.1.1]heptan-6-yl) benzene-1,2-diamine (180 mg, 99%) as a yellow oil. LC-MS m/z: 219.2 [M+H]⁺; t_{R}=1.21 min.

Following general procedure G (method B), 3-(3-methyl-3,6-diazabicyclo[3.1.1]heptan-6-yl)benzene-1,2-diamine (180 mg, 0.73 mmol) afforded 6-(1H-benzo[d]imidazol-4-yl)-3-methyl-3,6-diazabicyclo[3.1.1]heptane (80 mg, 43%) as a yellow solid. LC-MS m/z: 229.2 [M+H]⁺; t_{R}=1.32 min.

Following general procedure C, 6-(1H-benzo[d]imidazol-4-yl)-3-methyl-3,6-diazabicyclo[3.1.1]heptane (80 mg, 0.35 mmol) and 3-phenylpropan-1-amine (47 mg, 0.35 mmol) afforded the title compound (20.4 mg, 14.9%) as a white solid. ¹H NMR (400 MHz, CDCl₃) δ 8.53 (bs, 1H), 8.48 (s, 1H), 7.43 (d, J=8.4 Hz, 1H), 7.37-7.10 (m, 6H), 6.32 (d, J=7.7 Hz, 1H), 4.75 (bs, 2H), 3.72 (d, J=11.6 Hz, 2H), 3.54 (d, J=5.2 Hz, 2H), 3.40 (d, J=12.0 Hz, 2H), 2.90 (bs, 1H), 2.77 (t, J=7.3 Hz, 2H), 2.40 (bs, 4H), 2.06 (p, J=7.3 Hz, 2H). LC-MS m/z: 390.1 [M+H]⁺. HPLC Purity (214 nm): 100%; t_{R}=6.28 min.

### Example 246—4-((1R,4R)-5-Methyl-2,5-diazabicyclo[2.2.1]heptan-2-yl)-N-(3-phenyl propyl)-1H-benzo[d]imidazole-1-carboxamide

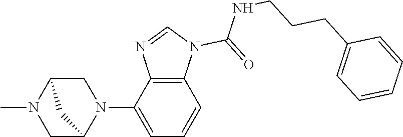 [SMILES: CN1C[C@H]2C[C@@H]1CN2c1cccc2c1ncn2C(=O)NCCCc1ccccc1]

Following general procedure E, 3-fluoro-2-nitroaniline (250 mg, 1.6 mmol) and (1R,4R)-2-methyl-2,5-diazabicyclo[2.2.1]heptane dihydrochloride (297 mg, 1.6 mmol) afforded 3-((1R,4R)-5-methyl-2,5-diazabicyclo[2.2.1]heptan-2-yl)-2-nitroaniline (330 mg, 83%) as an orange solid. LC-MS m/z: 249.2 [M+H]⁺. Purity (214 nm): 95%; t_{R}=1.32 min.

Following general procedure F, 3-((1R,4R)-5-methyl-2,5-diazabicyclo[2.2.1]heptan-2-yl)-2-nitroaniline (330 mg, 1.33 mmol) afforded 3-((1R,4R)-5-methyl-2,5-diazabicyclo[2.2.1]heptan-2-yl)benzene-1,2-diamine (280 mg, 97%) as a gray oil. LC-MS m/z: 219.2 [M+H]⁺; t_{R}=1.07 min.

Following general procedure G (method B), 3-((1R,4R)-5-methyl-2,5-diazabicyclo[2.2.1]heptan-2-yl)benzene-1,2-diamine (260 mg, 1.09 mmol) afforded 4-((1R,4R)-5-methyl-2,5-diazabicyclo[2.2.1]heptan-2-yl)-1H-benzo[d]imidazole (290 mg, 98%) as a brown oil. LC-MS m/z: 229.2 [M+H]⁺. Purity (214 nm): 90%; t_{R}=1.07 min.

Following general procedure C, 4-((1R,4R)-5-methyl-2,5-diazabicyclo[2.2.1]heptan-2-yl)-1H-benzo[d]imidazole (140 mg, 0.61 mmol) and 3-phenylpropan-1-amine (83 mg, 0.61 mmol) afforded the title compound (15.0 mg, 6.2%) as a yellow solid. ¹H NMR (400 MHz, CDCl₃) δ 8.38 (s, 1H), 7.36-7.27 (m, 3H), 7.24-7.15 (m, 4H), 6.34 (d, J=7.8 Hz, 1H), 5.63 (bs, 1H), 4.18 (s, 1H), 3.83 (dd, J=17.8, 5.6 Hz, 2H), 3.55-3.42 (m, 2H), 3.36 (bs, 1H), 3.17 (bs, 1H), 2.75 (t, J=7.2 Hz, 2H), 2.70 (s, 3H), 2.25 (dd, J=21.9, 9.6 Hz, 2H), 2.05 (p, J=6.8 Hz, 2H). LC-MS m/z: 390.0 [M+H]⁺. HPLC Purity (214 nm): 100%; t_{R}=8.63 min.

### Example 247—4-(5-Methyl-2,5-diazabicyclo[2.2.2]octan-2-yl)-N-(3-phenylpropyl)-1H-benzo[d]imidazole-1-carboxamide

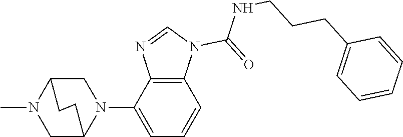 [SMILES: CN1CC2CCC1CN2c1cccc2c1ncn2C(=O)NCCCc1ccccc1]

Following general procedure E, 3-fluoro-2-nitroaniline (390 mg, 2.49 mmol) and 2-methyl-2,5-diazabicyclo[2.2.2]octane (400 mg, 1.94 mmol) afforded 3-(5-methyl-2,5-diazabicyclo[2.2.2]-octan-2-yl)-2-nitroaniline (450 mg, 88%) as a red solid. LC-MS m/z: 263.2 [M+H]⁺. Purity (214 nm): 96%; t_{R}=1.42 min.

Following general procedure F, 3-(5-methyl-2,5-diazabicyclo[2.2.2]octan-2-yl)-2-nitroaniline (450 mg, 1.72 mmol) afforded 3-(5-methyl-2,5-diazabicyclo[2.2.2]octan-2-yl) benzene-1,2-diamine (300 mg, 75%) as a colorless oil. LC-MS m/z: 233.2 [M+H]⁺; t_{R}=1.12 min.

Following general procedure G (method B), 3-(5-methyl-2,5-diazabicyclo[2.2.2]octan-2-yl)benzene-1,2-diamine (300 mg, 1.29 mmol) afforded 2-(1H-benzo[d]imidazol-4-yl)-5-methyl-2,5-diazabicyclo[2.2.2]octane (258 mg, 83%) as a yellow oil. LC-MS m/z: 243.2 [M+H]⁺. Purity (214 nm): 95%; t_{R}=1.24 min.

Following general procedure C, 2-(1H-benzo[d]imidazol-4-yl)-5-methyl-2,5-diazabicyclo[2.2.2]octane (258 mg, 1.06 mmol) and 3-phenylpropan-1-amine (144 mg, 1.06 mmol) afforded the title compound (39.9 mg, 9.4%) as a yellow solid. ¹H NMR (400 MHz, CDCl₃) δ 8.52 (s, 1H), 7.62 (bs, 1H), 7.41 (d, J=8.1 Hz, 1H), 7.33-7.27 (m, 2H), 7.25-7.18 (m, 4H), 6.43 (d, J=7.9 Hz, 1H), 5.49 (s, 1H), 4.13 (d, J=11.4 Hz, 1H), 3.71 (d, J=8.1 Hz, 1H), 3.61-3.50 (m, 4H), 3.28 (d, J=10.0 Hz, 1H), 2.81 (s, 3H), 2.75 (t, J=7.6 Hz, 2H), 2.46 (bs, 1H), 2.19 (bs, 1H), 2.08 (p, J=7.4 Hz, 2H), 2.07-1.93 (m, 2H). LC-MS m/z: 404.3 [M+H]⁺. HPLC Purity (214 nm): 100%; t_{R}=9.49 min.

### Example 248—4-(4,4-Difluoropiperidin-1-yl)-N-(3-phenylpropyl)-1H-benzo[d]imidazole-1-carboxamide

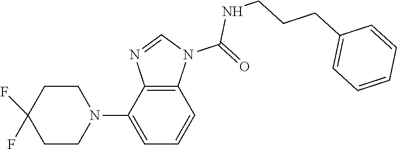 [SMILES: O=C(NCCCc1ccccc1)n1cnc2c(N3CCC(F)(F)CC3)cccc21]

Following general procedure C, 4-(4,4-difluoropiperidin-1-yl)-1H-benzo[d]imidazole (100 mg, 0.42 mmol) and 3-phenylpropan-1-amine (85 mg, 0.63 mmol) afforded the title compound (50.5 mg, 30%) as a white solid. ¹H NMR (400 MHz, CDCl₃) δ 8.18 (s, 1H), 7.30 (dd, J=9.9, 4.9 Hz, 2H), 7.26-7.17 (m, 5H), 6.79-6.70 (m, 1H), 5.63 (t, J=5.1 Hz, 1H), 3.71-3.61 (m, 4H), 3.56 (dd, J=12.9, 6.9 Hz, 2H), 2.78 (t, J=7.4 Hz, 2H), 2.22 (ddd, J=19.6, 13.7, 5.7 Hz, 4H), 2.07 (p, J=7.2 Hz, 2H). LC-MS m/z: 399.1 [M+H]⁺. HPLC Purity (214 nm): 99%; t_{R}=9.47 min.

### Example 249—4-(4-Ethylpiperazin-1-yl)-N-(3-phenylpropyl)-1H-benzo[d]imidazole-1-carboxamide

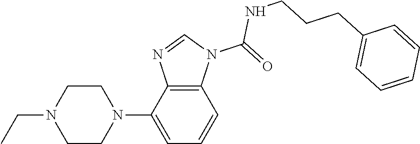 [SMILES: CCN1CCN(c2cccc3c2ncn3C(=O)NCCCc2ccccc2)CC1]

Following general procedure C, 4-(4-ethylpiperazin-1-yl)-1H-benzo[d]imidazole (240 mg, 1.0 mmol) and 3-phenylpropan-1-amine (135 mg, 1.0 mmol) afforded the title compound (22.5 mg, 5.8%) as a white solid. ¹H NMR (400 MHz, CDCl₃) δ 8.36 (s, 1H), 7.41 (d, J=8.0 Hz, 1H), 7.33-7.08 (m, 6H), 6.72 (d, 0.1_7.9 Hz, 1H), 6.52 (bs, 1H), 3.70 (bs, 4H), 3.54 (dd, J=12.9, 6.9 Hz, 2H), 3.14 (bs, 4H), 2.88 (q, J=7.3 Hz, 2H), 2.77 (t, J=7.5 Hz, 2H), 2.07 (p, J=7.2 Hz, 2H), 1.31 (t, J=7.3 Hz, 3H). LC-MS m/z: 391.7 [M+H]⁺. HPLC Purity (214 nm): 100%; t_{R}=6.26 min.

### Example 250—4-(4-Methyl-4,7-diazaspiro[2.5]octan-7-yl)-N-(3-phenylpropyl)-1H-benzo[d]imidazole-1-carboxamide

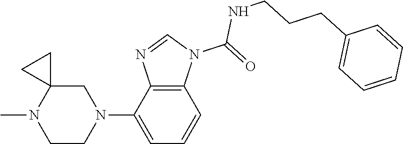 [SMILES: CN1CCN(c2cccc3c2ncn3C(=O)NCCCc2ccccc2)CC12CC2]

Following general procedure E, 3-fluoro-2-nitroaniline (312 mg, 2.0 mmol) and tert-butyl 4,7-diazaspiro[2.5]octane-4-carboxylate (424 mg, 2 mmol) afforded tert-butyl 7-(3-amino-2-nitrophenyl)-4,7-diazaspiro[2.5]octane-4-carboxylate (470 mg, crude) as a yellow solid. LC-MS m/z: 349.1 [M+H]⁺. Purity (214 nm): 82.9%; t_{R}=1.53 min.

Following general procedure F, tert-butyl 7-(3-amino-2-nitrophenyl)-4,7-diazaspiro[2.5]octane-4-carboxylate (170 mg, 0.5 mmol) afforded crude tert-butyl 7-(2,3-diaminophenyl)-4,7-diazaspiro[2.5]octane-4-carboxylate (160 mg) as a yellow oil which was used in the next step. LC-MS m/z: 319.3 [M+H]⁺. Purity (214 nm): 92.9%; t_{R}=1.24 min.

Following general procedure G (method A), tert-butyl 7-(2,3-diaminophenyl)-4,7-diazaspiro[2.5]octane-4-carboxylate (160 mg, 0.5 mmol) afforded crude 4-(4,7-diazaspiro[2.5]octan-7-yl)-1H-benzo[d]imidazole (160 mg) as a yellow oil which was used directly in the next step. LC-MS m/z: 229.2 [M+H]⁺. Purity (214 nm): 89.4%; t_{R}=1.44 min.

A mixture of 4-(4,7-diazaspiro[2.5]octan-7-yl)-1H-benzo[d]imidazole (114 mg, 0.5 mmol), HCHO (71 mg, 0.7 mmol) and NaBH₃CN (94 mg, 1.5 mmol) in MeOH (10 mL) was stirred at RT for 3 h. Then cooled and concentrated in vacuo to give a residue which was purified by silica gel column chromatography (DCM:MeOH=20:1) to give 4-(4-methyl-4,7-diazaspiro[2.5]octan-7-yl)-1H-benzo[d]imidazole (160 mg, crude) as a yellow oil. LC-MS m/z: 243.2 [M+H]⁺. HPLC Purity (214 nm): 88.6%; t_{R}=1.36 min.

Following general procedure C, 4-(4-methyl-4,7-diazaspiro[2.5]octan-7-yl)-1H-benzo[d]imidazole (121 mg, 0.5 mmol) and 3-phenylpropan-1-amine (51.0 mg, 0.5 mmol) afforded the title compound (53.4 mg, 35.6%) as a yellow solid. ¹H NMR (400 MHz, CDCl₃) δ 8.19 (s, 1H), 7.44-7.26 (m, 4H), 7.25-7.14 (m, 3H), 6.69 (d, J=7.5 Hz, 11H), 5.94 (bs, 1H), 3.76-3.63 (m, 2H), 3.60-3.53 (m, 4H), 3.44-3.33 (m, 2H), 2.79 (t, J=6.8 Hz, 2H), 2.70 (s, 3H), 2.08 (p, J=7.2 Hz, 2H), 1.30-1.08 (m, 2H), 0.90 (t, J=6.3 Hz, 2H). LC-MS m/z: 404.2 [M+H]⁺. HPLC Purity (214 nm): 100%; t_{R}=6.42 min.

### Example 251—4-(4-(1-Methylazetidin-3-yl)piperazin-1-yl)-N-(3-phenylpropyl)-1H-benzo[d]imidazole-1-carboxamide

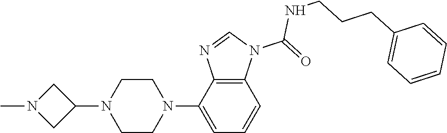 [SMILES: CN1CC(N2CCN(c3cccc4c3ncn4C(=O)NCCCc3ccccc3)CC2)C1]

To a solution of 4-(piperazin-1-yl)-1H-benzo[d]imidazole (202 mg, 1 mmol) in MeOH (10 mL) was added 1-methylazetidin-3-one (140 mg, 1.2 mmol) and Et₃N (120 mg, 1.2 mmol). The mixture was stirred at RT for 1 h and then NaBH₃CN (190 mg, 3 mmol) was added. The resulting mixture was stirred at 50° C. for 16 h. The mixture was concentrated to afford a residue which was purified by silica gel column chromatography (DCM:MeOH=1:10) to afford 4-(4-(1-methylazetidin-3-yl)piperazin-1-yl)-1H-benzo[d]imidazole (170 mg, 62.9/o) as a yellow oil. LC-MS m/z: 272.2 [M+H]⁺. Purity (214 nm): 82.25%; t_{R}=1.34 min.

Following general procedure C, 4-(4-(1-methylazetidin-3-yl)piperazin-1-yl)-1H-benzo[d]imidazole (170 mg, 0.63 mmol) and 3-phenylpropan-1-amine (85 mg, 0.63 mmol) afforded the title compound (7.1 mg, 2.6%) as a white solid. ¹H NMR (400 MHz, CDCl₃) δ 8.56 (s, 1H), 7.45 (d, J=8.4 Hz, 1H), 7.33-7.27 (m, 2H), 7.26-7.21 (m, 4H), 6.94 (t, J=4.8 Hz, 1H), 6.69 (d, J=8.0 Hz, 1H), 4.15 (t, J=7.9 Hz, 2H), 3.60-3.39 (m, 8H), 3.37-3.26 (m, 1H), 2.76 (t, J=7.5 Hz, 2H), 2.71 (s, 3H), 2.60-2.49 (m, 4H), 2.05 (p, J=7.6 Hz, 2H). HPLC Purity (214 nm): =100%; t_{R}=6.10 min.

### Example 252—4-(4-Cyclopropyl-3-oxopiperazin-1-yl)-N-(3-phenylpropyl)-1H-benzo[d]imidazole-1-carboxamide

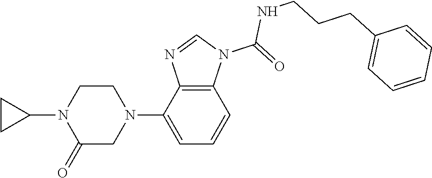 [SMILES: O=C1CN(c2cccc3c2ncn3C(=O)NCCCc2ccccc2)CCN1C1CC1]

Following general procedure E, 1-cyclopropylpiperazin-2-one (270 mg, 1.9 mmol) and 3-fluoro-2-nitroaniline (237 mg, 1.5 mmol) afforded 4-(3-amino-2-nitrophenyl)-1-cyclopropyl piperazin-2-one (40 mg, 9.3%) as a red solid. LC-MS m/z: 277.1 [M+H]⁺. Purity (214 nm): 61.98%; t_{R}=1.68 min.

Following general procedure F, 4-(3-amino-2-nitrophenyl)-1-cyclopropylpiperazin-2-one (40 mg, 0.15 mmol) afforded 1-cyclopropyl-4-(2,3-diaminophenyl)piperazin-2-one (30 mg, 84.3%) as a red oil. LC-MS m/z: 247.2 [M+H]⁺. Purity (214 nm): 76.19%; t_{R}=0.92 min.

Following general procedure G (method B), 1-cyclopropyl-4-(2,3-diaminophenyl) piperazin-2-one (30 mg, 0.12 mmol) afforded 4-(1H-benzo[d]imidazol-4-yl)-1-cyclopropyl piperazin-2-one (30 mg, 96.2%) as a red oil. LC-MS m/z: 257.2 [M+H]⁺. Purity (214 nm): 83.87%; t_{R}=0.75 min.

Following general procedure C, 4-(1H-benzo[d]imidazol-4-yl)-1-cyclopropylpiperazin-2-one (30 mg, 0.12 mmol) and 3-phenylpropan-1-amine (16 mg, 0.12 mmol) afforded the title compound (15.5 mg, 31.0%) as a white solid. ¹H NMR (400 MHz, CDCl₃) δ 8.21 (s, 1H), 7.44-7.29 (m, 4H), 7.27-7.08 (m, 3H), 6.67 (dd, J=7.2, 1.5 Hz, 1H), 5.87 (bs, 1H), 4.08 (s, 2H), 3.98 (t, J=5.6 Hz, 2H), 3.56 (q, J=7.4 Hz, 2H), 3.51 (t, J=5.6 Hz, 2H), 2.80 (t, J=5.3 Hz, 3H), 2.05 (p, J=7.6 Hz, 2H), 1.91-1.85 (m, 2H), 0.97-0.64 (m, 2H). LC-MS m/z: 418.0 [M+H]⁺. HPLC Purity (254 nm): 100%; t_{R}=8.48 min.

### Examples 253a and 253b—(S)-4-(4-Methylpiperazin-1-yl)-N-(3-phenylbutyl)-1H-benzo[d]imidazole-1-carboxamide and (R)-4-(4-methylpiperazin-1-yl)-N-(3-phenylbutyl)-1H-benzo[d]imidazole-1-carboxamide

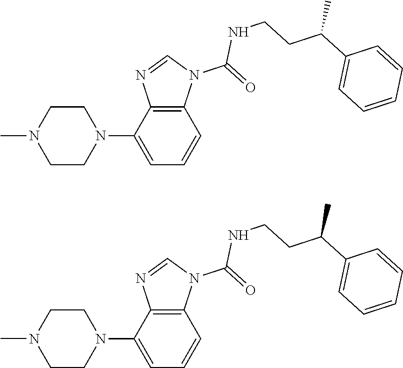 [SMILES: C[C@@H](CCNC(=O)n1cnc2c(N3CCN(C)CC3)cccc21)c1ccccc1 ; C[C@H](CCNC(=O)n1cnc2c(N3CCN(C)CC3)cccc21)c1ccccc1]

Following general procedure C, 4-(4-methylpiperazin-1-yl)-1H-benzo[d]imidazole (250 mg, 1.16 mmol) and 3-phenyl-1-butylamine (5 mg, 0.09 mmol) afforded after supercritical fluid chromatography (S)-4-(4-methylpiperazin-1-yl)-N-(3-phenylbutyl)-1H-benzo[d]imidazole-1-carboxamide (29.8 mg, 6.6%) and (R)-4-(4-methylpiperazin-1-yl)-N-(3-phenylbutyl)-1H-benzo[d]imidazole-1-carboxamide (25.8 mg, 5.7%) both as yellow solids.

(S)-4-(4-methylpiperazin-1-yl)-N-(3-phenylbutyl)-1H-benzo[d]imidazole-1-carboxamide: ¹H NMR (400 MHz, CDCl₃) δ 8.20 (s, 1H), 7.35-7.26 (m, 3H), 7.25-7.18 (m, 4H), 6.71 (d, J=8.0 Hz, 1H), 6.14 (bs, 1H), 3.66 (bs, 3H), 3.54-3.48 (m, 2H), 3.39-3.32 (m, 1H), 3.10 (bs, 4H), 2.88-2.82 (m, 1H), 2.61 (s, 3H), 2.07-1.95 (m, 2H), 1.33 (d, J=6.8 Hz, 3H). LC-MS m/z: 392.0 [M+H]⁺. HPLC Purity (214 nm): 100.00%; t_{R}=6.43 min.

(R)-4-(4-methylpiperazin-1-yl)-N-(3-phenylbutyl)-1H-benzo[d]imidazole-1-carboxamide: ¹H NMR (400 MHz, CDCl₃) δ 8.17 (s, 1H), 7.33-7.29 (m, 3H), 7.25-7.18 (m, 4H), 6.72 (d, J=7.6 Hz, 1H), 5.97 (bs, 1H), 3.67 (bs, 3H), 3.55-3.48 (m, 2H), 3.34-3.33 (m, 1H), 3.13 (bs, 4H), 2.88-2.83 (m, 1H), 2.63 (s, 3H), 2.06-1.96 (m, 2H), 1.33 (d, J=6.8 Hz, 3H). LC-MS m/z: 392.0 [M+H]⁺. HPLC Purity (214 nm): 100.00%; t_{R}=6.44 min.

### Example 254—4-(4-Methylpiperazin-1-yl)-N-(3-(1-(trifluoromethyl)cyclopropyl)propyl)-1H-benzo[d]imidazole-1-carboxamide

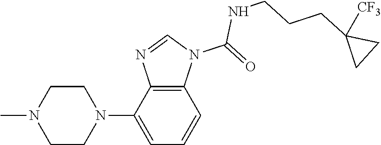 [SMILES: CN1CCN(c2cccc3c2ncn3C(=O)NCCCC2(C(F)(F)F)CC2)CC1]

Following general procedure C, 4-(4-methylpiperazin-1-yl)-1H-benzo[d]imidazole (200 mg, 0.9 mmol) and 2-(3,3-difluorocyclopentyl)ethanamine (190 mg, 0.9 mmol) afforded the title compound (15.0 mg, 4.1%) as a yellow solid. ¹H NMR (400 MHz, DMSO) δ 8.60 (s, 2H), 7.58 (d, J=8.2 Hz, 1H), 7.20 (t, J=8.1 Hz, 1H), 6.68 (d, J=8.0 Hz, 1H), 3.53 (bs, 4H), 3.31-3.21 (m, 2H), 2.73 (bs, 4H), 2.39 (bs, 3H), 1.71-1.62 (m, 4H), 0.90 (q, J=4.8 Hz, 2H), 0.74 (s, 2H). LC-MS m/z: 410.1 [M+H]⁺. HPLC Purity (214 nm): 100%, t_{R}=6.34 min.

### Example 255—4-(4-Methylpiperazin-1-yl)-N-(4,4,4-trifluorobutyl)-1H-benzo[d]imidazole-1-carboxamide

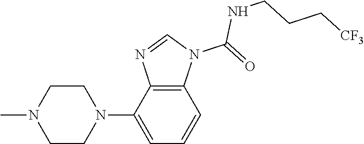 [SMILES: CN1CCN(c2cccc3c2ncn3C(=O)NCCCC(F)(F)F)CC1]

Following general procedure C, 4-(4-methylpiperazin-1-yl)-1H-benzo[d]imidazole (150.0 mg, 0.7 mmol) and 4,4,4-trifluorobutan-1-amine (100.0 mg, 0.8 mmol) afforded the title compound (26.4 mg, 7.0%) as a white solid. ¹H NMR (400 MHz, CDCl₃) δ 8.34 (s, 1H), 7.34-7.27 (m, 2H), 6.76 (dd, J=7.4, 1.4 Hz, 1H), 5.88 (bs, 1H), 3.64-3.57 (m, 4H), 2.78-2.61 (m, 4H), 2.39 (s, 3H), 2.33-2.16 (m, 2H), 2.05-1.97 (m, 4H). LC-MS m/z: 370.0 [M+H]⁺. HPLC Purity (214 nm): 98.73%; t_{R}=6.97 min.

### Example 256—N-((4,4-Difluorocyclohexyl)methyl)-4-(4-methylpiperazin-1-yl)-1H-benzo[d]imidazole-1-carboxamide

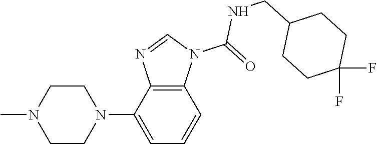 [SMILES: CN1CCN(c2cccc3c2ncn3C(=O)NCC2CCC(F)(F)CC2)CC1]

Following general procedure C, 4-(4-methylpiperazin-1-yl)-1H-benzo[d]imidazole (0.13 g, 0.60 mmol) and (4,4-difluorocyclohexyl)methanamine (0.10 g, 0.66 mmol) afforded the title compound (116.3 mg, 49.5%) as a white solid. ¹H NMR (400 MHz, DMSO-d₆): δ 8.63 (t, J=5.6 Hz, 1H), 8.58 (s, 1H), 7.56 (d, J=7.6 Hz, 1H), 7.19 (t, J=8.0 Hz, 1H), 6.65 (d, J=7.6 Hz, 1H), 3.47 (bs, 4H), 3.21 (t, J=6.0 Hz, 2H), 2.60-2.49 (m, 4H), 2.25 (s, 3H), 2.09-1.98 (m, 2H), 1.89-1.73 (m, 5H), 1.25 (q, J=10.8 Hz, 2H). LC-MS m/z: 392.1 [M+H]⁺. HPLC Purity (214 nm): 100%; t_{R}=5.97 min.

### Example 257—4-(4-Methylpiperazin-1-yl)-N-(2-phenoxyethyl)-1H-benzo[d]imidazole-1-carboxamide

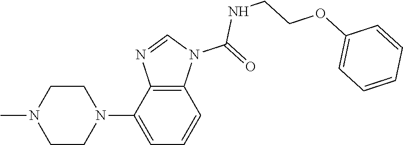 [SMILES: CN1CCN(c2cccc3c2ncn3C(=O)NCCOc2ccccc2)CC1]

Following general procedure C, 4-(4-methylpiperazin-1-yl)-1H-benzo[d]imidazole (200 mg, 0.92 mmol) and 2-phenoxyethanamine (126.2 mg, 0.92 mmol) afforded the title compound (7.5 mg, 3.8%) as a yellow solid. ¹H NMR (400 MHz, CDCl₃) δ 8.36 (s, 1H), 7.34-7.24 (m, 4H), 7.00 (t, J=7.4 Hz, 1H), 6.94 (d, J=7.8 Hz, 2H), 6.75 (dd, J=7.3, 1.5 Hz, 1H), 6.31 (bs, 1H), 4.24 (t, J=5.0 Hz, 2H), 3.94 (dd, J=10.3, 5.4 Hz, 2H), 3.57 (bs, 4H), 2.71 (bs, 4H), 2.40 (s, 3H). LC-MS m/z: 380.2 [M+H]⁺. HPLC Purity (214 nm): 97.14%; t_{R}=7.17 min.

### Example 258—N-(2-(3,3-Difluorocyclopentyl)ethyl)-4-(4-methylpiperazin-1-yl)-1H-benzo[d]imidazole-1-carboxamide

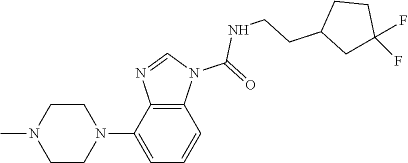 [SMILES: CN1CCN(c2cccc3c2ncn3C(=O)NCCC2CCC(F)(F)C2)CC1]

Following general procedure C, 4-(4-methylpiperazin-1-yl)-1H-benzo[d]imidazole (200 mg, 1.92 mmol) and 2-(3,3-difluorocyclopentyl)ethanamine (70 mg, 0.46 mmol) afforded the title compound (130.2 mg, 71.2%) as a white solid. ¹H NMR (400 MHz, CDCl₃) δ 8.45 (s, 1H), 7.40 (d, J=8.1 Hz, 1H), 7.26 (d, J=9.2 Hz, 1H), 6.71 (d, J=7.9 Hz, 1H), 6.40 (t, J=5.3 Hz, 1H), 3.59 (bs, 4H), 3.52-3.47 (m, 2H), 2.86 (bs, 4H), 2.47 (s, 3H), 2.43-1.96 (m, 6H), 1.79 (dd, J=14.7, 7.2 Hz, 2H), 1.47 (td, J=9.2, 2.6 Hz, 1H). LC-MS m/z: 391.46 [M+H]⁺. HPLC Purity (214 nm): 95.78%; t_{R}=6.04 min.

### Example 259—N-(2-(2,2-Difluorocyclopentyl)ethyl)-4-(4-methylpiperazin-1-yl)-1H-benzo[d]imidazole-1-carboxamide

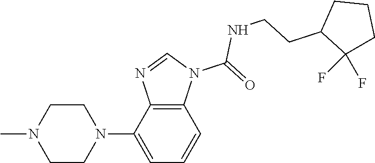 [SMILES: CN1CCN(c2cccc3c2ncn3C(=O)NCCC2CCCC2(F)F)CC1]

Following general procedure C, 4-(4-methylpiperazin-1-yl)-1H-benzo[d]imidazole (200 mg, 1.92 mmol) and 2-(2,2-difluorocyclopentyl)ethanamine (120 mg, 0.81 mmol) afforded the title compound (17 mg, 4.7%) as a white solid. ¹H NMR (400 MHz, MeOD) δ 8.57 (s, 2H), 7.76 (d, J=8.0 Hz, 1H), 7.33 (t, J=8.1 Hz, 1H), 6.87 (d, J=7.8 Hz, 1H), 3.65 (bs, 4H), 3.51 (t, J=7.5 Hz, 2H), 3.41-3.32 (m, 4H), 2.87 (s, 3H), 2.26-1.96 (m, 5H), 1.85-1.67 (m, 3H), 1.56-1.48 (m, 1H). LC-MS m/z: 391.46 [M+H]⁺. HPLC Purity (214 nm): 100%; t_{R}=6.09 min.

### Example 260—N-(3-(3,3-Difluorocyclobutyl)propyl)-4-(4-methylpiperazin-1-yl)-1H-benzo[d]imidazole-1-carboxamide

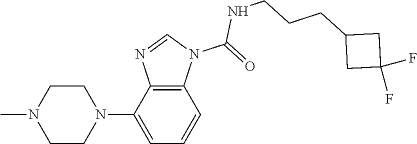 [SMILES: CN1CCN(c2cccc3c2ncn3C(=O)NCCCC2CC(F)(F)C2)CC1]

Following general procedure C, 4-(4-methylpiperazin-1-yl)-1H-benzo[d]imidazole (100 mg, 0.46 mmol) and 3-(3,3-difluorocyclobutyl)propan-1-amine (70 mg, 0.37 mmol) afforded the title compound (5.7 mg, 3.0%) as a colorless oil. ¹H NMR (400 MHz, CDCl₃) δ 8.46 (s, 1H), 7.44 (d, J=8.3 Hz, 1H), 7.30 (d, J=7.9 Hz, 1H), 6.74 (d, J=8.0 Hz, 1H), 6.47 (bs, 1H), 3.70 (bs, 4H), 3.50 (t, J=6.1 Hz, 2H), 3.13 (bs, 4H), 2.69 (s, 1H), 2.64 (s, 3H), 2.22-2.13 (m, 4H), 1.64 (bs, 4H). LC-MS m/z: 392.3 [M+H]⁺. HPLC Purity (214 nm): 95.02%; t_{R}=5.33 min.

### Example 261—4-(1-Ethylpyrrolidin-3-yl)-N-(3-phenylpropyl)-1H-benzo[d]imidazole-1-carboxamide

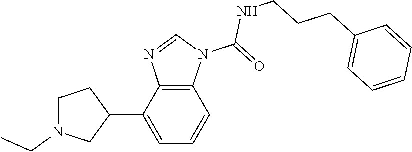 [SMILES: CCN1CCC(c2cccc3c2ncn3C(=O)NCCCc2ccccc2)C1]

Following general procedure A, 4-bromo-1H-benzo[d]imidazole (594 mg, 3.0 mmol) and tert-butyl 3-(4,4,5,5-tetramethyl-1,3,2-dioxaborolan-2-yl)-2,5-dihydro-1H-pyrrole-1-carboxylate (974 mg, 3.3 mmol) afforded tert-butyl 3-(1H-benzo[d]imidazol-4-yl)-2,5-dihydro-1H-pyrrole-1-carboxylate (590 mg, 69%) as a yellow oil. LC-MS m/z: 230.0 [M-55]⁺. Purity (214 nm): 77%; t_{R}=1.61 min.

To a solution of tert-butyl 3-(1H-benzo[d]imidazol-4-yl)-2,5-dihydro-1H-pyrrole-1-carboxylate (590 mg, 2.0 mmol) in DCM (4 mL) was added HCl/dioxane (5 mL, 20 mmol). The reaction mixture was stirred at RT for 2 h and concentrated to give crude 4-(2,5-dihydro-1H-pyrrol-3-yl)-1H-benzo[d]imidazole (510 mg) as a yellow solid. LC-MS m/z: 186.2 [M+H]⁺. Purity (214 nm): 81%; t_{R}=1.42 min.

A mixture of 4-(2,5-dihydro-1H-pyrrol-3-yl)-1H-benzo[d]imidazole (370 mg, 2.0 mmol), CH₃CHO (123.5 mg, 2.8 mmol), NaBH₃CN (256 mg, 4.0 mmol) and MeOH (10 mL) was stirred at RT for 2 h. Then cooled, concentrated and purified by silica gel column chromatography (DCM:MeOH=20:1) to give 4-(1-ethylpyrrolidin-3-yl)-1H-benzo[d]imidazole (150 mg, 35%) as a yellow oil. LC-MS m/z: 216.2 [M+H]⁺. Purity (214 nm): 89%; t_{R}=1.19 min.

Following general procedure C, 4-(1-ethylpyrrolidin-3-yl)-1H-benzo[d]imidazole (129 mg, 0.6 mmol) and 3-phenylpropan-1-amine (81 mg, 0.6 mmol) afforded the title compound (47.8 mg, 18%) as a white solid. ¹H NMR (400 MHz, CDCl₃) δ 8.60 (s, 1H), 8.31 (s, 1H), 7.90 (d, J=7.2 Hz, 1H), 7.31-7.27 (m, 3H), 7.26-7.19 (m, 3H), 7.06 (d, J=6.4 Hz, 1H), 3.94 (bs, 1H), 3.70 (bs, 1H), 3.51-3.46 (m, 5H), 3.11 (s, 2H), 2.77-2.74 (m, 2H), 2.45-2.35 (m, 2H), 2.06-2.04 (m, 2H), 1.31 (s, 3H). LC-MS m/z: 377.3 [M+H]⁺. HPLC Purity (214 nm): 100%, t_{R}=6.49 min.

### Example 262—4-(3-(Azetidin-1-yl)propyl)-N-(3-phenylpropyl)-1H-benzo[d]imidazole-1-carboxamide

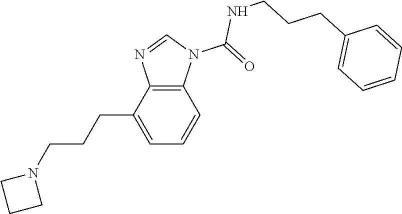 [SMILES: O=C(NCCCc1ccccc1)n1cnc2c(CCCN3CCC3)cccc21]

To a solution of 1H-benzo[d]imidazole-4-carbaldehyde (1.0 g, 6.9 mmol) and ethyl 2-(diethoxyphosphoryl) acetate (2.3 g, 10.3 mmol) in THF (10 mL) was added NaH (0.55 g, 13.70 mmol) at 0° C. and the mixture was stirred at RT under N₂ for 2 h. The mixture was quenched with H₂O (10 mL) and extracted with DCM (10 mL×3). The combined organic layers were washed with brine (20 mL), dried over Na₂SO₄, filtered, concentrated and purified by silica gel column chromatography (DCM:MeOH=10:1) to give ethyl (E)-3-(1H-benzo[d]imidazol-4-yl)acrylate (1.3 g, 88%) as a yellow solid. LC-MS m/z: 217.1 [M+H]⁺. Purity (214 nm): 99%; t_{R}=1.73 min.

To a solution of ethyl (E)-3-(1H-benzo[d]imidazol-4-yl)acrylate (1.3 g, 4.6 mmol) in MeOH (15 mL) was added Pd/C (200 mg) and the mixture was stirred at RT under H2 for 16 h. The mixture was filtered and concentrated to give ethyl 3-(1H-benzo[d]imidazol-4-yl)propanoate (1.0 g, 76%) as a white solid. LC-MS m/z: 219.2 [M+H]⁺. Purity (214 nm): 99%; t_{R}=1.66 min.

To a solution of ethyl 3-(1H-benzo[d]imidazol-4-yl)propanoate (1.0 g, 4.6 mmol) in THF (5 mL) was added LAH (0.35 g, 9.2 mmol) at 0° C. and the reaction mixture was stirred at RT under N₂ for 2 h. The mixture was quenched with water (10 mL), dried over Na₂SO₄, filtered and concentrated to give 3-(1H-benzo[d]imidazol-4-yl)propan-1-ol (600 mg, 75%) as a white solid. LC-MS m/z: 174.0 [M+H]⁺. Purity (214 nm): 99%; t_{R}=1.4 min.

To a solution of 3-(1H-benzo[d]imidazol-4-yl)propan-1-ol (600 mg, 3.4 mmol) in DCM (8 mL) was added Et₃N (689 mg, 6.8 mmol) and MsCl (428 mg, 3.8 mmol). The mixture was stirred at RT under N₂ for 2 h, concentrated and purified by silica gel column chromatography (DCM:MeOH=10:1) to give 3-(1H-benzo[d]imidazol-4-yl)propyl methanesulfonate (1.0 g, 100%) as a yellow oil. LC-MS m/z: 333.0 [M+H]⁺. Purity (214 nm): 44%; t_{R}=1.16 min.

Following general procedure E, 3-(1H-benzo[d]imidazol-4-yl)propyl methanesulfonate (900 mg, 2.7 mmol) and azetidine (618 mg, 10.8 mmol) afforded 4-(3-(azetidin-1-yl)propyl)-1H-benzo[d]imidazole (300 mg, 39%) as a yellow oil. LC-MS m/z: 216.0 [M+H]⁺. Purity (214 nm): 95%; t_{R}=1.16 min.

Following general procedure C, 4-(3-(azetidin-1-yl)propyl)-1H-benzo[d]imidazole (300 mg, 1.4 mmol) and 3-phenylpropan-1-amine (188.5 mg, 2.8 mmol) afforded the title compound (27.9 mg, 7.4%) as a yellow solid. ¹H NMR (400 MHz, CDCl₃) δ 8.57 (s, 1H), 8.12 (bs, 1H), 7.86 (d, J=8.3 Hz, 1H), 7.28-7.26 (m, 2H), 7.24-7.12 (m, 4H), 7.04 (d, J=8.3 Hz, 1H), 3.80 (bs, 4H), 3.50-3.47 (m, 2H), 3.02 (bs, 2H), 2.89 (bs, 2H), 2.74 (t, J=7.6 Hz, 2H), 2.32 (bs, 2H), 2.07-2.01 (m, 4H). LC-MS m/z: 377.2 [M+H]⁺. HPLC Purity (214 nm): 100%; t_{R}=5.54 min.

### Example 263—4-(4-Amino-4-methylpiperidin-1-yl)-N-(3-phenylpropyl)-1H-benzo[d]imidazole-1-carboxamide

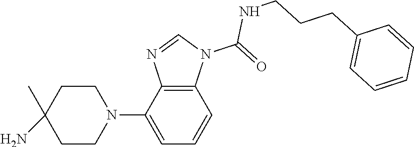 [SMILES: CC1(N)CCN(c2cccc3c2ncn3C(=O)NCCCc2ccccc2)CC1]

Following general procedure E, tert-butyl 4-methylpiperidin-4-ylcarbamate (1.0 g, 4.72 mmol) and 3-fluoro-2-nitroaniline (729 mg, 4.67 mmol) afforded tert-butyl (1-(3-amino-2-nitrophenyl)-4-methylpiperidin-4-yl)carbamate (1.8 g, 100%) as a red oil. LC-MS m/z: 351.3 [M+H]⁺. Purity (214 nm): 92.34%; t_{R}=2.06 min.

Following general procedure F, tert-butyl (1-(3-amino-2-nitrophenyl)-4-methyl piperidin-4-yl)carbamate (1.8 g, 5.14 mmol) afforded tert-butyl(1-(2,3-diaminophenyl)-4-methylpiperidin-4-yl)carbamate (1.7 g, 100%) as a brown oil. LC-MS m/z: 321.6 [M+H]⁺. Purity (254 nm): 94.96%; t_{R}=1.93 min.

Following general procedure G (method B), tert-butyl (1-(2,3-diaminophenyl)-4-methylpiperidin-4-yl)carbamate (1.7 g, 5.31 mmol) afforded 1-(1H-benzo[d]imidazol-4-yl)-4-methylpiperidin-4-amine (500 mg, 41.7%) as a brown oil. LC-MS m/z: 234.2 [M+H]⁺. Purity (254 nm): 97.06%; t_{R}=1.34 min.

Following general procedure C, 1-(1H-benzo[d]imidazol-4-yl)-4-methylpiperidin-4-amine (500 mg, 2.17 mmol) and 3-phenylpropan-1-amine (294 mg, 2.17 mmol) afforded the title compound (115.7 mg, 13.6%) as a white solid. ¹H NMR (500 MHz, CDCl₃) δ 8.55 (brs, 1H), 8.48 (s, 1H), 7.71 (dd, J=8.0, 1.5 Hz, 1H), 7.32-7.24 (m, 4H), 7.20-7.14 (m, 2H), 6.87 (d, J=7.2 Hz, 1H), 3.80-3.77 (m, 2H), 3.46 (t, J=7.2 Hz, 2H), 3.38-3.32 (m, 2H), 3.26-3.18 (m, 2H), 2.76 (t, J=7.6 Hz, 2H), 2.12-1.95 (m, 6H), 1.49 (s, 3H). LC-MS m/z: 392.1 [M+H]⁺. HPLC Purity (214 nm): 100%; t_{R}=6.33 min.

### Example 264—4-(3-(Dimethylamino)pyrrolidin-1-yl)-N-(3-phenylpropyl)-1H-benzo[d]imidazole-1-carboxamide

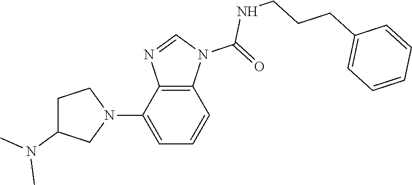 [SMILES: CN(C)C1CCN(c2cccc3c2ncn3C(=O)NCCCc2ccccc2)C1]

Following general procedure E, N. N-dimethylpyrrolidin-3-amine hydrochloride (1.2 g, 10.5 mmol) and 3-fluoro-2-nitroaniline (1.6 g, 10.5 mmol) afforded 1-(3-amino-2-nitrophenyl)-N,N-dimethylpyrrolidin-3-amine (524 mg, 20.15%) as a red solid. LC-MS m/z: 251.1 [M+H]⁺. Purity (214 nm): 100%; t_{R}=0.36 min.

Following general procedure F, 1-(3-amino-2-nitrophenyl)-N, N-dimethylpyrrolidin-3-amine (524 mg, 2.1 mmol) afforded 3-(3-(dimethylamino)pyrrolidin-1-yl)benzene-1,2-diamine (404 mg, 87.6%) as a red solid. LC-MS m/z: 221.2 [M+H]⁺. Purity (214 nm): 98.52%; t_{R}=1.23 min.

Following general procedure G (method B), 3-(3-(dimethylamino)pyrrolidin-1-yl) benzene-1,2-diamine (404 mg, 1.48 mmol) afforded 1-(1H-benzo[d]imidazol-4-yl)-N, N-dimethyl pyrrolidin-3-amine (220 mg, 52.1%) as a red oil. LC-MS m/z: 231.2 [M+H]⁺. Purity (254 nm): 98.58%; t_{R}=1.54 min.

Following general procedure C, 1-(1H-benzo[d]imidazol-4-yl)-N, N-dimethylpyrrolidin-3-amine (220 mg, 0.95 mmol) and 3-phenylpropan-1-amine (135 mg, 1 mmol) afforded the title compound (144.0 mg, 38.5%) as a yellow solid. ¹H NMR (400 MHz, CDCl₃) δ 8.18 (s, 1H), 7.30 (dd, J=10.0, 4.8 Hz, 2H), 7.26-7.20 (m, 4H), 6.91 (d, J=8.0 Hz, 1H), 6.37 (d, J=8.0 Hz, 1H), 5.79 (t, J=5.6 Hz, 1H), 4.07-3.90 (m, 2H), 3.77 (td, J=9.8, 7.2 Hz, 1H), 3.67 (t, J=8.8 Hz, 1H), 3.54 (dd, J=13.0, 6.9 Hz, 2H), 3.01 (bs, 1H), 2.77 (t, J=7.5 Hz, 2H), 2.42 (s, 6H), 2.27 (dt, J=11.8, 5.6 Hz, 1H), 2.16-1.88 (m, 4H). LC-MS m/z: 392.3 [M+H]⁺. HPLC Purity (214 nm): 100%; t_{R}=6.50 min.

### Example 265—4-(Hexahydropyrrolo[3,4-c]pyrrole-2(1H)-yl)-N-(3-phenylpropyl)-1H-benzo[d]imidazole-1-carboxamide

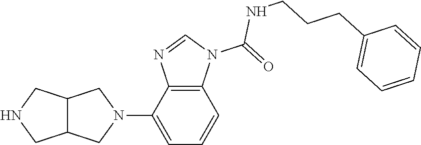 [SMILES: O=C(NCCCc1ccccc1)n1cnc2c(N3CC4CNCC4C3)cccc21]

Following general procedure E, 3-fluoro-2-nitroaniline (500 mg, 3.21 mmol) and tert-butyl hexahydropyrrolo[3,4-c]pyrrole-2(1H)-carboxylate (1.0 g, 4.81 mmol) afforded tert-butyl 5-(3-amino-2-nitrophenyl)hexahydropyrrolo[3,4-c]pyrrole-2(1H)-carboxylate (1.0 g, 90%) as an orange solid. LC-MS m/z: 349.0 [M+H]⁺. Purity (214 nm): 96%; t_{R}=2.01 min.

Following general procedure F, tert-butyl 5-(3-amino-2-nitrophenyl)hexahydropyrrolo[3,4-c]pyrrole-2(1H)-carboxylate (1.0 g, 2.87 mmol) afforded tert-butyl 5-(2,3 diaminophenyl)hexahydropyrrolo[3,4-c]-pyrrole-2(1H)-carboxylate (900 mg, 98%) as a brown oil. LC-MS m/z: 319.2 [M+H]⁺. Purity (214 nm): 91%; t_{R}=1.75 min.

Following general procedure G (method B), tert-butyl 5-(2,3 diaminophenyl) hexahydropyrrolo[3,4-c]-pyrrole-2(1H)-carboxylate (1.0 g, 3.14 mmol) afforded 4-(hexahydropyrrolo[3,4-c]pyrrol-2(1H)-yl)-1H-benzo[d]-imidazole (400 mg, 56%) as a white solid. LC-MS m/z: 229.2 [M+H]⁺. Purity (214 nm): 99%; t_{R}=1.12 min.

To a solution of 4-(hexahydropyrrolo[3,4-c]pyrrol-2(1H)-yl)-1H-benzo[d]imidazole (390 mg, 1.71 mmol) in MeOH (3 mL) was added benzaldehyde (181 mg, 1.71 mmol) at RT and the mixture was stirred for 30 min followed by the addition of NaBH₃CN (323 mg, 5.13 mmol). The resulting mixture was stirred at RT for 16 h, concentrated and purified by silica gel column chromatography (MeOH:DCM=1:9) to give 4-(5-benzylhexahydropyrrolo[3,4-c]pyrrol-2(1H)-yl)-1H-benzo[d]imidazole (500 mg, 92%) as a clear oil. LC-MS m/z: 319.2 [M+H]⁺. Purity (214 nm): 98%; t_{R}=1.69 min.

Following general procedure C, 4-(5-benzylhexahydropyrrolo[3,4-c]pyrrol-2(1H)-yl)-1H-benzo[d]imidazole (300 mg, 0.94 mmol) and 3-phenylpropan-1-amine (191 mg, 1.42 mmol) afforded 4-(5-benzylhexahydropyrrolo[3,4-c]pyrrol-2(1H)-yl)-N-(3-phenylpropyl)-1H-benzo[d]imidazole-1-carboxamide (130 mg, 29%) as a white solid. LC-MS m/z: 480.0 [M+H]⁺. Purity (214 nm): 98%; t_{R}=1.74 min.

To a solution of 4-(5-benzylhexahydropyrrolo[3,4-c]pyrrol-2(1H)-yl)-N-(3-phenylpropyl)-1H-benzo[d]imidazole-1-carboxamide (100 mg, 0.21 mmol) in MeOH (30 mL) was added Pd/C (40 mg) and (Boc)₂O (46 mg, 0.21 mmol) at RT and the mixture was stirred under an atmospheric pressure of H₂ for 16 h. The mixture was filtered, concentrated and purified by silica gel column chromatography (EA:PE=1:1) to give tert-butyl 5-(1-((3-phenylpropyl)carbamoyl)-1H-benzo[d]imidazol-4-yl)-hexahydropyrrolo[3,4-c]pyrrole-2(11H)-carboxylate (52 mg, 51%) as a clear oil. LC-MS m/z: 490.1 [M+H]⁺. Purity (214 nm): 99%; t_{R}=1.60 min.

A solution of tert-butyl 5-(1-((3-phenylpropyl)carbamoyl)-1H-benzo[d]imidazol-4-yl)hexahydropyrrolo[3,4-c]pyrrole-2(1H)-carboxylate (45 mg, 0.09 mmol) in HCOOH/DCM (1/1, 2 mL) was stirred at RT for 16 h. The reaction mixture was concentrated and purified by Prep-HPLC to give the title compound (16.5 mg, 45%) as a white solid. ¹H NMR (400 MHz, DMSO-d₆) δ 8.60 (s, 1H), 8.37 (s, 1H), 7.43 (d, J=7.9 Hz, 1H), 7.34-7.22 (m, 4H), 7.21-7.09 (m, 2H), 6.43 (d, J=7.7 Hz, 1H), 3.65 (bs, 3H), 3.36-3.28 (m, 3H), 3.26 (bs, 2H), 2.96 (bs, 2H), 2.87 (d, J=20=9.7 Hz, 2H), 2.68 (t, J=7.2 Hz, 2H), 1.89 (dt, J=13.9, 6.7 Hz, 2H). LC-MS m/z: 390.1 [M+H]⁺. HPLC Purity (214 nm): 100%; t_{R}=6.46 min.

### Example 266—4-(5-Methylhexahydropyrrolo[3,4-c]pyrrol-2(1H)-yl)-N-(3-phenylpropyl)-1H-benzo[d]imidazole-1-carboxamide

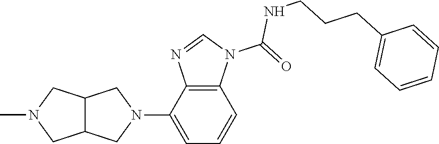 [SMILES: CN1CC2CN(c3cccc4c3ncn4C(=O)NCCCc3ccccc3)CC2C1]

To a solution of tert-butyl hexahydropyrrolo[3,4-c]pyrrole-2(1H)-carboxylate (3.0 g, 14.15 mmol) in THF (10 mL) was added LAH (28 mL, 70.75 mmol) at 0° C. and the mixture was stirred at RT for 8 h. Then Na₂SO₄10H2O (5.0 g) was added and the mixture was stirred for 30 min, filtered and concentrated to give 2-methyloctahydropyrrolo[3,4-c]pyrrole (1.02 g, 57.3%) as a yellow oil. LC-MS m/z: 127.7 [M+H]⁺, t_{R}=0.36 min.

Following general procedure E, 2-methyloctahydropyrrolo[3,4-c]pyrrole (1.02 g, 8.10 mmol) and 3-fluoro-2-nitroaniline (842.4 mg, 5.4 mmol) afforded 3-(5-methylhexahydropyrrolo[3,4-c]pyrrol-2(1H)-yl)-2-nitroaniline (730 mg, 51.6%) as a yellow oil. LC-MS m/z: 263.1 [M+H]⁺. Purity (214 nm): 80.13%; t_{R}=0.53 min.

Following general procedure F, 3-(5-methylhexahydropyrrolo[3,4-c]pyrrol-2(1H)-yl)-2-nitroaniline (730 mg, 2.79 mmol) afforded crude 3-(5-methylhexahydropyrrolo[3,4-c]pyrrol-2(1H)-yl)benzene-1,2-diamine (630 mg, 97.4%) as a yellow oil which was used directly in the next step. LC-MS m/z: 233.7 [M+H]⁺. Purity (214 nm): 85%; t_{R}=0.18 min.

Following general procedure G (method B), 3-(5-methylhexahydropyrrolo[3,4-c]pyrrol-2(1H)-yl)benzene-1,2-diamine (630 mg, 2.72 mmol) afforded crude 4-(5-methylhexahydropyrrolo[3,4-c]-pyrrol-2(1H)-yl)-1H-benzo[d]imidazole (600 mg, 91.5%) as a yellow oil which was used directly in the next step. LC-MS m/z: 243.1 [M+H]⁺. Purity (214 nm): 88%; t_{R}=1.45 min.

Following general procedure C. 4-(5-methylhexahydropyrrolo[3,4-c]pyrrol-2(1H)-yl)-1H-benzo[d]imidazole (121 mg, 0.5 mmol) and 3-phenylpropan-1-amine (68 mg, 0.5 mmol) afforded the title compound (31.5 mg, 15.6%) as a white solid. ¹H NMR (400 MHz, CDCl₃) δ 8.33 (s, 1H), 7.30 (t, J=7.9 Hz, 3H), 7.25-7.17 (m, 4H), 6.76 (bs, 1H), 6.54 (d, J=7.5 Hz, 1H), 4.05 (d, J=10.1 Hz, 2H), 3.72 (bs, 2H), 3.54 (q, J=5.9 Hz, 2H), 3.30-3.19 (m, 4H), 2.77 (t, J=7.5 Hz, 2H), 2.71 (dd, J=10.4, 7.1 Hz, 2H), 2.67 (s, 3H), 2.05 (p, J=7.6 Hz, 2H). LC-MS m/z: 404.7 [M+H]⁺. HPLC Purity (214 nm): 99.90%, t_{R}=6.55 min.

### Example 267—4-(1-Methylpyrrolidin-3-yl)-N-(3-phenylpropyl)-1H-benzo[d]imidazole-1-carboxamide

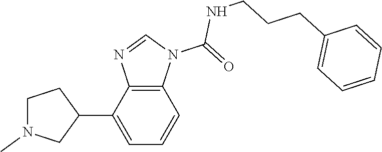 [SMILES: CN1CCC(c2cccc3c2ncn3C(=O)NCCCc2ccccc2)C1]

Following general procedure A, 4-bromo-1H-benzo[d]imidazole (1.0 g, 5.1 mmol) and tert-butyl 3-(4,4,5,5-tetramethyl-1,3,2-dioxaborolan-2-yl)-2,5-dihydro-1H-pyrrole-1-carboxylate (1.8 g, 6.12 mmol) afforded tert-butyl 3-(1H benzo[d]imidazol-4-yl)-2,5-dihydro-1H-pyrrole-1-carboxylate (1.31 g, 90.3%) as a black oil. LC-MS m/z: 286.1 [M+H]⁺. Purity (214 nm): 82.97%; t_{R}=1.90 mins.

A solution of tert-butyl 3-(1H-benzo[d]imidazol-4-yl)-2,5-dihydro-1H-pyrrole-1-carboxylate (1.31 g, 4.6 mmol) in HCl-dioxane (10 ml) was stirred at RT for 3 h. The mixture was concentrated to give 4-(2,5-dihydro-1H-pyrrol-3-yl)-1H-benzo[d]imidazole (1.02 g, 119.8%) as a black solid which was used directly in the next reaction. LC-MS m/z: 186.1. [M+H]⁺. Purity (214 nm): 68%; t_{R}=0.36 min.

To a solution of 4-(2,5-dihydro-1H-pyrrol-3-yl)-1H-benzo[d]imidazole (700 mg, 3.78 mmol) in MeOH (10 mL) was added HCHO (1.3 g, 11.34 mol) and the mixture was stirred at RT for 1 h. NaBH₃CN (475 mg, 7.57 mmol) was then added and the reaction mixture was stirred for another 1 h. The mixture was concentrated and purified by silica gel column chromatography (DCM/MeOH=10:3) to give 4-(1-methyl-2,5-dihydro-1H-pyrrol-3-yl)-1H-benzo[d]imidazole (658 mg, 87.4%) as a yellow solid. LC-MS m/z: 200.1 [M+H]⁺, t_{R}=0.19 min.

To a solution of 4-(1-methyl-2,5-dihydro-1H-pyrrol-3-yl)-1H-benzo[d]imidazole (658 mg, 3.3 mmol) in MeOH (100 mL) was added PtO₂ (30%, 200 mg) and the mixture was stirred at RT 15 h under H₂. The mixture was filtered and concentrated to give crude 4-(1-methylpyrrolidin-3-yl)-1H-benzo[d]imidazole (642 mg, 96.8%) as a white solid. The crude was used into next step reaction directly. LC-MS m/z: 202.1 [M+H]⁺, t_{R}=1.14 min.

Following general procedure C, 4-(1-methylpyrrolidin-3-yl)-1H-benzo-[d]imidazole (70 mg, 0.35 mmol) and 3-phenylpropan-1-amine (47 mg, 0.35 mmol) afforded the title compound (22.1 mg, 17.4%) as a white solid. ¹H NMR (400 MHz, CDCl₃) δ 8.63 (s, 1H), 8.09 (s, 1H), 7.89 (d, J=8.2 Hz, 1H), 7.30-7.18 (m, 6H), 7.08 (d, J=7.2 Hz, 1H), 4.03-3.90 (m, 1H), 3.68 (bs, 1H), 3.55-3.40 (m, 5H), 2.83 (s, 3H), 2.76 (t, J=7.4 Hz, 2H), 2.49-2.39 (m, 2H), 2.05 (p, J=7.4 Hz, 2H). LC-MS m/z: 363.1 [M+H]⁺. HPLC Purity (214 nm): 96.39%; t_{R}=6.35 min.

### Example 268—4-(3-(Methylamino)pyrrolidin-1-yl)-N-(3-phenylpropyl)-1H-benzo[d]imidazole-1-carboxamide

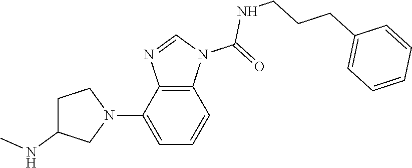 [SMILES: CNC1CCN(c2cccc3c2ncn3C(=O)NCCCc2ccccc2)C1]

Following general procedure E, N-methylpyrrolidin-3-amine (800 mg, 8 mmol) and 3-fluoro-2-nitroaniline (1.0 g, 6.4 mmol) afforded 1-(3-amino-2-nitrophenyl)-N-methylpyrrolidin-3-amine (400 mg, 21.2%) as a red solid. LC-MS m/z: 237.2 [M+H]⁺. Purity (254 nm): 95.16%; t_{R}=1.31 min.

Following general procedure F, 1-(3-amino-2-nitrophenyl)-N-methylpyrrolidin-3-amine (400 mg, 1.7 mmol) afforded 3-(3-(methylamino)pyrrolidin-1-yl)benzene-1,2-diamine (350 mg, 99.9%) as a red oil. LC-MS m/z: 207.2 [M+H]⁺. Purity (254 nm): 91.63%; t_{R}=0.96 min.

Following general procedure G (method B), 3-(3-(methylamino)pyrrolidin-1-yl)benzene-1,2-diamine (350 mg, 1.7 mmol) afforded 1-(1H-benzo[d]imidazol-4-yl)-N-methylpyrrolidin-3-amine (330 mg, 92.3%) as a red oil. LC-MS m/z: 217.2 [M+H]⁺. Purity (254 nm): 88.72%/0; t_{R}=1.16 min.

Following general procedure C, 1-(1H-benzo[d]imidazol-4-yl)-N-methylpyrrolidin-3-amine (330 mg, 1.4 mmol) and 3-phenylpropan-1-amine (189 mg, 1.4 mmol) afforded the title compound (13.4 mg, 48.8%) as a white solid. ¹H NMR (400 MHz, DMSO-d₆) δ 8.51 (bs, 1H), 8.46 (s, 1H), 7.42-7.12 (m, 7H), 6.35 (d, J=8.0 Hz, 1H), 3.92 (dd, J=6.2, 2.4 Hz, 2H), 3.77-3.61 (m, 4H), 3.32 (dd, J=12.4, 6.6 Hz, 2H), 2.69 (t, J=7.6 Hz, 2H), 2.39 (s, 3H), 2.14 (bs, 1H), 1.91-1.83 (m, 3H). LC-MS m/z: 378.0 [M+H]⁺. HPLC Purity (254 nm): 100%; t_{R}=6.44 min.

### Example 269—4-(4-Methyl-1,4-diazepan-1-yl)-N-(3-phenylpropyl)-1H-benzo[a]imidazole-1-carboxamide

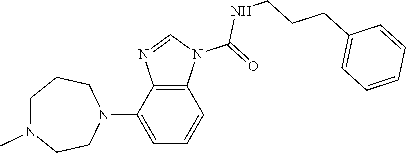 [SMILES: CN1CCCN(c2cccc3c2ncn3C(=O)NCCCc2ccccc2)CC1]

Following general procedure E, 3-fluoro-2-nitroaniline (390 mg, 2.49 mmol) and 1-methyl-1,4-diazepane (450 mg, 2.49 mmol) afforded 3-(4-methyl-1,4-diazepan-1-yl)-2-nitroaniline (270 mg, 43%) as an orange solid. LC-MS m/z: 251.2 [M+H]⁺. Purity (214 nm): >96%0; t_{R}=1.38 min.

Following general procedure F, 3-(4-methyl-1,4-diazepan-1-yl)-2-nitroaniline (270 mg, 1.08 mmol) afforded 3-(4-methyl-1,4-diazepan-1-yl)benzene-1,2-diamine (240 mg, 98%) as a pale oil. LC-MS m/z: 221.2 [M+H]⁺, t_{R}=1.10 min.

Following general procedure G (method B), 3-(4-methyl-1,4-diazepan-1-yl)benzene-1,2-diamine (210 mg, 0.95 mmol) afforded 4-(4-methyl-1,4-diazepan-1-yl)-1H-benzo[d]imidazole (200 mg, 92%) as a colorless oil. LC-MS m/z: 231.2 [M+H]⁺. Purity (214 nm): >95%; t_{R}=1.38 min.

Following general procedure C, 4-(4-methyl-1,4-diazepan-1-yl)-1H-benzo[d]imidazole (150 mg, 0.65 mmol) and 3-phenylpropan-1-amine (88 mg, 0.65 mmol) afforded the title compound (32.0 mg, 7.4%) as a yellow solid. ¹H NMR (400 MHz, CDCl₃) δ 8.46 (s, 1H), 8.06 (bs, 1H), 7.46 (d, J=8.1 Hz, 1H), 7.33-7.27 (m, 2H), 7.26-7.17 (m, 4H), 6.49 (d, J=8.1 Hz, 1H), 4.09 (bs, 1H), 3.61-3.52 (m, 6H), 3.23 (bs, 2H), 2.80-2.75 (m, 5H), 2.41 (bs, 2H), 2.06 (p, J=7.6 Hz, 2H). LC-MS m/z: 392.1 [M+H]⁺. HPLC Purity (214 nm): 100%; t_{R}=9.13 min.

### Example 270—4-(1-Methylazetidin-3-yl)-N-(3-phenylpropyl)-1H-benzo[d]imidazole-1-carboxamide

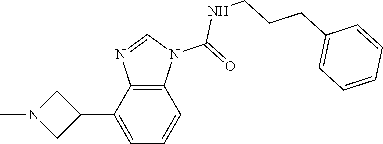 [SMILES: CN1CC(c2cccc3c2ncn3C(=O)NCCCc2ccccc2)C1]

To a solution of 4-bromo-1-((2-(trimethylsilyl)ethoxy)methyl)-1H-benzo[d]imidazole (7.6 g, 23.3 mmol) in dioxane (100 mL) was added KOAc (6.9 g, 69.9 mmol) and PinBBPin (8.9 g, 35.0 mmol). The reaction mixture was stirred at 110° C. under N₂ (1 atm) for 30 h. The reaction mixture was cooled, filtered, concentrated and purified by silica gel column chromatography (DCM/MeOH=1/1) to give 1-((2-(trimethylsilyl)ethoxy)methyl)-1H-benzo[d]imidazol-4-ylboronic acid (6.0 g, 88.2%) as a blank oil. LC-MS m/z: 293.2 [M+H]⁺. Purity (254 nm): 100%; t_{R}=1.48 min.

To a solution of 1-((2-(trimethylsilyl)ethoxy)methyl)-1H-benzo[d]imidazol-4-ylboronic acid (6.0 g, 20.5 mmol) in dioxane (150 mL) was added tert-butyl 3-(2-tosylhydrazono)azetidine-1-carboxylate (10.4 g, 38.0 mmol) and Cs₂CO₃ (10.0 g, 30.8 mmol) and the reaction mixture was stirred at 110° C. for 16 h. The reaction mixture was cooled, filtered, concentrated and purified by silica gel column chromatography (PE/EA=1/1) to give tert-butyl 3-(1-((2-(trimethylsilyl)ethoxy)-methyl)-1H-benzo[d]imidazol-4-yl)azetidine-1-carboxylate (1.2 g, 14.46%) as a dark solid. LC-MS m/z: 404.1 [M+H]⁺. Purity (254 mnm): 60.81%; t_{R}=2.05 min.

A solution of tert-butyl 3-(1-((2-(trimethylsilyl)ethoxy)methyl)-1H-benzo[d]imidazol-4-yl)azetidine-1-carboxylate (1.2 g, 2.9 mmol) in TFA (10 mL) was stirred at RT for 3 h and then concentrated to give crude 4-(azetidin-3-yl)-1H-benzo[d]imidazole (1.2 g) as a red solid. LC-MS m/z: 174.1 [M+H]⁺. Purity (254 nm): 86.44%; t_{R}=0.67 min.

A solution of 4-(azetidin-3-yl)-1H-benzo[d]imidazole (600 mg, 3.5 mmol) and HCHO (475 mg, 4.2 mmol) in MeOH (30 mL) was stirred at RT for 1 h and then NaBH₃CN (650 mg, 10.5 mmol) was added at 0° C. The reaction mixture was stirred at RT for 16 h, concentrated and purified by silica gel column chromatography (DCM/MeOH=10/1) to give 4-(1-methylazetidin-3-yl)-1H-benzo[d]imidazole (120 mg, 20.7% over two steps) as a yellow oil. LC-MS m/z: 188.1 [M+H]⁺. Purity (254 nm): 96.11%; t_{R}=1.21 min.

Following general procedure C, 4-(1-methylazetidin-3-yl)-1H-benzo[d]imidazole (120 mg, 0.6 mmol) and 3-phenylpropan-1-amine (81 mg, 0.6 mmol) afforded the title compound (30.5 mg, 13.7%) as a yellow solid. ¹H NMR (500 MHz, CDCl₃) δ 8.81 (bs, 1H), 8.76 (s, 1H), 8.06 (d, J=8.1 Hz, 1H), 7.32-7.28 (m, 2H), 7.28-7.15 (m, 4H), 7.00 (d, J=7.4 Hz, 1H), 4.93-4.19 (m, 4H), 4.36-4.06 (m, 1H), 3.55 (dd, J=12.6, 6.8 Hz, 2H), 2.96 (s, 3H), 2.78 (d, J=7.8 Hz, 2H), 2.08 (p, J=7.8 Hz, 2H). LC-MS m/z: 349.4 [M+H]⁺. HPLC Purity (254 nm): 100%; t_{R}=5.74 min.

### Example 271—4-(1-Methyloctahydro-6H-pyrrolo[2,3-c]pyridin-6-yl)-N-(3-phenylpropyl)-1H-benzo[d]imidazole-1-carboxamide

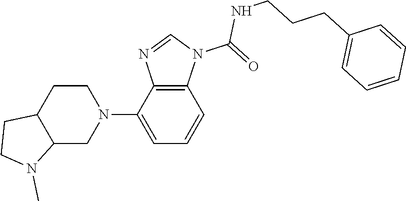 [SMILES: CN1CCC2CCN(c3cccc4c3ncn4C(=O)NCCCc3ccccc3)CC21]

Following general procedure E, 3-fluoro-2-nitroaniline (279 mg, 1.8 mmol) and 1-methyloctahydro-1H-pyrrolo[2,3-c]pyridine (250 mg, 1.8 mmol) afforded 3-(1-methyloctahydro-6H-pyrrolo[2,3-c]pyridin-6-yl)-2-nitroaniline (333 mg, 67%) as a yellow oil. LC-MS m/z: 277.2 [M+H]⁺; t_{R}=1.59 min.

Following general procedure F, 3-(1-methyloctahydro-6H-pyrrolo[2,3-c]pyridin-6-yl)-2-nitroaniline (333 mg, 1.2 mmol) afforded 3-(1-methyloctahydro-6H-pyrrolo[2,3-c]pyridin-6-yl)benzene-1,2-diamine (290 mg, 98%) as a yellow oil. LC-MS m/z: 247.2 [M+H]⁺; t_{R}=1.38 min.

Following general procedure G (method B), 3-(1-methyloctahydro-6H-pyrrolo[2,3-c]pyridin-6-yl)benzene-1,2-diamine (290 mg, 1.2 mmol) afforded 4-(1-methyloctahydro-6H-pyrrolo[2,3-c]pyridin-6-yl)-1H-benzo[d]imidazole (250 mg, 83%) as a yellow oil. LC-MS m/z: 257.2 [M+H]⁺. Purity (214 nm): 84%; t_{R}=1.49 min.

Following general procedure C, 4-(1-methyloctahydro-6H-pyrrolo[2,3-c]pyridin-6-yl)-1H-benzo[d]imidazole (90 mg, 0.35 mmol) and 3-phenylpropan-1-amine (47 mg, 0.35 mmol) afforded the title compound (4.9 mg, 3.3%) as a white solid. ¹H NMR (400 MHz, CDCl₃) δ 8.25 (s, 1H), 7.34-7.28 (m, 2H), 7.26-7.18 (m, 4H), 7.11 (d, J=8.1 Hz, 1H), 6.37 (d, J=7.9 Hz, 2H), 5.22-5.19 (m, 1H), 3.77 (s, 1H), 3.70 (t, J=7.6 Hz, 1H), 3.60-3.47 (m, 3H), 3.10 (d, J=10.8 Hz, 1H), 2.76 (t, J=7.5 Hz, 2H), 2.71-2.61 (m, 1H), 2.63 (s, 3H), 2.53 (bs, 1H), 2.36 (s, 2H), 2.14-1.98 (m, 4H), 1.92 (d, J=14.6 Hz, 1H). LC-MS m/z: 418.2 [M+H]⁺. Purity (214 nm): 100%; t_{R}=9.57 min.

### Example 272—4-(3-((Dimethylamino)methyl)azetidin-1-yl)-N-(3-phenylpropyl)-1H-benzo[d]imidazole-1-carboxamide

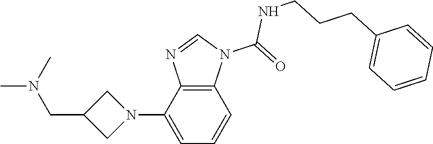 [SMILES: CN(C)CC1CN(c2cccc3c2ncn3C(=O)NCCCc2ccccc2)C1]

Following general procedure E, 3-fluoro-2-nitroaniline (821 mg, 5.26 mmol) and 1-(azetidin-3-yl)-N,N-dimethylmethanamine (600 mg, 5.26 mmol) afforded 3-(3-((dimethylamino)methyl)azetidin-1-yl)-2-nitroaniline (500 mg, 38%) as an orange solid. LC-MS m/z: 251.2 [M+H]⁺. Purity (214 nm): 96%; t_{R}=1.65 min.

Following general procedure F, 3-(3-((dimethylamino)methyl)azetidin-1-yl)-2-nitroaniline (250 mg, 1.0 mmol) afforded 3-(4-methyl-1,4-diazepan-1-yl)benzene-1,2-diamine (220 mg, 100%) as a pale oil. LC-MS m/z: 221.2 [M+H]⁺; t_{R}=1.28 min.

Following general procedure G (method B), 3-(4-methyl-1,4-diazepan-1-yl)benzene-1,2-diamine (220 mg, 1.0 mmol) afforded 1-(1-(1H-benzo[d]imidazol-4-yl)azetidin-3-yl)-N,N-dimethyl methanamine (50 mg, 24%) as a colorless oil. LC-MS m/z: 231.2 [M+H]⁺. Purity (214 nm): 95%; t_{R}=1.40 min.

Following general procedure C, 1-(1-(1H-benzo[d]imidazol-4-yl)azetidin-3-yl)-N,N-dimethyl methanamine (50 mg, 0.22 mmol) and 3-phenylpropan-1-amine (30 mg, 0.22 mmol) afforded the title compound (62.0 mg, 73.0%) as a yellow solid. ¹H NMR (400 MHz, CDCl₃) δ 8.31 (s, 1H), 7.32-7.26 (m, 2H), 7.24-7.11 (m, 5H), 7.07 (bs, 1H), 6.17 (d, J=6.9 Hz, 1H), 4.35 (bs, 2H), 3.88 (bs, 2H), 3.49 (d, J=5.6 Hz, 2H), 3.06 (s, 3H), 2.73 (t, J=7.4 Hz, 2H), 2.55 (bs, 6H), 2.03 (p, J=7.8 Hz, 2H). LC-MS m/z: 392.3 [M+H]⁺. Purity (214 nm): 100%; t_{R}=9.19 min.

### Example 273—N-(3-Phenylpropyl)-4-(3-(trifluoromethyl)azetidin-1-yl)-1H-benzo[d]imidazole-1-carboxamide

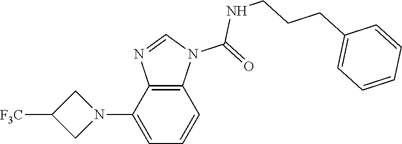 [SMILES: O=C(NCCCc1ccccc1)n1cnc2c(N3CC(C(F)(F)F)C3)cccc21]

A suspension of 3-fluoro-2-nitroaniline (106 mg, 0.68 mmol), 3-(trifluoromethyl)azetidine (100 mg, 0.62 mmol) and K₂CO₃ (429 mg, 3.10 mmol) in CH₃CN (10 mL) was stirred at 80° C. for 15 h. The reaction mixture was quenched with water (30 mL), extracted with DCM/MeOH (10/1; 2×30 mL), concentrated and purified by silica gel column chromatography (DCM:MeOH=1:0) to give 2-nitro-3-(3-(trifluoromethyl)azetidin-1-yl)aniline (150 mg, 92%) as an orange solid. LC-MS m/z: 262.1 [M+H]⁺. Purity (214 nm): 95%; t_{R}=1.72 min.

Following general procedure F, 2-nitro-3-(3-(trifluoromethyl)azetidin-1-yl)aniline (150 mg, 0.57 mmol) afforded 3-(3-(trifluoromethyl)azetidin-1-yl)benzene-1,2-diamine (100 mg, 76%) as a colorless oil. LC-MS m/z: 232.1 [M+H]⁺; t_{R}=1.53 min.

Following general procedure G (method B), 3-(3-(trifluoromethyl)azetidin-1-yl)benzene-1,2-diamine (100 mg, 0.43 mmol) afforded 4-(3-(trifluoromethyl)azetidin-1-yl)-1H-benzo[d]imidazole (68 mg, 66%) as a white solid. LC-MS m/z: 242.1 [M+H]⁺. Purity (214 nm): 90%; t_{R}=1.79 min.

Following general procedure C, 4-(3-(trifluoromethyl)azetidin-1-yl)-1H-benzo[d]imidazole (68 mg, 0.28 mmol) and 3-phenylpropan-1-amine (38 mg, 0.28 mmol) afforded the title compound (65.7 mg, 58.3%) as a white solid. ¹H NMR (400 MHz, CDCl₃) δ 8.17 (s, 1H), 7.32 (dd, J=9.7, 5.1 Hz, 2H), 7.27-7.18 (m, 3H), 7.02 (d, J=8.1 Hz, 1H), 6.28 (d, J=7.9 Hz, 1H), 5.69 (bs, 1H), 4.46 (t, J=8.5 Hz, 2H), 4.33 (dd, J=8.4, 6.1 Hz, 2H), 3.57 (dd, J=12.9, 6.9 Hz, 2H), 3.48-3.42 (m, 1H), 2.80 (t, J=7.4 Hz, 2H), 2.08 (p, J=7.2 Hz, 2H). LC-MS m/z: 403.0 [M+H]⁺. Purity (214 nm): 99.46%; t_{R}=10.99 min.

### Example 274—4-(2-Fluoroaziridin-1-yl)-N-(3-phenylpropyl)-1H-benzo[d]imidazole-1-carboxamide

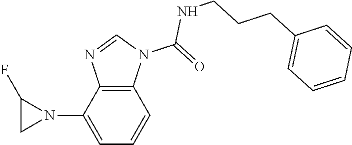 [SMILES: O=C(NCCCc1ccccc1)n1cnc2c(N3CC3F)cccc21]

Following general procedure C, 4-(2-fluoroaziridin-1-yl)-1H-benzo[d]imidazole (25 mg, 0.14 mmol) and 3-phenylpropan-1-amine (28 mg, 0.21 mmol) afforded the title compound (3.1 mg, 7%) as a white solid. ¹H NMR (400 MHz, CDCl₃) δ 8.18 (s, 1H), 7.39 (d, J=7.7 Hz, 1H), 7.30 (dd, J=12.0, 4.8 Hz, 3H), 7.25-7.19 (m, 3H), 6.91 (d, J=7.8 Hz, 1H), 5.65 (t, J=4.9 Hz, 1H), 5.30 (ddd, J=75.8, 3.9, 1.5 Hz, 1H), 3.56 (dd, J=12.8, 6.8 Hz, 2H), 2.89 (s, 1H), 2.78 (t, J=7.3 Hz, 2H), 2.34 (d, J=3.9 Hz, 1H), 2.07 (p, J=7.2 Hz, 2H). LC-MS m/z: 339.0 [M+H]⁺. HPLC Purity (214 nm): 96%; t_{R}=8.58 min.

### Example 275—4-(6-Oxohexahydropyrrolo[1,2-a]pyrazin-2(1H)-yl)-N-(3-phenylpropyl)-1H-benzo[d]imidazole-1-carboxamide

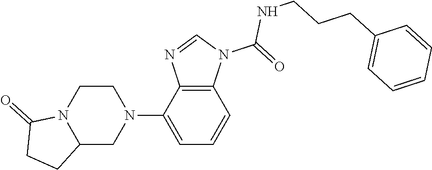 [SMILES: O=C1CCC2CN(c3cccc4c3ncn4C(=O)NCCCc3ccccc3)CCN12]

Following general procedure E, 3-fluoro-2-nitroaniline (500 mg, 3.20 mmol) and hexahydropyrrolo[1,2-a]pyrazin-6(2H)-one (539 mg, 3.84 mmol) afforded 2-(3-amino-2-nitrophenyl)hexahydropyrrolo[1,2-a]pyrazin-6(2H)-one (410 mg, 46.3%) as a light brown semisolid. LC-MS m/z: 277.2 [M+H]⁺. Purity (214 nm): 100%; t_{R}=1.38 min.

Following general procedure F, 2-(3-amino-2-nitrophenyl)hexahydropyrrolo[1,2-a]pyrazin-6(2H)-one (410 mg, 1.48 mmol) afforded crude 2-(2,3-diaminophenyl)hexa hydropyrrolo[1,2-a]pyrazin-6(2H)-one (370 mg) as a gray solid. LC-MS m/z: 247.2 [M+H]⁺. Purity (254 nm): 94.78%; t_{R}=1.27 min.

Following general procedure G (method A), 2-(2,3-diaminophenyl)hexahydropyrrolo[1,2-a]pyrazin-6(2H)-one (370 mg, 1.50 mmol) afforded crude 2-(1H-benzo[d]imidazol-4-yl)hexahydropyrrolo[1,2-a]pyrazin-6(2H)-one (390 mg) as a light brown solid. LC-MS m/z: 257.2 [M+H]⁺. Purity (214 nm): 100%; t_{R}=0.80 min.

Following general procedure C, 2-(1H-benzo[d]imidazol-4-yl)hexahydropyrrolo[1,2-a]pyrazin-6(2H)-one (170 mg, 0.66 mmol) and 3-phenylpropan-1-amine (90 mg, 0.66 mmol) afforded the title compound (49.1 mg, 17.7%) as a white solid. ¹H NMR (400 MHz, CDCl₃) δ 8.21 (s, 1H), 7.30 (dd, J=12.5, 4.4 Hz, 4H), 7.25-7.14 (m, 3H), 6.74 (dd, J=6.9, 1.9 Hz, 1H), 5.75 (bs, 1H), 4.35 (dd, J=11.5, 2.0 Hz, 1H), 4.22-4.04 (m, 2H), 4.03-3.90 (m, 1H), 3.57 (dd, J=12.8, 6.9 Hz, 2H), 3.21 (dt, J=10.7, 2.8 Hz, 1H), 2.76-2.68 (m, 3H), 2.59-2.40 (m, 3H), 2.37-2.22 (m, 1H), 2.07 (p, J=7.3 Hz, 2H), 1.75-1.64 (m, 1H). LC-MS m/z: 418.2 [M+H]⁺. HPLC Purity (214 nm): 100%; t_{R}=8.30 min.

### Example 276—4-(3-(4-Methylpiperazin-1-yl)azetidin-1-yl)-N-(3-phenylpropyl)-1H-benzo[d]imidazole-1-carboxamide

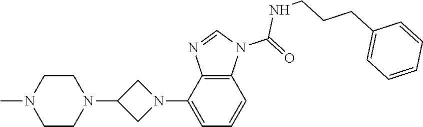 [SMILES: CN1CCN(C2CN(c3cccc4c3ncn4C(=O)NCCCc3ccccc3)C2)CC1]

Following general procedure C, 4-(3-(4-methylpiperazin-1-yl)azetidin-1-yl)-1H-benzo[d]imidazole (200 mg, 0.74 mmol) and 3-phenylpropan-1-amine (100 mg, 0.74 mmol) afforded the title compound (156.4 mg, 49.1%) as a white solid. ¹H NMR (400 MHz, CDCl₃) δ 8.23 (s, 1H), 7.35-7.29 (m, 2H), 7.27-7.21 (m, 3H), 7.19 (d, J=8.0 Hz, 1H), 7.02-6.91 (m, 1H), 6.27 (d, J=7.9 Hz, 1H), 5.80 (bs, 1H), 4.38 (t, J=7.3 Hz, 2H), 4.13-4.01 (m, 2H), 3.57 (dd, J=13.1, 6.6 Hz, 2H), 3.46-3.37 (m, 1H), 2.79 (t, J=7.5 Hz, 3H), 2.63 (bs, 7H), 2.42 (s, 3H), 2.06 (p, J=7.3 Hz, 2H). LC-MS m/z: 433.0 [M+H]⁺. HPLC Purity (254 nm): 100%; t_{R}=6.31 min.

### Example 277—4-(3-(Fluoromethyl)-4-methylpiperazin-1-yl)-N-(3-phenylpropyl)-1H-benzo[d]imidazole-1-carboxamide

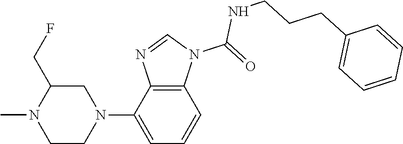 [SMILES: CN1CCN(c2cccc3c2ncn3C(=O)NCCCc2ccccc2)CC1CF]

Following general procedure C, 4-(3-(fluoromethyl)-4-methylpiperazin-1-yl)-1H-benzo[d]imidazole (200 mg, 0.81 mmol) and 3-phenylpropan-1-amine (109 mg, 1 mmol) afforded the title compound (11.1 mg, 3.4%) as a white solid. ¹H NMR (400 MHz, CDCl₃) δ 8.23 (d, J=3.6 Hz, 2H), 7.35-7.28 (m, 3H), 7.25-7.18 (m, 3H), 6.73 (d, J=7.7 Hz, 1H), 5.85 (bs, 1H), 4.87-4.61 (m, 2H), 4.09 (d, J=9.5 Hz, 1H), 3.99 (d, J=9.6 Hz, 1H), 3.58 (q, J=6.8 Hz, 2H), 3.32-3.10 (m, 4H), 2.92 (t, J=10.9 Hz, 1H), 2.78 (t, J=7.4 Hz, 2H), 2.64 (s, 3H), 2.05 (p, J=7.4 Hz, 2H). LC-MS m/z: 410.2 [M+H]⁺. HPLC Purity (214 nm): 100%; t_{R}=6.93 min.

### Example 278—4-(4-(2-Cyanoethyl)piperazin-1-yl)-N-(3-phenylpropyl)-1H-benzo[d]imidazole-1-carboxamide

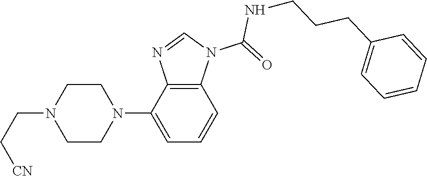 [SMILES: N#CCCN1CCN(c2cccc3c2ncn3C(=O)NCCCc2ccccc2)CC1]

Following general procedure C, 3-(4-(1H-benzo[d]imidazol-4-yl)-piperazin-1-yl)propanenitrile (200 mg, 0.8 mmol) and 3-phenylpropan-1-amine (108 mg, 0.8 mmol) afforded the title compound (76.7 mg, 23.5%) as white solid. ¹H NMR (400 MHz, CDCl₃) δ 8.21 (s, 1H), 7.40-7.13 (m, 7H), 6.73 (dd, J=6.6, 2.2 Hz, 1H), 5.70 (bs, 1H), 3.59-3.52 (m, 6H), 2.91-2.72 (m, 8H), 2.59 (t, J=7.0 Hz, 2H), 2.06 (p, J=7.2 Hz, 2H). LC-MS m/z: 416.7 [M+H]⁺. HPLC Purity (214 nm): 100%; t_{R}=6.32 min.

### Example 279—4-(Azetidin-1-yl)-N-(3-phenylpropyl)-1H-benzo[d]-imidazole-1-carboxamide

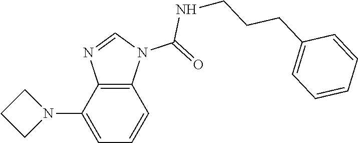 [SMILES: O=C(NCCCc1ccccc1)n1cnc2c(N3CCC3)cccc21]

To a suspension of 3-fluoro-2-nitroaniline (500 mg, 3.20 mmol) and K₂CO₃ (1.1 g, 8.01 mmol) in CH₃CN (10 mL) was added azetidine (274 mg, 4.80 mmol). The reaction mixture was stirred at 90° C. under N₂ for 16 h. The mixture was concentrated, and the residue was purified by silica gel column chromatography (PE:EA=3:1) to give 3-(azetidin-1-yl)-2-nitroaniline (630 mg, 99%) as a brown semisolid. LC-MS m/z: 194.1 [M+H]⁺. Purity (214 nm): 100%; t_{R}=1.26 min.

Following general procedure F, 3-(azetidin-1-yl)-2-nitroaniline (630 mg, 3.261 mmol) afforded 3-(azetidin-1-yl)benzene-1,2-diamine (550 mg, crude) as a gray solid. LC-MS m/z: 164.1 [M+H]⁺. Purity (254 nm): 99.16%; t_{R} 1.27 min.

Following general procedure G (method A), 3-(azetidin-1-yl)benzene-1,2-diamine (500 mg, 3.063 mmol) afforded 4-(azetidin-1-yl)-1H-benzo[d]imidazole (365 mg, crude) as a brown solid. LC-MS m/z: 174.1 [M+H]⁺. Purity (214 nm): 95.97%; t_{R}=1.36 min.

Following general procedure C, 4-(azetidin-1-yl)-1H-benzo[d]-imidazole (200 mg, 1.155 mmol) and 3-phenylpropan-1-amine (156 mg, 3.464 mmol) afforded the title compound (97.3 mg, 25.2%) as a white solid. ¹H NMR (400 MHz, CDCl₃) δ 8.18 (s, 1H), 7.34-7.27 (m, 2H), 7.20 (dd, J=15.7, 7.7 Hz, 4H), 6.90 (d, J=8.0 Hz, 1H), 6.24 (d, J=7.9 Hz, 1H), 5.70 (bs, 1H), 4.25 (t, J=7.3 Hz, 4H), 3.54 (dd, J=13.0, 6.9 Hz, 2H), 2.77 (t, J=7.5 Hz, 2H), 2.50 (p, J=10.4 Hz, 2H), 2.07 (p, J=7.5 Hz, 2H). LC-MS m/z: 335.2 [M+H]⁺. HPLC Purity (214 nm): 100%, t_{R}=8.63 min.

### Example 280—N-(4-Methylpent-2-ynyl)-4-(4-methylpiperazin-1-yl)-1H-benzo[d]imidazole-1-carboxamide

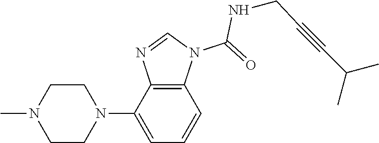 [SMILES: CC(C)C#CCNC(=O)n1cnc2c(N3CCN(C)CC3)cccc21]

Following general procedure C, 4-(4-methylpiperazin-1-yl)-1H-benzo[d]imidazole (200 mg, 0.93 mmol) and 4-methylpent-2-yn-1-amine (90 mg, 0.93 mmol) afforded the title compound (13.6 mg, 4.3%) as a white solid. ¹H NMR (400 MHz, CDCl₃) δ 8.55 (s, 1H), 7.59 (d, J=8.0 Hz, 1H), 7.32 (d, J=8.1 Hz, 1H), 7.03 (bs, 1H), 6.75 (d, J=7.9 Hz, 1H), 4.28 (dd, J=5.0, 2.0 Hz, 2H), 3.74 (bs, 4H), 3.22 (bs, 4H), 2.71 (s, 3H), 2.65-2.57 (m, 1H), 1.19 (d, J=6.9 Hz, 6H). LC-MS m/z: 340.0 [M+H]⁺. HPLC Purity (254 nm): 100%; t_{R}=5.95 min.

### Example 281—N-(But-2-yn-1-yl)-4-(4-methylpiperazin-1-yl)-1H-benzo[d]imidazole-1-carboxamide

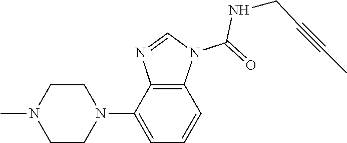 [SMILES: CC#CCNC(=O)n1cnc2c(N3CCN(C)CC3)cccc21]

Following general procedure C, 4-(4-methylpiperazin-1-yl)-1H-benzo[d]-imidazole (103 mg, 0.5 mmol) and but-2-yn-1-amine (300 mg, 4.2 mmol) afforded the title compound (44.2 mg, 12.4%) as a white solid. ¹H NMR (400 MHz, DMSO) δ 9.03 (bs, 1H), 8.16 (s, 1H), 7.61 (d, J=8.1 Hz, 1H), 7.21 (t, J=8.1 Hz, 1H), 6.69 (d, J=7.9 Hz, 1H), 4.08 (d, J=2.4 Hz, 2H), 3.52 (bs, 4H), 2.68 (bs, 4H), 2.35 (s, 3H), 1.81 (s, 3H). LC-MS m/z: 311.7 [M+H]⁺. HPLC Purity (214 nm): 100%; t_{R}=5.19 min.

### Example 282—4(4-Methylpiperazin-1-yl)-N-(3-phenylprop-2-yn-1-yl)-1H-benzo[d]imidazole-1-carboxamide

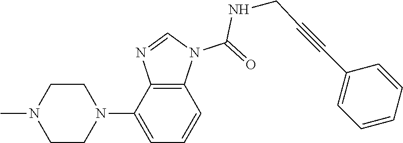 [SMILES: CN1CCN(c2cccc3c2ncn3C(=O)NCC#Cc2ccccc2)CC1]

Following general procedure C, 4-(4-methylpiperazin-1-yl)-1H-benzo[d]imidazole (200 mg, 0.92 mmol) and 3-phenylprop-2-yn-1-amine hydrochloride (155 mg, 0.92 mmol) afforded the title compound (29.1 mg, 8.4%) as a white solid. ¹H NMR (400 MHz, DMSO-d₆) δ 9.17 (t, J=5.2 Hz, 1H), 8.62 (s, 1H), 7.62 (d, J=8.1 Hz, 1H), 7.46 (dd, J=6.8, 3.0 Hz, 2H), 7.43-7.35 (m, 3H), 7.22 (t, J=8.1 Hz, 1H), 6.68 (d, J=7.9 Hz, 1H), 4.40 (d, J=5.3 Hz, 2H), 3.48 (bs, 4H), 2.53 (bs, 4H), 2.25 (s, 3H). LC-MS m/z: 374.1 [M+H]⁺. HPLC Purity (214 nm): 100%; t_{R}=6.17 min.

### Example 283—N-(3-Cyclopropylprop-2-ynyl)-4-(4-methylpiperazin-1-yl)-1H-benzo[d]imidazole-1-carboxamide

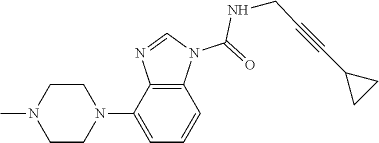 [SMILES: CN1CCN(c2cccc3c2ncn3C(=O)NCC#CC2CC2)CC1]

Following general procedure C, 4-(4-methylpiperazin-1-yl)-1H-benzo[d]imidazole (216 mg, 1.0 mmol) and 3-cyclopropylprop-2-yn-1-amine (100 mg, 1.1 mmol) afforded the title compound (10.8 mg, 3.2%) as a white solid. ¹H NMR (400 MHz, CDCl₃) δ 9.05 (t, J=5.6 Hz, 1H), 8.63 (s, 1H), 8.15 (s, 1H), 7.62 (d, J=8.0 Hz, 1H), 7.21 (t, J=8.4 Hz, 1H), 6.69 (d, J=8.0 Hz, 1H), 4.07 (d, J=4.0 Hz, 1H), 3.50 (bs, 4H), 2.75 (bs, 4H), 2.40 (s, 3H), 1.37-1.30 (m, 1H), 0.78-0.73 (m, 2H), 0.62-0.58 (m, 2H). LC-MS m/z: 338.3 [M+H]⁺. HPLC Purity (214 nm): 97.20%; t_{R}=7.89 min.

### Example 284—N-(3-Cyclopropylprop-2-ynyl)-4-(4-methylpiperazin-1-yl)-1H-benzo[d]imidazole-1-carboxamide

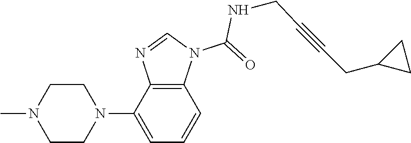 [SMILES: CN1CCN(c2cccc3c2ncn3C(=O)NCC#CCC2CC2)CC1]

Following general procedure C, 4-(4-methylpiperazin-1-yl)-1H-benzo[d]imidazole (216 mg, 1.0 mmol) and 4-cyclopropylbut-2-yn-1-amine hydrochloride (110 mg, 1.0 mmol) afforded the title compound (5.8 mg, 1.6%) as a yellow solid. ¹H NMR (400 MHz, CDCl₃) δ 8.14 (s, 1H), 7.17 (d, J=8.4 Hz, 1H), 7.05 (t, J=8.0 Hz, 1H), 6.53 (d, J=8.0 Hz, 1H), 5.92 (t, J=4.8 Hz, 1H), 4.07-4.04 (m, 2H), 3.36 (bs, 4H), 2.55-2.53 (m, 4H), 2.23 (s, 3H), 2.03-2.01 (m, 2H), 0.71-0.69 (m, 1H), 0.29-0.24 (m, 2H), 0.11-0.01 (m, 2H). LC-MS m/z: 351.7 [M+H]⁺. HPLC Purity (214 nm): 98.39%; t_{R}=5.62 min.

### Example 285—4-(4-Methylpiperazin-1-yl)-N-(4-(1-(trifluoromethyl)cyclopropyl)but-2-yn-1-yl)-1H-benzo[d]imidazole-1-carboxamide

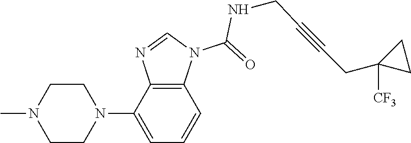 [SMILES: CN1CCN(c2cccc3c2ncn3C(=O)NCC#CCC2(C(F)(F)F)CC2)CC1]

Following general procedure C, 4-(4-methylpiperazin-1-yl)-1H-benzo[d]imidazole (54 mg, 0.25 mmol) and 4-(1-(trifluoromethyl)cyclopropyl)but-2-yn-1-amine (44 mg, 0.25 mmol) afforded the title compound (6.5 mg, 6.2%/o) as a white solid. ¹H NMR (400 MHz, CDCl₃) δ 8.48 (s, 1H), 7.52 (d, J=8.1 Hz, 1H), 7.30 (t, J=7.3 Hz, 1H), 6.96 (bs, 1H), 6.74 (d, J=7.9 Hz, 1H), 4.26 (dd, J=4.8, 2.4 Hz, 2H), 3.69 (bs, 4H), 3.15 (bs, 4H), 2.71 (s, 2H), 2.65 (s, 3H), 1.01-0.92 (m, 2H), 0.84 (s, 2H). LC-MS m/z: 420.0 [M+H]⁺. HPLC Purity (214 nm): 100%; t_{R}=6.29 min.

### Example 286—4-(4-(Oxetan-3-yl)piperazin-1-yl)-N-(3-phenylpropyl)-1H-benzo[d]imidazole-1-carboxamide

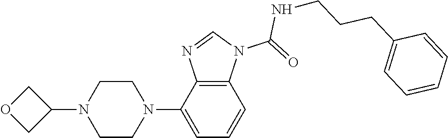 [SMILES: O=C(NCCCc1ccccc1)n1cnc2c(N3CCN(C4COC4)CC3)cccc21]

Following general procedure C, 4-(4-(oxetan-3-yl)piperazin-1-yl)-1H-benzo[d]imidazole (100 mg, 0.39 mmol) and 3-phenylpropan-1-amine (53 mg, 0.39 mmol) afforded the title compound (13.6 mg, 8.4%) as a white solid. ¹H NMR (400 MHz, CDCl₃) δ 8.22 (s, 1H), 7.32 (dd, J=13.3, 5.9 Hz, 4H), 7.27-7.14 (m, 3H), 6.77 (d, J=7.3 Hz, 1H), 5.73 (bs, 1H), 4.74 (d, J=6.7 Hz, 4H), 3.80-3.47 (m, 7H), 2.80 (t, J=7.3 Hz, 2H), 2.68 (bs, 4H), 2.07 (dd, J=14.4, 7.3 Hz, 2H). LC-MS m/z: 420.1 [M+H]⁺. HPLC Purity (254 nm): 100%; t_{R}=6.22 min.

### Example 287—N-(2-(3,3-Difluorocyclopentyl)ethyl)-4-(4-(1-methylazetidin-3-yl)piperazin-1-yl)-1H-benzo[d]imidazole-1-carboxamide

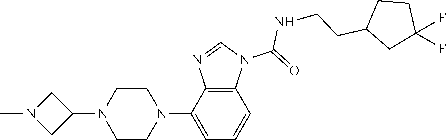 [SMILES: CN1CC(N2CCN(c3cccc4c3ncn4C(=O)NCCC3CCC(F)(F)C3)CC2)C1]

Following general procedure C, 4-(4-(1-methylazetidin-3-yl)piperazin-1-yl)-1H-benzo[d]imidazole (80 mg, 0.3 mmol) and 2-(3,3-difluorocyclopentyl)ethan-1-amine (45 mg, 0.3 mmol) afforded the title compound (26.0 mg, 19.7%) as a white solid. ¹H NMR (400 MHz, CDCl₃) δ 7.82 (s, 1H), 7.61 (d, J=8.1 Hz, 1H), 7.29 (s, 1H), 6.68 (d, J=7.4 Hz, 1H), 4.19 (bs, 2H), 3.42 (m, 7H), 3.36 (bs, 2H), 2.74 (s, 3H), 2.55 (bs, 4H), 2.42-2.26 (m, 1H), 2.24-2.15 (m, 2H), 2.03 (bs, 2H), 1.82-1.74 (m, 3H), 1.58-1.36 (m, 1H). LC-MS m/z: 447.1 [M+H]⁺. HPLC Purity (214 nm): 100%; t_{R}=5.93 min.

### Example 288—4-(4-(2-Cyanoethyl)piperazin-1-yl)-N-(2-(3,3-difluorocyclopentyl)ethyl)-1H-benzo[d]imidazole-1-carboxamide

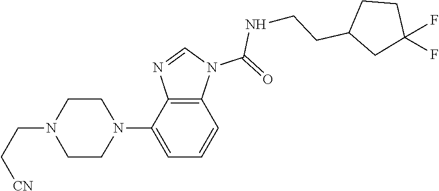 [SMILES: N#CCCN1CCN(c2cccc3c2ncn3C(=O)NCCC2CCC(F)(F)C2)CC1]

Following general procedure C, 4-(4-(2-isocyanoethyl)piperazin-1-yl)-1H-benzo[d]imidazole (110 mg, 0.431 mmol) and 2-(3,3-difluorocyclopentyl)ethan-1-amine hydrochloride (80 mg, 0.431 mmol) afforded the title compound (72.2 mg, 38.9%) as a pink solid. ¹H NMR (400 MHz, CDCl₃) δ 8.37 (s, 1H), 7.40-7.27 (m, 2H), 6.75 (d, J=7.6 Hz, 1H), 5.95 (t, J=5.4 Hz, 1H), 3.64-3.41 (m, 6H), 2.82 (m, 6H), 2.60 (t, J=7.0 Hz, 2H), 2.35 (m, 1H), 2.28-2.12 (m, 2H), 2.12-1.97 (m, 2H), 1.80 (q, J=7.6 Hz, 2H), 1.78-1.71 (m, 1H), 1.59-1.39 (m, 1H). LC-MS m/z: 431.1 [M+H]⁺. HPLC Purity (214 nm): 100%; t_{R}=6.16 min.

### Example 289—N-(2-(3,3-Difluorocyclopentyl)ethyl)-4-(8-methyl-3,8-diazabicyclo[3.2.1]octan-3-yl)-1H-benzo[d]imidazole-1-carboxamide

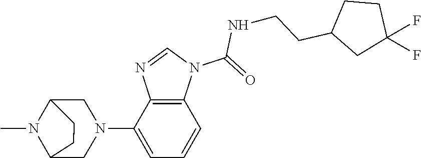 [SMILES: CN1C2CCC1CN(c1cccc3c1ncn3C(=O)NCCC1CCC(F)(F)C1)C2]

Following general procedure C, 4-(8-methyl-3,8-diazabicyclo[3.2.1]octan-3-yl)-1H-benzo[d]imidazole (60 mg, 0.25 mmol) and 2-(3,3-difluorocyclopentyl)ethan-1-amine hydrogen chloride (55 mg, 0.30 mmol) afforded the title compound (24 mg, 23%) as a white solid. ¹H NMR (400 MHz, CDCl₃) δ 8.67 (s, 1H), 7.89 (bs, 1H), 7.62 (d, J=8.0 Hz, 1H), 7.22 (d, J=8.0 Hz, 1H), 6.61 (d, J=8.0 Hz, 1H), 4.22 (d, J=12.0 Hz, 2H), 3.98 (s, 1H), 3.1 (d, J=12.0 Hz, 2H), 3.45 (q, J=6.8 Hz, 2H), 2.78 (s, 3H), 2.43 (d, J=8.0 Hz, 2H), 2.37-2.22 (m, 6H), 2.19-1.98 (m, 2H). 1.79-1.70 (m, 2H), 1.49-1.42 (m, 1H). LC-MS m/z: 418.0 [M+H]⁺. HPLC Purity (214 nm): 100%; t_{R}=6.96 min.

### Example 290—N-(2-(3,3-Difluorocyclopentyl)ethyl)-4-(hexahydropyrrolo[1,2-a]pyrazin-2(1H)-yl)-1H-benzo[d]imidazole-1-carboxamide

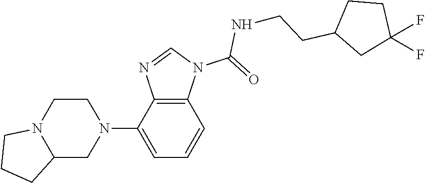 [SMILES: O=C(NCCC1CCC(F)(F)C1)n1cnc2c(N3CCN4CCCC4C3)cccc21]

Following general procedure C, 4-(hexahydropyrrolo[1,2-a]pyrazin-2(1H)-yl)-1H-benzo[d]imidazole (70 mg, 0.3 mmol) and 2-(3,3-difluorocyclopentyl)ethan-1-amine (59.0 mg, 0.4 mmol) afforded the title compound (53.7 mg, 43%) as a white solid. ¹H NMR (400 MHz, CDCl₃) δ 8.60 (s, 1H), 7.87 (bs, 1H), 7.63 (d, J=8.2 Hz, 1H), 7.24 (d, J=8.1 Hz, 1H), 6.69 (d, J=8.0 Hz, 11H), 4.04 (bs, 2H), 3.51-3.34 (m, 7H), 3.16 (bs, 1H), 2.91 (bs, 1H), 2.41-2.28 (m, 1H), 2.27-2.09 (m, 8H), 1.84-1.65 (m, 3H), 1.51-1.46 (m, 1H). LC-MS m/z: 418.2 [M+H]⁺. HPLC Purity (214 nm): 100%, t_{R}=6.21 min.

### Example 291—N-(2-(3,3-Difluorocyclopentyl)ethyl)-4-(4-ethylpiperazin-1-yl)-1H-benzo[d]imidazole-1-carboxamide

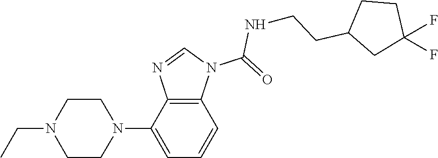 [SMILES: CCN1CCN(c2cccc3c2ncn3C(=O)NCCC2CCC(F)(F)C2)CC1]

Following general procedure C, 4-(4-ethylpiperazin-1-yl)-1H-benzo[d]imidazole (230 mg, 1.0 mmol) and 2-(3,3-difluorocyclopentyl)ethan-1-amine (80 mg, 0.5 mmol) afforded the title compound (31.3 mg, 13.7%) as a white solid. ¹H NMR (400 MHz, CDCl₃) δ 8.72 (s, 1H), 8.22 (bs, 1H), 7.72 (d, J=8.2 Hz, 1H), 7.24 (d, J=8.1 Hz, 1H), 6.70 (d, J=7.9 Hz, 1H), 3.75 (bs, 4H), 3.45 (dd, J=13.6, 6.2 Hz, 2H), 3.34 (bs, 4H), 3.03 (d, J=7.1 Hz, 2H), 2.38-2.25 (m, 1H), 2.25-2.10 (m, 2H), 2.06-1.99 (m, 2H), 1.79 (q, J=6.8 Hz, 2H), 1.76-1.65 (m, 1H), 1.54-1.43 (m, 1H), 1.42-1.37 (m, 3H). LC-MS m/z: 405.7 [M+H]⁺. HPLC Purity (214 nm): 100%; t_{R}=6.12 min.

### Example 292—N-(2-(3,3-Difluorocyclopentyl)ethyl)-4-(5-methyl-2,5-diazabicyclo[2.2.2]octan-2-yl)-1H-benzo[d]imidazole-1-carboxamide

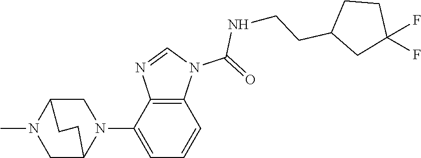 [SMILES: CN1CC2CCC1CN2c1cccc2c1ncn2C(=O)NCCC1CCC(F)(F)C1]

Following general procedure C, 2-(1H-benzo[d]imidazol-4-yl)-5-methyl-2,5-diazabicyclo[2.2.2]octane (50 mg, 0.21 mmol) and 2-(3,3-difluorocyclopentyl)ethan-1-amine hydrogen chloride (47 mg, 0.25 mmol) afforded the title compound (39.4 mg, 45.0%) as a yellow solid. ¹H NMR (400 MHz, CDCl₃) δ 8.57 (s, 1H), 8.17 (s, 1H), 7.52 (d, J=8.1 Hz, 1H), 7.28 (d, J=8.1 Hz, 1H), 6.46 (d, J=8.0 Hz, 1H), 5.48 (s, 1H), 4.17 (d, J=11.0 Hz, 1H), 3.80-3.56 (m, 3H), 3.53-3.42 (m, 2H), 3.33 (d, J=9.4 Hz, 1H), 2.85 (s, 3H), 2.50-2.31 (m, 2H), 2.28-2.14 (m, 3H), 2.12-1.88 (m, 4H), 1.87-1.69 (m, 3H), 1.56-1.42 (m, 1H). LC-MS m/z: 418.0 [M+H]⁺. HPLC Purity (214 nm): 100%; t_{R}=6.33 min.

### Example 293—N-(2-(3,3-difluorocyclopentyl)ethyl)-4-(5-methyl-2,5-diazabicyclo[2.2.1]heptan-2-yl)-1H-benzo[d]imidazole-1-carboxamide

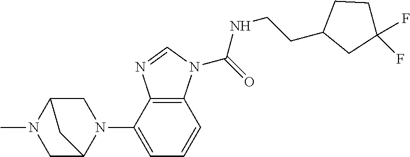 [SMILES: CN1CC2CC1CN2c1cccc2c1ncn2C(=O)NCCC1CCC(F)(F)C1]

Following general procedure C, 4-(5-methyl-2,5-diazabicyclo[2.2.1]heptan-2-yl)-1H-benzo[d]imidazole 50 mg, 0.22 mmol) and 2-(3,3-difluorocyclopentyl)ethanamine (40 mg, 0.22 mmol) afforded the title compound (2.9 mg, 3.2%) as a yellow solid. ¹H NMR (400 MHz, CDCl₃) δ 8.28 (s, 1H), 7.22 (t, J=8.0 Hz, 1H), 6.98 (d, J=8.2 Hz, 1H), 6.38 (d, J=8.0 Hz, 1H), 5.96 (bs, 1H), 5.41 (s, 1H), 3.71 (dd, J=18.8, 9.7 Hz, 3H), 3.51 (dd, J=14.3, 6.5 Hz, 2H), 3.11 (d, J=9.7 Hz, 1H), 2.82 (d, J=10.3 Hz, 1H), 2.48 (s, 3H), 2.39-2.33 (m, 1H), 2.23-2.18 (m, 2H), 2.12-1.97 (m, 4H), 1.84-1.76 (m, 3H), 1.54-1.46 (m, 1H). LC-MS m/z: 404.0 [M+H]⁺. HPLC Purity (214 nm): 100%; t_{R}=6.16 min.

### Example 294—4-(5-Methyl-2,5-diazabicyclo[2.2.2]octan-2-yl)-N-(2-phenoxyethyl)-1H-benzo[d]imidazole-1-carboxamide

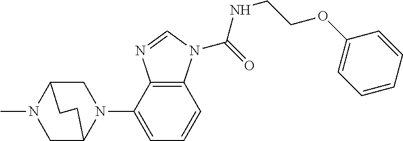 [SMILES: CN1CC2CCC1CN2c1cccc2c1ncn2C(=O)NCCOc1ccccc1]

Following general procedure C, 2-(1H-benzo[d]imidazol-4-yl)-5-methyl-2,5-diazabicyclo[2.2.2]octane (185 mg, 0.76 mmol) and 2-phenoxyethan-1-amine (210 mg, 1.52 mmol) afforded the title compound (10.0 mg, 3.2%) as a yellow solid. ¹H NMR (400 MHz, CDCl₃) δ 8.42 (s, 1H), 7.35-7.25 (m 4H), 7.24 (bs, 1H), 7.01 (t, J=7.6 Hz, 1H), 6.94 (d, J=8.0 Hz, 2H), 6.46 (d, J=7.6 Hz, 1H), 5.50 (bs, 1H), 4.25 (t, J=4.7 Hz, 2H), 4.18-4.08 (m, 1H), 3.93 (d, J=4.3 Hz, 2H), 3.71-3.49 (m, 3H), 3.29 (bs, 1H), 2.82 (s, 3H), 2.41 (bs, 1H), 2.28-2.15 (m, 1H), 2.04-1.83 (m, 2H). LC-MS m/z: 406.0 [M+H]⁺. HPLC Purity (214 nm): 100%; t_{R}=6.18 min.

### Example 295—4-(5-Methyl-2,5-diazabicyclo[2.2.1]heptan-2-yl)-N-(2-phenoxyethyl)-1H-benzo[d]imidazole-1-carboxamide

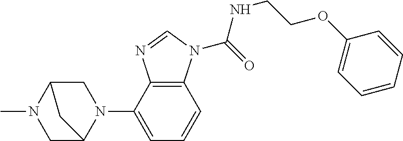 [SMILES: CN1CC2CC1CN2c1cccc2c1ncn2C(=O)NCCOc1ccccc1]

Following general procedure C, 4-(5-methyl-2,5-diazabicyclo[2.2.1]heptan-2-yl)-1H-benzo[d]imidazole (200 mg, 0.88 mmol) and 2-phenoxyethanamine (180 mg, 1.31 mmol) afforded the title compound (45.5 mg, 13.4%) as a white solid. 1H NMR (400 MHz, CDCl₃) δ 8.54 (s, 1H), 8.37 (s, 1H), 7.33-7.28 (m, 3H), 7.20 (t, J=8.0 Hz, 1H), 7.03-6.95 (m, 3H), 6.35 (d, J=7.7 Hz, 1H), 5.58 (bs, 1H), 4.21 (t, J=5.1 Hz, 2H), 4.12 (s, 1H), 3.93-3.86 (m, 2H), 3.86 (dd, J=32.2, 12.8 Hz, 2H), 3.40 (d, J=8.7 Hz, 1H), 3.14 (s, 1H), 2.67 (s, 3H), 2.21 (dd, J=35.9, 10.6 Hz, 2H). LC-MS m/z: 392.3 [M+H]⁺. HPLC Purity (214 nm): 96.32%; t_{R}=6.02 min.

### Example 296—4-(4-(1-Methylazetidin-3-yl)piperazin-1-yl)-N-(2-phenoxyethyl)-1H-benzo[d]imidazole-1-carboxamide

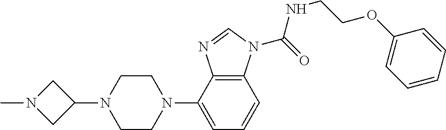 [SMILES: CN1CC(N2CCN(c3cccc4c3ncn4C(=O)NCCOc3ccccc3)CC2)C1]

Following general procedure C, 4-(4-(1-methylazetidin-3-yl)piperazin-1-yl)-1H-benzo[d]imidazole (271 mg, 1 mmol) and 2-phenoxyethan-1-amine (137 mg, 1 mmol) afforded the title compound (77.4 mg, 17.8%) as a white solid. ¹H NMR (400 MHz, CDCl₃) δ 8.58 (s, 1H), 7.49 (d, J=8.1 Hz, 1H), 7.38-7.28 (m, 3H), 7.25 (s, 1H), 6.98 (dd, J=13.4, 6.0 Hz, 1H), 6.93 (d, J=7.8 Hz, 2H), 6.70 (d, J=7.9 Hz, 1H), 4.23 (t, J=5.1 Hz, 2H), 4.17 (t, J=8.0 Hz, 2H), 3.91 (dd, J=10.4, 5.2 Hz, 2H), 3.54 (bs, 4H), 3.46 (t, J=8.4 Hz, 2H), 3.33 (p, J=6.8 Hz, 1H), 2.70 (s, 3H), 2.59-2.55 (m, 4H). LC-MS m/z: 435.2 [M+H]⁺. HPLC Purity (214 nm): 100%; t_{R}=6.48 min.

### Example 297—4-(4-(2-Cyanoethyl)piperazin-1-yl)-N-(2-phenoxy-ethyl)-1H-benzo[d]imidazole-1-carboxamide

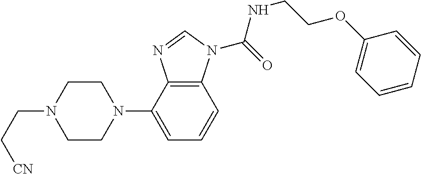 [SMILES: N#CCCN1CCN(c2cccc3c2ncn3C(=O)NCCOc2ccccc2)CC1]

Following general procedure C, 4-(4-(2-isocyanoethyl)piperazin-1-yl)-1H-benzo[d]imidazole (140 mg, 0.548 mmol) and 2-phenoxyethan-1-amine (75 mg, 0.548 mmol) afforded the title compound (107.2 mg, 46.7%) as a pink solid. ¹H NMR (400 MHz, CDCl₃) δ 8.41 (s, 1H), 7.40 (d, J=7.4 Hz, 1H), 7.36-7.28 (m, 3H), 7.01 (t, J=7.4 Hz, 1H), 6.94 (d, J=8.0 Hz, 2H), 6.75 (d, J=7.8 Hz, 1H), 6.43 (t, J=5.4 Hz, 1H), 4.23 (t, J=5.0 Hz, 2H), 3.93 (dd, J=10.3, 5.3 Hz, 2H), 3.59-3.50 (m, 4H), 2.84 (m, 6H), 2.62 (t, J=7.1 Hz, 2H). LC-MS m/z: 419.1 [M+H]⁺. HPLC Purity (214 nm): 100%; t_{R}=6.01 min.

### Example 298—N-(4-Methylpent-2-ynyl)-4-(3-(4-methyl-piperazin-1-yl)azetidin-1-yl)-1H-benzo[d]imidazole-1-carboxamide

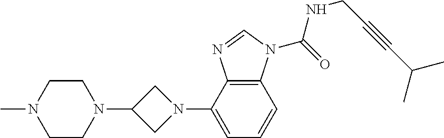 [SMILES: CC(C)C#CCNC(=O)n1cnc2c(N3CC(N4CCN(C)CC4)C3)cccc21]

Following general procedure C, 4-(3-(4-methylpiperazin-1-yl)azetidin-1-yl)-1H-benzo[d]imidazole (100 mg, 0.37 mmol) and 4-methylpent-2-yn-1-amine (72 mg, 0.74 mmol) afforded the title compound (87.9 mg, 60.5%) as a white solid. ¹H NMR (400 MHz, CDCl₃) δ 8.32 (s, 1H), 7.24 (t, J=8.0 Hz, 1H), 7.15 (d, J=8.0 Hz, 1H), 6.32 (bs, 1H), 6.29 (d, J=8.0 Hz, 1H), 4.37 (t, J=7.3 Hz, 2H), 4.28 (dd, J=5.0, 1.9 Hz, 2H), 4.06 (dd, J=7.8, 5.5 Hz, 2H), 3.47-3.39 (m, 1H), 3.27-2.57 (m, 9H), 2.54 (s, 3H), 1.20 (d, J=6.9 Hz, 6H). LC-MS m/z: 395.3 [M+H]⁺. HPLC Purity (254 nm): 100%, t_{R}=6.17 min.

### Example 299—4-(4-(1-Methylazetidin-3-yl)piperazin-1-yl)-N-(4-methylpent-2-yn-1-yl)-1H-benzo[d]imidazole-1-carboxamide

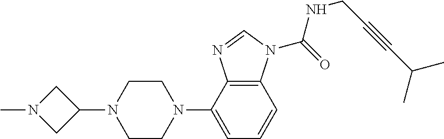 [SMILES: CC(C)C#CCNC(=O)n1cnc2c(N3CCN(C4CN(C)C4)CC3)cccc21]

Following general procedure C, 4-(4-(1-methylazetidin-3-yl)piperazin-1-yl)-1H benzo[d]imidazole (100 mg, 0.37 mmol) and 4-methylpent-2-yn-1-amine (36 mg, 0.37 mmol) afforded the title compound (9.2 mg, 6.3%) as a white solid. ¹H NMR (400 MHz, CDCl₃) δ 8.42 (s, 1H), 7.51 (d, J=8.1 Hz, 1H), 7.29 (t, J=7.6 Hz, 1H), 6.86 (bs, 1H), 6.72 (d, J=7.9 Hz, 1H), 4.27 (d, J=2.9 Hz, 2H), 4.18 (t, J=8.1 Hz, 2H), 3.65-3.41 (m, 6H), 3.37 (t, J=6.8 Hz, 1H), 2.74 (s, 3H), 2.62-2.57 (m, 5H), 1.17 (d, J=6.8 Hz, 6H). LC-MS m/z: 395.1 [M+H]⁺. HPLC Purity (214 nm): 100%; t_{R}=5.90 min.

### Example 300—4-(3-(Methylamino)pyrrolidin-1-yl)-N-(4-methylpent-2-ynyl)-1H-benzo[d]imidazole-1-carboxamide

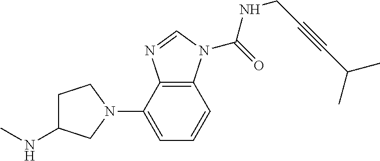 [SMILES: CNC1CCN(c2cccc3c2ncn3C(=O)NCC#CC(C)C)C1]

Following general procedure C, 1-(1H-benzo[d]imidazol-4-yl)-N-methylpyrrolidin-3-amine (200 mg, 0.93 mmol) and 4-methylpent-2-yn-1-amine (72 mg, 0.74 mmol) afforded the title compound (1.4 mg, 0.4%) as a white solid. ¹H NMR (400 MHz, DMSO) δ 8.95 (t, J=5.4 Hz, 1H), 8.49 (s, 1H), 7.34 (d, J=7.9 Hz, 1H), 7.13 (t, J=8.0 Hz, 1H), 6.32 (d, J=8.0 Hz, 1H), 4.12-4.06 (m, 2H), 3.93-3.87 (m, 1H), 3.71 (d, J=7.2 Hz, 1H), 3.65-3.59 (m, 2H), 3.55 (d, J=4.7 Hz, 1H), 3.36 (t, J=5.4 Hz, 1H), 2.63-2.55 (m, 1H), 2.38 (s, 3H), 2.13 (dd, J=12.8, 6.8 Hz, 11H), 1.90-1.83 (m, 1H), 1.12 (d, J=6.9 Hz, 6H). LC-MS m/z: 340.2 [M+H]⁺. HPLC Purity (254 nm): 100%; t_{R}=6.05 min.

### Example 301—4-(5-Methyl-2,5-diazabicyclo[2.2.1]heptan-2-yl)-N-(4-methylpent-2-ynyl)-1H-benzo[d]imidazole-1-carboxamide

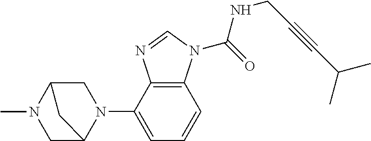 [SMILES: CC(C)C#CCNC(=O)n1cnc2c(N3CC4CC3CN4C)cccc21]

Following general procedure C, 4-(5-methyl-2,5-diazabicyclo[2.2.1]heptan-2-yl)-1H-benzo[d]imidazole (80 mg, 0.35 mmol) and 4-methylpent-2-yn-1-amine (96 mg, 0.7 mmol) afforded the title compound (33.8 mg, 27.5%) as a white solid. ¹H NMR (400 MHz, CDCl₃) δ 8.36 (s, 1H), 7.31 (d, J=8.1 Hz, 1H), 7.23 (d, J=8.2 Hz, 1H), 7.01 (d, J=11.5 Hz, 1H), 6.39 (d, J=7.8 Hz, 1H), 5.62 (bs, 1H), 4.25 (s, 2H), 4.20 (s, 1H), 3.90-3.79 (m, 2H), 3.46 (bs, 1H), 3.18 (bs, 1H), 2.72 (s, 3H), 2.61-2.56 (m, 1H), 2.35-2.20 (m, 2H), 1.18 (d, J=6.9 Hz, 6H). LC-MS m/z: 352.1 [M+H]⁺. HPLC Purity (214 nm): 100%; t_{R}=5.97 min.

### Example 302—4-(Hexahydropyrrolo[1,2-a]pyrazin-2(1H)-yl)-N-(4-methylpent-2-yn-1-yl)-1H-benzo[d]imidazole-1-carboxamide

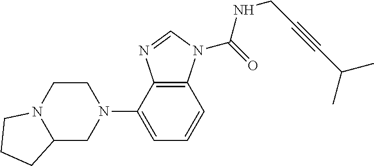 [SMILES: CC(C)C#CCNC(=O)n1cnc2c(N3CCN4CCCC4C3)cccc21]

Following general procedure C, 4-(hexahydropyrrolo[1,2-a]pyrazin-2(1H)-yl)-1H-benzo[d]imidazole (70 mg, 0.4 mmol) and 4-methylpent-2-yn-1-amine (55.0 mg, 0.4 mmol) afforded the title compound (31.2 mg, 21%) as a white solid. ¹H NMR (400 MHz, CDCl₃) δ 8.55 (s, 1H), 7.86 (bs, 1H), 7.64 (d, J=8.2 Hz, 1H), 7.26-7.18 (m, 1H), 6.68 (d, J=8.2 Hz, 1H), 4.24 (d, J=3.0 Hz, 2H), 4.06-4.02 (m, 2H), 3.64-3.28 (m, 5H), 3.16 (bs, 1H), 2.94 (bs, 1H), 2.59-2.53 (m, 1H), 2.18-2.14 (m, 4H), 1.15 (d, J=7.2 Hz, 6H). LC-MS m/z: 366.1 [M+H]⁺. HPLC Purity (214 nm): 100%; t_{R}=6.07 min.

### Example 303—N-(5-Methylhex-3-yn-1-yl)-4-(4-methylpiperazin-1-yl)-1H-benzo[d]imidazole-1-carboxamide

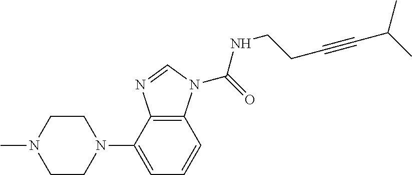 [SMILES: CC(C)C#CCCNC(=O)n1cnc2c(N3CCN(C)CC3)cccc21]

Following general procedure C, 4-(4-methylpiperazin-1-yl)-1H-benzo[d]imidazole (70 mg, 0.32 mmol) and 5-methylhex-3-yn-1-amine (71 mg, 0.47 mmol) afforded the title compound (21.0 mg, 18.8%) as a white solid. ¹H NMR (400 MHz, CDCl₃) δ 7.51 (d, J=8.4 Hz, 1H), 7.30-7.26 (m, 1H), 6.78-6.73 (m, 2H), 5.01 (bs, 1H), 3.69 (bs, 4H), 3.61 (q, J=6.0 Hz, 2H), 3.12 (bs, 4H), 2.63 (s, 3H), 2.58-2.52 (m, 3H), 1.15 (d, J=6.8 Hz, 6H). LC-MS m/z: 354.3 [M+H]⁺. HPLC Purity (214 nm): 100.00%; t_{R}=5.65 min.

### Example 304—4-(5-Methyl-2,5-diazabicyclo[2.2.2]octan-2-yl)-N-(4-methylpent-2-ynyl)-1H-benzo[d]imidazole-1-carboxamide

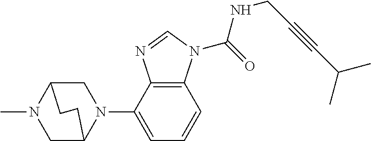 [SMILES: CC(C)C#CCNC(=O)n1cnc2c(N3CC4CCC3CN4C)cccc21]

Following general procedure E, 3-fluoro-2-nitroaniline (322 mg, 2.06 mmol) and 2-methyl-2,5-diazabicyclo[2.2.2]octane (260 mg, 2.06 mmol) afforded 3-(5-methyl-2,5-diazabicyclo[2.2.2]octan-2-yl)-2-nitroaniline (260 mg, 48%) as a yellow oil. LC-MS m/z: 263.2 [M+H]⁺; t_{R}=1.60 min.

Following general procedure F, 3-(5-methyl-2,5-diazabicyclo[2.2.2]octan-2-yl)-2-nitroaniline (260 mg, 1.37 mmol) afforded 3-(5-methyl-2,5-diazabicyclo[2.2.2]octan-2-yl) benzene-1,2-diamine (180 mg, 78%) as a yellow oil. LC-MS m/z: 233.2 [M+H]⁺; t_{R}=1.29 min.

Following general procedure G (method B), 3-(5-methyl-2,5-diazabicyclo[2.2.2]octan-2-yl)benzene-1,2-diamine (180 mg, 0.77 mmol) afforded 2-(1H-benzo[d]imidazol-4-yl)-5-methyl-2,5-diazabicyclo[2.2.2]octane (140 mg, 74%) as a white solid. LC-MS m/z: 243.2 [M+H]⁺. Purity (214 nm): 99%; t_{R}=1.24 min.

Following general procedure C, 2-(1H-benzo[d]imidazol-4-yl)-5-methyl-2,5-diazabicyclo[2.2.2]octane (70 mg, 0.29 mmol) afforded the title compound (25.1 mg, 23.8%) as a yellow solid. ¹H NMR (400 MHz, CDCl₃) δ 8.49 (s, 1H), 7.61 (bs, 1H), 7.44 (d, J=8.1 Hz, 1H), 7.32-7.21 (m, 1H), 6.46 (d, J=8.0 Hz, 1H), 5.50 (s, 1H), 4.31-4.22 (m, 2H), 4.16 (d, J=11.8 Hz, 1H), 3.73 (d, J=11.4 Hz, 1H), 3.61 (d, J=10.3 Hz, 2H), 3.32 (d, J=12.1 Hz, 1H), 2.85 (s, 3H), 2.63-2.39 (m, 2H), 2.19 (d, J=12.1 Hz, 1H), 2.04-1.83 (m, 2H), 1.24-1.08 (m, 6H). LC-MS m/z: 366.4 [M+H]⁺. Purity (214 nm): 100%; t_{R}=5.51 min.

### Example 305—4-(4-Methylpiperazin-1-yl)-N-(pent-2-yn-1-yl)-1H-benzo[d]imidazole-1-carboxamide

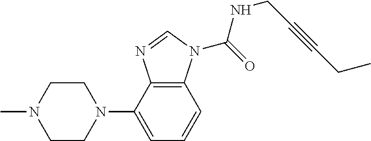 [SMILES: CCC#CCNC(=O)n1cnc2c(N3CCN(C)CC3)cccc21]

Following general procedure C, 4-(4-methylpiperazin-1-yl)-1H-benzo[d]imidazole (150 mg, 0.69 mmol) and pent-2-yn-1-amine (164 mg, 1.38 mmol) afforded the title compound (45.8 mg, 20.1%) as a brown oil. ¹H NMR (400 MHz, CDCl₃) δ 8.46 (s, 1H), 7.51 (d, J=8.0 Hz, 1H), 7.29 (t, J=8.4 Hz, 1H), 6.74 (d, J=7.6 Hz, 1H), 6.66 (t, J=4.4 Hz, 1H), 4.28-4.25 (m, 2H), 3.68 (bs, 4H), 3.12 (bs, 4H), 2.63 (s, 3H), 2.26-2.20 (m, 2H), 1.15 (t, J=7.6 Hz, 3H). LC-MS m/z: 326.2 [M+H]⁺. HPLC Purity (214 nm): 100.00%; t_{R}=5.68 min.

### Example 306—4-(3-(Cyanomethyl)-4-methylpiperazin-1-yl)-N-(4-methylpent-2-ynyl)-1H-benzo[d]imidazole-1-carboxamide

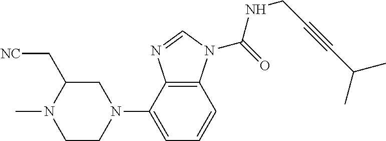 [SMILES: CC(C)C#CCNC(=O)n1cnc2c(N3CCN(C)C(CC#N)C3)cccc21]

Following general procedure E, 3-fluoro-2-nitroaniline (156 mg, 1.0 mmol) and 2-(piperazin-2-yl)acetonitrile (125 mg, 1.0 mmol) afforded 2-(4-(3-amino-2-nitrophenyl)piperazin-2-yl)acetonitrile (90 mg, 34%) as a yellow solid. LC-MS m/z: 262.1 [M+H]⁺; t_{R}=1.44 min.

Following general procedure F, 2-(4-(3-amino-2-nitrophenyl)piperazin-2-yl) acetonitrile (90 mg, 0.34 mmol) afforded 2-(4-(2,3-diaminophenyl)piperazin-2-yl)acetonitrile (70 mg, 88%) as a yellow oil. LC-MS m/z: 232.1 [M+H]⁺; t_{R}=1.16 min.

Following general procedure G (method B), 2-(4-(2,3-diaminophenyl)piperazin-2-yl) acetonitrile (70 mg, 0.3 mmol) afforded 2-(4-(1H-benzo[d]imidazol-4-yl)piperazin-2-yl) acetonitrile (70 mg, 96%) as a yellow oil. LC-MS m/z: 242.2 [M+H]⁺; t_{R}=1.20 min.

To a solution of 2-(4-(1H-benzo[d]imidazol-4-yl)piperazin-2-yl)acetonitrile (70 mg, 0.29 mmol) in MeOH (3 mL) was added HCHO (17 mg, 0.58 mmol) and the mixture was stirred at RT for 1 h. NaBH₃CN (37 mg, 0.58 mmol) was added and the mixture was stirred at RT for 2 h. The reaction mixture was concentrated and the residue was purified by silica gel column chromatography (DCM:MeOH=8:1) to give 2-(4-(1H-benzo[d]imidazol-4-yl)-1-methylpiperazin-2-yl) acetonitrile (70 mg, 95%) as a yellow oil. LC-MS m/z: 256.2 [M+H]⁺; t_{R}=1.43 min.

Following general procedure C, 2-(4-(1H-benzo[d]imidazol-4-yl)-1-methylpiperazin-2-yl)acetonitrile (70 mg, 0.27 mmol) and 4-methylpent-2-yn-1-amine (73 mg, 0.55 mmol) afforded the title compound (3.5 mg, 3.4%) as a white solid. ¹H NMR (400 MHz, CDCl₃) δ 8.34 (s, 1H), 7.40 (d, J=7.7 Hz, 1H), 7.32 (t, J=8.1 Hz, 1H), 6.77 (d, J=7.9 Hz, 1H), 5.84 (bs, 1H), 4.28 (dd, J=5.1, 2.1 Hz, 2H), 4.01 (d, J=11.1 Hz, 1H), 3.79 (d, J=11.4 Hz, 1H), 3.34-3.16 (m, 2H), 3.00-2.76 (m, 3H), 2.76-2.53 (m, 3H), 2.45 (s, 3H), 1.17 (dd, J=13.3, 6.9 Hz, 6H). LC-MS m/z: 379.3 [M+H]⁺. Purity (214 nm): 100%; t_{R}=5.52 min.

### Example 307—N-(4-Methylpent-2-yn-1-yl)-4-(4-(oxetan-3-yl)-piperazin-1-yl)-1H-benzo[d]imidazole-1-carboxamide

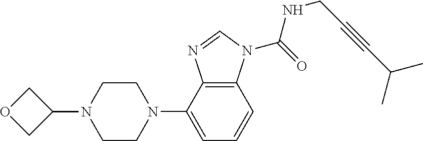 [SMILES: CC(C)C#CCNC(=O)n1cnc2c(N3CCN(C4COC4)CC3)cccc21]

Following general procedure C, 4-(4-(oxetan-3-yl)piperazin-1-yl)-1H-benzo[d]imidazole (80.0 mg, 0.3 mmol) and 4-methylpent-2-yn-1-amine (60.9 mg, 0.4 mmol) afforded the title compound (10.0 mg, 8.7%) as a white solid. ¹H NMR (400 MHz, CDCl₃) 8.34 (s, 1H), 7.39 (d, J=7.7 Hz, 1H), 7.32 (t, J=8.0 Hz, 1H), 6.77 (d, J=7.8 Hz, 1H), 5.86 (bs, 1H), 4.72 (dd, J=9.7, 4.8 Hz, 4H), 4.28 (dd, J=5.1, 2.0 Hz, 2H), 3.79-3.69 (m, 1H), 3.63 (bs, 4H), 2.68 (bs, 4H), 2.63-2.50 (m, 1H), 1.18 (d, J=6.9 Hz, 6H). LC-MS m/z: 382.3 [M+H]⁺. HPLC Purity (254 nm): 100%; t_{R}=5.31 min.

### Example 308—N-(4-Methylpent-2-yn-1-yl)-4-(2-(trifluoromethyl)-aziridin-1-yl)-1H-benzo[d]imidazole-1-carboxamide

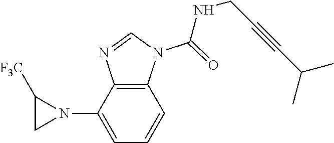 [SMILES: CC(C)C#CCNC(=O)n1cnc2c(N3CC3C(F)(F)F)cccc21]

Following general procedure C, 4-(2-(trifluoromethyl)aziridin-1-yl)-1H-benzo[d]imidazole (10 mg, 0.044 mmol) and 4-methylpent-2-yn-1-amine (12 mg, 0.088 mmol) afforded the title compound (2.0 mg, 13.3%) as a white solid. ¹H NMR (400 MHz, CDCl₃) δ 8.37 (s, 1H), 7.54 (d, J=8.0 Hz, 1H), 7.31 (t, J=8.0 Hz, 1H), 6.98 (d, J=8.0 Hz, 1H), 5.86 (bs, 1H), 4.29-4.28 (m, 2H), 3.01 (bs, 1H), 2.88 (d, J=4.8 Hz, 1H), 2.61-2.57 (m, 1H), 2.53 (d, J=6.4 Hz, 1H), 1.18 (d, J=6.4 Hz, 6H). LC-MS m/z: 351.0 [M+H]⁺. HPLC Purity (214 nm): 100.00%; t_{R}=8.92 min.

### Example 309—4-(4-(2-Fluoroethyl)piperazin-1-yl)-N-(4-methylpent-2-yn-1-yl)-1H-benzo[d]imidazole-1-carboxamide

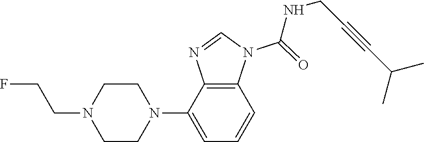 [SMILES: CC(C)C#CCNC(=O)n1cnc2c(N3CCN(CCF)CC3)cccc21]

Following general procedure E, 3-fluoro-2-nitroaniline (500 mg, 3.21 mmol) and 1-(2-fluoroethyl)piperazine hydrochloride (635 mg, 4.81 mmol) afforded 3-(4-(2-fluoroethyl) piperazin-1-yl)-2-nitroaniline (800 mg, 93%) as an orange oil. LC-MS m/z: 269.2 [M+H]⁺. Purity (214 nm): 98%; t_{R}=1.77 min.

Following general procedure F, 3-(4-(2-fluoroethyl)piperazin-1-yl)-2-nitroaniline (800 mg, 2.99 mmol) afford 3-(4-(2-fluoroethyl)piperazin-1-yl)benzene-1,2-diamine (700 mg, 98%) as a brown oil. LC-MS m/z: 239.2 [M+H]⁺. Purity (214 nm): 90%, t_{R}=1.53 min.

Following general procedure G (method B), 3-(4-(2-fluoroethyl)piperazin-1-yl)benzene-1,2-diamine (300 mg, 1.26 mmol) afforded 4-(4-(2-fluoroethyl)piperazin-1-yl)-1H-benzo[d]imidazole (60 mg, 19%) as a yellow oil. LC-MS m/z: 249.1 [M+H]⁺. Purity (214 nm): 98%; t_{R}=1.27 min.

Following general procedure C, 4-(4-(2-fluoroethyl)piperazin-1-yl)-1H-benzo[d]imidazole (60 mg, 0.24 mmol) and 4-methylpent-2-yn-1-amine (64 mg, 0.48 mmol) afforded the title compound (14.9 mg, 17%) as a white solid. ¹H NMR (400 MHz, CDCl₃) δ 8.37 (s, 1H), 7.41 (d, J=8.0 Hz, 1H), 7.31 (t, J=8.1 Hz, 1H), 6.76 (d, J=7.8 Hz, 1H), 6.03 (t, J=4.9 Hz, 1H), 4.72 (t, J=4.8 Hz, 1H), 4.60 (t, J=4.8 Hz, 1H), 4.28 (dd, J=5.1, 2.0 Hz, 2H), 3.60 (bs, 4H), 3.01-2.94 (m, 5H), 2.82 (t, J=5.2 Hz, 1H), 2.59 (dtt, J=13.8, 6.9, 2.0 Hz, 1H), 1.18 (d, J=6.9 Hz, 6H). LC-MS m/z: 372.3 [M+H]⁺. HPLC Purity (214 nm): 98%; t_{R}=5.67 min.

### Example 310—N-(4-Methylpent-2-yn-1-yl)-4-(4-(3,3,3-trifluoropropyl)piperazin-1-yl)-1H-benzo[d]imidazole-1-carboxamide

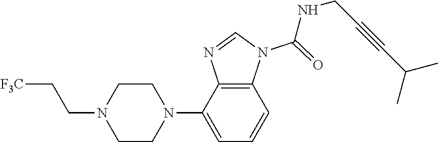 [SMILES: CC(C)C#CCNC(=O)n1cnc2c(N3CCN(CCC(F)(F)F)CC3)cccc21]

Following general procedure C, 4-(4-(3,3,3-trifluoropropyl)piperazin-1-yl)-1H-benzo[d]imidazole (60 mg, 0.20 mmol) and 4-methylpent-2-yn-1-amine (54 mg, 0.40 mmol) afforded the title compound (15.8 mg, 19%) as a white solid. ¹H NMR (400 MHz, CDCl₃) δ 8.34 (s, 1H), 7.37 (d, J=7.6 Hz, 1H), 7.31 (t, J=8.0 Hz, 1H), 6.75 (d, J=8.0 Hz, 1H), 5.84 (t, J=5.1 Hz, 1H), 4.28 (dd, J=5.1, 2.1 Hz, 2H), 3.55 (bs, 4H), 2.81-2.74 (m, 4H), 2.74-2.68 (m, 2H), 2.59 (dtt, J=13.9, 6.9, 1.9 Hz, 1H), 2.45-2.30 (m, 2H), 1.18 (d, J=6.9 Hz, 6H). LC-MS m/z: 422.2 [M+H]⁺. HPLC Purity (214 nm): 99%; t_{R}=6.47 min.

### Example 311—4-(1-Methyl-1,8-diazaspiro[4.5]decan-8-yl)-N-(4-methylpent-2-yn-1-yl)-1H-benzo[d]imidazole-1-carboxamide

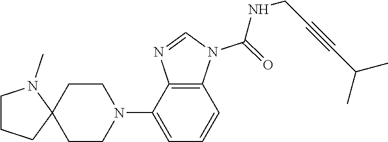 [SMILES: CC(C)C#CCNC(=O)n1cnc2c(N3CCC4(CCCN4C)CC3)cccc21]

Following general procedure E, 1-methyl-1,8-diazaspiro[4.5]decane (400 mg, 2.1 mmol) and 3-fluoro-2-nitroaniline (330 mg, 2.1 mmol) afforded 3-(1-methyl-1,8-diazaspiro[4.5]decan-8-yl)-2-nitroaniline (400 mg, 65.6%) as a yellow solid. LC-MS m/z: 290.36 [M+H]⁺. Purity (214 nm): 95%; t_{R}=1.68 min.

Following general procedure F, 3-(1-methyl-1,8-diazaspiro[4.5]decan-8-yl)-2-nitroaniline (350 mg, 1.21 mmol) afforded 3-(1-methyl-1,8-diazaspiro[4.5]decan-8-yl)benzene-1,2-diamine (280 mg, 88.9%) as a brown solid. LC-MS m/z: 260.38 [M+H]⁺. Purity (214 nm): 94%; t_{R}=1.46 min.

Following general procedure G (method B), 3-(1-methyl-1,8-diazaspiro[4.5]decan-8-yl)benzene-1,2-diamine (280 mg, 1.08 mmol) afforded 4-(1-methyl-1,8-diazaspiro[4.5]decan-8-yl)-1H-benzo[d]imidazole (150 mg, 51.4%) as a black solid. LC-MS m/z: 270.4 [M+H]⁺. Purity (214 nm): 96.55%; t_{R}=1.49 min.

Following general procedure C, 4-(1-methyl-1,8-diazaspiro[4.5]decan-8-yl)-1H-benzo[d]imidazole (70 mg, 0.26 mmol) and 4-methylpent-2-yn-1-amine (50 mg, 0.37 mmol) afforded the title compound (4.6 mg, 4.5%) as a white solid. ¹H NMR (400 MHz, CDCl₃) δ 8.28 (s, 1H), 7.29 (d, J=7.9 Hz, 1H), 7.23 (t, J=8.0 Hz, 1H), 6.70 (d, J=7.7 Hz, 1H), 5.85 (bs, 1H), 4.21 (dd, J=5.1, 2.0 Hz, 2H), 4.14 (d, J=12.0 Hz, 2H), 2.91-2.85 (m, 4H), 2.55-2.49 (m, 1H), 2.33 (s, 3H), 2.02 (td, J=12.7, 4.3 Hz, 2H), 1.80 (bs, 4H), 1.41 (d, J=12.6 Hz, 2H), 1.11 (dd, J=14.0, 6.9 Hz, 6H). LC-MS m/z: 393.5 [M+H]⁺. HPLC Purity (214 nm): 92.93%; t_{R}=6.25 min.

### Example 312—4-(2-Methyloctahydropyrrolo[3,4-d]azepin-6(1H)-yl)-N-(4-methylpent-2-yn-1-yl)-1H-benzo[d]imidazole-1-carboxamide

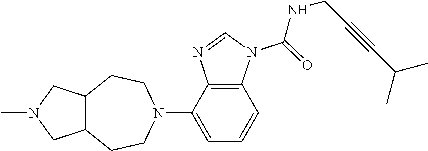 [SMILES: CC(C)C#CCNC(=O)n1cnc2c(N3CCC4CN(C)CC4CC3)cccc21]

Following general procedure E, 3-fluoro-2-nitroaniline (150 mg, 1.0 mmol) and 2-methyldecahydro-pyrrolo[3,4-d]azepine (150 mg, 1.0 mmol) afforded 3-(2-methyloctahydro pyrrolo[3,4-d]azepin-6(7H)-yl)-2-nitroaniline (150 mg, 54%) as a yellow solid. LC-MS m/z: 291.2 [M+H]⁺; t_{R}=1.53 min.

Following general procedure F, 3-(2-methyloctahydropyrrolo[3,4-d]azepin-6(7H)-yl)-2-nitroaniline (340 mg, 1.37 mmol) afforded 3-(2-methyloctahydropyrrolo[3,4-d]azepin-6(7H)-yl)benzene-1,2-diamine (130 mg, 97%) as a yellow oil. LC-MS m/z: 261.2 [M+H]⁺; t_{R}=1.21 min.

Following general procedure G (method B), 3-(2-methyloctahydropyrrolo[3,4-d]azepin-6(7H)-yl)benzene-1,2-diamine (130 mg, 0.5 mmol) afforded 6-(1H-benzo[d]imidazol-4-yl)-2-methyldecahydropyrrolo[3,4-d]azepine (110 mg, 81%) as a yellow oil. LC-MS m/z: 271.2 [M+H]⁺. Purity (214 nm): >99%; t_{R}=1.50 min.

Following general procedure C, 6-(1H-benzo[d]imidazol-4-yl)-2-methyldecahydro pyrrolo[3,4-d](110 mg, 0.41 mmol) and 4-methylpent-2-yn-1-amine (81 mg, 0.61 mmol) afforded the title compound (9.4 mg, 5.9%) as a white solid. ¹H NMR (400 MHz, CDCl₃) δ 8.41 (s, 1H), 7.38 (d, J=7.9 Hz, 1H), 7.23 (d, J=8.1 Hz, 1H), 6.95 (bs, 1H), 6.62 (d, J=8.0 Hz, 1H), 4.39-4.19 (m, 4H), 3.62 (bs, 2H), 3.36-3.20 (m, 2H), 2.89 (bs, 2H), 2.69 (s, 3H), 2.63-2.47 (m, 3H), 1.93 (bs, 4H), 1.16 (dd, J=10.3, 6.9 Hz, 6H). LC-MS m/z: 394.3 [M+H]⁺. HPLC Purity (214 nm): 100%; t_{R}=5.41 min.

### Example 313—4-(1-Methyl-1,7-diazaspiro[3.5]nonan-7-yl)-N-(4-methylpent-2-yn-1-yl)-1H-benzo[d]imidazole-1-carboxamide

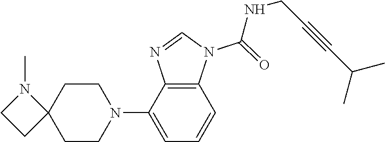 [SMILES: CC(C)C#CCNC(=O)n1cnc2c(N3CCC4(CC3)CCN4C)cccc21]

Following general procedure E, 3-fluoro-2-nitroaniline (468 mg, 3.0 mmol) and 1-methyl-1,6-diazaspiro[3.3]heptane (420 mg, 3.0 mmol) afforded 3-(1-methyl-1,7-diazaspiro[3.5]nonan-7-yl)-2-nitroaniline (552 mg, 66%) as a yellow solid. LC-MS m/z: 277.2 [M+H]⁺. Purity (214 nm): 96%; t_{R}=1.61 min.

Following general procedure F, 3-(1-methyl-1,7-diazaspiro[3.5]nonan-7-yl)-2-nitroaniline (552 mg, 2.0 mmol) afforded 3-(1-methyl-1,7-diazaspiro[3.5]nonan-7-yl)benzene-1,2-diamine (492 mg, 100%) as a yellow oil. LC-MS m/z: 247.2 [M+H]⁺. Purity (214 nm): 85%; t_{R}=1.45 min.

Following general procedure G (method A), 3-(1-methyl-1,7-diazaspiro[3.5]nonan-7-yl)benzene-1,2-diamine (492 mg, 2.0 mmol) afforded 7-(1H-benzo[d]imidazol-4-yl)-1-methyl-1,7-diazaspiro[3.5]nonane (282 mg, 55%) as a yellow oil. LC-MS m/z: 257.3 [M+H]⁺. Purity (214 nm): 76%; t_{R}=1.31 min.

Following general procedure C, 7-(1H-benzo[d]imidazol-4-yl)-1-methyl-1,7-diazaspiro[3.5]nonane (102 mg, 0.4 mmol) and 4-methylpent-2-yn-1-amine (67.0 mg, 0.5 mmol) afforded the title compound (3.1 mg, 2%) as a yellow oil. ¹H NMR (400 MHz, CDCl₃) δ 8.40 (s, 1H), 7.47 (d, J=8.1 Hz, 1H), 7.35-7.26 (m, 1H), 6.72 (d, J=7.9 Hz, 1H), 6.56 (bs, 1H), 4.27 (d, J=3.1 Hz, 2H), 4.16 (d, J=11.1 Hz, 2H), 3.82 (bs, 2H), 2.87 (t, J=11.7 Hz, 2H), 2.61-2.57 (m, 1H), 2.55 (s, 3H), 2.41-2.36 (m, 2H), 2.26-2.16 (m, 4H), 1.18 (d, J=6.9 Hz, 6H). LC-MS m/z: 380.3 [M+H]⁺. HPLC Purity (214 nm): 98.47%; t_{R}=6.05 min.

### Example 314—4-(3-(4-Methylpiperazin-1-yl)azetidin-1-yl)-N-(2-phenoxyethyl)-1H-benzo[d]imidazole-1-carboxamide

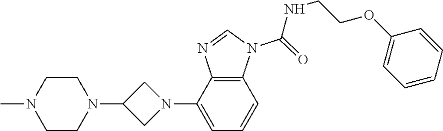 [SMILES: CN1CCN(C2CN(c3cccc4c3ncn4C(=O)NCCOc3ccccc3)C2)CC1]

Following general procedure C, 4-(3-(4-methylpiperazin-1-yl)azetidin-1-yl)-1H-benzo[d]imidazole (120 mg, 0.44 mmol) and 2-phenoxyethanamine (60 mg, 0.44 mmol) afforded the title compound (84.1 mg, 43.8%) as a white solid. ¹H NMR (400 MHz, CDCl₃) δ 8.33 (s, 1H), 7.34 (dd, J=8.6, 7.5 Hz, 2H), 7.22 (t, J=8.0 Hz, 1H), 7.11-6.90 (m, 4H), 6.49 (t, J=5.3 Hz, 1H), 6.28 (d, J=7.8 Hz, 1H), 4.38 (t, J=7.4 Hz, 2H), 4.25 (t, J=5.0 Hz, 2H), 4.07 (dd, J=7.9, 5.4 Hz, 2H), 3.94 (q, J=5.0 Hz, 2H), 3.52-3.36 (m, 3H), 2.89 (bs, 2H), 2.66 (bs, 4H), 2.55 (s, 3H). LC-MS m/z: 435.2 [M+H]⁺. HPLC Purity (254 nm): 100%; t_{R}=6.02 min.

### Example 315—4-(3-(Methylamino)pyrrolidin-1-yl)-N-(2-phenoxyethyl)-1H-benzo[d]imidazole-1-carboxamide

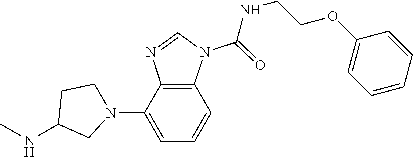 [SMILES: CNC1CCN(c2cccc3c2ncn3C(=O)NCCOc2ccccc2)C1]

Following general procedure C, 1-(1H-benzo[d]imidazole-4-yl)-N-methylpyrrolidin-3-amine (300 mg, 1.4 mmol) and 2-phenoxyethanamine (95 mg, 0.7 mmol) afforded the title compound (15.8 mg, 3.0%) as a white solid. ¹H NMR (400 MHz, DMSO-dh) δ 8.77 (t, J=5.5 Hz, 1H), 8.50 (s, 1H), 7.44-7.19 (m, 3H), 7.12 (t, J=8.1 Hz, 1H), 6.97 (dd, J=14.0, 7.6 Hz, 3H), 6.31 (d, J=7.8 Hz, 1H), 4.18 (t, J=5.6 Hz, 2H), 3.88 (dd, J=10.7, 6.4 Hz, 1H), 3.69 (dt, J=11.1, 5.6 Hz, 2H), 3.62-3.50 (m, 2H), 3.42-3.30 (m, 2H), 2.37 (s, 3H), 2.12 (dd, J=12.4, 6.9 Hz, 1H), 1.84 (dd, J=12.5, 6.2 Hz, 1H). LC-MS m/z: 380.3 [M+H]⁺. HPLC Purity (254 nm): 100%; t_{R}=6.06 min.

### Example 316—4-(Hexahydropyrrolo[1,2-a]pyrazin-2(1H)-yl)-N-(2-phenoxyethyl)-1H-benzo[d]imidazole-1-carboxamide

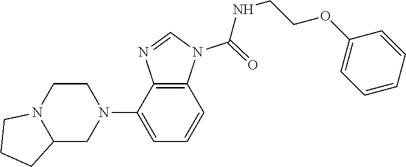 [SMILES: O=C(NCCOc1ccccc1)n1cnc2c(N3CCN4CCCC4C3)cccc21]

Following general procedure C, 4-(hexahydropyrrolo[1,2-a]pyrazin-2(1H)-yl)-1H-benzo[d]imidazole (80.0 mg, 0.3 mmol) and 2-phenoxyethanamine (50.4, 0.4 mmol) afforded the title compound (69.0 mg, 51.5%) as a white solid. ¹H NMR (400 MHz, CDCl₃)) δ 8.40 (s, 1H), 7.36 (t, J=8.0 Hz, 1H), 7.32-7.26 (m, 3H), 7.00 (t, J=7.4 Hz, 1H), 6.94 (d, J=7.8 Hz, 2H), 6.76 (d, J=7.8 Hz, 1H), 6.55 (bs, 1H), 4.24 (t, J=5.0 Hz, 3H), 4.10 (d, J=11.1 Hz, 1H), 3.94 (dd, J=10.3, 5.3 Hz, 2H), 3.46-3.26 (m, 3H), 3.07 (s, 1H), 2.89 (bs, 2H), 2.61 (bs, 1H), 2.14-2.00 (m, 2H), 1.94 (s, 1H), 1.80 (s, 1H). LC-MS m/z: 406.2 [M+H]⁺. HPLC Purity (214 nm): 98.73%; t_{R}=8.25 min.

### Example 317—4-(3-(Cyanomethyl)-4-methylpiperazin-1-yl)-N-(2-phenoxyethyl)-1H-benzo[d]imidazole-1-carboxamide

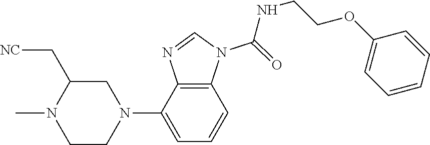 [SMILES: CN1CCN(c2cccc3c2ncn3C(=O)NCCOc2ccccc2)CC1CC#N]

Following general procedure C, 2-(4-(1H-benzo[d]imidazol-4-yl)-1-methylpiperazin-2-yl)acetonitrile (30 mg, 0.12 mmol) and 2-phenoxyethanamine (16 mg, 0.12 mmol) afforded the title compound (8.3 mg, 16.9%) as a white solid. ¹H NMR (400 MHz, CDCl₃) δ 8.37 (s, 1H), 7.32 (ddd, J=14.9, 11.7, 7.6 Hz, 4H), 7.01 (t, J=7.4 Hz, 1H), 6.94 (d, J=8.2 Hz, 2H), 6.75 (d, J=7.7 Hz, 1H), 6.32 (bs, 1H), 4.24 (t, J=4.9 Hz, 2H), 4.03 (d, J=12.0 Hz, 1H), 3.96 (q, J=8.4 Hz, 2H), 3.78 (d, J=11.8 Hz, 1H), 3.34-3.18 (m, 2H), 2.96 (d, J=12.5 Hz, 1H), 2.91-2.74 (m, 2H), 2.74-2.58 (m, 2H), 2.44 (s, 3H). LC-MS m/z: 419.2 [M+H]⁺. Purity (214 nm): 96.59%; t_{R}=5.45 min.

### Example 318—N-(2-(3,3-Difluorocyclopentyl)ethyl)-4-(3-(4-methylpiperazin-1-yl)azetidin-1-yl)-1H-benzo[d]imidazole-1-carboxamide

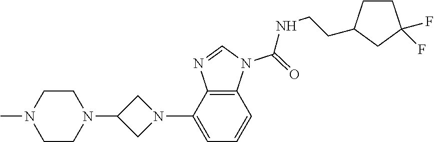 [SMILES: CN1CCN(C2CN(c3cccc4c3ncn4C(=O)NCCC3CCC(F)(F)C3)C2)CC1]

Following general procedure C, 4 4-(3-(4-methylpiperazin-1-yl)azetidin-1-yl)-1H-benzo[d]imidazole (120 mg, 0.44 mmol) and 2-(3,3-difluorocyclopentyl)ethan-1-amine (65 mg, 0.44 mmol) afforded the title compound (43.6 mg, 22.7%) as a white solid. ¹H NMR (400 MHz, CDCl₃) δ 8.31 (s, 1H), 7.21 (t, J=8.0 Hz, 1H), 7.07 (d, J=8.0 Hz, 1H), 6.30 (bs, 1H), 6.27 (d, J=6.2 Hz, 1H), 4.36 (t, J=7.3 Hz, 2H), 4.11-3.92 (m, 2H), 3.50 (dd, J=14.2, 6.4 Hz, 3H), 3.46-3.38 (m, 3H), 2.91 (bs, 2H), 2.66 (bs, 3H), 2.54 (s, 3H), 2.44-2.27 (m, 1H), 2.26-2.11 (m, 2H), 2.11-2.02 (m, 2H), 1.86-1.76 (m, 3H), 1.56-1.46 (m, 1H). LC-MS m/z: 447.2 [M+H]⁺. HPLC Purity (214 nm): 98.28%; t_{R}=6.15 min.

### Examples 319a and 319b—6-Fluoro-2-methoxy-5-(4-(1-methylazetidin-3-yl)piperazin-1-yl)-N-(3-phenylpropyl)-1H-benzo[d]imidazole-1-carboxamide and 5-fluoro-2-methoxy-6-(4-(1-methylazetidin-3-yl)piperazin-1-yl)-N-(3-phenylpropyl)-1H-benzo[d]imidazole-1-carboxamide

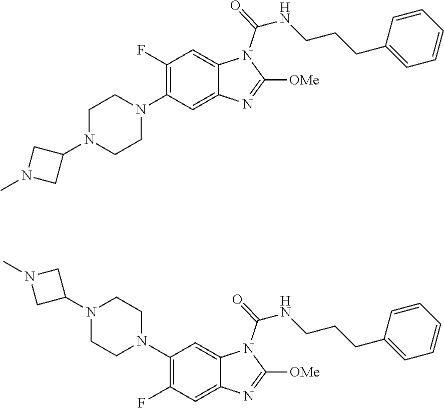 [SMILES: COc1nc2cc(N3CCN(C4CN(C)C4)CC3)c(F)cc2n1C(=O)NCCCc1ccccc1 ; COc1nc2cc(F)c(N3CCN(C4CN(C)C4)CC3)cc2n1C(=O)NCCCc1ccccc1]

Following general procedure E, tert-butyl piperazine-1-carboxylate (10 g, 54 mmol) afforded tert-butyl 4-(5-amino-2-fluoro-4-nitrophenyl)piperazine-1-carboxylate (10 g, 55.6%) as a yellow solid. LC-MS m/z: 285.1 [M-56+H]⁺. Purity (214 nm): 100%; t_{R}=1.66 min.

Following general procedure F, tert-butyl 4-(5-amino-2-fluoro-4-nitrophenyl)piperazine-1-carboxylate (10 g, 29.4 mmol) afforded tert-butyl 4-(4,5-diamino-2-fluorophenyl)piperazine-1-carboxylate (5.0 g, 50%) as a red oil. LC-MS m/z: 311.2 [M+H]⁺. Purity (214 nm): 89.47%; t_{R}=1.60 min.

Following general procedure G (method A), tert-butyl 4-(4,5-diamino-2-fluorophenyl)piperazine-1-carboxylate (5.0 g, 16 mmol) afforded tert-butyl 4-(5-fluoro-2-methoxy-1H-benzo[d]imidazol-6-yl)piperazine-1-carboxylate (4.8 g, 82.7%) as a red solid. LC-MS m/z: 351.2 [M+H]⁺. Purity (254 nm): 75.11%; t_{R}=1.69 min.

A solution of 4-(5-fluoro-2-methoxy-1H-benzo[d]imidazol-6-yl)piperazine-1-carboxylate (4.8 g, 13.7 mmol) in TFA was stirred at RT for 1 h. Then, the reaction mixture was concentrated to afford a residue which was purified by silica gel chromatography (DCM:MeOH; 10:1) to give 5-fluoro-2-methoxy-6-(piperazin-1-yl)-1H-benzo[d]imidazole (5.8 mg, 100%) as a red solid. LC-MS m/z: 251.1 [M+H]⁺, t_{R}=1.08 min.

To a solution 5-fluoro-2-methoxy-6-(piperazin-1-yl)-1H-benzo[d]imidazole (5.8 g, 23 mmol) in MeOH (100 mL) was added tert-butyl 3-oxoazetidine-1-carboxylate (5.5 g, 32.2 mmol). The mixture was stirred at RT for 30 min and then NaBH₃CN (4.3 g, 69 mmol) was added and the mixture was stirred overnight. The mixture was concentrated and purified by silica gel column chromatography (DCM:MeOH=10:1) to give 5-fluoro-2-methoxy-6-(4-(1-methylazetidin-3-yl)piperazin-1-yl)-1H-benzo[d]imidazole (4.1 g, 55.4%) as a yellow solid. LC-MS m/z: 320.2 [M+H]⁺. Purity (214 nm): 86.61%; t_{R}=1.21 min.

Following general procedure C, 5-fluoro-2-methoxy-6-(4-(1-methylazetidin-3-yl)piperazin-1-yl)-1H-benzo[d]imidazole (108 mg, 0.34 mmol) and 3-phenylpropan-1-amine (46 mg, 0.34 mmol) afforded 6-fluoro-2-methoxy-5-(4-(1-methylazetidin-3-yl)piperazin-1-yl)-N-(3-phenylpropyl)-1H-benzo[d]imidazole-1-carboxamide (21.9 mg, 13.4%) and 5-fluoro-2-methoxy-6-(4-(1-methylazetidin-3-yl)piperazin-1-yl)-N-(3-phenylpropyl)-1H-benzo[d]imidazole-1-carboxamide (11.2 mg, 6.9%) both as white solids.

6-fluoro-2-methoxy-5-(4-(1-methylazetidin-3-yl)piperazin-1-yl)-N-(3-phenyl propyl)-1H-benzo[d]imidazole-1-carboxamide: ¹H NMR (400 MHz, CDCl₃) δ 7.90 (d, J=12.6 Hz, 1H), 7.29 (t, J=6.8 Hz, 2H), 7.21 (d, J=6.3 Hz, 3H), 7.10 (d, J=7.6 Hz, 1H), 6.89 (t, J=5.4 Hz, 1H), 4.28 (s, 3H), 4.20 (t, J=7.5 Hz, 2H), 3.59-3.50 (m, 2H), 3.50-3.43 (m, 4H), 3.42-3.31 (m, 1H), 3.10 (bs, 4H), 2.75 (t, J=7.2 Hz, 2H), 2.73 (s, 3H), 2.53 (bs, 4H), 2.07-1.93 (m, 2H). LC-MS m/z: 481.1 [M+H]⁺. HPLC Purity (214 nm): 100%; t_{R}=6.37 min.

5-fluoro-2-methoxy-6-(4-(1-methylazetidin-3-yl)piperazin-1-yl)-N-(3-phenyl propyl)-1H-benzo[d]imidazole-1-carboxamide: ¹H NMR (400 MHz, CDCl₃) δ 7.90 (d, J=12.6 Hz, 1H), 7.29 (t, J=6.8 Hz, 2H), 7.21 (d, J=6.3 Hz, 3H), 7.10 (d, J=7.6 Hz, 1H), 6.89 (t, J=5.4 Hz, 1H), 4.28 (s, 3H), 4.20 (t, J=7.5 Hz, 2H), 3.59-3.50 (m, 2H), 3.51-3.43 (m, 4H), 3.42-3.31 (m, 1H), 3.10 (bs, 4H), 2.75 (t, J=7.6 Hz, 2H), 2.72 (t, J=7.2 Hz, 3H), 2.53 (bs, 4H), 2.07-1.98 (m, 2H). LC-MS m/z: 481.1 [M+H]⁺. HPLC Purity (214 nm): 96.24%; t_{R}=6.16 min.

### Examples 320a and 320b—6-Fluoro-2-methoxy-5-(4-(1-methylazetidin-3-yl)piperazin-1-yl)-N-(4-methylpent-2-yn-1-yl)-1H-benzo[d]imidazole-1-carboxamide and 5-fluoro-2-methoxy-6-(4-(1-methylazetidin-3-yl)piperazin-1-yl)-N-(4-methylpent-2-yn-1-yl)-1H-benzo[d]imidazole-1-carboxamide

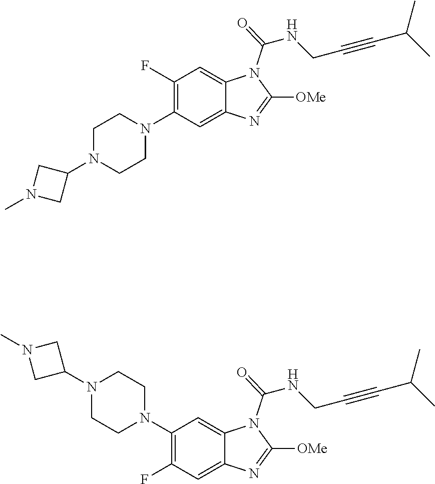

Following general procedure C, 5-fluoro-2-methoxy-6-(4-(1-methylazetidin-3-yl)piperazin-1-yl)-1H-benzo[d]imidazole (108 mg, 0.34 mmol) and 4-methylpent-2-yn-1-amine (46 mg, 0.34 mmol) afforded 6-fluoro-2-methoxy-5-(4-(1-methylazetidin-3-yl)piperazin-1-yl)-N-(4-methylpent-2-yn-1-yl)-1H-benzo[d]imidazole-1-carboxamide (11.8 mg, 8.5%) and 5-fluoro-2-methoxy-6-(4-(1-methylazetidin-3-yl)piperazin-1-yl)-N-(4-methylpent-2-yn-1-yl)-1H-benzo[d]imidazole-1-carboxamide (4.7 mg, 3.4%) both as white solids.

6-fluoro-2-methoxy-5-(4-(1-methylazetidin-3-yl)piperazin-1-yl)-N-(4-methylpent-2-yn-1-yl)-1H-benzo[d]imidazole-1-carboxamide: ¹H NMR (400 MHz, CDCl₃) δ 7.90 (d, J=12.5 Hz, 1H), 7.11 (d, J=7.7 Hz, 1H), 7.03 (t, J=5.2 Hz, 1H), 4.30 (d, J=10.3 Hz, 3H), 4.30 (s, 3H), 4.26-4.09 (m, 4H), 3.46 (t, J=8.2 Hz, 2H), 3.41-3.29 (m, 1H), 3.10 (bs, 4H), 2.72 (s, 3H), 2.63-2.53 (m, 5H), 1.16 (d, J=6.8 Hz, 6H). LC-MS m/z: 443.1 [M+H]⁺. HPLC Purity (214 nm): 100%; t_{R}=6.14 min.

5-fluoro-2-methoxy-6-(4-(1-methylazetidin-3-yl)piperazin-1-yl)-N-(4-methylpent-2-yn-1-yl)-1H-benzo[d]imidazole-1-carboxamide: ¹H NMR (400 MHz, CDCl₃) δ 7.86 (d, J=8.0 Hz, 1H), 7.19 (d, J=11.9 Hz, 1H), 7.10 (t, J=5.1 Hz, 1H), 4.32 (s, 3H), 4.26-4.18 (m, 4H), 3.50 (t, J=8.0 Hz, 2H), 3.39-3.35 (m, 1H), 3.12 (bs, 4H), 2.74 (s, 3H), 2.64-2.49 (m, 5H), 1.18 (d, J=6.8 Hz, 6H). LC-MS m/z: 443.1 [M+H]⁺. HPLC Purity (214 nm): 78.99%; t_{R}=6.52 min.

### Examples 321a and 321b—6-Fluoro-2-methoxy-N-(4-methylpent-2-yn-1-yl)-5-morpholino-1H-benzo[d]imidazole-1-carboxamide and 5-fluoro-2-methoxy-N-(4-methyl pent-2-yn-1-yl)-6-morpholino-1H-benzo[d]imidazole-1-carboxamide

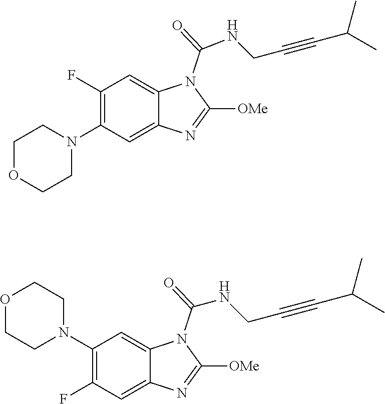 [SMILES: COc1nc2cc(N3CCOCC3)c(F)cc2n1C(=O)NCC#CC(C)C ; COc1nc2cc(F)c(N3CCOCC3)cc2n1C(=O)NCC#CC(C)C]

Following general procedure C, 4-(6-fluoro-2-methoxy-1H-benzo-[d]imidazol-5-yl)morpholine (100 mg, 0.40 mmol) and 4-phenylbutan-1-amine (85 mg, 0.64 mmol) afforded 6-fluoro-2-methoxy-N-(4-methylpent-2-yn-1-yl)-5-morpholino-1H-benzo[d]imidazole-1-carboxamide (37.8 mg, 25%) and 5-fluoro-2-methoxy-N-(4-methylpent-2-yn-1-yl)-6-morpholino-1H-benzo[d]imidazole-1-carboxamide (29.5 mg, 20%) both as white solids.

6-fluoro-2-methoxy-N-(4-methylpent-2-yn-1-yl)-5-morpholino-1H-benzo[d]imidazole-1-carboxamide: ¹H NMR (400 MHz, CDCl₃) δ 7.91 (d, J=12.6 Hz, 1H), 7.11 (d, J=7.7 Hz, 1H), 7.05 (t, J=5.0 Hz, 1H), 4.32 (s, 3H), 4.20 (dd, J=5.2, 2.1 Hz, 2H), 3.93-3.87 (m, 4H), 3.11-3.03 (m, 4H), 2.58 (dtd, J=8.9, 6.9, 4.9 Hz, 1H), 1.18 (d, J=6.9 Hz, 6H). LC-MS m/z: 375.0 [M+H]⁺. HPLC Purity (214 nm): 99%; t_{R}=8.89 min.

5-fluoro-2-methoxy-N-(4-methylpent-2-yn-1-yl)-6-morpholino-1H-benzo[d]imidazole-1-carboxamide: ¹H NMR (400 MHz, CDCl₃) δ 7.87 (d, J=8.0 Hz, 1H), 7.20 (d, J=12.0 Hz, 1H), 7.10 (t, J=4.7 Hz, 1H), 4.32 (s, 3H), 4.21 (dd, J=5.2, 2.1 Hz, 2H), 3.93-3.85 (m, 4H), 3.13-3.05 (m, 4H), 2.64-2.52 (m, 1H), 1.18 (d, J=6.9 Hz, 6H). LC-MS m/z: 375.0 [M+H]⁺. HPLC Purity (214 nm): 99%; t_{R}=8.88 min.

### Example 322—2-Methoxy-4-(4-methylpiperazin-1-yl)-N-(3-phenylprop-2-yn-1-yl)-1H-benzo[d]imidazole-1-carboxamide

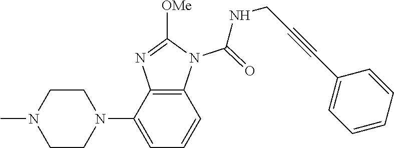 [SMILES: COc1nc2c(N3CCN(C)CC3)cccc2n1C(=O)NCC#Cc1ccccc1]

Following general procedure C, 2-methoxy-4-(4-methylpiperazin-1-yl)-1H-benzo[d]imidazole (98.0 mg, 0.4 mmol) and 3-phenylprop-2-yn-1-amine (45.0 mg, 0.4 mmol) afforded the title compound (6.7 mg, 5%) as a white solid. ¹H NMR (400 MHz, CDCl₃) δ 7.86 (d, J=8.3 Hz, 1H), 7.50-7.40 (m, 2H), 7.33-7.30 (m, 3H), 7.16 (t, J=8.1 Hz, 1H), 6.74 (d, J=7.9 Hz, 1H), 4.47 (d, J=5.2 Hz, 2H), 4.34 (s, 3H), 3.60 (bs, 4H), 3.06 (bs, 4H), 2.60 (s, 3H). LC-MS m/z: 404.2 [M+H]⁺. HPLC Purity (214 nm): 94.60%; t_{R}=7.02 min.

### Example 323—N-(4-Methylbenzyl)-4-(4-methylpiperazin-1-yl)-1H-benzo[d]imidazole-1-carboxamide

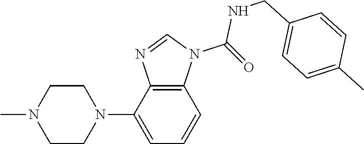

Following general procedure C, 4-(4-methylpiperazin-1-yl)-1H-benzo[d]imidazole (100 mg, 0.46 mmol) and p-tolylmethanamine (67 mg, 0.55 mmol) afforded the title compound (60.0 mg, 36.1%) as a white solid. ¹H NMR (400 MHz, CDCl₃) δ 8.50 (s, 1H), 7.48 (d, J=8.4 Hz, 1H), 7.31-7.26 (m, 3H), 7.25-7.18 (m, 2H), 6.97 (bs, 1H), 6.70 (d, J=7.6 Hz, 1H), 4.63 (d, J=5.2 Hz, 2H), 3.67 (bs, 4H), 3.15 (bs, 4H), 2.63 (s, 3H), 2.35 (s, 3H). LC-MS m/z: 364.1 [M+H]⁺. HPLC Purity (214 nm): 100.0%; t_{R}=5.46 min.

### Example 324—4-(4-Methylpiperazin-1-yl)-N-phenethyl-1H-benzo[d]imidazole-1-carboxamide

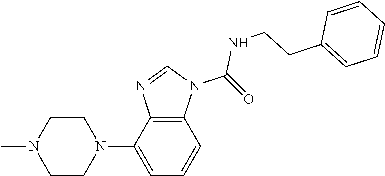 [SMILES: CN1CCN(c2cccc3c2ncn3C(=O)NCCc2ccccc2)CC1]

Following general procedure C, 4-(4-methylpiperazin-1-yl)-1H-benzo[d]imidazole (100 mg, 0.46 mmol) and 2-phenylethan-1-amine (84 mg, 0.69 mmol) afforded the title compound (56.3 mg, 33.7%) as a yellow solid. ¹H NMR (400 MHz, CDCl₃) δ 8.40 (s, 1H), 7.36-7.33 (m, 2H), 7.29-7.26 (m, 3H), 7.26-7.20 (m, 2H), 6.70 (d, J=4.8 Hz, 1H), 6.52 (bs, 1H), 3.77 (q, J=5.2 Hz, 2H), 3.67 (bs, 4H), 3.13 (bs, 4H), 3.01 (t, J=5.6 Hz, 2H), 2.63 (s, 3H). LC-MS m/z: 364.2 [M+H]⁺. HPLC Purity (214 nm): 98.75%; t_{R}=5.95 min.

### Example 325—4-(4-(1-Methylazetidin-3-yl)piperazin-1-yl)-N-phenethyl-1H-benzo[d]imidazole-1-carboxamide

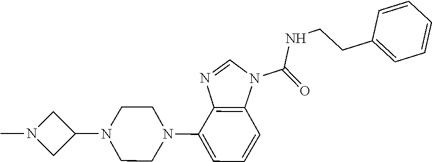 [SMILES: CN1CC(N2CCN(c3cccc4c3ncn4C(=O)NCCc3ccccc3)CC2)C1]

Following general procedure B, 4-(4-(1-methylazetidin-3-yl)piperazin-1-yl)-1H-benzo[d]imidazole (123 mg, 0.45 mmol) and (2-isocyanatoethyl)benzene (85 mg, 0.58 mmol) afforded the title compound (4.1 mg, 2.2%) as a brown solid. ¹H NMR (400 MHz, CDCl₃) δ 8.30 (s, 1H), 7.40-7.35 (m, 2H), 7.32-7.27 (m, 3H), 7.19 (t, J=8.1 Hz, 1H), 7.10 (d, J=7.9 Hz, 1H), 6.69 (d, J=7.7 Hz, 1H), 6.19 (bs, 1H), 4.06 (t, J=7.6 Hz, 2H), 3.78 (dd, J=12.6, 6.7 Hz, 2H), 3.53 (bs, 4H), 3.45-3.39 (m, 2H), 3.35-3.30 (m, 1H), 3.02 (t, J=6.8 Hz, 2H), 2.66 (s, 3H), 2.61-2.57 (m, 4H). LC-MS m/z: 419.3 [M+H]⁺. HPLC Purity (214 nm): 100%; t_{R}=6.47 min.

### Example 326—4-(4-(Oxetan-3-yl)piperazin-1-yl)-N-phenethyl-1H-benzo[d]imidazole-1-carboxamide

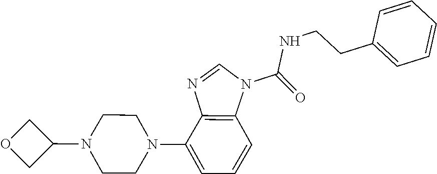 [SMILES: O=C(NCCc1ccccc1)n1cnc2c(N3CCN(C4COC4)CC3)cccc21]

Following general procedure B, 4-(4-(oxetan-3-yl)piperazin-1-yl)-1H-benzo[d]imidazole (100.0 mg, 0.4 mmol) and (2-isocyanatoethyl)benzene (90.1 mg, 0.6 mmol) afforded the title compound (16.4 mg, 10.1%) as a white solid. ¹H NMR (400 MHz, CDCl₃) δ 8.27 (s, 1H), 7.39-7.33 (m, 2H), 7.32-7.27 (m, 3H), 7.19 (t, J=8.1 Hz, 1H), 6.99 (d, J=8.2 Hz, 1H), 6.71 (d, J=7.9 Hz, 1H), 5.71 (bs, 1H), 4.67 (p, J=6.4 Hz, 4H), 3.80 (dd, J=12.6, 6.5 Hz, 2H), 3.65-3.49 (m, 5H), 3.02 (t, J=6.8 Hz, 2H), 2.65-2.58 (m, 4H). LC-MS m/z: 406.2 [M+H]⁺. HPLC Purity (214 nm): 99.20%; t_{R}=5.33 min.

### Example 327—4-(4-(Oxetan-3-yl)piperazin-1-yl)-N-(2-phenoxyethyl)-1H-benzo[d]imidazole-1-carboxamide

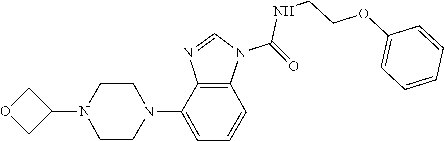 [SMILES: O=C(NCCOc1ccccc1)n1cnc2c(N3CCN(C4COC4)CC3)cccc21]

Following general procedure C, 4-(4-(oxetan-3-yl)piperazin-1-yl)-1H-benzo[d]imidazole (100.0 mg, 0.4 mmol) and 2-phenoxyethanamine (60.0 mg, 0.4 mmol) afforded the title compound (5.5 mg, 3.3%) as a white solid. ¹H NMR (400 MHz, CDCl₃) δ 8.36 (s, 1H), 7.35-7.27 (m, 4H), 7.00 (dd, J=9.0, 5.4 Hz, 1H), 6.94 (d, J=8.3 Hz, 2H), 6.75 (d, J=7.5 Hz, 1H), 6.30 (bs, 1H), 4.77-4.63 (m, 4H), 4.24 (t, J=5.0 Hz, 2H), 3.94 (dd, J=10.3, 5.2 Hz, 2H), 3.69-3.59 (m, 5H), 2.68-2.56 (m, 4H). LC-MS m/z: 422.2 [M+H]⁺. HPLC Purity (214 nm): 98.05%; t_{R}=5.28 min.

### Example 328—4-(5-(1-Methylazetidin-3-yl)-2,5-diazabicyclo[2.2.1]heptan-2-yl)-N-(2-phenoxyethyl)-1H-benzo[d]imidazole-1-carboxamide

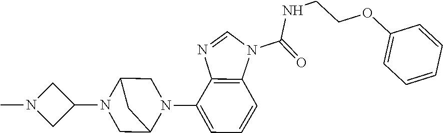 [SMILES: CN1CC(N2CC3CC2CN3c2cccc3c2ncn3C(=O)NCCOc2ccccc2)C1]

Following general procedure B, 4-(5-(1-methylazetidin-3-yl)-2,5-diazabicyclo[2.2.1]heptan-2-yl)-1H-benzo[d]imidazole (50 mg, 0.18 mmol) and (2-isocyanatoethoxy)benzene (15 mg, 0.45 mmol) afforded the title compound (7.4 mg, 9.4%) as a white solid. ¹H NMR (400 MHz, CDCl₃) δ 8.47 (bs, 1H), 7.33 (t, J=7.9 Hz, 2H), 7.22 (t, J=8.0 Hz, 1H), 7.11 (d, J=8.0 Hz, 1H), 7.12-6.84 (m, 3H), 6.78 (bs, 1H), 6.35 (d, J=8.0 Hz, 1H), 5.41 (s, 1H), 4.27-3.93 (m, 4H), 3.93 (dd, J=10.2, 5.2 Hz, 2H), 3.83-3.57 (m, 2H), 3.68-3.28 (m, 4H), 2.94 (d, J=9.1 Hz, 1H), 2.84 (d, J=9.4 Hz, 1H), 2.77 (s, 3H), 2.01-1.96 (m, 2H). LC-MS m/z: 447.1 [M+H]⁺. HPLC Purity (254 nm): 100%; t_{R}=5.40 min.

### Example 329—2-Methoxy-4-(3-(4-methylpiperazin-1-yl)azetidin-1-yl)-N-(2-phenoxyethyl)-1H-benzo[d]imidazole-1-carboxamide

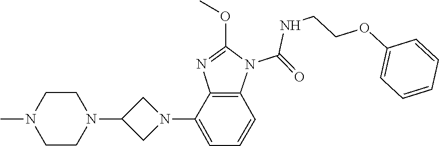 [SMILES: COc1nc2c(N3CC(N4CCN(C)CC4)C3)cccc2n1C(=O)NCCOc1ccccc1]

Following general procedure G (method A), 3-(3-(4-methylpiperazin-1-yl)azetidin-1-yl)benzene-1,2-diamine (2.0 g, 7.7 mmol) afforded 2-methoxy-4-(3-(4-methylpiperazin-1-yl)azetidin-1-yl)-1H-benzo[d]imidazole (800 mg, 34.8%) as a red oil. LC-MS m/z: 302.3 [M+H]⁺. Purity (254 nm): 88.11%; t_{R}=1.52 min.

Following general procedure B, 2-methoxy-4-(3-(4-methylpiperazin-1-yl)azetidin-1-yl)-1H-benzo[d]imidazole (100 mg, 0.33 mmol) and (2-isocyanatoethoxy)benzene (66 mg, 0.66 mmol) afforded the title compound (7.9 mg, 5.1%) as a white solid. ¹H NMR (400 MHz, CDCl₃) δ 7.61-7.50 (m, 2H), 7.34 (d, J=7.5 Hz, 2H), 7.09 (t, J=7.5 Hz, 1H), 7.04-6.93 (m, 3H), 6.31 (d, J=7.4 Hz, 1H), 4.35-4.18 (m, 4H), 4.25 (s, 3H), 3.97-3.84 (m, 4H), 3.40-3.35 (m, 1H), 2.50 (bs, 8H), 2.32 (s, 3H). LC-MS m/z: 465.0 [M+H]⁺. HPLC Purity (254 nm): 100%; t_{R}=6.87 min.

### Example 330—4-(3-((Dimethylamino)methyl)azetidin-1-yl)-N-(2-phenoxyethyl)-1H-benzo[d]imidazole-1-carboxamide

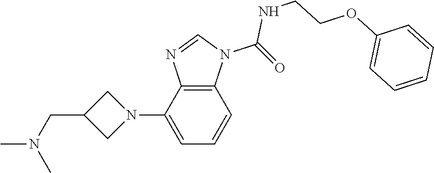 [SMILES: CN(C)CC1CN(c2cccc3c2ncn3C(=O)NCCOc2ccccc2)C1]

Following general procedure C, 1-(1-(1H-benzo[d]imidazol-4-yl)azetidin-3-yl)-N,N-dimethylmethanamine (115 mg, 0.5 mmol) and phenethoxybenzene (68 mg, 0.5 mmol) afforded the title compound (52.8 mg, 26.9%) as a white solid. ¹H NMR (400 MHz, CDCl₃) δ 8.42 (s, 1H), 8.31 (s, 1H), 7.31 (dd, J=15.2, 7.7 Hz, 2H), 7.21 (t, J=8.0 Hz, 1H), 7.07 (d, J=8.0 Hz, 1H), 7.00 (t, J=7.4 Hz, 1H), 6.94 (d, J=7.9 Hz, 2H), 6.45 (bs, 1H), 6.25 (d, J=7.8 Hz, 1H), 4.44 (t, J=7.9 Hz, 2H), 4.23 (t, J=5.0 Hz, 2H), 3.98-3.88 (m, 4H), 3.25-3.09 (m, 1H), 3.03 (d, J=7.0 Hz, 2H), 2.56 (s, 6H). LC-MS m/z: 394.3 [M+H]⁺. HPLC Purity (214 nm): 100%; t_{R}=6.64 min.

### Example 331—4-(3-((Dimethylamino)methyl)azetidin-1-yl)-N-(3-phenylprop-2-yn-1-yl)-1H-benzo[d]imidazole-1-carboxamide

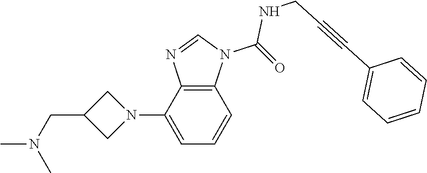 [SMILES: CN(C)CC1CN(c2cccc3c2ncn3C(=O)NCC#Cc2ccccc2)C1]

Following general procedure C, 1-(1-(1H-benzo[d]imidazol-4-yl)azetidin-3-yl)-N,N-dimethylmethanamine (80 mg, 0.35 mmol) and 3-phenylprop-2-yn-1-amine (46 mg, 0.35 mmol) afforded the title compound (9.3 mg, 6.9%) as a white solid. ¹H NMR (400 MHz, CDCl₃) δ 8.45 (s, 1H), 8.34 (s, 1H), 7.45 (dd, J=7.4, 2.2 Hz, 2H), 7.38-7.29 (m, 3H), 7.24-7.16 (m, 2H), 6.64 (bs, 1H), 6.24 (dd, J=7.2, 1.5 Hz, 1H), 4.52 (d, J=5.1 Hz, 2H), 4.42 (t, J=7.8 Hz, 2H), 3.94 (dd, J=7.9, 5.6 Hz, 2H), 3.23-3.09 (m, 1H), 3.06 (d, J=6.9 Hz, 2H), 2.56 (s, 6H). LC-MS m/z: 388.2 [M+H]⁺. HPLC Purity (214 nm): 100%; t_{R}=5.47 min.

### Example 332—4-(3-(4-Methylpiperazin-1-yl)azetidin-1-yl)-N-(3-phenylprop-2-yn-1-yl)-1H-benzo[d]imidazole-1-carboxamide

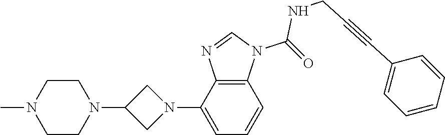 [SMILES: CN1CCN(C2CN(c3cccc4c3ncn4C(=O)NCC#Cc3ccccc3)C2)CC1]

Following general procedure C, 4-(3-(4-methylpiperazin-1-yl)azetidin-1-yl)-1H-benzo[d]imidazole (100 mg, 0.37 mmol) and 2-phenoxyethanamine (48 mg, 0.37 mmol) afforded the title compound (34.9 mg, 22.1%) as a white solid. ¹H NMR (400 MHz, CDCl₃) δ 8.32 (s, 1H), 7.51-7.46 (m, 2H), 7.38-7.33 (m, 3H), 7.23 (d, J=8.0 Hz, 1H), 7.09 (t, J=7.8 Hz, 1H), 6.29 (d, J=7.6 Hz, 1H), 6.07 (t, J=5.0 Hz, 1H), 4.56 (d, J=5.2 Hz, 2H), 4.39 (t, J=7.4 Hz, 2H), 4.10 (dd, J=7.9, 5.7 Hz, 2H), 3.39 (p, J=5.8 Hz, 1H), 2.52 (bs, 8H), 2.33 (s, 3H). LC-MS m/z: 429.2 [M+H]⁺. HPLC Purity (254 nm): 100%; t_{R}=6.27 min.

### Example 333—4-(4-(1-Methylazetidin-3-yl)piperazin-1-yl)-N-(3-phenylprop-2-yn-1-yl)-1H-benzo[d]imidazole-1-carboxamide

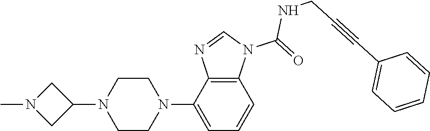 [SMILES: CN1CC(N2CCN(c3cccc4c3ncn4C(=O)NCC#Cc3ccccc3)CC2)C1]

Following general procedure C, 4-(3-(4-methylpiperazin-1-yl)azetidin-1-yl)-1H-benzo[d]imidazole (135 mg, 0.5 mmol) and 3-phenylprop-2-yn-1-amine (65 mg, 0.5 mmol) afforded the title compound (5.6 mg, 2.7%) as a white solid. ¹H NMR (400 MHz, CDCl₃) δ 8.37 (s, 1H), 7.48-7.39 (m, 3H), 7.38-7.28 (m, 4H), 6.75 (d, J=7.8 Hz, 1H), 6.23 (bs, 1H), 4.54 (d, J=5.1 Hz, 2H), 3.63 (d, J=5.7 Hz, 2H), 3.56 (bs, 4H), 3.12-3.07 (m, 3H), 2.65 (bs, 4H), 2.43 (s, 3H). LC-MS m/z: 429.1 [M+H]⁺. HPLC Purity (214 nm): 100%; t_{R}=6.07 min.

### Examples 334a and 334b—5-Fluoro-2-methoxy-6-morpholino-N-(2-phenethyl)-1H-benzo[d]imidazole-1-carboxamide and 6-fluoro-2-methoxy-5-morpholino-N-(2-phenethyl)-1H-benzo[d]imidazole-1-carboxamide

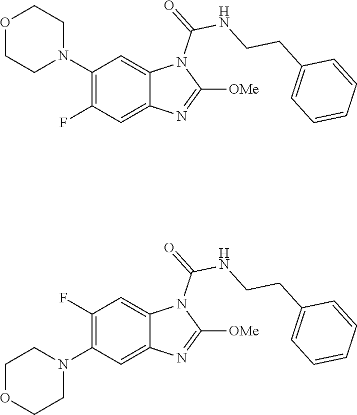 [SMILES: COc1nc2cc(F)c(N3CCOCC3)cc2n1C(=O)NCCc1ccccc1 ; COc1nc2cc(N3CCOCC3)c(F)cc2n1C(=O)NCCc1ccccc1]

Following general procedure C, 4-(6-fluoro-2-methoxy-1H-benzo[d]imidazol-5-yl)morpholine (150 mg, 0.60 mmol) and 2-phenylethan-1-amine (109 mg, 0.9 mmol) afforded 5-fluoro-2-methoxy-6-morpholino-N-(2-phenethyl)-1H-benzo[d]imidazole-1-carboxamide (20.7 mg, 8.7%) and 6-fluoro-2-methoxy-5-morpholino-N-(2-phenethyl)-1H-benzo[d]imidazole-1-carboxamide (31.4 mg, 13.2%) both as a white solids.

5-fluoro-2-methoxy-6-morpholino-N-(2-phenethyl)-1H-benzo[d]imidazole-1-carboxamide: ¹H NMR (400 MHz, CDCl₃) δ 7.86 (d, J=6.4 Hz, 1H), 7.38-7.35 (m, 2H), 7.30-7.25 (m, 3H), 7.17 (d, J=9.6 Hz, 1H), 6.95 (t, J=4.4 Hz, 1H), 4.11 (s, 3H), 3.89 (t, J=3.2 Hz, 4H), 3.72 (q, J=5.2 Hz, 2H), 3.09 (t, J=3.6 Hz, 4H), 2.96 (t, J=5.2 Hz, 2H). LC-MS m/z: 399.0 [M+H]⁺. HPLC Purity (214 nm): 100.00%; t_{R}=8.81 min.

6-fluoro-2-methoxy-5-morpholino-N-(2-phenethyl)-1H-benzo[d]imidazole-1-carboxamide: ¹H NMR (400 MHz, CDCl₃) δ 7.90 (d, J=10.4 Hz, 1H), 7.38-7.35 (m, 2H), 7.29-7.22 (m, 3H), 7.09 (d, J=6.4 Hz, 1H), 6.89 (t, J=4.0 Hz, 1H), 4.11 (s, 3H), 3.89 (t, J=3.6 Hz, 4H), 3.71 (q, J=5.2 Hz, 2H), 3.06 (t, J=3.2 Hz, 4H), 2.95 (t, J=6.4 Hz, 2H). LC-MS m/z: 399.0 [M+H]⁺. HPLC Purity (214 nm): 100.00%; t_{R}=8.86 min.

### Examples 335a and 335b—5-Fluoro-2-methoxy-6-morpholino-N-(2-phenoxyethyl)-1H-benzo[d]imidazole-1-carboxamide and 6-Fluoro-2-methoxy-5-morpholino-N-(2-phenoxy ethyl)-1H-benzo[d]imidazole-1-carboxamide

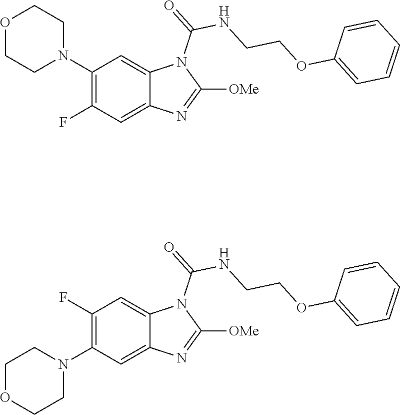 [SMILES: COc1nc2cc(F)c(N3CCOCC3)cc2n1C(=O)NCCOc1ccccc1 ; COc1nc2cc(N3CCOCC3)c(F)cc2n1C(=O)NCCOc1ccccc1]

Following general procedure C, 4-(6-fluoro-2-methoxy-1H-benzo[d]imidazol-5-yl)morpholine (120 mg, 0.48 mmol) and phenethoxybenzene (106 mg, 0.53 mmol) afforded 5-fluoro-2-methoxy-6-morpholino-N-(2-phenoxyethyl)-1H-benzo[d]imidazole-1-carboxamide (39.8 mg, 20.0%) and 6-fluoro-2-methoxy-5-morpholino-N-(2-phenoxyethyl)-1H-benzo[d]imidazole-1-carboxamide (27.2 mg, 13.8%) both as white solids.

5-fluoro-2-methoxy-6-morpholino-N-(2-phenoxyethyl)-1H-benzo[d]imidazole-1-carboxamide: ¹H NMR (400 MHz, CDCl₃) δ 7.85 (d, J=8.4 Hz, 1H), 7.46 (t, J=4.8 Hz, 1H), 7.33-7.29 (m, 2H), 7.21-7.18 (m, 1H), 7.00 (t, J=7.6 Hz, 1H), 6.93 (d, J=8.0 Hz, 2H), 4.28 (s, 3H), 4.19 (t, J=4.8 Hz, 2H), 3.89 (t, J=4.4 Hz, 4H), 3.85 (q, J=4.8 Hz, 2H), 3.09 (t, J=4.8 Hz, 4H). LC-MS m/z: 415.0 [M+H]⁺. HPLC Purity (214 nm): 100.00%; t_{R}=8.80 min.

6-fluoro-2-methoxy-5-morpholino-N-(2-phenoxyethyl)-1H-benzo[d]imidazole-1-carboxamide: ¹H NMR (400 MHz, CDCl₃) δ 7.90 (d, J=10.0 Hz, 1H), 7.43 (t, J=4.0 Hz, 1H), 7.33-7.29 (m, 2H), 7.11 (d, J=6.0 Hz, 1H), 7.00 (t, =6.0 Hz, 1H), 6.93 (d, J=6.4 Hz, 2H), 4.28 (s, 3H), 4.18 (t, J=4.0 Hz, 2H), 3.89 (t, J=2.8 Hz, 4H), 3.85 (q, J=4.0 Hz, 2H), 3.07 (t, J=3.2 Hz, 4H). LC-MS m/z: 415.0 [M+H]⁺. HPLC Purity (214 nm): 100.00%; t_{R}=8.84 min.

### Example 336—N-(3-(4-Fluorophenyl)prop-2-yn-1-yl)-4-(4-methylpiperazin-1-yl)-1H-benzo[d]imidazole-1-carboxamide

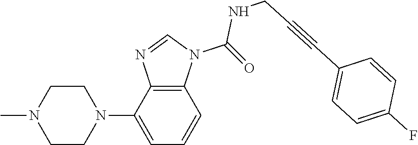 [SMILES: CN1CCN(c2cccc3c2ncn3C(=O)NCC#Cc2ccc(F)cc2)CC1]

Following general procedure C, 4-(4-methylpiperazin-1-yl)-1H-benzo[d]imidazole (0.15 g, 0.69 mmol) and 3-(4-fluorophenyl)prop-2-yn-1-amine (0.11 g, 0.76 mmol) afforded the title compound (50.5 mg, 18.6%) as a white solid. ¹H NMR (400 MHz, CDCl₃) δ 8.35 (s, 1H), 7.40 (dd, J=8.0, 0.4 Hz, 1H), 7.33-7.21 (m, 3H), 7.16-7.12 (m, 1H), 7.09-7.05 (m, 1H), 6.76 (d, J=6.0 Hz, 1H), 5.99 (bs, 1H), 4.53 (d, J=4.0 Hz, 2H), 3.57 (bs, 4H), 2.70 (bs, 4H), 2.39 (s, 3H). LC-MS m/z: 392.2 [M+H]⁺. HPLC Purity (214 nm): 100%; t_{R}=5.53 min.

### Example 337—N-(3-(3-Fluorophenyl)prop-2-yn-1-yl)-4-(4-methylpiperazin-1-yl)-1H-benzo[d]imidazole-1-carboxamide

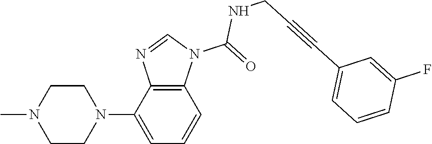 [SMILES: CN1CCN(c2cccc3c2ncn3C(=O)NCC#Cc2cccc(F)c2)CC1]

Following general procedure C, 4-(4-methylpiperazin-1-yl)-1H-benzo[d]imidazole (0.20 g, 0.92 mmol) and 3-(3-fluorophenyl)prop-2-yn-1-amine (0.15 g, 1.02 mmol) afforded the title compound (134.6 mg, 37.0%) as a white solid. ¹H NMR (400 MHz, CDCl₃): b 8.37 (s, 1H), 740-6.75 (m, 6H), 6.76 (d, J=7.6 Hz, 1H), 6.12 (brs, 1H), 4.54 (d, J=5.2 Hz, 2H), 3.58 (brs, 4H), 2.74 (t, J=4.8 Hz, 4H), 2.42 (s, 3H). LC-MS m/z: 392.2 [M+H]⁺. HPLC Purity (214 nm): 98%; t_{R}=5.60 min.

### Example 338—N-(3-(2-Fluorophenyl)prop-2-yn-1-yl)-4-(4-methylpiperazin-1-yl)-1H-benzo[d]imidazole-1-carboxamide

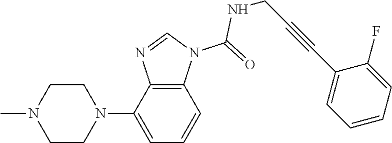 [SMILES: CN1CCN(c2cccc3c2ncn3C(=O)NCC#Cc2ccccc2F)CC1]

Following general procedure C, 4-(4-methylpiperazin-1-yl)-1H-benzo[d]imidazole (150 mg, 0.69 mmol) and 3-(2-fluorophenyl)prop-2-yn-1-amine (255 mg, 1.38 mmol) afforded the title compound (13.8 mg, 5.1%) as a white solid. ¹H NMR (500 MHz, CDCl₃) δ 8.48 (s, 1H), 7.54 (d, J=8.0 Hz, 1H), 7.45 (td, J=3.5 Hz, 1.5 Hz, 1H), 7.33-7.28 (m, 2H), 7.12-7.06 (m, 2H), 6.95 (s, 1H), 6.74 (d, J=7.5 Hz, 1H), 4.57 (d, J=5.0 Hz, 2H), 3.68 (bs, 4H), 3.11 (bs, 4H), 2.62 (s, 3H). LC-MS m/z: 392.1 [M+H]⁺. HPLC Purity (254 nm): 97.31%; t_{R}=6.04 min.

### Example 339—N-(3-(4-Methoxyphenyl)prop-2-yn-1-yl)-4-(4-methylpiperazin-1-yl)-1H-benzo[d]imidazole-1-carboxamide

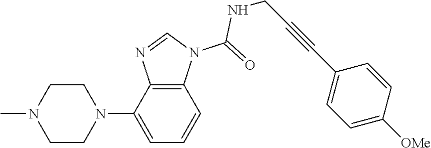 [SMILES: COc1ccc(C#CCNC(=O)n2cnc3c(N4CCN(C)CC4)cccc32)cc1]

Following general procedure C, 4-(4-methylpiperazin-1-yl)-1H-benzo[d]imidazole (145 mg, 0.67 mmol) and 3-(4-methoxyphenyl)prop-2-yn-1-amine (148 mg, 1.74 mmol) afforded the title compound (22.3 mg, 12.0%) as a white solid. ¹H NMR (500 MHz, CDCl₃) δ 8.35 (s, 1H), 7.40-7.36 (m, 3H), 7.30 (t, J=8.0 Hz, 1H), 6.86-6.84 (m, 2H), 6.76 (d, J=7.5 Hz, 1H), 5.99 (t, J=4.5 Hz, 1H), 4.52 (d, J=5.5 Hz, 2H), 3.81 (s, 3H), 3.57 (bs, 4H), 2.70 (t, J=4.5 Hz, 4H), 2.39 (s, 3H). LC-MS m/z: 404.1 [M+H]⁺. HPLC Purity (214 nm): 100.00%; t_{R}=6.05 min.

### Example 340—N-(3-(3-Methoxyphenyl)prop-2-yn-1-yl)-4-(4-methylpiperazin-1-yl)-1H-benzo[d]imidazole-1-carboxamide

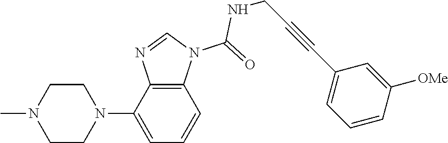 [SMILES: COc1cccc(C#CCNC(=O)n2cnc3c(N4CCN(C)CC4)cccc32)c1]

Following general procedure C, 4-(4-methylpiperazin-1-yl)-1H-benzo[d]imidazole (100 mg, 0.46 mmol) and 3-(3-methoxyphenyl)prop-2-yn-1-amine (80 mg, 0.5 mmol) afforded the title compound (5.0 mg, 2.7%) as a white solid. ¹H NMR (400 MHz, CDCl₃) δ 8.37 (s, 1H), 7.40 (d, J=7.7 Hz, 1H), 7.29 (dd, J=14.0, 5.9 Hz, 1H), 7.23 (dd, J=10.8, 5.1 Hz, 1H), 7.04 (d, J=7.6 Hz, 1H), 6.96 (dd, J=9.9, 8.6 Hz, 1H), 6.93-6.87 (m, 1H), 6.74 (dd, J=11.8, 5.5 Hz, 1H), 6.18 (t, J=5.0 Hz, 1H), 4.53 (d, J=5.2 Hz, 2H), 3.83 (s, 3H), 3.59 (bs, 4H), 2.78-2.64 (m, 4H), 2.38 (s, 3H). LC-MS m/z: 404.1 [M+H]⁺. HPLC Purity (214 nm): 98.37%; t_{R}=5.38 min.

### Example 341—N-(3-(2-Methoxyphenyl)prop-2-yn-1-yl)-4-(4-methylpiperazin-1-yl)-1H-benzo[d]imidazole-1-carboxamide

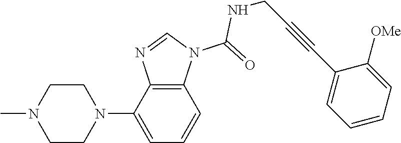 [SMILES: COc1ccccc1C#CCNC(=O)n1cnc2c(N3CCN(C)CC3)cccc21]

Following general procedure C, 4-(4-methylpiperazin-1-yl)-1H-benzo[d]imidazole (108 mg, 0.5 mmol) and 3-(2-methoxyphenyl)prop-2-yn-1-amine (81.0 mg, 0.5 mmol) afforded the title compound (60.2 mg, 25%) as a white solid. ¹H NMR (500 MHz, CDCl₃) δ 8.48 (d, J=9.5 Hz, 2H), 7.58 (d, J=7.7 Hz, 1H), 7.42 (d, J=6.7 Hz, 1H), 7.37-7.27 (m, 2H), 6.95-6.90 (m, 2H), 6.73 (d, J=7.8 Hz, 1H), 4.58 (d, J=4.5 Hz, 2H), 3.88 (s, 3H), 3.69 (bs, 4H), 3.16 (bs, 4H), 2.65 (s, 3H). LC-MS m/z: 404.1 [M+H]⁺. HPLC Purity (214 nm): 99.34%; t_{R}=5.25 min.

### Example 342—N-(4-Ethynylbenzyl)-4-(4-methylpiperazin-1-yl)-1H-benzo[d]imidazole-1-carboxamide

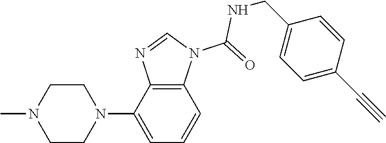 [SMILES: C#Cc1ccc(CNC(=O)n2cnc3c(N4CCN(C)CC4)cccc32)cc1]

Following general procedure C, 4-(4-methylpiperazin-1-yl)-1H-benzo[d]imidazole (216 mg, 0.6 mmol) and (4-ethynylphenyl)methanamine (99.0 mg, 0.6 mmol) afforded the title compound (43.7 mg, 25%) as a white solid. ¹H NMR (500 MHz, CDCl₃) δ 8.36 (s, 1H), 7.50 (d, J=8.0 Hz, 2H), 7.36 (d, J=8.0 Hz, 2H), 7.28-7.26 (m, 2H), 6.74 (dd, J=6.4, 2.3 Hz, 1H), 6.17 (bs, 1H), 4.69 (d, J=5.7 Hz, 2H), 3.58 (bs, 4H), 3.10 (s, 1H), 2.70 (t, J=4.7 Hz, 4H), 2.39 (s, 3H). LC-MS m/z: 374.0 [M+H]⁺. HPLC Purity (214 nm): 100%; t_{R}=7.28 min.

### Example 343—N-(2-Methyl-2-phenoxypropyl)-4-(3-(4-methylpiperazin-1-yl)azetidin-1-yl)-1H-benzo[d]imidazole-1-carboxamide

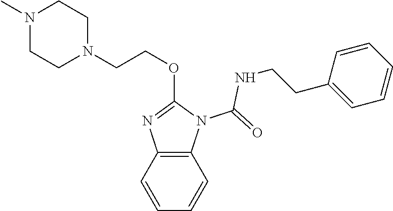 [SMILES: CN1CCN(CCOc2nc3ccccc3n2C(=O)NCCc2ccccc2)CC1]

Following general procedure C, 2-(2-(4-methylpiperazin-1-yl)ethoxy)-1H-benzo[d]imidazole (130 mg, 0.5 mmol) and 2-phenylethan-1-amine (60 mg, 0.5 mmol) afforded the title compound (8.4 mg, 3.9%) as a white solid. ¹H NMR (400 MHz, CDCl₃) δ 8.18 (t, J=6.0 Hz, 1H), 7.52-7.48 (m, 2H), 7.37-7.32 (m, 2H), 7.25-7.14 (m, 4H), 4.64 (t, J=5.5 Hz, 2H), 3.71 (dd, J=13.0, 6.8 Hz, 2H), 2.97 (t, J=6.9 Hz, 2H), 2.64 (t, J=5.5 Hz, 2H), 2.37 (bs, 4H), 2.27 (s, 3H), 1.66 (bs, 4H). LC-MS m/z: 408.3 [M+H]⁺. HPLC Purity (214 nm): 95.06%; t_{R}=5.35 min.

### Example 344—N-(2-Methyl-2-phenoxypropyl)-4-(3-(4-methylpiperazin-1-yl)azetidin-1-yl)-1H-benzo[d]imidazole-1-carboxamide

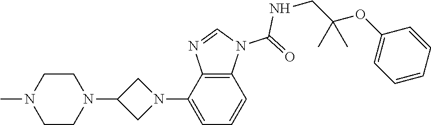 [SMILES: CN1CCN(C2CN(c3cccc4c3ncn4C(=O)NCC(C)(C)Oc3ccccc3)C2)CC1]

To a solution of 2-methyl-2-phenoxypropanoic acid (3.00 g, 16.65 mmol) in DCM (30.0 mL) was added DMF (8 drops) and oxalyl chloride (3.17 g, 24.97 mmol) at 0° C. and the reaction mixture was stirred at RT for 1 h. The mixture was concentrated and the residue was redissolved in THF (10.0 mL) to which was added NH₃/H₂O (20.0 mL) at 0° C. After addition, the mixture was stirred at RT for 30 min and additional H₂O was added (20.0 mL). The mixture was extracted with DCM (100.0 mL×2) and the organic layers were dried over anhydrous Na₂SO₄, filtered and concentrated to give 2-methyl-2-phenoxypropanamide (2.1 g, 70.0%) as a white solid. LC-MS m/z: 180.2 [M+H]⁺. Purity (214 nm): 93%; t_{R}=1.60 min.

A solution of 2-methyl-2-phenoxypropanamide (2.10 g, 11.72 mmol) in BH₃/THF (30.0 mL) was stirred at 70° C. for 14 h. The reaction mixture was cooled, quenched with water (20.0 mL), extracted with DCM (50.0 mL×2), dried over anhydrous Na₂SO₄, filtered and concentrated to give 2-methyl-2-phenoxypropan-1-amine (1.2 g, 63.1%) as a yellow oil. LC-MS m/z: 166.3 [M+H]⁺. Purity (214 nm): 65%; t_{R}=1.57 min.

Following general procedure C, 4-(3-(4-methylpiperazin-1-yl)azetidin-1-yl)-1H-benzo[d]imidazole (0.15 g, 0.55 mmol) and 2-methyl-2-phenoxypropan-1-amine (0.14 g, 0.83 mmol) afforded the title compound (61.3 mg, 24%) as a white solid. ¹H NMR (400 MHz, DMSO-d₆) δ 8.65 (bs, 1H), 8.26 (s, 1H), 7.31 (q, J=8.4 Hz, 3H), 7.14-7.05 (m, 4H), 6.20 (d, J=7.2 Hz, 1H), 4.21 (t, J=7.2 Hz, 2H), 3.87-3.82 (m, 2H), 3.55 (d, J=6.4 Hz, 2H), 3.27-3.21 (m, 1H), 2.35 (bs, 8H), 2.15 (s, 3H), 1.27 (s, 6H). LC-MS: m/z: 463.2 [M+H]⁺. HPLC Purity (214 nm): 97%; t_{R}=6.24 min.

### Example 345—N-(Benzo[d][1,3]dioxol-2-ylmethyl)-4-(4-methylpiperazin-1-yl)-1H-benzo[d]imidazole-1-carboxamide

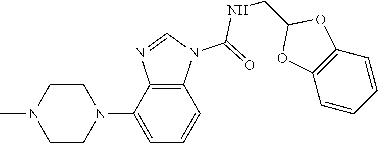 [SMILES: CN1CCN(c2cccc3c2ncn3C(=O)NCC2Oc3ccccc3O2)CC1]

Following general procedure C, 4-(4-methylpiperazin-1-yl)-1H-benzo[d]imidazole (0.15 g, 0.69 mmol) and benzo[d][1,3]dioxol-2-ylmethanamine (0.12 g, 0.76 mmol) afforded the title compound (69.1 mg, 25.3%) as a white solid. ¹H NMR (400 MHz, DMSO-d₆) δ 8.94 (bs, 1H), 8.17 (s, 1H), 7.47 (d, J=6.0 Hz, 1H), 7.16 (t, J=6.0 Hz, 1H), 6.91 (dd, J=4.4, 2.4 Hz, 2H) 6.83 (dd, J=4.4, 2.4 Hz, 4H), 6.65 (d, J=6.0 Hz, 1H), 6.45 (t, J=4.0 Hz, 1H), 3.78 (dd, J=4.8, 3.2 Hz, 2H), 3.45 (bs, 4H), 3.28 (bs, 4H), 3.24 (s, 3H). LC-MS m/z: 394.3 [M+H]⁺. HPLC Purity (214 nm): 100%; t_{R}=5.20 min.

### Example 346—N-(Benzo[d][1,3]dioxol-5-ylmethyl)-4-(4-methylpiperazin-1-yl)-1H-benzo[d]imidazole-1-carboxamide

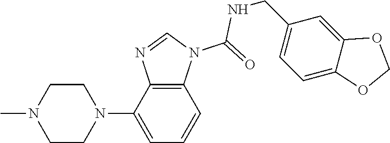 [SMILES: CN1CCN(c2cccc3c2ncn3C(=O)NCc2ccc3c(c2)OCO3)CC1]

Following general procedure C, 4-(4-methylpiperazin-1-yl)-1H-benzo[d]imidazole (0.15 g, 0.69 mmol) and benzo[d][1,3]dioxol-5-ylmethanamine (0.12 g, 0.76 mmol) afforded the title compound (142.6 mg, 39.2%) as a white solid. ¹H NMR (400 MHz, CDCl₃) δ 8.46 (s, 1H), 7.44 (d, J=6.4 Hz, 1H), 7.26-7.24 (m, 1H), 6.91 (d, J=1.2 Hz, 1H), 6.86 (dd, J=6.4, 1.2 Hz, 1H), 6.79 (d, J=6.4 Hz, 1H), 6.79-6.75 (m, 1H), 6.70 (d, J=6.4 Hz, 1H), 5.96 (s, 2H), 4.57 (d, J=4.8 Hz, 2H), 3.67 (bs, 4H), 3.10 (bs, 4H), 2.61 (s, 3H). LC-MS m/z: 394.2 [M+H]⁺. HPLC Purity (214 nm): 98%; t_{R}=5.13 min.

### Example 347—N-(Indolizin-7-ylmethyl)-4-(4-methylpiperazin-1-yl)-1H-benzo[d]imidazole-1-carboxamide

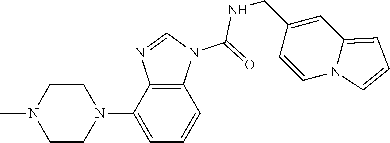 [SMILES: CN1CCN(c2cccc3c2ncn3C(=O)NCc2ccn3cccc3c2)CC1]

Following general procedure C, 4-(4-methylpiperazin-1-yl)-1H-benzo[d]imidazole (108 mg, 0.5 mmol) and indolizin-7-ylmethanamine (51.0 mg, 0.5 mmol) afforded the title compound (44.9 mg, 31%) as a white solid. ¹H NMR (500 MHz, CDCl₃) δ 8.38 (s, 1H), 7.95 (d, J=7.1 Hz, 1H), 7.35 (s, 1H), 7.31 (s, 1H), 7.30-7.27 (m, 2H), 6.80 (dd, J=3.8, 2.7 Hz, 1H), 6.74 (dd, J=7.0, 1.9 Hz, 1H), 6.51 (dd, J=7.2, 1.8 Hz, 1H), 6.44 (d, J=3.9 Hz, 1H), 6.06 (s, 1H), 4.60 (d, J=5.6 Hz, 2H), 3.69-3.43 (m, 4H), 2.75-2.69 (m, 4H), 2.41 (s, 3H). LC-MS m/z: 389.3 [M+H]⁺. HPLC Purity (214 nm): 100%; t_{R}=5.18 min.

### Example 348—2-(2-(4-Methylpiperazin-1-yl)ethoxy)-N-(3-phenylprop-2-yn-1-yl)-1H-benzo[d]imidazole-1-carboxamide

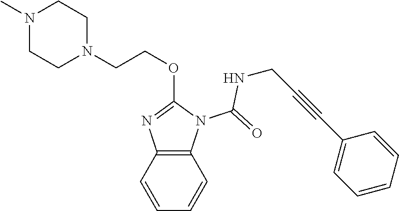 [SMILES: CN1CCN(CCOc2nc3ccccc3n2C(=O)NCC#Cc2ccccc2)CC1]

Following general procedure C, 2-(2-(4-methylpiperazin-1-yl)ethoxy)-1H-benzo[d]imidazole (73 mg, 0.28 mmol) and 3-phenylprop-2-yn-1-amine (40 mg, 0.3 mmol) afforded the title compound (5.2 mg, 4.4%) as a white solid. ¹H NMR (400 MHz, CDCl₃) δ 8.22-8.13 (m, 1H), 7.88 (t, J=5.1 Hz, 1H), 7.49 (dd, J=5.9, 3.1 Hz, 1H), 7.46-7.37 (m, 2H), 7.37-7.28 (m, 3H), 7.26-7.20 (m, 2H), 4.76 (t, J=5.4 Hz, 2H), 4.49 (d, J=5.4 Hz, 2H), 2.86 (dd, J=10.4, 5.1 Hz, 2H), 2.74-2.31 (m, 8H), 2.22 (s, 3H). LC-MS m/z: 418.1 [M+H]⁺. HPLC Purity (214 nm): 100%; t_{R}=5.49 min.

### Example 349—4-(3-(4-Methylpiperazin-1-yl)azetidin-1-yl)-N-phenethyl-1H-benzo[d]imidazole-1-carboxamide

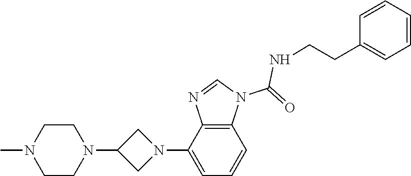 [SMILES: CN1CCN(C2CN(c3cccc4c3ncn4C(=O)NCCc3ccccc3)C2)CC1]

Following general procedure B, 4-(3-(4-methylpiperazin-1-yl)azetidin-1-yl)-1H-benzo[d]imidazole (100 mg, 0.37 mmol) and (2-isocyanatoethyl)benzene (60.3 mg, 0.41 mmol) afforded the title compound (76.3 mg, 49.5%) as a white solid. ¹H NMR (400 MHz, CDCl₃) δ 8.25 (s, 1H), 7.37 (d, J=7.3 Hz, 2H), 7.36-7.29 (m, 3H), 7.13 (t, J=8.0 Hz, 1H), 6.78 (d, J=8.0 Hz, 1H), 6.24 (d, J=7.9 Hz, 1H), 6.01 (s, 1H), 4.36 (t, J=7.4 Hz, 2H), 4.05 (dd, J=7.8, 5.5 Hz, 2H), 3.80 (dd, J=12.6, 6.7 Hz, 4H), 3.43 (t, J=5.9 Hz, 1H), 3.03 (t, J=6.8 Hz, 2H), 2.90 (s, 3H), 2.67 (s, 3H), 2.55 (d, J=6.6 Hz, 3H). LC-MS m/z: 419.1 [M+H]⁺. HPLC Purity (254 nm): 100%; t_{R}=5.92 min.

### Example 350—4-(7-Methyl-2,7-diazaspiro[3.5]nonan-2-yl)-N-(2-phenoxyethyl)-1H-benzo[d]imidazole-1-carboxamide

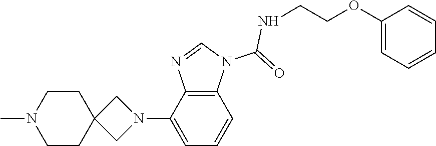 [SMILES: CN1CCC2(CC1)CN(c1cccc3c1ncn3C(=O)NCCOc1ccccc1)C2]

Following general procedure E, 3-fluoro-2-nitroaniline (900 mg, 5.8 mmol) and 7-methyl-2,7-diazaspiro[3.5]nonane (1.2 g, 6.93 mmol) afforded 3-(7-methyl-2,7-diazaspiro[3.5]nonan-2-yl)-2-nitroaniline (700 mg, 44.1%) as a red solid. LC-MS m/z: 277.2 [M+H]⁺. Purity (214 nm): 97.48%; t_{R}=1.65 min.

Following general procedure F, 3-(7-methyl-2,7-diazaspiro[3.5]nonan-2-yl)-2-nitroaniline (700 mg, 2.54 mmol) afforded 3-(7-methyl-2,7-diazaspiro[3.5]nonan-2-yl)benzene-1,2-diamine (400 mg, 99.0%) as a yellow oil. LC-MS m/z: 247.2 [M+H]⁺. Purity (254 nm): 99.45%; t_{R}=1.36 min.

Following general procedure G (method B), 3-(7-methyl-2,7-diazaspiro[3.5]nonan-2-yl)benzene-1,2-diamine (400 mg, 1.63 mmol) afforded 2-(1H-benzo[d]imidazol-4-yl)-7-methyl-2,7-diazaspiro[3.5]nonane (210 mg, 51.1%) as a brown solid. LC-MS m/z: 257.2 [M+H]⁺. Purity (254 nm): 100.00%; t_{R}=1.49 min.

Following general procedure C, 2-(1H-benzo[d]imidazol-4-yl)-7-methyl-2,7-diazaspiro[3.5]nonane (60 mg, 0.23 mmol) and 2-phenoxyethan-1-amine (37 mg, 0.28 mmol) afforded the title compound (14.0 mg, 14.3%) as a white solid. ¹H NMR (400 MHz, CDCl₃) δ 8.32 (s, 1H), 7.33-7.29 (m, 2H), 7.21 (t, J=8.0 Hz, 1H), 7.07 (d, J=8.0 Hz, 1H), 7.00 (t, J=7.6 Hz, 1H), 6.94 (d, J=8.0 Hz, 2H), 6.55 (t, J=5.2 Hz, 1H), 6.24 (d, J=7.6 Hz, 1H), 4.23 (t, J=4.8 Hz, 2H), 4.0 (s, 4H), 3.92 (q, J=4.8 Hz, 2H), 2.84 (s, 4H), 2.52 (s, 3H), 2.11 (t, J=5.2 Hz, 4H). LC-MS m/z: 420.3 [M+H]⁺. HPLC Purity (214 nm): 97.87%; t_{R}=8.80 min.

### Example 351—4-(2-Methyl-2,7-diazaspiro[3.5]nonan-7-yl)-N-(2-phenoxyethyl)-1H-benzo[d]imidazole-1-carboxamide

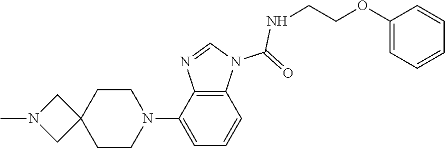 [SMILES: CN1CC2(CCN(c3cccc4c3ncn4C(=O)NCCOc3ccccc3)CC2)C1]

Following general procedure E, 3-fluoro-2-nitroaniline (600 mg, 3.84 mmol) and 2-methyl-2,7-diazaspiro[3.5]nonane (1.35 g, 7.69 mmol) afforded 3-(2-methyl-2,7-diazaspiro[3.5]nonan-7-yl)-2-nitroaniline (1.0 g, 94.3%) as a red solid. LC-MS m/z: 277.2 [M+H]⁺. Purity (214 nm): 98.17%; t_{R}=1.57 min.

Following general procedure F, 3-(2-methyl-2,7-diazaspiro[3.5]nonan-7-yl)-2-nitroaniline (1.0 g, 3.60 mmol) afforded 3-(2-methyl-2,7-diazaspiro[3.5]nonan-7-yl)benzene-1,2-diamine (800 mg, 90.4%) as a red solid. LC-MS m/z: 247.2 [M+H]⁺. Purity (214 nm): 99.42%; t_{R}=1.35 min.

Following general procedure G (method A), 3-(2-methyl-2,7-diazaspiro[3.5]nonan-7-yl)benzene-1,2-diamine (800 mg, 3.25 mmol) afforded 7-(1H-benzo[d]imidazol-4-yl)-2-methyl-2,7-diazaspiro[3.5]nonane (800 mg, 96.0%) as a brown solid. LC-MS m/z: 257.2 [M+H]⁺. Purity (254 nm): 89.94%; t_{R}=1.40 min.

Following general procedure C, 7-(1H-benzo[d]imidazol-4-yl)-2-methyl-2,7-diazaspiro[3.5]nonane (150 mg, 0.59 mmol) and 2-phenoxyethan-1-amine (97 mg, 0.71 mmol) afforded the title compound (7.8 mg, 4.8%) as a yellow solid. ¹H NMR (500 MHz, CDCl₃) δ 8.41 (s, 1H), 7.40 (d, J=8.5 Hz, 1H), 7.32-7.23 (m, 3H), 6.99 (t, J=7.5 Hz, 1H), 6.94-6.90 (m, 3H), 6.69 (d, J=8.0 Hz, 1H), 4.23 (t, J=5.0 Hz, 2H), 3.91 (q, J=5.0 Hz, 2H), 3.70 (s, 4H), 3.39 (t, J=4.5 Hz, 4H), 2.73 (s, 3H), 2.06 (t, J=5.0 Hz, 4H). LC-MS m/z: 420.0 [M+H]⁺. HPLC Purity (214 nm): 99.18%; t_{R}=5.41 min.

### Example 352—4-(4-Methylpiperazin-1-yl)-N-(4-phenylbut-3-yn-1-yl)-1H-benzo[d]imidazole-1-carboxamide

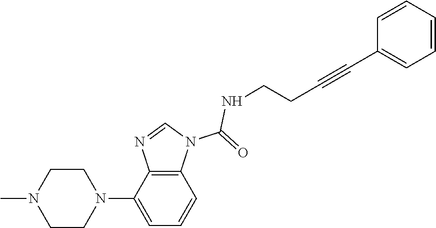 [SMILES: CN1CCN(c2cccc3c2ncn3C(=O)NCCC#Cc2ccccc2)CC1]

Following general procedure C, 4-(4-methylpiperazin-1-yl)-1H-benzo[d]imidazole (300 mg, 1.4 mmol) and 4-phenylbut-3-yn-1-amine (218 mg, 1.5 mmol) afforded the title compound (77.1 mg, 14.3%) as a white solid. ¹H NMR (400 MHz, CDCl₃) δ 8.50-8.43 (m, 1H), 7.51 (d, J=8.3 Hz, 1H), 7.43 (dd, J=7.0, 2.4 Hz, 2H), 7.43-7.30 (m, 3H), 7.18 (t, J=8.1 Hz, 1H), 6.74 (d, J=8.0 Hz, 1H), 3.78 (dd, J=12.1, 6.1 Hz, 2H), 3.77-3.45 (m, 4H), 3.03 (t, J=16.7 Hz, 4H), 2.87 (t, J=6.3 Hz, 2H), 2.60 (s, 3H). LC-MS m/z: 388.3 [M+H]⁺. HPLC Purity (254 nm): 97.67%; t_{R}=5.58 min.

### Example 353—4-(3-(Dimethylamino)azetidin-1-yl)-N-(2-phenoxyethyl)-1H-benzo[d]imidazole-1-carboxamide

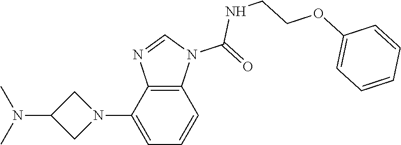 [SMILES: CN(C)C1CN(c2cccc3c2ncn3C(=O)NCCOc2ccccc2)C1]

Following general procedure C, 1-(1H-benzo[d]imidazol-4-yl)-N,N-dimethylazetidin-3-amine (0.10 g, 0.46 mmol) and 2-phenoxyethanamine (0.10 g, 0.69 mmol) afforded the title compound (50.1 mg, 15.9%) as a white solid. ¹H NMR (400 MHz, DMSO-d₆) δ 8.73 (bs, 1H), 8.50 (s, 1H), 7.36 (d, J=6.4 Hz, 1H), 7.30 (t, J=6.0 Hz, 2H), 7.12 (t, J=6.8 Hz, 1H), 6.99-6.92 (m, 3H), 6.20 (d, J=6.4 Hz, 1H), 4.22-4.16 (m, 4H), 3.83-3.80 (m, 2H), 3.67 (q, J=4.4 Hz, 2H), 3.17 (m, 1H), 2.11 (s, 6H). LC-MS: m/z: 380.0 [M+H]⁺. HPLC Purity (214 nm): 100%; t_{R}=5.16 min.

### Example 354—4-(3-(Methylamino)azetidin-1-yl)-N-(2-phenoxyethyl)-1H-benzo[d]imidazole-1-carboxamide

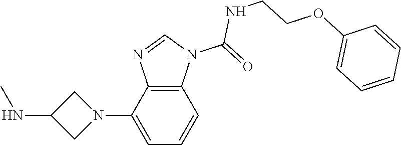 [SMILES: CNC1CN(c2cccc3c2ncn3C(=O)NCCOc2ccccc2)C1]

Following general procedure E, N-methylazetidin-3-amine (2.9 g, 33.7 mmol) and 3-fluoro-2-nitroaniline (5.2 g, 33.7 mmol) afforded 1-(3-amino-2-nitrophenyl)-N-methylazetidin-3-amine (1.8 g, 24.0%) as a red solid. LC-MS m/z: 223.0 [M+H]⁺. Purity (254 nm): 100.0%; t_{R}=1.31 min.

Following general procedure F, 1-(3-amino-2-nitrophenyl)-N-methylazetidin-3-amine (1.8 g, 8.1 mmol) in MeOH (15 mL) afforded 3-(3-(methylamino)azetidin-1-yl)benzene-1,2-diamine (1.4 g, 87.5%) as a red solid. LC-MS m/z: 193.2 [M+H]⁺. Purity (254 nm): 84.54%; t_{R}=0.99 min.

Following general procedure G (method B), 3-(3-(methylamino)azetidin-1-yl)benzene-1,2-diamine (1.4 g, 7.3 mmol) afforded 1-(1H-benzo[d]imidazol-4-yl)-N-methylazetidin-3-amine (120 mg, 8.1%) as a white solid. LC-MS m/z: 203.2 [M+H]⁺. Purity (254 nm): 90.98%; t_{R}=1.22 min.

Following general procedure C, 1-(1H-benzo[d]imidazol-4-yl)-N-methylazetidin-3-amine (120 mg, 0.59 mmol) and 2-phenoxyethanamine (80 mg, 0.59 mmol) afforded the title compound (1.3 mg, 0.6%) as a white solid. ¹H NMR (400 MHz, DMSO) δ 7.99 (s, 1H), 7.33-7.26 (m, 2H), 7.02-6.90 (m, 4H), 6.84 (d, J=7.3 Hz, 1H), 6.69 (s, 1H), 6.06 (d, J=7.8 Hz, 1H), 5.03 (d, J=5.9 Hz, 1H), 4.33 (t, J=8.0 Hz, 2H), 4.00 (dd, J=13.8, 7.5 Hz, 4H), 3.40 (d, J=5.9 Hz, 3H), 2.90 (s, 3H). LC-MS m/z: 366.1 [M+H]⁺. HPLC Purity (254 nm): 100%; t_{R}=7.17 min.

### Example 355—4-(3-(Dimethylamino)azetidin-1-yl)-N-(4-methylpent-2-yn-1-yl)-1H-benzo[d]imidazole-1-carboxamide

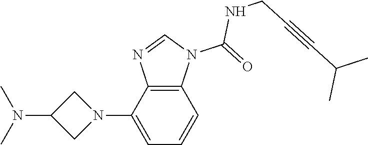 [SMILES: CC(C)C#CCNC(=O)n1cnc2c(N3CC(N(C)C)C3)cccc21]

Following general procedure C, 1-(1H-benzo[d]imidazol-4-yl)-N,N-dimethylazetidin-3-amine (0.10 g, 0.46 mmol) and 4-methylpent-2-yn-1-amine (0.07 g, 0.69 mmol) afforded the title compound (21.1 mg, 13.5%) as a white solid. ¹H NMR (400 MHz, CDCl₃) δ 8.28 (s, 1H), 7.22 (t, J=8.0 Hz, 1H), 7.07 (d, J=8.0 Hz, 2H), 6.28 (d, J=7.6 Hz, 1H), 5.92 (br, 1H), 4.37 (t, J=8.0 Hz, 2H), 4.27 (q, J=2.0 Hz, 2H), 4.09 (q, J=5.6 Hz, 2H), 3.40 (bs, 1H), 2.58 (bs, 1H), 2.31 (s, 6H), 1.87 (d, J=6.8 Hz, 6H). LC-MS: m/z: 340.1 [M+H]⁺. HPLC Purity (214 nm): 96%; t_{R}=5.22 min.

### Example 356—4-(3-(5-Methyl-2,5-diazabicyclo[2.2.1]heptan-2-yl)azetidin-1-yl)-N-(2-phenoxyethyl)-1H-benzo[d]imidazole-1-carboxamide

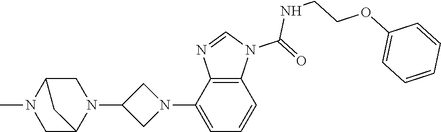 [SMILES: CN1CC2CC1CN2C1CN(c2cccc3c2ncn3C(=O)NCCOc2ccccc2)C1]

Following general procedure E, 2-(azetidin-3-yl)-5-methyl-2,5-diazabicyclo[2.2.1]heptanes (1.6 g, 1.9 mmol) and 3-fluoro-2-nitroaniline (300 mg, 1.9 mmol) afforded 3-(3-(5-methyl-2,5-diazabicyclo[2.2.1]heptan-2-yl)azetidin-1-yl)-2-nitroaniline (450 mg, 41%) as a red solid. LC-MS m/z: 304.2 [M+H]⁺. Purity (254 nm): 81.40%; t_{R}=1.47 min.

Following general procedure F, 3-(3-(5-methyl-2,5-diazabicyclo[2.2.1]heptan-2-yl)azetidin-1-yl)-2-nitroaniline (450 mg, 1.5 mmol) afforded 3-(3-(5-methyl-2,5-diazabicyclo[2.2.1]heptan-2-yl)azetidin-1-yl)benzene-1,2-diamine (412 mg, 90.9%) as a red oil. LC-MS m/z: 274.2 [M+H]⁺. Purity (214 nm): 100%; t_{R}=1.02 min.

Following general procedure G (method B), 3-(3-(5-methyl-2,5-diazabicyclo[2.2.1]heptan-2-yl)azetidin-1-yl)benzene-1,2-diamine (390 mg, 1.43 mmol) afforded 4-(3-(5-methyl-2,5-diazabicyclo[2.2.1]heptan-2-yl)azetidin-1-yl)-1H-benzo[d]imidazole (295 mg, 100%) as a red solid. LC-MS m/z: 284.3 [M+H]⁺. Purity (214 nm): 85.89%; t_{R}=1.38 min.

Following general procedure C, 4-(3-(5-methyl-2,5-diazabicyclo[2.2.1]heptan-2-yl)azetidin-1-yl)-1H-benzo[d]imidazole (110 mg, 0.39 mmol) and 2-phenoxyethan-1-amine (53 mg, 0.39 mmol) afforded the title compound (2.9 mg, 1.7%) as a yellow solid. ¹H NMR (400 MHz, CDCl₃) δ 8.30 (s, 1H), 7.31 (dd, J=14.5, 7.8 Hz, 2H), 7.19 (t, J=7.7 Hz, 1H), 7.01 (d, J=8.0 Hz, 2H), 6.94 (d, J=7.5 Hz, 2H), 6.36 (s, 1H), 6.24 (d, J=7.5 Hz, 1H), 4.33 (dd, J=17.1, 7.3 Hz, 2H), 4.23 (s, 2H), 4.05 (s, 2H), 3.93 (d, J=4.6 Hz, 2H), 3.75 (s, 1H), 3.39 (s, 2H), 2.93 (d, J=9.5 Hz, 1H), 2.86 (d, J=10.6 Hz, 1H), 2.73 (t, J=10.1 Hz, 2H), 2.46 (s, 3H), 1.79 (s, 2H). LC-MS m/z: 447.2 [M+H]⁺. HPLC Purity (214 nm): 100%; t_{R}=4.99 min.

### Example 357—2-(2-Morpholinoethoxy)-N-phenethyl-1H-benzo[d]imidazole-1-carboxamide

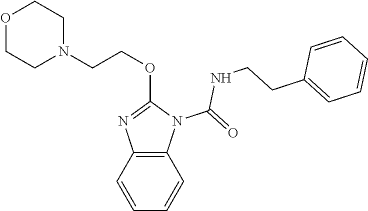 [SMILES: O=C(NCCc1ccccc1)n1c(OCCN2CCOCC2)nc2ccccc21]

Following general procedure E, 2-chloro-1-((2-(trimethylsilyl)ethoxy)methyl)-1H-benzo[d]imidazole (1.3 g, 4.6 mmol) and 2-morpholinoethan-1-ol (864 mg, 6.0 mmol) afforded 4-(2-((1-((2-(trimethylsilyl)ethoxy)methyl)-1H-benzo[d]imidazol-2-yl)oxy)ethyl)morpholine (1.0 g, 53.2%) as a colorless oil. LC-MS m/z: 378 [M+H]⁺. Purity (214 nm): 97.39%; t_{R}=1.64 min.

Following general procedure F, 4-(2-((1-((2-(trimethylsilyl)ethoxy)methyl)-1H-benzo[d]imidazol-2-yl)oxy)ethyl)morpholine (1.0 g, 2.7 mmol) afforded 4-(2-((1H-benzo[d]imidazol-2-yl)oxy)ethyl)morpholine (420 mg, 62.9%) as a yellow oil. LC-MS m/z: 248.2 [M+H]⁺. Purity (214 nm): 98.18%; t_{R}=1.53 min.

Following general procedure C, 4-(2-((1H-benzo[d]imidazol-2-yl)oxy)ethyl) morpholine (90 mg, 0.536 mmol) and 2-phenylethan-1-amine (43 mg, 0.36 mmol) afforded the title compound 23.1 mg, 16.1%) as a white solid. ¹H NMR (400 MHz, CDCl₃) δ 8.21-8.14 (m, 1H), 7.47 (ddd, J=11.5, 5.4, 3.0 Hz, 1H), 7.41 (t, J=5.4 Hz, 1H), 7.36-7.30 (m, 2H), 7.29-7.27 (m, 1H), 7.24 (s, 2H), 7.22 (dd, J=7.5, 2.1 Hz, 2H), 4.64 (dd, J=7.0, 4.2 Hz, 2H), 3.72 (dd, J=12.7, 6.8 Hz, 2H), 3.66-3.56 (m, 4H), 2.97 (t, J=6.8 Hz, 2H), 2.62 (t, J=5.6 Hz, 2H), 2.44-2.29 (m, 4H). LC-MS m/z: 395.1 [M+H]⁺. HPLC Purity (214 nm): 97.85%; t_{R}=5.47 min.

### Example 358—4-(6-Methyl-2,6-diazaspiro[3.3]heptan-2-yl)-N-(4-methylpent-2-yn-1-yl)-1H-benzo[d]imidazole-1-carboxamide

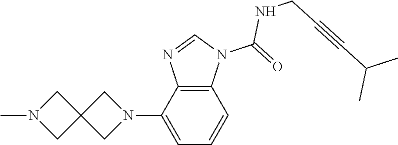 [SMILES: CC(C)C#CCNC(=O)n1cnc2c(N3CC4(CN(C)C4)C3)cccc21]

Following general procedure E, 3-fluoro-2-nitroaniline (640 mg, 4.1 mmol) and tert-butyl 2,6-diazaspiro[3.3]heptane-2-carboxylate (1 g, 4.1 mmol) afforded tert-butyl 6-(3-amino-2-nitrophenyl)-2,6-diazaspiro[3.3]heptane-2-carboxylate (864 mg, 63.1%) as a red solid. LC-MS m/z: 335.0 [M+H]⁺. Purity (214 nm): 93.84%; t_{R}=2.03 min.

Following general procedure F, tert-butyl 6-(3-amino-2-nitrophenyl)-2,6-diazaspiro[3.3]heptane-2-carboxylate (864 g, 2.6 mmol) afforded tert-butyl 6-(2,3-diaminophenyl)-2,6-diazaspiro[3.3]heptane-2-carboxylate (661 mg, 83.3%) as a brown solid. LC-MS m/z: 305.3 [M+H]⁺. Purity (214 nm): 96.36%; t_{R}=1.82 min.

Following general procedure F (method A), tert-butyl 6-(2,3-diaminophenyl)-2,6-diazaspiro[3.3]heptane-2-carboxylate (640 mg, 2.4 mmol) afforded tert-butyl 6-(1H-benzo[d]imidazol-4-yl)-2,6-diazaspiro[3.3]heptane-2-carboxylate (575 mg, 75.9%) as a red solid. LC-MS m/z: 315.3 [M+H]⁺. Purity (214 nm): 100%; t_{R}=1.85 min.

A mixture of tert-butyl 6-(1H-benzo[d]imidazol-4-yl)-2,6-diazaspiro[3.3]heptane-2-carboxylate (545 mg, 1.7 mmol) and TFA (3 mL) was stirred at RT for 1 h. The mixture was concentrated to afford crude 4-(2,6-diazaspiro[3.3]heptan-2-yl)-1H-benzo[d]imidazole (180 mg, 50%) as a yellow oil. LC-MS m/z: 215.0 [M+H]⁺. Purity (254 nm): 92.49%; t_{R}=0.84 min.

To a solution of 4-(2,6-diazaspiro[3.3]heptan-2-yl)-1H-benzo[d]imidazole (crude) (180 mg, 1.7 mmol) in CH₃OH (20 mL) was added tert-butyl HCHO (194 mg, 2.4 mmol). The reaction mixture was stirred at RT for 30 min and then NaBH₃CN (321 mg, 5.1 mmol) was added. The resulting mixture was stirred at RT for 16 h. The mixture was concentrated and purified by silica gel column chromatography (DCM:MeOH=10:1) to give 4-(6-methyl-2,6-diazaspiro[3.3]heptan-2-yl)-1H-benzo[d]imidazole (20 mg, 10%) as a green solid. LC-MS m/z: 229.2 [M+H]⁺. Purity (214 nm): 85.57%; t_{R}=1.26 min.

Following general procedure C, 4-(6-methyl-2,6-diazaspiro[3.3]heptan-2-yl)-1H-benzo[d]imidazole (20 mg, 0.09 mmol) and 4-methylpent-2-yn-1-amine (9 mg, 0.09 mmol) afforded the title compound (3.8 mg, 12%) as a white solid. ¹H NMR (400 MHz, CDCl₃) δ 8.49 (s, 1H), 8.29 (s, 1H), 7.22 (d, J=7.9 Hz, 1H), 7.17 (d, J=7.4 Hz, 1H), 6.27 (d, J=7.7 Hz, 1H), 6.16 (s, 1H), 4.37 (s, 4H), 4.27 (dd, J=5.1, 2.0 Hz, 2H), 4.03 (d, J=19.7 Hz, 4H), 2.67 (s, 4H), 1.17 (t, J=9.0 Hz, 6H). LC-MS m/z: 352.3 [M+H]⁺. HPLC Purity (214 nm): 96.20%; t_{R}=7.52 min.

### Example 359—4-(1-Methyl-1,6-diazaspiro[3.3]heptan-6-yl)-N-(4-methylpent-2-yn-1-yl)-1H-benzo[d]imidazole-1-carboxamide

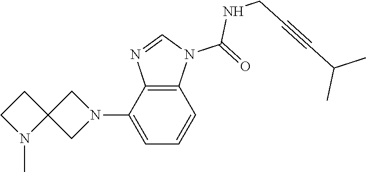 [SMILES: CC(C)C#CCNC(=O)n1cnc2c(N3CC4(CCN4C)C3)cccc21]

Following general procedure E, 3-fluoro-2-nitroaniline (468 mg, 3.0 mmol) and 1-methyl-1,6-diazaspiro[3.3]heptane (336 mg, 3.0 mmol) afforded 3-(1-methyl-1,6-diazaspiro[3.3]heptan-6-yl)-2-nitroaniline (510 mg, crude) as a yellow solid. LC-MS m/z: 249.2 [M+H]⁺. Purity (214 nm): 78%; t_{R}=1.59 min.

Following general procedure F, 3-(1-methyl-1,6-diazaspiro[3.3]heptan-6-yl)-2-nitroaniline (500 mg, 2.0 mmol) afforded 3-(1-methyl-1,6-diazaspiro[3.3]heptan-6-yl)benzene-1,2-diamine (440 mg, 100%) as a yellow oil. LC-MS m/z: 219.2 [M+H]⁺. Purity (214 nm): 73%; t_{R}=1.25 min.

Following general procedure G (method A), 3-(1-methyl-1,6-diazaspiro[3.3]heptan-6-yl)benzene-1,2-diamine (436 mg, 2.0 mmol) afforded 4-(1-methyl-1,6-diazaspiro[3.3]heptan-6-yl)-1H-benzo[d]imidazole (250 mg, 55%) as a yellow oil. LC-MS m/z: 229.3 [M+H]⁺. Purity (214 nm): 86%; t_{R}=1.30 min.

Following general procedure C, 4-(1-methyl-1,6-diazaspiro[3.3]heptan-6-yl)-1H-benzo[d]imidazole (88.0 mg, 0.4 mmol) and 4-methylpent-2-yn-1-amine (67.0 mg, 0.5 mmol) afforded the title compound (6.5 mg, 5%) as a yellow oil. ¹H NMR (400 MHz, CDCl₃) δ 8.35 (s, 1H), 8.30 (s, 1H), 7.23 (s, 1H), 7.15 (d, J=8.5 Hz, 1H), 6.30 (d, J=7.5 Hz, 1H), 5.98 (s, 1H), 4.54 (d, J=9.3 Hz, 2H), 4.35-4.17 (m, 4H), 3.47 (t, J=7.0 Hz, 2H), 2.64-2.57 (m, 6H), 1.18 (d, J=6.7 Hz, 6H). LC-MS m/z: 352.3 [M+H]⁺. HPLC Purity (214 nm): 99.41%; t_{R}=7.57 min.

### Example 360—N-(4,4-Dimethylpent-2-yn-1-yl)-4-(3-(4-methylpiperazin-1-yl)azetidin-1-yl)-1H benzo[d]imidazole-1-carboxamide

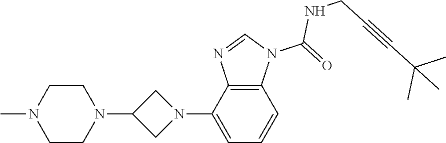 [SMILES: CN1CCN(C2CN(c3cccc4c3ncn4C(=O)NCC#CC(C)(C)C)C2)CC1]

Following general procedure C, 4-(3-(4-methylpiperazin-1-yl)azetidin-1-yl)-1H-benzo[d]imidazole (0.15 g, 0.55 mmol) and 4,4-dimethylpent-2-yn-1-amine hydrochloride (0.12 g, 0.83 mmol) afforded the title compound (88.5 mg, 31.2%) as a white solid. ¹H NMR (400 MHz, DMSO-d₆) δ 8.88 (bs, 1H), 8.49 (s, 1H), 7.36 (d, J=7.6 Hz, 1H), 7.13 (t, J=8.0 Hz, 1H), 6.20 (d, J=7.2 Hz, 1H), 4.21 (t, J=7.2 Hz, 2H), 4.10 (d, J=5.2 Hz, 2H), 3.86-3.83 (m, 2H), 3.25 (t, J=6.0 Hz, 1H), 2.35 (br, 8H), 2.25 (s, 3H), 1.19 (s, 9H). LC-MS: m/z: 409.2 [M+H]⁺. HPLC Purity (214 nm): 94%; t_{R}=6.21 min.

### Example 361—4-(4-(4-Methylazetidin-3-yl)piperazin-1-yl)-N-(4-phenylbut-3-yn-1-yl)-1H-benzo[d]imidazole-1-carboxamide

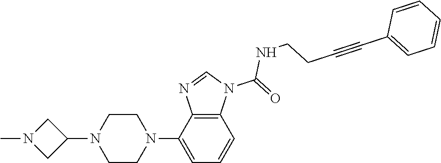 [SMILES: CN1CC(N2CCN(c3cccc4c3ncn4C(=O)NCCC#Cc3ccccc3)CC2)C1]

Following general procedure C, 4-(3-(4-methylpiperazin-1-yl)azetidin-1-yl)-1H-benzo[d]imidazole (135 mg, 0.5 mmol) and 4-phenylbut-3-yn-1-amine (73 mg, 0.5 mmol) afforded the title compound (13.5 mg, 6.1%) as a white solid. ¹H NMR (400 MHz, CDCl₃) δ 8.55 (s, 1H), 8.44 (s, 1H), 7.49 (d, J=8.2 Hz, 1H), 7.41 (dd, J=7.1, 2.6 Hz, 2H), 7.34-7.28 (m, 3H), 7.17 (t, J=8.1 Hz, 1H), 6.85 (s, 1H), 6.69 (d, J=7.9 Hz, 1H), 4.13 (t, J=8.1 Hz, 2H), 3.75 (dd, J=12.1, 6.1 Hz, 2H), 3.54 (s, 4H), 3.50-3.42 (m, 2H), 3.34 (dd, J=13.8, 6.7 Hz, 2H), 2.84 (t, J=6.3 Hz, 2H), 2.70 (s, 3H), 2.60-2.53 (m, 4H). LC-MS m/z: 443.1 [M+H]⁺. HPLC Purity (214 nm): 100%; t_{R}=6.01 min.

### Example 362—4-(3-(4-Methylpiperazin-1-yl)azetidin-1-yl)-N-(4-phenylbut-3-ynyl)-1H-benzo[d]imidazole-1-carboxamide

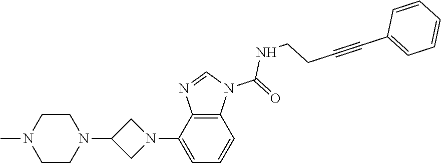 [SMILES: CN1CCN(C2CN(c3cccc4c3ncn4C(=O)NCCC#Cc3ccccc3)C2)CC1]

Following general procedure C. 4-(3-(4-methylpiperazin-1-yl)azetidin-1-yl)-1H-benzo[d]imidazole (0.15 g, 0.55 mmol) and 4-phenylbut-3-yn-1-amine (0.12 g, 0.83 mmol) afforded the title compound (59.2 mg, 24.3%) as a white solid. ¹H NMR (400 MHz, CDCl₃) δ 8.55 (s, 1H), 7.43-7.40 (m, 2H), 7.32-7.30 (m, 3H), 7.14-7.07 (m, 2H), 6.35 (br, 1H), 6.23 (d, J=7.6 Hz, 1H), 4.35 (t, J=6.8 Hz, 2H), 4.05 (m, 2H), 3.74 (q, J=6.0 Hz, 2H), 3.40 (m, 1H), 2.85-2.61 (m, 10H), 2.48 (s, 3H). LC-MS: m/z: 443.2 [M+H]⁺. HPLC Purity (214 nm): 97%; t_{R}=6.24 min.

### Example 363—2-(2-(4-Methylpiperazin-1-yl)ethoxy)-N-(4-phenylbut-3-yn-1-yl)-1H-benzo[d]imidazole-1-carboxamide

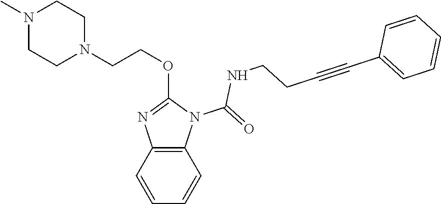 [SMILES: CN1CCN(CCOc2nc3ccccc3n2C(=O)NCCC#Cc2ccccc2)CC1]

Following general procedure C, to a solution of 2-(2-(4-methylpiperazin-1-yl)ethoxy)-1H-benzo[d]imidazole (260 mg, 1.0 mmol) and 4-phenylbut-3-yn-1-amine (145 mg, 1.0 mmol) afforded the title compound (30.2 mg, 7%) as a white solid. ¹H NMR (400 MHz, CDCl₃) δ 8.23-8.06 (m, 1H), 7.77 (t, J=5.5 Hz, 1H), 7.57-7.44 (m, 1H), 7.44-7.35 (m, 2H), 7.33-7.27 (m, 3H), 7.26-7.22 (m, 2H), 4.71 (t, J=5.6 Hz, 2H), 3.69 (q, J=6.5 Hz, 2H), 2.82 (t, J=5.6 Hz, 2H), 2.76 (t, J=5.6 Hz, 2H), 2.63-2.30 (m, 8H), 2.27 (s, 3H). LC-MS m/z: 432.4 [M+H]⁺. HPLC Purity (214 nm): 98.28%; t_{R}=6.97 min.

### Example 364—4-(3-(Dimethylamino)azetidin-1-yl)-N-(4-phenylbut-3-ynyl)-1H-benzo[d]imidazole-1-carboxamide

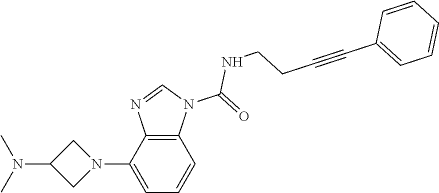 [SMILES: CN(C)C1CN(c2cccc3c2ncn3C(=O)NCCC#Cc2ccccc2)C1]

Following general procedure C, 1-(1H-benzo[d]imidazol-4-yl)-N,N-dimethylazetidin-3-amine (0.10 g, 0.46 mmol) and 4-phenylbut-3-yn-1-amine (0.10 g, 0.69 mmol) afforded the title compound (50.1 mg, 27.9%) as a white solid. ¹H NMR (400 MHz, DMSO-d₆) δ 8.75 (bs, 1H), 8.74 (s, 1H), 7.40-7.33 (m, 6H), 7.11 (t, J=6.4 Hz, 1H), 6.20 (d, J=6.0 Hz, 1H), 4.20 (t, J=6.0 Hz, 2H), 3.81 (m, 2H), 3.51 (q, J=5.6 Hz, 2H), 3.18-3.16 (m, 1H), 2.75 (t, J=6.0 Hz, 2H), 2.10 (s, 6H). LC-MS: m/z: 388.0 [M+H]⁺. HPLC Purity (214 nm): 100%; t_{R}=5.54 min.

### Example 365—N-((2,3-Dihydrobenzo[b][1,4]dioxin-6-yl)methyl)-4-(4-methylpiperazin-1-yl)-1H-benzo[d]imidazole-1-carboxamide

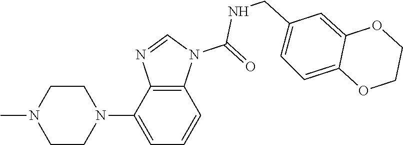 [SMILES: CN1CCN(c2cccc3c2ncn3C(=O)NCc2ccc3c(c2)OCCO3)CC1]

Following general procedure C, to a solution of 4-(4-methylpiperazin-1-yl)-1H-benzo[d]imidazole (0.15 g, 0.69 mmol) and (2,3-dihydrobenzo[b][1,4]dioxin-6-yl)-methanamine (0.17 g, 1.04 mmol) afforded the title compound (31.6 mg, 11.2%) as a white solid. ¹H NMR (400 MHz, CDCl₃) δ 8.40 (s, 1H), 7.38 (d, J=6.4 Hz, 1H), 7.27-7.24 (m, 1H), 6.91 (s, 1H), 6.87 (s, 2H), 6.72 (d, J=6.8 Hz, 1H), 6.38 (bs, 1H), 4.55 (d, J=4.4 Hz, 2H), 4.24 (s, 4H), 3.65 (bs, 4H), 3.03 (bs, 4H), 2.10 (s, 3H). LC-MS: m/z: 408.1 [M+H]⁺. HPLC Purity (214 nm): 100%; t_{R}=5.62 min.

### Example 366—4-(7-Methyl-2,7-diazaspiro[3.5]nonan-2-yl)-N-(4-phenylbut-3-yn-1-yl)-1H-benzo[d]imidazole-1-carboxamide

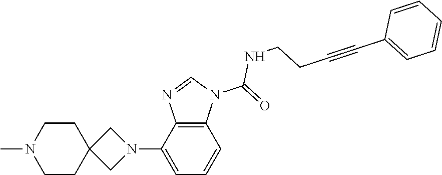 [SMILES: CN1CCC2(CC1)CN(c1cccc3c1ncn3C(=O)NCCC#Cc1ccccc1)C2]

Following general procedure C, to a solution of 2-(1H-benzo[d]imidazol-4-yl)-7-methyl-2,7-diazaspiro[3.5]nonane (75 mg, 0.29 mmol) and 4-phenylbut-3-yn-1-amine (52 mg, 0.29 mmol) afforded the title compound (21.7 mg, 17.6%) as a white solid. ¹H NMR (400 MHz, CDCl₃) δ 8.36 (s, 1H), 7.43-7.41 (m, 2H), 7.32-7.27 (m, 3H), 7.18 (d, J=8.0 Hz, 1H), 7.10 (t, J=8.0 Hz, 1H), 6.70 (t, J=5.6 Hz, 1H), 6.22 (d, J=7.6 Hz, 1H), 4.01 (s, 4H), 3.75 (q, J=6.0 Hz, 2H), 2.92-2.82 (m, 6H), 2.55 (s, 3H), 2.10 (t, J=5.6 Hz, 4H). LC-MS m/z: 428.3 [M+H]⁺. HPLC Purity (214 nm): 99.52%; t_{R}=5.69 min.

### Example 367—4-(2-Methyl-2,7-diazaspiro[3.5]nonan-7-yl)-N-(4-phenylbut-3-yn-1-yl)-1H-benzo[d]imidazole-1-carboxamide

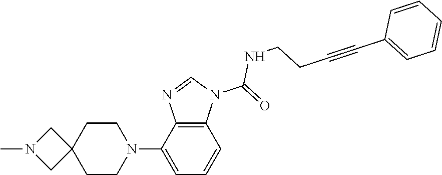 [SMILES: CN1CC2(CCN(c3cccc4c3ncn4C(=O)NCCC#Cc3ccccc3)CC2)C1]

Following general procedure C, 7-(1H-benzo[d]imidazol-4-yl)-2-methyl-2,7-diazaspiro[3.5]nonane (100 mg, 0.39 mmol) and 4-phenylbut-3-yn-1-amine (70 mg, 0.39 mmol) afforded the title compound (17.3 mg, 10.4%) as a white solid. ¹H NMR (400 MHz, CDCl₃) δ 8.42 (s, 1H), 7.45 (d, J=7.6 Hz, 1H), 7.43-7.40 (m, 2H), 7.33-7.26 (m, 3H), 7.15 (t, J=7.6 Hz, 1H), 6.80 (t, J=5.2 Hz, 1H), 6.68 (d, J=7.6 Hz, 1H), 3.75 (q, J=6.4 Hz, 2H), 3.68 (s, 4H), 3.38 (t, J=4.8 Hz, 4H), 2.84 (t, J=6.4 Hz, 2H), 2.73 (s, 3H), 2.06 (t, J=5.2 Hz, 4H). LC-MS m/z: 428.1 [M+H]⁺. HPLC Purity (214 nm): 100.00%; t_{R}=6.23 min.

### Other Exemplary Compounds

Compound Nos. 97a, 107a, 112a, 112b, 221a, and 221b of Table 1 were prepared by procedures similar to those described above with the appropriate substitution for one or more of the reagents.

### Example B—Biological Activity Evaluation

The ability of exemplary compounds to inhibit acid ceramidase was measured. Experimental procedures and results are provided below.

### Part I: Assay Procedure

Cell lysates overexpressing acid ceramidase were used as the enzyme source for compound potency determination in a biochemical fluorescent assay. Briefly, compounds were preincubated with 10 μg protein of cell lysates in a dose-response manner for 1 hr at RT in the assay buffer containing 25 mM NaAC and 100 mM NaCl, pH 4.5. The reaction was initiated by the addition of substrate Rbm14-12 at a final concentration of 6.3 μM. The reaction was run at RT for 1 hr before it was stopped by the addition of the stopping buffer containing 20% methanol (v/v), 1 mg/ml NaIO₄, 0.1 M glycine, pH 10.6. The samples were incubated with the stopping buffer at RT for 1 hr to allow the fluorescent product to be formed. Finally, the plate was read with SpectraMax i3 plate reader (Molecular Devices) at ex360 nm and em446 nm. Data were collected and used to determine the IC₅₀ values of compounds by curve fitting to the four-parameter inhibition equation.

### Part II: Results

Acid ceramidse inhibition values for tested compounds are provided in Table 3 below, along with cLogP and compound solubility in water. The symbol “A” indicates inhibition of less than 0.2 μM; the symbol “B” indicates inhibition in the range of 0.2 μM up to 1 μM; and the symbol “C” indicates inhibition of greater than 1 μM.

TABLE 1

| | | | Compound | |
|---|---|---|---|---|
| | | | Solubility | |
| Example | | | in Water | IC₅₀ |
| No. | Compound Structure | cLogP | (μg/mL) | hACR |
| 1 | 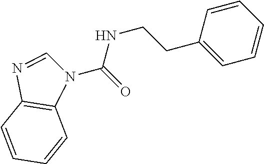 [SMILES: O=C(NCCc1ccccc1)n1cnc2ccccc21] | 2.1 | 8.0 | A |
| 2 | 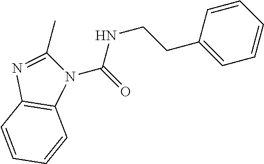 [SMILES: Cc1nc2ccccc2n1C(=O)NCCc1ccccc1] | 2.4 | 0 | A |
| 3 | 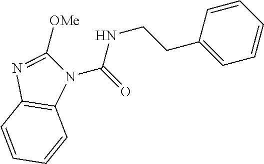 [SMILES: COc1nc2ccccc2n1C(=O)NCCc1ccccc1] | 2.9 | 10.0 | A |
| 4 | 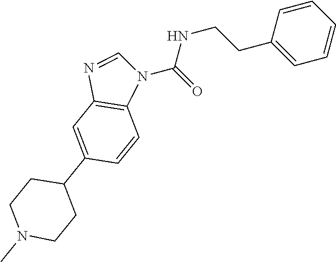 [SMILES: CN1CCC(c2ccc3c(c2)ncn3C(=O)NCCc2ccccc2)CC1] | 2.8 | 20.6 | A |
| 5 | 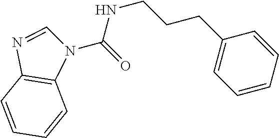 [SMILES: O=C(NCCCc1ccccc1)n1cnc2ccccc21] | 2.5 | 13.8 | A |
| 6 | 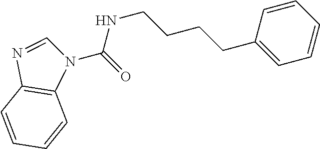 [SMILES: O=C(NCCCCc1ccccc1)n1cnc2ccccc21] | 3.1 | 1.0 | A |
| 7a | 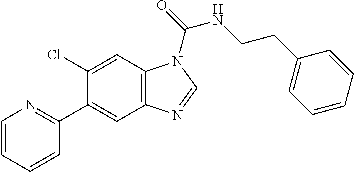 [SMILES: O=C(NCCc1ccccc1)n1cnc2cc(-c3ccccn3)c(Cl)cc21] | 3.2 | 0.4 | A |
| 7b | 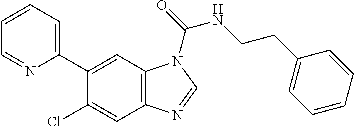 [SMILES: O=C(NCCc1ccccc1)n1cnc2cc(Cl)c(-c3ccccn3)cc21] | 3.2 | 0.1 | A |
| 8a | 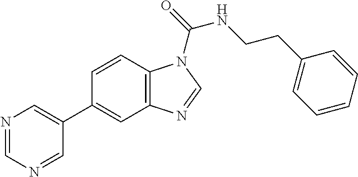 [SMILES: O=C(NCCc1ccccc1)n1cnc2cc(-c3cncnc3)ccc21] | 1.6 | 3.7 | A |
| 8b | 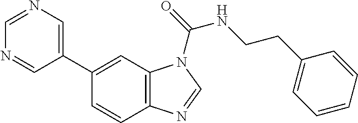 [SMILES: O=C(NCCc1ccccc1)n1cnc2ccc(-c3cncnc3)cc21] | 1.6 | 14.5 | A |
| 9 | 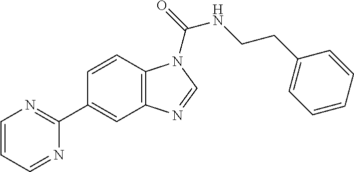 [SMILES: O=C(NCCc1ccccc1)n1cnc2cc(-c3ncccn3)ccc21] | 1.8 | 5.4 | A |
| 10a | 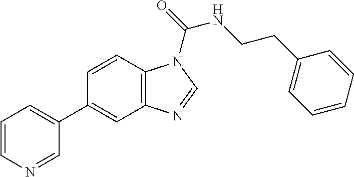 [SMILES: O=C(NCCc1ccccc1)n1cnc2cc(-c3cccnc3)ccc21] | 2.5 | 1.2 | A |
| 10b | 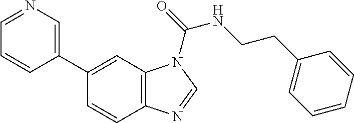 [SMILES: O=C(NCCc1ccccc1)n1cnc2ccc(-c3cccnc3)cc21] | 2.5 | 0.6 | A |
| 11a | 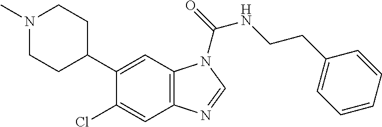 [SMILES: CN1CCC(c2cc3c(cc2Cl)ncn3C(=O)NCCc2ccccc2)CC1] | 3.5 | 23.1 | A |
| 11b | 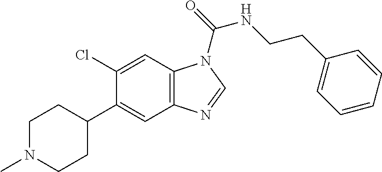 [SMILES: CN1CCC(c2cc3ncn(C(=O)NCCc4ccccc4)c3cc2Cl)CC1] | 3.5 | 23.1 | A |
| 12 | 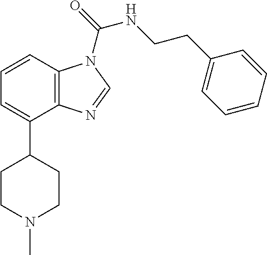 [SMILES: CN1CCC(c2cccc3c2ncn3C(=O)NCCc2ccccc2)CC1] | 3.5 | 30.5 | A |
| 13a | 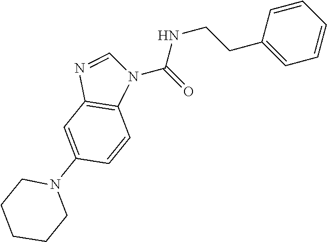 [SMILES: O=C(NCCc1ccccc1)n1cnc2cc(N3CCCCC3)ccc21] | 3.4 | 2.6 | A |
| 13b | 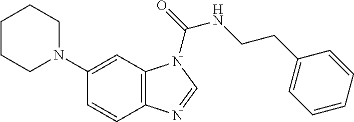 [SMILES: O=C(NCCc1ccccc1)n1cnc2ccc(N3CCCCC3)cc21] | 3.4 | 9.1 | A |
| 14a | 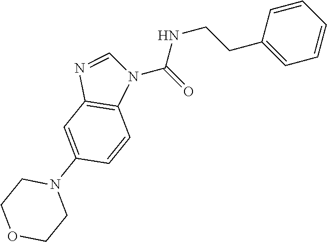 [SMILES: O=C(NCCc1ccccc1)n1cnc2cc(N3CCOCC3)ccc21] | 2.0 | 16.1 | A |
| 14b | 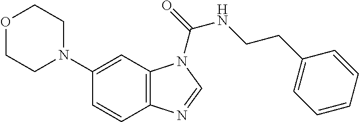 [SMILES: O=C(NCCc1ccccc1)n1cnc2ccc(N3CCOCC3)cc21] | 2.0 | 31.2 | A |
| 15a | 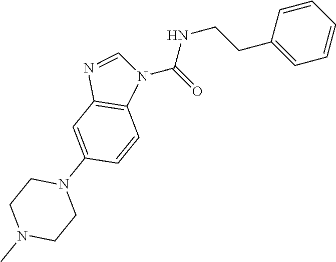 [SMILES: CN1CCN(c2ccc3c(c2)ncn3C(=O)NCCc2ccccc2)CC1] | 2.6 | 20.4 | A |
| 15b | 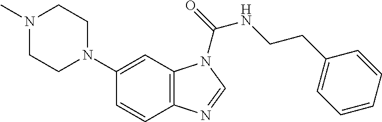 [SMILES: CN1CCN(c2ccc3ncn(C(=O)NCCc4ccccc4)c3c2)CC1] | 2.6 | 22.9 | A |
| 16a | 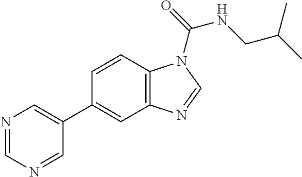 [SMILES: CC(C)CNC(=O)n1cnc2cc(-c3cncnc3)ccc21] | 0.9 | 4.2 | A |
| 16b | 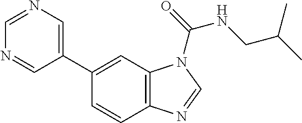 [SMILES: CC(C)CNC(=O)n1cnc2ccc(-c3cncnc3)cc21] | 0.9 | 18.3 | A |
| 17 | 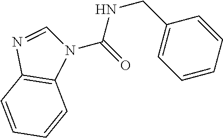 [SMILES: O=C(NCc1ccccc1)n1cnc2ccccc21] | 1.9 | 1.9 | A |
| 18 | 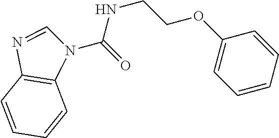 [SMILES: O=C(NCCOc1ccccc1)n1cnc2ccccc21] | 2.2 | 2.0 | A |
| 19a | 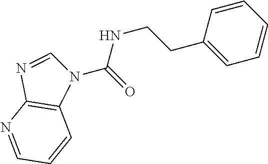 [SMILES: O=C(NCCc1ccccc1)n1cnc2ncccc21] | 1.0 | 26.1 | A |
| 20 | 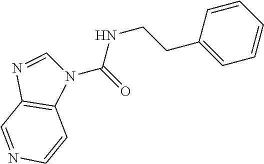 [SMILES: O=C(NCCc1ccccc1)n1cnc2cnccc21] | 1.0 | 5.3 | A |
| 21 | 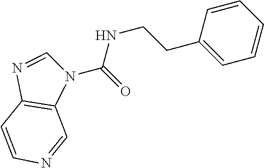 [SMILES: O=C(NCCc1ccccc1)n1cnc2ccncc21] | 1.0 | 4.3 | A |
| 22 | 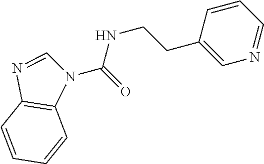 [SMILES: O=C(NCCc1cccnc1)n1cnc2ccccc21] | 0.7 | 12.9 | B |
| 23 | 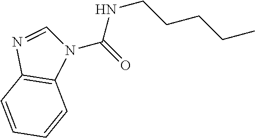 [SMILES: CCCCCNC(=O)n1cnc2ccccc21] | 2.2 | 13.3 | A |
| 24 | 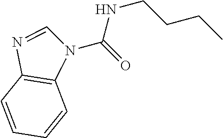 [SMILES: CCCCNC(=O)n1cnc2ccccc21] | 1.6 | 21.9 | A |
| 25a | 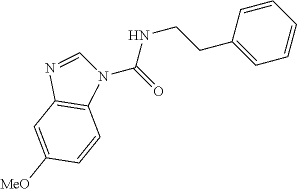 [SMILES: COc1ccc2c(c1)ncn2C(=O)NCCc1ccccc1] | 2.4 | 2.6 | A |
| 26a | 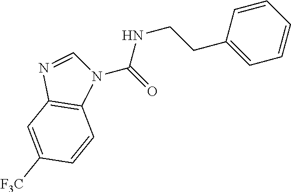 [SMILES: O=C(NCCc1ccccc1)n1cnc2cc(C(F)(F)F)ccc21] | 3.1 | 0.5 | A |
| 26b | 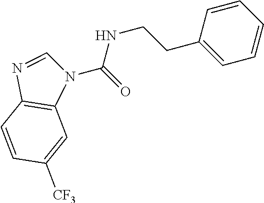 [SMILES: O=C(NCCc1ccccc1)n1cnc2ccc(C(F)(F)F)cc21] | 3.1 | 2.6 | A |
| 19b | 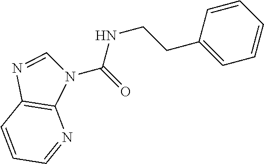 [SMILES: O=C(NCCc1ccccc1)n1cnc2cccnc21] | 1.0 | 24.5 | A |
| 27 | 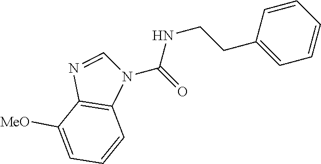 [SMILES: COc1cccc2c1ncn2C(=O)NCCc1ccccc1] | 2.4 | 7.8 | A |
| 25b | 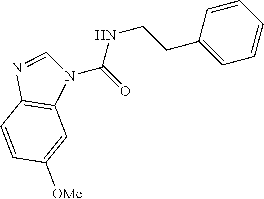 [SMILES: COc1ccc2ncn(C(=O)NCCc3ccccc3)c2c1] | 2.4 | 15.0 | A |
| 28 & 29a | 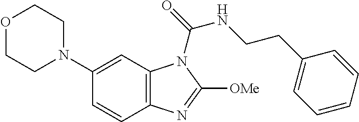 [SMILES: COc1nc2ccc(N3CCOCC3)cc2n1C(=O)NCCc1ccccc1] | 2.8 | 18.0 | B |
| 29b | 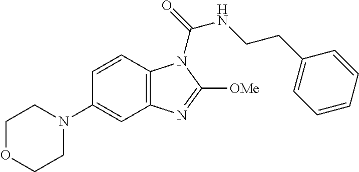 [SMILES: COc1nc2cc(N3CCOCC3)ccc2n1C(=O)NCCc1ccccc1] | 2.8 | 3.6 | B |
| 30a | 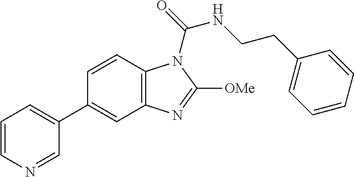 [SMILES: COc1nc2cc(-c3cccnc3)ccc2n1C(=O)NCCc1ccccc1] | 3.3 | 1.6 | A |
| 30b | 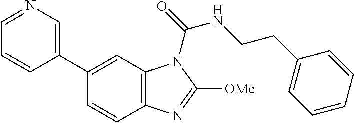 [SMILES: COc1nc2ccc(-c3cccnc3)cc2n1C(=O)NCCc1ccccc1] | 3.3 | 3.9 | A |
| 31a | 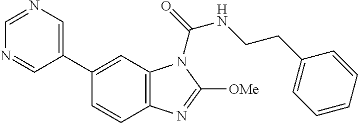 [SMILES: COc1nc2ccc(-c3cncnc3)cc2n1C(=O)NCCc1ccccc1] | 2.4 | 7.1 | A |
| 32a | 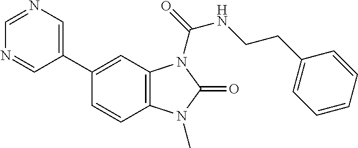 [SMILES: Cn1c(=O)n(C(=O)NCCc2ccccc2)c2cc(-c3cncnc3)ccc21] | 3.1 | 0.9 | C |
| 32b | 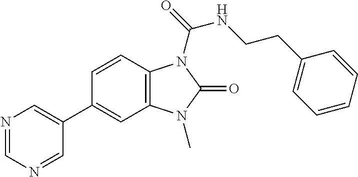 [SMILES: Cn1c(=O)n(C(=O)NCCc2ccccc2)c2ccc(-c3cncnc3)cc21] | 3.1 | 0.3 | C |
| 31b | 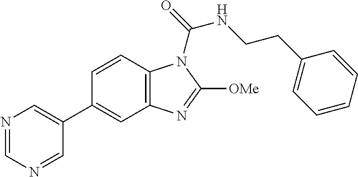 [SMILES: COc1nc2cc(-c3cncnc3)ccc2n1C(=O)NCCc1ccccc1] | 2.4 | 8.6 | A |
| 33 | 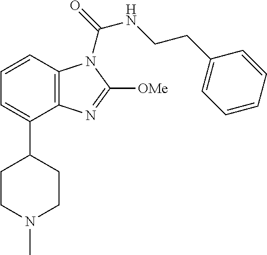 [SMILES: COc1nc2c(C3CCN(C)CC3)cccc2n1C(=O)NCCc1ccccc1] | 3.4 | 27.0 | B |
| 34 | 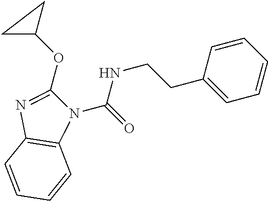 [SMILES: O=C(NCCc1ccccc1)n1c(OC2CC2)nc2ccccc21] | 3.5 | 0.5 | A |
| 35 | 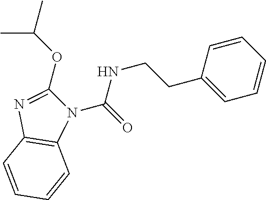 [SMILES: CC(C)Oc1nc2ccccc2n1C(=O)NCCc1ccccc1] | 3.8 | 0.7 | A |
| 36 | 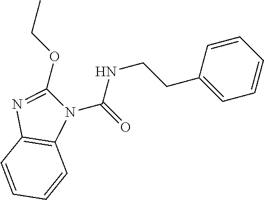 [SMILES: CCOc1nc2ccccc2n1C(=O)NCCc1ccccc1] | 3.4 | 8.4 | A |
| 37 | 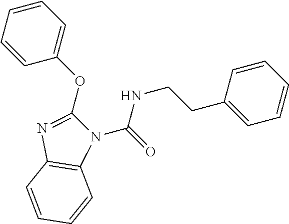 [SMILES: O=C(NCCc1ccccc1)n1c(Oc2ccccc2)nc2ccccc21] | 4.2 | 0.2 | A |
| 38a | 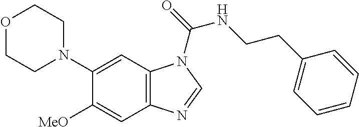 [SMILES: COc1cc2ncn(C(=O)NCCc3ccccc3)c2cc1N1CCOCC1] | 2.3 | 20.4 | A |
| 38b | 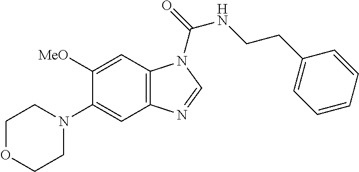 [SMILES: COc1cc2c(cc1N1CCOCC1)ncn2C(=O)NCCc1ccccc1] | 2.3 | 18.3 | A |
| 39 | 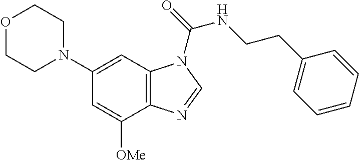 [SMILES: COc1cc(N2CCOCC2)cc2c1ncn2C(=O)NCCc1ccccc1] | 2 3 | 16.3 | A |
| 40 | 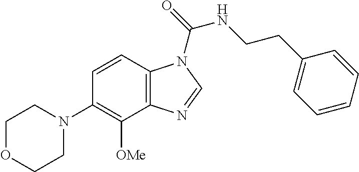 [SMILES: COc1c(N2CCOCC2)ccc2c1ncn2C(=O)NCCc1ccccc1] | 2.2 | 10.5 | A |
| 41 | 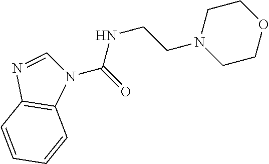 [SMILES: O=C(NCCN1CCOCC1)n1cnc2ccccc21] | 0.6 | 32.0 | C |
| 42 | 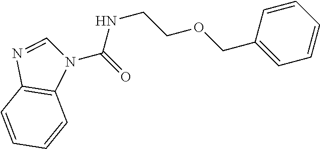 [SMILES: O=C(NCCOCc1ccccc1)n1cnc2ccccc21] | 2.2 | 17.7 | A |
| 43 | 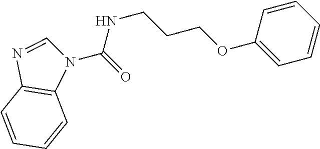 [SMILES: O=C(NCCCOc1ccccc1)n1cnc2ccccc21] | 2.6 | 3.5 | A |
| 44 | 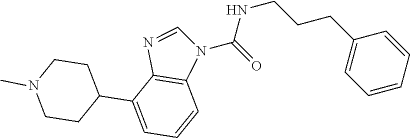 [SMILES: CN1CCC(c2cccc3c2ncn3C(=O)NCCCc2ccccc2)CC1] | 3.2 | 23.2 | A |
| 45 | 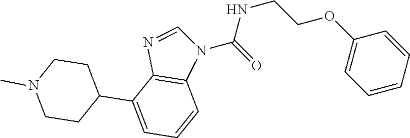 [SMILES: CN1CCC(c2cccc3c2ncn3C(=O)NCCOc2ccccc2)CC1] | 2.9 | 23.5 | A |
| 46a | 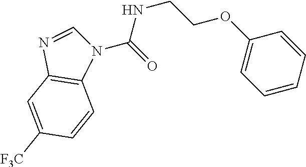 [SMILES: O=C(NCCOc1ccccc1)n1cnc2cc(C(F)(F)F)ccc21] | 3.1 | 0.4 | A |
| 46b | 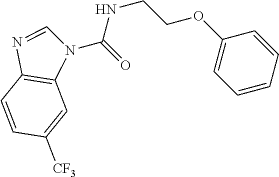 [SMILES: O=C(NCCOc1ccccc1)n1cnc2ccc(C(F)(F)F)cc21] | 3.2 | 0.3 | A |
| 47 | 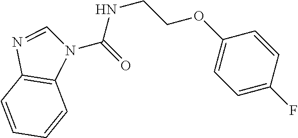 [SMILES: O=C(NCCOc1ccc(F)cc1)n1cnc2ccccc21] | 2.5 | 4.3 | A |
| 48 | 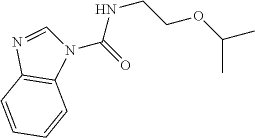 [SMILES: CC(C)OCCNC(=O)n1cnc2ccccc21] | 1.1 | 15.4 | B |
| 49a | 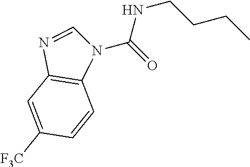 [SMILES: CCCCNC(=O)n1cnc2cc(C(F)(F)F)ccc21] | 2.6 | 15.6 | A |
| 50 | 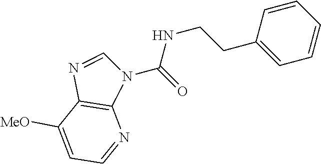 [SMILES: COc1ccnc2c1ncn2C(=O)NCCc1ccccc1] | 1.4 | 9.5 | B |
| 49b | 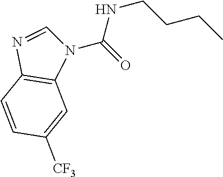 [SMILES: CCCCNC(=O)n1cnc2ccc(C(F)(F)F)cc21] | 2.6 | 12.6 | A |
| 51b | 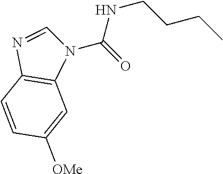 [SMILES: CCCCNC(=O)n1cnc2ccc(OC)cc21] | 1.9 | 8.5 | A |
| 52 | 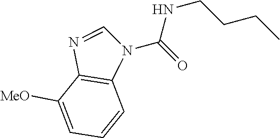 [SMILES: CCCCNC(=O)n1cnc2c(OC)cccc21] | 1.9 | 23.2 | A |
| 51a | 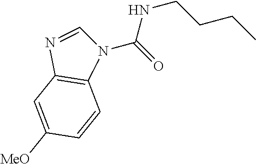 [SMILES: CCCCNC(=O)n1cnc2cc(OC)ccc21] | 1.9 | 7.2 | A |
| 53 | 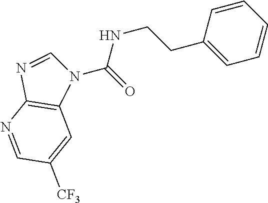 [SMILES: O=C(NCCc1ccccc1)n1cnc2ncc(C(F)(F)F)cc21] | 1.9 | 4.7 | A |
| 66a | 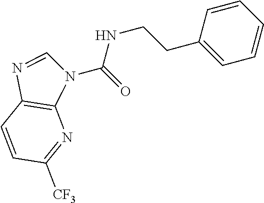 [SMILES: O=C(NCCc1ccccc1)n1cnc2ccc(C(F)(F)F)nc21] | 1.9 | 6.0 | A |
| 54 | 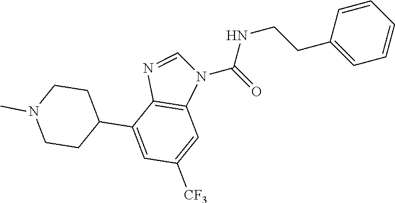 [SMILES: CN1CCC(c2cc(C(F)(F)F)cc3c2ncn3C(=O)NCCc2ccccc2)CC1] | 3.7 | 22.7 | A |
| 55 | 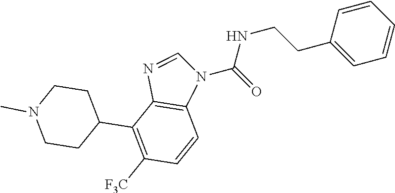 [SMILES: CN1CCC(c2c(C(F)(F)F)ccc3c2ncn3C(=O)NCCc2ccccc2)CC1] | 3.7 | 13 | A |
| 56 | 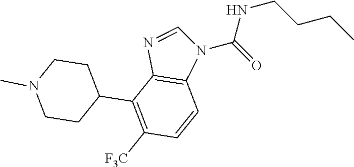 [SMILES: CCCCNC(=O)n1cnc2c(C3CCN(C)CC3)c(C(F)(F)F)ccc21] | 3.0 | 28.1 | C |
| 57 | 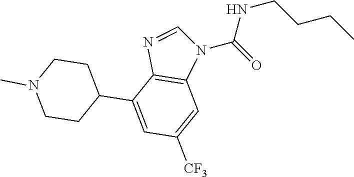 [SMILES: CCCCNC(=O)n1cnc2c(C3CCN(C)CC3)cc(C(F)(F)F)cc21] | 3.0 | 24.1 | A |
| 58 | 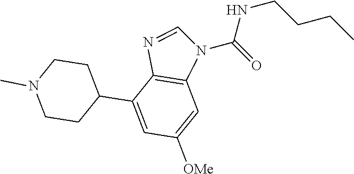 [SMILES: CCCCNC(=O)n1cnc2c(C3CCN(C)CC3)cc(OC)cc21] | 2.3 | 28.1 | B |
| 59 | 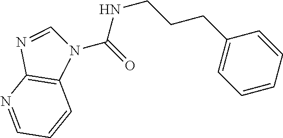 [SMILES: O=C(NCCCc1ccccc1)n1cnc2ncccc21] | 1.3 | 24.6 | A |
| 61 | 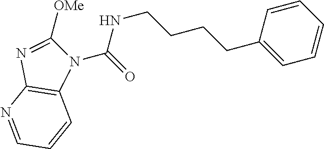 [SMILES: COc1nc2ncccc2n1C(=O)NCCCCc1ccccc1] | 2.8 | 1.0 | A |
| 62 | 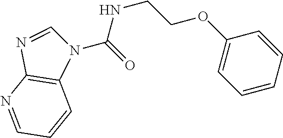 [SMILES: O=C(NCCOc1ccccc1)n1cnc2ncccc21] | 1.0 | 26.6 | A |
| 63 | 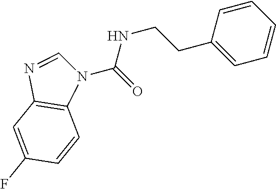 [SMILES: O=C(NCCc1ccccc1)n1cnc2cc(F)ccc21] | 2.3 | 2.6 | A |
| 64 | 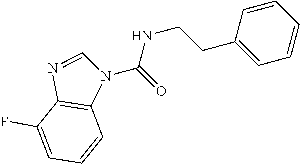 [SMILES: O=C(NCCc1ccccc1)n1cnc2c(F)cccc21] | 2.3 | 3.6 | A |
| 65 | 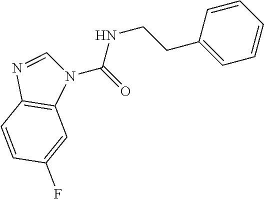 [SMILES: O=C(NCCc1ccccc1)n1cnc2ccc(F)cc21] | 2.3 | 15.1 | A |
| 66b | 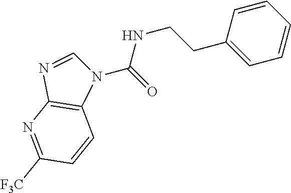 [SMILES: O=C(NCCc1ccccc1)n1cnc2nc(C(F)(F)F)ccc21] | 1.9 | 12.9 | A |
| 67 | 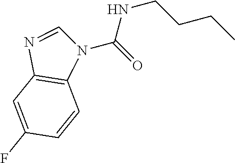 [SMILES: CCCCNC(=O)n1cnc2cc(F)ccc21] | 1.8 | 22.5 | A |
| 68 | 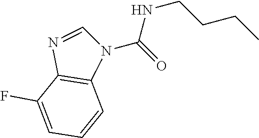 [SMILES: CCCCNC(=O)n1cnc2c(F)cccc21] | 1.8 | 20.6 | A |
| 69 | 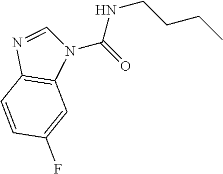 [SMILES: CCCCNC(=O)n1cnc2ccc(F)cc21] | 1.8 | 20.2 | B |
| 70a | 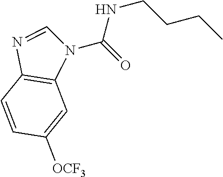 [SMILES: CCCCNC(=O)n1cnc2ccc(OC(F)(F)F)cc21] | 3.0 | 9.3 | A |
| 70b | 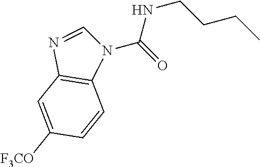 [SMILES: CCCCNC(=O)n1cnc2cc(OC(F)(F)F)ccc21] | 3.0 | 9.6 | A |
| 71 | 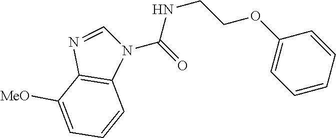 [SMILES: COc1cccc2c1ncn2C(=O)NCCOc1ccccc1] | 2.5 | 9.1 | A |
| 72 | 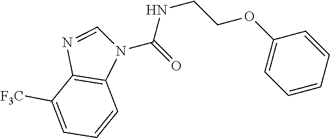 [SMILES: O=C(NCCOc1ccccc1)n1cnc2c(C(F)(F)F)cccc21] | 3.1 | 2.0 | A |
| 73 | 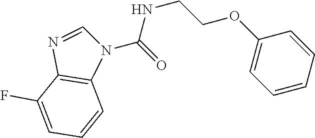 [SMILES: O=C(NCCOc1ccccc1)n1cnc2c(F)cccc21] | 2.4 | 2.7 | A |
| 74 | 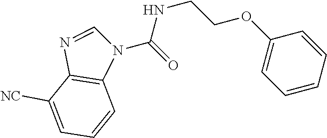 [SMILES: N#Cc1cccc2c1ncn2C(=O)NCCOc1ccccc1] | 1.7 | 5.9 | A |
| 75 | 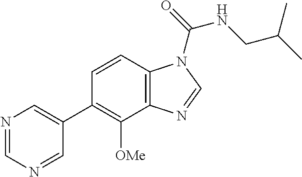 [SMILES: COc1c(-c2cncnc2)ccc2c1ncn2C(=O)NCC(C)C] | 0.6 | 27.0 | B |
| 76 | 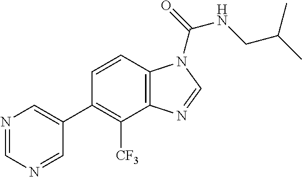 [SMILES: CC(C)CNC(=O)n1cnc2c(C(F)(F)F)c(-c3cncnc3)ccc21] | 1.9 | 10.0 | A |
| 77a | 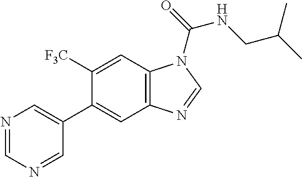 [SMILES: CC(C)CNC(=O)n1cnc2cc(-c3cncnc3)c(C(F)(F)F)cc21] | 1.9 | 14.5 | A |
| 77b | 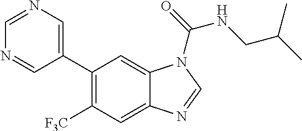 [SMILES: CC(C)CNC(=O)n1cnc2cc(C(F)(F)F)c(-c3cncnc3)cc21] | 1.9 | 7.4 | A |
| 79a | 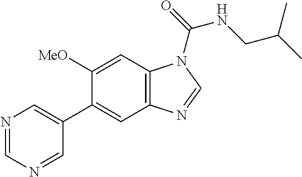 [SMILES: COc1cc2c(cc1-c1cncnc1)ncn2C(=O)NCC(C)C] | 0.6 | 22.2 | B |
| 79b | 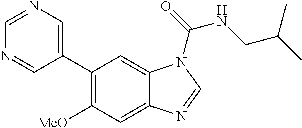 [SMILES: COc1cc2ncn(C(=O)NCC(C)C)c2cc1-c1cncnc1] | 0.6 | 26.5 | A |
| 78 | 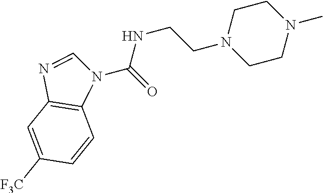 [SMILES: CN1CCN(CCNC(=O)n2cnc3cc(C(F)(F)F)ccc32)CC1] | 0.6 | 32.7 | C |
| 80 | 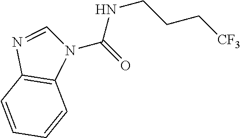 [SMILES: O=C(NCCCC(F)(F)F)n1cnc2ccccc21] | 1.2 | 21.1 | A |
| 81a | 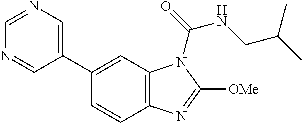 [SMILES: COc1nc2ccc(-c3cncnc3)cc2n1C(=O)NCC(C)C] | 1.7 | 22.8 | B |
| 81b | 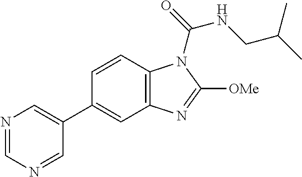 [SMILES: COc1nc2cc(-c3cncnc3)ccc2n1C(=O)NCC(C)C] | 1.7 | 27 | B |
| 82a | 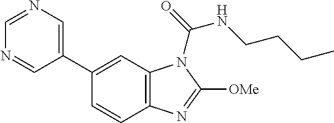 [SMILES: CCCCNC(=O)n1c(OC)nc2ccc(-c3cncnc3)cc21] | 1.8 | 21.8 | B |
| 82b | 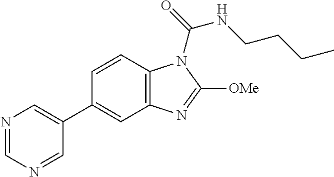 [SMILES: CCCCNC(=O)n1c(OC)nc2cc(-c3cncnc3)ccc21] | 1.8 | 24.5 | B |
| 83 | 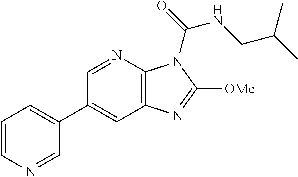 [SMILES: COc1nc2cc(-c3cccnc3)cnc2n1C(=O)NCC(C)C] | 1.7 | 16.9 | B |
| 85a | 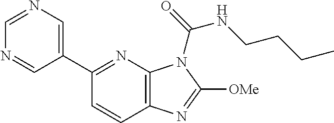 [SMILES: CCCCNC(=O)n1c(OC)nc2ccc(-c3cncnc3)nc21] | 1.0 | 40 | A |
| 84 | 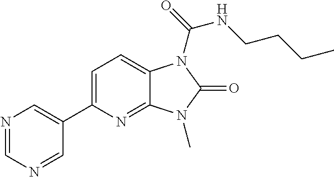 [SMILES: CCCCNC(=O)n1c(=O)n(C)c2nc(-c3cncnc3)ccc21] | 1.2 | 0.9 | B |
| 85b | 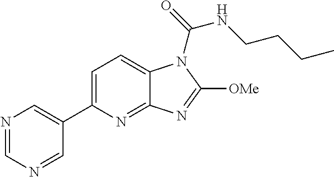 [SMILES: CCCCNC(=O)n1c(OC)nc2nc(-c3cncnc3)ccc21] | 1.0 | 54 | A |
| 86 | 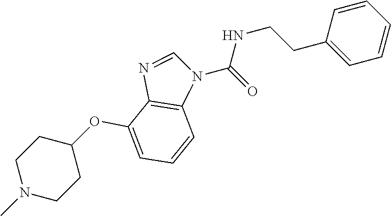 [SMILES: CN1CCC(Oc2cccc3c2ncn3C(=O)NCCc2ccccc2)CC1] | 2.4 | 26.5 | A |
| 87 | 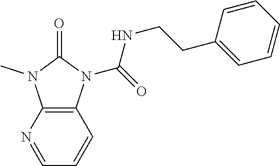 [SMILES: Cn1c(=O)n(C(=O)NCCc2ccccc2)c2cccnc21] | 2.2 | 14.7 | C |
| 88 | 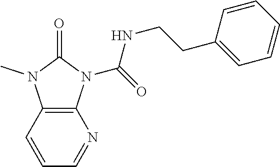 [SMILES: Cn1c(=O)n(C(=O)NCCc2ccccc2)c2ncccc21] | 2.2 | 26.4 | A |
| 89 | 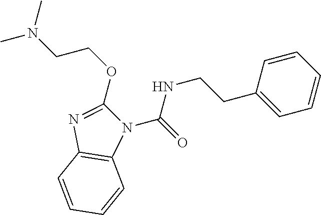 [SMILES: CN(C)CCOc1nc2ccccc2n1C(=O)NCCc1ccccc1] | 3.1 | 27.6 | A |
| 90 | 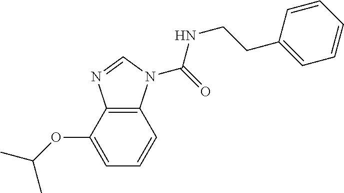 [SMILES: CC(C)Oc1cccc2c1ncn2C(=O)NCCc1ccccc1] | 3.2 | 0.3 | A |
| 91a | 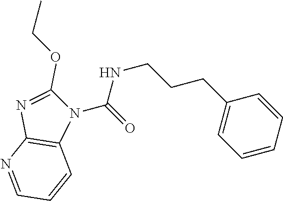 [SMILES: CCOc1nc2ncccc2n1C(=O)NCCCc1ccccc1] | 2.8 | 7.0 | A |
| 91b | 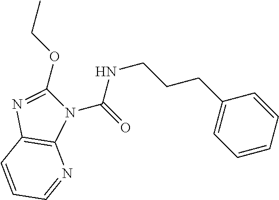 [SMILES: CCOc1nc2cccnc2n1C(=O)NCCCc1ccccc1] | 2.8 | 21.1 | A |
| 92 | 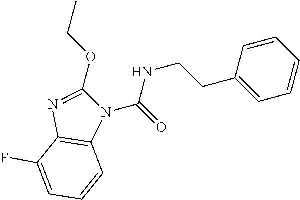 [SMILES: CCOc1nc2c(F)cccc2n1C(=O)NCCc1ccccc1] | 3.6 | 1.2 | A |
| 93 | 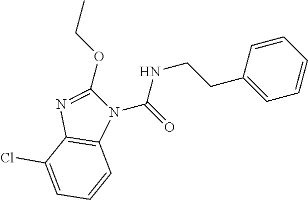 [SMILES: CCOc1nc2c(Cl)cccc2n1C(=O)NCCc1ccccc1] | 4.2 | 0.05 | A |
| 94 | 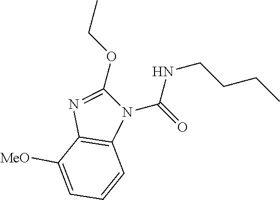 [SMILES: CCCCNC(=O)n1c(OCC)nc2c(OC)cccc21] | 3.5 | 11.2 | B |
| 95 | 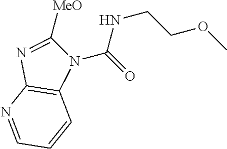 [SMILES: COCCNC(=O)n1c(OC)nc2ncccc21] | 0.1 | NA | B |
| 96 | 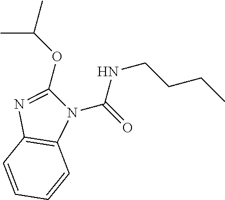 [SMILES: CCCCNC(=O)n1c(OC(C)C)nc2ccccc21] | 3.2 | 10.3 | C |
| 97 | 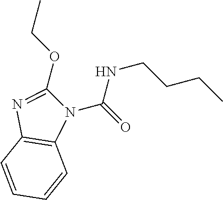 [SMILES: CCCCNC(=O)n1c(OCC)nc2ccccc21] | 2.9 | 10.9 | B |
| 97a | 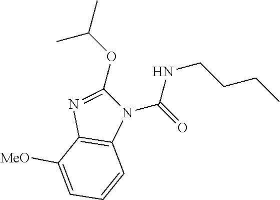 [SMILES: CCCCNC(=O)n1c(OC(C)C)nc2c(OC)cccc21] | 3.5 | 18.0 | B |
| 98 | 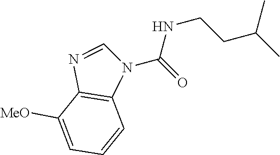 [SMILES: COc1cccc2c1ncn2C(=O)NCCC(C)C] | 2.3 | 16.0 | A |
| 100 | 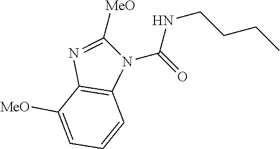 [SMILES: CCCCNC(=O)n1c(OC)nc2c(OC)cccc21] | 2.7 | 28.0 | B |
| 101a | 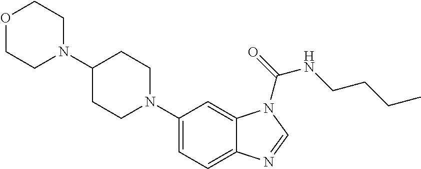 [SMILES: CCCCNC(=O)n1cnc2ccc(N3CCC(N4CCOCC4)CC3)cc21] | 1.6 | 26.7 | A |
| 101b | 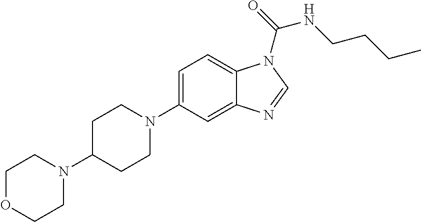 [SMILES: CCCCNC(=O)n1cnc2cc(N3CCC(N4CCOCC4)CC3)ccc21] | 1.6 | 7.5 | A |
| 102a | 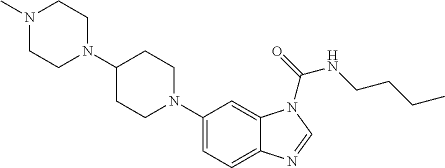 [SMILES: CCCCNC(=O)n1cnc2ccc(N3CCC(N4CCN(C)CC4)CC3)cc21] | 0.7 | 34.8 | A |
| 102b | 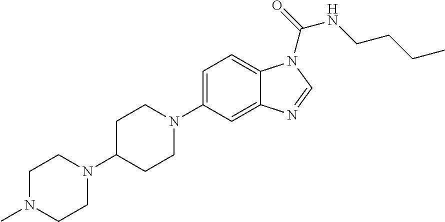 [SMILES: CCCCNC(=O)n1cnc2cc(N3CCC(N4CCN(C)CC4)CC3)ccc21] | 0.7 | 36.1 | B |
| 103a | 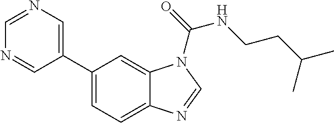 [SMILES: CC(C)CCNC(=O)n1cnc2ccc(-c3cncnc3)cc21] | 1.5 | 14.3 | A |
| 103b | 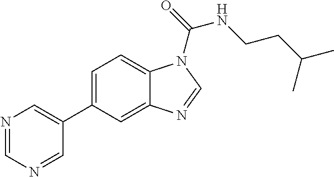 [SMILES: CC(C)CCNC(=O)n1cnc2cc(-c3cncnc3)ccc21] | 1.5 | 11.9 | A |
| 104 | 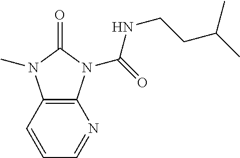 [SMILES: CC(C)CCNC(=O)n1c(=O)n(C)c2cccnc21] | 2.1 | 17 | B |
| 105 | 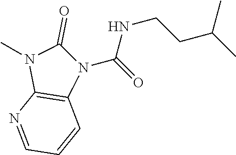 [SMILES: CC(C)CCNC(=O)n1c(=O)n(C)c2ncccc21] | 2.1 | 17.4 | C |
| 106 | 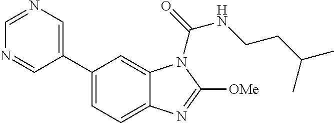 [SMILES: COc1nc2ccc(-c3cncnc3)cc2n1C(=O)NCCC(C)C] | 2.2 | 13.7 | B |
| 107 | 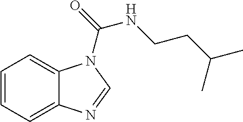 [SMILES: CC(C)CCNC(=O)n1cnc2ccccc21] | 2.0 | 13.9 | A |
| 107a | 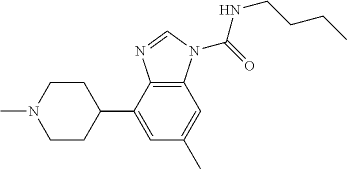 [SMILES: CCCCNC(=O)n1cnc2c(C3CCN(C)CC3)cc(C)cc21] | 2.6 | 31.7 | B |
| 108 | 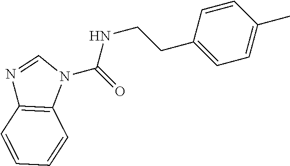 [SMILES: Cc1ccc(CCNC(=O)n2cnc3ccccc32)cc1] | 2.6 | 6.8 | A |
| 109a | 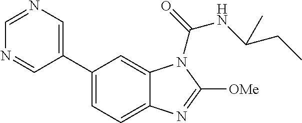 [SMILES: CCC(C)NC(=O)n1c(OC)nc2ccc(-c3cncnc3)cc21] | 1.6 | 28.7 | C |
| 109b | 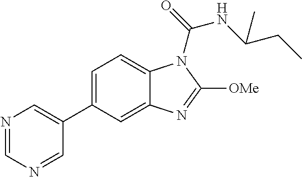 [SMILES: CCC(C)NC(=O)n1c(OC)nc2cc(-c3cncnc3)ccc21] | 1.6 | 32.4 | C |
| 110 | 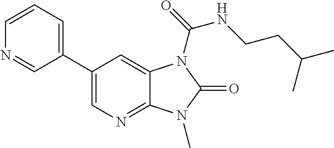 [SMILES: CC(C)CCNC(=O)n1c(=O)n(C)c2ncc(-c3cccnc3)cc21] | 2.6 | 18 | C |
| 111a | 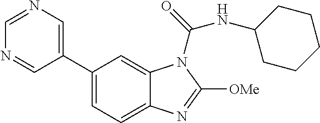 [SMILES: COc1nc2ccc(-c3cncnc3)cc2n1C(=O)NC1CCCCC1] | 2.3 | 6 | A |
| 111b | 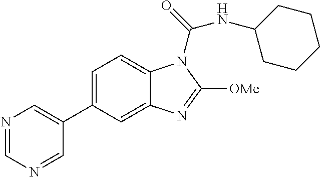 [SMILES: COc1nc2cc(-c3cncnc3)ccc2n1C(=O)NC1CCCCC1] | 2.3 | 22 | A |
| 112a | 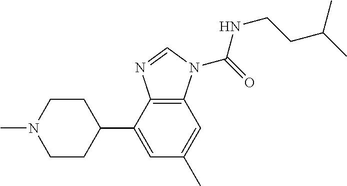 [SMILES: Cc1cc(C2CCN(C)CC2)c2ncn(C(=O)NCCC(C)C)c2c1] | 3.0 | 22.9 | B |
| 112b | 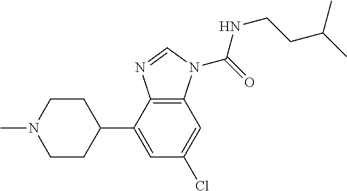 [SMILES: CC(C)CCNC(=O)n1cnc2c(C3CCN(C)CC3)cc(Cl)cc21] | 3.2 | 20.0 | B |
| 112 | 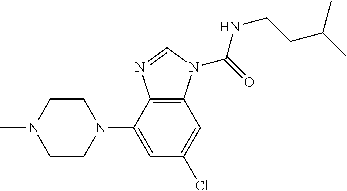 [SMILES: CC(C)CCNC(=O)n1cnc2c(N3CCN(C)CC3)cc(Cl)cc21] | 3.2 | 27.5 | NA |
| 113 | 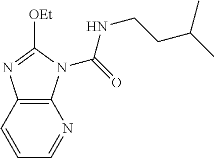 [SMILES: CCOc1nc2cccnc2n1C(=O)NCCC(C)C] | 2.3 | N/A | A |
| 114 | 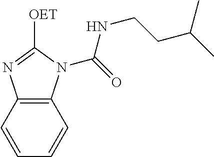 | 2.3 | 16.8 | B |
| 115 | 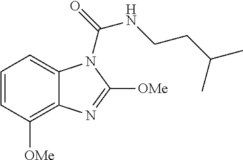 [SMILES: COc1cccc2c1nc(OC)n2C(=O)NCCC(C)C] | 3.1 | 15.4 | C |
| 116 | 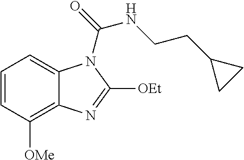 [SMILES: CCOc1nc2c(OC)cccc2n1C(=O)NCCC1CC1] | 3.1 | 14.1 | C |
| 117 | 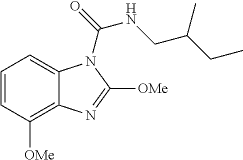 [SMILES: CCC(C)CNC(=O)n1c(OC)nc2c(OC)cccc21] | 3.1 | 23.4 | B |
| 118 | 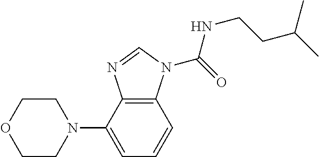 [SMILES: CC(C)CCNC(=O)n1cnc2c(N3CCOCC3)cccc21] | 1.9 | 21 | B |
| 119 | 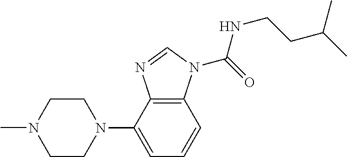 [SMILES: CC(C)CCNC(=O)n1cnc2c(N3CCN(C)CC3)cccc21] | 2.3 | 23 | B |
| 120 | 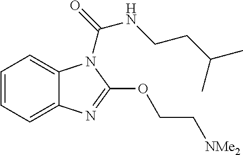 [SMILES: CC(C)CCNC(=O)n1c(OCCN(C)C)nc2ccccc21] | 2.9 | 21 | A |
| 121 | 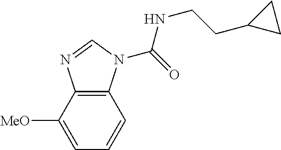 [SMILES: COc1cccc2c1ncn2C(=O)NCCC1CC1] | 1.8 | 22.6 | B |
| 122 | 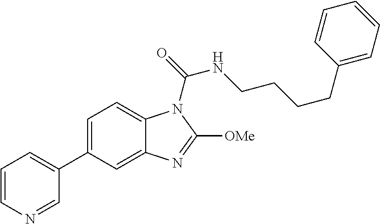 [SMILES: COc1nc2cc(-c3cccnc3)ccc2n1C(=O)NCCCCc1ccccc1] | 0.8 | 0.04 | A |
| 123 | 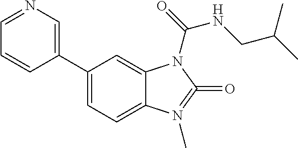 | 3.5 | 0.6 | C |
| 124 | 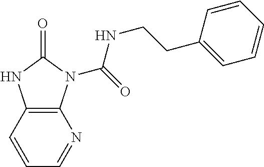 [SMILES: O=C(NCCc1ccccc1)n1c(=O)[nH]c2cccnc21] | 1.6 | 4 | C |
| 125 | 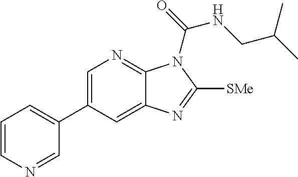 [SMILES: CSc1nc2cc(-c3cccnc3)cnc2n1C(=O)NCC(C)C] | 2.0 | 6 | B |
| 126a | 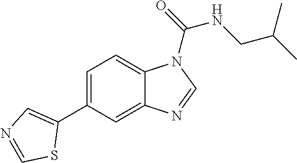 [SMILES: CC(C)CNC(=O)n1cnc2cc(-c3cncs3)ccc21] | 2.0 | 6 | A |
| 126b | 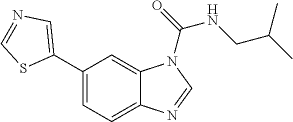 [SMILES: CC(C)CNC(=O)n1cnc2ccc(-c3cncs3)cc21] | 2.0 | 14 | B |
| 127 | 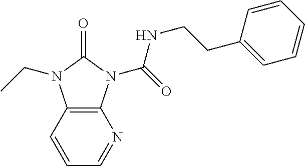 [SMILES: CCn1c(=O)n(C(=O)NCCc2ccccc2)c2ncccc21] | 2.7 | 23 | B |
| 128 | 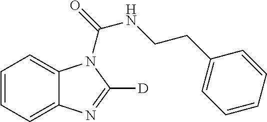 [SMILES: [2H]c1nc2ccccc2n1C(=O)NCCc1ccccc1] | 2.1 | 15 | A |
| 129 | 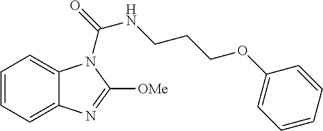 [SMILES: COc1nc2ccccc2n1C(=O)NCCCOc1ccccc1] | 2.3 | 1.0 | A |
| 130 | 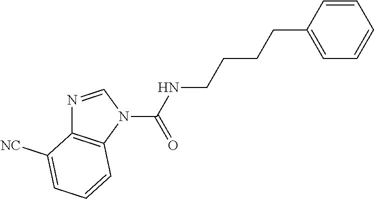 [SMILES: N#Cc1cccc2c1ncn2C(=O)NCCCCc1ccccc1] | 2.5 | 1.3 | A |
| 131 | 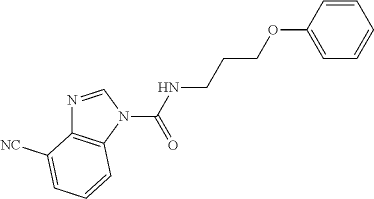 [SMILES: N#Cc1cccc2c1ncn2C(=O)NCCCOc1ccccc1] | 2.1 | 5 | A |
| 132 | 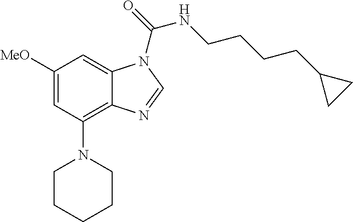 [SMILES: COc1cc(N2CCCCC2)c2ncn(C(=O)NCCCCC3CC3)c2c1] | 4.1 | 0.2 | A |
| 133a | 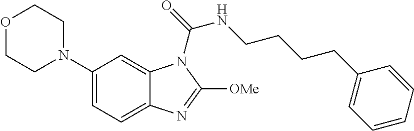 [SMILES: COc1nc2ccc(N3CCOCC3)cc2n1C(=O)NCCCCc1ccccc1] | 3.7 | 1 | A |
| 133b | 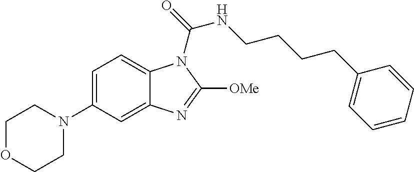 [SMILES: COc1nc2cc(N3CCOCC3)ccc2n1C(=O)NCCCCc1ccccc1] | 3.7 | 2 | A |
| 134a | 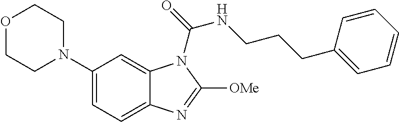 [SMILES: COc1nc2ccc(N3CCOCC3)cc2n1C(=O)NCCCc1ccccc1] | 3.2 | 17 | A |
| 134b | 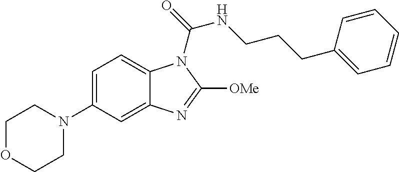 [SMILES: COc1nc2cc(N3CCOCC3)ccc2n1C(=O)NCCCc1ccccc1] | 3.2 | 16 | A |
| 135a | 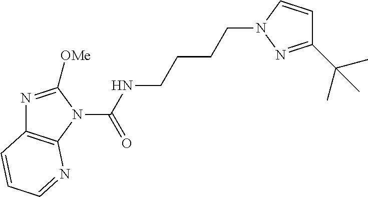 [SMILES: COc1nc2cccnc2n1C(=O)NCCCCn1ccc(C(C)(C)C)n1] | 2.0 | 15 | A |
| 135b | 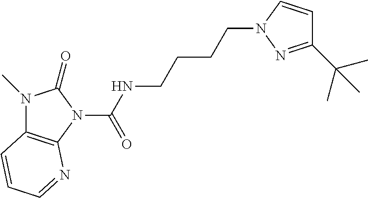 [SMILES: Cn1c(=O)n(C(=O)NCCCCn2ccc(C(C)(C)C)n2)c2ncccc21] | 2.3 | 23 | NA |
| 136 | 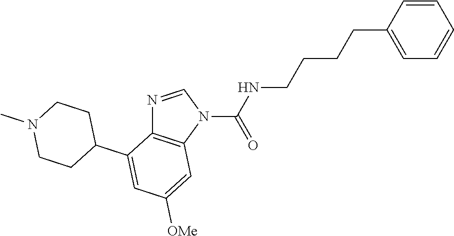 [SMILES: COc1cc(C2CCN(C)CC2)c2ncn(C(=O)NCCCCc3ccccc3)c2c1] | 3.8 | 21 | A |
| 137 | 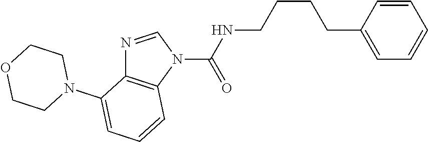 [SMILES: O=C(NCCCCc1ccccc1)n1cnc2c(N3CCOCC3)cccc21] | 2.9 | 0.6 | A |
| 138 | 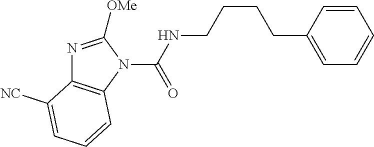 [SMILES: COc1nc2c(C#N)cccc2n1C(=O)NCCCCc1ccccc1] | 3.3 | 0.3 | A |
| 139 | 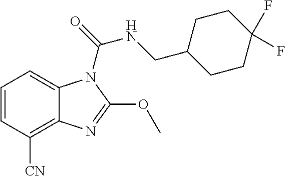 [SMILES: COc1nc2c(C#N)cccc2n1C(=O)NCC1CCC(F)(F)CC1] | 2.3 | 9 | A |
| 140 | 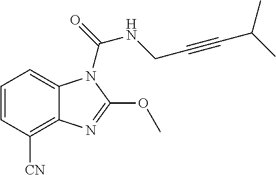 [SMILES: COc1nc2c(C#N)cccc2n1C(=O)NCC#CC(C)C] | 1.8 | 10 | A |
| 141 | 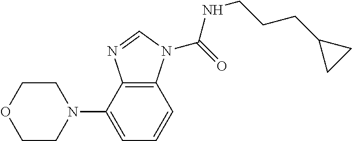 [SMILES: O=C(NCCCC1CC1)n1cnc2c(N3CCOCC3)cccc21] | 1.9 | 15 | A |
| 142 | 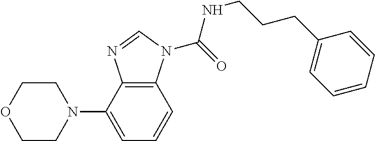 [SMILES: O=C(NCCCc1ccccc1)n1cnc2c(N3CCOCC3)cccc21] | 2.4 | 1.4 | A |
| 143 | 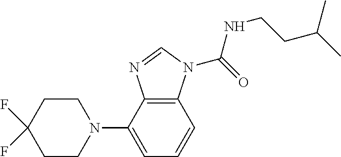 [SMILES: CC(C)CCNC(=O)n1cnc2c(N3CCC(F)(F)CC3)cccc21] | 2.6 | 2 | B |
| 144 | 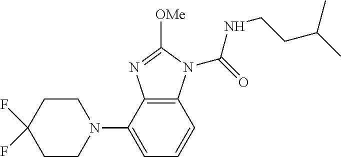 [SMILES: COc1nc2c(N3CCC(F)(F)CC3)cccc2n1C(=O)NCCC(C)C] | 3.4 | 1.2 | C |
| 145 | 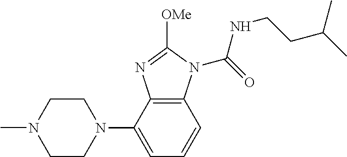 [SMILES: COc1nc2c(N3CCN(C)CC3)cccc2n1C(=O)NCCC(C)C] | 3.1 | 46 | C |
| 146 | 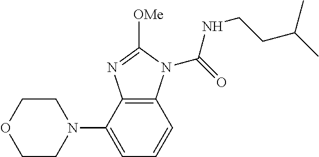 [SMILES: COc1nc2c(N3CCOCC3)cccc2n1C(=O)NCCC(C)C] | 2.7 | 17 | C |
| 147 | 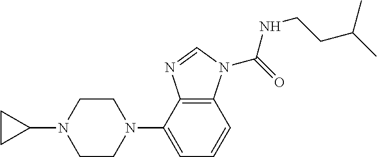 [SMILES: CC(C)CCNC(=O)n1cnc2c(N3CCN(C4CC4)CC3)cccc21] | 2.9 | 14 | B |
| 148 | 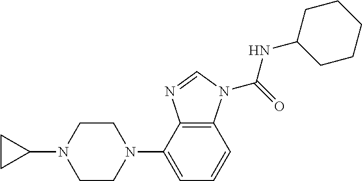 [SMILES: O=C(NC1CCCCC1)n1cnc2c(N3CCN(C4CC4)CC3)cccc21] | 2.9 | 3 | C |
| 149 | 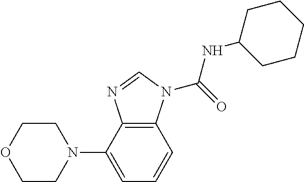 [SMILES: O=C(NC1CCCCC1)n1cnc2c(N3CCOCC3)cccc21] | 1.9 | 15 | C |
| 150 | 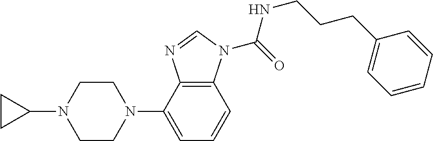 [SMILES: O=C(NCCCc1ccccc1)n1cnc2c(N3CCN(C4CC4)CC3)cccc21] | 3.3 | 1.5 | A |
| 151 | 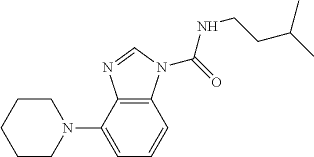 [SMILES: CC(C)CCNC(=O)n1cnc2c(N3CCCCC3)cccc21] | 3.3 | 2 | B |
| 152 | 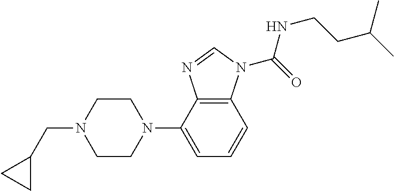 [SMILES: CC(C)CCNC(=O)n1cnc2c(N3CCN(CC4CC4)CC3)cccc21] | 3.3 | 20 | B |
| 153 | 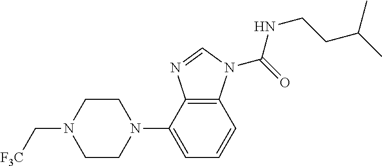 [SMILES: CC(C)CCNC(=O)n1cnc2c(N3CCN(CC(F)(F)F)CC3)cccc21] | 3.1 | 10 | B |
| 154 | 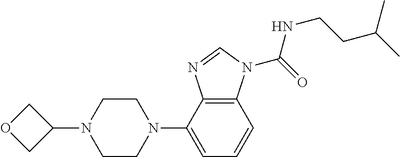 [SMILES: CC(C)CCNC(=O)n1cnc2c(N3CCN(C4COC4)CC3)cccc21] | 2.0 | 30 | B |
| 155 | 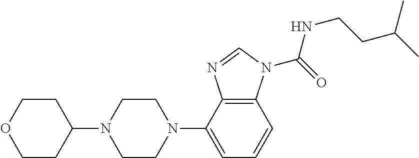 [SMILES: CC(C)CCNC(=O)n1cnc2c(N3CCN(C4CCOCC4)CC3)cccc21] | 1.9 | 30 | B |
| 156 | 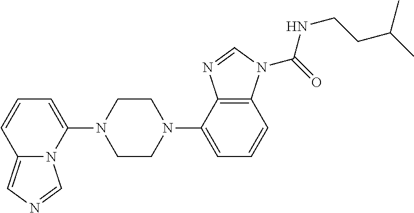 [SMILES: CC(C)CCNC(=O)n1cnc2c(N3CCN(c4cccc5cncn45)CC3)cccc21] | 3.5 | 0.2 | B |
| 157 | 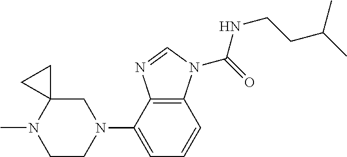 [SMILES: CC(C)CCNC(=O)n1cnc2c(N3CCN(C)C4(CC4)C3)cccc21] | 2.7 | 27 | B |
| 158 | 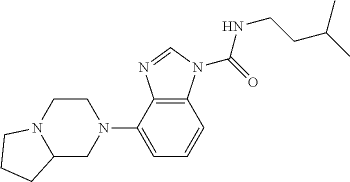 [SMILES: CC(C)CCNC(=O)n1cnc2c(N3CCN4CCCC4C3)cccc21] | 2.7 | 17 | B |
| 159 | 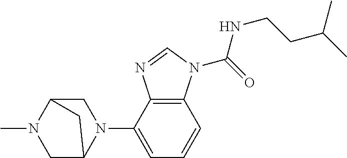 [SMILES: CC(C)CCNC(=O)n1cnc2c(N3CC4CC3CN4C)cccc21] | 2.5 | 33 | B |
| 160 | 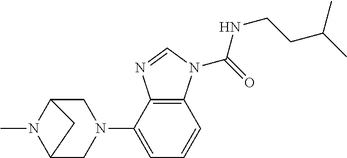 [SMILES: CC(C)CCNC(=O)n1cnc2c(N3CC4CC(C3)N4C)cccc21] | 2.1 | 35 | B |
| 161 | 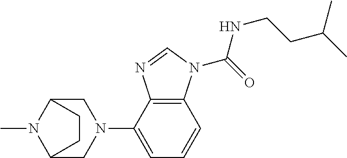 [SMILES: CC(C)CCNC(=O)n1cnc2c(N3CC4CCC(C3)N4C)cccc21] | 2.6 | 35 | B |
| 162 | 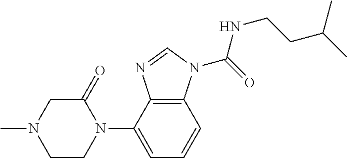 [SMILES: CC(C)CCNC(=O)n1cnc2c(N3CCN(C)CC3=O)cccc21] | 2.2 | 18 | C |
| 163 | 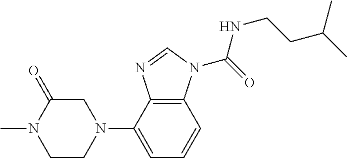 [SMILES: CC(C)CCNC(=O)n1cnc2c(N3CCN(C)C(=O)C3)cccc21] | 2.0 | 14 | B |
| 164 | 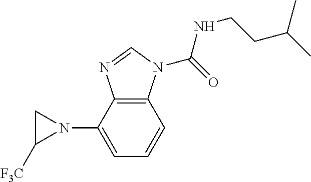 [SMILES: CC(C)CCNC(=O)n1cnc2c(N3CC3C(F)(F)F)cccc21] | 2.6 | 2 | A |
| 165 | 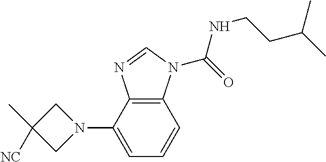 [SMILES: CC(C)CCNC(=O)n1cnc2c(N3CC(C)(C#N)C3)cccc21] | 2.3 | 1 | B |
| 166 | 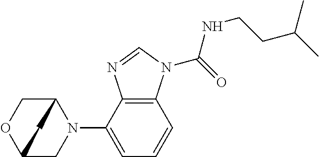 | 2.1 | 12 | B |
| 167 | 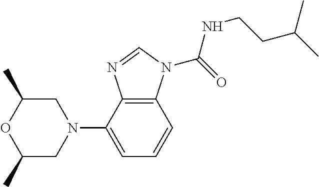 [SMILES: CC(C)CCNC(=O)n1cnc2c(N3C[C@@H](C)O[C@@H](C)C3)cccc21] | 2.9 | 4 | B |
| 168 | 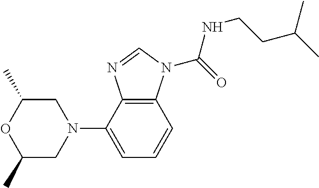 [SMILES: CC(C)CCNC(=O)n1cnc2c(N3C[C@@H](C)O[C@H](C)C3)cccc21] | 2.9 | 11 | B |
| 169 | 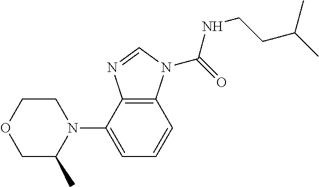 [SMILES: CC(C)CCNC(=O)n1cnc2c(N3CCOC[C@@H]3C)cccc21] | 2.4 | 23 | B |
| 170 | 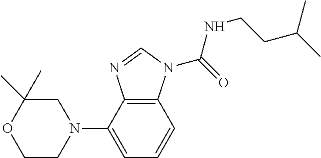 [SMILES: CC(C)CCNC(=O)n1cnc2c(N3CCOC(C)(C)C3)cccc21] | 2.9 | 14 | B |
| 171 | 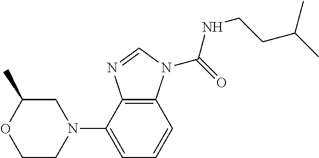 [SMILES: CC(C)CCNC(=O)n1cnc2c(N3CCO[C@@H](C)C3)cccc21] | 2.4 | 15 | B |
| 172a | 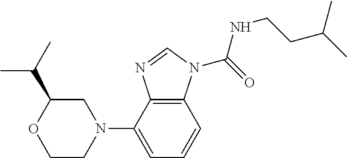 [SMILES: CC(C)CCNC(=O)n1cnc2c(N3CCO[C@@H](C(C)C)C3)cccc21] | 3.3 | 4 | B |
| 172b | 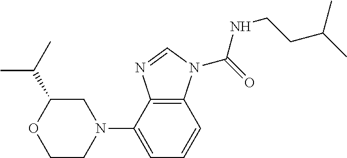 [SMILES: CC(C)CCNC(=O)n1cnc2c(N3CCO[C@H](C(C)C)C3)cccc21] | 3.3 | 4 | B |
| 173 | 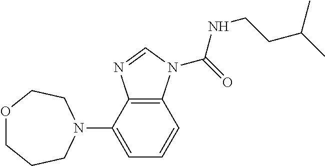 [SMILES: CC(C)CCNC(=O)n1cnc2c(N3CCCOCC3)cccc21] | 1.9 | 17 | B |
| 174 | 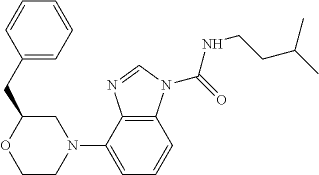 [SMILES: CC(C)CCNC(=O)n1cnc2c(N3CCO[C@@H](Cc4ccccc4)C3)cccc21] | 4.0 | 1 | B |
| 175 | 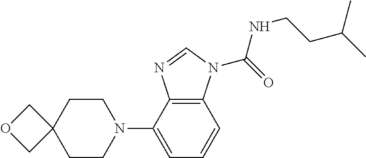 [SMILES: CC(C)CCNC(=O)n1cnc2c(N3CCC4(CC3)COC4)cccc21] | 1.7 | 18 | B |
| 176 | 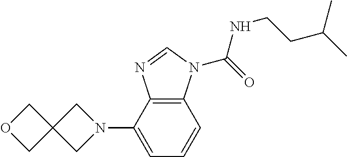 [SMILES: CC(C)CCNC(=O)n1cnc2c(N3CC4(COC4)C3)cccc21] | 2.0 | 22 | B |
| 177 | 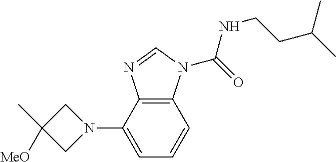 [SMILES: COC1(C)CN(c2cccc3c2ncn3C(=O)NCCC(C)C)C1] | 2.9 | 13 | B |
| 178 | 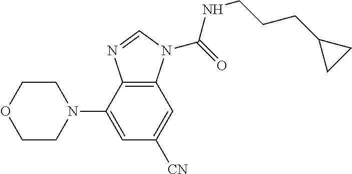 [SMILES: N#Cc1cc(N2CCOCC2)c2ncn(C(=O)NCCCC3CC3)c2c1] | 1.5 | 2 | A |
| 179 | 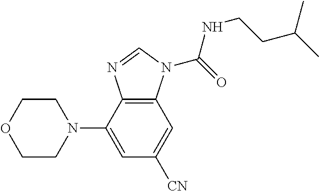 [SMILES: CC(C)CCNC(=O)n1cnc2c(N3CCOCC3)cc(C#N)cc21] | 1.4 | 10 | A |
| 180 | 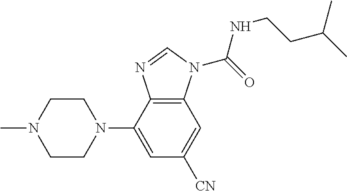 [SMILES: CC(C)CCNC(=O)n1cnc2c(N3CCN(C)CC3)cc(C#N)cc21] | 1.9 | 23 | A |
| 181a | 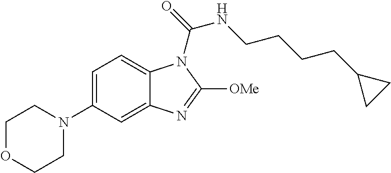 [SMILES: COc1nc2cc(N3CCOCC3)ccc2n1C(=O)NCCCCC1CC1] | 3.2 | 19 | B |
| 181b | 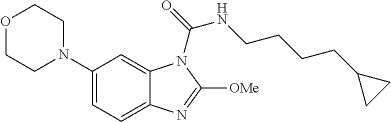 [SMILES: COc1nc2ccc(N3CCOCC3)cc2n1C(=O)NCCCCC1CC1] | 3.2 | 11 | A |
| 182 | 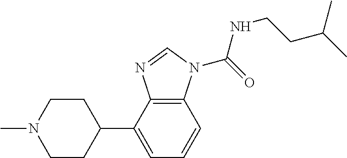 [SMILES: CC(C)CCNC(=O)n1cnc2c(C3CCN(C)CC3)cccc21] | 2.5 | 32 | B |
| 183a | 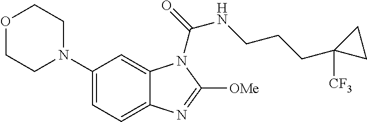 [SMILES: COc1nc2ccc(N3CCOCC3)cc2n1C(=O)NCCCC1(C(F)(F)F)CC1] | 2.5 | 15 | A |
| 183b | 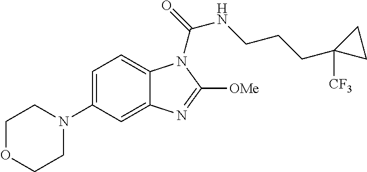 [SMILES: COc1nc2cc(N3CCOCC3)ccc2n1C(=O)NCCCC1(C(F)(F)F)CC1] | 2.5 | 3 | A |
| 184a | 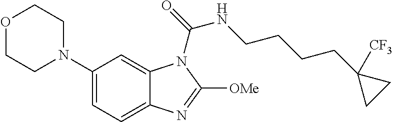 [SMILES: COc1nc2ccc(N3CCOCC3)cc2n1C(=O)NCCCCC1(C(F)(F)F)CC1] | 3.0 | 3 | A |
| 184b | 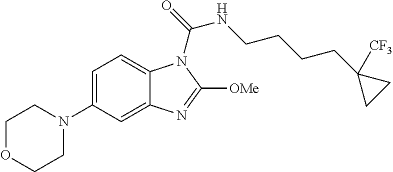 [SMILES: COc1nc2cc(N3CCOCC3)ccc2n1C(=O)NCCCCC1(C(F)(F)F)CC1] | 3.0 | 3 | B |
| 185 | 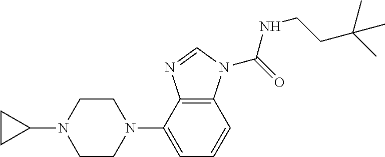 [SMILES: CC(C)(C)CCNC(=O)n1cnc2c(N3CCN(C4CC4)CC3)cccc21] | 3.2 | 2 | B |
| 186 | 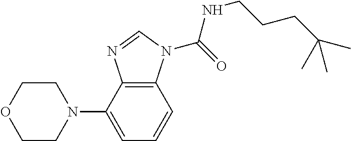 [SMILES: CC(C)(C)CCCNC(=O)n1cnc2c(N3CCOCC3)cccc21] | 2.8 | 10 | A |
| 187 | 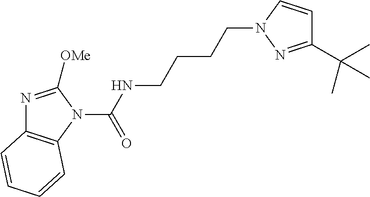 [SMILES: COc1nc2ccccc2n1C(=O)NCCCCn1ccc(C(C)(C)C)n1] | 3.0 | 22 | B |
| 188a | 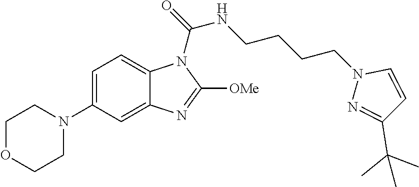 [SMILES: COc1nc2cc(N3CCOCC3)ccc2n1C(=O)NCCCCn1ccc(C(C)(C)C)n1] | 2.9 | 27 | B |
| 188b | 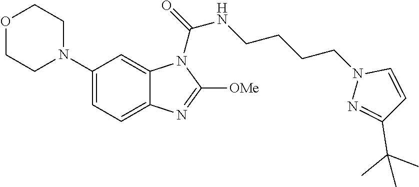 [SMILES: COc1nc2ccc(N3CCOCC3)cc2n1C(=O)NCCCCn1ccc(C(C)(C)C)n1] | 2.9 | 26 | B |
| 189a | 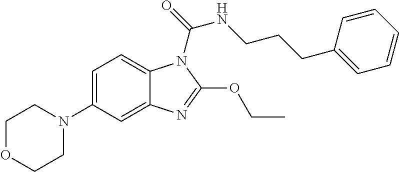 [SMILES: CCOc1nc2cc(N3CCOCC3)ccc2n1C(=O)NCCCc1ccccc1] | 3.7 | 2 | A |
| 189b | 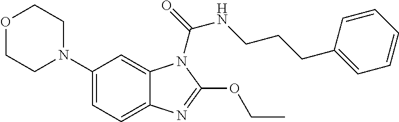 [SMILES: CCOc1nc2ccc(N3CCOCC3)cc2n1C(=O)NCCCc1ccccc1] | 3.7 | 3 | A |
| 190a | 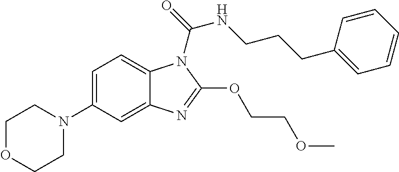 [SMILES: COCCOc1nc2cc(N3CCOCC3)ccc2n1C(=O)NCCCc1ccccc1] | 3.0 | 17 | A |
| 191 | 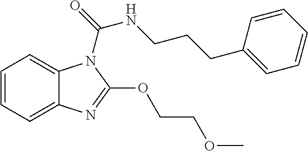 [SMILES: COCCOc1nc2ccccc2n1C(=O)NCCCc1ccccc1] | 3.2 | 4 | A |
| 190b | 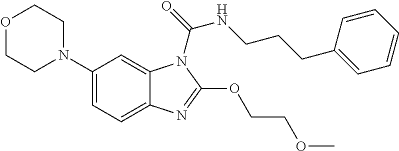 [SMILES: COCCOc1nc2ccc(N3CCOCC3)cc2n1C(=O)NCCCc1ccccc1] | 3.0 | 16 | A |
| 192 | 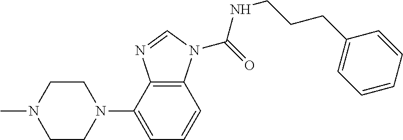 [SMILES: CN1CCN(c2cccc3c2ncn3C(=O)NCCCc2ccccc2)CC1] | 2.9 | 12 | A |
| 193 | 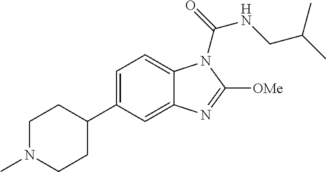 [SMILES: COc1nc2cc(C3CCN(C)CC3)ccc2n1C(=O)NCC(C)C] | 2.8 | 39 | C |
| 194 | 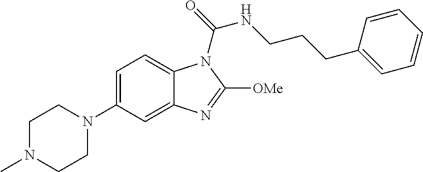 [SMILES: COc1nc2cc(N3CCN(C)CC3)ccc2n1C(=O)NCCCc1ccccc1] | 3.6 | 30 | B |
| 195a | 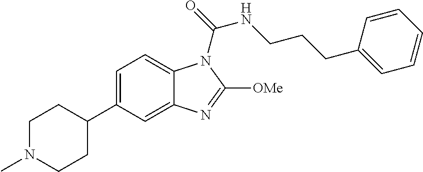 [SMILES: COc1nc2cc(C3CCN(C)CC3)ccc2n1C(=O)NCCCc1ccccc1] | 3.8 | 43 | C |
| 195b | 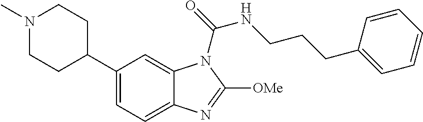 [SMILES: COc1nc2ccc(C3CCN(C)CC3)cc2n1C(=O)NCCCc1ccccc1] | 3.8 | 39 | C |
| 196 | 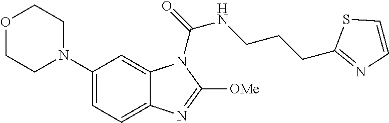 [SMILES: COc1nc2ccc(N3CCOCC3)cc2n1C(=O)NCCCc1nccs1] | 1.5 | 35 | B |
| 197 | 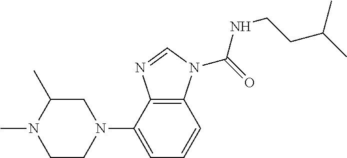 [SMILES: CC(C)CCNC(=O)n1cnc2c(N3CCN(C)C(C)C3)cccc21] | 2.8 | 28 | B |
| 198 | 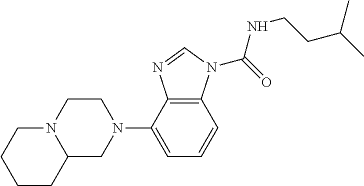 [SMILES: CC(C)CCNC(=O)n1cnc2c(N3CCN4CCCCC4C3)cccc21] | 3.2 | 28 | B |
| 199 | 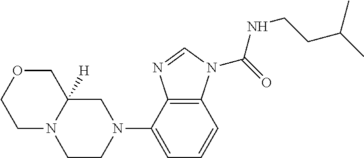 [SMILES: CC(C)CCNC(=O)n1cnc2c(N3CCN4CCOC[C@H]4C3)cccc21] | 2.1 | 31 | C |
| 200 | 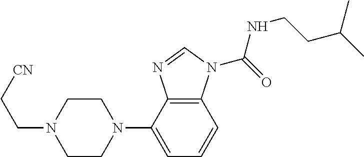 [SMILES: CC(C)CCNC(=O)n1cnc2c(N3CCN(CCC#N)CC3)cccc21] | 2.1 | 30 | B |
| 201 | 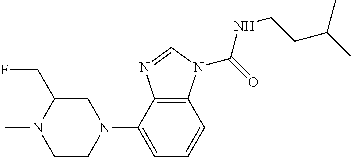 [SMILES: CC(C)CCNC(=O)n1cnc2c(N3CCN(C)C(CF)C3)cccc21] | 2 7 | 28 | B |
| 202 | 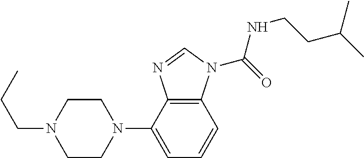 [SMILES: CCCN1CCN(c2cccc3c2ncn3C(=O)NCCC(C)C)CC1] | 3.5 | 15 | B |
| 203 | 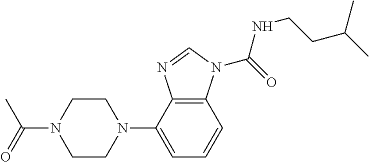 [SMILES: CC(=O)N1CCN(c2cccc3c2ncn3C(=O)NCCC(C)C)CC1] | 1.5 | 26 | A |
| 204 | 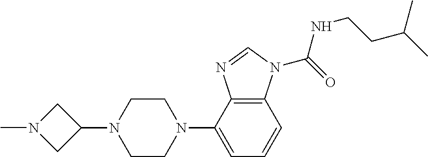 [SMILES: CC(C)CCNC(=O)n1cnc2c(N3CCN(C4CN(C)C4)CC3)cccc21] | 1.0 | 36 | B |
| 205 | 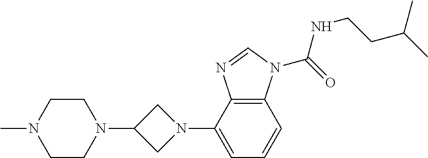 [SMILES: CC(C)CCNC(=O)n1cnc2c(N3CC(N4CCN(C)CC4)C3)cccc21] | 1.1 | 31 | B |
| 206 | 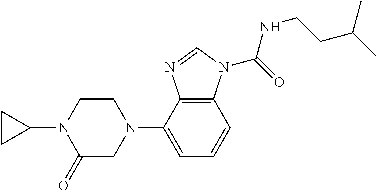 [SMILES: CC(C)CCNC(=O)n1cnc2c(N3CCN(C4CC4)C(=O)C3)cccc21] | 2.5 | 20 | B |
| 207 | 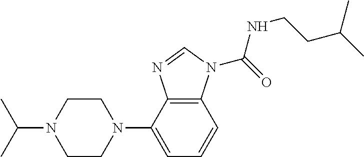 [SMILES: CC(C)CCNC(=O)n1cnc2c(N3CCN(C(C)C)CC3)cccc21] | 3.2 | 32 | B |
| 208 | 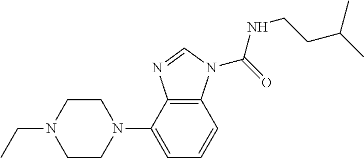 [SMILES: CCN1CCN(c2cccc3c2ncn3C(=O)NCCC(C)C)CC1] | 3.0 | 31 | B |
| 209 | 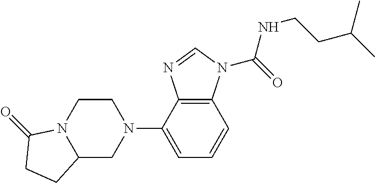 [SMILES: CC(C)CCNC(=O)n1cnc2c(N3CCN4C(=O)CCC4C3)cccc21] | 1.5 | 24 | B |
| 210 | 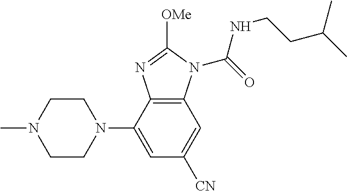 [SMILES: COc1nc2c(N3CCN(C)CC3)cc(C#N)cc2n1C(=O)NCCC(C)C] | 2.7 | 12 | A |
| 211 | 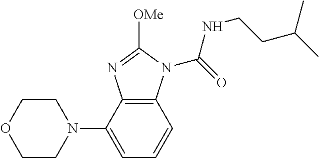 [SMILES: COc1nc2c(N3CCOCC3)cccc2n1C(=O)NCCC(C)C] | 1.9 | 26 | A |
| 212 | 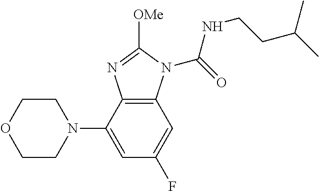 [SMILES: COc1nc2c(N3CCOCC3)cc(F)cc2n1C(=O)NCCC(C)C] | 2.8 | 8 | B |
| 213a | 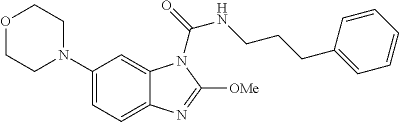 [SMILES: COc1nc2ccc(N3CCOCC3)cc2n1C(=O)NCCCc1ccccc1] | 2.4 | 9 | A |
| 213b | 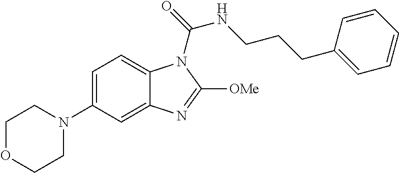 [SMILES: COc1nc2cc(N3CCOCC3)ccc2n1C(=O)NCCCc1ccccc1] | 2.4 | 16 | A |
| 214 | 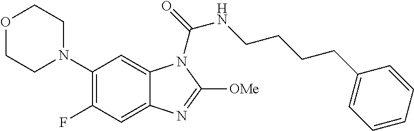 [SMILES: COc1nc2cc(F)c(N3CCOCC3)cc2n1C(=O)NCCCCc1ccccc1] | 3.8 | 0.3 | A |
| 215 | 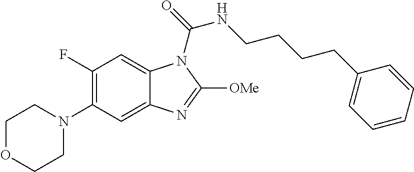 [SMILES: COc1nc2cc(N3CCOCC3)c(F)cc2n1C(=O)NCCCCc1ccccc1] | 3.8 | 0.3 | A |
| 216 | 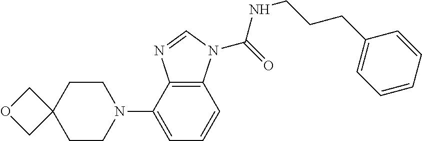 [SMILES: O=C(NCCCc1ccccc1)n1cnc2c(N3CCC4(CC3)COC4)cccc21] | 2.2 | 2.5 | A |
| 217 | 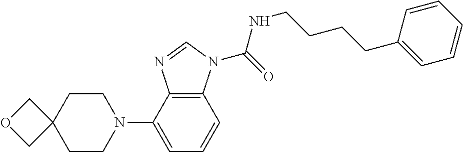 [SMILES: O=C(NCCCCc1ccccc1)n1cnc2c(N3CCC4(CC3)COC4)cccc21] | 2.8 | 0.6 | A |
| 218 | 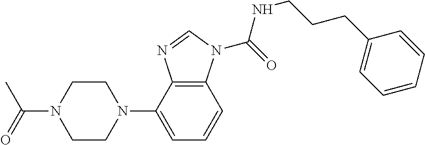 [SMILES: CC(=O)N1CCN(c2cccc3c2ncn3C(=O)NCCCc2ccccc2)CC1] | 2.0 | 24 | A |
| 219 | 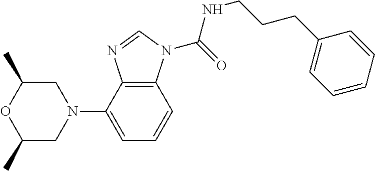 [SMILES: C[C@@H]1CN(c2cccc3c2ncn3C(=O)NCCCc2ccccc2)C[C@H](C)O1] | 3.4 | 0.5 | A |
| 220 | 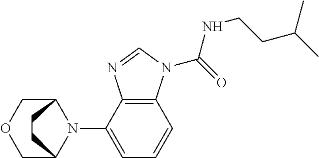 [SMILES: CC(C)CCNC(=O)n1cnc2c(N3[C@@H]4CC[C@H]3COC4)cccc21] | 2.2 | 4 | B |
| 221 | 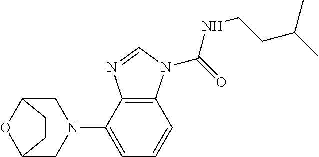 [SMILES: CC(C)CCNC(=O)n1cnc2c(N3CC4CCC(C3)O4)cccc21] | 2.3 | 5 | B |
| 221a | 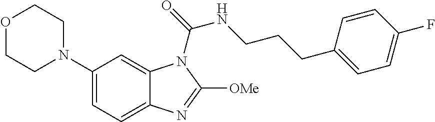 [SMILES: COc1nc2ccc(N3CCOCC3)cc2n1C(=O)NCCCc1ccc(F)cc1] | 3.3 | 4 | A |
| 221b | 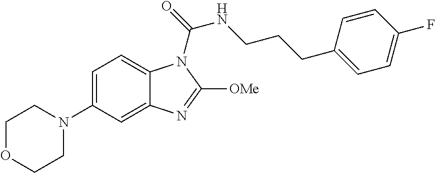 [SMILES: COc1nc2cc(N3CCOCC3)ccc2n1C(=O)NCCCc1ccc(F)cc1] | 3.3 | 15 | A |
| 222 | 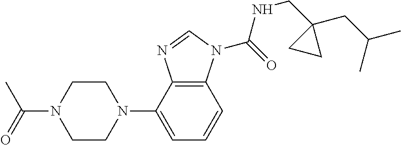 [SMILES: CC(=O)N1CCN(c2cccc3c2ncn3C(=O)NCC2(CC(C)C)CC2)CC1] | 2.4 | 7 | A |
| 223 | 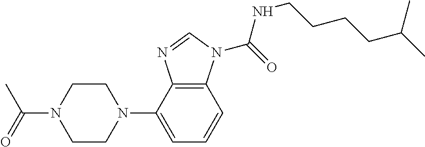 [SMILES: CC(=O)N1CCN(c2cccc3c2ncn3C(=O)NCCCCC(C)C)CC1] | 2.5 | 15 | A |
| 224a | 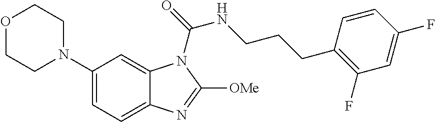 [SMILES: COc1nc2ccc(N3CCOCC3)cc2n1C(=O)NCCCc1ccc(F)cc1F] | 3.4 | 4 | A |
| 224b | 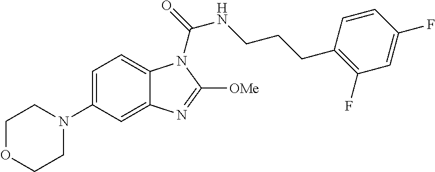 [SMILES: COc1nc2cc(N3CCOCC3)ccc2n1C(=O)NCCCc1ccc(F)cc1F] | 3.4 | 12 | A |
| 225 | 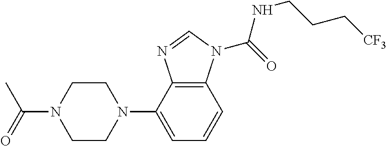 [SMILES: CC(=O)N1CCN(c2cccc3c2ncn3C(=O)NCCCC(F)(F)F)CC1] | 0.6 | 8 | A |
| 226 | 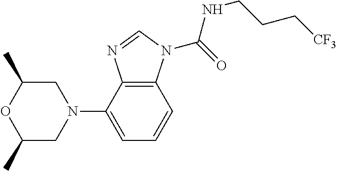 [SMILES: C[C@@H]1CN(c2cccc3c2ncn3C(=O)NCCCC(F)(F)F)C[C@H](C)O1] | 2.0 | 5 | B |
| 227 | 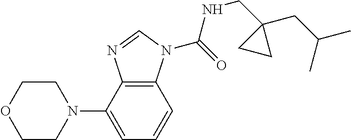 [SMILES: CC(C)CC1(CNC(=O)n2cnc3c(N4CCOCC4)cccc32)CC1] | 2.8 | 16 | A |
| 228a | 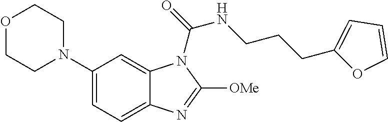 [SMILES: COc1nc2ccc(N3CCOCC3)cc2n1C(=O)NCCCc1ccco1] | 2.3 | 29 | B |
| 228b | 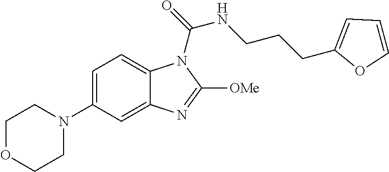 [SMILES: COc1nc2cc(N3CCOCC3)ccc2n1C(=O)NCCCc1ccco1] | 2.3 | 33 | B |
| 229a | 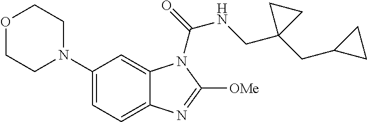 [SMILES: COc1nc2ccc(N3CCOCC3)cc2n1C(=O)NCC1(CC2CC2)CC1] | 3.1 | 1 | B |
| 229b | 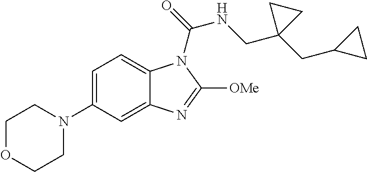 [SMILES: COc1nc2cc(N3CCOCC3)ccc2n1C(=O)NCC1(CC2CC2)CC1] | 3.1 | 9 | A |
| 230a | 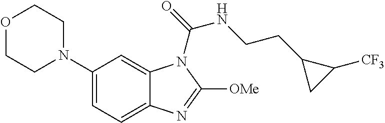 [SMILES: COc1nc2ccc(N3CCOCC3)cc2n1C(=O)NCCC1CC1C(F)(F)F] | 2.2 | 26 | B |
| 230b | 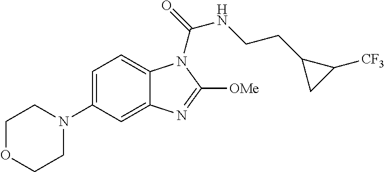 [SMILES: COc1nc2cc(N3CCOCC3)ccc2n1C(=O)NCCC1CC1C(F)(F)F] | 2.2 | 32 | B |
| 231a | 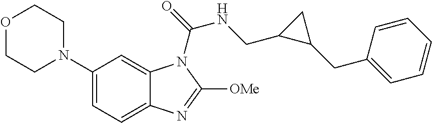 [SMILES: COc1nc2ccc(N3CCOCC3)cc2n1C(=O)NCC1CC1Cc1ccccc1] | 3.6 | 14 | B |
| 231b | 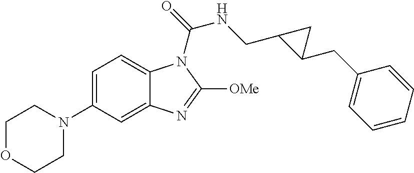 [SMILES: COc1nc2cc(N3CCOCC3)ccc2n1C(=O)NCC1CC1Cc1ccccc1] | 3.6 | 13 | A |
| 232a | 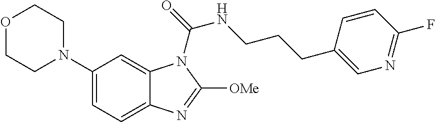 [SMILES: COc1nc2ccc(N3CCOCC3)cc2n1C(=O)NCCCc1ccc(F)nc1] | 1.9 | 25 | C |
| 232b | 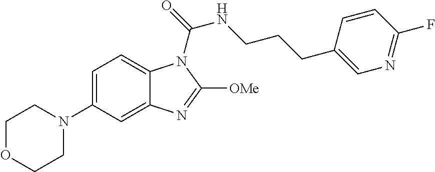 [SMILES: COc1nc2cc(N3CCOCC3)ccc2n1C(=O)NCCCc1ccc(F)nc1] | 1.9 | 25 | C |
| 233a | 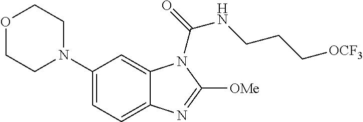 [SMILES: COc1nc2ccc(N3CCOCC3)cc2n1C(=O)NCCCOC(F)(F)F] | 2.3 | 30 | B |
| 233b | 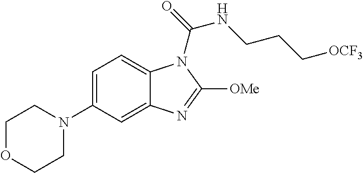 [SMILES: COc1nc2cc(N3CCOCC3)ccc2n1C(=O)NCCCOC(F)(F)F] | 2.3 | 29 | B |
| 234 | 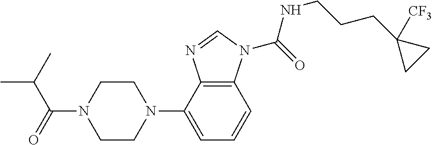 [SMILES: CC(C)C(=O)N1CCN(c2cccc3c2ncn3C(=O)NCCCC2(C(F)(F)F)CC2)CC1] | 2.1 | 1 | A |
| 235 | 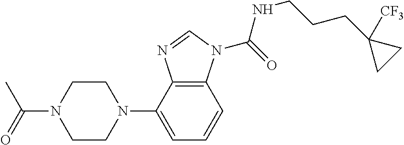 [SMILES: CC(=O)N1CCN(c2cccc3c2ncn3C(=O)NCCCC2(C(F)(F)F)CC2)CC1] | 1.3 | 20 | A |
| 236a | 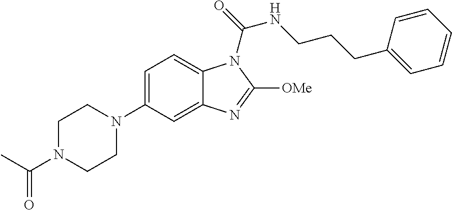 [SMILES: COc1nc2cc(N3CCN(C(C)=O)CC3)ccc2n1C(=O)NCCCc1ccccc1] | 2.7 | 18 | A |
| 236b | 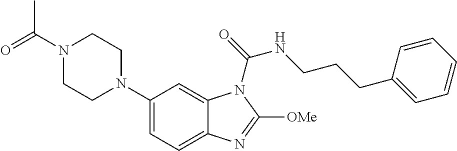 [SMILES: COc1nc2ccc(N3CCN(C(C)=O)CC3)cc2n1C(=O)NCCCc1ccccc1] | 2.7 | 23 | A |
| 237a | 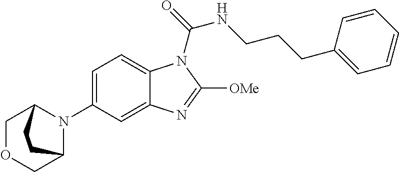 | 3.5 | 1 | A |
| 237b | 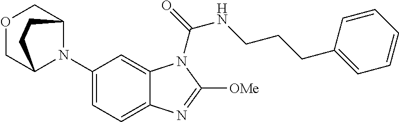 | 3.5 | 5 | A |
| 238a | 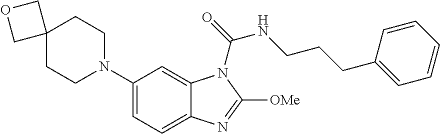 [SMILES: COc1nc2ccc(N3CCC4(CC3)COC4)cc2n1C(=O)NCCCc1ccccc1] | 3.1 | 11 | A |
| 238b | 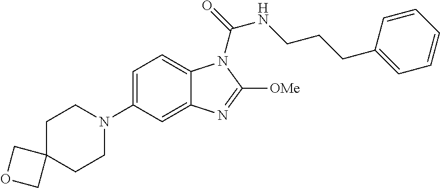 [SMILES: COc1nc2cc(N3CCC4(CC3)COC4)ccc2n1C(=O)NCCCc1ccccc1] | 3.0 | 12 | B |
| 239 | 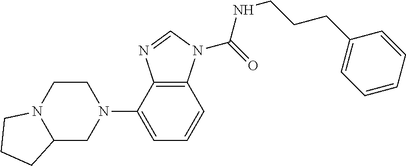 [SMILES: O=C(NCCCc1ccccc1)n1cnc2c(N3CCN4CCCC4C3)cccc21] | 3.2 | 17 | A |
| 240 | 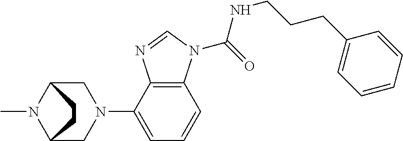 | 3.2 | 28 | B |
| 241 | 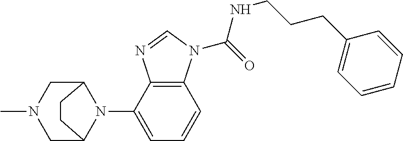 [SMILES: CN1CC2CCC(C1)N2c1cccc2c1ncn2C(=O)NCCCc1ccccc1] | 3.2 | 25 | A |
| 242 | 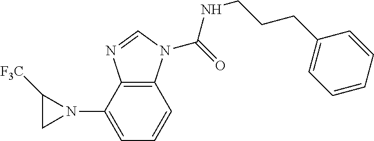 [SMILES: O=C(NCCCc1ccccc1)n1cnc2c(N3CC3C(F)(F)F)cccc21] | 3.1 | 1.0 | A |
| 243 | 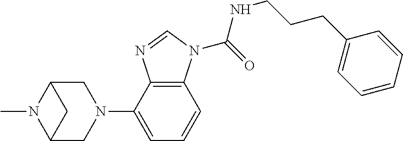 [SMILES: CN1C2CC1CN(c1cccc3c1ncn3C(=O)NCCCc1ccccc1)C2] | 2.7 | 28 | A |
| 244 | 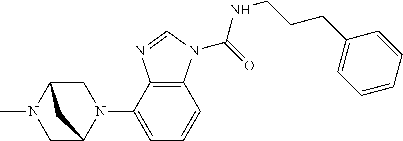 [SMILES: CN1C[C@@H]2C[C@H]1CN2c1cccc2c1ncn2C(=O)NCCCc1ccccc1] | 3.1 | 30 | A |
| 245 | 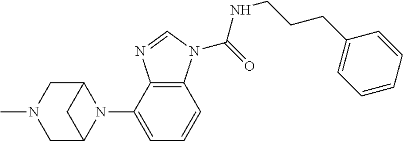 [SMILES: CN1CC2CC(C1)N2c1cccc2c1ncn2C(=O)NCCCc1ccccc1] | 2.7 | 32 | A |
| 246 | 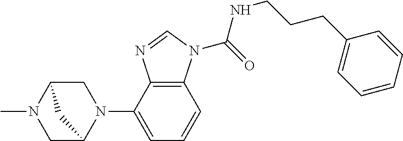 [SMILES: CN1C[C@H]2C[C@@H]1CN2c1cccc2c1ncn2C(=O)NCCCc1ccccc1] | 3.1 | 29 | A |
| 247 | 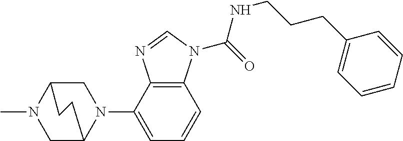 [SMILES: CN1CC2CCC1CN2c1cccc2c1ncn2C(=O)NCCCc1ccccc1] | 3.2 | 28 | A |
| 248 | 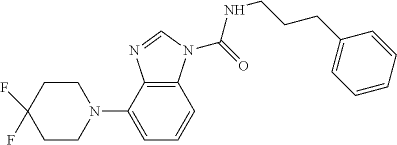 [SMILES: O=C(NCCCc1ccccc1)n1cnc2c(N3CCC(F)(F)CC3)cccc21] | 3.1 | 1 | A |
| 249 | 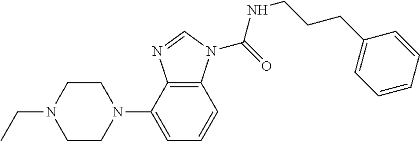 [SMILES: CCN1CCN(c2cccc3c2ncn3C(=O)NCCCc2ccccc2)CC1] | 3.5 | 12 | A |
| 250 | 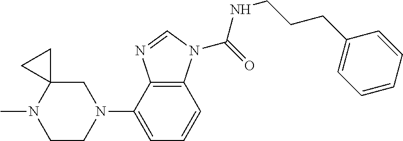 [SMILES: CN1CCN(c2cccc3c2ncn3C(=O)NCCCc2ccccc2)CC12CC2] | 3.2 | 23 | A |
| 251 | 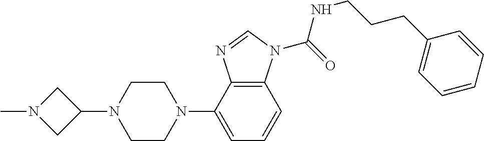 [SMILES: CN1CC(N2CCN(c3cccc4c3ncn4C(=O)NCCCc3ccccc3)CC2)C1] | 1.8 | 35 | A |
| 252 | 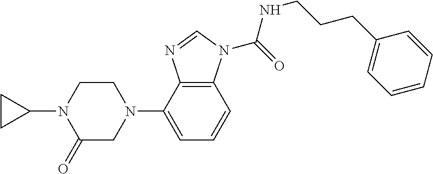 [SMILES: O=C1CN(c2cccc3c2ncn3C(=O)NCCCc2ccccc2)CCN1C1CC1] | 3.2 | 1.0 | A |
| 253a | 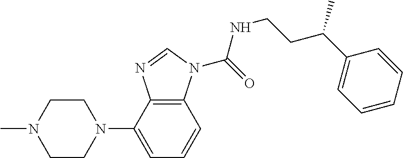 [SMILES: C[C@@H](CCNC(=O)n1cnc2c(N3CCN(C)CC3)cccc21)c1ccccc1] | 3.2 | 26 | A |
| 254 | 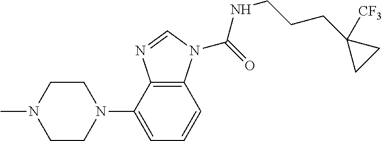 [SMILES: CN1CCN(c2cccc3c2ncn3C(=O)NCCCC2(C(F)(F)F)CC2)CC1] | 2.3 | 20 | A |
| 255 | 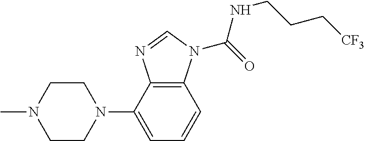 [SMILES: CN1CCN(c2cccc3c2ncn3C(=O)NCCCC(F)(F)F)CC1] | 1.6 | 35 | B |
| 256 | 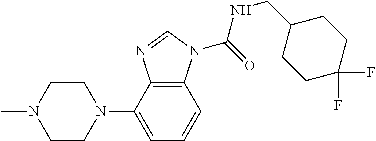 [SMILES: CN1CCN(c2cccc3c2ncn3C(=O)NCC2CCC(F)(F)CC2)CC1] | 2.5 | 24 | B |
| 257 | 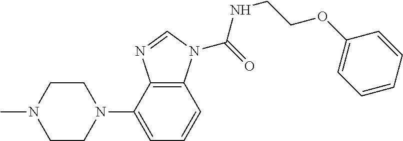 [SMILES: CN1CCN(c2cccc3c2ncn3C(=O)NCCOc2ccccc2)CC1] | 2.6 | 19 | A |
| 258 | 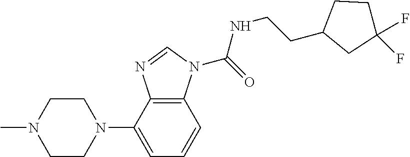 [SMILES: CN1CCN(c2cccc3c2ncn3C(=O)NCCC2CCC(F)(F)C2)CC1] | 2.5 | 34 | A |
| 259 | 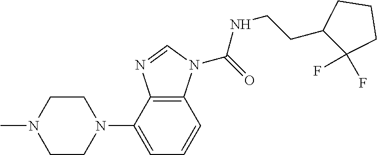 [SMILES: CN1CCN(c2cccc3c2ncn3C(=O)NCCC2CCCC2(F)F)CC1] | 2.5 | 21 | B |
| 260 | 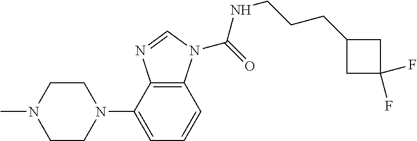 [SMILES: CN1CCN(c2cccc3c2ncn3C(=O)NCCCC2CC(F)(F)C2)CC1] | 2.4 | 31 | B |
| 261 | 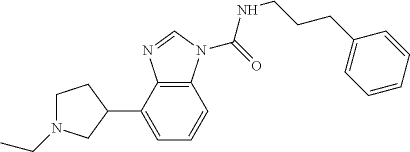 [SMILES: CCN1CCC(c2cccc3c2ncn3C(=O)NCCCc2ccccc2)C1] | 3.3 | 26 | A |
| 262 | 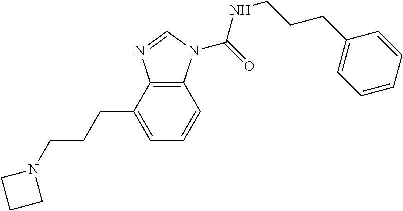 [SMILES: O=C(NCCCc1ccccc1)n1cnc2c(CCCN3CCC3)cccc21] | 3.1 | 21 | A |
| 263 | 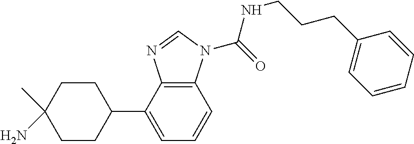 [SMILES: CC1(N)CCC(c2cccc3c2ncn3C(=O)NCCCc2ccccc2)CC1] | 2.3 | 25 | A |
| 264 | 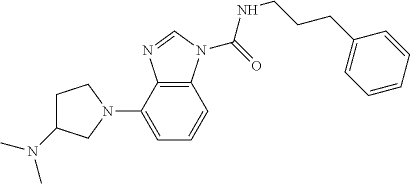 [SMILES: CN(C)C1CCN(c2cccc3c2ncn3C(=O)NCCCc2ccccc2)C1] | 2.8 | 21 | A |
| 265 | 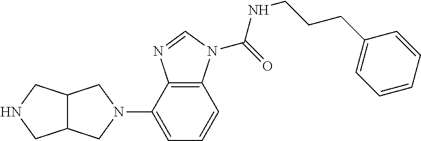 [SMILES: O=C(NCCCc1ccccc1)n1cnc2c(N3CC4CNCC4C3)cccc21] | 2.3 | 27 | A |
| 266 | 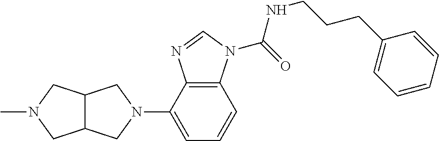 [SMILES: CN1CC2CN(c3cccc4c3ncn4C(=O)NCCCc3ccccc3)CC2C1] | 2.9 | 34 | A |
| 268 | 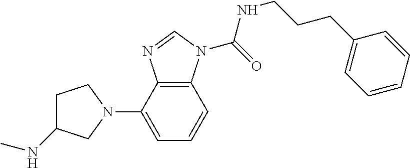 [SMILES: CNC1CCN(c2cccc3c2ncn3C(=O)NCCCc2ccccc2)C1] | 2.2 | 17 | A |
| 267 | 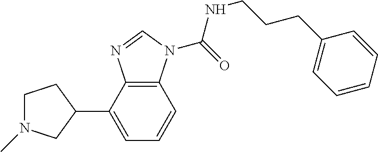 [SMILES: CN1CCC(c2cccc3c2ncn3C(=O)NCCCc2ccccc2)C1] | 2.8 | 25 | A |
| 269 | 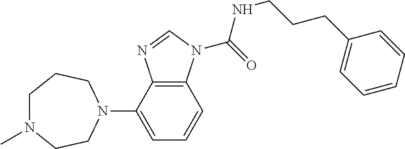 [SMILES: CN1CCCN(c2cccc3c2ncn3C(=O)NCCCc2ccccc2)CC1] | 2.9 | 21 | A |
| 270 | 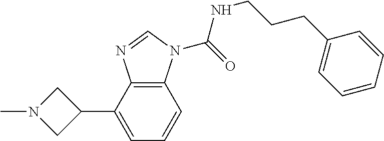 [SMILES: CN1CC(c2cccc3c2ncn3C(=O)NCCCc2ccccc2)C1] | 2.2 | 32 | A |
| 271 | 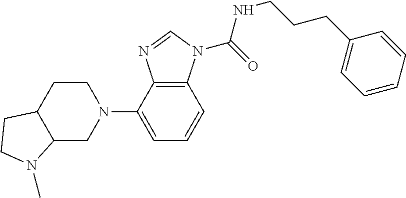 [SMILES: CN1CCC2CCN(c3cccc4c3ncn4C(=O)NCCCc3ccccc3)CC21] | 3.2 | 22 | A |
| 272 | 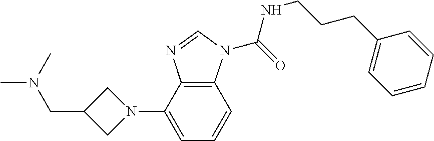 [SMILES: CN(C)CC1CN(c2cccc3c2ncn3C(=O)NCCCc2ccccc2)C1] | 2.0 | 21 | A |
| 273 | 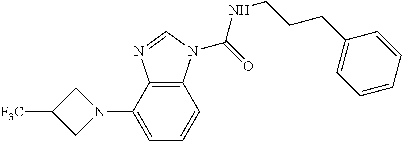 [SMILES: O=C(NCCCc1ccccc1)n1cnc2c(N3CC(C(F)(F)F)C3)cccc21] | 3.1 | 0.2 | A |
| 274 | 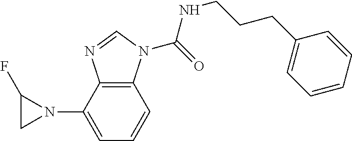 [SMILES: O=C(NCCCc1ccccc1)n1cnc2c(N3CC3F)cccc21] | 2.8 | 17 | A |
| 275 | 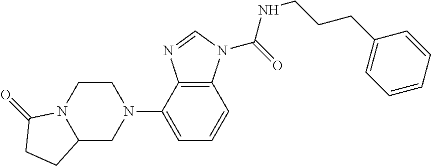 [SMILES: O=C1CCC2CN(c3cccc4c3ncn4C(=O)NCCCc3ccccc3)CCN12] | 2.0 | 15 | A |
| 276 | 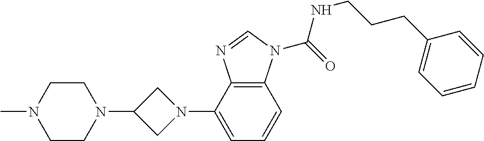 [SMILES: CN1CCN(C2CN(c3cccc4c3ncn4C(=O)NCCCc3ccccc3)C2)CC1] | 1.7 | 28 | A |
| 277 | 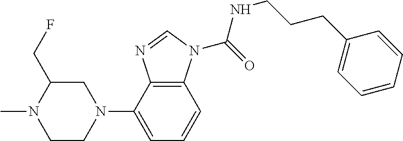 [SMILES: CN1CCN(c2cccc3c2ncn3C(=O)NCCCc2ccccc2)CC1CF] | 3.3 | 18 | A |
| 278 | 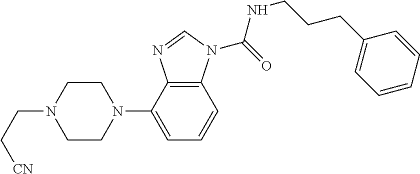 [SMILES: N#CCCN1CCN(c2cccc3c2ncn3C(=O)NCCCc2ccccc2)CC1] | 2.6 | 18 | A |
| 279 | 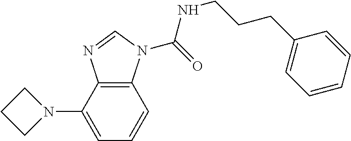 [SMILES: O=C(NCCCc1ccccc1)n1cnc2c(N3CCC3)cccc21] | 2.6 | 1 | A |
| 280 | 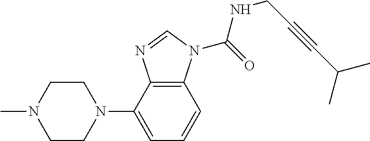 [SMILES: CC(C)C#CCNC(=O)n1cnc2c(N3CCN(C)CC3)cccc21] | 1.9 | 16 | A |
| 281 | 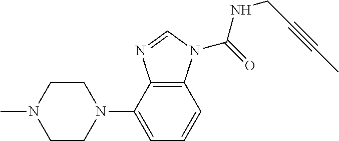 [SMILES: CC#CCNC(=O)n1cnc2c(N3CCN(C)CC3)cccc21] | 1.0 | 32 | B |
| 282 | 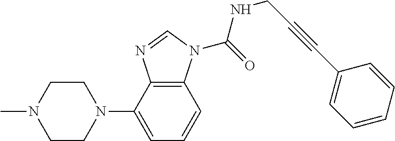 [SMILES: CN1CCN(c2cccc3c2ncn3C(=O)NCC#Cc2ccccc2)CC1] | 2.2 | 21 | A |
| 283 | 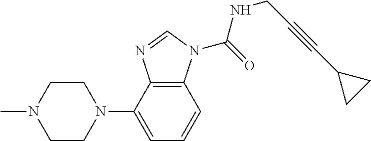 [SMILES: CN1CCN(c2cccc3c2ncn3C(=O)NCC#CC2CC2)CC1] | 1.5 | 28 | A |
| 284 | 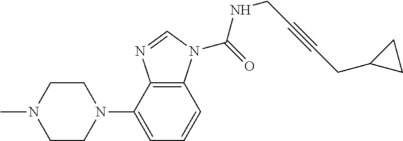 [SMILES: CN1CCN(c2cccc3c2ncn3C(=O)NCC#CCC2CC2)CC1] | 2.1 | 30 | A |
| 285 | 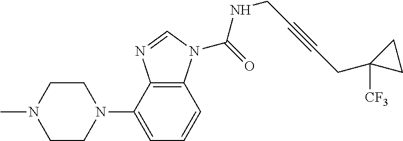 [SMILES: CN1CCN(c2cccc3c2ncn3C(=O)NCC#CCC2(C(F)(F)F)CC2)CC1] | 1.9 | 22 | A |
| 253b | 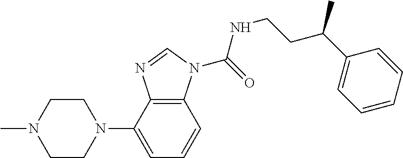 [SMILES: C[C@H](CCNC(=O)n1cnc2c(N3CCN(C)CC3)cccc21)c1ccccc1] | 3.2 | 27 | A |
| 286 | 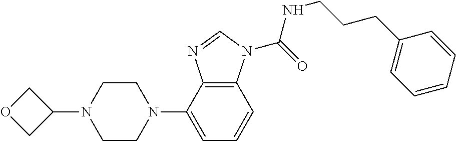 [SMILES: O=C(NCCCc1ccccc1)n1cnc2c(N3CCN(C4COC4)CC3)cccc21] | 2.5 | 24 | A |
| 287 | 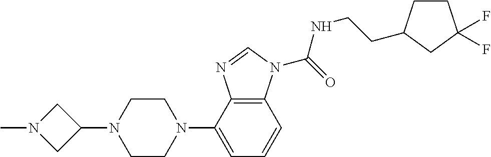 [SMILES: CN1CC(N2CCN(c3cccc4c3ncn4C(=O)NCCC3CCC(F)(F)C3)CC2)C1] | 1.3 | 36 | B |
| 288 | 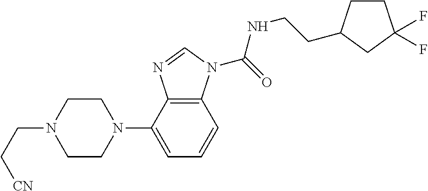 [SMILES: N#CCCN1CCN(c2cccc3c2ncn3C(=O)NCCC2CCC(F)(F)C2)CC1] | 2.0 | 26 | B |
| 289 | 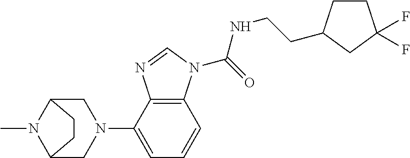 [SMILES: CN1C2CCC1CN(c1cccc3c1ncn3C(=O)NCCC1CCC(F)(F)C1)C2] | 2.8 | 39 | B |
| 290 | 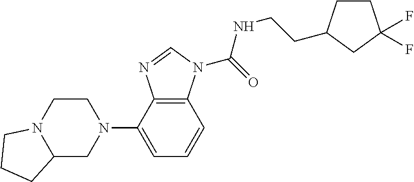 [SMILES: O=C(NCCC1CCC(F)(F)C1)n1cnc2c(N3CCN4CCCC4C3)cccc21] | 2.7 | 28 | B |
| 291 | 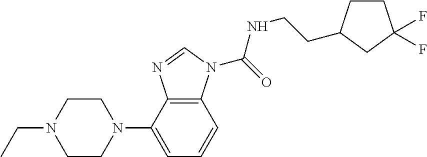 [SMILES: CCN1CCN(c2cccc3c2ncn3C(=O)NCCC2CCC(F)(F)C2)CC1] | 3.0 | 20 | B |
| 292 | 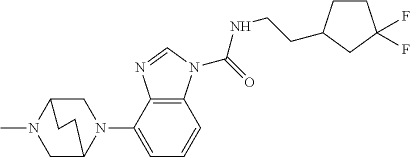 [SMILES: CN1CC2CCC1CN2c1cccc2c1ncn2C(=O)NCCC1CCC(F)(F)C1] | 2.8 | 36 | B |
| 293 | 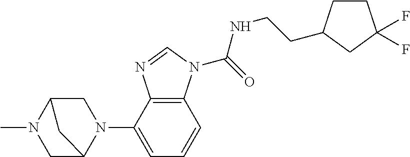 [SMILES: CN1CC2CC1CN2c1cccc2c1ncn2C(=O)NCCC1CCC(F)(F)C1] | 2.6 | 39 | B |
| 294 | 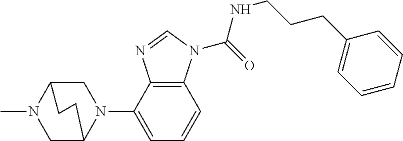 [SMILES: CN1CC2CCC1CN2c1cccc2c1ncn2C(=O)NCCCc1ccccc1] | 2.9 | 32 | A |
| 295 | 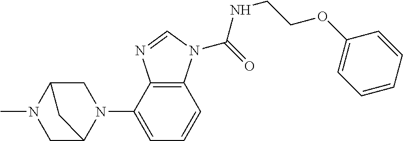 [SMILES: CN1CC2CC1CN2c1cccc2c1ncn2C(=O)NCCOc1ccccc1] | 2.8 | 33 | A |
| 296 | 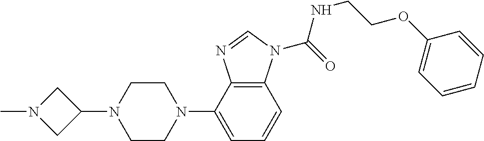 [SMILES: CN1CC(N2CCN(c3cccc4c3ncn4C(=O)NCCOc3ccccc3)CC2)C1] | 1.5 | 36 | A |
| 297 | 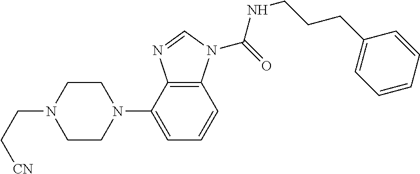 [SMILES: N#CCCN1CCN(c2cccc3c2ncn3C(=O)NCCCc2ccccc2)CC1] | 2.3 | 23 | A |
| 298 | 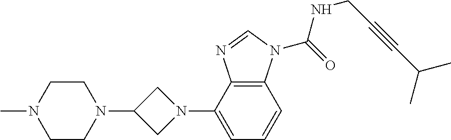 [SMILES: CC(C)C#CCNC(=O)n1cnc2c(N3CC(N4CCN(C)CC4)C3)cccc21] | 0.6 | 27 | A |
| 299 | 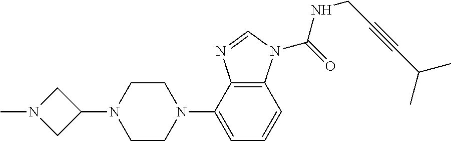 [SMILES: CC(C)C#CCNC(=O)n1cnc2c(N3CCN(C4CN(C)C4)CC3)cccc21] | 0.6 | 30 | A |
| 300 | 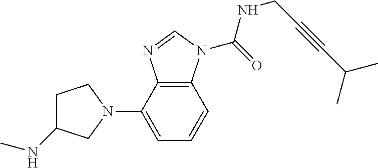 [SMILES: CNC1CCN(c2cccc3c2ncn3C(=O)NCC#CC(C)C)C1] | 1.3 | 29 | A |
| 301 | 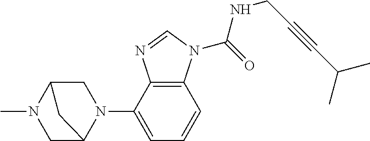 [SMILES: CC(C)C#CCNC(=O)n1cnc2c(N3CC4CC3CN4C)cccc21] | 2.0 | 34 | B |
| 302 | 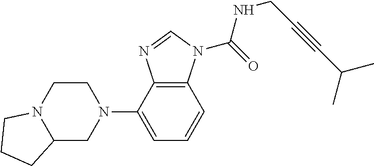 [SMILES: CC(C)C#CCNC(=O)n1cnc2c(N3CCN4CCCC4C3)cccc21] | 2.2 | 22 | A |
| 303 | 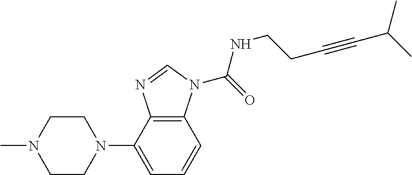 [SMILES: CC(C)C#CCCNC(=O)n1cnc2c(N3CCN(C)CC3)cccc21] | 2.4 | 28 | B |
| 304 | 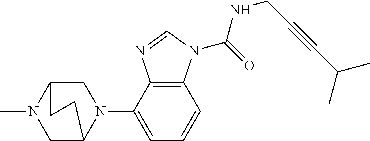 [SMILES: CC(C)C#CCNC(=O)n1cnc2c(N3CC4CCC3CN4C)cccc21] | 2.2 | 27 | B |
| 305 | 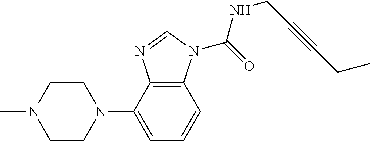 [SMILES: CCC#CCNC(=O)n1cnc2c(N3CCN(C)CC3)cccc21] | 1.5 | 27 | B |
| 306 | 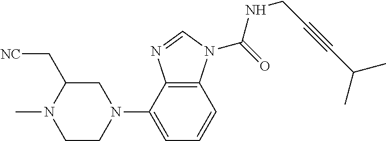 [SMILES: CC(C)C#CCNC(=O)n1cnc2c(N3CCN(C)C(CC#N)C3)cccc21] | 2.2 | 23 | A |
| 307 | 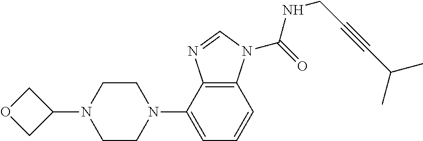 [SMILES: CC(C)C#CCNC(=O)n1cnc2c(N3CCN(C4COC4)CC3)cccc21] | 1.6 | 20 | A |
| 308 | 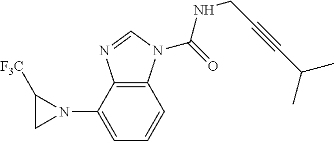 [SMILES: CC(C)C#CCNC(=O)n1cnc2c(N3CC3C(F)(F)F)cccc21] | 2.2 | 1 | A |
| 309 | 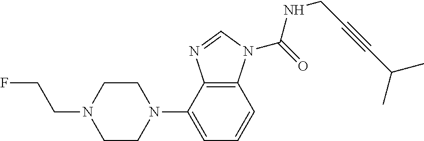 [SMILES: CC(C)C#CCNC(=O)n1cnc2c(N3CCN(CCF)CC3)cccc21] | 2.1 | 22 | A |
| 310 | 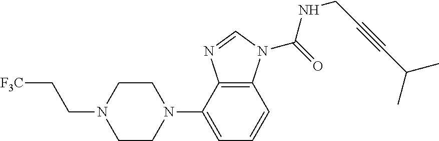 [SMILES: CC(C)C#CCNC(=O)n1cnc2c(N3CCN(CCC(F)(F)F)CC3)cccc21] | 2.7 | 4 | A |
| 311 | 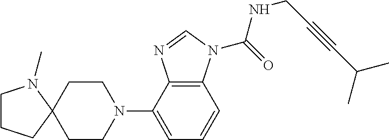 [SMILES: CC(C)C#CCNC(=O)n1cnc2c(N3CCC4(CCCN4C)CC3)cccc21] | 2.1 | 31 | A |
| 312 | 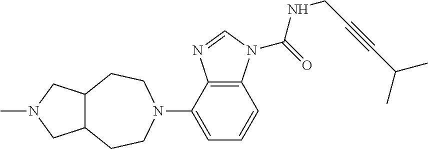 [SMILES: CC(C)C#CCNC(=O)n1cnc2c(N3CCC4CN(C)CC4CC3)cccc21] | 2.1 | 24 | A |
| 313 | 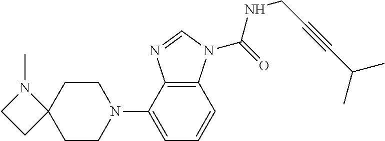 [SMILES: CC(C)C#CCNC(=O)n1cnc2c(N3CCC4(CC3)CCN4C)cccc21] | 1.6 | 29 | A |
| 314 | 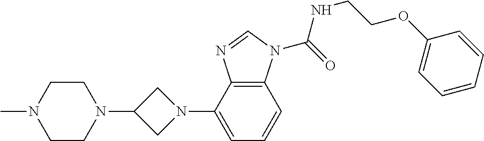 [SMILES: CN1CCN(C2CN(c3cccc4c3ncn4C(=O)NCCOc3ccccc3)C2)CC1] | 1.3 | 35 | A |
| 315 | 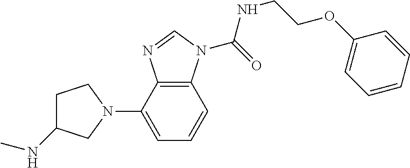 [SMILES: CNC1CCN(c2cccc3c2ncn3C(=O)NCCOc2ccccc2)C1] | 1.4 | 30 | A |
| 316 | 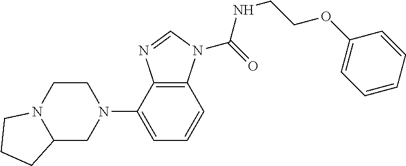 [SMILES: O=C(NCCOc1ccccc1)n1cnc2c(N3CCN4CCCC4C3)cccc21] | 2.9 | 27 | A |
| 317 | 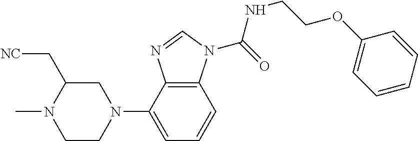 [SMILES: CN1CCN(c2cccc3c2ncn3C(=O)NCCOc2ccccc2)CC1CC#N] | 2.3 | 26 | A |
| 318 | 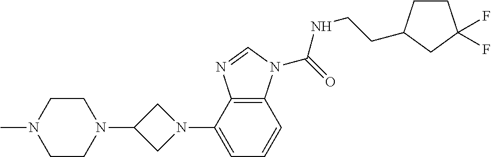 [SMILES: CN1CCN(C2CN(c3cccc4c3ncn4C(=O)NCCC3CCC(F)(F)C3)C2)CC1] | 1.2 | 40 | B |
| 319a | 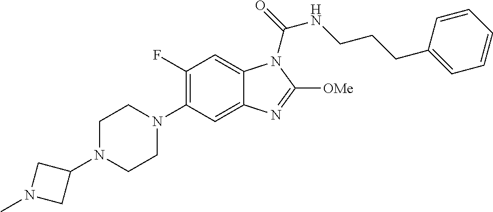 [SMILES: COc1nc2cc(N3CCN(C4CN(C)C4)CC3)c(F)cc2n1C(=O)NCCCc1ccccc1] | 2.4 | 35 | B |
| 319b | 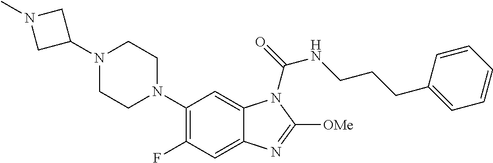 [SMILES: COc1nc2cc(F)c(N3CCN(C4CN(C)C4)CC3)cc2n1C(=O)NCCCc1ccccc1] | 2.4 | 46 | B |
| 320a | 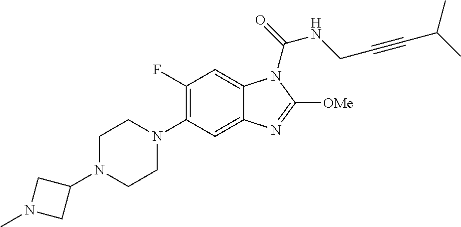 [SMILES: COc1nc2cc(N3CCN(C4CN(C)C4)CC3)c(F)cc2n1C(=O)NCC#CC(C)C] | 1.5 | 33 | C |
| 320b | 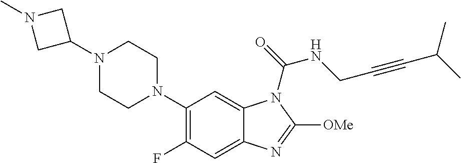 [SMILES: COc1nc2cc(F)c(N3CCN(C4CN(C)C4)CC3)cc2n1C(=O)NCC#CC(C)C] | 1.5 | 42 | B |
| 321a | 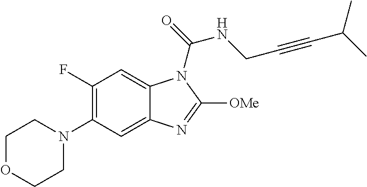 [SMILES: COc1nc2cc(N3CCOCC3)c(F)cc2n1C(=O)NCC#CC(C)C] | 2.4 | 2 | B |
| 321b | 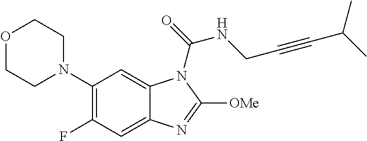 [SMILES: COc1nc2cc(F)c(N3CCOCC3)cc2n1C(=O)NCC#CC(C)C] | 2.4 | 3 | B |
| 322 | 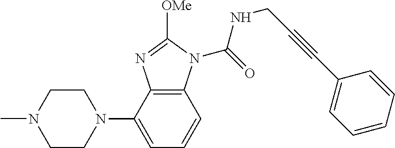 [SMILES: COc1nc2c(N3CCN(C)CC3)cccc2n1C(=O)NCC#Cc1ccccc1] | 2.8 | 22 | B |
| 323 | 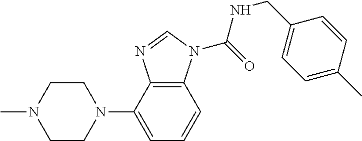 | 2.7 | 16 | A |
| 324 | 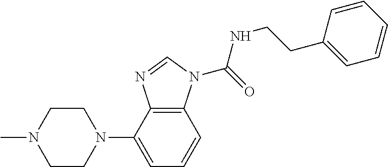 [SMILES: CN1CCN(c2cccc3c2ncn3C(=O)NCCc2ccccc2)CC1] | 2.6 | 11 | B |
| 325 | 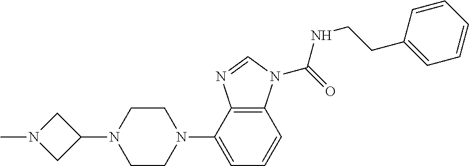 [SMILES: CN1CC(N2CCN(c3cccc4c3ncn4C(=O)NCCc3ccccc3)CC2)C1] | 1.4 | 32 | A |
| 326 | 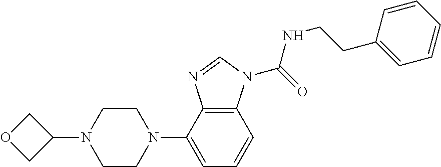 [SMILES: O=C(NCCc1ccccc1)n1cnc2c(N3CCN(C4COC4)CC3)cccc21] | 2.1 | 21 | A |
| 327 | 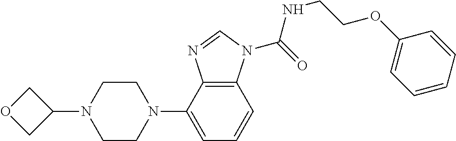 [SMILES: O=C(NCCOc1ccccc1)n1cnc2c(N3CCN(C4COC4)CC3)cccc21] | 2.2 | 25 | A |
| 328 | 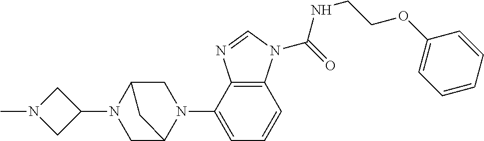 [SMILES: CN1CC(N2CC3CC2CN3c2cccc3c2ncn3C(=O)NCCOc2ccccc2)C1] | 1.6 | 41 | A |
| 329 | 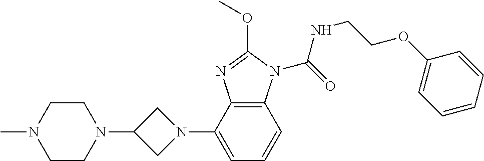 [SMILES: COc1nc2c(N3CC(N4CCN(C)CC4)C3)cccc2n1C(=O)NCCOc1ccccc1] | 2.1 | 38 | C |
| 330 | 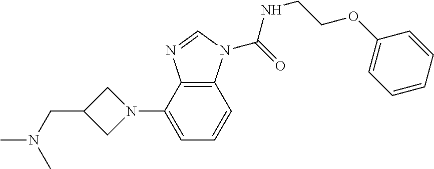 [SMILES: CN(C)CC1CN(c2cccc3c2ncn3C(=O)NCCOc2ccccc2)C1] | 1.6 | 38 | A |
| 331 | 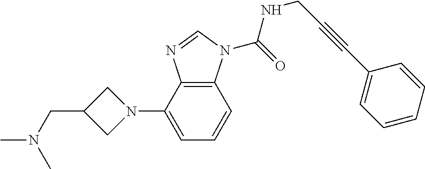 [SMILES: CN(C)CC1CN(c2cccc3c2ncn3C(=O)NCC#Cc2ccccc2)C1] | 1.2 | 22 | A |
| 332 | 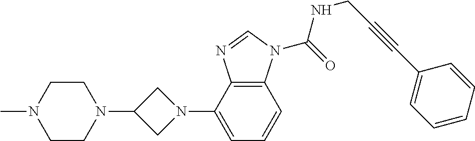 [SMILES: CN1CCN(C2CN(c3cccc4c3ncn4C(=O)NCC#Cc3ccccc3)C2)CC1] | 0.9 | 24 | A |
| 333 | 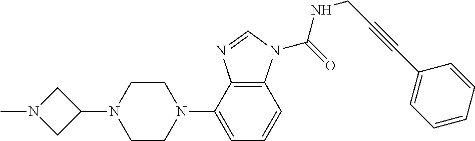 [SMILES: CN1CC(N2CCN(c3cccc4c3ncn4C(=O)NCC#Cc3ccccc3)CC2)C1] | 1.0 | 26 | A |
| 334a | 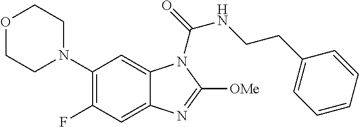 [SMILES: COc1nc2cc(F)c(N3CCOCC3)cc2n1C(=O)NCCc1ccccc1] | 2.9 | 3 | A |
| 334b | 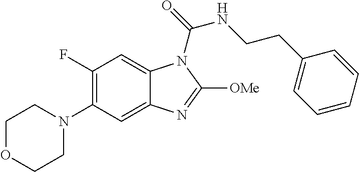 [SMILES: COc1nc2cc(N3CCOCC3)c(F)cc2n1C(=O)NCCc1ccccc1] | 2.9 | 5 | A |
| 335a | 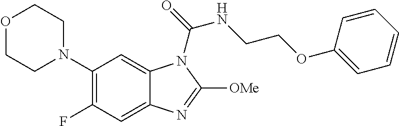 [SMILES: COc1nc2cc(F)c(N3CCOCC3)cc2n1C(=O)NCCOc1ccccc1] | 3.0 | 1 | A |
| 335b | 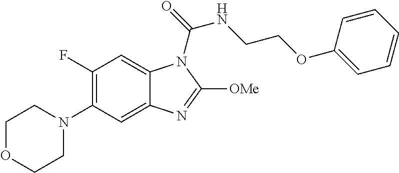 [SMILES: COc1nc2cc(N3CCOCC3)c(F)cc2n1C(=O)NCCOc1ccccc1] | 3.0 | 7 | A |
| 336 | 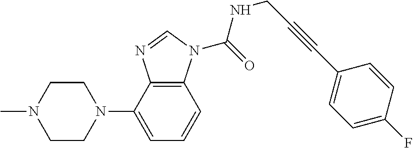 [SMILES: CN1CCN(c2cccc3c2ncn3C(=O)NCC#Cc2ccc(F)cc2)CC1] | 2.3 | 15 | A |
| 337 | 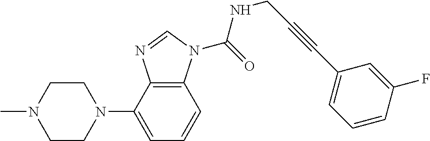 [SMILES: CN1CCN(c2cccc3c2ncn3C(=O)NCC#Cc2cccc(F)c2)CC1] | 2.3 | 15 | A |
| 338 | 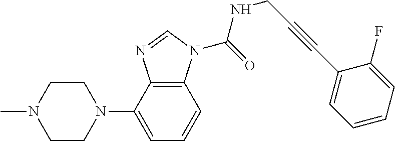 [SMILES: CN1CCN(c2cccc3c2ncn3C(=O)NCC#Cc2ccccc2F)CC1] | 2.3 | 18 | A |
| 339 | 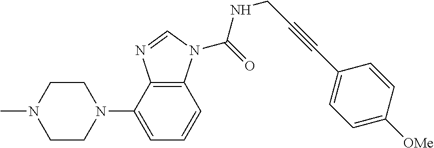 [SMILES: COc1ccc(C#CCNC(=O)n2cnc3c(N4CCN(C)CC4)cccc32)cc1] | 2.0 | 16 | A |
| 340 | 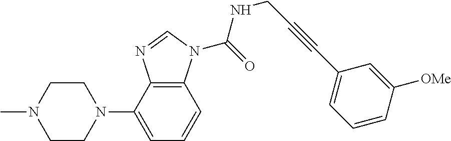 [SMILES: COc1cccc(C#CCNC(=O)n2cnc3c(N4CCN(C)CC4)cccc32)c1] | 2.0 | 13 | A |
| 341 | 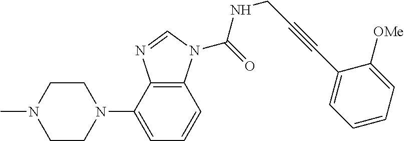 [SMILES: COc1ccccc1C#CCNC(=O)n1cnc2c(N3CCN(C)CC3)cccc21] | 2.0 | 20 | A |
| 342 | 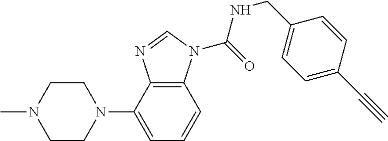 [SMILES: C#Cc1ccc(CNC(=O)n2cnc3c(N4CCN(C)CC4)cccc32)cc1] | 2.5 | 4 | A |
| 343 | 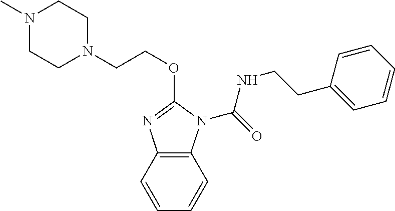 [SMILES: CN1CCN(CCOc2nc3ccccc3n2C(=O)NCCc2ccccc2)CC1] | 2.1 | 24 | B |
| 344 | 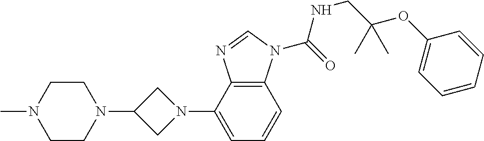 [SMILES: CN1CCN(C2CN(c3cccc4c3ncn4C(=O)NCC(C)(C)Oc3ccccc3)C2)CC1] | 2.1 | 29 | A |
| 345 | 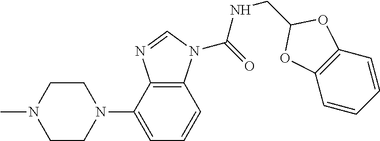 [SMILES: CN1CCN(c2cccc3c2ncn3C(=O)NCC2Oc3ccccc3O2)CC1] | 2.2 | 40 | A |
| 346 | 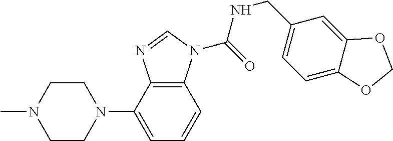 [SMILES: CN1CCN(c2cccc3c2ncn3C(=O)NCc2ccc3c(c2)OCO3)CC1] | 2.2 | 11 | A |
| 347 | 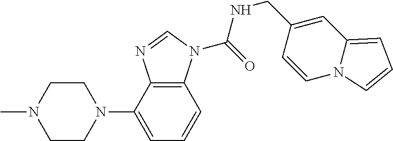 [SMILES: CN1CCN(c2cccc3c2ncn3C(=O)NCc2ccn3cccc3c2)CC1] | 2.7 | 25 | A |
| 348 | 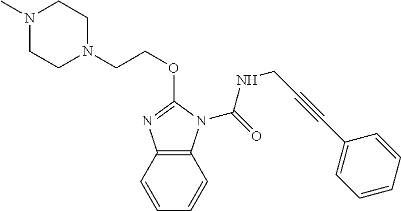 [SMILES: CN1CCN(CCOc2nc3ccccc3n2C(=O)NCC#Cc2ccccc2)CC1] | 1.8 | 8 | A |
| 349 | 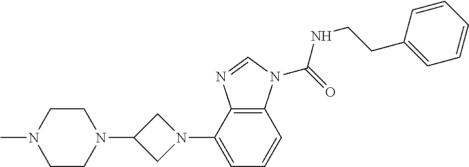 [SMILES: CN1CCN(C2CN(c3cccc4c3ncn4C(=O)NCCc3ccccc3)C2)CC1] | 1.3 | 37 | B |
| 350 | 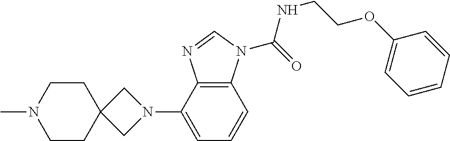 [SMILES: CN1CCC2(CC1)CN(c1cccc3c1ncn3C(=O)NCCOc1ccccc1)C2] | 2.4 | 35 | A |
| 351 | 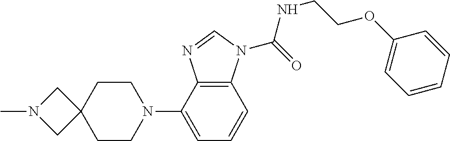 [SMILES: CN1CC2(CCN(c3cccc4c3ncn4C(=O)NCCOc3ccccc3)CC2)C1] | 2.4 | 33 | A |
| 352 | 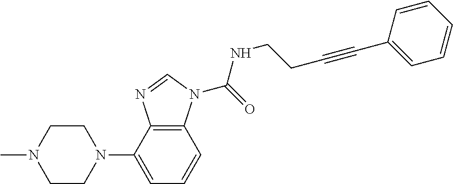 [SMILES: CN1CCN(c2cccc3c2ncn3C(=O)NCCC#Cc2ccccc2)CC1] | 2.7 | 2 | A |
| 353 | 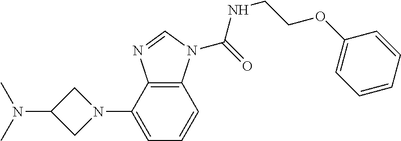 [SMILES: CN(C)C1CN(c2cccc3c2ncn3C(=O)NCCOc2ccccc2)C1] | 2.9 | 16 | A |
| 354 | 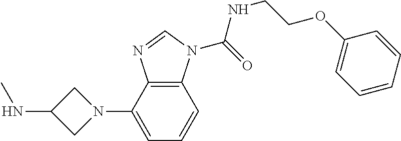 [SMILES: CNC1CN(c2cccc3c2ncn3C(=O)NCCOc2ccccc2)C1] | 2.2 | 19 | C |
| 355 | 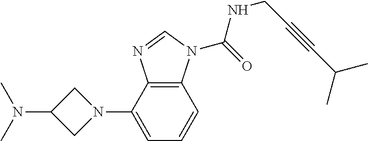 [SMILES: CC(C)C#CCNC(=O)n1cnc2c(N3CC(N(C)C)C3)cccc21] | 2.3 | 22 | B |
| 356 | 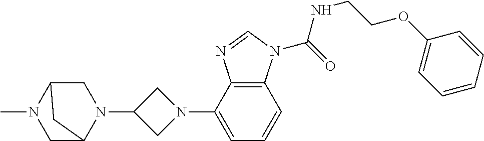 [SMILES: CN1CC2CC1CN2C1CN(c2cccc3c2ncn3C(=O)NCCOc2ccccc2)C1] | 1.1 | 46 | A |
| 357 | 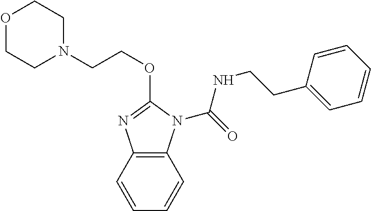 [SMILES: O=C(NCCc1ccccc1)n1c(OCCN2CCOCC2)nc2ccccc21] | 3.0 | 6 | B |
| 358 | 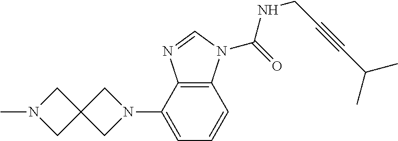 [SMILES: CC(C)C#CCNC(=O)n1cnc2c(N3CC4(CN(C)C4)C3)cccc21] | 2.0 | 33 | B |
| 359 | 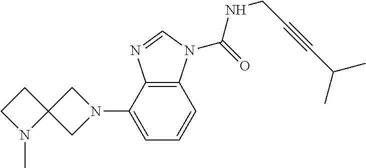 [SMILES: CC(C)C#CCNC(=O)n1cnc2c(N3CC4(CCN4C)C3)cccc21] | 1.6 | 23 | B |
| 360 | 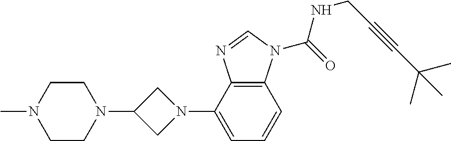 [SMILES: CN1CCN(C2CN(c3cccc4c3ncn4C(=O)NCC#CC(C)(C)C)C2)CC1] | 1.1 | 27 | A |
| 361 | 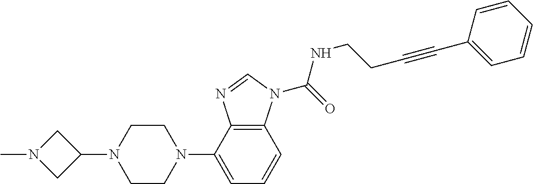 [SMILES: CN1CC(N2CCN(c3cccc4c3ncn4C(=O)NCCC#Cc3ccccc3)CC2)C1] | 1.6 | 24 | A |
| 362 | 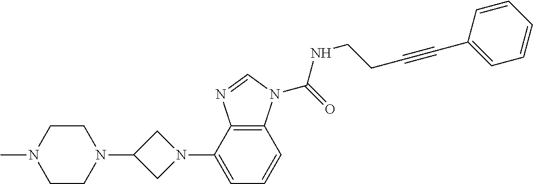 [SMILES: CN1CCN(C2CN(c3cccc4c3ncn4C(=O)NCCC#Cc3ccccc3)C2)CC1] | 1.4 | 23 | A |
| 363 | 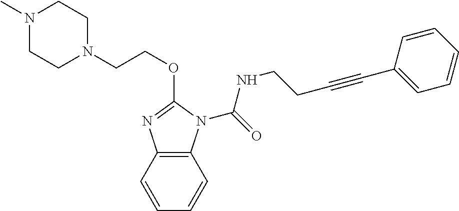 [SMILES: CN1CCN(CCOc2nc3ccccc3n2C(=O)NCCC#Cc2ccccc2)CC1] | 2.3 | 28 | A |
| 364 | 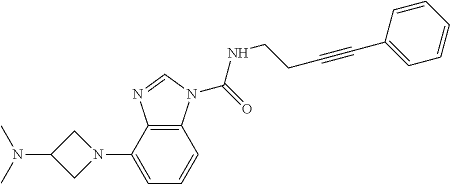 [SMILES: CN(C)C1CN(c2cccc3c2ncn3C(=O)NCCC#Cc2ccccc2)C1] | 2.9 | 17 | A |
| 365 | 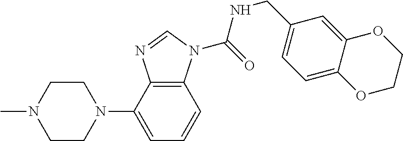 [SMILES: CN1CCN(c2cccc3c2ncn3C(=O)NCc2ccc3c(c2)OCCO3)CC1] | 2.1 | 11 | A |
| 366 | 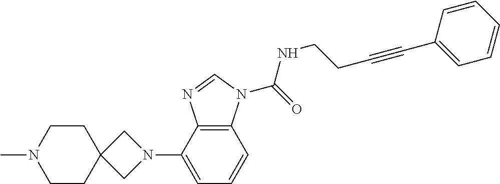 [SMILES: CN1CCC2(CC1)CN(c1cccc3c1ncn3C(=O)NCCC#Cc1ccccc1)C2] | 2.4 | 21 | A |
| 367 | 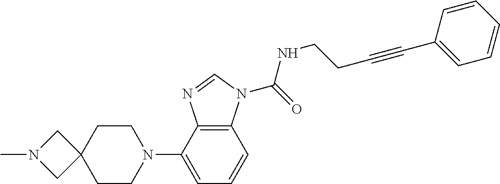 [SMILES: CN1CC2(CCN(c3cccc4c3ncn4C(=O)NCCC#Cc3ccccc3)CC2)C1] | 2.4 | 27 | A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound of formula (I): 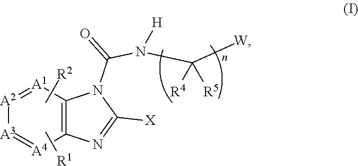
or a pharmaceutically acceptable salt thereof, wherein:
R¹ is selected from the group consisting of hydrogen, phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl);
R² is selected from the group consisting of hydrogen, halogen, C₁₋₆alkyl, C₁₋₆haloalkyl, —OR^{c}, and cyano;
R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene;
n is integer selected from 0 to 6;
X is selected from the group consisting of hydrogen, deuterium, —OR^{b}, —S(C₁₋₆alkyl), C₁₋₆ alkyl, and phenyl;
R^{a} is independently, for each occurrence, hydrogen or C₁₋₆alkyl;
R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂;
R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;
W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl; and
A¹, A², A³, and A⁴ are CH, or one or two of A¹, A², A³, and A⁴ are N and the others are CH; and
wherein any aforementioned phenyl, C₃₋₇cycloalkyl, 3-10 membered heterocyclyl, —(C₂₋₆alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted;
wherein, the compound is not a compound of 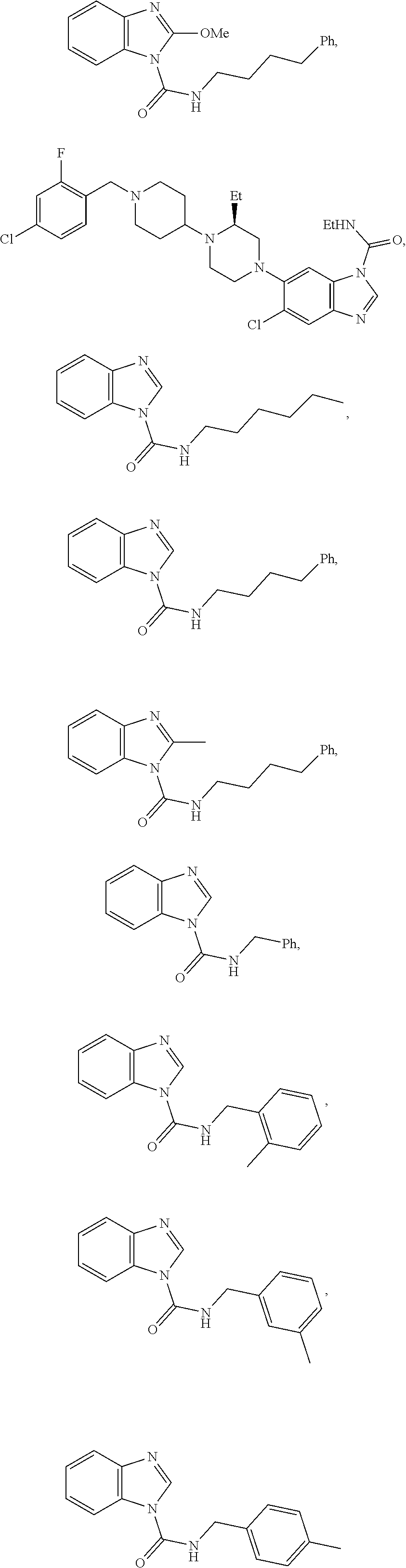
or a pharmaceutically acceptable salt thereof.

2. The compound of claim 1, wherein R¹ is selected from the group consisting of hydrogen, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl).

3. The compound of claim 1 or 2, wherein R¹ is selected from the group consisting of hydrogen, 3-10 membered heterocyclyl, 5-6 membered heteroaryl, C₁₋₆alkylene-(3-7 membered heterocyclyl), (3-7 membered heterocyclene)-(5-10 membered heteroaryl), (3-7 membered heterocyclene)-(C₃₋₇cycloalkyl), and (3-7 membered heterocyclene)-(3-7 membered heterocyclyl), wherein the 3-10 membered heterocyclyl, (3-7 membered heterocyclene)-(C₃₋₇cycloalkyl), and (3-7 membered heterocyclene)-(3-7 membered heterocyclyl) are optionally substituted with 1-3 substituents independently, for each occurrence, selected from the group consisting of halogen, cyano, oxo, C₁₋₆alkyl, —C₁₋₆alkylene-CN, C₁₋₆haloalkyl, —O—C₁₋₆alkyl, —N(R^{a})₂, —C(O)—C₁₋₄alkyl, and —C₁₋₄alkylene-(C₃₋₇cycloalkyl).

4. The compound of any one of claims 1-3, wherein R¹ is selected from the group consisting of hydrogen,

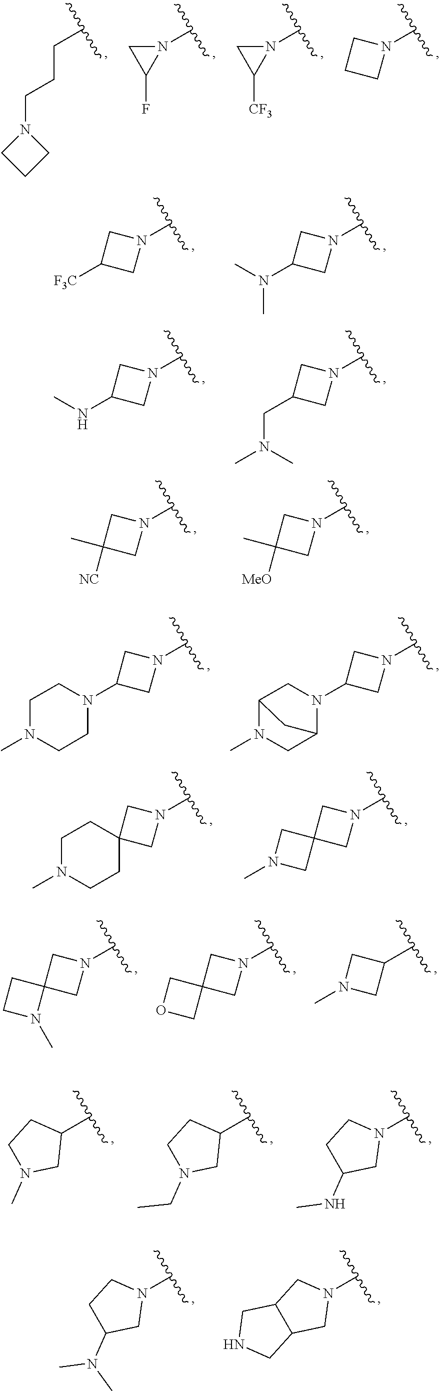

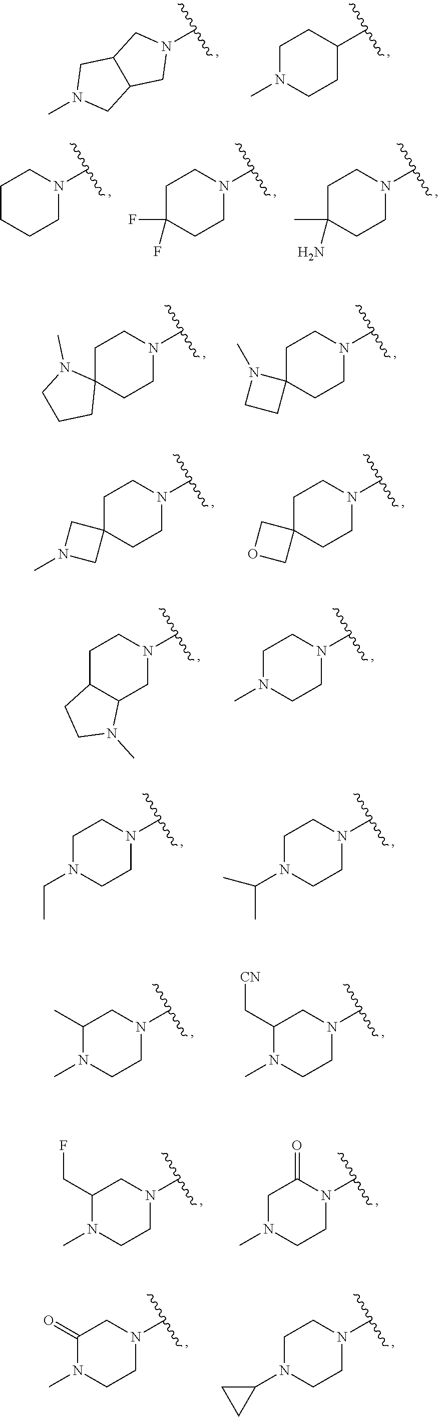

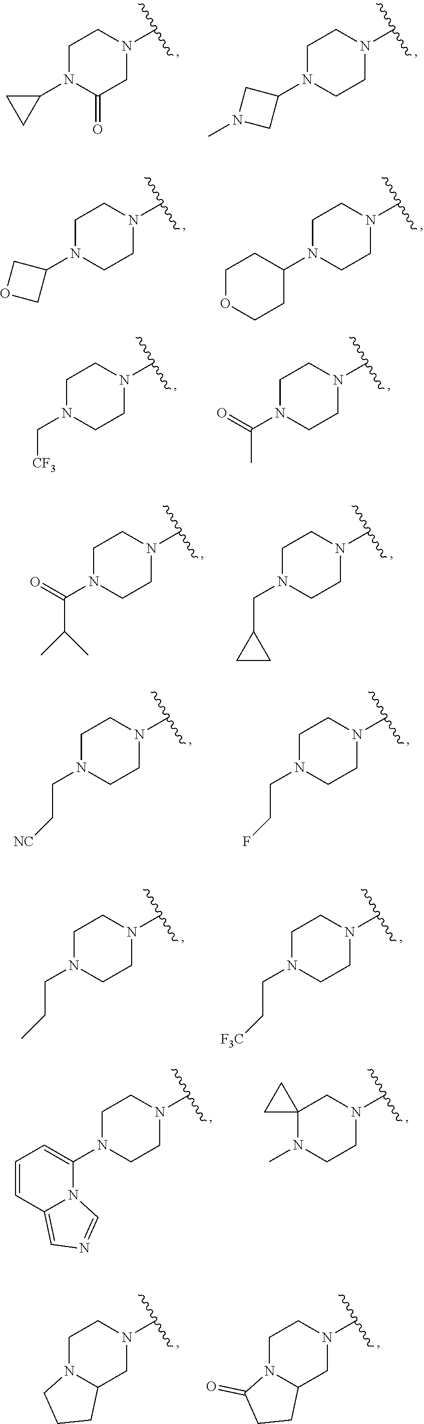

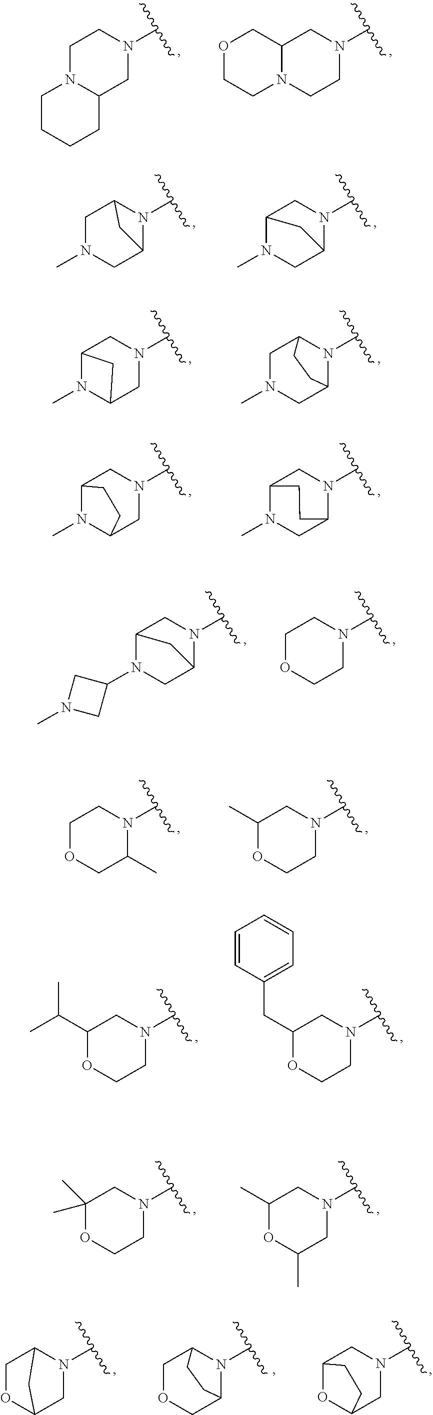

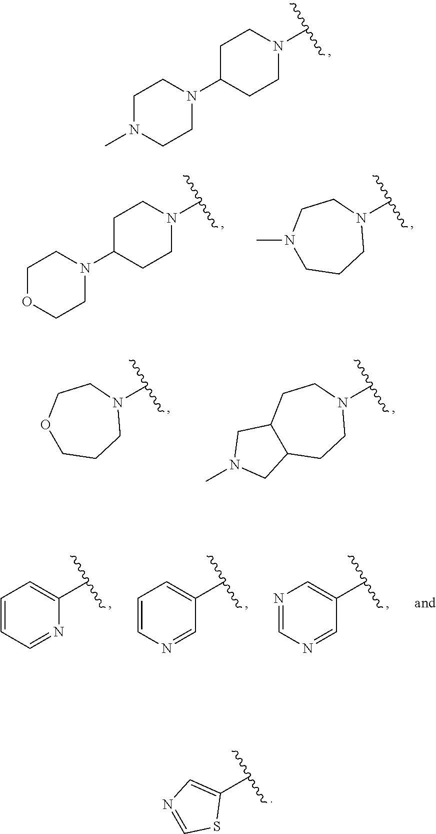

5. The compound of any one of claims 1-4, wherein R¹ is

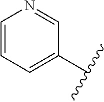 [SMILES: *c1cccnc1]

6. The compound of any one of claims 1-4, wherein R¹ is

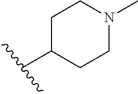 [SMILES: *C1CCN(C)CC1]

7. The compound of any one of claims 1-4, wherein R¹ is hydrogen.

8. The compound of any one of claims 1-7, wherein R² is selected from the group consisting of hydrogen, halogen, C₁₋₄alkyl, C₁₋₄haloalkyl, —O—C₁₋₄alkyl, —O—C₁₋₄haloalkyl, O—C₃₋₇cycloalkyl, —O-(3-7 membered heterocyclyl), and cyano, wherein any aforementioned 3-7 membered heterocyclyl is optionally substituted.

9. The compound of any one of claims 1-8, wherein R² is selected from the group consisting of hydrogen, chlorine, fluorine, —CF₃, methoxy, methyl, cyano, —O—CF₃, —OCH₂CH₂N(CH₃)₂,

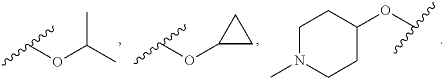

10. The compound of any one of claims 1-9, wherein R² is selected from the group consisting of hydrogen, chlorine, fluorine, —CF₃, methoxy, methyl, cyano,

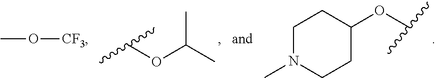

11. The compound of any one of claims 1-10, wherein R² is selected from the group consisting of hydrogen, chlorine, fluorine, —CF₃, methoxy, and methyl.

12. The compound of any one of claims 1-11, wherein R² is hydrogen.

13. The compound of any one of claims 1-11, wherein R² is methoxy.

14. A compound of formula (I-a): 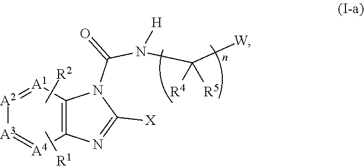
or a pharmaceutically acceptable salt thereof, wherein:
R¹ is selected from the group consisting of phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl);
R² is selected from the group consisting of hydrogen, halogen, C₁₋₆alkyl, C₁₋₆haloalkyl, —OR^{c}, and cyano;
R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene;
n is integer selected from 0 to 6;
X is selected from the group consisting of hydrogen, deuterium, —OR^{b}, —S(C₁₋₆alkyl), C₁₋₆alkyl, and phenyl;
R^{a} is independently, for each occurrence, hydrogen or C₁₋₆alkyl;
R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂;
R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;
W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₄alkynylene, —(C₂₋₆alkynylene)-phenyl, and —(C₂₋₆alkynylene)-C₃₋₇cycloalkyl; and
A¹, A², A³, and A⁴ are CH, or one or two of A¹, A², A³, and A⁴ are N and the others are CH;
wherein any aforementioned phenyl, C₃₋₇cycloalkyl, 3-10 membered heterocyclyl, —(C₂₋₆alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted;
wherein the compound is not a compound of 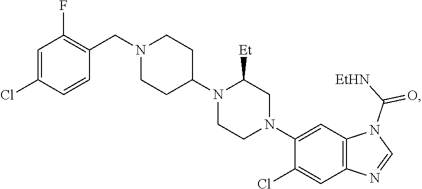
or a pharmaceutically acceptable salt thereof.

15. The compound of claim 14, wherein R¹ is selected from the group consisting of 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl).

16. The compound of claim 14 or 15, wherein R¹ is selected from the group consisting of 3-10 membered heterocyclyl, 5-6 membered heteroaryl, C₁₋₆alkylene-(3-7 membered heterocyclyl), (3-7 membered heterocyclene)-(5-10 membered heteroaryl), (3-7 membered heterocyclene)-(C₃₋₇cycloalkyl), and (3-7 membered heterocyclene)-(3-7 membered heterocyclyl), wherein the 3-10 membered heterocyclyl, (3-7 membered heterocyclene)-(C₃₋₇cycloalkyl), and (3-7 membered heterocyclene)-(3-7 membered heterocyclyl) are optionally substituted with 1-3 substituents independently, for each occurrence, selected from the group consisting of halogen, cyano, oxo, C₁₋₆alkyl, —C₁₋₆alkylene-CN, C₁₋₆haloalkyl, —O—C₁₋₆alkyl, —N(R^{a})₂, —C(O)—C₁₋₆alkyl, and —C₁₋₆alkylene-(C₃₋₇cycloalkyl).

17. The compound of any one of claims 14-16, wherein R¹ is selected from the group consisting of

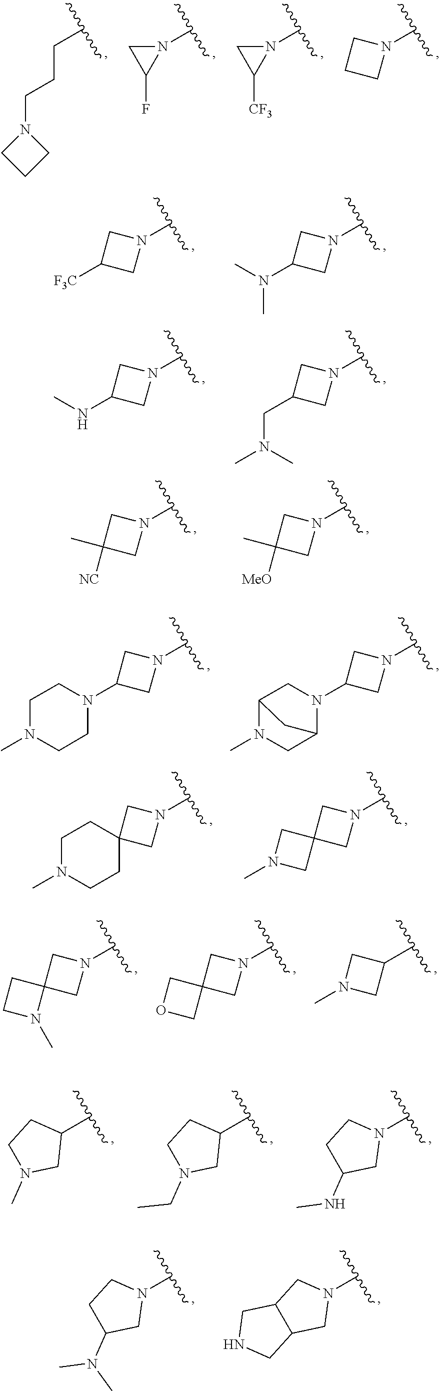

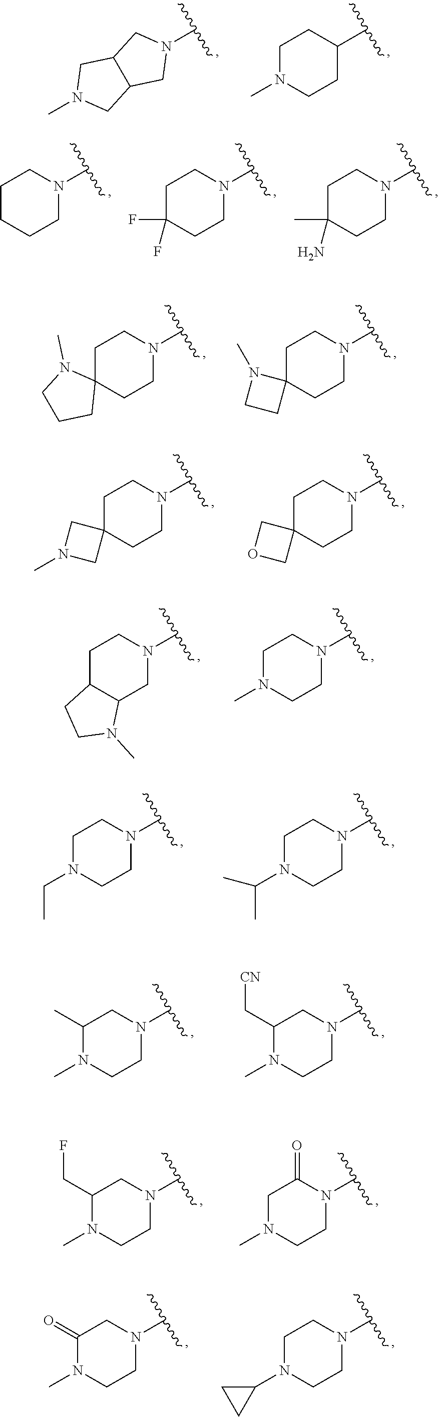

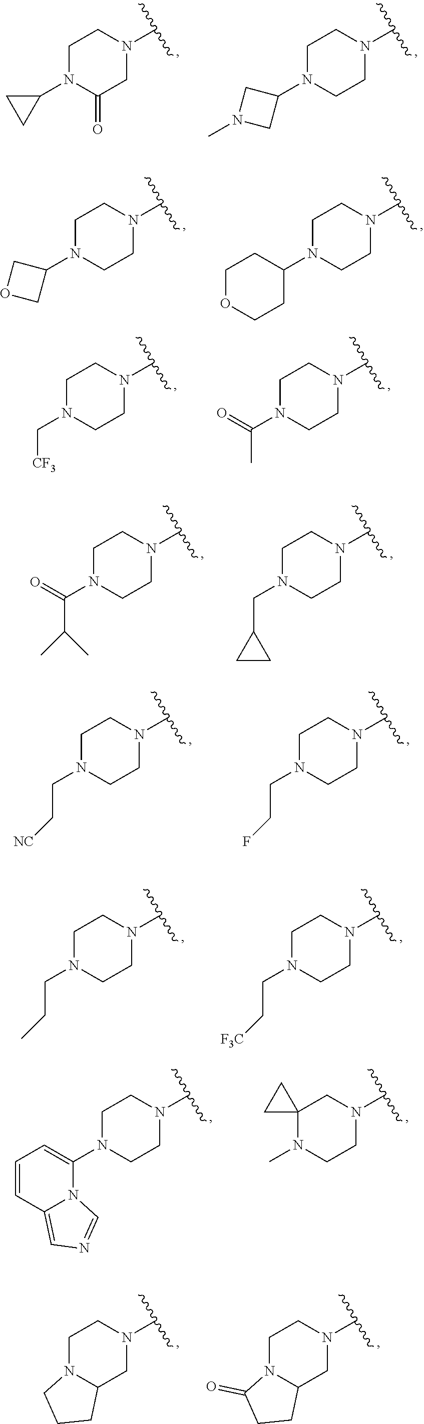

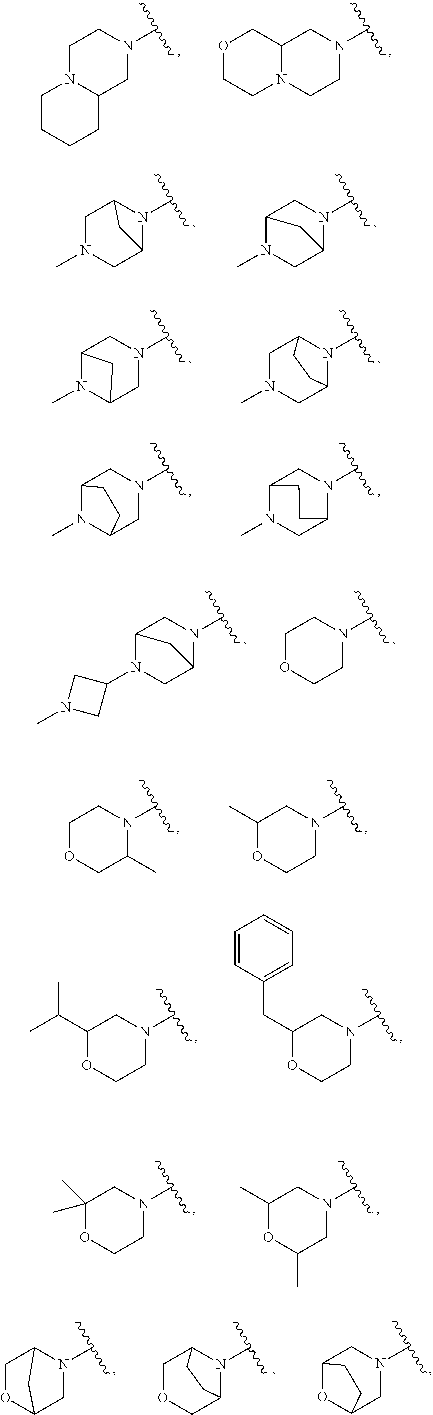

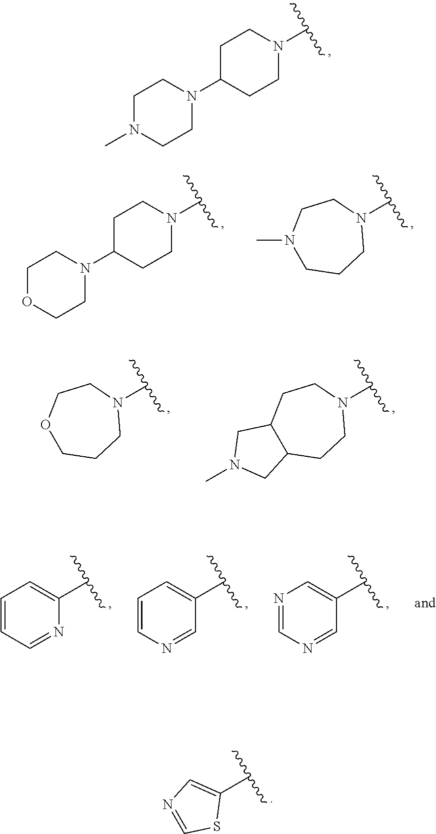

18. The compound of any one of claims 14-17, wherein R¹ is

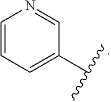 [SMILES: *c1cccnc1]

19. The compound of any one of claims 14-17, wherein R¹ is

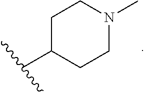 [SMILES: *C1CCN(C)CC1]

20. The compound of any one of claims 14-19, R² is selected from the group consisting of hydrogen, halogen, C₁₋₄alkyl, C₁₋₄haloalkyl, —O—C₁₋₄alkyl, and cyano.

21. The compound of any one of claims 14-20, wherein R² is selected from the group consisting of hydrogen, chlorine, fluorine, —CF₃, methoxy, methyl, and cyano.

22. The compound of any one of claims 14-21, wherein R² is selected from the group consisting of hydrogen, chlorine, fluorine, —CF₃, methoxy, and methyl.

23. The compound of any one of claims 14-22, wherein R² is hydrogen.

24. The compound of any one of claims 14-22, wherein R² is methoxy.

25. A compound of formula (I-b): 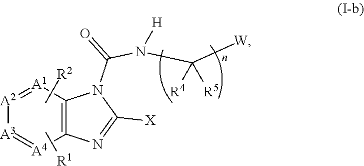
or a pharmaceutically acceptable salt thereof,
wherein:
R² is selected from the group consisting of halogen, C₁₋₆alkyl, C₁₋₆haloalkyl, —OR^{c}, and cyano; and
R¹ is selected from the group consisting of hydrogen, phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl);
R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene;
n is integer selected from 0 to 6;
X is selected from the group consisting of hydrogen, deuterium, —OR^{b}, —S(C₁₋₆alkyl), C₁₋₆ alkyl, and phenyl;
R^{a} is independently, for each occurrence, hydrogen or C₁₋₆alkyl;
R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂;
R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;
W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl; and
A¹, A², A³, and A⁴ are CH, or one or two of A¹, A², A³, and A⁴ are N and the others are CH;
wherein any aforementioned phenyl, C₃₋₇cycloalkyl, 3-10 membered heterocyclyl, —(C₂₋₆alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted;
wherein the compound is not a compound of 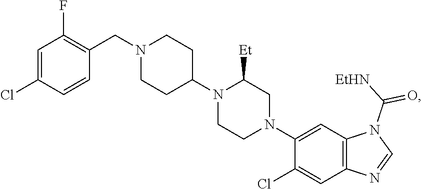
or a pharmaceutically acceptable salt thereof.

26. The compound of claim 25, wherein R¹ is selected from the group consisting of hydrogen, 3-10 membered heterocyclyl, 5-6 membered heteroaryl, and (3-7 membered heterocyclene)-(3-7 membered heterocyclyl).

27. The compound of claim 25 or 26, wherein R¹ is selected from the group consisting of hydrogen, 3-10 membered heterocyclyl, 5-6 membered heteroaryl, and (3-7 membered heterocyclene)-(3-7 membered heterocyclyl), wherein the 3-10 membered heterocyclyl and (3-7 membered heterocyclene)-(3-7 membered heterocyclyl) are optionally substituted with C₁₋₆alkyl.

28. The compound of any one of claims 25-27, wherein R¹ is selected from the group consisting of hydrogen,

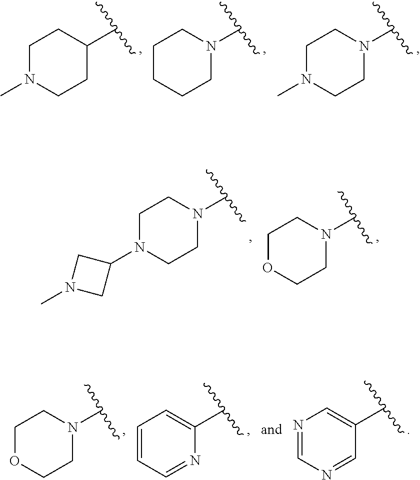

29. The compound of any one of claims 25-28, wherein R¹ is

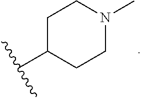 [SMILES: *C1CCN(C)CC1]

30. The compound of any one of claims 25-28, wherein R¹ is hydrogen.

31. The compound of any one of claims 25-30, wherein R² is selected from the group consisting of halogen, C₁₋₄alkyl, C₁₋₄haloalkyl, —O—C₁₋₄alkyl, —O—C₁₋₄haloalkyl, —O-(3-7 membered heterocyclyl), and cyano, wherein any aforementioned 3-7 membered heterocyclyl is optionally substituted.

32. The compound of any one of claims 25-31, wherein R² is selected from the group consisting of chlorine, fluorine, —CF₃, methoxy, methyl, cyano,

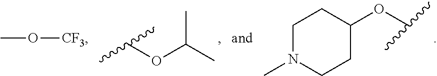

33. The compound of any one of claims 25-32, wherein R² is selected from the group consisting of chlorine, fluorine, —CF₃, methoxy, and methyl.

34. The compound of any one of claims 25-33, wherein R² is methoxy.

35. The compound of any one of claims 1-34, wherein n is 1, 2, 3, or 4.

36. The compound of any one of claim 35, wherein n is 2.

37. The compound of any one of claim 35, wherein n is 4.

38. The compound of any one of claims 1-37, wherein W is selected from the group consisting of methyl, halogen, phenyl, 3-10 membered heterocyclyl, C₃₋₇cycloalkyl, —O—C₁₋₄alkyl, —O—C₁₋₄ haloalkyl, —O-phenyl, —O—(C₁₋₄alkylene)-phenyl, 5-6 membered heteroaryl, C₂₋₆alkynylene, —(C₂₋₆alkynylene)-phenyl, and —(C₂₋₆alkynylene)-C₃₋₇cycloalkyl, wherein the phenyl, —O-phenyl, 5-6 membered heteroaryl, C₃₋₇cycloalkyl, C₂₋₆alkynylene, —(C₂₋₆alkynylene)-phenyl, —(C₂₋₆alkynylene)-C₃₋₇cycloalkyl, and 3-10 membered heterocyclyl, wherein the phenyl, C₃₋₇cycloalkyl, 5-6 membered heteroaryl, C₂₋₆alkynylene, —(C₂₋₆alkynylene)-phenyl, and —(C₂₋₆alkynylene)-C₃₋₇cycloalkyl are optionally substituted with 1-3 substituents independently, for each occurrence, selected from the group consisting of fluorine, C₁₋₆alkyl, C₁₋₆haloalkyl, —O—C₁₋₆alkyl, —C₁₋₆alkylene-phenyl, and C₂₋₆alkynylene.

39. The compound of any one of claims 1-38, wherein W is selected from the group consisting of methyl, fluorine, methoxy, —O—CF₃, phenyl, —O-phenyl,

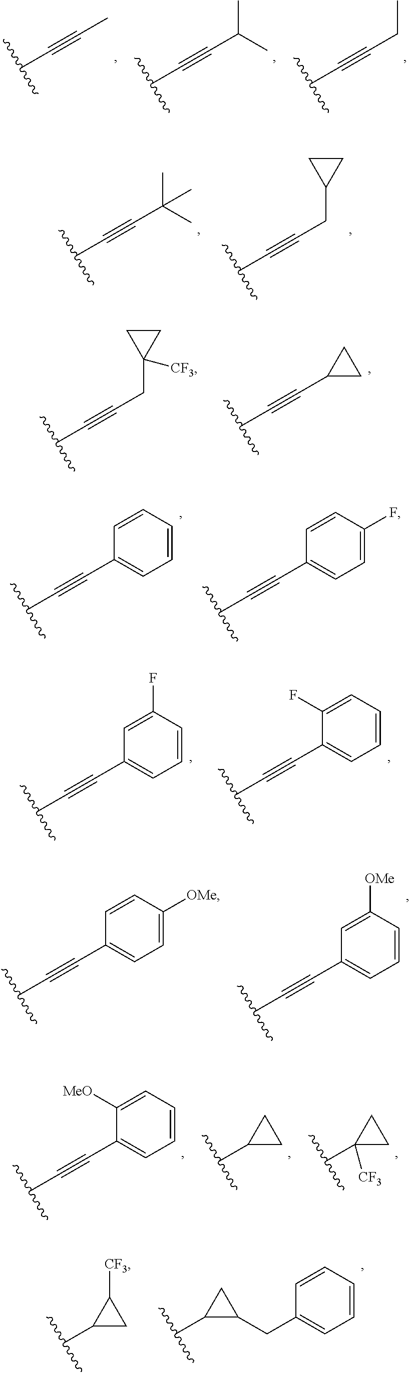

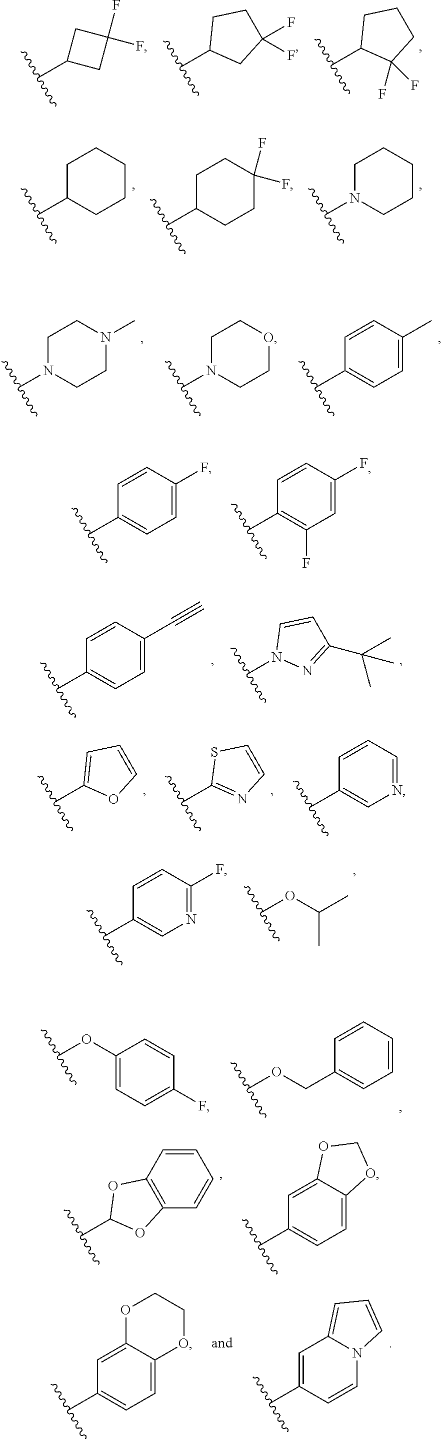

40. The compound of any one of claims 1-39 wherein W is selected from the group consisting of methyl, fluorine, —O—CF₃, phenyl, —O-phenyl,

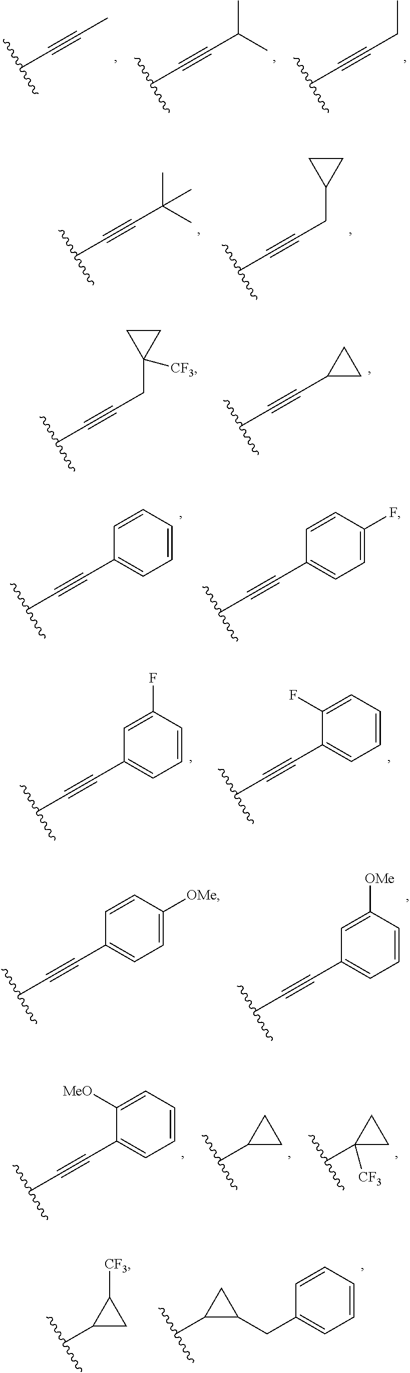

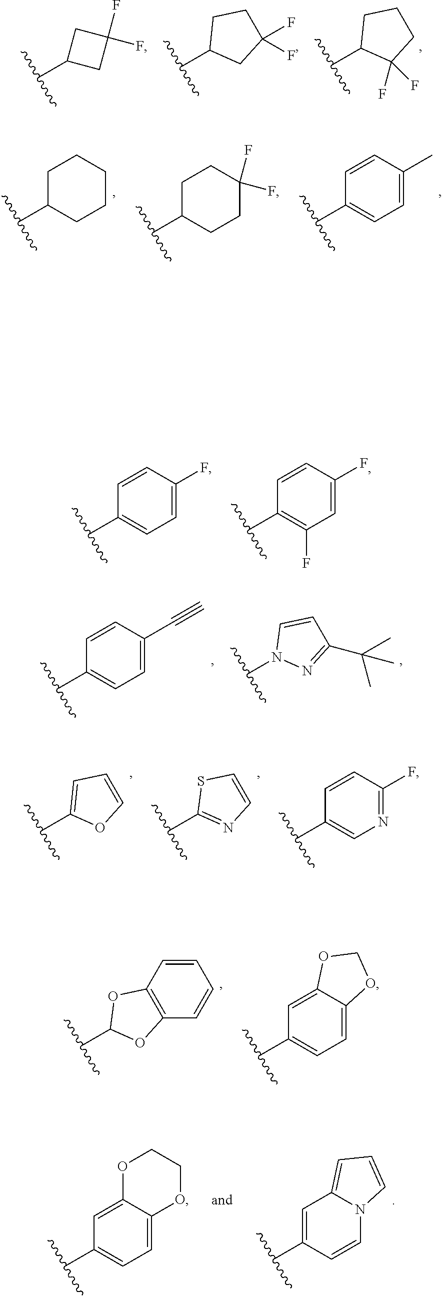

41. The compound of any one of claims 1-39, wherein W is selected from the group consisting of methyl, phenyl, —O-phenyl,

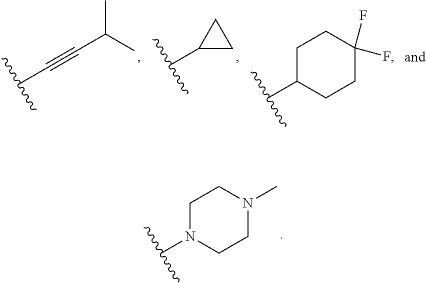

42. The compound of any one of claims 1-41, wherein W is selected from the group consisting of methyl, phenyl, and —O-phenyl.

43. The compound of any one of claims 1-42, wherein W is selected from methyl and phenyl.

44. The compound of any one of claims 1-43, wherein W is phenyl.

45. The compound of any one of claims 1-43, wherein W is methyl.

46. A compound of formula (I-c): 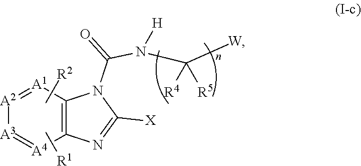
or a pharmaceutically acceptable salt thereof,
wherein:
W is phenyl; and n is integer selected from 0, 2, 3, 5, or 6, or
W is methyl; and n is integer selected from 0, 1, 2, 3, 4, or 6, or
W is selected from the group consisting of halogen, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆ alkylene)-phenyl, C₂₋₆alkynylene, —(C₂₋₆alkynylene)-phenyl, and —(C₂₋₆alkynylene)-C₃₋₇cycloalkyl; and n is integer selected from 0, 1, 2, 3, 4, 5, or 6, and
R¹ is selected from the group consisting of hydrogen, phenyl, C₃₋₇cycloalkyl, 3-10 membered heterocyclyl, 5-6 membered heteroaryl, C₁₋₆alkylene-N(R^{a})₂, C₁₋₆alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl);
R² is selected from the group consisting of hydrogen, halogen, C₁₋₆alkyl, C₁₋₆haloalkyl, —OR^{c}, and cyano;
R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene;
X is selected from the group consisting of hydrogen, deuterium, —OR^{b}, —S(C₁₋₆alkyl), C₁₋₆ alkyl, and phenyl;
R^{a} is independently, for each occurrence, hydrogen or C₁₋₆alkyl;
R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, and C₁₋₆alkylene-OR^{a}, C₁₋₆alkylene-N(R^{a})₂;
R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;
A¹, A², A³, and A⁴ are CH, or one or two of A¹, A², A³, and A⁴ are N and the others are CH; and
wherein any aforementioned phenyl, C₃₋₇cycloalkyl, 3-10 membered heterocyclyl, —(C₂₋₆ alkynylene)-phenyl, —(C₂₋₆alkynylene)-C₃₋₇cycloalkyl, (3-7 membered heterocyclene)-(3-7 membered heterocyclyl), or 5-6 membered heteroaryl is optionally substituted.

47. The compound of claim 46, wherein R¹ is selected from the group consisting of hydrogen, 3-10 membered heterocyclyl, 5-6 membered heteroaryl, C₁₋₆alkylene-N(R^{a})₂, C₁₋₄alkylene-(3-7 membered heterocyclyl), (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), (5-6 membered heteroarylene)-(3-7 membered heterocyclyl) and —C(O)-(3-7 membered heterocyclyl).

48. The compound of claim 46 or 47, wherein R¹ is selected from the group consisting of hydrogen, 3-10 membered heterocyclyl, 5-6 membered heteroaryl, C₁₋₆alkylene-(3-7 membered heterocyclyl), (3-7 membered heterocyclene)-(5-10 membered heteroaryl), (3-7 membered heterocyclene)-(C₃₋₇cycloalkyl), and (3-7 membered heterocyclene)-(3-7 membered heterocyclyl), wherein the 3-10 membered heterocyclyl, (3-7 membered heterocyclene)-(C₃₋₇cycloalkyl), and (3-7 membered heterocyclene)-(3-7 membered heterocyclyl) are optionally substituted with 1-3 substituents independently, for each occurrence, selected from the group consisting of halogen, cyano, oxo, C₁₋₆alkyl, —C₁₋₆alkylene-CN, C₁₋₆haloalkyl, —O—C₁₋₆alkyl, —N(R^{a})₂, —C(O)—C₁₋₆alkyl, and —C₁₋₆alkylene-(C₃₋₇cycloalkyl).

49. The compound of any one of claims 46-48, wherein R¹ is selected from the group consisting of hydrogen,

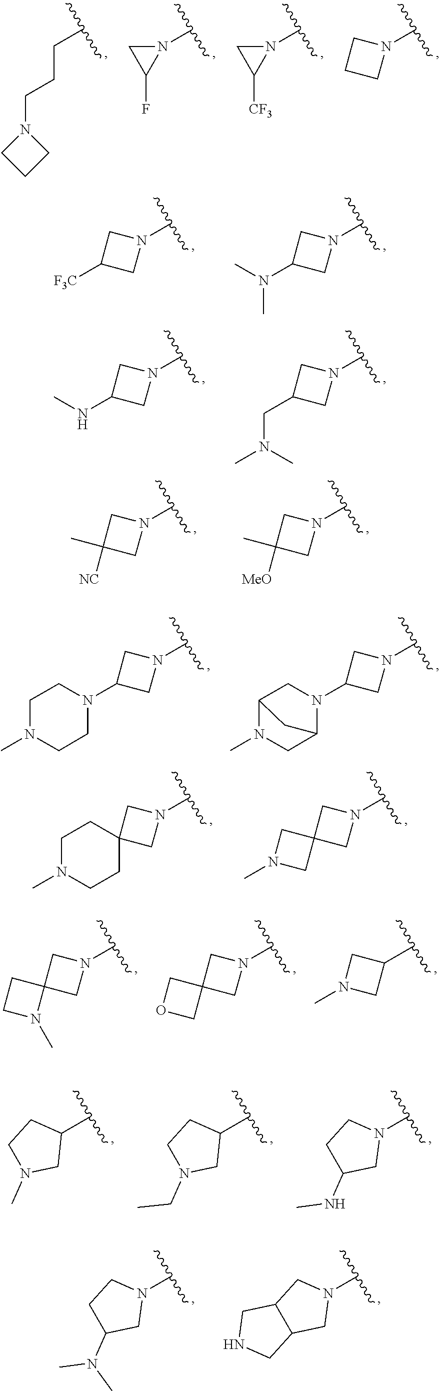

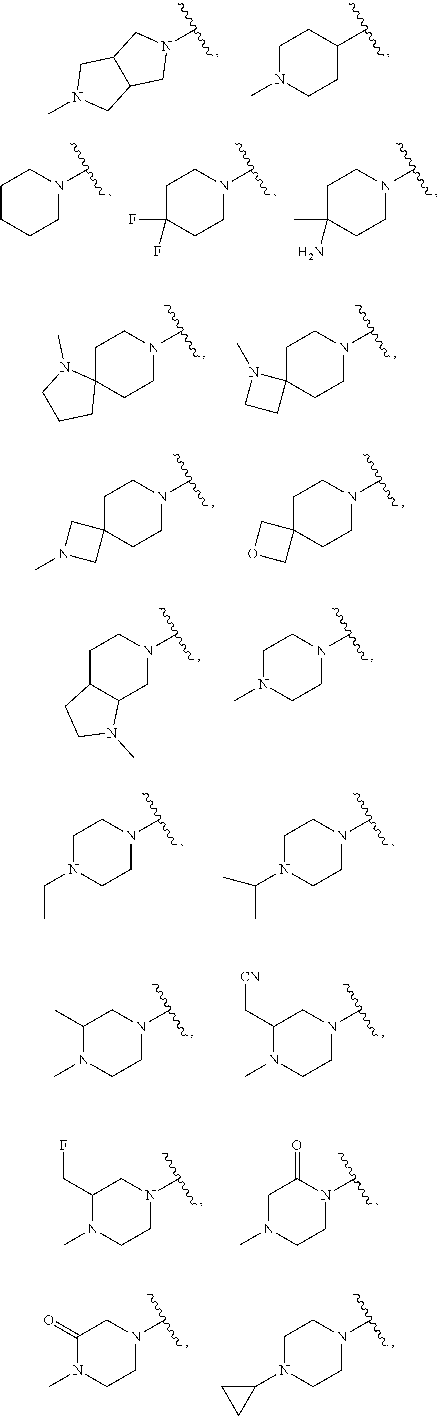

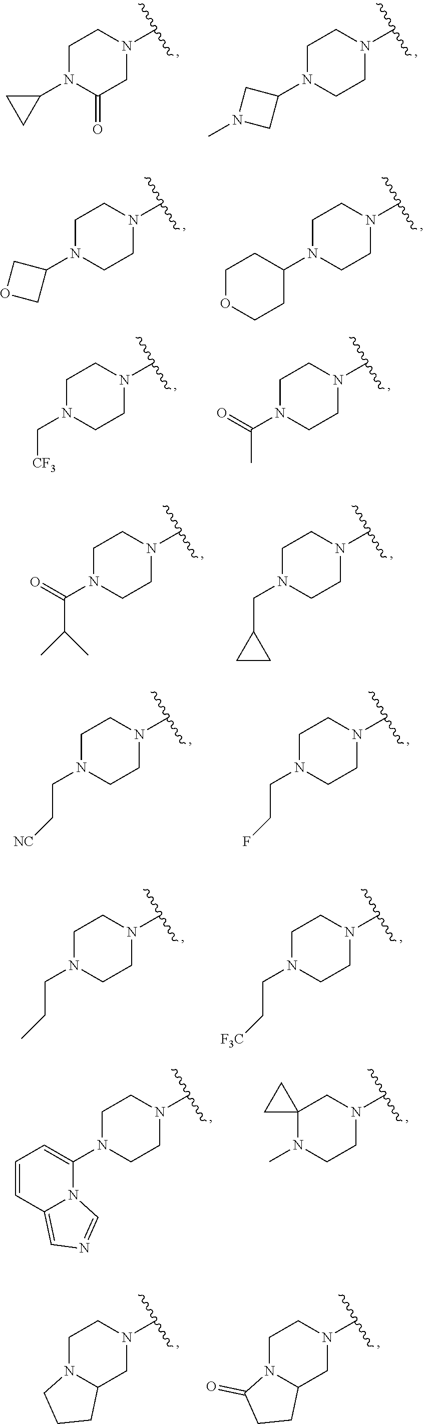

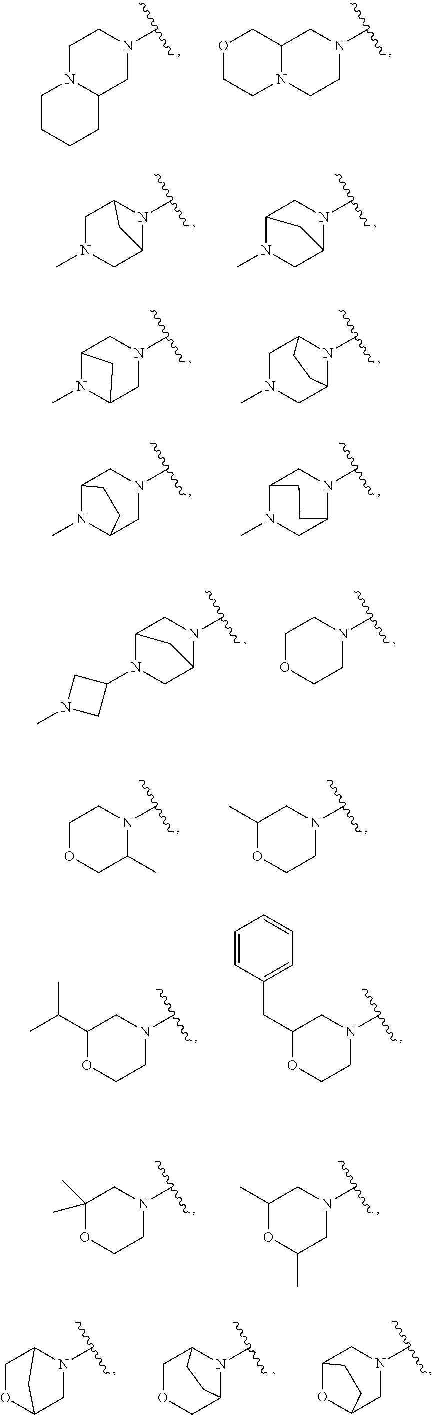

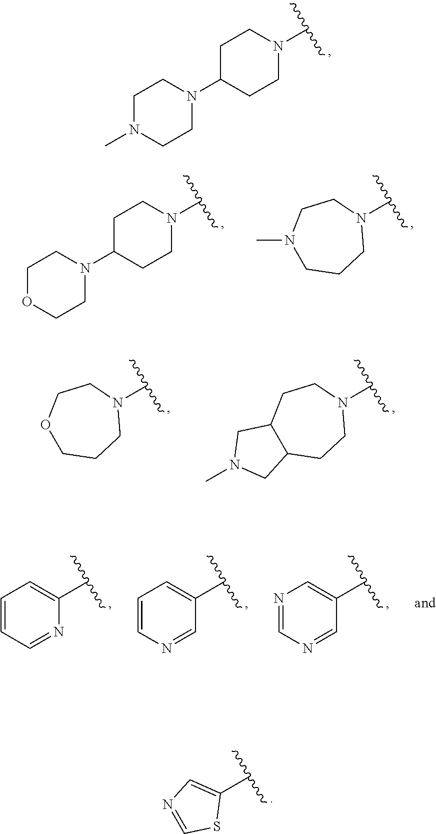

50. The compound ofany one of claims 46-49, wherein R¹ is

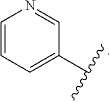 [SMILES: *c1cccnc1]

51. The compound of any one of claims 46-49, wherein R¹ is

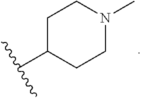 [SMILES: *C1CCN(C)CC1]

52. The compound of any one of claims 46-49, wherein R is hydrogen.

53. The compound of any one of claims 46-52, wherein R² is selected from the group consisting of hydrogen, halogen, C₁₋₄alkyl, C₁₋₄haloalkyl, —O—C₁₋₄alkyl, —O—C₁₋₄haloalkyl, O—C₃₋₇ cycloalkyl, —O-(3-7 membered heterocyclyl), and cyano, wherein any aforementioned 3-7 membered heterocyclyl is optionally substituted.

54. The compound of any one of claims 46-53, wherein R² is selected from the group consisting of hydrogen, chlorine, fluorine, —CF₃, methoxy, methyl, cyano,

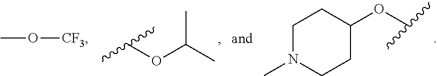

55. The compound of any one of claims 46-54, wherein R² is selected from the group consisting of hydrogen, chlorine, fluorine, —CF₃, methoxy, and methyl.

56. The compound of any one of claims 46-55, wherein R² is hydrogen.

57. The compound of any one of claims 46-55, wherein R² is methoxy.

58. The compound of any one of claims 46-57, wherein n is 0, 1, 2, 3, or 5 and W is selected from the group consisting of methyl, halogen, phenyl, 3-10 membered heterocyclyl, C₃₋₇cycloalkyl, —O—C₁₋₄alkyl, —O—C₁₋₄haloalkyl, —O-phenyl, —O—(C₁₋₄alkylene)-phenyl, 5-6 membered heteroaryl, C₂₋₆alkynylene, —(C₂₋₆alkynylene)-phenyl, and —(C₂₋₆alkynylene)-C₃₋₇cycloalkyl, wherein the phenyl, —O-phenyl, 5-6 membered heteroaryl, C₃₋₇cycloalkyl, C₂₋₆alkynylene, —(C₂₋₆alkynylene)-phenyl, —(C₂₋₆alkynylene)-C₃₋₇cycloalkyl, and 3-10 membered heterocyclyl, wherein the phenyl, C₃₋₇cycloalkyl, 5-6 membered heteroaryl, C₂₋₆alkynylene, —(C₂₋₆alkynylene)-phenyl, and —(C₂₋₆alkynylene)-C₃₋₇cycloalkyl are optionally substituted with 1-3 substituents independently, for each occurrence, selected from the group consisting of fluorine, C₁₋₆alkyl, C₁₋₆haloalkyl, —O—C₁₋₆alkyl, —C₁₋₆alkylene-phenyl, and C₂₋₆alkynylene.

59. The compound of any one of claims 46-58, wherein n is 0, 1, 2, 3, or 5 and W is selected from the group consisting of methyl, fluorine, methoxy, —O—CF₃, phenyl, —O-phenyl,

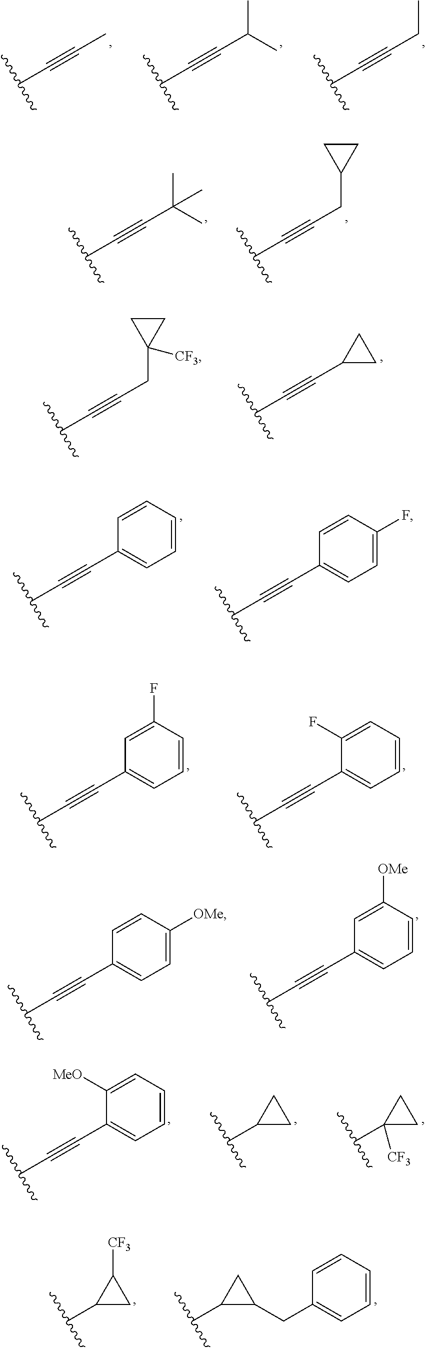

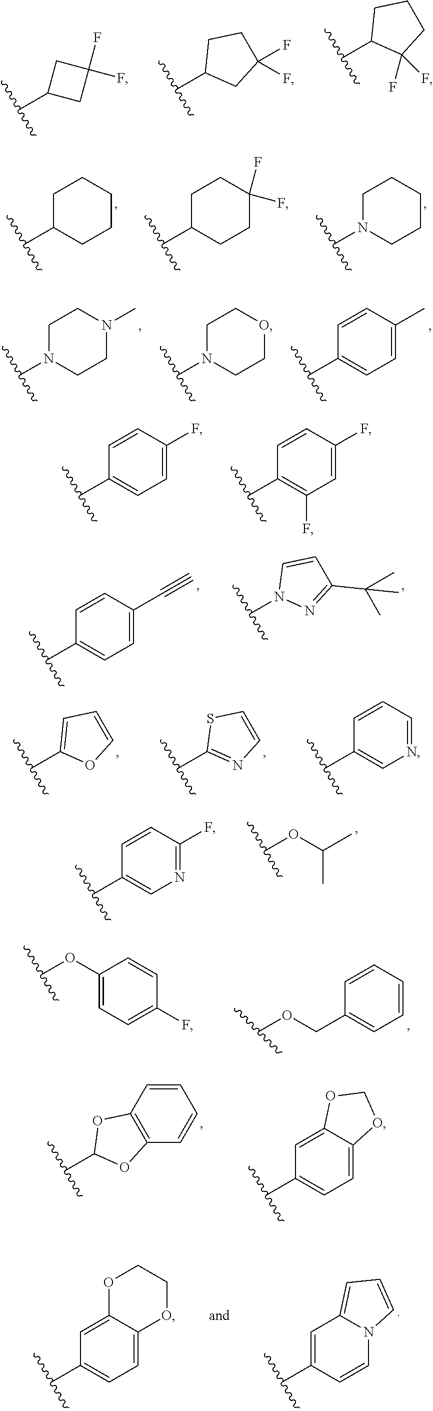

60. The compound of any one of claims 46-59, wherein n is 0, 1, 2, 3, or 5 and W is selected from the group consisting of methyl, phenyl, and —O-phenyl.

61. The compound of any one of claims 46-60, wherein n is 0, 1, 2, 3, or 5 and W is selected from methyl and phenyl.

62. The compound of any one of claims 46-61, wherein n is 0, 1, 2, 3, or 5 and W is phenyl.

63. The compound of any one of claims 46-61, wherein n is 0, 1, 2, 3, or 5 and W is methyl.

64. The compound of any one of claims 46-57, wherein n is 4 and W is selected from the group consisting of methyl, halogen, 3-10 membered heterocyclyl, C₃₋₇cycloalkyl, —O—C₁₋₄alkyl, —O—C₁₋₄haloalkyl, —O-phenyl, —O—(C₁₋₄alkylene)-phenyl, 5-6 membered heteroaryl, C₂₋₆alkynylene, —(C₂₋₆alkynylene)-phenyl, and —(C₂₋₆alkynylene)-C₃₋₇ cycloalkyl, wherein the phenyl, —O-phenyl, 5-6 membered heteroaryl, C₃₋₇cycloalkyl, C₂₋₆alkynylene, —(C₂₋₆alkynylene)-phenyl, —(C₂₋₆alkynylene)-C₃₋₇cycloalkyl, and 3-10 membered heterocyclyl, wherein the phenyl, C₃₋₇ cycloalkyl, 5-6 membered heteroaryl, C₂₋₆alkynylene, —(C₂₋₆alkynylene)-phenyl, and —(C₂₋₆alkynylene)-C₃₋₇cycloalkyl are optionally substituted with 1-3 substituents independently, for each occurrence, selected from the group consisting of fluorine, C₁₋₆alkyl, C₁₋₆haloalkyl, —O—C₁₋₆alkyl, —C₁₋₆alkylene-phenyl, and C₂₋₆alkynylene.

65. The compound of any one of claims 46-57, and 64, wherein n is 4 and W is selected from the group consisting of methyl,

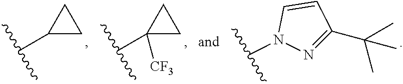

66. The compound of any one of claims 46-57, 64 and 65, wherein n is 4 and W is methyl or —O-phenyl.

67. The compound of any one of claims 46-57 and 64-66, wherein n is 4 and W is methyl.

68. The compound of any one of claims 1-67, wherein X is selected from the group consisting of hydrogen, deuterium, methyl, OR^{b}, and —SCH₃.

69. The compound of any one of claims 1-68, wherein X is selected from the group consisting of hydrogen, deuterium, methyl, methoxy,

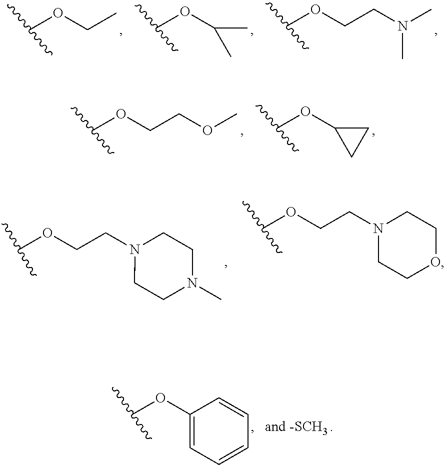

70. The compound of claim 69, wherein X is

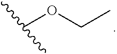 [SMILES: *OCC]

71. The compound of any one of claims 1-70, wherein R⁴ and R⁵ are independently, for each occurrence, selected from the group consisting of hydrogen, methyl, fluorine, and CF₃; or R⁴ and R⁵ can be taken together to form cyclopropyl.

72. The compound of any one of claims 1-70, wherein R⁴ and R⁵ are independently selected from hydrogen and methyl.

73. The compound of any one of claims 1-70, wherein R⁴ and R⁵ are hydrogen.

74. The compound of any one of claims 1-73, wherein A¹, A², A³, and A⁴ are CH.

75. The compound of any one of claims 1-73, wherein A¹, A², and A³ are CH and A⁴ is N.

76. The compound of any one of claims 1-73, wherein A¹, A², and A⁴ are CH and A³ is N.

77. The compound of any one of claims 1-73, wherein A¹, A³, and A⁴ are CH and A² is N.

78. The compound of any one of claims 1-73, wherein A², A³, and A⁴ are CH and A¹ is N.

79. The compound of claim 1, wherein the compound is a compound of formula (I-d): 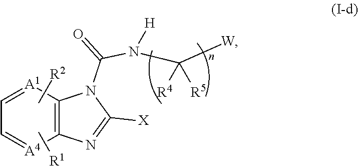
or a pharmaceutically acceptable salt thereof,
wherein:
R¹, R², R⁴, R⁵, A¹, A⁴, X, n, and W are as defined in claim 1.

80. The compound of claim 1, wherein the compound is a compound of formula (I-e): 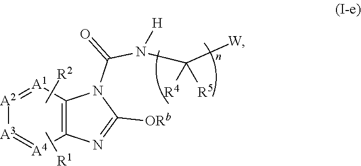
or a pharmaceutically acceptable salt thereof, wherein the variables are as defined in claim 1.

81. The compound of claim 1, wherein the compound is a compound of formula (I-f): 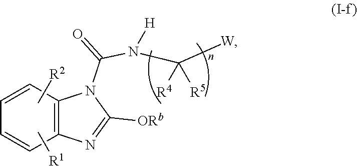
or a pharmaceutically acceptable salt thereof, wherein the variables are as defined in claim 1.

82. The compound of claim 1, wherein the compound is a compound of formula (I-g): 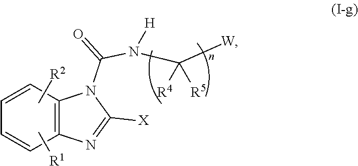
or a pharmaceutically acceptable salt thereof, wherein:
R¹ is selected from the group consisting of phenyl, C₃₋₇cycloalkyl, 3-10 membered heterocyclyl, 5-6 membered heteroaryl, (3-7 membered heterocyclene)-(5-10 membered heteroaryl), (3-7 membered heterocyclene)-(C₃₋₇cycloalkyl), (3-7 membered heterocyclene)-(3-7 membered heterocyclyl), and (5-6 membered heteroarylene)-(3-7 membered heterocyclyl), wherein the phenyl, C₃₋₇cycloalkyl, 3-10 membered heterocyclyl, 5-6 membered heteroaryl, or (3-7 membered heterocyclene)-(3-7 membered heterocyclyl) is optionally substituted with one or more substituents independently, for each occurrence, selected from the group consisting of halogen, cyano, oxo, C₁₋₆alkyl, —C₁₋₆alkylene-CN, C₁₋₆haloalkyl, —O—C₁₋₆alkyl, —N(R^{a})₂, —C(O)—C₁₋₆ alkyl, —C₁₋₆alkylene-unsubstituted phenyl, —C₁₋₆alkylene-N(R^{a})₂, and —C₁₋₆alkylene-(C₃₋₇cycloalkyl);
R² is selected from the group consisting of hydrogen, halogen, C₁₋₆alkyl, C₁₋₆haloalkyl, —OR^{c}, and cyano;
R⁴ and R⁵ are independently, for each occurrence, selected from the group consisting of hydrogen, C₁₋₆alkyl, C₁₋₆haloalkyl, and halogen; or R⁴ and R⁵ can be taken together to form C₃₋₇cycloalkylene, wherein the C₃₋₇cycloalkylene is optionally substituted;
n is integer selected from 0 to 6;
X is selected from the group consisting of hydrogen, deuterium, —OR^{b}, —S(C₁₋₆alkyl), C₁₋₆alkyl, and phenyl, wherein the phenyl is optionally substituted;
R^{a} is independently, for each occurrence, hydrogen or C₁₋₆alkyl;
R^{b} is independently, for each occurrence, selected from the group consisting of C₁₋₆alkyl, C₁₋₆haloalkyl, C₃₋₇cycloalkyl, 3-7 membered heterocyclyl, C₁₋₆alkylene-(3-7 membered heterocyclyl), 5-6 membered heteroaryl, phenyl, C₁₋₆alkylene-OR^{a}, and C₁₋₆alkylene-N(R^{a})₂, wherein the phenyl, C₃₋₇cycloalkyl, 3-7 membered heterocyclyl, or 5-6 membered heteroaryl are optionally substituted;
R^{c} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂, wherein the phenyl, C₃₋₇cycloalkyl, 3-7 membered heterocyclyl, or 5-6 membered heteroaryl are optionally substituted; and
W is selected from the group consisting of methyl, halogen, phenyl, C₃₋₇cycloalkyl, 3-10 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl, wherein the phenyl, C₃₋₇cycloalkyl, 3-10 membered heterocyclyl, —(C₂₋₆alkynylene)-phenyl, —(C₂₋₆alkynylene)-C₃₋₇cycloalkyl, or 5-6 membered heteroaryl is optionally substituted.

83. The compound of claim 82, wherein the compound is a compound of formula (I-h): 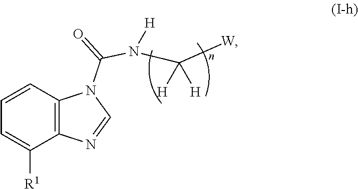
or a pharmaceutically acceptable salt thereof, wherein:
R¹ is 3-10 membered monocyclic or bicyclic heterocyclyl or (3-7 membered heterocyclene)-(3-7 membered heterocyclyl), wherein the 3-10 membered monocyclic or bicyclic heterocyclyl or (3-7 membered heterocyclene)-(3-7 membered heterocyclyl) is optionally substituted with C₁₋₆alkyl;
n is 2 or 3; and
W is phenyl, —O-phenyl, or —(C₂₋₆alkynylene)-phenyl.

84. The compound of claim 82 or 83, wherein R¹ is selected from the group consisting of

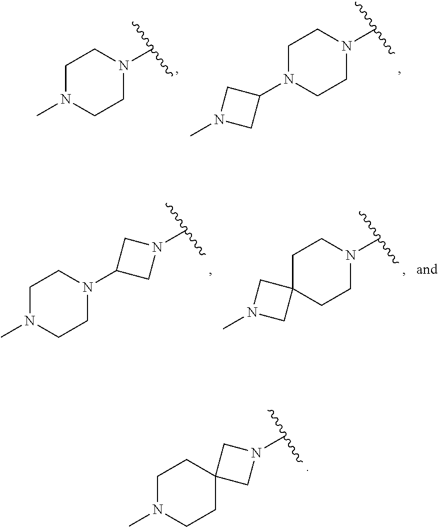

85. The compound of any one of claims 82-84, wherein n is 2.

86. The compound of any one of claims 82-84, wherein n is 3.

87. The compound of any one of claims 82-86, wherein W is selected from the group consisting of phenyl, —O-phenyl, and

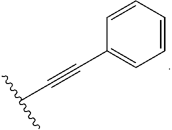 [SMILES: *C#Cc1ccccc1]

88. A compound of formula (II): 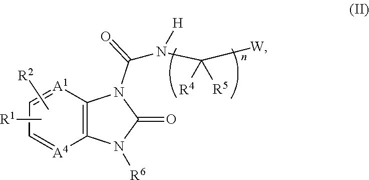
or a pharmaceutically acceptable salt thereof, wherein:
A¹ and A⁴ are independently selected from CH and N;
R¹ is selected from the group consisting of hydrogen, phenyl, C₃₋₇cycloalkyl, 3-7 membered heterocyclyl, 5-6 membered heteroaryl, C₁₋₆alkylene-N(R^{a})₂, (3-7 membered heterocyclene)-(3-7 membered heterocyclyl), (5-6 membered heteroarylene)-(3-7 membered heterocyclyl) and —C(O)-(3-7 membered heterocyclyl);
R² is selected from the group consisting of hydrogen, halogen, C₁₋₆alkyl, C₁₋₆haloalkyl, —OR^{c}, and cyano;
R⁴ and R⁵ are independently, for each occurrence, selected from the group consisting of hydrogen, C₁₋₆alkyl, C₁₋₆haloalkyl, and halogen;
R⁶ is hydrogen or C₁₋₂ alkyl;
n is integer between 0 to 6;
R^{a} is independently hydrogen or C₁₋₆alkyl;
R^{b} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;
R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; and
W is selected from the group consisting of methyl, halogen, phenyl, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, and —(C₂₋₆alkynylene)-C₃₋₇cycloalkyl,
wherein any aforementioned phenyl, 3-7 membered heterocyclyl, or 5-6 membered heteroaryl is optionally substituted,
wherein, the compound is not a compound of 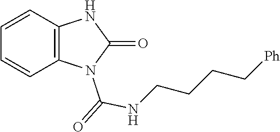 [SMILES: O=C(NCCCCc1ccccc1)n1c(=O)[nH]c2ccccc21]
H or a pharmaceutically acceptable salt thereof.

89. A compound of formula (II-a): 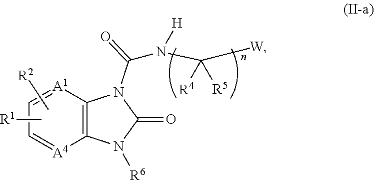
or a pharmaceutically acceptable salt thereof, wherein:
A¹ and A⁴ are independently selected from CH and N;
R¹ is selected from the group consisting of hydrogen, phenyl, C₃₋₇cycloalkyl, 3-7 membered heterocyclyl, 5-6 membered heteroaryl, C₁₋₆alkylene-N(R^{a})₂, (3-7 membered heterocyclene)-(3-7 membered heterocyclyl), (5-6 membered heteroarylene)-(3-7 membered heterocyclyl) and —C(O)-(3-7 membered heterocyclyl);
R² is selected from the group consisting of hydrogen, halogen, C₁₋₆alkyl, C₁₋₆haloalkyl, —OR^{c}, and cyano;
R⁴ and R⁵ are independently, for each occurrence, selected from the group consisting of hydrogen, C₁₋₆alkyl, C₁₋₆haloalkyl, and halogen;
R⁶ is hydrogen or C₁₋₂ alkyl;
R^{a} is independently hydrogen or C₁₋₆alkyl;
R^{b} is independently, for each occurrence, selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;
R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; and
W is selected from the group consisting of methyl, halogen, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, and —(C₂₋₆alkynylene)-C₃₋₇cycloalkyl, and n is integer selected from 0, 1, 2, 3, 4, 5, or 6, or
W is phenyl, and n is integer selected from 0, 1, 2, 3, 5, or 6,
wherein any aforementioned phenyl, 3-7 membered heterocyclyl, or 5-6 membered heteroaryl is optionally substituted.

90. The compound of claim 88 or 89, wherein A¹ and A⁴ are CH.

91. The compound of claim 88 or 89, wherein A¹ is N and A⁴ is CH.

92. The compound of claim 88 or 89, wherein A¹ is CH and A⁴ is N.

93. The compound of any one of claims 88-92, wherein R¹ is 5-6 membered heteroaryl or hydrogen.

94. The compound of any one of claims 88-93, wherein R¹ is selected from the group consisting of hydrogen,

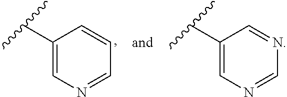 [SMILES: *c1cccnc1 ; *c1cncnc1]

95. The compound of any one of claims 88-94, wherein R¹ is hydrogen.

96. The compound of any one of claims 88-95, wherein R² is hydrogen.

97. The compound of any one of claims 88-96, wherein R⁴ and R are independently selected, at each occurrence, from hydrogen and methyl.

98. The compound of any one of claims 88-97, wherein R⁴ and R⁵ are hydrogen.

99. The compound of any one of claims 88-98, wherein R⁶ is selected from the group consisting of hydrogen, methyl, and ethyl.

100. The compound of any one of claims 88-99, wherein n is 2.

101. The compound of any one of claims 88-99, wherein n is 3.

102. The compound of any one of claims 88-99, wherein n is 4.

103. The compound of any one of claims 88-102, wherein W is selected from the group consisting of methyl, phenyl, and 5-6 membered heteroaryl, wherein the 5-6 membered heteroaryl is optionally substituted.

104. The compound of any one of claims 88-103, wherein W is selected from the group consisting of methyl, phenyl, and

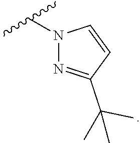 [SMILES: *n1ccc(C(C)(C)C)n1]

105. A pharmaceutical composition comprising the compound of any one of claims 1-104 and a pharmaceutically acceptable carrier.

106. A method of treating a subject with cancer and in need thereof, the method comprising administering to the subject a therapeutically effective amount of the compound of any one of claims 1-104 or a pharmaceutical composition of claim 105.

107. The method of claim 106, wherein the cancer is glioblastoma.

108. A method of treating a subject with a lysosomal storage disorder and in need thereof, the method comprising administering to the subject a therapeutically effective amount of the compound of any one of claims 1-104 or a pharmaceutical composition of claim 105.

109. The method of claim 108, wherein the lysosomal storage disorder is selected from the group consisting of: Krabbe disease, Fabry disease, Tay-Sachs disease, Pompe disease, Hunter's syndrome, Niemann Pick disease Types A and B, and Gaucher disease.

110. The method of claim 109, wherein the lysosomal storage disorder is Fabry disease.

111. A method of treating a subject with a neurodegenerative disorder and in need thereof, the method comprising administering to the subject a therapeutically effective amount of the compound of any one of claims 1-104 or a pharmaceutical composition of claim 105.

112. The method of claim 111, wherein the neurodegenerative disorder is selected from the group consisting of: Alzheimer's disease, Parkinson's disease, Huntington's disease, amyotrophic lateral sclerosis, Lewy body disease, dementia, and multiple system atrophy.

113. The method of claim 112, wherein the neurodegenerative disorder is Parkinson's disease.

114. The method of claim 112, wherein the neurodegenerative disorder is Lewy body disease.

115. The method of claim 112, wherein the neurodegenerative disorder is dementia.

116. The method of claim 112, wherein the neurodegenerative disorder is multiple system atrophy.

117. A method of treating a subject with an inflammatory disorder and in need thereof, the method comprising administering to the subject a therapeutically effective amount of the compound of any one of claims 1-104 or a pharmaceutical composition of claim 105.

118. The method of any one of claims 106-117, wherein the subject is human.

119. A compound of any one of claims 1-104 or a pharmaceutical composition of claim 105 for use in a method of treating a subject with cancer and in need thereof, the method comprising administering to the subject a therapeutically effective amount of the compound or the pharmaceutical composition.

120. A compound of any one of claims 1-104 or a pharmaceutical composition of claim 105 for use in a method of treating a subject with a lysosomal storage disorder and in need thereof, the method comprising administering to the subject a therapeutically effective amount of the compound or the pharmaceutical composition.

121. A compound of any one of claims 1-104 or a pharmaceutical composition of claim 105 for use in a method of treating a subject with a neurodegenerative disorder and in need thereof, the method comprising administering to the subject a therapeutically effective amount of the compound or the pharmaceutical composition.

122. A compound of any one of claims 1-104 or a pharmaceutical composition of claim 105 for use in a method of treating a subject with an inflammatory disorder and in need thereof, the method comprising administering to the subject a therapeutically effective amount of the compound or the pharmaceutical composition.

123. A compound of any one of claims 1-104 or a pharmaceutical composition of claim 105 for the manufacture of a medicament for treating a subject with cancer and in need thereof, the method comprising administering to the subject a therapeutically effective amount of the compound or the pharmaceutical composition.

124. A compound of any one of claims 1-104 or a pharmaceutical composition of claim 105 for the manufacture of a medicament for treating a subject with a lysosomal storage disorder and in need thereof, the method comprising administering to the subject a therapeutically effective amount of the compound or the pharmaceutical composition.

125. A compound of any one of claims 1-104 or a pharmaceutical composition of claim 105 for the manufacture of a medicament for treating a subject with a neurodegenerative disorder and in need thereof, the method comprising administering to the subject a therapeutically effective amount of the compound or the pharmaceutical composition.

126. A compound of any one of claims 1-104 or a pharmaceutical composition of claim 105 for the manufacture of a medicament for treating a subject with an inflammatory disorder and in need thereof, the method comprising administering to the subject a therapeutically effective amount of the compound or the pharmaceutical composition.
